(19) United States
(12) Patent Application Publication
(10) Pub. No.: US 2026/0152733 A1
(43) Pub. Date: Jun. 4, 2026

(54) COMPOSITIONS AND METHODS FOR THE TARGETING OF LPA

(71) Applicant: Scribe Therapeutics Inc., Alameda, CA (US)

(72) Inventors: Dena LEEMAN, Lafayette, CA (US); Sarah DENNY, San Francisco, CA (US); Sean HIGGINS, Alameda, CA (US); Brett T. STAAHL, Tiburon, CA (US); Oleh KRUPA, Oakland, CA (US); Benjamin DEMAREE, Berkeley, CA (US); Matthew HARMS, Alameda, CA (US); Benjamin OAKES, El Cerrito, CA (US); Addison WRIGHT, El Cerrito, CA (US)

(21) Appl. No.: 19/343,806

(22) Filed: Sep. 29, 2025

Related U.S. Application Data
(63) Continuation of application No. PCT/US2024/022042, filed on Mar. 28, 2024.
(60) Provisional application No. 63/492,875, filed on Mar. 29, 2023.
(60) Provisional application No. 63/505,885, filed on Jun. 2, 2023.
(60) Provisional application No. 63/563,109, filed on Mar. 8, 2024.

(51) Int. Cl. C12N 9/22; C12N 15/11; C12N 15/88; C12N 15/90

(52) U.S. Cl. CPC C12N 9/226; C12N 15/11; C12N 15/88; C12N 15/907; C12N 2310/20; C12N 2310/315; C12N 2310/321

(57) ABSTRACT

Provided herein are systems comprising Class 2, Type V CRISPR proteins and guide nucleic acids (gRNA) useful in the modification of a target nucleic acid of a lipoprotein (a) (LPA) gene. The systems are also useful for introduction into cells, for example eukaryotic cells. Also provided are methods for treating subjects having a LPA-related disease and methods for optimizing the encoding sequences.

### CROSS REFERENCE TO RELATED APPLICATIONS

This application is a Continuation of International Application No. PCT/US2024/022042, filed on Mar. 28, 2024, which claims priority to U.S. Provisional Applications No. 63/492,875, filed on Mar. 29, 2023, 63/505,885 filed on Jun. 2, 2023, and 63/563,109 filed on Mar. 8, 2024, the contents of each of which are incorporated herein by reference in their entirety.

### REFERENCE TO AN ELECTRONIC SEQUENCE LISTING

The contents of the electronic sequence listing (SCRB_051_03US_SeqList_ST26.xml; Size: 19,230,224 bytes; and Date of Creation: Sep. 28, 2025) are herein incorporated by reference in its entirety.

### BACKGROUND

Lipoproteins are globular, micelle-like particles that consist of a non-polar core of acylglycerols and cholesteryl esters surrounded by an amphiphilic coating of protein, phospholipid and cholesterol. Lipoproteins have been classified into at least five categories based on their functional and physical properties: chylomicrons, very low-density lipoproteins (VLDL), intermediate density lipoproteins (IDL), low density lipoproteins (LDL), and high-density lipoproteins (HDL). Chylomicrons transport dietary lipids from intestine to tissues, VLDLs, IDLs and LDLs all transport triacylglycerols and cholesterol from the liver to tissues, and HDLs transport endogenous cholesterol from tissues to the liver.

Lipoprotein particles undergo continuous metabolic processing and have variable properties and compositions. Lipoprotein densities increase without increasing particle diameter because the density of their outer coatings is less than that of the inner core. The protein components of lipoproteins are known as apolipoproteins and at least nine different forms of apolipoproteins are distributed in significant amounts among the various human lipoproteins.

The lipoprotein (a) particle (Lp (a)) was identified nearly 50 years ago and is comprised of a highly unique LDL particle in which one apolipoprotein B (apoB) protein is linked via a disulfide bond to a single apolipoprotein (a) (apo (a)) protein. The apo (a) protein shares a high degree of homology with plasminogen, particularly within the kringle IV (KIV) type 2 repetitive domain. Generally, levels of circulating Lp (a) are inversely proportional to the number of kringle IV type 2 variable repeats present in the molecule and, as both alleles are co-expressed within individuals, can display heterozygous isoform profiles in the plasma (Kraft et al., Eur J Hum. Genet. 4 (2): 74 (1996)). It is thought that this kringle repeat domain in apo (a) may be responsible for its pro-thrombotic and anti-fibrinolytic properties, potentially enhancing atherosclerotic progression. Importantly, recent genetic association studies revealed that Lp (a) was an independent risk factor for myocardial infarction, stroke, peripheral vascular disease and abdominal aortic aneurysm (Rifai et al., Clin. Chem. 2004; 50:1364-71; Erqou et al., JAMA 2009; 302:412-23; Kamstrup et al., Circulation 2008; 117: 176-84). Further, in the recent Precocious Coronary Artery Disease (PROCARDIS) study, Clarke et al. (Clarke et al., NEJM (2009) 361:2518-2528) described robust and independent associations between coronary heart disease and plasma Lp (a) concentrations. To date, therapeutic strategies to treat cardiovascular disease by directly targeting Lp (a) and/or apo (a) levels have been limited. Thus, there remains a need for improved compositions and methods to regulate Lp (a).

### SUMMARY

Aspects of the present disclosure are directed to systems and methods of modulating expression of a lipoprotein (a) (IPA) gene target nucleic acid in a cell.

The present disclosure provides systems comprising or encoding engineered CasX proteins and guide ribonucleic acid variants (gRNA) that have utility in the modification of a target nucleic acid of a lipoprotein (a) (LPA) gene in eukaryotic cells. The systems and compositions are useful in a variety of methods for target nucleic acid modification, which methods are also provided. The present disclosure also provides vectors and LNP formulations for the delivery of the systems to cells for the modification of the LPA gene. The present disclosure also provides methods for treating subjects having an LPA-related disease. The present disclosure also provides methods for optimizing the encoding sequences.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The contents of WO 2020247882, WO 2020247883, WO 2021113772, WO 2022120095, WO 2022125843, WO 2022261150, WO 2022261149, WO 2023049872, WO 2023235818, WO 2023240027, and WO 2023240162, which disclose CasX variants and gRNA variants, and methods of delivering same, are hereby incorporated by reference in their entirety.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of the relative locations in the human LPA locus that the 142 TTC spacers target, as described in Example 1. Spacers are indicated by black vertical bars. “KIV” denotes a Kringle IV domain, followed by the domain type (e.g., “KIV-1”=Kringle IV Type 1). “KV” denotes a Kringle V domain.

FIG. 2 is a bar graph showing the quantification of percent editing measured as indel rate detected by next-generation sequencing (NGS) at the mouse PCSK9 locus in Hepal-6 cells transfected with the indicated engineered CasX mRNAs and targeting spacers and harvested at 20 hours post-transfection, as described in Example 5.

FIG. 3A is a plot illustrating the percent reduction of secreted PCSK9 level, relative to the non-targeting (NT) control, for primary human hepatocytes from lot #31 treated with the indicated doses of lipid nanoparticles (LNPs) formulated with CasX 515 or CasX 812 mRNA and a PCSK9-targeting gRNA with spacer 6.1, as described in Example 6.

FIG. 3B is a plot illustrating the percent reduction of secreted PCSK9 level, relative to the non-targeting (NT) control, for primary human hepatocytes from lot #31 treated with the indicated doses of LNPs formulated with CasX 515 or CasX 812 mRNA and a PCSK9-targeting gRNA with spacer 6.8, as described in Example 6.

FIG. 3C is a plot illustrating the percent reduction of secreted PCSK9 level, relative to the non-targeting (NT) control, for primary human hepatocytes from lot #51 treated with the indicated doses of LNPs formulated with CasX 515 or CasX 812 mRNA and a PCSK9-targeting gRNA with spacer 6.1, as described in Example 6.

FIG. 3D is a plot illustrating the percent reduction of secreted PCSK9 level, relative to the non-targeting (NT) control, for primary human hepatocytes from lot #51 treated with the indicated doses of LNPs formulated with CasX 515 or CasX 812 mRNA and a PCSK9-targeting gRNA with spacer 6.8, as described in Example 6.

FIG. 4A is a plot depicting the results of an editing assay measured as indel rate detected by NGS at the PCSK9 locus in primary human hepatocytes from lot #31 treated with the indicated doses of LNPs formulated with CasX 515 or CasX 812 mRNA and a PCSK9-targeting gRNA with spacer 6.8, as described in Example 6.

FIG. 4B is a plot depicting the results of an editing assay measured as indel rate detected by NGS at the PCSK9 locus in primary human hepatocytes from lot #51 treated with the indicated doses of LNPs formulated with CasX 515 or CasX 812 mRNA and a PCSK9-targeting gRNA with spacer 6.1, as described in Example 6.

FIG. 4C is a plot depicting the results of an editing assay measured as indel rate detected by NGS at the PCSK9 locus in primary human hepatocytes from lot #51 treated with the indicated doses of LNPs formulated with CasX 515 or CasX 812 mRNA and a PCSK9-targeting gRNA with spacer 6.8, as described in Example 6.

FIG. 5A is a schematic illustrating versions 1-3 (“V1-V3”) of chemical modifications made to gRNA scaffold variant 235, as described in Example 5. Structural motifs are highlighted. Standard ribonucleotides are depicted as open circles, and 2′OMe-modified ribonucleotides are depicted as black circles. Phosphorothioate bonds are indicated with * below or beside the bond. For the v2 profile, the addition of three 3′ uracils (3 UUU) is annotated with “U”s in the relevant circles.

FIG. 5B is a schematic illustrating versions 4-6 (“V4-V6”) of chemical modifications made to gRNA scaffold variant 235, as described in Example 5. Structural motifs are highlighted. Standard ribonucleotides are depicted as open circles, and 2′OMe-modified ribonucleotides are depicted as black circles. Phosphorothioate bonds are indicated with * below or beside the bond.

FIG. 6 is a plot illustrating the quantification of percent knockout of B2M in HepG2 cells co-transfected with 100 ng of CasX 491 mRNA and with the indicated doses of end-modified (v1) or unmodified (v0) B2M-targeting gRNAs with spacer 7.37, as described in Example 5. Editing level was determined by flow cytometry as the population of cells with loss of surface presentation of the HLA complex due to successful editing at the B2M locus.

FIG. 7 is a schematic illustrating versions 7-9 (“V7-V9”) of chemical modifications made to gRNA scaffold variant 316, as described in Example 7. Structural motifs are highlighted. Standard ribonucleotides are depicted as open circles, and 2′OMe-modified ribonucleotides are depicted as black circles. Phosphorothioate bonds are indicated with * below or beside the bond.

FIG. 8A is a schematic of gRNA scaffold variant 174, as described in Example 7. Structural motifs are highlighted (gRNA scaffold variant 174 sequence: SEQ ID NO: 464).

FIG. 8B is a schematic of gRNA scaffold variant 235, as described in Example 7. Highlighted structural motifs are the same as in FIG. 8A. The differences between variant 174 and variant 235 lie in the extended stem motif and several single-nucleotide changes (indicated with asterisks). Variant 316 maintains the shorter extended stem from variant 174 but harbors the four substitutions found in scaffold 235 (gRNA scaffold variant 235 sequence: SEQ ID NO: 465).

FIG. 8C is a schematic of gRNA scaffold variant 316, as described in Example 7. Highlighted structural motifs are the same as in FIG. 8A (gRNA scaffold variant 316 sequence: SEQ ID NO: 466). Variant 316 maintains the shorter extended stem from variant 174 (FIG. 8A) but harbors the four substitutions found in scaffold 235 (FIG. 8B).

FIG. 9 is a plot displaying a correlation between indel rate (depicted as edit fraction) at the PCSK9 locus as measured by NGS (x-axis) and secreted PCSK9 levels (ng/ml) detected by enzyme-linked immunosorbent assay (ELISA) (y-axis) in HepG2 cells lipofected with CasX 491 mRNA and PCSK9-targeting gRNAs containing the indicated scaffold variant and spacer combination, as described in Example 7.

FIG. 10A is a plot depicting the results of an editing assay measured as indel rate detected by NGS at the human B2M locus in HepG2 cells treated with the indicated doses of LNPs formulated with CasX 491 mRNA and the indicated B2M-targeting gRNA, as described in Example 7.

FIG. 10B is a plot illustrating the quantification of percent knockout of B2M in HepG2 cells treated with the indicated doses of LNPs formulated with CasX 491 mRNA and the indicated B2M-targeting gRNA, as described in Example 7. Editing level was determined by flow cytometry as population of cells that did not have surface presentation of the HLA complex due to successful editing at the B2M locus.

FIG. 11A is a plot depicting the results of an editing assay measured as indel rate detected by NGS at the mouse ROSA26 locus in Hepal-6 cells treated with the indicated doses of LNPs formulated with CasX 676 mRNA #2 and the indicated ROSA26-targeting gRNA with either the v1 or v5 modification profile, as described in Example 7.

FIG. 11B is a plot illustrating the quantification of percent editing measured as indel rate detected by NGS at the ROSA26 locus in mice treated with LNPs formulated with CasX 676 mRNA #2 and the indicated chemically-modified ROSA26-targeting gRNA, as described in Example 7.

FIG. 12 is a bar graph showing the results of the editing assay measured as indel rate detected by NGS as the mouse PCSK9 locus in mice treated with LNPs formulated with CasX 676 mRNA #1 and the indicated chemically-modified PCSK9-targeting gRNA, as described in Example 7. Untreated mice served as experimental control.

FIG. 13A is a schematic illustrating versions 1-3 (“V1-V3”) of chemical modifications made to gRNA scaffold variant 316, as described in Example 7. Structural motifs are highlighted. Standard ribonucleotides are depicted as open circles, and 2′OMe-modified ribonucleotides are depicted as black circles. Phosphorothioate bonds are indicated with * below or beside the bond.

FIG. 13B is a schematic illustrating versions 4-6 (“V4-V6”) of chemical modifications made to gRNA scaffold variant 316, as described in Example 7. Structural motifs are highlighted. Standard ribonucleotides are depicted as open circles, and 2′OMe-modified ribonucleotides are depicted as black circles. Phosphorothioate bonds are indicated with * below or beside the bond.

FIG. 14 is a bar graph showing the level of apo (a) secretion in primary human hepatocytes treated with mRNA encoding CasX 515 and indicated gRNA, relative to cells treated with a non-targeting (NT) gRNA. Treatment with LPA targeted gRNA shows reduction greater than 90% as measured by protein analysis normalized to total secreted protein, as described in Example 9.

FIG. 15 is a plot illustrating the quantification of editing rate as the percent of on-target editing of LPA in HEK293 cells nucleofected with one of 7 gRNAs targeting LPA and a plasmid encoding CasX 515 as described in Example 10. Guide RNA IDs are displayed in order of decreasing estimated guide specificity.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the disclosures claimed herein. It should be understood that various alternatives to the embodiments described herein may be employed in practicing the embodiments of the disclosure. It is intended that the claims define the scope of the disclosure and that methods and structures within the scope of these claims and their equivalents be covered thereby.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

As used in this specification and the appended claims, the singular forms “a,” “an,” and “the” include plural referents unless the context clearly dictates otherwise. Thus, for example, reference to “a host cell” includes two or more such host cells, reference to “an engineered CasX protein” includes one or more engineered CasX protein(s), reference to “a nucleic acid sequence” includes one or more nucleic acid sequences, and the like.

As used herein, the term “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also include any and all possible subranges and combinations of subranges thereof. Furthermore, as will be understood by one skilled in the art, a range includes each individual member. Thus, for example, a group having 1-3 members refers to groups having 1, 2, or 3 members. Similarly, a group having 1-5 members refers to groups having 1, 2, 3, 4, or 5 members, and so forth.

The term “combinations thereof” includes every possible combination of elements to which the term refers.

The term “exemplary” as used herein, refers to an example or illustration, and is not intended to imply any preference or value.

The term “CasX protein,” as used herein, refers to a family of proteins, including e.g., all naturally-occurring CasX proteins (“reference CasX”), as well as engineered CasX proteins with sequence modifications possessing one or more improved characteristics relative to a CasX protein from which it was derived, described more fully, herein below.

The term “specificity” as used herein with respect to editing refers to the ratio of average level of on-targeting editing divided by the average level of off-target editing.

The term “off-target effects” as used herein refers to unintended cleavage and mutations at untargeted genomic sites showing a similar but not an identical sequence compared to the target site. In some embodiments the off-target effects are determined in an in vitro cell-free assay. In some embodiments the off-target effects are determined in a cell-based assay.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary.” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid to be specifically hybridizable; it can have at least about 70%, at least about 80%, or at least about 90%, or at least about 95% sequence identity and still hybridize to the target nucleic acid. Moreover, a polynucleotide may hybridize over one or more segments such that intervening or adjacent segments are not involved in the hybridization event (e.g., a loop structure or hairpin structure, a ‘bulge’, ‘bubble’ and the like). Thus, the skilled artisan will understand that while individual bases within a sequence may not be complementary to another sequence, the sequence as a whole is still considered to be complementary.

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation: the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “accessory element” is used interchangeably herein with the term “accessory sequence,” and is intended to include coding and non-coding sequences that enhance expression, trafficking of the nucleic acid, or the function of mRNA or protein and include, inter alia, polyadenylation signals (poly (A) signal), enhancer elements, introns, posttranscriptional regulatory elements (PTREs), nuclear localization signals (NLS), deaminases, DNA glycosylase inhibitors, additional promoters, factors that stimulate CRISPR-mediated homology-directed repair (e.g. in cis or in trans), self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PTRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, “Lp (a)” or “lipoprotein (a)” comprises apo (a) and an LDL-like particle containing apoB. The apo (a) protein is linked to the apoB by a disulfide bond.

As used herein, “apolipoprotein A” or “apo (a)” means a protein sequence comprising apo (a). The apo (a) is encoded by the LPA gene.

As used herein, “apolipoprotein B” or “apoB” means a protein sequence comprising apoB. The apoB protein is encoded by the APOB gene.

As used herein, “lipoprotein”, such as VLDL, LDL and HDL, refers to a group of proteins found in the serum, plasma and lymph and are important for lipid transport. The chemical composition of each lipoprotein differs, for example, in that the HDL has a higher proportion of protein versus lipid, whereas the VLDL has a lower proportion of protein versus lipid.

As used herein, “atherosclerosis” means a hardening of the arteries affecting large and medium-sized arteries and is characterized by the presence of fatty deposits. The fatty deposits are called “atheromas” or “plaques,” which consist mainly of cholesterol and other fats, calcium and scar tissue, and damage the lining of arteries.

As used herein, “coronary heart disease (CHD)” means a narrowing of the small blood vessels that supply blood and oxygen to the heart, which is often a result of atherosclerosis.

As used herein, “dyslipidemia” refers to a disorder of lipid and/or lipoprotein metabolism, including lipid and/or lipoprotein overproduction or deficiency. Dyslipidemias can be manifested by elevation of lipids such as chylomicron, cholesterol and triglycerides as well as lipoproteins such as low-density lipoprotein (LDL) cholesterol.

As used herein, “high density lipoprotein-C” or “HDL-C” means cholesterol associated with high-density lipoprotein particles. Concentration of HDL-C in serum (or plasma) is typically quantified in mg/dL or nmol/L. “Serum HDL-C” and “plasma HDL-C” mean HDL-C in serum and plasma, respectively.

As used herein, “low density lipoprotein-cholesterol (LDL-C)” means cholesterol carried in low density lipoprotein particles. Concentration of LDL-C in serum (or plasma) is typically quantified in mg/dL or nmol/L. “Serum LDL-C” and “plasma LDL-C” mean LDL-C in the serum and plasma, respectively.

As used herein, “hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. 148:36 (1988)).

As used herein, “hyperlipidemia” or “hyperlipemia” is a condition characterized by elevated serum lipids or circulating (plasma) lipids. This condition manifests an abnormally high concentration of fats. The lipid fractions in the circulating blood are cholesterol, low-density lipoproteins, very low density lipoproteins, chylomicrons and triglycerides. The Fredrickson classification of hyperlipidemias is based on the pattern of TG and cholesterol-rich lipoprotein particles, as measured by electrophoresis or ultracentrifugation and is commonly used to characterize primary causes of hyperlipidemias such as hypertriglyceridemia.

As used herein, “triglyceride” or “TG” means a lipid or neutral fat consisting of glycerol combined with three fatty acid molecules.

As used herein, “hypertriglyceridemia” means a condition characterized by elevated triglyceride levels. Its etiology includes primary (i.e., genetic causes) and secondary (other underlying causes such as diabetes, metabolic syndrome/insulin resistance, obesity, physical inactivity, cigarette smoking, excess alcohol, and a diet very high in carbohydrates) factors or, most often, a combination of both

As used herein, “diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

As used herein, “diabetic dyslipidemia” or “type 2 diabetes with dyslipidemia” means a condition characterized by Type 2 diabetes, reduced HDL-C, elevated triglycerides (TG), and elevated small, dense LDL particles.

As used herein, “lipid nanoparticle” or “LNP” refers to a particle having at least one dimension on the order of nanometers (e.g., 1-1,000 nm) comprising one or more lipids (e.g., cationic lipids, non-cationic lipids, helper phospholipids, and PEG-modified lipids), as well as cholesterol. Specific components of LNP are described more fully, below. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells; e.g., an adverse immune response.

As used herein, “lipid nanoparticle” refers to particles having at least one dimension on the order of nanometers (e.g., 1-1,000 nm) comprising one or more lipids (e.g., cationic lipids, non-cationic lipids, and PEG-modified lipids). In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

The disclosure provides systems and methods useful for editing a target nucleic acid sequence. As used herein “editing” is used interchangeably with “modifying” and “modification” and includes but is not limited to cleaving, nicking, deleting, knocking in, knocking out, and the like.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, leading to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) or insertion of nucleotide sequence near the site of the double-strand break.

As used herein “micro-homology mediated end joining” (MMEJ) refers to a mutagenic double-strand break repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “protein,” “polypeptide,” and “peptide” are used interchangeably herein to refer to a polymer of amino acid residues linked via peptide bonds and which may be composed of two or more polypeptide chains. Polypeptide chains can be of any length, and in some embodiments can include coded and non-coded amino acids, chemically or biochemically modified or derivatized amino acids, and polypeptides having modified peptide backbones. The term includes fusion proteins, including, but not limited to, fusion proteins with a heterologous amino acid sequences. Typically, the terms “polypeptide,” “protein,” and/or “peptide” refer to a polymer of at least two amino acid monomers joined together through peptide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. In some cases, a protein can be a portion of a larger protein, for example, a domain, a subdomain, or a motif of the protein. In some cases, a protein can be a variant (or mutation) of a protein, wherein one or more amino acid residues are inserted into, deleted from, and/or substituted into the naturally occurring, or reference, amino acid sequence of a protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

A protein or a variant thereof can be naturally occurring or recombinant. Methods for detection and/or measurement of polypeptides in biological material are well known in the art and include, but are not limited to, Western-blotting, flow cytometry, ELISAs, RIAS, and various proteomics techniques. An exemplary method to measure or detect a polypeptide is an immunoassay, such as e.g., an ELISA This type of protein quantitation can be based on an antibody capable of capturing a specific antigen, and a second antibody capable of detecting the captured antigen. Exemplary assays for detection and/or measurement of polypeptides/proteins are described e.g., in Harlow, E. and Lane, D. Antibodies: A Laboratory Manual, (1988), Cold Spring Harbor Laboratory Press.

In the context of proteins, the term “fragment,” or equivalent terms refers, as used herein refers to a portion of a protein that has less than the full length of the protein and optionally maintains the function of the protein.

A “vector” or “expression vector” is a replicon, such as plasmid, phage, virus, or cosmid, which can include another DNA segment, i.e., an expression cassette, so as to bring about the replication or expression of the other DNA segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., a vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., a vector.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The terms “therapeutically effective amount,” “effective amount,” and “therapeutically effective dose,” are used interchangeably herein, to refer to an amount of a drug or a biologic, alone or as a part of a composition, that is capable of having any detectable, beneficial effect on any symptom, aspect, measured parameter or characteristics of a disease state or condition when administered in one or repeated doses to a subject in need thereof, such e.g., as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

As used herein, “treatment” or “treating,” are used interchangeably herein and refer to an approach for obtaining beneficial or desired results, including but not limited to a therapeutic benefit and/or a prophylactic benefit. Therapeutic benefit refers to eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

A “subject” is a mammal. Mammals include, but are not limited to, domesticated animals, non-human primates, humans, dogs, rabbits, mice, rats and other rodents.

The term “low-density lipoprotein (LDL)” refers to one of the five major groups of lipoprotein, from least dense (lower weight-volume ratio particles) to most dense (larger weight-volume ratio particles): chylomicrons, very low-density lipoproteins (VLDL), low-density lipoproteins (LDL), intermediate-density lipoproteins (IDL), and high-density lipoproteins (HDL). Lipoproteins transfer lipids (fats) around the body in the extracellular fluid thereby facilitating the transfer of fats to the cells body via receptor-mediated endocytosis. An LDL particle is about 220-275 angstroms in diameter.

“Low-density lipoprotein (LDL) receptor” refers to a receptor protein of 839 amino acids (after removal of 21-amino acid signal peptide) that mediates the endocytosis of cholesterol-rich LDL particles. It is a cell-surface receptor that recognizes the apoprotein B100 and apoE protein found in chylomicron remnants and VLDL remnants (IDL) resulting in the binding and endocytosis of LDL-cholesterol. This process occurs in all nucleated cells, but mainly in the liver which removes approximately 70% of LDL from the circulation. The human LDLR gene is described in part in the NCBI database (ncbi.nlm.nih.gov) as Reference Sequence NG_009060.1, which is incorporated by reference herein.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4^{th} Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Systems for Genetic Editing of LPA Genes

In a first aspect, the present disclosure provides systems comprising an engineered CasX nuclease protein and one or more guide ribonucleic acids (gRNA), as well as nucleic acids encoding the engineered CasX nuclease proteins and gRNA, for use in modifying an LPA gene, inclusive of coding and non-coding regions. As used herein, a “system”, used interchangeably with “composition”, can comprise an engineered CasX nuclease protein and one or more gRNAs of the disclosure as gene editing pairs, nucleic acids (DNA, RNA or vectors) encoding the engineered CasX nuclease proteins and gRNA, as well as vectors or lipid nanoparticles (LNP) comprising the nucleic acids or engineered CasX nuclease protein and one or more gRNAs the disclosure.

The LPA gene encodes a serine proteinase that inhibits the activity of tissue-type plasminogen activator I. The encoded protein constitutes a substantial portion of lipoprotein (a). Apo (a) is known to be proteolytically cleaved, leading to the formation of the so-called mini-Lp (a). Apo (a) fragments accumulate and attach to atherosclerotic lesions and promote thrombogenesis.

The gene comprises the sequence that spans chr6: 160,531,482-160,664 of the human genome (GRCh38/hg38) (the notation refers to the chromosome 6 (chr6), starting at the 160,531,482 bp to 160,664,275 bp on chromosome 6. The human LPA gene is described in part in the NCBI database (ncbi.nlm.nih.gov) as Reference Sequence NG_016147.1, which is incorporated by reference herein. The LPA locus has 39 exons that produces an mRNA of 13938 bp, encoding apo (a), having 2040 amino acids, the main constituent of lipoprotein (a) (Lp (a)).

The human LPA gene (HGNC: 6667 NCBI Entrez Gene: 4018) encodes a protein (P08519) having the sequence

(SEQ ID NO: 543)
MEHKEVVLLLLLFLKSAAPEQSHVVQDCYHGDGQSYRGTYSTTVTGRTCQAWSSMTPHQHNRTTENYPN
AGLIMNYCRNPDAVAAPYCYTRDPGVRWEYCNLTQCSDAEGTAVAPPTVTPVPSLEAPSEQAPTEQRPG
VQECYHGNGQSYRGTYSTTVTGRTCQAWSSMTPHSHSRTPEYYPNAGLIMNYCRNPDAVAAPYCYTRDP
GVRWEYCNLTQCSDAEGTAVAPPTVTPVPSLEAPSEQAPTEQRPGVQECYHGNGQSYRGTYSTTVTGRT
CQAWSSMTPHSHSRTPEYYPNAGLIMNYCRNPDAVAAPYCYTRDPGVRWEYCNLTQCSDAEGTAVAPPT
VTPVPSLEAPSEQAPTEQRPGVQECYHGNGQSYRGTYSTTVTGRTCQAWSSMTPHSHSRTPEYYPNAGL
IMNYCRNPDAVAAPYCYTRDPGVRWEYCNLTQCSDAEGTAVAPPTVTPVPSLEAPSEQAPTEQRPGVQE
CYHGNGQSYRGTYSTTVTGRTCQAWSSMTPHSHSRTPEYYPNAGLIMNYCRNPDAVAAPYCYTRDPGVR
WEYCNLTQCSDAEGTAVAPPTVTPVPSLEAPSEQAPTEQRPGVQECYHGNGQSYRGTYSTTVTGRTCQA
WSSMTPHSHSRTPEYYPNAGLIMNYCRNPDAVAAPYCYTRDPGVRWEYCNLTQCSDAEGTAVAPPTVTP
VPSLEAPSEQAPTEQRPGVQECYHGNGQSYRGTYSTTVTGRTCQAWSSMTPHSHSRTPEYYPNAGLIMN
YCRNPDAVAAPYCYTRDPGVRWEYCNLTQCSDAEGTAVAPPTVTPVPSLEAPSEQAPTEQRPGVQECYH
GNGQSYRGTYSTTVTGRTCQAWSSMTPHSHSRTPEYYPNAGLIMNYCRNPDPVAAPYCYTRDPSVRWEY
CNLTQCSDAEGTAVAPPTITPIPSLEAPSEQAPTEQRPGVQECYHGNGQSYQGTYFITVTGRTCQAWSS
MTPHSHSRTPAYYPNAGLIKNYCRNPDPVAAPWCYTTDPSVRWEYCNLTRCSDAEWTAFVPPNVILAPS
LEAFFEQALTEETPGVQDCYYHYGQSYRGTYSTTVTGRTCQAWSSMTPHQHSRTPENYPNAGLTRNYCR
NPDAEIRPWCYTMDPSVRWEYCNLTQCLVTESSVLATLTVVPDPSTEASSEEAPTEQSPGVQDCYHGDG
QSYRGSFSTTVTGRTCQSWSSMTPHWHQRTTEYYPNGGLTRNYCRNPDAEISPWCYTMDPNVRWEYCNL
TQCPVTESSVLATSTAVSEQAPTEQSPTVQDCYHGDGQSYRGSFSTTVTGRTCQSWSSMTPHWHQRTTE
YYPNGGLTRNYCRNPDAEIRPWCYTMDPSVRWEYCNLTQCPVMESTLLTTPTVVPVPSTELPSEEAPTE
NSTGVQDCYRGDGQSYRGTLSTTITGRTCQSWSSMTPHWHRRIPLYYPNAGLTRNYCRNPDAEIRPWCY
TMDPSVRWEYCNLTRCPVTESSVLTTPTVAPVPSTEAPSEQAPPEKSPVVQDCYHGDGRSYRGISSTTV
TGRTCQSWSSMIPHWHQRTPENYPNAGLTENYCRNPDSGKQPWCYTTDPCVRWEYCNLTQCSETESGVL
ETPTVVPVPSMEAHSEAAPTEQTPVVRQCYHGNGQSYRGTFSTTVTGRTCQSWSSMTPHRHQRTPENYP
NDGLTMNYCRNPDADTGPWCFTMDPSIRWEYCNLTRCSDTEGTVVAPPTVIQVPSLGPPSEQDCMFGNG
KGYRGKKATTVTGTPCQEWAAQEPHRHSTFIPGTNKWAGLEKNYCRNPDGDINGPWCYTMNPRKLFDYC
DIPLCASSSFDCGKPQVEPKKCPGSIVGGCVAHPHSWPWQVSLRTRFGKHFCGGTLISPEWVLTAAHCL
KKSSRPSSYKVILGAHQEVNLESHVQEIEVSRLFLEPTQADIALLKLSRPAVITDKVMPACLPSPDYMV
TARTECYITGWGETQGTFGTGLLKEAQLLVIENEVCNHYKYICAEHLARGTDSCQGDSGGPLVCFEKDK
YILQGVTSWGLGCARPNKPGVYARVSRFVTWIEGMMRNN.

In some embodiments, the disclosure provides systems specifically designed to modify the LPA gene in eukaryotic cells. In some cases, the systems of the disclosure are designed to knock-down or knock-out the LPA gene. Generally, any portion of the LPA gene can be targeted using the programable compositions and methods provided herein, described more fully, herein.

In some embodiments, the disclosure provides systems comprising one or more engineered CasX proteins or nucleic acids encoding the engineered CasX proteins, and one or more guide nucleic acid variants (gRNA) as a CasX:gRNA system designed to target and edit specific locations in the target nucleic acid sequence of the LPA gene. In some embodiments, the disclosure provides vectors encoding the CasX:gRNA components, which, when delivered to the target cells, are expressed and associate as gene editing pairs capable of modifying the LPA gene. In some embodiments, the disclosure provides LNP that comprise an mRNA that encodes the CasX component and one or more gRNA that, when delivered to the target cells, express the CasX, which associates with the gRNA as a gene editing pair capable of modifying the LPA gene. Each of these components and their use in the editing of the LPA gene is described herein, below.

### III. CasX Proteins for Modifying a Target Nucleic Acid of an LPA Gene

The present disclosure provides engineered CasX nuclease proteins, and nucleic acids encoding same, that can be used to modify a target nucleic acid of an LPA gene in eukaryotic cells.

The engineered CasX proteins employed in the systems described herein are Class 2, Type V nucleases. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize a TC motif protospacer adjacent motif (PAM) 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the CasX proteins of the embodiments recognize a 5′-TC PAM motif and produce staggered ends cleaved solely by the RuvC domain.

Engineered CasX proteins of the disclosure comprise the following domains: a non-target strand binding (NTSB) domain, a target strand loading (TSL) domain, a helical I domain, a helical II domain, an oligonucleotide binding domain (OBD), and a RuvC DNA cleavage domain, and, in some cases, domains can be further divided into subdomains, as listed in Tables 2 and 3.

In some embodiments, an engineered CasX protein can bind and/or modify (e.g., catalyze a single strand break (a “nickase”), or catalyze a double strand break) a target nucleic acid at a specific sequence targeted by an associated gRNA, which hybridizes to a sequence within the target nucleic acid sequence. In some embodiments, the engineered CasX comprises a nuclease domain having double-stranded cleavage activity that generates a double-stranded break within 18-26 nucleotides 5′ of a PAM site on the target strand and 10-18 nucleotides 3′ on the non-target strand, resulting in overhangs that can facilitate a higher degree of editing efficiency or insertion of a donor template nucleic acid by HDR or HITI repair mechanisms of the host cell, compared to other CRISPR systems.

a. Reference CasX Proteins

The disclosure provides naturally-occurring CasX proteins (referred to herein as a “reference CasX protein”), which were subsequently modified to create the engineered CasX proteins of the disclosure. For example, reference CasX proteins can be isolated from naturally occurring prokaryotes, such as Deltaproteobacteria, Planctomycetes, or Candidatus Sungbacteria species. A reference CasX protein (interchangeably referred to herein as a reference CasX polypeptide) is a Class 2, Type V CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter, and comprises a sequence of SEQ ID NO: 1.

In some cases, a reference CasX protein is isolated or derived from Planctomycetes, and comprises a sequence of SEQ ID NO: 2.

In some cases, a reference CasX protein is isolated or derived from Candidatus Sungbacteria, and comprises a sequence of SEQ ID NO: 3.

b. Engineered CasX Proteins

The present disclosure provides highly-modified engineered CasX proteins having multiple mutations relative to one or more reference CasX proteins. Any changes in the amino acid sequence of a reference CasX protein which results in a CasX and that leads to an improved characteristic relative to the reference CasX protein is considered an engineered CasX protein of the disclosure, provided the CasX retains the ability to form an RNP with a gRNA and retains nuclease activity. Exemplary improved characteristics of the engineered CasX embodiments, relative to reference CasX comprising sequences of SEQ ID NOS: 1-3 (described, supra) include, but are not limited to improved ability to utilize a greater spectrum of PAM sequences in the editing and/or binding of target nucleic acid, increased nuclease activity, improved editing efficiency, improved editing specificity for the target nucleic acid, decreased off-target editing or cleavage, increased percentage of a eukaryotic genome that can be efficiently edited, increased activity of the nuclease, improved ability to form cleavage-competent ribonucleoprotein (RNP) complexes with gRNA, and improved ribonucleoprotein (RNP) complex stability. In particular, the engineered CasX proteins of the disclosure have an enhanced ability to efficiently edit and/or bind target DNA, when complexed with a guide RNA scaffold as an RNP, utilizing a PAM TC motif, including PAM sequences selected from TTC, ATC, GTC, or CTC, compared to an RNP of a reference CasX protein and a reference gRNA. In the foregoing, the PAM sequence is located at least l nucleotide 5′ to the non-target strand of the protospacer having identity with the targeting sequence of the gRNA in an assay system compared to the editing efficiency and/or binding of an RNP comprising the reference CasX protein and reference gRNA in a comparable assay system. In the foregoing embodiments, the one or more of the improved characteristics of the engineered CasX is at least about 1.1 to about 100,000-fold improved relative to the reference CasX protein of SEQ ID NO: 1, SEQ ID NO: 2, or SEQ ID NO: 3, when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3.

The engineered CasX protein can comprise one or more amino acid substitutions, insertions, deletions, swapped domains from a second CasX protein, or any combinations thereof, relative to a reference CasX protein sequence or a parental CasX variant protein from which it was derived. In some embodiment, the disclosure provides engineered CasX proteins wherein the engineered CasX protein comprises a RuvC cleavage domain, wherein the RuvC cleavage domain comprises the sequence of amino acids 648-812 of SEQ ID NO: 2 with one or more amino acid modifications relative to the RuvC cleavage domain sequence. In some embodiments, the one or more amino acid modifications of the RuvC domain comprise a modification at a position selected from the group consisting of 1658, A708, and P793 relative to SEQ ID NO: 2. Mutations can be introduced in any one or more domains of the reference CasX protein or in a CasX variant to result in an engineered CasX protein, and may include, for example, deletion of part or all of one or more domains, or one or more amino acid substitutions, deletions, or insertions in any domain of the reference CasX protein or the CasX variant protein from which it was derived.

In some embodiments, an engineered CasX protein comprises a sequence of SEQ ID NOS: 4-7, as set forth in Table 1. In some embodiments, an engineered CasX protein consists of a sequence of SEQ ID NOS: 4-7, as set forth in Table 1. In other embodiments, an engineered CasX protein comprises a sequence at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 4, as set forth in Table 1. In some embodiments, an engineered CasX protein comprises a sequence of SEQ ID NO: 4. In some embodiments, an engineered CasX protein comprises a sequence of SEQ ID NO: 5, or a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto. In some embodiments, an engineered CasX protein comprises a sequence of SEQ ID NO: 5. In some embodiments, an engineered CasX protein comprises a sequence of SEQ ID NO: 6, or a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto. In some embodiments, an engineered CasX protein comprises a sequence of SEQ ID NO: 6. In some embodiments, an engineered CasX protein comprises a sequence of SEQ ID NO: 7, or a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto. In some embodiments, an engineered CasX protein comprises a sequence of SEQ ID NO: 7. In some embodiments, the foregoing engineered CasX proteins retain the functional properties of the ability to form an RNP with a gRNA and retain nuclease activity.

TABLE 1
Engineered CasX Protein Sequences

| SEQ | Variant | |
|---|---|---|
| ID NO | No. | Amino Acid Sequence |
| 4 | 491 | QEIKRINKIRRRLVKDSNTKKAGKTGPMKTLLVRVMTPDLRERLENLRKKPENIPQPI |
| | | SNTSRANLNKLLTDYTEMKKAILHVYYEEFQKDPVGLMSRVAQPASKKIDQNKLKPEM |
| | | DEKGNLTTAGFACSQCGQPLFVYKLEQVSEKGKAYTNYFGRCNVAEHEKLILLAQLKP |
| | | EKDSDEAVTYSLGKFGQRALDFYSIHVTKESTHPVKPLAQIAGNRYASGPVGKALSDA |
| | | CMGTIASFLSKYQDIIIEHQKVVKGNQKRLESLRELAGKENLEYPSVTLPPQPHTKEG |
| | | VDAYNEVIARVRMWVNLNLWQKLKLSRDDAKPLLRLKGFPSFPLVERQANEVDWWDMV |
| | | CNVKKLINEKKEDGKVFWQNLAGYKRQEALRPYLSSEEDRKKGKKFARYQLGDLLLHL |
| | | EKKHGEDWGKVYDEAWERIDKKVEGLSKHIKLEEERRSEDAQSKAALTDWLRAKASFV |
| | | IEGLKEADKDEFCRCELKLQKWYGDLRGKPFAIEAENSILDISGFSKQYNCAFIWQKD |
| | | GVKKLNLYLIINYFKGGKLRFKKIKPEAFEANRFYTVINKKSGEIVPMEVNFNFDDPN |
| | | LIILPLAFGKRQGREFIWNDLLSLETGSLKLANGRVIEKTLYNRRTRQDEPALFVALT |
| | | FERREVLDSSNIKPMNLIGVDRGENIPAVIALTDPEGCPLSRFKDSLGNPTHILRIGE |
| | | SYKEKQRTIQAKKEVEQRRAGGYSRKYASKAKNLADDMVRNTARDLLYYAVTQDAMLI |
| | | FENLSRGFGRQGKRTFMAERQYTRMEDWLTAKLAYEGLSKTYLSKTLAQYTSKTCSNC |
| | | GFTITSADYDRVLEKLKKTATGWMTTINGKELKVEGQITYYNRYKRQNVVKDLSVELD |
| | | RLSEESVNNDISSWTKGRSGEALSLLKKRFSHRPVQEKFVCLNCGFETHADEQAALNI |
| | | ARSWLFLRSQEYKKYQTNKTTGNTDKRAFVETWQSFYRKKLKEVWKPAV |
| 5 | 515 | QEIKRINKIRRRLVKDSNTKKAGKTGPMKTLLVRVMTPDLRERLENLRKKPENIPQPI |
| | | SNTSRANLNKLLTDYTEMKKAILHVYWEEFQKDPVGLMSRVAQPASKKIDQNKLKPEM |
| | | DEKGNLTTAGFACSQCGQPLFVYKLEQVSEKGKAYTNYFGRCNVAEHEKLILLAQLKP |
| | | EKDSDEAVTYSLGKFGQRALDFYSIHVTKESTHPVKPLAQIAGNRYASGPVGKALSDA |
| | | CMGTIASFLSKYQDIIIEHQKVVKGNQKRLESLRELAGKENLEYPSVTLPPQPHTKEG |
| | | VDAYNEVIARVRMWVNLNLWQKLKLSRDDAKPLLRLKGFPSFPLVERQANEVDWWDMV |
| | | CNVKKLINEKKEDGKVFWQNLAGYKRQEALRPYLSSEEDRKKGKKFARYQLGDLLLHL |
| | | EKKHGEDWGKVYDEAWERIDKKVEGLSKHIKLEEERRSEDAQSKAALTDWLRAKASFV |
| | | IEGLKEADKDEFCRCELKLQKWYGDLRGKPFAIEAENSILDISGFSKQYNCAFIWQKD |
| | | GVKKLNLYLIINYFKGGKLRFKKIKPEAFEANRFYTVINKKSGEIVPMEVNFNFDDPN |
| | | LIILPLAFGKRQGREFIWNDLLSLETGSLKLANGRVIEKTLYNRRTRQDEPALFVALT |
| | | FERREVLDSSNIKPMNLIGVDRGENIPAVIALTDPEGCPLSRFKDSLGNPTHILRIGE |
| | | SYKEKQRTIQAKKEVEQRRAGGYSRKYASKAKNLADDMVRNTARDLLYYAVTQDAMLI |
| | | FENLSRGFGRQGKRTFMAERQYTRMEDWLTAKLAYEGLPSKTYLSKTLAQYTSKTCSN |
| | | CGFTITSADYDRVLEKLKKTATGWMTTINGKELKVEGQITYYNRYKRQNVVKDLSVEL |
| | | DRLSEESVNNDISSWTKGRSGEALSLLKKRFSHRPVQEKFVCLNCGFETHADEQAALN |
| | | IARSWLFLRSQEYKKYQTNKTTGNTDKRAFVETWQSFYRKKLKEVWKPAV |
| 6 | 676 | QEIKRINKIRRRLVKDSNTKKAGKTRGPMKTLLVRVMTPDLRERLENLRKKPENIPQP |
| | | ISNTSRANLNKLLTDYTEMKKAILHVYWEEFQKDPVGLMSRVAQPASKKIDQNKLKPE |
| | | MDEKGNLTTAGFACSQCGQPLFVYKLEQVSEKGKAYTNYFGRCNVAEHEKLIKLAQLK |
| | | PEKDSDEAVTYSLGKFGQRALDFYSIHVTKESTHPVKPLAQIAGNRYASSPVGKALSD |
| | | ACMGTIASFLSKYQDIIIEHQKVVKGNQKRLESLRELAGKENLEYPSVTLPPQPHTKE |
| | | GVDAYNEVIARVRMWVNLNLWQKLKLSRDDAKPLLRLKGFPSFPLVERQANEVDWWDM |
| | | VCNVKKLINEKKEDGKVFWQNLAGYKRQEALRPYLSSEEDRKKGKKFARYQLGDLLLH |
| | | LEKKHGEDWGKVYDEAWERIDKKVEGLSKHIKLEEERRSEDAQSKAALTDWLRAKASF |
| | | VIEGLKEADKDEFCRCELKLQKWYGDLRGKPFAIEAENSILDISGFSKQYNCAFIWQK |
| | | DGVKKLNLYLIINYFKGGKLRFKKIKPEAFEANRFYTVINKKSGEIVPMEVNFNFDDP |
| | | NLIILPLAFGKRQGREFIWNDLLSLETGSLKLANGRVIEKTLYNRRTRQDEPALFVAL |
| | | TFERREVLDSSNIKPMNLIGVDRGENIPAVIALTDPEGCPLSRFKDSLGNPTHILRIG |
| | | ESYKEKQRTIQAKKEVEQRRAGGYSRKYASKAKNLADDMVRNTARDLLYYAVTQDAML |
| | | IFENLSRGFGRQGKRTFMAERQYTRMEDWLTAKLAYEGLPSKTYLSKTLAQYTSKTCS |
| | | NCGFTITSADYDRVLEKLKKTATGWMTTINGKELKVEGQITYYNRYKRQNVVKDLSVE |
| | | LDRLSEESVNNDISSWTKGRSGEALSLLKKRFSHRPVQEKFVCLNCGFETHADEQAAL |
| | | NIARSWLFLRSQEYKKYQTNKTTGNTDKRAFVETWQSFYRKKLKEVWKPAV |
| 7 | 812 | QEIKRINKIRRRLVKDSNTKKAGKTGPMKTLLVRVMTPDLRERLENLRKKPENIPQPI |
| | | SNTSRANLNKLLTDYTEMKKAILHVYWEEFQKDPVGLMSRVAQPASKKIDQNKLKPEM |
| | | DEKGNLTTAGFACSQCGQPLFVYKLEQVSEKGKAYTNYFGRCNVAEHEKLILLAQLKP |
| | | EKDSDEAVTYSLGKFGQRALDFYSIHVTKESTHPVKPLAQIAGNRYASGPVGKALSDA |
| | | CMGTIASFLSKYQDIIIEHQKVVKGNQKRLESLRELAGKENLEYPSVTLPPQPHTKEG |
| | | VDAYNEVIARVRMWVNLNLWQKLKLSRDDAKPLLRLKKFPSFPLVERQANEVDWWDMV |
| | | CNVKKLINEKKEDGKVFWQNLAGYKRQEALRPYLSSEEDRKKGKKFARYQLGDLLLHL |
| | | EKKHGEDWGKVYDEAWERIDKKVEGLSKHIKLEEERRSEDAQSKAALTDWLRAKASFV |
| | | IEGLKEADKDEFCRCELKLQKWYGDLRGKPFAIEAENSILDISGFSKQYNCAFIWQKD |
| | | GVKKLNLYLIINYFKGGKLRFKKIKPEAFEANRFYTVINKKSGEIVPMEVNFNFDDPN |
| | | LIILPLAFGKRQGREFIWNDLLSLETGSLKLANGRVIEKTLYNRRTRQDEPALFVALT |
| | | FERREVLDSSNIKPMNLIGVDRGENIPAVIALTDPEGCPLSRFKDSLGNPTHILRIGE |
| | | SYKEKQRTIQAKKEVEQRRAGGYSRKYASKAKNLADDMVRNTARDLLYYAVTQDAMLI |
| | | FENLSRGFGRQGKRTFMAERQYTRMEDWLTAKLAYEGLPSKTYLSKTLAQYTSKTCSN |
| | | CGFTITSADYDRVLEKLKKTATGWMTTINGKELKVEGQITYYNRYKRQNVVKDLSVEL |
| | | DRLSEESVNNDISSWTKGRSGEALSLLKKRFSHRPVQEKFVCLNCGFETHADEQAALN |
| | | IARSWLFLRSQEYKKYQTNKTTGNTDKRAFVETWQSFYRKKLKEVWKPAV |

Additional engineered CasX proteins contemplated for use in the systems of the disclosure are selected from the group consisting of SEQ ID NOS: 8-342 and 19863-20023, or a sequence having at least about at least about 70%, at least about 75%, at least about 80%, at least about 85%, at least about 90%, at least about 95%, at least about 96%, at least about 97%, at least about 98%, or at least about 99% sequence identity thereto.

In some embodiments, the engineered CasX protein contemplated for use in the systems of the disclosure is a CasX engineered protein comprising an amino acid sequence having two or more modifications relative to a CasX 515 protein of SEQ ID NO: 20024, and wherein the two or more modifications result in an engineered CasX having one or more improved characteristics compared to unmodified parental CasX 515, and wherein the one or more improved characteristics may include an increase in one or more of editing activity, editing specificity, specificity ratio (i.e., the average level of on-targeting editing divided by the average level of off-target editing), editing activity and editing specificity, and editing activity and specificity ratio of the CasX engineered protein. In some embodiments, the improved characteristics are determined compared to the unmodified parental CasX 515 (SEQ ID NO: 20024) in an in vitro assay under comparable conditions.

In some embodiments, the engineered CasX comprises two or more mutations relative to the CasX protein of SEQ ID NO: 20024 (CasX 515), wherein the two mutations are selected from the group consisting of 4.I.G & 64.R.Q, 4.I.G & 169.L.K, 4.I.G & 169.L.Q, 4.I.G & 171.A.D, 4.I.G & 171.A. Y, 4.I.G & 171.A.S, 4.I.G & 224.G.T, 4.I.G & 304.M.T, 4.I.G & 398. Y.T, 4.I.G & 826. V.M, 4.I.G & 887.T.D, 4.I.G & 891.S.Q, 5.-. G & 64.R.Q, 5.-G & 169.L.K, 5.-. G & 169.L.Q, 5.-. G & 171.A.D, 5.-. G & 171.A. Y, 5.-. G & 171.A.S, 5.-. G & 224.G.T, 5.-. G & 304.M.T, 5.-. G & 398.Y.T, 5.-. G & 826. V.M, 5.-G & 887.T.D, 5.-. G & 891.S.Q, 9.K.G & 64.R.Q, 9.K.G & 169.L.K, 9.K.G & 169.L.Q, 9.K.G & 171.A.D, 9.K.G & 171.A.Y, 9.K.G & 171.A.S, 9.K.G & 224.G.T, 9.K.G & 304.M.T, 9.K.G & 398. Y.T, 9.K.G & 826. V.M, 9.K.G & 887.T.D, 9.K.G & 891.S.Q, 27.-.R & 64. R. Q, 27.-.R & 169.L.K, 27.-.R & 169.L.Q, 27.-.R & 171.A.D, 27.-.R & 171.A.Y, 27.-.R & 171.A.S, 27.-.R & 224.G.T, 27.-.R & 304.M.T, 27.-.R & 398. Y.T, 27.-.R & 826. V.M, 27.-.R & 887.T.D, 27.-.R & 891.S.Q. 35.R.P & 64.R.Q, 35.R.P & 169.L.K, 35.R.P & 169.L.Q, 35.R.P & 171.A.D, 35.R.P & 171.A.Y, 35.R.P & 171.A.S, 35.R.P & 224.G.T, 35.R.P & 304.M.T, 35.R.P & 398. Y.T, 35.R.P & 826.V.M, 35.R.P & 887.T.D, 35.R.P & 891.S.Q, 887.T.D & 891.S.Q, 64.R. Q & 169.L.K, 64.R.Q & 169.L.Q, 64.R.Q & 171.A.D, 64.R.Q & 171.A. Y, 64.R.Q & 171.A.S, 64.R.Q & 224.G.T, 64.R.Q & 304.M.T, 64.R.Q & 398. Y.T, 64.R. Q & 826. V.M, 64.R.Q & 887.T.D, 64.R.Q & 891.S.Q, 169.L.K & 171.A.D, 169.L.K & 171.A. Y, 169.L.K & 171.A.S, 169.L.K & 224.G.T, 169.L.K & 304.M.T, 169.L.K & 398. Y.T, 169.L.K & 826. V.M, 169.L.K & 887.T.D, 169.L.K & 891.S.Q, 169.L.Q & 171.A.D, 169.L.Q & 171.A.Y, 169.L.Q & 171.A.S, 169.L.Q & 224.G.T, 169.L.Q & 304.M.T, 169.L.Q & 398. Y.T, 169.L.Q & 826.V.M, 169.L.Q & 887.T.D, 169.L.Q & 891.S.Q, 171.A.D & 224.G.T, 171.A.D & 304.M.T, 171.A.D & 398. Y.T, 171.A.D & 826. V.M, 171.A.D & 887.T.D, 171.A.D & 891.S.Q, 171.A. Y & 224.G.T, 171.A. Y & 304.M.T, 171.A. Y & 398. Y.T, 171.A. Y & 826. V.M, 171.A. Y & 887.T.D, 171.A.Y & 891.S.Q, 171.A.S & 224.G.T, 171.A.S & 304.M.T, 171.A.S & 398. Y.T, 171.A.S & 826. V.M, 171.A.S & 887.T.D, 171.A.S & 891.S.Q, 4.I.G & 35.R.P, 224.G.T & 304.M.T, 224.G.T & 398. Y.T, 224.G.T & 826. V.M, 224.G.T & 887.T.D, 224.G.T & 891.S.Q, 5.-. G & 35.R.P, 4.I.G & 27.-.R, 304.M.T & 398. Y.T, 304.M.T & 826. V.M, 304.M.T & 887.T.D, 304.M.T & 891.S.Q, 9.K.G & 35.R.P, 5.-. G & 27.-.R, 4.I.G & 9.K.G, 398. Y.T & 826. V.M, 398. Y.T & 887.T.D, 398. Y.T & 891.S.Q, 27.-.R & 35.R.P, 9.K.G & 27.-.R, 5.-. G & 9.K.G, 4.I.G & 5.-. G, 826. V.M & 887.T.D, 826. V.M & 891.S.Q, 5.K.G & 27.-.R, 5.K.G & 169.L.K, 5.K.G & 171.A.D, 5.K.G & 304.M.T, 5.K.G & 398. Y.T, 5.K.G & 891.S.Q, 6.-. G & 27.-.R, 6.-. G & 169.L.K, 6.-. G & 171.A.D, 6.-. G & 304.M.T, 6.-. G & 398. Y.T, 6.-. G & 891.S.Q, 304.M. W & 27.-.R, 304.M. W & 169.L.K, 304.M.W & 171.A.D, 304.M. W & 398. Y.T, 304.M. W & 891.S.Q, 481.E.D & 27.-.R, 481.E.D & 169.L.K, 481.E.D & 171.A.D, 481.E.D & 304.M.T, 481.E.D & 398. Y.T, 481.E.D & 891.S.Q, 698.S.R & 27.-.R, 698.S.R & 169.L.K, 698.S.R & 171.A.D, 698.S.R & 304.M.T, 698.S.R & 398.Y.T, and 698.S.R & 891.S.Q, which may be combined in select permutations. In the foregoing, the number refers to the amino acid residue location in the sequence, the first amino acid letter is the original residue and the second is the residue resulting from the mutation, replacing the first residue.

In some embodiments, the engineered CasX protein comprises three mutations in the sequence of CasX 515 (SEQ ID NO: 20024), wherein the three mutations are selected from the group consisting of 27.-.R, 169.L.K, and 329.G.K; 27.-.R, 171.A.D, and 224.G.T; and 35.R.P, 171.A.Y, and 304.M.T, which may be combined in select permutations, and wherein the mutations result in one or more improved characteristics compared to unmodified CasX 515.

Further engineered CasX proteins contemplated for use in the systems of the disclosure are described in International Publication Nos. WO2020247882, WO2022120095, and WO2023235818 which are hereby incorporated by reference in their entirety.

c. CasX Proteins with Domains from Multiple Source Proteins

Also contemplated within the scope of the disclosure are engineered CasX proteins that are chimeric CasX proteins. As used herein, a “chimeric CasX” protein refers to both a CasX protein containing at least two domains from different sources, as well a CasX protein containing at least one domain that itself is chimeric. Accordingly, in some embodiments, a chimeric CasX protein is one that includes at least two domains isolated or derived from different sources, such as from two different naturally occurring CasX proteins, (e.g., from two different CasX reference proteins). In other embodiments, the chimeric CasX protein is one that contains at least one domain that is a chimeric domain, e.g., in some embodiments, part of a domain comprises a substitution from a different CasX protein (from a reference CasX protein, or another engineered CasX protein).

In some embodiments, the helical I-II domain of the engineered CasX protein derived from SEQ ID NO: 2 is replaced with the corresponding helical I-II sequence from SEQ ID NO: 1, resulting in a chimeric CasX protein.

In some embodiments, the helical I-II domain and NTSB domain of the engineered CasX protein derived from SEQ ID NO: 2 is replaced with the corresponding helical I-II and NTSB sequences from SEQ ID NO: 1, resulting in a chimeric CasX protein.

In some embodiments, an engineered CasX protein is a chimeric CasX protein, and comprises at least one chimeric domain. In some embodiments, the at least one chimeric domain can be any of the NTSB, TSL, helical I, helical II, OBD or RuvC domains as described herein. In the case of split or non-contiguous domains such as helical I, RuvC and OBD, a portion of the non-contiguous domain can be replaced with the corresponding portion from any other source.

Domain sequences from reference CasX proteins, and their coordinates, are shown in Table 2. As an example of the foregoing, the chimeric RuvC domain comprises amino acids 660 to 823 of SEQ ID NO: 1 and amino acids 921 to 978 of SEQ ID NO: 2. As an alternative example of the foregoing, a chimeric RuvC domain comprises amino acids 647 to 810 of SEQ ID NO: 2 and amino acids 934 to 986 of SEQ ID NO: 1. In some embodiments, the engineered CasX proteins have a NTSB and a portion of the helical I-II domain derived from the reference CasX sequence of SEQ ID NO: 1, while the other domains are derived from the reference CasX sequence of SEQ ID NO: 2, it being understood that the engineered variants have additional amino acid changes at select locations (relative to the reference sequence), and the resulting chimeric CasX proteins were determined to have improved characteristics relative to the reference CasX proteins. In a particular embodiment, the chimeric helical I domain of the chimeric CasX proteins of SEQ ID NOS: 4-7 comprise amino acids 59-102 of SEQ ID NO: 2, or a sequence having at least about 70%, at least about 80%, at least about 90%, at least about 95%, or at least about 99% sequence identity thereto (helical I-I), and comprises amino acids 192-332 of SEQ ID NO: 1, or at least about 70%, at least about 80%, at least about 90%, at least about 95%, or at least about 99% sequence identity thereto (helical I-II). In one embodiment, chimeric CasX 515 (SEQ ID NO: 5) has an NTSB and the helical I-II domain derived from the reference CasX sequence of SEQ ID NO: 1, and an insertion of P793 relative to the sequence of CasX 491 (SEQ ID NO: 4), wherein the resulting CasX 515 exhibits enhanced specificity for the modification of a target nucleic acid relative to CasX 491. The skilled artisan will understand that the domain boundaries indicated in Table 2 below are approximate, and that protein fragments whose boundaries differ from those given in the table below by 1, 2, or 3 amino acids may have the same activity as the domains described below.

TABLE 2
Domain coordinates in Reference CasX Proteins

| | Coordinates in | Coordinates in |
|---|---|---|
| Domain Name | SEQ ID NO: 1 | SEQ ID NO: 2 |
| OBD-I | 1-55 | 1-57 |
| helical I-I | 56-99 | 58-101 |
| NTSB | 100-190 | 102-191 |
| helical I-II | 191-331 | 192-332 |
| helical II | 332-508 | 333-500 |
| OBD-II | 509-659 | 501-646 |
| RuvC-I | 660-823 | 647-810 |
| TSL | 824-933 | 811-920 |
| RuvC-II | 934-986 | 921-978 |

TABLE 3
Exemplary Domain Sequences in Reference CasX Proteins

| | | |
|---|---|---|
| SEQ ID | Domain | Sequence |

Deltaproteobacter sp. (reference CasX of SEQ ID NO: 1)

| | | |
|---|---|---|
| 343 | OBD-I | EKRINKIRKKLSADNATKPVSRSGPMKTLLVRVMTDDLKKRLEKRRKKPEVMPQ |
| 344 | helical I-I | VISNNAANNLRMLLDDYTKMKEAILQVYWQEFKDDHVGLMCKFA |
| 345 | NTSB | QPASKKIDQNKLKPEMDEKGNLTTAGFACSQCGQPLFVYKLEQVSEKGKAYTNYF |
| | | GRCNVAEHEKLILLAQLKPEKDSDEAVTYSLGKFGQ |
| 346 | helical I-II | RALDFYSIHVTKESTHPVKPLAQIAGNRYASGPVGKALSDACMGTIASFLSKYQD |
| | | IIIEHQKVVKGNQKRLESLRELAGKENLEYPSVTLPPQPHTKEGVDAYNEVIARV |
| | | RMWVNLNLWQKLKLSRDDAKPLLRLKGFPSF |
| 347 | helical II | PVVERRENEVDWWNTINEVKKLIDAKRDMGRVFWSGVTAEKRNTILEGYNYLPNE |
| | | NDHKKREGSLENPKKPAKRQFGDLLLYLEKKYAGDWGKVFDEAWERIDKKIAGLT |
| | | SHIEREEARNAEDAQSKAVLTDWLRAKASFVLERLKEMDEKEFYACEIQLQKWYG |
| | | DLRGNPFAVEAE |
| 348 | OBD-II | NRVVDISGFSIGSDGHSIQYRNLLAWKYLENGKREFYLLMNYGKKGRIRFTDGTD |
| | | IKKSGKWQGLLYGGGKAKVIDLTFDPDDEQLIILPLAFGTRQGREFIWNDLLSLE |
| | | TGLIKLANGRVIEKTIYNKKIGRDEPALFVALTFERREVVD |
| 349 | RuvC-I | PSNIKPVNLIGVDRGENIPAVIALTDPEGCPLPEFKDSSGGPTDILRIGEGYKEK |
| | | QRAIQAAKEVEQRRAGGYSRKFASKSRNLADDMVRNSARDLFYHAVTHDAVLVFE |
| | | NLSRGFGRQGKRTFMTERQYTKMEDWLTAKLAYEGLTSKTYLSKTLAQYTSKTC |
| 350 | TSL | SNCGFTITTADYDGMLVRLKKTSDGWATTLNNKELKAEGQITYYNRYKRQTVEKE |
| | | LSAELDRLSEESGNNDISKWTKGRRDEALFLLKKRFSHRPVQEQFVCLDCGHEVH |
| 351 | RuvC-II | ADEQAALNIARSWLFLNSNSTEFKSYKSGKQPFVGAWQAFYKRRLKEVWKPNA |

Planctomycetes sp. (Reference CasX of SEQ ID NO: 2)

| | | |
|---|---|---|
| 352 | OBD-I | QEIKRINKIRRRLVKDSNTKKAGKTGPMKTLLVRVMTPDLRERLENLRKKPENIP |
| | | Q |
| 353 | helical I-I | PISNTSRANLNKLLTDYTEMKKAILHVYWEEFQKDPVGLMSRVA |
| 354 | NTSB | QPAPKNIDQRKLIPVKDGNERLTSSGFACSQCCQPLYVYKLEQVNDKGKPHTNYF |
| | | GRCNVSEHERLILLSPHKPEANDELVTYSLGKFGQ |
| 355 | helical I-II | RALDFYSIHVTRESNHPVKPLEQIGGNSCASGPVGKALSDACMGAVASFLTKYQD |
| | | IILEHQKVIKKNEKRLANLKDIASANGLAFPKITLPPQPHTKEGIEAYNNVVAQI |
| | | VIWVNLNLWQKLKIGRDEAKPLQRLKGFPSF |
| 356 | helical II | PLVERQANEVDWWDMVCNVKKLINEKKEDGKVFWQNLAGYKRQEALLPYLSSEED |
| | | RKKGKKFARYQFGDLLLHLEKKHGEDWGKVYDEAWERIDKKVEGLSKHIKLEEER |
| | | RSEDAQSKAALTDWLRAKASFVIEGLKEADKDEFCRCELKLQKWYGDLRGKPFAI |
| | | EAE |
| 357 | OBD-II | NSILDISGFSKQYNCAFIWQKDGVKKLNLYLIINYFKGGKLRFKKIKPEAFEANR |
| | | FYTVINKKSGEIVPMEVNFNFDDPNLIILPLAFGKRQGREFIWNDLLSLETGSLK |
| | | LANGRVIEKTLYNRRTRQDEPALFVALTFERREVLD |
| 358 | RuvC-I | SSNIKPMNLIGIDRGENIPAVIALTDPEGCPLSRFKDSLGNPTHILRIGESYKEK |
| | | QRTIQAAKEVEQRRAGGYSRKYASKAKNLADDMVRNTARDLLYYAVTQDAMLIFE |
| | | NLSRGFGRQGKRTFMAERQYTRMEDWLTAKLAYEGLPSKTYLSKTLAQYTSKTC |
| 359 | TSL | SNCGFTITSADYDRVLEKLKKTATGWMTTINGKELKVEGQITYYNRYKRQNVVKD |
| | | LSVELDRLSEESVNNDISSWTKGRSGEALSLLKKRFSHRPVQEKFVCLNCGFETH |
| 360 | RuvC-II | ADEQAALNIARSWLFLRSQEYKKYQTNKTTGNTDKRAFVETWQSFYRKKLKEVWK |
| | | PAV |

d. CasX Fusion Proteins

Also contemplated within the scope of the disclosure are engineered CasX proteins comprising a heterologous protein (i.e., a protein not derived from a CasX) fused to the CasX for use in the systems of the disclosure. This includes engineered CasX comprising N-terminal or C-terminal fusions of the CasX to a heterologous protein or domain thereof. In some embodiments, the engineered CasX protein is fused to one or more proteins or domains thereof that has a different activity of interest, resulting in a fusion protein.

In some cases, a heterologous polypeptide (a fusion partner) for use with an engineered CasX protein in the systems of the disclosure provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES) to escort the engineered CasX protein through the nuclear pore complex, a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject engineered CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, an engineered CasX protein is fused to one or more nuclear localization signals (NLS). The disclosure contemplates assembly of multiple NLS in various configurations for linkage to the engineered CasX protein utilized in the embodiments described herein. In some embodiments, a single NLS is linked at or near (e.g., within 20 amino acids of) the N-terminus of the engineered CasX protein. In some embodiments, a single NLS is linked at or near the N-terminus and/or at or near the C-terminus of the engineered CasX protein. In some embodiments, the N-terminal NLS comprises one or more a c-MYC NLS. In some embodiments, the C-terminal NLS comprises one or more c-MYC NLS. In some embodiments, 2, 3, 4 or more NLS are linked by linker peptides at or near the C-terminus and/or the N-terminus of the engineered CasX protein. The person of ordinary skill in the art will understand that an NLS at or near the N- or C-terminus of a protein can be within 1, 2, 3, 4, 5, 6, 7, 8, 9, 10, 11, 12, 13, 14, 15, 16, 17, 18, 19 or 20 amino acids of the N- or C-terminus. In some embodiments, the NLS linked to the N-terminus of the engineered CasX protein are identical to the NLS linked to the C-terminus. In other embodiments, the NLS linked to the N-terminus of the engineered CasX protein are different to the NLS linked to the C-terminus. In some cases, non-limiting examples of NLSs suitable for use with an engineered CasX protein in the systems of the disclosure include sequences having at least about 80%, at least about 90%, or at least about 95% identity or are identical to sequences derived from: the NLS of the SV40 virus large T-antigen, having the amino acid sequence PKKKRKV (SEQ ID NO: 361); the NLS from nucleoplasmin (e.g., the nucleoplasmin bipartite NLS with the sequence KRPAATKKAGQAKKKK (SEQ ID NO: 362); the c-MYC NLS having the amino acid sequence PAAKRVKLD (SEQ ID NO: 363) or RQRRNELKRSP (SEQ ID NO: 364). In some embodiments, the NLS linked to the N-terminus of the engineered CasX protein is selected from the group consisting of the N-terminal sequences as set forth in Table 4. In some embodiments, the NLS linked to the C-terminus of the CasX protein is selected from the group consisting of the C-terminal sequences as set forth in Table 5. In some embodiments, NLSs suitable for use with an engineered CasX in the systems of the disclosure include sequences having at least about 80%, at least about 90%, or at least about 95% identity or are identical to one or more sequences of Table 5. The skilled artisan will understand that the disclosure of NLS sequences in Tables 4 and 5 as N- or C-terminal, respectively, is exemplary only. Any of the NLS sequences disclosed in Table 4 or 5 may be located at or near the N or C terminus, or both.

TABLE 4
N-terminal NLS Amino Acid Sequences

| NLS Amino Acid Sequence* | SEQ ID NO |
|---|---|
| PKKKRKVSR | 365 |
| PKKKRKVGGSPKKKRKVGGSPKKKRKVGGSPKKKRKVSR | 366 |
| PKKKRKVGGSPKKKRKVGGSPKKKRKVGGSPKKKRKVGGSPKKKRKVGGSPKKKRKVSR | 367 |
| PAAKRVKLDSR | 368 |
| PAAKRVKLDGGSPAAKRVKLDSR | 369 |
| PAAKRVKLDGGSPAAKRVKLDGGSPAAKRVKLDGGSPAAKRVKLDSR | 370 |
| PAAKRVKLDGGSPAAKRVKLDGGSPAAKRVKLDGGSPAAKRVKLDGGSPAAKRVKLDGGS | 371 |
| PAAKRVKLDSR | |
| KRPAATKKAGQAKKKKSR | 372 |
| KRPAATKKAGQAKKKKGGSKRPAATKKAGQAKKKKSR | 373 |
| PAAKRVKLDGGSPKKKRKVSR | 374 |
| PAAKKKKLDGGSPKKKRKVSR | 375 |
| PAAKKKKLDSR | 376 |
| PAAKKKKLDGGSPAAKKKKLDGGSPAAKKKKLDSR | 377 |
| PAAKKKKLDGGSPAAKKKKLDGGSPAAKKKKLDGGSPAAKKKKLDSR | 378 |
| PAKRARRGYKCSR | 379 |
| PAKRARRGYKCGSPAKRARRGYKCSR | 380 |
| PRRKREESR | 381 |
| PYRGRKESR | 382 |
| PLRKRPRRSR | 383 |
| PLRKRPRRGSPLRKRPRRSR | 384 |
| PAAKRVKLDGGKRTADGSEFESPKKKRKVGGS | 385 |
| PAAKRVKLDGGKRTADGSEFESPKKKRKVPPPPG | 386 |
| PAAKRVKLDGGKRTADGSEFESPKKKRKVGIHGVPAAPG | 387 |
| PAAKRVKLDGGKRTADGSEFESPKKKRKVGGGSGGGSPG | 388 |
| PAAKRVKLDGGKRTADGSEFESPKKKRKVPGGGSGGGSPG | 389 |
| PAAKRVKLDGGKRTADGSEFESPKKKRKVAEAAAKEAAAKEAAAKAPG | 390 |
| PAAKRVKLDGGSPKKKRKVGGS | 391 |
| PAAKRVKLDPPPPKKKRKVPG | 392 |
| PAAKRVKLDPG | 393 |
| PAAKRVKLDGGGSGGGSGGGS | 394 |
| PAAKRVKLDPPP | 395 |
| PAAKRVKLDGGGSGGGSGGGSPPP | 396 |
| PKKKRKVPPP | 397 |
| PKKKRKVGGS | 398 |

*Residues in bold are NLS residues, while unbolded residues are linkers.

TABLE 5
C-terminal NLS Amino Acid Sequences

| NLS Amino Acid Sequence | SEQ ID NO |
|---|---|
| GSPKKKRKVGGSPKKKRKVGGSPKKKRKVGGSPKKKRKV | 399 |
| GSPKKKRKVGGSPKKKRKVGGSPKKKRKVGGSPKKKRKVGGSPKKKRKVGGSPKKKRKV | 400 |
| GSPAAKRVKLDGGSPAAKRVKLD | 401 |
| GSPAAKRVKLDGGSPAAKRVKLDGGSPAAKRVKLDGGSPAAKRVKLD | 402 |
| GSKRPAATKKAGQAKKKK | 403 |
| KRPAATKKAGQAKKKKGGSKRPAATKKAGQAKKKK | 404 |
| GSKLGPRKATGRWGS | 405 |
| GSKRKGSPERGERKRHWGS | 406 |
| GSPKKKRKVGSGSKRPAATKKAGQAKKKKLE | 407 |
| GPKRTADSQHSTPPKTKRKVEFEPKKKRKV | 408 |
| GGGSGGGSKRTADSQHSTPPKTKRKVEFEPKKKRKV | 409 |
| AEAAAKEAAAKEAAAKAKRTADSQHSTPPKTKRKVEFEPKKKRKV | 410 |
| GPPKKKRKVGGSKRTADSQHSTPPKTKRKVEFEPKKKRKV | 411 |
| GPAEAAAKEAAAKEAAAKAPAAKRVKLD | 412 |
| GPGGGSGGGSGGGSPAAKRVKLD | 413 |
| GPPAAKRVKLD | 414 |
| VGSKRPAATKKAGQAKKKK | 415 |
| TGGGPGGGAAAGSGSPKKKRKVGSGSKRPAATKKAGQAKKKKLE | 416 |
| TGGGPGGGAAAGSGSPKKKRKVGSGS | 417 |
| PPPPKKKRKVPPP | 418 |
| GGSPKKKRKVPPP | 419 |
| PPPPKKKRKV | 420 |
| GGSPKKKRKV | 421 |
| GGSPKKKRKVGGSGGSGGS | 422 |
| GGSPKKKRKVGGSPKKKRKV | 423 |
| GGSGGSGGSPKKKRKVGGSPKKKRKV | 424 |
| VGGGSGGGSGGGSPAAKRVKLD | 425 |
| VPPPPAAKRVKLD | 426 |
| VPPPGGGSGGGSGGGSPAAKRVKLD | 427 |
| VGSPAAKRVKLD | 428 |

In some embodiments, the one or more NLSs are linked to the engineered CasX protein or to adjacent NLS with a linker peptide. In some embodiments, the linker peptide is selected from the group consisting of SR, RS, TS, GS, GGS, PPP, GGS, VGS, (G) n (SEQ ID NO: 429), (GS) n (SEQ ID NO: 430), (GSGGS) n (SEQ ID NO: 431), (GGSGGS) n (SEQ ID NO: 432), (GGGS) n (SEQ ID NO: 433), GGSG (SEQ ID NO: 434), GGSGG (SEQ ID NO: 435), GSGSG (SEQ ID NO: 436), GSGGG (SEQ ID NO: 437), GGGSG (SEQ ID NO: 438), GSSSG (SEQ ID NO: 439), GPGP (SEQ ID NO: 440), GGP, PPP, PPAPPA (SEQ ID NO: 441), PPPG (SEQ ID NO: 442), PPPGPPP (SEQ ID NO: 443), PPP (GGGS) n (SEQ ID NO: 444), (GGGS) nPPP (SEQ ID NO: 445), AEAAAKEAAAKEAAAKA (SEQ ID NO: 446), TPPKTKRKVEFE (SEQ ID NO: 447), GGSGGGS (SEQ ID NO: 448), GSGSGGG (SEQ ID NO: 449), and SSGNSNANSRGPSFSSGLVPLSLRGSH (SEQ ID NO: 450), where n is 1 to 5.

In general, NLS (or multiple NLSs) are of sufficient strength to drive accumulation of an engineered CasX fusion protein in the nucleus of a eukaryotic cell. Detection of accumulation in the nucleus may be performed by any suitable technique. For example, a detectable marker may be fused to an engineered CasX fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

e. Sequences Encoding Engineered CasX Proteins

Provided herein are nucleic acids, such as mRNA and DNA, comprising sequences encoding engineered CasX proteins of the disclosure. In some embodiments, the sequences encoding CasX proteins were generated based using one or more parameters. Non-limiting examples of such parameters include the codon usage in human host cells (e.g., utilizing the codon adaptation index (CAI)) or codon-usage tables derived from biologics intended for use as therapeutics. In some embodiments, provided herein are sequences encoding CasX proteins that are codon-optimized for expression in a human cell.

The disclosure provides an mRNA sequence encoding CasX 491 (SEQ ID NO: 4), or a sequence having at least about 70%, at least about 80%, at least about 85%, at least about 90%, at least about 95%, at least about 96%, at least about 97%, at least about 98%, or having at least about 99% sequence identity thereto. In some embodiments, the mRNA sequence encoding CasX 491 comprises the nucleic acid sequence of SEQ ID NO: 19699, or a sequence having at least about 50%, at least about 60%, at least about 70%, at least about 80%, at least about 85%, at least about 90%, at least about 95%, at least about 96%, at least about 97%, at least about 98%, or having at least about 99% sequence identity thereto.

The disclosure provides an mRNA sequence encoding CasX 515 (SEQ ID NO: 5), or a sequence having at least about 70%, at least about 80%, at least about 85%, at least about 90%, at least about 95%, at least about 96%, at least about 97%, at least about 98%, or having at least about 99% sequence identity thereto. In some embodiments, the mRNA sequence encoding CasX 515 comprises the nucleic acid sequence of SEQ ID NO: 19700, or a sequence having at least about 50%, at least about 60%, at least about 70%, at least about 80%, at least about 85%, at least about 90%, at least about 95%, at least about 96%, at least about 97%, at least about 98%, or having at least about 99% sequence identity thereto.

The disclosure provides an mRNA sequence encoding CasX 676 (SEQ ID NO: 6), or a sequence having at least about 70%, at least about 80%, at least about 85%, at least about 90%, at least about 95%, at least about 96%, at least about 97%, at least about 98%, or having at least about 99% sequence identity thereto. In some embodiments, the mRNA sequence encoding CasX 676 comprises the nucleic acid sequence of SEQ ID NO: 19701, or a sequence having at least about 50%, at least about 60%, at least about 70%, at least about 80%, at least about 85%, at least about 90%, at least about 95%, at least about 96%, at least about 97%, at least about 98%, or having at least about 99% sequence identity thereto.

The disclosure provides an mRNA sequence encoding CasX 812 (SEQ ID NO: 7), or a sequence having at least about 70%, at least about 80%, at least about 85%, at least about 90%, at least about 95%, at least about 96%, at least about 97%, at least about 98%, or having at least about 99% sequence identity thereto. In some embodiments, the mRNA sequence encoding CasX 812 comprises the nucleic acid sequence of SEQ ID NO: 19702, or a sequence having at least about 50%, at least about 60%, at least about 70%, at least about 80%, at least about 85%, at least about 90%, at least about 95%, at least about 96%, at least about 97%, at least about 98%, or having at least about 99% sequence identity thereto.

Exemplary sequences encoding engineered CasX, including RNA and DNA sequences encoding engineered CasX, are provided as SEQ ID NOS: 19699-19702,19735, 19751, 19752, and 19816-19820 of Table 6. The skilled artisan will appreciate that, for the DNA and RNA sequences such as those disclosed in Table 6, if the sequence is a DNA sequence, T can be substituted by U to generate the corresponding RNA. Similarly, for RNA sequences, U can be substituted with T to generate the corresponding DNA sequence.

TABLE 6
RNA and DNA sequences encoding CasX

| SEQ | CasX | |
|---|---|---|
| ID NO: | Designation | Sequence* |
| 19699 | 491 | CAGGAGAUCAAGCGGAUUAAUAAAAUUCGGAGAAGACUGGUGAAGGAUUCUAA |
| | | CACAAAGAAGGCUGGCAAGACAGGCCCUAUGAAGACACUGCUGGUGAGAGUGA |
| | | UGACACCCGACCUGAGAGAAAGACUGGAAAACCUGAGAAAGAAGCCUGAGAAU |
| | | AUCCCCCAGCCCAUCAGCAACACAAGCCGGGCCAACCUGAAUAAGCUGCUGAC |
| | | CGACUACACCGAAAUGAAGAAGGCCAUCCUGCACGUGUAUUGGGAAGAGUUCC |
| | | AGAAAGACCCAGUCGGCCUGAUGAGCAGAGUGGCUCAGCCUGCCAGCAAGAAG |
| | | AUCGAUCAGAACAAGCUGAAGCCCGAAAUGGACGAGAAGGGGAACCUGACAAC |
| | | CGCCGGCUUUGCCUGUAGCCAGUGCGGCCAGCCCCUGUUUGUGUACAAACUGG |
| | | AACAGGUGAGCGAAAAGGGCAAGGCUUACACGAAUUACUUCGGCAGAUGCAAC |
| | | GUGGCCGAGCACGAGAAGCUGAUCCUGCUGGCCCAGCUGAAGCCUGAGAAGGA |
| | | UAGCGAUGAGGCAGUGACAUAUUCCCUGGGCAAGUUCGGACAGCGGGCCCUGG |
| | | AUUUUUAUUCCAUUCAUGUGACCAAGGAAUCCACCCACCCCGUCAAGCCUCUU |
| | | GCCCAAAUUGCCGGCAACAGAUACGCCAGCGGCCCCGUGGGCAAGGCCCUGAG |
| | | CGACGCCUGUAUGGGCACCAUCGCCAGCUUCCUGUCUAAGUACCAGGACAUUA |
| | | UCAUCGAGCACCAGAAGGUGGUGAAGGGCAACCAGAAGAGACUGGAGAGCCUG |
| | | CGCGAGCUGGCCGGCAAGGAAAACCUGGAGUAUCCUAGCGUGACCCUGCCUCC |
| | | UCAGCCUCAUACAAAGGAGGGCGUGGAUGCCUACAACGAAGUGAUCGCCCGGG |
| | | UGCGGAUGUGGGUGAACCUGAAUCUGUGGCAGAAGCUGAAGCUGUCUAGAGAC |
| | | GACGCCAAGCCCCUGCUGAGACUGAAGGGCUUCCCCAGCUUCCCUCUGGUGGA |
| | | GAGACAGGCAAAUGAAGUGGACUGGUGGGACAUGGUGUGUAACGUGAAGAAGC |
| | | UGAUCAAUGAGAAGAAGGAGGACGGCAAAGUGUUCUGGCAGAAUCUGGCCGGC |
| | | UACAAGCGUCAGGAGGCCCUGCGGCCCUACCUGAGCAGCGAGGAAGACAGAAA |
| | | GAAGGGCAAGAAGUUCGCCCGGUAUCAGCUGGGGGACCUGCUGCUGCACCUCG |
| | | AGAAGAAGCACGGCGAAGACUGGGGGAAGGUGUACGAUGAGGCCUGGGAGCGG |
| | | AUCGAUAAGAAGGUGGAGGGCCUGAGCAAGCACAUCAAGCUGGAGGAGGAACG |
| | | GAGAUCUGAGGACGCCCAGAGCAAGGCCGCCCUGACCGACUGGCUGAGAGCCA |
| | | AGGCCAGCUUCGUCAUCGAGGGGCUGAAGGAGGCCGACAAGGACGAGUUCUGC |
| | | CGGUGCGAACUGAAGCUGCAGAAGUGGUACGGAGAUCUGAGAGGCAAACCUUU |
| | | CGCCAUCGAGGCCGAGAACAGCAUCCUGGACAUCAGCGGCUUCAGCAAGCAGU |
| | | ACAACUGCGCCUUUAUUUGGCAGAAGGACGGAGUGAAGAAGCUGAACCUGUAC |
| | | CUGAUCAUCAACUAUUUCAAGGGCGGCAAGCUGAGAUUCAAGAAGAUCAAGCC |
| | | UGAAGCCUUCGAGGCCAACAGAUUCUACACCGUGAUUAACAAGAAAAGCGGAG |
| | | AGAUCGUGCCAAUGGAAGUGAACUUCAACUUCGACGACCCUAACCUGAUCAUC |
| | | CUGCCCCUGGCAUUUGGCAAGCGGCAGGGCAGAGAGUUCAUCUGGAACGACCU |
| | | GCUGUCUCUGGAGACCGGCAGCCUGAAGCUGGCCAACGGCAGAGUGAUCGAGA |
| | | AGACACUGUACAACAGACGAACCAGACAAGACGAGCCCGCCCUGUUUGUGGCC |
| | | CUGACCUUCGAGAGAAGAGAGGUGCUGGACAGCAGCAAUAUCAAGCCUAUGAA |
| | | CCUGAUCGGCGUGGACCGGGGCGAGAACAUCCCUGCCGUGAUCGCCCUUACCG |
| | | ACCCCGAGGGAUGCCCUCUGAGCCGGUUUAAAGACAGCCUGGGCAACCCUACC |
| | | CACAUCCUGAGAAUUGGCGAGUCCUACAAGGAGAAGCAGAGAACCAUCCAGGC |
| | | CAAGAAGGAGGUGGAGCAGCGGCGGGCUGGCGGCUACUCCCGGAAGUACGCCA |
| | | GCAAGGCCAAGAACCUGGCCGACGACAUGGUUAGAAAUACCGCCAGAGACCUC |
| | | CUGUACUACGCUGUGACCCAGGACGCCAUGCUGAUCUUCGAGAACCUGAGCAG |
| | | AGGCUUCGGCAGACAGGGCAAGAGAACCUUCAUGGCCGAGAGACAGUACACCC |
| | | GGAUGGAGGACUGGCUGACCGCCAAGCUGGCCUACGAGGGCCUGUCUAAGACC |
| | | UACCUGUCCAAGACCUUGGCACAGUACACCAGCAAGACAUGCUCUAACUGCGG |
| | | CUUCACAAUCACGAGCGCCGACUACGACCGGGUGCUGGAGAAACUGAAGAAGA |
| | | CCGCCACAGGCUGGAUGACCACCAUUAACGGCAAGGAGCUGAAGGUGGAGGGC |
| | | CAGAUCACCUACUACAACAGGUACAAACGGCAGAACGUGGUGAAGGACCUGAG |
| | | CGUGGAACUGGAUAGACUGAGCGAGGAAAGCGUAAACAAUGACAUCAGCAGCU |
| | | GGACCAAGGGCCGGAGCGGCGAGGCCCUGAGCCUGCUGAAGAAGAGAUUCUCC |
| | | CACAGACCAGUGCAGGAGAAGUUCGUGUGUCUGAACUGCGGCUUCGAGACCCA |
| | | CGCCGACGAGCAAGCCGCCCUGAACAUCGCCCGGUCUUGGCUUUUCCUGCGGA |
| | | GCCAGGAGUACAAGAAGUACCAGACAAACAAGACCACAGGCAACACAGACAAG |
| | | AGAGCCUUCGUCGAGACCUGGCAGAGCUUCUACAGAAAGAAGCUGAAGGAGGU |
| | | GUGGAAGCCUGCCGUG |
| 19700 | 515 | CAGGAGAUCAAGCGGAUUAAUAAAAUUCGGAGAAGACUGGUGAAGGAUUCUAA |
| | | CACAAAGAAGGCUGGCAAGACAGGCCCUAUGAAGACACUGCUGGUGAGAGUGA |
| | | UGACACCCGACCUGAGAGAAAGACUGGAAAACCUGAGAAAGAAGCCUGAGAAU |
| | | AUCCCCCAGCCCAUCAGCAACACAAGCCGGGCCAACCUGAAUAAGCUGCUGAC |
| | | CGACUACACCGAAAUGAAGAAGGCCAUCCUGCACGUGUAUUGGGAAGAGUUCC |
| | | AGAAAGACCCAGUCGGCCUGAUGAGCAGAGUGGCUCAGCCUGCCAGCAAGAAG |
| | | AUCGAUCAGAACAAGCUGAAGCCCGAAAUGGACGAGAAGGGGAACCUGACAAC |
| | | CGCCGGCUUUGCCUGUAGCCAGUGCGGCCAGCCCCUGUUUGUGUACAAACUGG |
| | | AACAGGUGAGCGAAAAGGGCAAGGCUUACACGAAUUACUUCGGCAGAUGCAAC |
| | | GUGGCCGAGCACGAGAAGCUGAUCCUGCUGGCCCAGCUGAAGCCUGAGAAGGA |
| | | UAGCGAUGAGGCAGUGACAUAUUCCCUGGGCAAGUUCGGACAGCGGGCCCUGG |
| | | AUUUUUAUUCCAUUCAUGUGACCAAGGAAUCCACCCACCCCGUCAAGCCUCUU |
| | | GCCCAAAUUGCCGGCAACAGAUACGCCAGCGGCCCCGUGGGCAAGGCCCUGAG |
| | | CGACGCCUGUAUGGGCACCAUCGCCAGCUUCCUGUCUAAGUACCAGGACAUUA |
| | | UCAUCGAGCACCAGAAGGUGGUGAAGGGCAACCAGAAGAGACUGGAGAGCCUG |
| | | CGCGAGCUGGCCGGCAAGGAAAACCUGGAGUAUCCUAGCGUGACCCUGCCUCC |
| | | UCAGCCUCAUACAAAGGAGGGCGUGGAUGCCUACAACGAAGUGAUCGCCCGGG |
| | | UGCGGAUGUGGGUGAACCUGAAUCUGUGGCAGAAGCUGAAGCUGUCUAGAGAC |
| | | GACGCCAAGCCCCUGCUGAGACUGAAGGGCUUCCCCAGCUUCCCUCUGGUGGA |
| | | GAGACAGGCAAAUGAAGUGGACUGGUGGGACAUGGUGUGUAACGUGAAGAAGC |
| | | UGAUCAAUGAGAAGAAGGAGGACGGCAAAGUGUUCUGGCAGAAUCUGGCCGGC |
| | | UACAAGCGUCAGGAGGCCCUGCGGCCCUACCUGAGCAGCGAGGAAGACAGAAA |
| | | GAAGGGCAAGAAGUUCGCCCGGUAUCAGCUGGGGGACCUGCUGCUGCACCUCG |
| | | AGAAGAAGCACGGCGAAGACUGGGGGAAGGUGUACGAUGAGGCCUGGGAGCGG |
| | | AUCGAUAAGAAGGUGGAGGGCCUGAGCAAGCACAUCAAGCUGGAGGAGGAACG |
| | | GAGAUCUGAGGACGCCCAGAGCAAGGCCGCCCUGACCGACUGGCUGAGAGCCA |
| | | AGGCCAGCUUCGUCAUCGAGGGGCUGAAGGAGGCCGACAAGGACGAGUUCUGC |
| | | CGGUGCGAACUGAAGCUGCAGAAGUGGUACGGAGAUCUGAGAGGCAAACCUUU |
| | | CGCCAUCGAGGCCGAGAACAGCAUCCUGGACAUCAGCGGCUUCAGCAAGCAGU |
| | | ACAACUGCGCCUUUAUUUGGCAGAAGGACGGAGUGAAGAAGCUGAACCUGUAC |
| | | CUGAUCAUCAACUAUUUCAAGGGCGGCAAGCUGAGAUUCAAGAAGAUCAAGCC |
| | | UGAAGCCUUCGAGGCCAACAGAUUCUACACCGUGAUUAACAAGAAAAGCGGAG |
| | | AGAUCGUGCCAAUGGAAGUGAACUUCAACUUCGACGACCCUAACCUGAUCAUC |
| | | CUGCCCCUGGCAUUUGGCAAGCGGCAGGGCAGAGAGUUCAUCUGGAACGACCU |
| | | GCUGUCUCUGGAGACCGGCAGCCUGAAGCUGGCCAACGGCAGAGUGAUCGAGA |
| | | AGACACUGUACAACAGACGAACCAGACAAGACGAGCCCGCCCUGUUUGUGGCC |
| | | CUGACCUUCGAGAGAAGAGAGGUGCUGGACAGCAGCAAUAUCAAGCCUAUGAA |
| | | CCUGAUCGGCGUGGACCGGGGCGAGAACAUCCCUGCCGUGAUCGCCCUUACCG |
| | | ACCCCGAGGGAUGCCCUCUGAGCCGGUUUAAAGACAGCCUGGGCAACCCUACC |
| | | CACAUCCUGAGAAUUGGCGAGUCCUACAAGGAGAAGCAGAGAACCAUCCAGGC |
| | | CAAGAAGGAGGUGGAGCAGCGGCGGGCUGGCGGCUACUCCCGGAAGUACGCCA |
| | | GCAAGGCCAAGAACCUGGCCGACGACAUGGUUAGAAAUACCGCCAGAGACCUC |
| | | CUGUACUACGCUGUGACCCAGGACGCCAUGCUGAUCUUCGAGAACCUGAGCAG |
| | | AGGCUUCGGCAGACAGGGCAAGAGAACCUUCAUGGCCGAGAGACAGUACACCC |
| | | GGAUGGAGGACUGGCUGACCGCCAAGCUGGCCUACGAGGGCCUGCCCUCUAAG |
| | | ACCUACCUGUCCAAGACCUUGGCACAGUACACCAGCAAGACAUGCUCUAACUG |
| | | CGGCUUCACAAUCACGAGCGCCGACUACGACCGGGUGCUGGAGAAACUGAAGA |
| | | AGACCGCCACAGGCUGGAUGACCACCAUUAACGGCAAGGAGCUGAAGGUGGAG |
| | | GGCCAGAUCACCUACUACAACAGGUACAAACGGCAGAACGUGGUGAAGGACCU |
| | | GAGCGUGGAACUGGAUAGACUGAGCGAGGAAAGCGUAAACAAUGACAUCAGCA |
| | | GCUGGACCAAGGGCCGGAGCGGCGAGGCCCUGAGCCUGCUGAAGAAGAGAUUC |
| | | UCCCACAGACCAGUGCAGGAGAAGUUCGUGUGUCUGAACUGCGGCUUCGAGAC |
| | | CCACGCCGACGAGCAAGCCGCCCUGAACAUCGCCCGGUCUUGGCUUUUCCUGC |
| | | GGAGCCAGGAGUACAAGAAGUACCAGACAAACAAGACCACAGGCAACACAGAC |
| | | AAGAGAGCCUUCGUCGAGACCUGGCAGAGCUUCUACAGAAAGAAGCUGAAGGA |
| | | GGUGUGGAAGCCUGCCGUG |
| 19701 | 676 | CAGGAGAUCAAGCGGAUUAAUAAAAUUCGGAGAAGACUGGUGAAGGAUUCUAA |
| | | CACAAAGAAGGCUGGCAAGACACGGGGCCCUAUGAAGACACUGCUGGUGAGAG |
| | | UGAUGACACCCGACCUGAGAGAAAGACUGGAAAACCUGAGAAAGAAGCCUGAG |
| | | AAUAUCCCCCAGCCCAUCAGCAACACAAGCCGGGCCAACCUGAAUAAGCUGCU |
| | | GACCGACUACACCGAAAUGAAGAAGGCCAUCCUGCACGUGUAUUGGGAAGAGU |
| | | UCCAGAAAGACCCAGUCGGCCUGAUGAGCAGAGUGGCUCAGCCUGCCAGCAAG |
| | | AAGAUCGAUCAGAACAAGCUGAAGCCCGAAAUGGACGAGAAGGGGAACCUGAC |
| | | AACCGCCGGCUUUGCCUGUAGCCAGUGCGGCCAGCCCCUGUUUGUGUACAAAC |
| | | UGGAACAGGUGAGCGAAAAGGGCAAGGCUUACACGAAUUACUUCGGCAGAUGC |
| | | AACGUGGCCGAGCACGAGAAGCUGAUCAAGCUGGCCCAGCUGAAGCCUGAGAA |
| | | GGAUAGCGAUGAGGCAGUGACAUAUUCCCUGGGCAAGUUCGGACAGCGGGCCC |
| | | UGGAUUUUUAUUCCAUUCAUGUGACCAAGGAAUCCACCCACCCCGUCAAGCCU |
| | | CUUGCCCAAAUUGCCGGCAACAGAUACGCCUCCAGCCCCGUGGGCAAGGCCCU |
| | | GAGCGACGCCUGUAUGGGCACCAUCGCCAGCUUCCUGUCUAAGUACCAGGACA |
| | | UUAUCAUCGAGCACCAGAAGGUGGUGAAGGGCAACCAGAAGAGACUGGAGAGC |
| | | CUGCGCGAGCUGGCCGGCAAGGAAAACCUGGAGUAUCCUAGCGUGACCCUGCC |
| | | UCCUCAGCCUCAUACAAAGGAGGGCGUGGAUGCCUACAACGAAGUGAUCGCCC |
| | | GGGUGCGGAUGUGGGUGAACCUGAAUCUGUGGCAGAAGCUGAAGCUGUCUAGA |
| | | GACGACGCCAAGCCCCUGCUGAGACUGAAGGGCUUCCCCAGCUUCCCUCUGGU |
| | | GGAGAGACAGGCAAAUGAAGUGGACUGGUGGGACAUGGUGUGUAACGUGAAGA |
| | | AGCUGAUCAAUGAGAAGAAGGAGGACGGCAAAGUGUUCUGGCAGAAUCUGGCC |
| | | GGCUACAAGCGUCAGGAGGCCCUGCGGCCCUACCUGAGCAGCGAGGAAGACAG |
| | | AAAGAAGGGCAAGAAGUUCGCCCGGUAUCAGCUGGGGGACCUGCUGCUGCACC |
| | | UCGAGAAGAAGCACGGCGAAGACUGGGGGAAGGUGUACGAUGAGGCCUGGGAG |
| | | CGGAUCGAUAAGAAGGUGGAGGGCCUGAGCAAGCACAUCAAGCUGGAGGAGGA |
| | | ACGGAGAUCUGAGGACGCCCAGAGCAAGGCCGCCCUGACCGACUGGCUGAGAG |
| | | CCAAGGCCAGCUUCGUCAUCGAGGGGCUGAAGGAGGCCGACAAGGACGAGUUC |
| | | UGCCGGUGCGAACUGAAGCUGCAGAAGUGGUACGGAGAUCUGAGAGGCAAACC |
| | | UUUCGCCAUCGAGGCCGAGAACAGCAUCCUGGACAUCAGCGGCUUCAGCAAGC |
| | | AGUACAACUGCGCCUUUAUUUGGCAGAAGGACGGAGUGAAGAAGCUGAACCUG |
| | | UACCUGAUCAUCAACUAUUUCAAGGGCGGCAAGCUGAGAUUCAAGAAGAUCAA |
| | | GCCUGAAGCCUUCGAGGCCAACAGAUUCUACACCGUGAUUAACAAGAAAAGCG |
| | | GAGAGAUCGUGCCAAUGGAAGUGAACUUCAACUUCGACGACCCUAACCUGAUC |
| | | AUCCUGCCCCUGGCAUUUGGCAAGCGGCAGGGCAGAGAGUUCAUCUGGAACGA |
| | | CCUGCUGUCUCUGGAGACCGGCAGCCUGAAGCUGGCCAACGGCAGAGUGAUCG |
| | | AGAAGACACUGUACAACAGACGAACCAGACAAGACGAGCCCGCCCUGUUUGUG |
| | | GCCCUGACCUUCGAGAGAAGAGAGGUGCUGGACAGCAGCAAUAUCAAGCCUAU |
| | | GAACCUGAUCGGCGUGGACCGGGGCGAGAACAUCCCUGCCGUGAUCGCCCUUA |
| | | CCGACCCCGAGGGAUGCCCUCUGAGCCGGUUUAAAGACAGCCUGGGCAACCCU |
| | | ACCCACAUCCUGAGAAUUGGCGAGUCCUACAAGGAGAAGCAGAGAACCAUCCA |
| | | GGCCAAGAAGGAGGUGGAGCAGCGGCGGGCUGGCGGCUACUCCCGGAAGUACG |
| | | CCAGCAAGGCCAAGAACCUGGCCGACGACAUGGUUAGAAAUACCGCCAGAGAC |
| | | CUCCUGUACUACGCUGUGACCCAGGACGCCAUGCUGAUCUUCGAGAACCUGAG |
| | | CAGAGGCUUCGGCAGACAGGGCAAGAGAACCUUCAUGGCCGAGAGACAGUACA |
| | | CCCGGAUGGAGGACUGGCUGACCGCCAAGCUGGCCUACGAGGGCCUGCCCUCU |
| | | AAGACCUACCUGUCCAAGACCUUGGCACAGUACACCAGCAAGACAUGCUCUAA |
| | | CUGCGGCUUCACAAUCACGAGCGCCGACUACGACCGGGUGCUGGAGAAACUGA |
| | | AGAAGACCGCCACAGGCUGGAUGACCACCAUUAACGGCAAGGAGCUGAAGGUG |
| | | GAGGGCCAGAUCACCUACUACAACAGGUACAAACGGCAGAACGUGGUGAAGGA |
| | | CCUGAGCGUGGAACUGGAUAGACUGAGCGAGGAAAGCGUAAACAAUGACAUCA |
| | | GCAGCUGGACCAAGGGCCGGAGCGGCGAGGCCCUGAGCCUGCUGAAGAAGAGA |
| | | UUCUCCCACAGACCAGUGCAGGAGAAGUUCGUGUGUCUGAACUGCGGCUUCGA |
| | | GACCCACGCCGACGAGCAAGCCGCCCUGAACAUCGCCCGGUCUUGGCUUUUCC |
| | | UGCGGAGCCAGGAGUACAAGAAGUACCAGACAAACAAGACCACAGGCAACACA |
| | | GACAAGAGAGCCUUCGUCGAGACCUGGCAGAGCUUCUACAGAAAGAAGCUGAA |
| | | GGAGGUGUGGAAGCCUGCCGUG |
| 19702 | 812 | CAGGAGAUCAAGCGGAUUAAUAAAAUUCGGAGAAGACUGGUGAAGGAUUCUAA |
| | | CACAAAGAAGGCUGGCAAGACAGGCCCUAUGAAGACACUGCUGGUGAGAGUGA |
| | | UGACACCCGACCUGAGAGAAAGACUGGAAAACCUGAGAAAGAAGCCUGAGAAU |
| | | AUCCCCCAGCCCAUCAGCAACACAAGCCGGGCCAACCUGAAUAAGCUGCUGAC |
| | | CGACUACACCGAAAUGAAGAAGGCCAUCCUGCACGUGUAUUGGGAAGAGUUCC |
| | | AGAAAGACCCAGUCGGCCUGAUGAGCAGAGUGGCUCAGCCUGCCAGCAAGAAG |
| | | AUCGAUCAGAACAAGCUGAAGCCCGAAAUGGACGAGAAGGGGAACCUGACAAC |
| | | CGCCGGCUUUGCCUGUAGCCAGUGCGGCCAGCCCCUGUUUGUGUACAAACUGG |
| | | AACAGGUGAGCGAAAAGGGCAAGGCUUACACGAAUUACUUCGGCAGAUGCAAC |
| | | GUGGCCGAGCACGAGAAGCUGAUCCUGCUGGCCCAGCUGAAGCCUGAGAAGGA |
| | | UAGCGAUGAGGCAGUGACAUAUUCCCUGGGCAAGUUCGGACAGCGGGCCCUGG |
| | | AUUUUUAUUCCAUUCAUGUGACCAAGGAAUCCACCCACCCCGUCAAGCCUCUU |
| | | GCCCAAAUUGCCGGCAACAGAUACGCCAGCGGCCCCGUGGGCAAGGCCCUGAG |
| | | CGACGCCUGUAUGGGCACCAUCGCCAGCUUCCUGUCUAAGUACCAGGACAUUA |
| | | UCAUCGAGCACCAGAAGGUGGUGAAGGGCAACCAGAAGAGACUGGAGAGCCUG |
| | | CGCGAGCUGGCCGGCAAGGAAAACCUGGAGUAUCCUAGCGUGACCCUGCCUCC |
| | | UCAGCCUCAUACAAAGGAGGGCGUGGAUGCCUACAACGAAGUGAUCGCCCGGG |
| | | UGCGGAUGUGGGUGAACCUGAAUCUGUGGCAGAAGCUGAAGCUGUCUAGAGAC |
| | | GACGCCAAGCCCCUGCUGAGACUGAAGAAGUUCCCCAGCUUCCCUCUGGUGGA |
| | | GAGACAGGCAAAUGAAGUGGACUGGUGGGACAUGGUGUGUAACGUGAAGAAGC |
| | | UGAUCAAUGAGAAGAAGGAGGACGGCAAAGUGUUCUGGCAGAAUCUGGCCGGC |
| | | UACAAGCGUCAGGAGGCCCUGCGGCCCUACCUGAGCAGCGAGGAAGACAGAAA |
| | | GAAGGGCAAGAAGUUCGCCCGGUAUCAGCUGGGGGACCUGCUGCUGCACCUCG |
| | | AGAAGAAGCACGGCGAAGACUGGGGGAAGGUGUACGAUGAGGCCUGGGAGCGG |
| | | AUCGAUAAGAAGGUGGAGGGCCUGAGCAAGCACAUCAAGCUGGAGGAGGAACG |
| | | GAGAUCUGAGGACGCCCAGAGCAAGGCCGCCCUGACCGACUGGCUGAGAGCCA |
| | | AGGCCAGCUUCGUCAUCGAGGGGCUGAAGGAGGCCGACAAGGACGAGUUCUGC |
| | | CGGUGCGAACUGAAGCUGCAGAAGUGGUACGGAGAUCUGAGAGGCAAACCUUU |
| | | CGCCAUCGAGGCCGAGAACAGCAUCCUGGACAUCAGCGGCUUCAGCAAGCAGU |
| | | ACAACUGCGCCUUUAUUUGGCAGAAGGACGGAGUGAAGAAGCUGAACCUGUAC |
| | | CUGAUCAUCAACUAUUUCAAGGGCGGCAAGCUGAGAUUCAAGAAGAUCAAGCC |
| | | UGAAGCCUUCGAGGCCAACAGAUUCUACACCGUGAUUAACAAGAAAAGCGGAG |
| | | AGAUCGUGCCAAUGGAAGUGAACUUCAACUUCGACGACCCUAACCUGAUCAUC |
| | | CUGCCCCUGGCAUUUGGCAAGCGGCAGGGCAGAGAGUUCAUCUGGAACGACCU |
| | | GCUGUCUCUGGAGACCGGCAGCCUGAAGCUGGCCAACGGCAGAGUGAUCGAGA |
| | | AGACACUGUACAACAGACGAACCAGACAAGACGAGCCCGCCCUGUUUGUGGCC |
| | | CUGACCUUCGAGAGAAGAGAGGUGCUGGACAGCAGCAAUAUCAAGCCUAUGAA |
| | | CCUGAUCGGCGUGGACCGGGGCGAGAACAUCCCUGCCGUGAUCGCCCUUACCG |
| | | ACCCCGAGGGAUGCCCUCUGAGCCGGUUUAAAGACAGCCUGGGCAACCCUACC |
| | | CACAUCCUGAGAAUUGGCGAGUCCUACAAGGAGAAGCAGAGAACCAUCCAGGC |
| | | CAAGAAGGAGGUGGAGCAGCGGCGGGCUGGCGGCUACUCCCGGAAGUACGCCA |
| | | GCAAGGCCAAGAACCUGGCCGACGACAUGGUUAGAAAUACCGCCAGAGACCUC |
| | | CUGUACUACGCUGUGACCCAGGACGCCAUGCUGAUCUUCGAGAACCUGAGCAG |
| | | AGGCUUCGGCAGACAGGGCAAGAGAACCUUCAUGGCCGAGAGACAGUACACCC |
| | | GGAUGGAGGACUGGCUGACCGCCAAGCUGGCCUACGAGGGCCUGCCCUCUAAG |
| | | ACCUACCUGUCCAAGACCUUGGCACAGUACACCAGCAAGACAUGCUCUAACUG |
| | | CGGCUUCACAAUCACGAGCGCCGACUACGACCGGGUGCUGGAGAAACUGAAGA |
| | | AGACCGCCACAGGCUGGAUGACCACCAUUAACGGCAAGGAGCUGAAGGUGGAG |
| | | GGCCAGAUCACCUACUACAACAGGUACAAACGGCAGAACGUGGUGAAGGACCU |
| | | GAGCGUGGAACUGGAUAGACUGAGCGAGGAAAGCGUAAACAAUGACAUCAGCA |
| | | GCUGGACCAAGGGCCGGAGCGGCGAGGCCCUGAGCCUGCUGAAGAAGAGAUUC |
| | | UCCCACAGACCAGUGCAGGAGAAGUUCGUGUGUCUGAACUGCGGCUUCGAGAC |
| | | CCACGCCGACGAGCAAGCCGCCCUGAACAUCGCCCGGUCUUGGCUUUUCCUGC |
| | | GGAGCCAGGAGUACAAGAAGUACCAGACAAACAAGACCACAGGCAACACAGAC |
| | | AAGAGAGCCUUCGUCGAGACCUGGCAGAGCUUCUACAGAAAGAAGCUGAAGGA |
| | | GGUGUGGAAGCCUGCCGUG |
| 19817 | N1- | CAGGAGAmΨCAAGCGGAmΨmΨAAmΨAAAAmΨmΨCGGAGAAGACmΨGGmΨGAAG |
| | methylpseudo | GAmΨmΨCmΨAACACAAAGAAGGCmΨGGCAAGACAGGCCCmΨAmΨGAAGACACm |
| | uridine | ΨGCmΨGGmΨGAGAGmΨGAmΨGACACCCGACCmΨGAGAGAAAGACmΨGGAAAAC |
| | substituted | CmΨGAGAAAGAAGCCmΨGAGAAmΨAmΨCCCCCAGCCCAmΨCAGCAACACAAGC |
| | RNA sequence | CGGGCCAACCmΨGAAmΨAAGCmΨGCmΨGACCGACmΨACACCGAAAmΨGAAGAA |
| | encoding | GGCCAmΨCCmΨGCACGmΨGmΨAmΨmΨGGGAAGAGmΨmΨCCAGAAAGACCCAGm |
| | CasX 491 | ΨCGGCCmΨGAmΨGAGCAGAGmΨGGCmΨCAGCCmΨGCCAGCAAGAAGAmΨCGAm |
| | | ΨCAGAACAAGCmΨGAAGCCCGAAAmΨGGACGAGAAGGGGAACCmΨGACAACCG |
| | | CCGGCmΨmΨmΨGCCmΨGmΨAGCCAGmΨGCGGCCAGCCCCmΨGmΨmΨmΨGmΨGm |
| | | ΨACAAACmΨGGAACAGGmΨGAGCGAAAAGGGCAAGGCmΨmΨACACGAAmΨmΨA |
| | | CmΨmΨCGGCAGAmΨGCAACGmΨGGCCGAGCACGAGAAGCmΨGAmΨCCmΨGCmΨ |
| | | GGCCCAGCmΨGAAGCCmΨGAGAAGGAmTAGCGAmΨGAGGCAGmΨGACAmΨAmΨ |
| | | mΨCCCmΨGGGCAAGmΨmΨCGGACAGCGGGCCCmΨGGAmΨmΨmΨmΨmΨAmΨmΨC |
| | | CAmΨmΨCAmΨGmΨGACCAAGGAAmΨCCACCCACCCCGmΨCAAGCCmΨCmΨmΨG |
| | | CCCAAAmΨmΨGCCGGCAACAGAmΨACGCCAGCGGCCCCGmΨGGGCAAGGCCCm |
| | | ΨGAGCGACGCCmΨGmΨAmΨGGGCACCAmΨCGCCAGCmΨmΨCCmΨGmΨCmΨAAG |
| | | mΨACCAGGACAmΨmΨAmΨCAmΨCGAGCACCAGAAGGmΨGGmΨGAAGGGCAACC |
| | | AGAAGAGACmΨGGAGAGCCmΨGCGCGAGCmΨGGCCGGCAAGGAAAACCmΨGGA |
| | | GmΨAmΨCCmΨAGCGmΨGACCCmΨGCCmΨCCmΨCAGCCmΨCAmΨACAAAGGAGG |
| | | GCGmΨGGAmΨGCCmΨACAACGAAGmΨGAmΨCGCCCGGGmΨGCGGAmΨGmΨGGG |
| | | mΨGAACCmΨGAAmΨCmΨGmΨGGCAGAAGCmΨGAAGCmΨGmΨCmΨAGAGACGAC |
| | | GCCAAGCCCCmΨGCmΨGAGACmΨGAAGGGCmΨmΨCCCCAGCmΨmΨCCCmΨCmΨ |
| | | GGmΨGGAGAGACAGGCAAAmΨGAAGmΨGGACmΨGGmΨGGGACAmΨGGmΨGmΨG |
| | | mΨAACGmΨGAAGAAGCmΨGAmΨCAAmΨGAGAAGAAGGAGGACGGCAAAGmΨGm |
| | | ΨmΨCmΨGGCAGAAmΨCmΨGGCCGGCmΨACAAGCGmΨCAGGAGGCCCmΨGCGGC |
| | | CCmΨACCmΨGAGCAGCGAGGAAGACAGAAAGAAGGGCAAGAAGmΨmΨCGCCCG |
| | | GmΨAmΨCAGCmΨGGGGGACCmΨGCmΨGCmΨGCACCmΨCGAGAAGAAGCACGGC |
| | | GAAGACmΨGGGGGAAGGmΨGmΨACGAmΨGAGGCCmΨGGGAGCGGAmΨCGAmΨA |
| | | AGAAGGmΨGGAGGGCCmΨGAGCAAGCACAmΨCAAGCmΨGGAGGAGGAACGGAG |
| | | AmΨCmΨGAGGACGCCCAGAGCAAGGCCGCCCmΨGACCGACmΨGGCmΨGAGAGC |
| | | CAAGGCCAGCmΨmΨCGmΨCAmΨCGAGGGGCmΨGAAGGAGGCCGACAAGGACGA |
| | | GmΨmΨCmΨGCCGGmΨGCGAACmΨGAAGCmΨGCAGAAGmΨGGmΨACGGAGAmΨC |
| | | mΨGAGAGGCAAACCmΨmΨmΨCGCCAmΨCGAGGCCGAGAACAGCAmΨCCmΨGGA |
| | | CAmΨCAGCGGCmΨmΨCAGCAAGCAGmΨACAACmΨGCGCCmΨmΨmΨAmΨmΨmΨG |
| | | GCAGAAGGACGGAGmΨGAAGAAGCmΨGAACCmΨGmΨACCmΨGAmΨCAmΨCAAC |
| | | mΨAmΨmΨmΨCAAGGGCGGCAAGCmΨGAGAmΨmΨCAAGAAGAmΨCAAGCCmΨGA |
| | | AGCCmΨmΨCGAGGCCAACAGAmΨmΨCmΨACACCGmΨGAmΨmΨAACAAGAAAAG |
| | | CGGAGAGAmΨCGmΨGCCAAmΨGGAAGmΨGAACmΨmΨCAACmΨmΨCGACGACCC |
| | | mΨAACCmΨGAmΨCAmΨCCmΨGCCCCmΨGGCAmΨmΨmΨGGCAAGCGGCAGGGCA |
| | | GAGAGmΨmΨCAmΨCmΨGGAACGACCmΨGCmΨGmΨCmΨCmΨGGAGACCGGCAGC |
| | | CmΨGAAGCmΨGGCCAACGGCAGAGmΨGAmΨCGAGAAGACACmΨGmΨACAACAG |
| | | ACGAACCAGACAAGACGAGCCCGCCCmΨGmΨmΨmΨGmΨGGCCCmΨGACCmΨmΨ |
| | | CGAGAGAAGAGAGGmΨGCmΨGGACAGCAGCAAmΨAmΨCAAGCCmΨAmΨGAACC |
| | | mΨGAmΨCGGCGmΨGGACCGGGGCGAGAACAmΨCCCmΨGCCGmΨGAmΨCGCCCm |
| | | ΨmΨACCGACCCCGAGGGAmΨGCCCmΨCmΨGAGCCGGmΨmΨmΨAAAGACAGCCm |
| | | ΨGGGCAACCCmΨACCCACAmΨCCmΨGAGAAmΨmΨGGCGAGmΨCCmΨACAAGGA |
| | | GAAGCAGAGAACCAmΨCCAGGCCAAGAAGGAGGmΨGGAGCAGCGGCGGGCmΨG |
| | | GCGGCmΨACmΨCCCGGAAGmΨACGCCAGCAAGGCCAAGAACCmΨGGCCGACGA |
| | | CAmΨGGmΨmΨAGAAAmΨACCGCCAGAGACCmΨCCmΨGmΨACmΨACGCmΨGmΨG |
| | | ACCCAGGACGCCAmΨGCmΨGAmΨCmΨmΨCGAGAACCmΨGAGCAGAGGCmΨmΨC |
| | | GGCAGACAGGGCAAGAGAACCmΨmΨCAmΨGGCCGAGAGACAGmΨACACCCGGA |
| | | mΨGGAGGACmΨGGCmΨGACCGCCAAGCmΨGGCCmΨACGAGGGCCmΨGmΨCmΨA |
| | | AGACCmΨACCmΨGmΨCCAAGACCmΨmΨGGCACAGmΨACACCAGCAAGACAmΨG |
| | | CmΨCmΨAACmΨGCGGCmΨmΨCACAAmΨCACGAGCGCCGACmΨACGACCGGGmΨ |
| | | GCmΨGGAGAAACmΨGAAGAAGACCGCCACAGGCmΨGGAmΨGACCACCAmΨmΨA |
| | | ACGGCAAGGAGCmΨGAAGGmΨGGAGGGCCAGAmΨCACCmΨACmΨACAACAGGm |
| | | ΨACAAACGGCAGAACGmΨGGmΨGAAGGACCmΨGAGCGmΨGGAACmΨGGAmTAG |
| | | ACmΨGAGCGAGGAAAGCGmΨAAACAAmΨGACAmΨCAGCAGCmΨGGACCAAGGG |
| | | CCGGAGCGGCGAGGCCCmΨGAGCCmΨGCmΨGAAGAAGAGAmΨmΨCmΨCCCACA |
| | | GACCAGmΨGCAGGAGAAGmΨmΨCGmΨGmΨGmΨCmΨGAACmΨGCGGCmΨmΨCGA |
| | | GACCCACGCCGACGAGCAAGCCGCCCmΨGAACAmΨCGCCCGGmΨCmΨmΨGGCm |
| | | ΨmΨmΨmΨCCmΨGCGGAGCCAGGAGmΨACAAGAAGmΨACCAGACAAACAAGACC |
| | | ACAGGCAACACAGACAAGAGAGCCmΨmΨCGmΨCGAGACCmΨGGCAGAGCmΨmΨ |
| | | CmΨACAGAAAGAAGCmΨGAAGGAGGmΨGmΨGGAAGCCmΨGCCGmΨG |
| 19818 | N1- | CAGGAGAmΨCAAGCGGAmΨmΨAAmΨAAAAmΨmΨCGGAGAAGACmΨGGmΨGAAG |
| | methylpseudo | GAmΨmΨCmΨAACACAAAGAAGGCmΨGGCAAGACAGGCCCmΨAmΨGAAGACACm |
| | uridine | ΨGCmΨGGmΨGAGAGmΨGAmΨGACACCCGACCmΨGAGAGAAAGACmΨGGAAAAC |
| | substituted | CmΨGAGAAAGAAGCCmΨGAGAAmΨAmΨCCCCCAGCCCAmΨCAGCAACACAAGC |
| | RNA sequence | CGGGCCAACCmΨGAAmΨAAGCmΨGCmΨGACCGACmΨACACCGAAAmΨGAAGAA |
| | encoding | GGCCAmΨCCmΨGCACGmΨGmΨAmΨmΨGGGAAGAGmΨmΨCCAGAAAGACCCAGm |
| | CasX 515 | ΨCGGCCmΨGAmΨGAGCAGAGmΨGGCmΨCAGCCmΨGCCAGCAAGAAGAmΨCGAm |
| | | ΨCAGAACAAGCmΨGAAGCCCGAAAmΨGGACGAGAAGGGGAACCmΨGACAACCG |
| | | CCGGCmΨmΨmΨGCCmΨGmΨAGCCAGmΨGCGGCCAGCCCCmΨGmΨmΨmΨGmΨGm |
| | | ΨACAAACmΨGGAACAGGmΨGAGCGAAAAGGGCAAGGCmΨmΨACACGAAmΨmΨA |
| | | CmΨmΨCGGCAGAmΨGCAACGmΨGGCCGAGCACGAGAAGCmΨGAmΨCCmΨGCmΨ |
| | | GGCCCAGCmΨGAAGCCmΨGAGAAGGAmΨAGCGAmΨGAGGCAGmΨGACAmΨAmΨ |
| | | mΨCCCmΨGGGCAAGmΨmΨCGGACAGCGGGCCCmΨGGAmΨmΨmΨmΨmΨAmΨmΨC |
| | | CAmΨmΨCAmΨGmΨGACCAAGGAAmΨCCACCCACCCCGmΨCAAGCCmΨCmΨmΨG |
| | | CCCAAAmΨmΨGCCGGCAACAGAmΨACGCCAGCGGCCCCGmΨGGGCAAGGCCCm |
| | | ΨGAGCGACGCCmΨGmΨAmΨGGGCACCAmΨCGCCAGCmΨmΨCCmΨGmΨCmΨAAG |
| | | mΨACCAGGACAmΨmΨAmΨCAmΨCGAGCACCAGAAGGmΨGGmΨGAAGGGCAACC |
| | | AGAAGAGACmΨGGAGAGCCmΨGCGCGAGCmΨGGCCGGCAAGGAAAACCmΨGGA |
| | | GmΨAmΨCCmΨAGCGmΨGACCCmΨGCCmΨCCmΨCAGCCmΨCAmΨACAAAGGAGG |
| | | GCGmΨGGAmΨGCCmΨACAACGAAGmΨGAmΨCGCCCGGGmΨGCGGAmΨGmΨGGG |
| | | mΨGAACCmΨGAAmΨCmΨGmΨGGCAGAAGCmΨGAAGCmΨGmΨCmΨAGAGACGAC |
| | | GCCAAGCCCCmΨGCmΨGAGACmΨGAAGGGCmΨmΨCCCCAGCmΨmΨCCCmΨCmΨ |
| | | GGmΨGGAGAGACAGGCAAAmΨGAAGmΨGGACmΨGGmΨGGGACAmΨGGmΨGmΨG |
| | | mΨAACGmΨGAAGAAGCmΨGAmΨCAAmΨGAGAAGAAGGAGGACGGCAAAGmΨGm |
| | | ΨmΨCmΨGGCAGAAmΨCmΨGGCCGGCmΨACAAGCGmΨCAGGAGGCCCmΨGCGGC |
| | | CCmΨACCmΨGAGCAGCGAGGAAGACAGAAAGAAGGGCAAGAAGmΨmΨCGCCCG |
| | | GmΨAmΨCAGCmΨGGGGGACCmΨGCmΨGCmΨGCACCmΨCGAGAAGAAGCACGGC |
| | | GAAGACmΨGGGGGAAGGmΨGmΨACGAmΨGAGGCCmΨGGGAGCGGAmΨCGAmΨA |
| | | AGAAGGmΨGGAGGGCCmΨGAGCAAGCACAmΨCAAGCmΨGGAGGAGGAACGGAG |
| | | AmΨCmΨGAGGACGCCCAGAGCAAGGCCGCCCmΨGACCGACmΨGGCmΨGAGAGC |
| | | CAAGGCCAGCmΨmΨCGmΨCAmΨCGAGGGGCmΨGAAGGAGGCCGACAAGGACGA |
| | | GmΨmΨCmΨGCCGGmΨGCGAACmΨGAAGCmΨGCAGAAGmΨGGmΨACGGAGAmΨC |
| | | mΨGAGAGGCAAACCmΨmΨmΨCGCCAmΨCGAGGCCGAGAACAGCAmΨCCmΨGGA |
| | | CAmΨCAGCGGCmΨmΨCAGCAAGCAGmΨACAACmΨGCGCCmΨmΨmΨAmΨmΨmΨG |
| | | GCAGAAGGACGGAGmΨGAAGAAGCmΨGAACCmΨGmΨACCmΨGAmΨCAmΨCAAC |
| | | mΨAmΨmΨmΨCAAGGGCGGCAAGCmΨGAGAmΨmΨCAAGAAGAmΨCAAGCCmΨGA |
| | | AGCCmΨmΨCGAGGCCAACAGAmΨmΨCmΨACACCGmΨGAmΨmΨAACAAGAAAAG |
| | | CGGAGAGAmΨCGmΨGCCAAmΨGGAAGmΨGAACmΨmΨCAACmΨmΨCGACGACCC |
| | | mΨAACCmΨGAmΨCAmΨCCmΨGCCCCmΨGGCAmΨmΨmΨGGCAAGCGGCAGGGCA |
| | | GAGAGmΨmΨCAmΨCmΨGGAACGACCmΨGCmΨGmΨCmΨCmΨGGAGACCGGCAGC |
| | | CmΨGAAGCmΨGGCCAACGGCAGAGmΨGAmΨCGAGAAGACACmΨGmΨACAACAG |
| | | ACGAACCAGACAAGACGAGCCCGCCCmΨGmΨmΨmΨGmΨGGCCCmΨGACCmΨmΨ |
| | | CGAGAGAAGAGAGGmΨGCmΨGGACAGCAGCAAmΨAmΨCAAGCCmΨAmΨGAACC |
| | | mΨGAmΨCGGCGmΨGGACCGGGGCGAGAACAmΨCCCmΨGCCGmΨGAmΨCGCCCm |
| | | ΨmΨACCGACCCCGAGGGAmΨGCCCmΨCmΨGAGCCGGmΨmΨmΨAAAGACAGCCm |
| | | ΨGGGCAACCCmΨACCCACAmΨCCmΨGAGAAmΨmΨGGCGAGmΨCCmΨACAAGGA |
| | | GAAGCAGAGAACCAmΨCCAGGCCAAGAAGGAGGmΨGGAGCAGCGGCGGGCmΨG |
| | | GCGGCmΨACmΨCCCGGAAGmΨACGCCAGCAAGGCCAAGAACCmΨGGCCGACGA |
| | | CAmΨGGmΨmΨAGAAAmΨACCGCCAGAGACCmΨCCmΨGmΨACmΨACGCmΨGmΨG |
| | | ACCCAGGACGCCAmΨGCmΨGAmΨCmΨmΨCGAGAACCmΨGAGCAGAGGCmΨmΨC |
| | | GGCAGACAGGGCAAGAGAACCmΨmΨCAmΨGGCCGAGAGACAGmΨACACCCGGA |
| | | mΨGGAGGACmΨGGCmΨGACCGCCAAGCmΨGGCCmΨACGAGGGCCmΨGCCCmΨC |
| | | mΨAAGACCmΨACCmΨGmΨCCAAGACCmΨmΨGGCACAGmΨACACCAGCAAGACA |
| | | mΨGCmΨCmΨAACmΨGCGGCmΨmΨCACAAmΨCACGAGCGCCGACmΨACGACCGG |
| | | GmΨGCmΨGGAGAAACmΨGAAGAAGACCGCCACAGGCmΨGGAmΨGACCACCAmΨ |
| | | mΨAACGGCAAGGAGCmΨGAAGGmΨGGAGGGCCAGAmΨCACCmΨACmΨACAACA |
| | | GGmΨACAAACGGCAGAACGmΨGGmΨGAAGGACCmΨGAGCGmΨGGAACmΨGGAm |
| | | ΨAGACmΨGAGCGAGGAAAGCGmΨAAACAAmΨGACAmΨCAGCAGCmΨGGACCAA |
| | | GGGCCGGAGCGGCGAGGCCCmΨGAGCCmΨGCmΨGAAGAAGAGAmΨmΨCmΨCCC |
| | | ACAGACCAGmΨGCAGGAGAAGmΨmΨCGmΨGmΨGmΨCmΨGAACmΨGCGGCmΨmΨ |
| | | CGAGACCCACGCCGACGAGCAAGCCGCCCmΨGAACAmΨCGCCCGGmΨCmΨmΨG |
| | | GCmΨmΨmΨmΨCCmΨGCGGAGCCAGGAGmΨACAAGAAGmΨACCAGACAAACAAG |
| | | ACCACAGGCAACACAGACAAGAGAGCCmΨmΨCGmΨCGAGACCmΨGGCAGAGCm |
| | | ΨmΨCmΨACAGAAAGAAGCmΨGAAGGAGGmΨGmΨGGAAGCCmΨGCCGmΨG |
| 19819 | N1- | CAGGAGAmΨCAAGCGGAmΨmΨAAmΨAAAAmΨmΨCGGAGAAGACmΨGGmΨGAAG |
| | methylpseudo | GAmΨmΨCmΨAACACAAAGAAGGCmΨGGCAAGACACGGGGCCCmΨAmΨGAAGAC |
| | uridine | ACmΨGCmΨGGmΨGAGAGmΨGAmΨGACACCCGACCmΨGAGAGAAAGACmΨGGAA |
| | substituted | AACCmΨGAGAAAGAAGCCmΨGAGAAmΨAmΨCCCCCAGCCCAmΨCAGCAACACA |
| | RNA | AGCCGGGCCAACCmΨGAAmΨAAGCmΨGCmΨGACCGACmΨACACCGAAAmΨGAA |
| | sequence | GAAGGCCAmΨCCmΨGCACGmΨGmΨAmΨmΨGGGAAGAGmΨmΨCCAGAAAGACCC |
| | encoding | AGmΨCGGCCmΨGAmΨGAGCAGAGmΨGGCmΨCAGCCmΨGCCAGCAAGAAGAmΨC |
| | CasX 676 | GAmΨCAGAACAAGCmΨGAAGCCCGAAAmΨGGACGAGAAGGGGAACCmΨGACAA |
| | | CCGCCGGCmΨmΨmΨGCCmΨGmΨAGCCAGmΨGCGGCCAGCCCCmΨGmΨmΨmΨGm |
| | | ΨGmΨACAAACmΨGGAACAGGmΨGAGCGAAAAGGGCAAGGCmΨmΨACACGAAmΨ |
| | | mΨACmΨmΨCGGCAGAmΨGCAACGmΨGGCCGAGCACGAGAAGCmΨGAmΨCAAGC |
| | | mΨGGCCCAGCmΨGAAGCCmΨGAGAAGGAmTAGCGAmΨGAGGCAGmΨGACAmΨA |
| | | mΨmΨCCCmΨGGGCAAGmΨmΨCGGACAGCGGGCCCmΨGGAmΨmΨmΨmΨmΨAmΨm |
| | | ΨCCAmΨmΨCAmΨGmΨGACCAAGGAAmΨCCACCCACCCCGmΨCAAGCCmΨCmΨm |
| | | ΨGCCCAAAmΨmΨGCCGGCAACAGAmΨACGCCmΨCCAGCCCCGmΨGGGCAAGGC |
| | | CCmΨGAGCGACGCCmΨGmΨAmΨGGGCACCAmΨCGCCAGCmΨmΨCCmΨGmΨCmΨ |
| | | AAGmΨACCAGGACAmΨmΨAmΨCAmΨCGAGCACCAGAAGGmΨGGmΨGAAGGGCA |
| | | ACCAGAAGAGACmΨGGAGAGCCmΨGCGCGAGCmΨGGCCGGCAAGGAAAACCmΨ |
| | | GGAGmΨAmΨCCmΨAGCGmΨGACCCmΨGCCmΨCCmΨCAGCCmΨCAmΨACAAAGG |
| | | AGGGCGmΨGGAmΨGCCmΨACAACGAAGmΨGAmΨCGCCCGGGmΨGCGGAmΨGmΨ |
| | | GGGmΨGAACCmΨGAAmΨCmΨGmΨGGCAGAAGCmΨGAAGCmΨGmΨCmΨAGAGAC |
| | | GACGCCAAGCCCCmΨGCmΨGAGACmΨGAAGGGCmΨmΨCCCCAGCmΨmΨCCCmΨ |
| | | CmΨGGmΨGGAGAGACAGGCAAAmΨGAAGmΨGGACmΨGGmΨGGGACAmΨGGmΨG |
| | | mΨGmΨAACGmΨGAAGAAGCmΨGAmΨCAAmΨGAGAAGAAGGAGGACGGCAAAGm |
| | | ΨGmΨmΨCmΨGGCAGAAmΨCmΨGGCCGGCmΨACAAGCGmΨCAGGAGGCCCmΨGC |
| | | GGCCCmΨACCmΨGAGCAGCGAGGAAGACAGAAAGAAGGGCAAGAAGmΨmΨCGC |
| | | CCGGmΨAmΨCAGCmΨGGGGGACCmΨGCmΨGCmΨGCACCmΨCGAGAAGAAGCAC |
| | | GGCGAAGACmΨGGGGGAAGGmΨGmΨACGAmΨGAGGCCmΨGGGAGCGGAmΨCGA |
| | | mΨAAGAAGGmΨGGAGGGCCmΨGAGCAAGCACAmΨCAAGCmΨGGAGGAGGAACG |
| | | GAGAmΨCmΨGAGGACGCCCAGAGCAAGGCCGCCCmΨGACCGACmΨGGCmΨGAG |
| | | AGCCAAGGCCAGCmΨmΨCGmΨCAmΨCGAGGGGCmΨGAAGGAGGCCGACAAGGA |
| | | CGAGmΨmΨCmΨGCCGGmΨGCGAACmΨGAAGCmΨGCAGAAGmΨGGmΨACGGAGA |
| | | mΨCmΨGAGAGGCAAACCmΨmΨmΨCGCCAmΨCGAGGCCGAGAACAGCAmΨCCmΨ |
| | | GGACAmΨCAGCGGCmΨmΨCAGCAAGCAGmΨACAACmΨGCGCCmΨmΨmΨAmΨmΨ |
| | | mΨGGCAGAAGGACGGAGmΨGAAGAAGCmΨGAACCmΨGmΨACCmΨGAmΨCAmΨC |
| | | AACmΨAmΨmΨmΨCAAGGGCGGCAAGCmΨGAGAmΨmΨCAAGAAGAmΨCAAGCCm |
| | | ΨGAAGCCmΨmΨCGAGGCCAACAGAmΨmΨCmΨACACCGmΨGAmΨmΨAACAAGAA |
| | | AAGCGGAGAGAmΨCGmΨGCCAAmΨGGAAGmΨGAACmΨmΨCAACmΨmΨCGACGA |
| | | CCCmΨAACCmΨGAmΨCAmΨCCmΨGCCCCmΨGGCAmΨmΨmΨGGCAAGCGGCAGG |
| | | GCAGAGAGmΨmΨCAmΨCmΨGGAACGACCmΨGCmΨGmΨCmΨCmΨGGAGACCGGC |
| | | AGCCmΨGAAGCmΨGGCCAACGGCAGAGmΨGAmΨCGAGAAGACACmΨGmΨACAA |
| | | CAGACGAACCAGACAAGACGAGCCCGCCCmΨGmΨmΨmΨGmΨGGCCCmΨGACCm |
| | | ΨmΨCGAGAGAAGAGAGGmΨGCmΨGGACAGCAGCAAmΨAmΨCAAGCCmΨAmΨGA |
| | | ACCmΨGAmΨCGGCGmΨGGACCGGGGCGAGAACAmΨCCCmΨGCCGmΨGAmΨCGC |
| | | CCmΨmΨACCGACCCCGAGGGAmΨGCCCmΨCmΨGAGCCGGmΨmΨmΨAAAGACAG |
| | | CCmΨGGGCAACCCmΨACCCACAmΨCCmΨGAGAAmΨmΨGGCGAGmΨCCmΨACAA |
| | | GGAGAAGCAGAGAACCAmΨCCAGGCCAAGAAGGAGGmΨGGAGCAGCGGCGGGC |
| | | mΨGGCGGCmΨACmΨCCCGGAAGmΨACGCCAGCAAGGCCAAGAACCmΨGGCCGA |
| | | CGACAmΨGGmΨmΨAGAAAmΨACCGCCAGAGACCmΨCCmΨGmΨACmΨACGCmΨG |
| | | mΨGACCCAGGACGCCAmΨGCmΨGAmΨCmΨmΨCGAGAACCmΨGAGCAGAGGCmΨ |
| | | mΨCGGCAGACAGGGCAAGAGAACCmΨmΨCAmΨGGCCGAGAGACAGmΨACACCC |
| | | GGAmΨGGAGGACmΨGGCmΨGACCGCCAAGCmΨGGCCmΨACGAGGGCCmΨGCCC |
| | | mΨCmΨAAGACCmΨACCmΨGmΨCCAAGACCmΨmΨGGCACAGmΨACACCAGCAAG |
| | | ACAmΨGCmΨCmΨAACmΨGCGGCmΨmΨCACAAmΨCACGAGCGCCGACmΨACGAC |
| | | CGGGmΨGCmΨGGAGAAACmΨGAAGAAGACCGCCACAGGCmΨGGAmΨGACCACC |
| | | AmΨmΨAACGGCAAGGAGCmΨGAAGGmΨGGAGGGCCAGAmΨCACCmΨACmΨACA |
| | | ACAGGmΨACAAACGGCAGAACGmΨGGmΨGAAGGACCmΨGAGCGmΨGGAACmΨG |
| | | GAmΨAGACmΨGAGCGAGGAAAGCGmΨAAACAAmΨGACAmΨCAGCAGCmΨGGAC |
| | | CAAGGGCCGGAGCGGCGAGGCCCmΨGAGCCmΨGCmΨGAAGAAGAGAmΨmΨCmΨ |
| | | CCCACAGACCAGmΨGCAGGAGAAGmΨmΨCGmΨGmΨGmΨCmΨGAACmΨGCGGCm |
| | | ΨmΨCGAGACCCACGCCGACGAGCAAGCCGCCCmΨGAACAmΨCGCCCGGmΨCmΨ |
| | | mΨGGCmΨmΨmΨmΨCCmΨGCGGAGCCAGGAGmΨACAAGAAGmΨACCAGACAAAC |
| | | AAGACCACAGGCAACACAGACAAGAGAGCCmΨmΨCGmΨCGAGACCmΨGGCAGA |
| | | GCmΨmΨCmΨACAGAAAGAAGCmΨGAAGGAGGmΨGmΨGGAAGCCmΨGCCGmΨG |
| 19820 | N1- | CAGGAGAmΨCAAGCGGAmΨmΨAAmΨAAAAmΨmΨCGGAGAAGACmΨGGmΨGAAG |
| | methylpseudo | GAmΨmΨCmΨAACACAAAGAAGGCmΨGGCAAGACAGGCCCmΨAmΨGAAGACACm |
| | uridine | ΨGCmΨGGmΨGAGAGmΨGAmΨGACACCCGACCmΨGAGAGAAAGACmΨGGAAAAC |
| | substituted | CmΨGAGAAAGAAGCCmΨGAGAAmΨAmΨCCCCCAGCCCAmΨCAGCAACACAAGC |
| | RNA | CGGGCCAACCmΨGAAmΨAAGCmΨGCmΨGACCGACmΨACACCGAAAmΨGAAGAA |
| | sequence | GGCCAmΨCCmΨGCACGmΨGmΨAmΨmΨGGGAAGAGmΨmΨCCAGAAAGACCCAGm |
| | encoding | ΨCGGCCmΨGAmΨGAGCAGAGmΨGGCmΨCAGCCmΨGCCAGCAAGAAGAmΨCGAm |
| | CasX 812 | ΨCAGAACAAGCmΨGAAGCCCGAAAmΨGGACGAGAAGGGGAACCmΨGACAACCG |
| | | CCGGCmΨmΨmΨGCCmΨGmΨAGCCAGmΨGCGGCCAGCCCCmΨGmΨmΨmΨGmΨGm |
| | | ΨACAAACmΨGGAACAGGmΨGAGCGAAAAGGGCAAGGCmΨmΨACACGAAmΨmΨA |
| | | CmΨmΨCGGCAGAmΨGCAACGmΨGGCCGAGCACGAGAAGCmΨGAmΨCCmΨGCmΨ |
| | | GGCCCAGCmΨGAAGCCmΨGAGAAGGAmΨAGCGAmΨGAGGCAGmΨGACAmΨAmΨ |
| | | mΨCCCmΨGGGCAAGmΨmΨCGGACAGCGGGCCCmΨGGAmΨmΨmΨmΨmΨAmΨmΨC |
| | | CAmΨmΨCAmΨGmΨGACCAAGGAAmΨCCACCCACCCCGmΨCAAGCCmΨCmΨmΨG |
| | | CCCAAAmΨmΨGCCGGCAACAGAmΨACGCCAGCGGCCCCGmΨGGGCAAGGCCCm |
| | | ΨGAGCGACGCCmΨGmΨAmΨGGGCACCAmΨCGCCAGCmΨmΨCCmΨGmΨCmΨAAG |
| | | mΨACCAGGACAmΨmΨAmΨCAmΨCGAGCACCAGAAGGmΨGGmΨGAAGGGCAACC |
| | | AGAAGAGACmΨGGAGAGCCmΨGCGCGAGCmΨGGCCGGCAAGGAAAACCmΨGGA |
| | | GmΨAmΨCCmΨAGCGmΨGACCCmΨGCCmΨCCmΨCAGCCmΨCAmΨACAAAGGAGG |
| | | GCGmΨGGAmΨGCCmΨACAACGAAGmΨGAmΨCGCCCGGGmΨGCGGAmΨGmΨGGG |
| | | mΨGAACCmΨGAAmΨCmΨGmΨGGCAGAAGCmΨGAAGCmΨGmΨCmΨAGAGACGAC |
| | | GCCAAGCCCCmΨGCmΨGAGACmΨGAAGAAGmΨmΨCCCCAGCmΨmΨCCCmΨCmΨ |
| | | GGmΨGGAGAGACAGGCAAAmΨGAAGmΨGGACmΨGGmΨGGGACAmΨGGmΨGmΨG |
| | | mΨAACGmΨGAAGAAGCmΨGAmΨCAAmΨGAGAAGAAGGAGGACGGCAAAGmΨGm |
| | | ΨmΨCmΨGGCAGAAmΨCmΨGGCCGGCmΨACAAGCGmΨCAGGAGGCCCmΨGCGGC |
| | | CCmΨACCmΨGAGCAGCGAGGAAGACAGAAAGAAGGGCAAGAAGmΨmΨCGCCCG |
| | | GmΨAmΨCAGCmΨGGGGGACCmΨGCmΨGCmΨGCACCmΨCGAGAAGAAGCACGGC |
| | | GAAGACmΨGGGGGAAGGmΨGmΨACGAmΨGAGGCCmΨGGGAGCGGAmΨCGAmΨA |
| | | AGAAGGmΨGGAGGGCCmΨGAGCAAGCACAmΨCAAGCmΨGGAGGAGGAACGGAG |
| | | AmΨCmΨGAGGACGCCCAGAGCAAGGCCGCCCmΨGACCGACmΨGGCmΨGAGAGC |
| | | CAAGGCCAGCmΨmΨCGmΨCAmΨCGAGGGGCmΨGAAGGAGGCCGACAAGGACGA |
| | | GmΨmΨCmΨGCCGGmΨGCGAACmΨGAAGCmΨGCAGAAGmΨGGmΨACGGAGAmΨC |
| | | mΨGAGAGGCAAACCmΨmΨmΨCGCCAmΨCGAGGCCGAGAACAGCAmΨCCmΨGGA |
| | | CAmΨCAGCGGCmΨmΨCAGCAAGCAGmΨACAACmΨGCGCCmΨmΨmΨAmΨmΨmΨG |
| | | GCAGAAGGACGGAGmΨGAAGAAGCmΨGAACCmΨGmΨACCmΨGAmΨCAmΨCAAC |
| | | mΨAmΨmΨmΨCAAGGGCGGCAAGCmΨGAGAmΨmΨCAAGAAGAmΨCAAGCCmΨGA |
| | | AGCCmΨmΨCGAGGCCAACAGAmΨmΨCmΨACACCGmΨGAmΨmΨAACAAGAAAAG |
| | | CGGAGAGAmΨCGmΨGCCAAmΨGGAAGmΨGAACmΨmΨCAACmΨmΨCGACGACCC |
| | | mΨAACCmΨGAmΨCAmΨCCmΨGCCCCmΨGGCAmΨmΨmΨGGCAAGCGGCAGGGCA |
| | | GAGAGmΨmΨCAmΨCmΨGGAACGACCmΨGCmΨGmΨCmΨCmΨGGAGACCGGCAGC |
| | | CmΨGAAGCmΨGGCCAACGGCAGAGmΨGAmΨCGAGAAGACACmΨGmΨACAACAG |
| | | ACGAACCAGACAAGACGAGCCCGCCCmΨGmΨmΨmΨGmΨGGCCCmΨGACCmΨmΨ |
| | | CGAGAGAAGAGAGGmΨGCmΨGGACAGCAGCAAmΨAmΨCAAGCCmΨAmΨGAACC |
| | | mΨGAmΨCGGCGmΨGGACCGGGGCGAGAACAmΨCCCmΨGCCGmΨGAmΨCGCCCm |
| | | ΨmΨACCGACCCCGAGGGAmΨGCCCmΨCmΨGAGCCGGmΨmΨmΨAAAGACAGCCm |
| | | ΨGGGCAACCCmΨACCCACAmΨCCmΨGAGAAmΨmΨGGCGAGmΨCCmΨACAAGGA |
| | | GAAGCAGAGAACCAmΨCCAGGCCAAGAAGGAGGmΨGGAGCAGCGGCGGGCmΨG |
| | | GCGGCmΨACmΨCCCGGAAGmΨACGCCAGCAAGGCCAAGAACCmΨGGCCGACGA |
| | | CAmΨGGmΨmΨAGAAAmΨACCGCCAGAGACCmΨCCmΨGmΨACmΨACGCmΨGmΨG |
| | | ACCCAGGACGCCAmΨGCmΨGAmΨCmΨmΨCGAGAACCmΨGAGCAGAGGCmΨmΨC |
| | | GGCAGACAGGGCAAGAGAACCmΨmΨCAmΨGGCCGAGAGACAGmΨACACCCGGA |
| | | mΨGGAGGACmΨGGCmΨGACCGCCAAGCmΨGGCCmΨACGAGGGCCmΨGCCCmΨC |
| | | mΨAAGACCmΨACCmΨGmΨCCAAGACCmΨmΨGGCACAGmΨACACCAGCAAGACA |
| | | mΨGCmΨCmΨAACmΨGCGGCmΨmΨCACAAmΨCACGAGCGCCGACmΨACGACCGG |
| | | GmΨGCmΨGGAGAAACmΨGAAGAAGACCGCCACAGGCmΨGGAmΨGACCACCAmΨ |
| | | mΨAACGGCAAGGAGCmΨGAAGGmΨGGAGGGCCAGAmΨCACCmΨACmΨACAACA |
| | | GGmΨACAAACGGCAGAACGmΨGGmΨGAAGGACCmΨGAGCGmΨGGAACmΨGGAm |
| | | ΨAGACmΨGAGCGAGGAAAGCGmΨAAACAAmΨGACAmΨCAGCAGCmΨGGACCAA |
| | | GGGCCGGAGCGGCGAGGCCCmΨGAGCCmΨGCmΨGAAGAAGAGAmΨmΨCmΨCCC |
| | | ACAGACCAGmΨGCAGGAGAAGmΨmΨCGmΨGmΨGmΨCmΨGAACmΨGCGGCmΨmΨ |
| | | CGAGACCCACGCCGACGAGCAAGCCGCCCmΨGAACAmΨCGCCCGGmΨCmΨmΨG |
| | | GCmΨmΨmΨmΨCCmΨGCGGAGCCAGGAGmΨACAAGAAGmΨACCAGACAAACAAG |
| | | ACCACAGGCAACACAGACAAGAGAGCCmΨmΨCGmΨCGAGACCmΨGGCAGAGCm |
| | | ΨmΨCmΨACAGAAAGAAGCmΨGAAGGAGGmΨGmΨGGAAGCCmΨGCCGmΨG |
| 19816 | DNA | CAGGAGATCAAGCGGATTAATAAAATTCGGAGAAGACTGGTGAAGGATTCTAA |
| | sequence | CACAAAGAAGGCTGGCAAGACAGGCCCTATGAAGACACTGCTGGTGAGAGTGA |
| | encoding | TGACACCCGACCTGAGAGAAAGACTGGAAAACCTGAGAAAGAAGCCTGAGAAT |
| | CasX 491 | ATCCCCCAGCCCATCAGCAACACAAGCCGGGCCAACCTGAATAAGCTGCTGAC |
| | | CGACTACACCGAAATGAAGAAGGCCATCCTGCACGTGTATTGGGAAGAGTTCC |
| | | AGAAAGACCCAGTCGGCCTGATGAGCAGAGTGGCTCAGCCTGCCAGCAAGAAG |
| | | ATCGATCAGAACAAGCTGAAGCCCGAAATGGACGAGAAGGGGAACCTGACAAC |
| | | CGCCGGCTTTGCCTGTAGCCAGTGCGGCCAGCCCCTGTTTGTGTACAAACTGG |
| | | AACAGGTGAGCGAAAAGGGCAAGGCTTACACGAATTACTTCGGCAGATGCAAC |
| | | GTGGCCGAGCACGAGAAGCTGATCCTGCTGGCCCAGCTGAAGCCTGAGAAGGA |
| | | TAGCGATGAGGCAGTGACATATTCCCTGGGCAAGTTCGGACAGCGGGCCCTGG |
| | | ATTTTTATTCCATTCATGTGACCAAGGAATCCACCCACCCCGTCAAGCCTCTT |
| | | GCCCAAATTGCCGGCAACAGATACGCCAGCGGCCCCGTGGGCAAGGCCCTGAG |
| | | CGACGCCTGTATGGGCACCATCGCCAGCTTCCTGTCTAAGTACCAGGACATTA |
| | | TCATCGAGCACCAGAAGGTGGTGAAGGGCAACCAGAAGAGACTGGAGAGCCTG |
| | | CGCGAGCTGGCCGGCAAGGAAAACCTGGAGTATCCTAGCGTGACCCTGCCTCC |
| | | TCAGCCTCATACAAAGGAGGGCGTGGATGCCTACAACGAAGTGATCGCCCGGG |
| | | TGCGGATGTGGGTGAACCTGAATCTGTGGCAGAAGCTGAAGCTGTCTAGAGAC |
| | | GACGCCAAGCCCCTGCTGAGACTGAAGGGCTTCCCCAGCTTCCCTCTGGTGGA |
| | | GAGACAGGCAAATGAAGTGGACTGGTGGGACATGGTGTGTAACGTGAAGAAGC |
| | | TGATCAATGAGAAGAAGGAGGACGGCAAAGTGTTCTGGCAGAATCTGGCCGGC |
| | | TACAAGCGTCAGGAGGCCCTGCGGCCCTACCTGAGCAGCGAGGAAGACAGAAA |
| | | GAAGGGCAAGAAGTTCGCCCGGTATCAGCTGGGGGACCTGCTGCTGCACCTCG |
| | | AGAAGAAGCACGGCGAAGACTGGGGGAAGGTGTACGATGAGGCCTGGGAGCGG |
| | | ATCGATAAGAAGGTGGAGGGCCTGAGCAAGCACATCAAGCTGGAGGAGGAACG |
| | | GAGATCTGAGGACGCCCAGAGCAAGGCCGCCCTGACCGACTGGCTGAGAGCCA |
| | | AGGCCAGCTTCGTCATCGAGGGGCTGAAGGAGGCCGACAAGGACGAGTTCTGC |
| | | CGGTGCGAACTGAAGCTGCAGAAGTGGTACGGAGATCTGAGAGGCAAACCTTT |
| | | CGCCATCGAGGCCGAGAACAGCATCCTGGACATCAGCGGCTTCAGCAAGCAGT |
| | | ACAACTGCGCCTTTATTTGGCAGAAGGACGGAGTGAAGAAGCTGAACCTGTAC |
| | | CTGATCATCAACTATTTCAAGGGCGGCAAGCTGAGATTCAAGAAGATCAAGCC |
| | | TGAAGCCTTCGAGGCCAACAGATTCTACACCGTGATTAACAAGAAAAGCGGAG |
| | | AGATCGTGCCAATGGAAGTGAACTTCAACTTCGACGACCCTAACCTGATCATC |
| | | CTGCCCCTGGCATTTGGCAAGCGGCAGGGCAGAGAGTTCATCTGGAACGACCT |
| | | GCTGTCTCTGGAGACCGGCAGCCTGAAGCTGGCCAACGGCAGAGTGATCGAGA |
| | | AGACACTGTACAACAGACGAACCAGACAAGACGAGCCCGCCCTGTTTGTGGCC |
| | | CTGACCTTCGAGAGAAGAGAGGTGCTGGACAGCAGCAATATCAAGCCTATGAA |
| | | CCTGATCGGCGTGGACCGGGGCGAGAACATCCCTGCCGTGATCGCCCTTACCG |
| | | ACCCCGAGGGATGCCCTCTGAGCCGGTTTAAAGACAGCCTGGGCAACCCTACC |
| | | CACATCCTGAGAATTGGCGAGTCCTACAAGGAGAAGCAGAGAACCATCCAGGC |
| | | CAAGAAGGAGGTGGAGCAGCGGCGGGCTGGCGGCTACTCCCGGAAGTACGCCA |
| | | GCAAGGCCAAGAACCTGGCCGACGACATGGTTAGAAATACCGCCAGAGACCTC |
| | | CTGTACTACGCTGTGACCCAGGACGCCATGCTGATCTTCGAGAACCTGAGCAG |
| | | AGGCTTCGGCAGACAGGGCAAGAGAACCTTCATGGCCGAGAGACAGTACACCC |
| | | GGATGGAGGACTGGCTGACCGCCAAGCTGGCCTACGAGGGCCTGTCTAAGACC |
| | | TACCTGTCCAAGACCTTGGCACAGTACACCAGCAAGACATGCTCTAACTGCGG |
| | | CTTCACAATCACGAGCGCCGACTACGACCGGGTGCTGGAGAAACTGAAGAAGA |
| | | CCGCCACAGGCTGGATGACCACCATTAACGGCAAGGAGCTGAAGGTGGAGGGC |
| | | CAGATCACCTACTACAACAGGTACAAACGGCAGAACGTGGTGAAGGACCTGAG |
| | | CGTGGAACTGGATAGACTGAGCGAGGAAAGCGTAAACAATGACATCAGCAGCT |
| | | GGACCAAGGGCCGGAGCGGCGAGGCCCTGAGCCTGCTGAAGAAGAGATTCTCC |
| | | CACAGACCAGTGCAGGAGAAGTTCGTGTGTCTGAACTGCGGCTTCGAGACCCA |
| | | CGCCGACGAGCAAGCCGCCCTGAACATCGCCCGGTCTTGGCTTTTCCTGCGGA |
| | | GCCAGGAGTACAAGAAGTACCAGACAAACAAGACCACAGGCAACACAGACAAG |
| | | AGAGCCTTCGTCGAGACCTGGCAGAGCTTCTACAGAAAGAAGCTGAAGGAGGT |
| | | GTGGAAGCCTGCCGTG |
| 19751 | DNA | CAGGAGATCAAGCGGATTAATAAAATTCGGAGAAGACTGGTGAAGGATTCTAA |
| | sequence | CACAAAGAAGGCTGGCAAGACAGGCCCTATGAAGACACTGCTGGTGAGAGTGA |
| | encoding | TGACACCCGACCTGAGAGAAAGACTGGAAAACCTGAGAAAGAAGCCTGAGAAT |
| | CasX 515 | ATCCCCCAGCCCATCAGCAACACAAGCCGGGCCAACCTGAATAAGCTGCTGAC |
| | | CGACTACACCGAAATGAAGAAGGCCATCCTGCACGTGTATTGGGAAGAGTTCC |
| | | AGAAAGACCCAGTCGGCCTGATGAGCAGAGTGGCTCAGCCTGCCAGCAAGAAG |
| | | ATCGATCAGAACAAGCTGAAGCCCGAAATGGACGAGAAGGGGAACCTGACAAC |
| | | CGCCGGCTTTGCCTGTAGCCAGTGCGGCCAGCCCCTGTTTGTGTACAAACTGG |
| | | AACAGGTGAGCGAAAAGGGCAAGGCTTACACGAATTACTTCGGCAGATGCAAC |
| | | GTGGCCGAGCACGAGAAGCTGATCCTGCTGGCCCAGCTGAAGCCTGAGAAGGA |
| | | TAGCGATGAGGCAGTGACATATTCCCTGGGCAAGTTCGGACAGCGGGCCCTGG |
| | | ATTTTTATTCCATTCATGTGACCAAGGAATCCACCCACCCCGTCAAGCCTCTT |
| | | GCCCAAATTGCCGGCAACAGATACGCCAGCGGCCCCGTGGGCAAGGCCCTGAG |
| | | CGACGCCTGTATGGGCACCATCGCCAGCTTCCTGTCTAAGTACCAGGACATTA |
| | | TCATCGAGCACCAGAAGGTGGTGAAGGGCAACCAGAAGAGACTGGAGAGCCTG |
| | | CGCGAGCTGGCCGGCAAGGAAAACCTGGAGTATCCTAGCGTGACCCTGCCTCC |
| | | TCAGCCTCATACAAAGGAGGGCGTGGATGCCTACAACGAAGTGATCGCCCGGG |
| | | TGCGGATGTGGGTGAACCTGAATCTGTGGCAGAAGCTGAAGCTGTCTAGAGAC |
| | | GACGCCAAGCCCCTGCTGAGACTGAAGGGCTTCCCCAGCTTCCCTCTGGTGGA |
| | | GAGACAGGCAAATGAAGTGGACTGGTGGGACATGGTGTGTAACGTGAAGAAGC |
| | | TGATCAATGAGAAGAAGGAGGACGGCAAAGTGTTCTGGCAGAATCTGGCCGGC |
| | | TACAAGCGTCAGGAGGCCCTGCGGCCCTACCTGAGCAGCGAGGAAGACAGAAA |
| | | GAAGGGCAAGAAGTTCGCCCGGTATCAGCTGGGGGACCTGCTGCTGCACCTCG |
| | | AGAAGAAGCACGGCGAAGACTGGGGGAAGGTGTACGATGAGGCCTGGGAGCGG |
| | | ATCGATAAGAAGGTGGAGGGCCTGAGCAAGCACATCAAGCTGGAGGAGGAACG |
| | | GAGATCTGAGGACGCCCAGAGCAAGGCCGCCCTGACCGACTGGCTGAGAGCCA |
| | | AGGCCAGCTTCGTCATCGAGGGGCTGAAGGAGGCCGACAAGGACGAGTTCTGC |
| | | CGGTGCGAACTGAAGCTGCAGAAGTGGTACGGAGATCTGAGAGGCAAACCTTT |
| | | CGCCATCGAGGCCGAGAACAGCATCCTGGACATCAGCGGCTTCAGCAAGCAGT |
| | | ACAACTGCGCCTTTATTTGGCAGAAGGACGGAGTGAAGAAGCTGAACCTGTAC |
| | | CTGATCATCAACTATTTCAAGGGCGGCAAGCTGAGATTCAAGAAGATCAAGCC |
| | | TGAAGCCTTCGAGGCCAACAGATTCTACACCGTGATTAACAAGAAAAGCGGAG |
| | | AGATCGTGCCAATGGAAGTGAACTTCAACTTCGACGACCCTAACCTGATCATC |
| | | CTGCCCCTGGCATTTGGCAAGCGGCAGGGCAGAGAGTTCATCTGGAACGACCT |
| | | GCTGTCTCTGGAGACCGGCAGCCTGAAGCTGGCCAACGGCAGAGTGATCGAGA |
| | | AGACACTGTACAACAGACGAACCAGACAAGACGAGCCCGCCCTGTTTGTGGCC |
| | | CTGACCTTCGAGAGAAGAGAGGTGCTGGACAGCAGCAATATCAAGCCTATGAA |
| | | CCTGATCGGCGTGGACCGGGGCGAGAACATCCCTGCCGTGATCGCCCTTACCG |
| | | ACCCCGAGGGATGCCCTCTGAGCCGGTTTAAAGACAGCCTGGGCAACCCTACC |
| | | CACATCCTGAGAATTGGCGAGTCCTACAAGGAGAAGCAGAGAACCATCCAGGC |
| | | CAAGAAGGAGGTGGAGCAGCGGCGGGCTGGCGGCTACTCCCGGAAGTACGCCA |
| | | GCAAGGCCAAGAACCTGGCCGACGACATGGTTAGAAATACCGCCAGAGACCTC |
| | | CTGTACTACGCTGTGACCCAGGACGCCATGCTGATCTTCGAGAACCTGAGCAG |
| | | AGGCTTCGGCAGACAGGGCAAGAGAACCTTCATGGCCGAGAGACAGTACACCC |
| | | GGATGGAGGACTGGCTGACCGCCAAGCTGGCCTACGAGGGCCTGCCCTCTAAG |
| | | ACCTACCTGTCCAAGACCTTGGCACAGTACACCAGCAAGACATGCTCTAACTG |
| | | CGGCTTCACAATCACGAGCGCCGACTACGACCGGGTGCTGGAGAAACTGAAGA |
| | | AGACCGCCACAGGCTGGATGACCACCATTAACGGCAAGGAGCTGAAGGTGGAG |
| | | GGCCAGATCACCTACTACAACAGGTACAAACGGCAGAACGTGGTGAAGGACCT |
| | | GAGCGTGGAACTGGATAGACTGAGCGAGGAAAGCGTAAACAATGACATCAGCA |
| | | GCTGGACCAAGGGCCGGAGCGGCGAGGCCCTGAGCCTGCTGAAGAAGAGATTC |
| | | TCCCACAGACCAGTGCAGGAGAAGTTCGTGTGTCTGAACTGCGGCTTCGAGAC |
| | | CCACGCCGACGAGCAAGCCGCCCTGAACATCGCCCGGTCTTGGCTTTTCCTGC |
| | | GGAGCCAGGAGTACAAGAAGTACCAGACAAACAAGACCACAGGCAACACAGAC |
| | | AAGAGAGCCTTCGTCGAGACCTGGCAGAGCTTCTACAGAAAGAAGCTGAAGGA |
| | | GGTGTGGAAGCCTGCCGTG |
| 19735 | DNA | CAGGAGATCAAGCGGATTAATAAAATTCGGAGAAGACTGGTGAAGGATTCTAA |
| | sequence | CACAAAGAAGGCTGGCAAGACACGGGGCCCTATGAAGACACTGCTGGTGAGAG |
| | encoding | TGATGACACCCGACCTGAGAGAAAGACTGGAAAACCTGAGAAAGAAGCCTGAG |
| | CasX 676 | AATATCCCCCAGCCCATCAGCAACACAAGCCGGGCCAACCTGAATAAGCTGCT |
| | | GACCGACTACACCGAAATGAAGAAGGCCATCCTGCACGTGTATTGGGAAGAGT |
| | | TCCAGAAAGACCCAGTCGGCCTGATGAGCAGAGTGGCTCAGCCTGCCAGCAAG |
| | | AAGATCGATCAGAACAAGCTGAAGCCCGAAATGGACGAGAAGGGGAACCTGAC |
| | | AACCGCCGGCTTTGCCTGTAGCCAGTGCGGCCAGCCCCTGTTTGTGTACAAAC |
| | | TGGAACAGGTGAGCGAAAAGGGCAAGGCTTACACGAATTACTTCGGCAGATGC |
| | | AACGTGGCCGAGCACGAGAAGCTGATCAAGCTGGCCCAGCTGAAGCCTGAGAA |
| | | GGATAGCGATGAGGCAGTGACATATTCCCTGGGCAAGTTCGGACAGCGGGCCC |
| | | TGGATTTTTATTCCATTCATGTGACCAAGGAATCCACCCACCCCGTCAAGCCT |
| | | CTTGCCCAAATTGCCGGCAACAGATACGCCTCCAGCCCCGTGGGCAAGGCCCT |
| | | GAGCGACGCCTGTATGGGCACCATCGCCAGCTTCCTGTCTAAGTACCAGGACA |
| | | TTATCATCGAGCACCAGAAGGTGGTGAAGGGCAACCAGAAGAGACTGGAGAGC |
| | | CTGCGCGAGCTGGCCGGCAAGGAAAACCTGGAGTATCCTAGCGTGACCCTGCC |
| | | TCCTCAGCCTCATACAAAGGAGGGCGTGGATGCCTACAACGAAGTGATCGCCC |
| | | GGGTGCGGATGTGGGTGAACCTGAATCTGTGGCAGAAGCTGAAGCTGTCTAGA |
| | | GACGACGCCAAGCCCCTGCTGAGACTGAAGGGCTTCCCCAGCTTCCCTCTGGT |
| | | GGAGAGACAGGCAAATGAAGTGGACTGGTGGGACATGGTGTGTAACGTGAAGA |
| | | AGCTGATCAATGAGAAGAAGGAGGACGGCAAAGTGTTCTGGCAGAATCTGGCC |
| | | GGCTACAAGCGTCAGGAGGCCCTGCGGCCCTACCTGAGCAGCGAGGAAGACAG |
| | | AAAGAAGGGCAAGAAGTTCGCCCGGTATCAGCTGGGGGACCTGCTGCTGCACC |
| | | TCGAGAAGAAGCACGGCGAAGACTGGGGGAAGGTGTACGATGAGGCCTGGGAG |
| | | CGGATCGATAAGAAGGTGGAGGGCCTGAGCAAGCACATCAAGCTGGAGGAGGA |
| | | ACGGAGATCTGAGGACGCCCAGAGCAAGGCCGCCCTGACCGACTGGCTGAGAG |
| | | CCAAGGCCAGCTTCGTCATCGAGGGGCTGAAGGAGGCCGACAAGGACGAGTTC |
| | | TGCCGGTGCGAACTGAAGCTGCAGAAGTGGTACGGAGATCTGAGAGGCAAACC |
| | | TTTCGCCATCGAGGCCGAGAACAGCATCCTGGACATCAGCGGCTTCAGCAAGC |
| | | AGTACAACTGCGCCTTTATTTGGCAGAAGGACGGAGTGAAGAAGCTGAACCTG |
| | | TACCTGATCATCAACTATTTCAAGGGCGGCAAGCTGAGATTCAAGAAGATCAA |
| | | GCCTGAAGCCTTCGAGGCCAACAGATTCTACACCGTGATTAACAAGAAAAGCG |
| | | GAGAGATCGTGCCAATGGAAGTGAACTTCAACTTCGACGACCCTAACCTGATC |
| | | ATCCTGCCCCTGGCATTTGGCAAGCGGCAGGGCAGAGAGTTCATCTGGAACGA |
| | | CCTGCTGTCTCTGGAGACCGGCAGCCTGAAGCTGGCCAACGGCAGAGTGATCG |
| | | AGAAGACACTGTACAACAGACGAACCAGACAAGACGAGCCCGCCCTGTTTGTG |
| | | GCCCTGACCTTCGAGAGAAGAGAGGTGCTGGACAGCAGCAATATCAAGCCTAT |
| | | GAACCTGATCGGCGTGGACCGGGGCGAGAACATCCCTGCCGTGATCGCCCTTA |
| | | CCGACCCCGAGGGATGCCCTCTGAGCCGGTTTAAAGACAGCCTGGGCAACCCT |
| | | ACCCACATCCTGAGAATTGGCGAGTCCTACAAGGAGAAGCAGAGAACCATCCA |
| | | GGCCAAGAAGGAGGTGGAGCAGCGGCGGGCTGGCGGCTACTCCCGGAAGTACG |
| | | CCAGCAAGGCCAAGAACCTGGCCGACGACATGGTTAGAAATACCGCCAGAGAC |
| | | CTCCTGTACTACGCTGTGACCCAGGACGCCATGCTGATCTTCGAGAACCTGAG |
| | | CAGAGGCTTCGGCAGACAGGGCAAGAGAACCTTCATGGCCGAGAGACAGTACA |
| | | CCCGGATGGAGGACTGGCTGACCGCCAAGCTGGCCTACGAGGGCCTGCCCTCT |
| | | AAGACCTACCTGTCCAAGACCTTGGCACAGTACACCAGCAAGACATGCTCTAA |
| | | CTGCGGCTTCACAATCACGAGCGCCGACTACGACCGGGTGCTGGAGAAACTGA |
| | | AGAAGACCGCCACAGGCTGGATGACCACCATTAACGGCAAGGAGCTGAAGGTG |
| | | GAGGGCCAGATCACCTACTACAACAGGTACAAACGGCAGAACGTGGTGAAGGA |
| | | CCTGAGCGTGGAACTGGATAGACTGAGCGAGGAAAGCGTAAACAATGACATCA |
| | | GCAGCTGGACCAAGGGCCGGAGCGGCGAGGCCCTGAGCCTGCTGAAGAAGAGA |
| | | TTCTCCCACAGACCAGTGCAGGAGAAGTTCGTGTGTCTGAACTGCGGCTTCGA |
| | | GACCCACGCCGACGAGCAAGCCGCCCTGAACATCGCCCGGTCTTGGCTTTTCC |
| | | TGCGGAGCCAGGAGTACAAGAAGTACCAGACAAACAAGACCACAGGCAACACA |
| | | GACAAGAGAGCCTTCGTCGAGACCTGGCAGAGCTTCTACAGAAAGAAGCTGAA |
| | | GGAGGTGTGGAAGCCTGCCGTG |
| 19752 | DNA | CAGGAGATCAAGCGGATTAATAAAATTCGGAGAAGACTGGTGAAGGATTCTAA |
| | sequence | CACAAAGAAGGCTGGCAAGACAGGCCCTATGAAGACACTGCTGGTGAGAGTGA |
| | encoding | TGACACCCGACCTGAGAGAAAGACTGGAAAACCTGAGAAAGAAGCCTGAGAAT |
| | CasX 812 | ATCCCCCAGCCCATCAGCAACACAAGCCGGGCCAACCTGAATAAGCTGCTGAC |
| | | CGACTACACCGAAATGAAGAAGGCCATCCTGCACGTGTATTGGGAAGAGTTCC |
| | | AGAAAGACCCAGTCGGCCTGATGAGCAGAGTGGCTCAGCCTGCCAGCAAGAAG |
| | | ATCGATCAGAACAAGCTGAAGCCCGAAATGGACGAGAAGGGGAACCTGACAAC |
| | | CGCCGGCTTTGCCTGTAGCCAGTGCGGCCAGCCCCTGTTTGTGTACAAACTGG |
| | | AACAGGTGAGCGAAAAGGGCAAGGCTTACACGAATTACTTCGGCAGATGCAAC |
| | | GTGGCCGAGCACGAGAAGCTGATCCTGCTGGCCCAGCTGAAGCCTGAGAAGGA |
| | | TAGCGATGAGGCAGTGACATATTCCCTGGGCAAGTTCGGACAGCGGGCCCTGG |
| | | ATTTTTATTCCATTCATGTGACCAAGGAATCCACCCACCCCGTCAAGCCTCTT |
| | | GCCCAAATTGCCGGCAACAGATACGCCAGCGGCCCCGTGGGCAAGGCCCTGAG |
| | | CGACGCCTGTATGGGCACCATCGCCAGCTTCCTGTCTAAGTACCAGGACATTA |
| | | TCATCGAGCACCAGAAGGTGGTGAAGGGCAACCAGAAGAGACTGGAGAGCCTG |
| | | CGCGAGCTGGCCGGCAAGGAAAACCTGGAGTATCCTAGCGTGACCCTGCCTCC |
| | | TCAGCCTCATACAAAGGAGGGCGTGGATGCCTACAACGAAGTGATCGCCCGGG |
| | | TGCGGATGTGGGTGAACCTGAATCTGTGGCAGAAGCTGAAGCTGTCTAGAGAC |
| | | GACGCCAAGCCCCTGCTGAGACTGAAGAAGTTCCCCAGCTTCCCTCTGGTGGA |
| | | GAGACAGGCAAATGAAGTGGACTGGTGGGACATGGTGTGTAACGTGAAGAAGC |
| | | TGATCAATGAGAAGAAGGAGGACGGCAAAGTGTTCTGGCAGAATCTGGCCGGC |
| | | TACAAGCGTCAGGAGGCCCTGCGGCCCTACCTGAGCAGCGAGGAAGACAGAAA |
| | | GAAGGGCAAGAAGTTCGCCCGGTATCAGCTGGGGGACCTGCTGCTGCACCTCG |
| | | AGAAGAAGCACGGCGAAGACTGGGGGAAGGTGTACGATGAGGCCTGGGAGCGG |
| | | ATCGATAAGAAGGTGGAGGGCCTGAGCAAGCACATCAAGCTGGAGGAGGAACG |
| | | GAGATCTGAGGACGCCCAGAGCAAGGCCGCCCTGACCGACTGGCTGAGAGCCA |
| | | AGGCCAGCTTCGTCATCGAGGGGCTGAAGGAGGCCGACAAGGACGAGTTCTGC |
| | | CGGTGCGAACTGAAGCTGCAGAAGTGGTACGGAGATCTGAGAGGCAAACCTTT |
| | | CGCCATCGAGGCCGAGAACAGCATCCTGGACATCAGCGGCTTCAGCAAGCAGT |
| | | ACAACTGCGCCTTTATTTGGCAGAAGGACGGAGTGAAGAAGCTGAACCTGTAC |
| | | CTGATCATCAACTATTTCAAGGGCGGCAAGCTGAGATTCAAGAAGATCAAGCC |
| | | TGAAGCCTTCGAGGCCAACAGATTCTACACCGTGATTAACAAGAAAAGCGGAG |
| | | AGATCGTGCCAATGGAAGTGAACTTCAACTTCGACGACCCTAACCTGATCATC |
| | | CTGCCCCTGGCATTTGGCAAGCGGCAGGGCAGAGAGTTCATCTGGAACGACCT |
| | | GCTGTCTCTGGAGACCGGCAGCCTGAAGCTGGCCAACGGCAGAGTGATCGAGA |
| | | AGACACTGTACAACAGACGAACCAGACAAGACGAGCCCGCCCTGTTTGTGGCC |
| | | CTGACCTTCGAGAGAAGAGAGGTGCTGGACAGCAGCAATATCAAGCCTATGAA |
| | | CCTGATCGGCGTGGACCGGGGCGAGAACATCCCTGCCGTGATCGCCCTTACCG |
| | | ACCCCGAGGGATGCCCTCTGAGCCGGTTTAAAGACAGCCTGGGCAACCCTACC |
| | | CACATCCTGAGAATTGGCGAGTCCTACAAGGAGAAGCAGAGAACCATCCAGGC |
| | | CAAGAAGGAGGTGGAGCAGCGGCGGGCTGGCGGCTACTCCCGGAAGTACGCCA |
| | | GCAAGGCCAAGAACCTGGCCGACGACATGGTTAGAAATACCGCCAGAGACCTC |
| | | CTGTACTACGCTGTGACCCAGGACGCCATGCTGATCTTCGAGAACCTGAGCAG |
| | | AGGCTTCGGCAGACAGGGCAAGAGAACCTTCATGGCCGAGAGACAGTACACCC |
| | | GGATGGAGGACTGGCTGACCGCCAAGCTGGCCTACGAGGGCCTGCCCTCTAAG |
| | | ACCTACCTGTCCAAGACCTTGGCACAGTACACCAGCAAGACATGCTCTAACTG |
| | | CGGCTTCACAATCACGAGCGCCGACTACGACCGGGTGCTGGAGAAACTGAAGA |
| | | AGACCGCCACAGGCTGGATGACCACCATTAACGGCAAGGAGCTGAAGGTGGAG |
| | | GGCCAGATCACCTACTACAACAGGTACAAACGGCAGAACGTGGTGAAGGACCT |
| | | GAGCGTGGAACTGGATAGACTGAGCGAGGAAAGCGTAAACAATGACATCAGCA |
| | | GCTGGACCAAGGGCCGGAGCGGCGAGGCCCTGAGCCTGCTGAAGAAGAGATTC |
| | | TCCCACAGACCAGTGCAGGAGAAGTTCGTGTGTCTGAACTGCGGCTTCGAGAC |
| | | CCACGCCGACGAGCAAGCCGCCCTGAACATCGCCCGGTCTTGGCTTTTCCTGC |
| | | GGAGCCAGGAGTACAAGAAGTACCAGACAAACAAGACCACAGGCAACACAGAC |
| | | AAGAGAGCCTTCGTCGAGACCTGGCAGAGCTTCTACAGAAAGAAGCTGAAGGA |
| | | GGTGTGGAAGCCTGCCGTG |

*‘mΨ’ = N1-methyl-pseudouridine

Various naturally-occurring or modified nucleosides may be used to produce mRNAs according to the present disclosure. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the mRNA comprises one or more nonstandard nucleotide residues. The nonstandard nucleotide residues may include, e.g., 5-methyl-cytidine (“5 mC”), N1-methyl-pseudouridine (“mų”), and/or 2-thio-uridine (“2sU”). In a particular embodiment, one or more of the uridine residues of the mRNA of the disclosure are replaced with N1-methyl-pseudouridine. See, e.g., U.S. Pat. No. 8,278,036 or WO2011012316, incorporated by reference herein, for a discussion of such residues and their incorporation into mRNA. In some embodiments, the mRNA encoding the engineered CasX protein of the disclosure has N1-methyl-pseudouridine nucleosides replacing one or more, or all uridines in the mRNA sequence portion encoding the engineered CasX protein. In some embodiments, the mRNA encoding the engineered CasX protein of the disclosure has N1-methyl-pseudouridine nucleosides replacing one or more, or all uridines in the sequence encoding the engineered CasX protein and one or more additional sequences of the mRNA; e.g., the UTR. In some embodiments, the mRNA encoding the engineered CasX protein of the disclosure has NI-methyl-pseudouridine nucleosides replacing one or more, or all uridines (from the 5′ end to the 3′ end) in the complete mRNA sequence. See, e.g., U.S. Pat. No. 8,278,036 or WO2011012316, incorporated by reference herein, for a discussion of such residues and their incorporation into mRNA. n some embodiments, the mRNA encoding CasX 515 has NI-methyl-pseudouridine nucleosides replacing one or more, or all uridines in the mRNA sequence. In some embodiments, the mRNA encoding CasX 812 has NI-methyl-pseudouridine nucleosides replacing one or more, or all uridines in the mRNA sequence. In some embodiments, the mRNA encoding CasX 491 has N1-methyl-pseudouridine nucleosides replacing one or more, or all uridines in the mRNA sequence. In some embodiments, the mRNA encoding CasX 676 has N1-methyl-pseudouridine nucleosides replacing one or more, or all uridines in the mRNA sequence.

In some embodiments, the mRNA sequence encoding the engineered CasX protein comprises the sequence of SEQ ID NO: 19817, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto. In some embodiments, the mRNA sequence encoding the engineered CasX protein comprises the sequence of SEQ ID NO: 19817. In some embodiments, the mRNA sequence encoding the engineered CasX protein consists of the sequence of SEQ ID NO: 19817. In some embodiments, the mRNA sequence encoding the engineered CasX protein comprises the sequence of SEQ ID NO: 19818, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto. In some embodiments, the mRNA sequence encoding the engineered CasX protein comprises the sequence of SEQ ID NO: 19818. In some embodiments, the mRNA sequence encoding the engineered CasX protein consists of the sequence of SEQ ID NO: 19818. In some embodiments, the mRNA sequence encoding the engineered CasX protein comprises the sequence of SEQ ID NO: 19819, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto. In some embodiments, the mRNA sequence encoding the engineered CasX protein comprises the sequence of SEQ ID NO: 19819. In some embodiments, the mRNA sequence encoding the engineered CasX protein consists of the sequence of SEQ ID NO: 19819. In some embodiments, the mRNA sequence encoding the engineered CasX protein comprises the sequence of SEQ ID NO: 19820, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto. In some embodiments, the mRNA sequence encoding the engineered CasX protein comprises the sequence of SEQ ID NO: 19820. In some embodiments, the mRNA sequence encoding the engineered CasX protein consists of the sequence of SEQ ID NO: 19820. In some embodiments, the DNA sequence encoding the engineered CasX protein is selected from the group consisting of SEQ ID NOS: 19735, 19751, 19752, and 19816.

### f. 5′ Untranslated Region (UTR)

The 5′ UTR of an mRNA molecule can be a key determinant of both the stability of the mRNA and how efficiently it is translated into protein. Specifically, the 5′ UTR, in conjunction with the 5′ cap structure, serves as a binding site and recruitment platform for the translation pre-initiation complex as well as additional regulatory proteins that may positively or negatively affect translation. Structures within the 5′ UTR can enhance translation by recruiting initiation factors or other protein or RNA factors, reduce translation by physically blocking ribosome binding and scanning, and contribute to the stability of the mRNA by affecting both hydrolysis and nuclease digestion. An exemplary 5′ UTR sequence for use in the mRNA of the disclosure is provided in Table 7. Table 7 lists the RNA sequence, RNA sequence with N1-methylpseudouridine substituted in place of uridine, and DNA sequence of the 5′ UTR.

TABLE 7
5′ UTR sequences

| SEQ ID | Nucleic Acid | |
|---|---|---|
| NO | Sequence* | Description |
| 19821 | AAAUAAGAGAGAAAAG | RNA sequence |
| | AAGAGUAAGAAGAAAU | of 5′ UTR |
| | AUAAGA | |
| 19822 | AAAmψAAGAGAGAAAA | N1-methylpseudouridine |
| | GAAGAGmψAAGAAGAA | substituted |
| | AmψAmψAAGA | RNA sequence |
| | | of 5′ UTR |
| 19823 | AAATAAGAGAGAAAAG | DNA sequence |
| | AAGAGTAAGAAGAAAT | encoding 5′ |
| | ATAAGA | UTR |

*‘mψ’ = N1-methyl-pseudouridine

### g. 5′ Cap

In some embodiments of the mRNA of the disclosure, the mRNA comprises a 5′ cap linked 5′ to the 5′ UTR of the mRNA sequence of any of the embodiments described herein. In some embodiments, the 5′ cap is a 7-methylguanylate cap. In some embodiments, the 5′ cap has the nucleic acid sequence of m7G (5) ppp (5′) mAG. In other embodiments, the 5′ cap has the nucleic acid sequence of m7G (5′) ppp (5′ (A,G (5′) ppp (5′) A or G (5′) ppp (5′) G.

### h. 3′ UTR

3′ UTR sequences can have a significant impact on mRNA stability and translation efficiency, and can determine both subcellular localization and tissue-specific expression. Factors influencing these properties include microRNA binding sites, AU-rich elements that recruit an array of RNA-binding proteins, Pumilio binding elements, and other binding sites for RNA-binding proteins. While many of these interactions with the 3′ UTR are known to negatively impact stability or expression, some can enhance translation. The effects of a 3′ UTR sequence can be highly cell-type specific due to differential expression of microRNAs and RNA binding proteins, which provides opportunities for engineering tissue-specific expression into a therapeutic mRNA. In some embodiments, the 3′ UTR for use in the mRNA of the disclosure is a mouse 3′ UTR. In some embodiments, the 3′ UTR is a mouse HBA gene 3′ UTR.

An exemplary 3′ UTR sequence for use in the mRNA of the disclosure is provided in Table 8. Table 8 lists the RNA sequence, RNA sequence with NI-methylpseudouridine substituted in place of uridine, and DNA sequence of the 3′ UTR.

TABLE 8
3′ UTR sequences

| SEQ ID | Nucleic Acid | |
|---|---|---|
| NO | Sequence* | Description |
| 19824 | GCUGCCUUCUGCGGGGC | RNA sequence of 3′ |
| | UUGCCUUCUGGCCAUGC | UTR |
| | CCUUCUUCUCUCCCUUG | |
| | CACCUGUACCUCUUGGU | |
| | CUUUGAAUAAAGCCUGA | |
| | GUAGGAAG | |
| 19825 | GCmψGCCmψmψCmψGCG | N1- |
| | GGGCmψmψGCCmψmψCm | methylpseudouridine |
| | ψGGCCAmψGCCCmψmψC | substituted RNA |
| | mψmψCmψCmψCCCmψmψ | sequence of 3′ UTR |
| | GCACCmψGmψACCmψCm | |
| | ψmψGGmψCmψmψmψGAA | |
| | mψAAAGCCmψGAGmψAG | |
| | GAAG | |
| 19740 | GCTGCCTTCTGCGGGGCT | DNA sequence |
| | TGCCTTCTGGCCATGCCC | encoding 3′ UTR |
| | TTCTTCTCTCCCTTGCAC | |
| | CTGTACCTCTTGGTCTTT | |
| | GAATAAAGCCTGAGTAGG | |
| | AAG | |

*‘mψ’ = N1-methyl-pseudouridine

In some embodiments, the 3′ UTR for use in the mRNA of the disclosure comprises the sequence of SEQ ID NO: 19824, or a sequence having at least about 50%, at least about 60%, at least about 70%, at least about 80%, at least about 90%, at least about 95%, or at least about 99% identity thereto. In some embodiments, the 3′ UTR comprises the sequence of SEQ ID NO: 19824. In some embodiments, the 3′ UTR consists of the sequence of SEQ ID NO: 19824. In some embodiments, the 3′ UTR comprises the sequence of SEQ ID NO: 19825, or a sequence having at least about 50%, at least about 60%, at least about 70%, at least about 80%, at least about 90%, at least about 95%, or at least about 99% identity thereto. In some embodiments, the 3′ UTR comprises the sequence of SEQ ID NO: 19825. In some embodiments, the 3′ UTR consists of the sequence of SEQ ID NO: 19825.

i. Poly (A) Sequence

Inclusion of a 3′ poly(A) tail in mRNA constructs can contribute to mRNA stability and translation efficiency. Generally, longer poly (A) tails are associated with increased mRNA stability, thereby allowing their translation and promoting high protein expression.

In some embodiments, the mRNAs of the disclosure comprise a poly (A) sequence. In some embodiments, the poly(A) sequence comprises at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 185, or at least about 190 adenine nucleotides. In some embodiments, the poly(A) sequence comprises about 40-190 adenine nucleotides. In some embodiments, the poly(A) sequence comprises about 50-150 adenine nucleotides. In some embodiments, the poly(A) sequence comprises about 70-90 adenine nucleotides. In some embodiments, the poly(A) sequence comprises about 78-82 adenine nucleotides. In some embodiments, the poly(A) sequence comprises 80 adenine nucleotides.

In some embodiments, the poly(A) sequence comprises the nucleic acid sequence of AAAAAAAAAAAAAAAAAAAAAAAAAAAAAAAAAAAAAAAAAAAAAAAAAAAA AAAAAAAAAAAAAAAAAAAAAAAAAAAA (SEQ ID NO: 19738). In some embodiments, the poly(A) sequence comprises 79 adenine nucleotides. In some embodiments, the poly(A) sequence comprises the nucleic acid sequence of AAAAAAAAAAAAAAAAAAAAAAAAAAAAAAAAAAAAAAAAAAAAAAAAAAAA AAAAAAAAAAAAAAAAAAAAAAAAAAA (SEQ ID NO: 19826).

j. mRNA Sequences of the Systems

The disclosure provides systems comprising mRNA sequences comprising the following components in 5′ to 3′ orientation; a 5′ UTR; a start codon; a sequence encoding a CasX protein; a stop codon; and a 3′ UTR. In some embodiments, the mRNA sequences comprise the following components in 5′ to 3′ orientation: a 5′ UTR; a start codon; an NLS; a sequence encoding a CasX protein; a stop codon; and a 3′ UTR. In some embodiments, the mRNA sequences comprise the following components in 5′ to 3′ orientation; a 5′ UTR; a start codon; a sequence encoding a CasX protein; an NLS; a stop codon; and a 3′ UTR. In some embodiments, the mRNA sequences comprise the following components in 5′ to 3′ orientation; a 5′ UTR; a start codon; an NLS; a sequence encoding a CasX protein; an NLS; a stop codon; and a 3′ UTR. In other cases, the mRNA comprises the following components in 5′ to 3′ orientation; a 5′ cap; a 5′ UTR; a start codon; an NLS; a sequence encoding a CasX protein; a stop codon; a 3′ UTR; and a poly (A) sequence. In some embodiments, the mRNA comprises the following components in 5′ to 3′ orientation; a 5′ cap; a 5′ UTR; a start codon; a sequence encoding a CasX protein; an NLS; a stop codon; a 3′ UTR; and a poly (A) sequence. In some embodiments, the mRNA comprises the following components in 5′ to 3′ orientation; a 5′ cap; a 5′ UTR; a start codon; an NLS; a sequence encoding a CasX protein; an NLS; a stop codon; a 3′ UTR; and a poly (A) sequence. In other cases, the mRNA comprises the following components in 5′ to 3′ orientation: a 5′ cap; a 5′ UTR; a start codon; a sequence encoding a CasX protein; a stop codon; a 3′ UTR; and a poly (A) sequence. In some embodiments, the mRNA has N1-methyl-pseudouridine nucleosides replacing one or more, or all uridines in the complete mRNA sequence.

In some embodiments, the disclosure provides an mRNA sequence comprising the sequence of SEQ ID NO: 19830 or SEQ ID NO: 19834, or a sequence having at least about 70%, at least about 80%, at least about 90%, at least about 95%, at least about 95%, at least about 96%, at least about 97%, at least about 98%, or at least about 99% sequence identity thereto. In some embodiments, the disclosure provides an mRNA sequence comprising the sequence of SEQ ID NO: 19831 or SEQ ID NO: 19835, or a sequence having at least about 70%, at least about 80%, at least about 90%, at least about 95%, at least about 95%, at least about 96%, at least about 97%, at least about 98%, or at least about 99% sequence identity thereto. In some embodiments, the disclosure provides an mRNA sequence comprising the sequence of SEQ ID NO: 19832 or SEQ ID NO: 19836, or a sequence having at least about 70%, at least about 80%, at least about 90%, at least about 95%, at least about 95%, at least about 96%, at least about 97%, at least about 98%, or at least about 99% sequence identity thereto. In some embodiments, the disclosure provides an mRNA sequence comprising the sequence of SEQ ID NO: 19833 or SEQ ID NO: 19837, or a sequence having at least about 70%, at least about 80%, at least about 90%, at least about 95%, at least about 95%, at least about 96%, at least about 97%, at least about 98%, or at least about 99% sequence identity thereto.

Exemplary full-length mRNA sequences are provided in Table 9. Table 9 lists the RNA sequences, RNA sequences with N1-methylpseudouridine substituted in place of uridine, and DNA sequences of the mRNAs. The sequences in Table 9 include, from 5′ to 3′, AGG nucleotides 5′ of the 5′ UTR, a 5′ UTR, GCCACC (SEQ ID NO: 19829) nucleotides, a start codon, a sequence encoding a c-MYC NLS, a sequence encoding a linker, a sequence encoding CasX, a sequence encoding a linker, a sequence encoding a c-MYC NLS, a stop codon, a 3′ UTR, and a sequence corresponding to a partial XbaI restriction site (UCUAG, SEQ ID NO: 19827; myCmyAG, SEQ ID NO: 19828; or TCTAG, SEQ ID NO: 19741, for RNA, N1-methylpseudouridine substituted RNA, and DNA sequences, respectively).

TABLE 9
Exemplary full-length mRNA sequences encoding CasX

| SEQ | | |
|---|---|---|
| ID NO | Nucleotide Sequence* | Description |
| 19830 | AGGAAAUAAGAGAGAAAAGAAGAGUAAGAAGAAAUAUAAGAGCCACCAUGGCCCC | Full-length |
| | UGCUGCCAAGAGAGUGAAGCUGGAUAGCAGACAGGAGAUCAAGCGGAUUAAUAAA | RNA |
| | AUUCGGAGAAGACUGGUGAAGGAUUCUAACACAAAGAAGGCUGGCAAGACAGGCC | sequence |
| | CUAUGAAGACACUGCUGGUGAGAGUGAUGACACCCGACCUGAGAGAAAGACUGGA | of mRNA |
| | AAACCUGAGAAAGAAGCCUGAGAAUAUCCCCCAGCCCAUCAGCAACACAAGCCGG | encoding |
| | GCCAACCUGAAUAAGCUGCUGACCGACUACACCGAAAUGAAGAAGGCCAUCCUGC | CasX 491 |
| | ACGUGUAUUGGGAAGAGUUCCAGAAAGACCCAGUCGGCCUGAUGAGCAGAGUGGC | |
| | UCAGCCUGCCAGCAAGAAGAUCGAUCAGAACAAGCUGAAGCCCGAAAUGGACGAG | |
| | AAGGGGAACCUGACAACCGCCGGCUUUGCCUGUAGCCAGUGCGGCCAGCCCCUGU | |
| | UUGUGUACAAACUGGAACAGGUGAGCGAAAAGGGCAAGGCUUACACGAAUUACUU | |
| | CGGCAGAUGCAACGUGGCCGAGCACGAGAAGCUGAUCCUGCUGGCCCAGCUGAAG | |
| | CCUGAGAAGGAUAGCGAUGAGGCAGUGACAUAUUCCCUGGGCAAGUUCGGACAGC | |
| | GGGCCCUGGAUUUUUAUUCCAUUCAUGUGACCAAGGAAUCCACCCACCCCGUCAA | |
| | GCCUCUUGCCCAAAUUGCCGGCAACAGAUACGCCAGCGGCCCCGUGGGCAAGGCC | |
| | CUGAGCGACGCCUGUAUGGGCACCAUCGCCAGCUUCCUGUCUAAGUACCAGGACA | |
| | UUAUCAUCGAGCACCAGAAGGUGGUGAAGGGCAACCAGAAGAGACUGGAGAGCCU | |
| | GCGCGAGCUGGCCGGCAAGGAAAACCUGGAGUAUCCUAGCGUGACCCUGCCUCCU | |
| | CAGCCUCAUACAAAGGAGGGCGUGGAUGCCUACAACGAAGUGAUCGCCCGGGUGC | |
| | GGAUGUGGGUGAACCUGAAUCUGUGGCAGAAGCUGAAGCUGUCUAGAGACGACGC | |
| | CAAGCCCCUGCUGAGACUGAAGGGCUUCCCCAGCUUCCCUCUGGUGGAGAGACAG | |
| | GCAAAUGAAGUGGACUGGUGGGACAUGGUGUGUAACGUGAAGAAGCUGAUCAAUG | |
| | AGAAGAAGGAGGACGGCAAAGUGUUCUGGCAGAAUCUGGCCGGCUACAAGCGUCA | |
| | GGAGGCCCUGCGGCCCUACCUGAGCAGCGAGGAAGACAGAAAGAAGGGCAAGAAG | |
| | UUCGCCCGGUAUCAGCUGGGGGACCUGCUGCUGCACCUCGAGAAGAAGCACGGCG | |
| | AAGACUGGGGGAAGGUGUACGAUGAGGCCUGGGAGCGGAUCGAUAAGAAGGUGGA | |
| | GGGCCUGAGCAAGCACAUCAAGCUGGAGGAGGAACGGAGAUCUGAGGACGCCCAG | |
| | AGCAAGGCCGCCCUGACCGACUGGCUGAGAGCCAAGGCCAGCUUCGUCAUCGAGG | |
| | GGCUGAAGGAGGCCGACAAGGACGAGUUCUGCCGGUGCGAACUGAAGCUGCAGAA | |
| | GUGGUACGGAGAUCUGAGAGGCAAACCUUUCGCCAUCGAGGCCGAGAACAGCAUC | |
| | CUGGACAUCAGCGGCUUCAGCAAGCAGUACAACUGCGCCUUUAUUUGGCAGAAGG | |
| | ACGGAGUGAAGAAGCUGAACCUGUACCUGAUCAUCAACUAUUUCAAGGGCGGCAA | |
| | GCUGAGAUUCAAGAAGAUCAAGCCUGAAGCCUUCGAGGCCAACAGAUUCUACACC | |
| | GUGAUUAACAAGAAAAGCGGAGAGAUCGUGCCAAUGGAAGUGAACUUCAACUUCG | |
| | ACGACCCUAACCUGAUCAUCCUGCCCCUGGCAUUUGGCAAGCGGCAGGGCAGAGA | |
| | GUUCAUCUGGAACGACCUGCUGUCUCUGGAGACCGGCAGCCUGAAGCUGGCCAAC | |
| | GGCAGAGUGAUCGAGAAGACACUGUACAACAGACGAACCAGACAAGACGAGCCCG | |
| | CCCUGUUUGUGGCCCUGACCUUCGAGAGAAGAGAGGUGCUGGACAGCAGCAAUAU | |
| | CAAGCCUAUGAACCUGAUCGGCGUGGACCGGGGCGAGAACAUCCCUGCCGUGAUC | |
| | GCCCUUACCGACCCCGAGGGAUGCCCUCUGAGCCGGUUUAAAGACAGCCUGGGCA | |
| | ACCCUACCCACAUCCUGAGAAUUGGCGAGUCCUACAAGGAGAAGCAGAGAACCAU | |
| | CCAGGCCAAGAAGGAGGUGGAGCAGCGGCGGGCUGGCGGCUACUCCCGGAAGUAC | |
| | GCCAGCAAGGCCAAGAACCUGGCCGACGACAUGGUUAGAAAUACCGCCAGAGACC | |
| | UCCUGUACUACGCUGUGACCCAGGACGCCAUGCUGAUCUUCGAGAACCUGAGCAG | |
| | AGGCUUCGGCAGACAGGGCAAGAGAACCUUCAUGGCCGAGAGACAGUACACCCGG | |
| | AUGGAGGACUGGCUGACCGCCAAGCUGGCCUACGAGGGCCUGUCUAAGACCUACC | |
| | UGUCCAAGACCUUGGCACAGUACACCAGCAAGACAUGCUCUAACUGCGGCUUCAC | |
| | AAUCACGAGCGCCGACUACGACCGGGUGCUGGAGAAACUGAAGAAGACCGCCACA | |
| | GGCUGGAUGACCACCAUUAACGGCAAGGAGCUGAAGGUGGAGGGCCAGAUCACCU | |
| | ACUACAACAGGUACAAACGGCAGAACGUGGUGAAGGACCUGAGCGUGGAACUGGA | |
| | UAGACUGAGCGAGGAAAGCGUAAACAAUGACAUCAGCAGCUGGACCAAGGGCCGG | |
| | AGCGGCGAGGCCCUGAGCCUGCUGAAGAAGAGAUUCUCCCACAGACCAGUGCAGG | |
| | AGAAGUUCGUGUGUCUGAACUGCGGCUUCGAGACCCACGCCGACGAGCAAGCCGC | |
| | CCUGAACAUCGCCCGGUCUUGGCUUUUCCUGCGGAGCCAGGAGUACAAGAAGUAC | |
| | CAGACAAACAAGACCACAGGCAACACAGACAAGAGAGCCUUCGUCGAGACCUGGC | |
| | AGAGCUUCUACAGAAAGAAGCUGAAGGAGGUGUGGAAGCCUGCCGUGGGAAGCCC | |
| | CGCUGCCAAGAGAGUGAAGCUGGACUAAGCUGCCUUCUGCGGGGCUUGCCUUCUG | |
| | GCCAUGCCCUUCUUCUCUCCCUUGCACCUGUACCUCUUGGUCUUUGAAUAAAGCC | |
| | UGAGUAGGAAGUCUAGAAAAAAAAAAAAAAAAAAAAAAAAAAAAAAAAAAAAAAA | |
| | AAAAAAAAAAAAAAAAAAAAAAAAAAAAAAAAAAAAAAAA | |
| 19831 | AGGAAAUAAGAGAGAAAAGAAGAGUAAGAAGAAAUAUAAGAGCCACCAUGGCCCC | Full-length |
| | UGCUGCCAAGAGAGUGAAGCUGGAUAGCAGACAGGAGAUCAAGCGGAUUAAUAAA | RNA |
| | AUUCGGAGAAGACUGGUGAAGGAUUCUAACACAAAGAAGGCUGGCAAGACAGGCC | sequence |
| | CUAUGAAGACACUGCUGGUGAGAGUGAUGACACCCGACCUGAGAGAAAGACUGGA | of mRNA |
| | AAACCUGAGAAAGAAGCCUGAGAAUAUCCCCCAGCCCAUCAGCAACACAAGCCGG | encoding |
| | GCCAACCUGAAUAAGCUGCUGACCGACUACACCGAAAUGAAGAAGGCCAUCCUGC | CasX 515 |
| | ACGUGUAUUGGGAAGAGUUCCAGAAAGACCCAGUCGGCCUGAUGAGCAGAGUGGC | |
| | UCAGCCUGCCAGCAAGAAGAUCGAUCAGAACAAGCUGAAGCCCGAAAUGGACGAG | |
| | AAGGGGAACCUGACAACCGCCGGCUUUGCCUGUAGCCAGUGCGGCCAGCCCCUGU | |
| | UUGUGUACAAACUGGAACAGGUGAGCGAAAAGGGCAAGGCUUACACGAAUUACUU | |
| | CGGCAGAUGCAACGUGGCCGAGCACGAGAAGCUGAUCCUGCUGGCCCAGCUGAAG | |
| | CCUGAGAAGGAUAGCGAUGAGGCAGUGACAUAUUCCCUGGGCAAGUUCGGACAGC | |
| | GGGCCCUGGAUUUUUAUUCCAUUCAUGUGACCAAGGAAUCCACCCACCCCGUCAA | |
| | GCCUCUUGCCCAAAUUGCCGGCAACAGAUACGCCAGCGGCCCCGUGGGCAAGGCC | |
| | CUGAGCGACGCCUGUAUGGGCACCAUCGCCAGCUUCCUGUCUAAGUACCAGGACA | |
| | UUAUCAUCGAGCACCAGAAGGUGGUGAAGGGCAACCAGAAGAGACUGGAGAGCCU | |
| | GCGCGAGCUGGCCGGCAAGGAAAACCUGGAGUAUCCUAGCGUGACCCUGCCUCCU | |
| | CAGCCUCAUACAAAGGAGGGCGUGGAUGCCUACAACGAAGUGAUCGCCCGGGUGC | |
| | GGAUGUGGGUGAACCUGAAUCUGUGGCAGAAGCUGAAGCUGUCUAGAGACGACGC | |
| | CAAGCCCCUGCUGAGACUGAAGGGCUUCCCCAGCUUCCCUCUGGUGGAGAGACAG | |
| | GCAAAUGAAGUGGACUGGUGGGACAUGGUGUGUAACGUGAAGAAGCUGAUCAAUG | |
| | AGAAGAAGGAGGACGGCAAAGUGUUCUGGCAGAAUCUGGCCGGCUACAAGCGUCA | |
| | GGAGGCCCUGCGGCCCUACCUGAGCAGCGAGGAAGACAGAAAGAAGGGCAAGAAG | |
| | UUCGCCCGGUAUCAGCUGGGGGACCUGCUGCUGCACCUCGAGAAGAAGCACGGCG | |
| | AAGACUGGGGGAAGGUGUACGAUGAGGCCUGGGAGCGGAUCGAUAAGAAGGUGGA | |
| | GGGCCUGAGCAAGCACAUCAAGCUGGAGGAGGAACGGAGAUCUGAGGACGCCCAG | |
| | AGCAAGGCCGCCCUGACCGACUGGCUGAGAGCCAAGGCCAGCUUCGUCAUCGAGG | |
| | GGCUGAAGGAGGCCGACAAGGACGAGUUCUGCCGGUGCGAACUGAAGCUGCAGAA | |
| | GUGGUACGGAGAUCUGAGAGGCAAACCUUUCGCCAUCGAGGCCGAGAACAGCAUC | |
| | CUGGACAUCAGCGGCUUCAGCAAGCAGUACAACUGCGCCUUUAUUUGGCAGAAGG | |
| | ACGGAGUGAAGAAGCUGAACCUGUACCUGAUCAUCAACUAUUUCAAGGGCGGCAA | |
| | GCUGAGAUUCAAGAAGAUCAAGCCUGAAGCCUUCGAGGCCAACAGAUUCUACACC | |
| | GUGAUUAACAAGAAAAGCGGAGAGAUCGUGCCAAUGGAAGUGAACUUCAACUUCG | |
| | ACGACCCUAACCUGAUCAUCCUGCCCCUGGCAUUUGGCAAGCGGCAGGGCAGAGA | |
| | GUUCAUCUGGAACGACCUGCUGUCUCUGGAGACCGGCAGCCUGAAGCUGGCCAAC | |
| | GGCAGAGUGAUCGAGAAGACACUGUACAACAGACGAACCAGACAAGACGAGCCCG | |
| | CCCUGUUUGUGGCCCUGACCUUCGAGAGAAGAGAGGUGCUGGACAGCAGCAAUAU | |
| | CAAGCCUAUGAACCUGAUCGGCGUGGACCGGGGCGAGAACAUCCCUGCCGUGAUC | |
| | GCCCUUACCGACCCCGAGGGAUGCCCUCUGAGCCGGUUUAAAGACAGCCUGGGCA | |
| | ACCCUACCCACAUCCUGAGAAUUGGCGAGUCCUACAAGGAGAAGCAGAGAACCAU | |
| | CCAGGCCAAGAAGGAGGUGGAGCAGCGGCGGGCUGGCGGCUACUCCCGGAAGUAC | |
| | GCCAGCAAGGCCAAGAACCUGGCCGACGACAUGGUUAGAAAUACCGCCAGAGACC | |
| | UCCUGUACUACGCUGUGACCCAGGACGCCAUGCUGAUCUUCGAGAACCUGAGCAG | |
| | AGGCUUCGGCAGACAGGGCAAGAGAACCUUCAUGGCCGAGAGACAGUACACCCGG | |
| | AUGGAGGACUGGCUGACCGCCAAGCUGGCCUACGAGGGCCUGCCCUCUAAGACCU | |
| | ACCUGUCCAAGACCUUGGCACAGUACACCAGCAAGACAUGCUCUAACUGCGGCUU | |
| | CACAAUCACGAGCGCCGACUACGACCGGGUGCUGGAGAAACUGAAGAAGACCGCC | |
| | ACAGGCUGGAUGACCACCAUUAACGGCAAGGAGCUGAAGGUGGAGGGCCAGAUCA | |
| | CCUACUACAACAGGUACAAACGGCAGAACGUGGUGAAGGACCUGAGCGUGGAACU | |
| | GGAUAGACUGAGCGAGGAAAGCGUAAACAAUGACAUCAGCAGCUGGACCAAGGGC | |
| | CGGAGCGGCGAGGCCCUGAGCCUGCUGAAGAAGAGAUUCUCCCACAGACCAGUGC | |
| | AGGAGAAGUUCGUGUGUCUGAACUGCGGCUUCGAGACCCACGCCGACGAGCAAGC | |
| | CGCCCUGAACAUCGCCCGGUCUUGGCUUUUCCUGCGGAGCCAGGAGUACAAGAAG | |
| | UACCAGACAAACAAGACCACAGGCAACACAGACAAGAGAGCCUUCGUCGAGACCU | |
| | GGCAGAGCUUCUACAGAAAGAAGCUGAAGGAGGUGUGGAAGCCUGCCGUGGGAAG | |
| | CCCCGCUGCCAAGAGAGUGAAGCUGGACUAAGCUGCCUUCUGCGGGGCUUGCCUU | |
| | CUGGCCAUGCCCUUCUUCUCUCCCUUGCACCUGUACCUCUUGGUCUUUGAAUAAA | |
| | GCCUGAGUAGGAAGUCUAGAAAAAAAAAAAAAAAAAAAAAAAAAAAAAAAAAAAA | |
| | AAAAAAAAAAAAAAAAAAAAAAAAAAAAAAAAAAAAAAAAAAA | |
| 19832 | AGGAAAUAAGAGAGAAAAGAAGAGUAAGAAGAAAUAUAAGAGCCACCAUGGCCCC | Full-length |
| | UGCUGCCAAGAGAGUGAAGCUGGAUAGCAGACAGGAGAUCAAGCGGAUUAAUAAA | RNA |
| | AUUCGGAGAAGACUGGUGAAGGAUUCUAACACAAAGAAGGCUGGCAAGACACGGG | sequence |
| | GCCCUAUGAAGACACUGCUGGUGAGAGUGAUGACACCCGACCUGAGAGAAAGACU | encoding |
| | GGAAAACCUGAGAAAGAAGCCUGAGAAUAUCCCCCAGCCCAUCAGCAACACAAGC | CasX 676 |
| | CGGGCCAACCUGAAUAAGCUGCUGACCGACUACACCGAAAUGAAGAAGGCCAUCC | |
| | UGCACGUGUAUUGGGAAGAGUUCCAGAAAGACCCAGUCGGCCUGAUGAGCAGAGU | |
| | GGCUCAGCCUGCCAGCAAGAAGAUCGAUCAGAACAAGCUGAAGCCCGAAAUGGAC | |
| | GAGAAGGGGAACCUGACAACCGCCGGCUUUGCCUGUAGCCAGUGCGGCCAGCCCC | |
| | UGUUUGUGUACAAACUGGAACAGGUGAGCGAAAAGGGCAAGGCUUACACGAAUUA | |
| | CUUCGGCAGAUGCAACGUGGCCGAGCACGAGAAGCUGAUCAAGCUGGCCCAGCUG | |
| | AAGCCUGAGAAGGAUAGCGAUGAGGCAGUGACAUAUUCCCUGGGCAAGUUCGGAC | |
| | AGCGGGCCCUGGAUUUUUAUUCCAUUCAUGUGACCAAGGAAUCCACCCACCCCGU | |
| | CAAGCCUCUUGCCCAAAUUGCCGGCAACAGAUACGCCUCCAGCCCCGUGGGCAAG | |
| | GCCCUGAGCGACGCCUGUAUGGGCACCAUCGCCAGCUUCCUGUCUAAGUACCAGG | |
| | ACAUUAUCAUCGAGCACCAGAAGGUGGUGAAGGGCAACCAGAAGAGACUGGAGAG | |
| | CCUGCGCGAGCUGGCCGGCAAGGAAAACCUGGAGUAUCCUAGCGUGACCCUGCCU | |
| | CCUCAGCCUCAUACAAAGGAGGGCGUGGAUGCCUACAACGAAGUGAUCGCCCGGG | |
| | UGCGGAUGUGGGUGAACCUGAAUCUGUGGCAGAAGCUGAAGCUGUCUAGAGACGA | |
| | CGCCAAGCCCCUGCUGAGACUGAAGGGCUUCCCCAGCUUCCCUCUGGUGGAGAGA | |
| | CAGGCAAAUGAAGUGGACUGGUGGGACAUGGUGUGUAACGUGAAGAAGCUGAUCA | |
| | AUGAGAAGAAGGAGGACGGCAAAGUGUUCUGGCAGAAUCUGGCCGGCUACAAGCG | |
| | UCAGGAGGCCCUGCGGCCCUACCUGAGCAGCGAGGAAGACAGAAAGAAGGGCAAG | |
| | AAGUUCGCCCGGUAUCAGCUGGGGGACCUGCUGCUGCACCUCGAGAAGAAGCACG | |
| | GCGAAGACUGGGGGAAGGUGUACGAUGAGGCCUGGGAGCGGAUCGAUAAGAAGGU | |
| | GGAGGGCCUGAGCAAGCACAUCAAGCUGGAGGAGGAACGGAGAUCUGAGGACGCC | |
| | CAGAGCAAGGCCGCCCUGACCGACUGGCUGAGAGCCAAGGCCAGCUUCGUCAUCG | |
| | AGGGGCUGAAGGAGGCCGACAAGGACGAGUUCUGCCGGUGCGAACUGAAGCUGCA | |
| | GAAGUGGUACGGAGAUCUGAGAGGCAAACCUUUCGCCAUCGAGGCCGAGAACAGC | |
| | AUCCUGGACAUCAGCGGCUUCAGCAAGCAGUACAACUGCGCCUUUAUUUGGCAGA | |
| | AGGACGGAGUGAAGAAGCUGAACCUGUACCUGAUCAUCAACUAUUUCAAGGGCGG | |
| | CAAGCUGAGAUUCAAGAAGAUCAAGCCUGAAGCCUUCGAGGCCAACAGAUUCUAC | |
| | ACCGUGAUUAACAAGAAAAGCGGAGAGAUCGUGCCAAUGGAAGUGAACUUCAACU | |
| | UCGACGACCCUAACCUGAUCAUCCUGCCCCUGGCAUUUGGCAAGCGGCAGGGCAG | |
| | AGAGUUCAUCUGGAACGACCUGCUGUCUCUGGAGACCGGCAGCCUGAAGCUGGCC | |
| | AACGGCAGAGUGAUCGAGAAGACACUGUACAACAGACGAACCAGACAAGACGAGC | |
| | CCGCCCUGUUUGUGGCCCUGACCUUCGAGAGAAGAGAGGUGCUGGACAGCAGCAA | |
| | UAUCAAGCCUAUGAACCUGAUCGGCGUGGACCGGGGCGAGAACAUCCCUGCCGUG | |
| | AUCGCCCUUACCGACCCCGAGGGAUGCCCUCUGAGCCGGUUUAAAGACAGCCUGG | |
| | GCAACCCUACCCACAUCCUGAGAAUUGGCGAGUCCUACAAGGAGAAGCAGAGAAC | |
| | CAUCCAGGCCAAGAAGGAGGUGGAGCAGCGGCGGGCUGGCGGCUACUCCCGGAAG | |
| | UACGCCAGCAAGGCCAAGAACCUGGCCGACGACAUGGUUAGAAAUACCGCCAGAG | |
| | ACCUCCUGUACUACGCUGUGACCCAGGACGCCAUGCUGAUCUUCGAGAACCUGAG | |
| | CAGAGGCUUCGGCAGACAGGGCAAGAGAACCUUCAUGGCCGAGAGACAGUACACC | |
| | CGGAUGGAGGACUGGCUGACCGCCAAGCUGGCCUACGAGGGCCUGCCCUCUAAGA | |
| | CCUACCUGUCCAAGACCUUGGCACAGUACACCAGCAAGACAUGCUCUAACUGCGG | |
| | CUUCACAAUCACGAGCGCCGACUACGACCGGGUGCUGGAGAAACUGAAGAAGACC | |
| | GCCACAGGCUGGAUGACCACCAUUAACGGCAAGGAGCUGAAGGUGGAGGGCCAGA | |
| | UCACCUACUACAACAGGUACAAACGGCAGAACGUGGUGAAGGACCUGAGCGUGGA | |
| | ACUGGAUAGACUGAGCGAGGAAAGCGUAAACAAUGACAUCAGCAGCUGGACCAAG | |
| | GGCCGGAGCGGCGAGGCCCUGAGCCUGCUGAAGAAGAGAUUCUCCCACAGACCAG | |
| | UGCAGGAGAAGUUCGUGUGUCUGAACUGCGGCUUCGAGACCCACGCCGACGAGCA | |
| | AGCCGCCCUGAACAUCGCCCGGUCUUGGCUUUUCCUGCGGAGCCAGGAGUACAAG | |
| | AAGUACCAGACAAACAAGACCACAGGCAACACAGACAAGAGAGCCUUCGUCGAGA | |
| | CCUGGCAGAGCUUCUACAGAAAGAAGCUGAAGGAGGUGUGGAAGCCUGCCGUGGG | |
| | AAGCCCCGCUGCCAAGAGAGUGAAGCUGGACUAAGCUGCCUUCUGCGGGGCUUGC | |
| | CUUCUGGCCAUGCCCUUCUUCUCUCCCUUGCACCUGUACCUCUUGGUCUUUGAAU | |
| | AAAGCCUGAGUAGGAAGUCUAGAAAAAAAAAAAAAAAAAAAAAAAAAAAAAAAAA | |
| | AAAAAAAAAAAAAAAAAAAAAAAAAAAAAAAAAAAAAAAAAAAAAA | |
| 19833 | AGGAAAUAAGAGAGAAAAGAAGAGUAAGAAGAAAUAUAAGAGCCACCAUGGCCCC | Full-length |
| | UGCUGCCAAGAGAGUGAAGCUGGAUAGCAGACAGGAGAUCAAGCGGAUUAAUAAA | RNA |
| | AUUCGGAGAAGACUGGUGAAGGAUUCUAACACAAAGAAGGCUGGCAAGACAGGCC | sequence |
| | CUAUGAAGACACUGCUGGUGAGAGUGAUGACACCCGACCUGAGAGAAAGACUGGA | of mRNA |
| | AAACCUGAGAAAGAAGCCUGAGAAUAUCCCCCAGCCCAUCAGCAACACAAGCCGG | encoding |
| | GCCAACCUGAAUAAGCUGCUGACCGACUACACCGAAAUGAAGAAGGCCAUCCUGC | CasX 812 |
| | ACGUGUAUUGGGAAGAGUUCCAGAAAGACCCAGUCGGCCUGAUGAGCAGAGUGGC | |
| | UCAGCCUGCCAGCAAGAAGAUCGAUCAGAACAAGCUGAAGCCCGAAAUGGACGAG | |
| | AAGGGGAACCUGACAACCGCCGGCUUUGCCUGUAGCCAGUGCGGCCAGCCCCUGU | |
| | UUGUGUACAAACUGGAACAGGUGAGCGAAAAGGGCAAGGCUUACACGAAUUACUU | |
| | CGGCAGAUGCAACGUGGCCGAGCACGAGAAGCUGAUCCUGCUGGCCCAGCUGAAG | |
| | CCUGAGAAGGAUAGCGAUGAGGCAGUGACAUAUUCCCUGGGCAAGUUCGGACAGC | |
| | GGGCCCUGGAUUUUUAUUCCAUUCAUGUGACCAAGGAAUCCACCCACCCCGUCAA | |
| | GCCUCUUGCCCAAAUUGCCGGCAACAGAUACGCCAGCGGCCCCGUGGGCAAGGCC | |
| | CUGAGCGACGCCUGUAUGGGCACCAUCGCCAGCUUCCUGUCUAAGUACCAGGACA | |
| | UUAUCAUCGAGCACCAGAAGGUGGUGAAGGGCAACCAGAAGAGACUGGAGAGCCU | |
| | GCGCGAGCUGGCCGGCAAGGAAAACCUGGAGUAUCCUAGCGUGACCCUGCCUCCU | |
| | CAGCCUCAUACAAAGGAGGGCGUGGAUGCCUACAACGAAGUGAUCGCCCGGGUGC | |
| | GGAUGUGGGUGAACCUGAAUCUGUGGCAGAAGCUGAAGCUGUCUAGAGACGACGC | |
| | CAAGCCCCUGCUGAGACUGAAGAAGUUCCCCAGCUUCCCUCUGGUGGAGAGACAG | |
| | GCAAAUGAAGUGGACUGGUGGGACAUGGUGUGUAACGUGAAGAAGCUGAUCAAUG | |
| | AGAAGAAGGAGGACGGCAAAGUGUUCUGGCAGAAUCUGGCCGGCUACAAGCGUCA | |
| | GGAGGCCCUGCGGCCCUACCUGAGCAGCGAGGAAGACAGAAAGAAGGGCAAGAAG | |
| | UUCGCCCGGUAUCAGCUGGGGGACCUGCUGCUGCACCUCGAGAAGAAGCACGGCG | |
| | AAGACUGGGGGAAGGUGUACGAUGAGGCCUGGGAGCGGAUCGAUAAGAAGGUGGA | |
| | GGGCCUGAGCAAGCACAUCAAGCUGGAGGAGGAACGGAGAUCUGAGGACGCCCAG | |
| | AGCAAGGCCGCCCUGACCGACUGGCUGAGAGCCAAGGCCAGCUUCGUCAUCGAGG | |
| | GGCUGAAGGAGGCCGACAAGGACGAGUUCUGCCGGUGCGAACUGAAGCUGCAGAA | |
| | GUGGUACGGAGAUCUGAGAGGCAAACCUUUCGCCAUCGAGGCCGAGAACAGCAUC | |
| | CUGGACAUCAGCGGCUUCAGCAAGCAGUACAACUGCGCCUUUAUUUGGCAGAAGG | |
| | ACGGAGUGAAGAAGCUGAACCUGUACCUGAUCAUCAACUAUUUCAAGGGCGGCAA | |
| | GCUGAGAUUCAAGAAGAUCAAGCCUGAAGCCUUCGAGGCCAACAGAUUCUACACC | |
| | GUGAUUAACAAGAAAAGCGGAGAGAUCGUGCCAAUGGAAGUGAACUUCAACUUCG | |
| | ACGACCCUAACCUGAUCAUCCUGCCCCUGGCAUUUGGCAAGCGGCAGGGCAGAGA | |
| | GUUCAUCUGGAACGACCUGCUGUCUCUGGAGACCGGCAGCCUGAAGCUGGCCAAC | |
| | GGCAGAGUGAUCGAGAAGACACUGUACAACAGACGAACCAGACAAGACGAGCCCG | |
| | CCCUGUUUGUGGCCCUGACCUUCGAGAGAAGAGAGGUGCUGGACAGCAGCAAUAU | |
| | CAAGCCUAUGAACCUGAUCGGCGUGGACCGGGGCGAGAACAUCCCUGCCGUGAUC | |
| | GCCCUUACCGACCCCGAGGGAUGCCCUCUGAGCCGGUUUAAAGACAGCCUGGGCA | |
| | ACCCUACCCACAUCCUGAGAAUUGGCGAGUCCUACAAGGAGAAGCAGAGAACCAU | |
| | CCAGGCCAAGAAGGAGGUGGAGCAGCGGCGGGCUGGCGGCUACUCCCGGAAGUAC | |
| | GCCAGCAAGGCCAAGAACCUGGCCGACGACAUGGUUAGAAAUACCGCCAGAGACC | |
| | UCCUGUACUACGCUGUGACCCAGGACGCCAUGCUGAUCUUCGAGAACCUGAGCAG | |
| | AGGCUUCGGCAGACAGGGCAAGAGAACCUUCAUGGCCGAGAGACAGUACACCCGG | |
| | AUGGAGGACUGGCUGACCGCCAAGCUGGCCUACGAGGGCCUGCCCUCUAAGACCU | |
| | ACCUGUCCAAGACCUUGGCACAGUACACCAGCAAGACAUGCUCUAACUGCGGCUU | |
| | CACAAUCACGAGCGCCGACUACGACCGGGUGCUGGAGAAACUGAAGAAGACCGCC | |
| | ACAGGCUGGAUGACCACCAUUAACGGCAAGGAGCUGAAGGUGGAGGGCCAGAUCA | |
| | CCUACUACAACAGGUACAAACGGCAGAACGUGGUGAAGGACCUGAGCGUGGAACU | |
| | GGAUAGACUGAGCGAGGAAAGCGUAAACAAUGACAUCAGCAGCUGGACCAAGGGC | |
| | CGGAGCGGCGAGGCCCUGAGCCUGCUGAAGAAGAGAUUCUCCCACAGACCAGUGC | |
| | AGGAGAAGUUCGUGUGUCUGAACUGCGGCUUCGAGACCCACGCCGACGAGCAAGC | |
| | CGCCCUGAACAUCGCCCGGUCUUGGCUUUUCCUGCGGAGCCAGGAGUACAAGAAG | |
| | UACCAGACAAACAAGACCACAGGCAACACAGACAAGAGAGCCUUCGUCGAGACCU | |
| | GGCAGAGCUUCUACAGAAAGAAGCUGAAGGAGGUGUGGAAGCCUGCCGUGGGAAG | |
| | CCCCGCUGCCAAGAGAGUGAAGCUGGACUAAGCUGCCUUCUGCGGGGCUUGCCUU | |
| | CUGGCCAUGCCCUUCUUCUCUCCCUUGCACCUGUACCUCUUGGUCUUUGAAUAAA | |
| | GCCUGAGUAGGAAGUCUAGAAAAAAAAAAAAAAAAAAAAAAAAAAAAAAAAAAAA | |
| | AAAAAAAAAAAAAAAAAAAAAAAAAAAAAAAAAAAAAAAAAAA | |
| 19834 | AGGAAAmψAAGAGAGAAAAGAAGAGmψAAGAAGAAAmψAmψAAGAGCCACCAmψG | Full-length |
| | GCCCCmψGCmψGCCAAGAGAGmψGAAGCmψGGAmψAGCAGACAGGAGAmψCAAGC | N1- |
| | GGAmψmψAAmψAAAAmψmψCGGAGAAGACmψGGmψGAAGGAmψmψCmψAACACAA | methylpse |
| | AGAAGGCmψGGCAAGACAGGCCCmψAmψGAAGACACmψGCmψGGmψGAGAGmψGA | udouridine |
| | mψGACACCCGACCmψGAGAGAAAGACmψGGAAAACCmψGAGAAAGAAGCCmψGAG | substituted |
| | AAmψAmψCCCCCAGCCCAmψCAGCAACACAAGCCGGGCCAACCmψGAAmψAAGCm | RNA |
| | ψGCmψGACCGACmψACACCGAAAmψGAAGAAGGCCAmψCCmψCGACGmψGmψAmψ | sequence |
| | mψGGGAAGAGmψmψCCAGAAAGACCCAGmψCGGCCmψGAmψGAGCAGAGmψGGCm | of mRNA |
| | ψCAGCCmψGCCAGCAAGAAGAmψCGAmψCAGAACAAGCmψGAAGCCCGAAAmψGG | encoding |
| | ACGAGAAGGGGAACCmψGACAACCGCCGGCmψmψmψGCCmψGmψAGCCAGmψGCG | CasX 491 |
| | GCCAGCCCCmψGmψmψmψGmψGmψACAAACmψGGAACAGGmψGAGCGAAAAGGGC | |
| | AAGGCmψmψACACGAAmψmψACmψmψCGGCAGAmψGCAACGmψGGCCGAGCACGA | |
| | GAAGCmψGAmψCCmψGCmψGGCCCAGCmψGAAGCCmψGAGAAGGAmψAGCGAmψG | |
| | AGGCAGmψGACAmψAmψmψCCCmψGGGCAAGmψmψCGGACAGCGGGCCCmψGGAm | |
| | ψmψmψmψmψAmψmψCCAmψmψCAmψGmψGACCAAGGAAmψCCACCCACCCCGmψC | |
| | AAGCCmψCmψmψGCCCAAAmψmψGCCGGCAACAGAmψACGCCAGCGGCCCCGmψG | |
| | GGCAAGGCCCmψGAGCGACGCCmψGmψAmψGGGCACCAmψCGCCAGCmψmψCCmψ | |
| | GmψCmψAAGmψACCAGGACAmψmψAmψCAmψCGAGCACCAGAAGGmψGGmψGAAG | |
| | GGCAACCAGAAGAGACmψGGAGAGCCmψGCGCGAGCmψGGCCGGCAAGGAAAACC | |
| | mψGGAGmψAmψCCmψAGCGmψGACCCmψGCCmψCCmψCAGCCmψCAmψACAAAGG | |
| | AGGGCGmψGGAmψGCCmψACAACGAAGmψGAmψCGCCCGGGmψGCGGAmψGmψGG | |
| | GmψGAACCmψGAAmψCmψGmψGGCAGAAGCmψGAAGCmψGmψCmψAGAGACGACG | |
| | CCAAGCCCCmψGCmψGAGACmψGAAGGGCmψmψCCCCAGCmψmψCCCmψCmψGGm | |
| | ψGGAGAGACAGGCAAAmψGAAGmψGGACmψGGmψGGGACAmψGGmψGmψGmψAAC | |
| | GmψGAAGAAGCmψGAmψCAAmψGAGAAGAAGGAGGACGGCAAAGmψGmψmψCmψG | |
| | GCAGAAmψCmψGGCCGGCmψACAAGCGmψCAGGAGGCCCmψGCGGCCCmψACCmψ | |
| | GAGCAGCGAGGAAGACAGAAAGAAGGGCAAGAAGmψmψCGCCCGGmψAmψCAGCm | |
| | ψGGGGGACCmψGCmψGCmψGCACCmψCGAGAAGAAGCACGGCGAAGACmψGGGGG | |
| | AAGGmψGmψACGAmψGAGGCCmψGGGAGCGGAmψCGAmψAAGAAGGmψGGAGGGC | |
| | CmψGAGCAAGCACAmψCAAGCmψGGAGGAGGAACGGAGAmψCmψGAGGACGCCCA | |
| | GAGCAAGGCCGCCCmψGACCGACmψGGCmψGAGAGCCAAGGCCAGCmψmψCGmψC | |
| | AmψCGAGGGGCmψGAAGGAGGCCGACAAGGACGAGmψmψCmψGCCGGmψGCGAAC | |
| | mψGAAGCmψGCAGAAGmψGGmψACGGAGAmψCmψGAGAGGCAAACCmψmψmψCGA | |
| | CAmψCGAGGCCGAGAACAGCAmψCCmψGGACAmψCAGCGGCmψmψCAGCAAGCAG | |
| | mψACAACmψGCGCCmψmψmψAmψmψmψGGCAGAAGGACGGAGmψGAAGAAGCmψG | |
| | AACCmψGmψACCmψGAmψCAmψCAACmψAmψmψmψCAAGGGCGGCAAGCmψGAGC | |
| | mψmψCAAGAAGAmψCAAGCCmψGAAGCCmψmψCGAGGCCAACAGAmψmψCmψACA | |
| | CCGmψGAmψmψAACAAGAAAAGCGGAGAGAmψCGmψGCCAAmψGGAAGmψGAACm | |
| | ψmψCAACmψmψCGACGACCCmψAACCmψGAmψCAmψCCmψGCCCCmψGGCAmψmψ | |
| | mψGGCAAGCGGCAGGGCAGAGAGmψmψCAmψCmψGGAACGACCmψGCmψGmψCmψ | |
| | CmψGGAGACCGGCAGCCmψGAAGCmψGGCCAACGGCAGAGmψGAmψCGAGAAGAC | |
| | ACmψGmψACAACAGACGAACCAGACAAGACGAGCCCGCCCmψGmψmψmψGmψGGC | |
| | CCmψGACCmψmψCGAGAGAAGAGAGGmψGCmψGGACAGCAGCAAmψAmψCAAGCC | |
| | mψAmψGAACCmψGAmψCGGCGmψGGACCGGGGCGAGAACAmψCCCmψGCCGmψGA | |
| | AGGAGAAGCAGAGAACCAmψCCAGGCCAAGAAGGAGGmψGGAGCAGCGGCGGGCm | |
| | ψGGCGGCmψACmψCCCGGAAGmψACGCCAGCAAGGCCAAGAACCmψGGCCGACGA | |
| | CCAGGACGCCAmψGCmψGAmψCmψmψCGAGAACCmψGAGCAGAGGCmψmψCGGCA | |
| | GACAGGGCAAGAGAACCmψmψCAmψGGCCGAGAGACAGmψACACCCGGAmψGGAG | |
| | GACmψGGCmψGACCGCCAAGCmψGGCCmψACGAGGGCCmψGmψCmψAAGACCmψA | |
| | CCmψGmψCCAAGACCmψmψGGCACAGmψACACCAGCAAGACAmψGCmψCmψAACm | |
| | ψGCGGCmψmψCACAAmψCACGAGCGCCGACmψACGACCGGGmψGCmψGGAGAAAC | |
| | mψGAAGAAGACCGCCACAGGCmψGGAmψGACCACCAmψmψAACGGCAAGGAGCmψ | |
| | GAAGGmψGGAGGGCCAGAmψCACCmψACmψACAACAGGmψACAAACGGCAGAACG | |
| | mψGGmψGAAGGACCmψGAGCGmψGGAACmψGGAmψAGACmψGAGCGAGGAAAGCG | |
| | mψAAACAAmψGACAmψCAGCAGCmψGGACCAAGGGCCGGAGCGGCGAGGCCCmψG | |
| | AGCCmψGCmψGAAGAAGAGAmψmψCmψCCCACAGACCAGmψGCAGGAGAAGmψmψ | |
| | CGmψGmψGmψCmψGAACmψGCGGCmψmψCGAGACCCACGCCGACGAGCAAGCCGC | |
| | ACAAGAAGmψACCAGACAAACAAGACCACAGGCAACACAGACAAGAGAGCCmψmψ | |
| | CGmψCGAGACCmψGGCAGAGCmψmψCmψACAGAAAGAAGCmψGAAGGAGGmψGmψ | |
| | GGAAGCCmψGCCGmψGGGAAGCCCCGCmψGCCAAGAGAGmψGAAGCmψGGACmψA | |
| | CGAAGCCmψGCCGmψGGGAAGCCCCGCmψGCCAAGAGAGmψGAAGCmψGGACmψA | |
| | CmψGAGmψAGGAAGmψCmψAGAAAAAAAAAAAAAAAAAAAAAAAAAAAAAAAAAA | |
| | AAAAAAAAAAAAAAAAAAAAAAAAAAAAAAAAAAAAAAAAAAAAA | |
| 19835 | AGGAAAmψAAGAGAGAAAAGAAGAGmψAAGAAGAAAmψAmψAAGAGCCACCAmψG | Full-length |
| | GCCCCmψGCmψGCCAAGAGAGmψGAAGCmψGGAmψAGCAGACAGGAGAmψCAAGC | N1- |
| | GGAmψmψAAmψAAAAmψmψCGGAGAAGACmψGGmψGAAGGAmψmψCmψAACACAA | methylpse |
| | AGAAGGCmψGGCAAGACAGGCCCmψAmψGAAGACACmψGCmψGGmψGAGAGmψGA | udouridine |
| | mψGACACCCGACCmψGAGAGAAAGACmψGGAAAACCmψGAGAAAGAAGCCmψGAG | substituted |
| | AAmψAmψCCCCCAGCCCAmψCAGCAACACAAGCCGGGCCAACCmψGAAmψAAGCm | RNA |
| | ψGCmψGACCGACmψACACCGAAAmψGAAGAAGGCCAmψCCmψCGACGmψGmψAmψ | sequence |
| | mψGGGAAGAGmψmψCCAGAAAGACCCAGmψCGGCCmψGAmψGAGCAGAGmψGGCm | of mRNA |
| | ψCAGCCmψGCCAGCAAGAAGAmψCGAmψCAGAACAAGCmψGAAGCCCGAAAmψGG | encoding |
| | ACGAGAAGGGGAACCmψGACAACCGCCGGCmψmψmψGCCmψGmψAGCCAGmψGCG | CasX 515 |
| | GCCAGCCCCmψGmψmψmψGmψGmψACAAACmψGGAACAGGmψGAGCGAAAAGGGC | |
| | AAGGCmψmψACACGAAmψmψACmψmψCGGCAGAmψGCAACGmψGGCCGAGCACGA | |
| | GAAGCmψGAmψCCmψGCmψGGCCCAGCmψGAAGCCmψGAGAAGGAmψAGCGAmψG | |
| | AGGCAGmψGACAmψAmψmψCCCmψGGGCAAGmψmψCGGACAGCGGGCCCmψGGAm | |
| | ψmψmψmψmψAmψmψCCAmψmψCAmψGmψGACCAAGGAAmψCCACCCACCCCGmψC | |
| | AAGCCmψCmψmψGCCCAAAmψmψGCCGGCAACAGAmψACGCCAGCGGCCCCGmψG | |
| | GGCAAGGCCCmψGAGCGACGCCmψGmψAmψGGGCACCAmψCGCCAGCmψmψCCmψ | |
| | GmψCmψAAGmψACCAGGACAmψmψAmψCAmψCGAGCACCAGAAGGmψGGmψGAAG | |
| | GGCAACCAGAAGAGACmψGGAGAGCCmψGCGCGAGCmψGGCCGGCAAGGAAAACC | |
| | mψGGAGmψAmψCCmψAGCGmψGACCCmψGCCmψCCmψCAGCCmψCAmψACAAAGG | |
| | AGGGCGmψGGAmψGCCmψACAACGAAGmψGAmψCGCCCGGGmψGCGGAmψGmψGG | |
| | GmψGAACCmψGAAmψCmψGmψGGCAGAAGCmψGAAGCmψGmψCmψAGAGACGACG | |
| | CCAAGCCCCmψGCmψGAGACmψGAAGGGCmψmψCCCCAGCmψmψCCCmψCmψGGm | |
| | ψGGAGAGACAGGCAAAmψGAAGmψGGACmψGGmψGGGACAmψGGmψGmψGmψAAC | |
| | GmψGAAGAAGCmψGAmψCAAmψGAGAAGAAGGAGGACGGCAAAGmψGmψmψCmψG | |
| | GCAGAAmψCmψGGCCGGCmψACAAGCGmψCAGGAGGCCCmψGCGGCCCmψACCmψ | |
| | GAGCAGCGAGGAAGACAGAAAGAAGGGCAAGAAGmψmψCGCCCGGmψAmψCAGCm | |
| | ψGGGGGACCmψGCmψGCmψGCACCmψCGAGAAGAAGCACGGCGAAGACmψGGGGG | |
| | AAGGmψGmψACGAmψGAGGCCmψGGGAGCGGAmψCGAmψAAGAAGGmψGGAGGGC | |
| | CmψGAGCAAGCACAmψCAAGCmψGGAGGAGGAACGGAGAmψCmψGAGGACGCCCA | |
| | GAGCAAGGCCGCCCmψGACCGACmψGGCmψGAGAGCCAAGGCCAGCmψmψCGmψC | |
| | AmψCGAGGGGCmψGAAGGAGGCCGACAAGGACGAGmψmψCmψGCCGGmψGCGAAC | |
| | mψGAAGCmψGCAGAAGmψGGmψACGGAGAmψCmψGAGAGGCAAACCmψmψmψCGA | |
| | CAmψCGAGGCCGAGAACAGCAmψCCmψGGACAmψCAGCGGCmψmψCAGCAAGCAG | |
| | mψACAACmψGCGCCmψmψmψAmψmψmψGGCAGAAGGACGGAGmψGAAGAAGCmψG | |
| | AACCmψGmψACCmψGAmψCAmψCAACmψAmψmψmψCAAGGGCGGCAAGCmψGAGC | |
| | mψmψCAAGAAGAmψCAAGCCmψGAAGCCmψmψCGAGGCCAACAGAmψmψCmψACA | |
| | CCGmψGAmψmψAACAAGAAAAGCGGAGAGAmψCGmψGCCAAmψGGAAGmψGAACm | |
| | ψmψCAACmψmψCGACGACCCmψAACCmψGAmψCAmψCCmψGCCCCmψGGCAmψmψ | |
| | mψGGCAAGCGGCAGGGCAGAGAGmψmψCAmψCmψGGAACGACCmψGCmψGmψCmψ | |
| | CmψGGAGACCGGCAGCCmψGAAGCmψGGCCAACGGCAGAGmψGAmψCGAGAAGAC | |
| | ACmψGmψACAACAGACGAACCAGACAAGACGAGCCCGCCCmψGmψmψmψGmψGGC | |
| | CCmψGACCmψmψCGAGAGAAGAGAGGmψGCmψGGACAGCAGCAAmψAmψCAAGCC | |
| | mψAmψGAACCmψGAmψCGGCGmψGGACCGGGGCGAGAACAmψCCCmψGCCGmψGA | |
| | mψCGCCCmψmψACCGACCCCGAGGGAmψGCCCmψCmψGAGCCGGmψmψmψAAAGA | |
| | CAGCCmψGGGCAACCCmψACCCACAmψCCmψGAGAAmψmψGGCGAGmψCCmψACA | |
| | AGGAGAAGCAGAGAACCAmψCCAGGCCAAGAAGGAGGmψGGAGCAGCGGCGGGCm | |
| | ψGGCGGCmψACmψCCCGGAAGmψACGCCAGCAAGGCCAAGAACCmψGGCCGACGA | |
| | CAmψGGmψmψAGAAAmψACCGCCAGAGACCmψCCmψGmψACmψACGCmψGmψGAC | |
| | CCAGGACGCCAmψGCmψGAmψCmψmψCGAGAACCmψGAGCAGAGGCmψmψCGGCA | |
| | GACAGGGCAAGAGAACCmψmψCAmψGGCCGAGAGACAGmψACACCCGGAmψGGAG | |
| | GACmψGGCmψGACCGCCAAGCmψGGCCmψACGAGGGCCmψGCCCmψCmψAAGACC | |
| | mψACCmψGmψCCAAGACCmψmψGGCACAGmψACACCAGCAAGACAmψGCmψCmψA | |
| | ACmψGCGGCmψmψCACAAmψCACGAGCGCCGACmψACGACCGGGmψGCmψGGAGA | |
| | AACmψGAAGAAGACCGCCACAGGCmψGGAmψGACCACCAmψmψAACGGCAAGGAG | |
| | CmψGAAGGmψGGAGGGCCAGAmψCACCmψACmψACAACAGGmψACAAACGGCAGA | |
| | ACGmψGGmψGAAGGACCmψGAGCGmψGGAACmψGGAmψAGACmψGAGCGAGGAAA | |
| | GCGmψAAACAAmψGACAmψCAGCAGCmψGGACCAAGGGCCGGAGCGGCGAGGCCC | |
| | mψGAGCCmψGCmψGAAGAAGAGAmψmψCmψCCCACAGACCAGmψGCAGGAGAAGm | |
| | ψmψCGmψGmψGmψCmψGAACmψGCGGCmψmψCGAGACCCACGCCGACGAGCAAGC | |
| | CGCCCmψGAACAmψCGCCCGGmψCmψmψGGCmψmψmψmψCCmψGCGGAGCCAGGA | |
| | GmψACAAGAAGmψACCAGACAAACAAGACCACAGGCAACACAGACAAGAGAGCCm | |
| | ψmψCGmψCGAGACCmψGGCAGAGCmψmψCmψACAGAAAGAAGCmψGAAGGAGGmψ | |
| | GmψGGAAGCCmψGCCGmψGGGAAGCCCCGCmψGCCAAGAGAGmψGAAGCmψGGAC | |
| | mψAAGCmψGCCmψmψCmψGCGGGGCmψmψGCCmψmψCmψGGCCAmψGCCCmψmψC | |
| | mψmψCmψCmψCCCmψmψGCACCmψGmψACCmψCmψmψGGmψCmψmψmψGAAmψAA | |
| | AGCCmψGAGmψAGGAAGmψCmψAGAAAAAAAAAAAAAAAAAAAAAAAAAAAAAAA | |
| | AAAAAAAAAAAAAAAAAAAAAAAAAAAAAAAAAAAAAAAAAAAAAAAA | |
| 19836 | AGGAAAmψAAGAGAGAAAAGAAGAGmψAAGAAGAAAmψAmψAAGAGCCACCAmψG | Full-length |
| | GCCCCmψGCmψGCCAAGAGAGmψGAAGCmψGGAmψAGCAGACAGGAGAmψCAAGC | N1- |
| | GGAmψmψAAmψAAAAmψmψCGGAGAAGACmψGGmψGAAGGAmψmψCmψAACACAA | methylpse |
| | AGAAGGCmψGGCAAGACACGGGGCCCmψAmψGAAGACACmψGCmψGGmψGAGAGm | udouridine |
| | ψGAmψGACACCCGACCmψGAGAGAAAGACmψGGAAAACCmψGAGAAAGAAGCCmψ | (mu) |
| | GAGAAmψAmψCCCCCAGCCCAmψCAGCAACACAAGCCGGGCCAACCmψGAAmψAA | substituted |
| | GCmψGCmψGACCGACmψACACCGAAAmψGAAGAAGGCCAmψCCmψGCACGmψGmψ | RNA |
| | AmψmψGGGAAGAGmψmψCCAGAAAGACCCAGmψCGGCCmψGAmψGAGCAGAGmψG | sequence |
| | GCmψCAGCCmψGCCAGCAAGAAGAmψCGAmψCAGAACAAGCmψGAAGCCCGAAAm | of mRNA |
| | ψGGACGAGAAGGGGAACCmψGACAACCGCCGGCmψmψmψGCCmψGmψAGCCAGmψ | encoding |
| | GCGGCCAGCCCCmψGmψmψmψGmψGmψACAAACmψGGAACAGGmψGAGCGAAAAG | CasX 676 |
| | GGCAAGGCmψmψACACGAAmψmψACmψmψCGGCAGAmψGCAACGmψGGCCGAGCA | |
| | CGAGAAGCmψGAmψCAAGCmψGGCCCAGCmψGAAGCCmψGAGAAGGAmψAGCGAm | |
| | ψGAGGCAGmψGACAmψAmψmψCCCmψGGGCAAGmψmψCGGACAGCGGGCCCmψGG | |
| | AmψmψmψmψmψAmψmψCCAmψmψCAmψGmψGACCAAGGAAmψCCACCCACCCCGm | |
| | ψCAAGCCmψCmψmψGCCCAAAmψmψGCCGGCAACAGAmψACGCCmψCCAGCCCCG | |
| | mψGGGCAAGGCCCmψGAGCGACGCCmψGmψAmψGGGCACCAmψCGCCAGCmψmψC | |
| | CmψGmψCmψAAGmψACCAGGACAmψmψAmψCAmψCGAGCACCAGAAGGmψGGmψG | |
| | AAGGGCAACCAGAAGAGACmψGGAGAGCCmψGCGCGAGCmψGGCCGGCAAGGAAA | |
| | ACCmψGGAGmψAmψCCmψAGCGmψGACCCmψGCCmψCCmψCAGCCmψCAmψACAA | |
| | AGGAGGGCGmψGGAmψGCCmψACAACGAAGmψGAmψCGCCCGGGmψGCGGAmψGm | |
| | ψGGGmψGAACCmψGAAmψCmψGmψGGCAGAAGCmψGAAGCmψGmψCmψAGAGACG | |
| | ACGCCAAGCCCCmψGCmψGAGACmψGAAGGGCmψmψCCCCAGCmψmψCCCmψCmψ | |
| | GGmψGGAGAGACAGGCAAAmψGAAGmψGGACmψGGmψGGGACAmψGGmψGmψGmψ | |
| | AACGmψGAAGAAGCmψGAmψCAAmψGAGAAGAAGGAGGACGGCAAAGmψGmψmψC | |
| | mψGGCAGAAmψCmψGGCCGGCmψACAAGCGmψCAGGAGGCCCmψGCGGCCCmψAC | |
| | CmψGAGCAGCGAGGAAGACAGAAAGAAGGGCAAGAAGmψmψCGCCCGGmψAmψCA | |
| | GCmψGGGGGACCmψGCmψGCmψGCACCmψCGAGAAGAAGCACGGCGAAGACmψGG | |
| | GGGAAGGmψGmψACGAmψGAGGCCmψGGGAGCGGAmψCGAmψAAGAAGGmψGGAG | |
| | GGCCmψGAGCAAGCACAmψCAAGCmψGGAGGAGGAACGGAGAmψCmψGAGGACGC | |
| | CCAGAGCAAGGCCGCCCmψGACCGACmψGGCmψGAGAGCCAAGGCCAGCmψmψCG | |
| | mψCAmψCGAGGGGCmψGAAGGAGGCCGACAAGGACGAGmψmψCmψGCCGGmψGCG | |
| | AACmψGAAGCmψGCAGAAGmψGGmψACGGAGAmψCmψGAGAGGCAAACCmψmψmψ | |
| | CGCCAmψCGAGGCCGAGAACAGCAmψCCmψGGACAmψCAGCGGCmψmψCAGCAAG | |
| | CAGmψACAACmψGCGCCmψmψmψAmψmψmψGGCAGAAGGACGGAGmψGAAGAAGC | |
| | mψGAACCmψGmψACCmψGAmψCAmψCAACmψAmψmψmψCAAGGGCGGCAAGCmψG | |
| | AGAmψmψCAAGAAGAmψCAAGCCmψGAAGCCmψmψCGAGGCCAACAGAmψmψCmψ | |
| | ACACCGmψGAmψmψAACAAGAAAAGCGGAGAGAmψCGmψGCCAAmψGGAAGmψGA | |
| | ACmψmψCAACmψmψCGACGACCCmψAACCmψGAmψCAmψCCmψGCCCCmψGGCAm | |
| | ψmψmψGGCAAGCGGCAGGGCAGAGAGmψmψCAmψCmψGGAACGACCmψGCmψGmψ | |
| | CmψCmψGGAGACCGGCAGCCmψGAAGCmψGGCCAACGGCAGAGmψGAmψCGAGAA | |
| | GACACmψGmψACAACAGACGAACCAGACAAGACGAGCCCGCCCmψGmψmψmψGmψ | |
| | GGCCCmψGACCmψmψCGAGAGAAGAGAGGmψGCmψGGACAGCAGCAAmψAmψCAA | |
| | GCCmψAmψGAACCmψGAmψCGGCGmψGGACCGGGGCGAGAACAmψCCCmψGCCGm | |
| | ψGAmψCGCCCmψmψACCGACCCCGAGGGAmψGCCCmψCmψGAGCCGGmψmψmψAA | |
| | AGACAGCCmψGGGCAACCCmψACCCACAmψCCmψGAGAAmψmψGGCGAGmψCCmψ | |
| | ACAAGGAGAAGCAGAGAACCAmψCCAGGCCAAGAAGGAGGmψGGAGCAGCGGCGG | |
| | GCmψGGCGGCmψACmψCCCGGAAGmψACGCCAGCAAGGCCAAGAACCmψGGCCGA | |
| | CGACAmψGGmψmψAGAAAmψACCGCCAGAGACCmψCCmψGmψACmψACGCmψGmψ | |
| | GACCCAGGACGCCAmψGCmψGAmψCmψmψCGAGAACCmψGAGCAGAGGCmψmψCG | |
| | GCAGACAGGGCAAGAGAACCmψmψCAmψGGCCGAGAGACAGmψACACCCGGAmψG | |
| | GAGGACmψGGCmψGACCGCCAAGCmψGGCCmψACGAGGGCCmψGCCCmψCmψAAG | |
| | ACCmψACCmψGmψCCAAGACCmψmψGGCACAGmψACACCAGCAAGACAmψGCmψC | |
| | mψAACmψGCGGCmψmψCACAAmψCACGAGCGCCGACmψACGACCGGGmψGCmψGG | |
| | AGAAACmψGAAGAAGACCGCCACAGGCmψGGAmψGACCACCAmψmψAACGGCAAG | |
| | GAGCmψGAAGGmψGGAGGGCCAGAmψCACCmψACmψACAACAGGmψACAAACGGC | |
| | AGAACGmψGGmψGAAGGACCmψGAGCGmψGGAACmψGGAmψAGACmψGAGCGAGG | |
| | AAAGCGmψAAACAAmψGACAmψCAGCAGCmψGGACCAAGGGCCGGAGCGGCGAGG | |
| | CCCmψGAGCCmψGCmψGAAGAAGAGAmψmψCmψCCCACAGACCAGmψGCAGGAGA | |
| | AGmψmψCGmψGmψGmψCmψGAACmψGCGGCmψmψCGAGACCCACGCCGACGAGCA | |
| | AGCCGCCCmψGAACAmψCGCCCGGmψCmψmψGGCmψmψmψmψCCmψGCGGAGCCA | |
| | GGAGmψACAAGAAGmψACCAGACAAACAAGACCACAGGCAACACAGACAAGAGAG | |
| | CCmψmψCGmψCGAGACCmψGGCAGAGCmψmψCmψACAGAAAGAAGCmψGAAGGAG | |
| | GmψGmψGGAAGCCmψGCCGmψGGGAAGCCCCGCmψGCCAAGAGAGmψGAAGCmψG | |
| | GACmψAAGCmψGCCmψmψCmψGCGGGGCmψmψGCCmψmψCmψGGCCAmψGCCCmψ | |
| | mψCmψmψCmψCmψCCCmψmψGCACCmψGmψACCmψCmψmψGGmψCmψmψmψGAAm | |
| | ψAAAGCCmψGAGmψAGGAAGmψCmψAGAAAAAAAAAAAAAAAAAAAAAAAAAAAA | |
| | AAAAAAAAAAAAAAAAAAAAAAAAAAAAAAAAAAAAAAAAAAAAAAAAAAA | |
| 19837 | AGGAAAmψAAGAGAGAAAAGAAGAGmψAAGAAGAAAmψAmψAAGAGCCACCAmψG | Full-length |
| | GCCCCmψGCmψGCCAAGAGAGmψGAAGCmψGGAmψAGCAGACAGGAGAmψCAAGC | N1- |
| | GGAmψmψAAmψAAAAmψmψCGGAGAAGACmψGGmψGAAGGAmψmψCmψAACACAA | methylpse |
| | AGAAGGCmψGGCAAGACAGGCCCmψAmψGAAGACACmψGCmψGGmψGAGAGmψGA | udouridine |
| | mψGACACCCGACCmψGAGAGAAAGACmψGGAAAACCmψGAGAAAGAAGCCmψGAG | substituted |
| | AAmψAmψCCCCCAGCCCAmψCAGCAACACAAGCCGGGCCAACCmψGAAmψAAGCm | RNA |
| | mψGGGAAGAGmψmψCCAGAAAGACCCAGmψCGGCCmψGAmψGAGCAGAGmψGGCm | sequence |
| | ψCAGCCmψGCCAGCAAGAAGAmψCGAmψCAGAACAAGCmψGAAGCCCGAAAmψGG | of mRNA |
| | ACGAGAAGGGGAACCmψGACAACCGCCGGCmψmψmψGCCmψGmψAGCCAGmψGCG | encoding |
| | GCCAGCCCCmψGmψmψmψGmψGmψACAAACmψGGAACAGGmψGAGCGAAAAGGGC | CasX 812 |
| | AAGGCmψmψACACGAAmψmψACmψmψCGGCAGAmψGCAACGmψGGCCGAGCACGA | |
| | GAAGCmψGAmψCCmψGCmψGGCCCAGCmψGAAGCCmψGAGAAGGAmψAGCGAmψG | |
| | AGGCAGmψGACAmψAmψmψCCCmψGGGCAAGmψmψCGGACAGCGGGCCCmψGGAm | |
| | ψmψmψmψmψAmψmψCCAmψmψCAmψGmψGACCAAGGAAmψCCACCCACCCCGmψC | |
| | AAGCCmψCmψmψGCCCAAAmψmψGCCGGCAACAGAmψACGCCAGCGGCCCCGmψG | |
| | GGCAAGGCCCmψGAGCGACGCCmψGmψAmψGGGCACCAmψCGCCAGCmψmψCCmψ | |
| | GmψCmψAAGmψACCAGGACAmψmψAmψCAmψCGAGCACCAGAAGGmψGGmψGAAG | |
| | GGCAACCAGAAGAGACmψGGAGAGCCmψGCGCGAGCmψGGCCGGCAAGGAAAACC | |
| | mψGGAGmψAmψCCmψAGCGmψGACCCmψGCCmψCCmψCAGCCmψCAmψACAAAGG | |
| | AGGGCGmψGGAmψGCCmψACAACGAAGmψGAmψCGCCCGGGmψGCGGAmψGmψGG | |
| | GmψGAACCmψGAAmψCmψGmψGGCAGAAGCmψGAAGCmψGmψCmψAGAGACGACG | |
| | CCAAGCCCCmψGCmψGAGACmψGAAGGGCmψmψCCCCAGCmψmψCCCmψCmψGGm | |
| | ψGGAGAGACAGGCAAAmψGAAGmψGGACmψGGmψGGGACAmψGGmψGmψGmψAAC | |
| | GmψGAAGAAGCmψGAmψCAAmψGAGAAGAAGGAGGACGGCAAAGmψGmψmψCmψG | |
| | GCAGAAmψCmψGGCCGGCmψACAAGCGmψCAGGAGGCCCmψGCGGCCCmψACCmψ | |
| | GAGCAGCGAGGAAGACAGAAAGAAGGGCAAGAAGmψmψCGCCCGGmψAmψCAGCm | |
| | ψGGGGGACCmψGCmψGCmψGCACCmψCGAGAAGAAGCACGGCGAAGACmψGGGGG | |
| | AAGGmψGmψACGAmψGAGGCCmψGGGAGCGGAmψCGAmψAAGAAGGmψGGAGGGC | |
| | CmψGAGCAAGCACAmψCAAGCmψGGAGGAGGAACGGAGAmψCmψGAGGACGCCCA | |
| | GAGCAAGGCCGCCCmψGACCGACmψGGCmψGAGAGCCAAGGCCAGCmψmψCGmψC | |
| | AmψCGAGGGGCmψGAAGGAGGCCGACAAGGACGAGmψmψCmψGCCGGmψGCGAAC | |
| | mψGAAGCmψGCAGAAGmψGGmψACGGAGAmψCmψGAGAGGCAAACCmψmψmψCGA | |
| | CAmψCGAGGCCGAGAACAGCAmψCCmψGGACAmψCAGCGGCmψmψCAGCAAGCAG | |
| | mψACAACmψGCGCCmψmψmψAmψmψmψGGCAGAAGGACGGAGmψGAAGAAGCmψG | |
| | AACCmψGmψACCmψGAmψCAmψCAACmψAmψmψmψCAAGGGCGGCAAGCmψGAGC | |
| | mψmψCAAGAAGAmψCAAGCCmψGAAGCCmψmψCGAGGCCAACAGAmψmψCmψACA | |
| | CCGmψGAmψmψAACAAGAAAAGCGGAGAGAmψCGmψGCCAAmψGGAAGmψGAACm | |
| | ψmψCAACmψmψCGACGACCCmψAACCmψGAmψCAmψCCmψGCCCCmψGGCAmψmψ | |
| | mψGGCAAGCGGCAGGGCAGAGAGmψmψCAmψCmψGGAACGACCmψGCmψGmψCmψ | |
| | CmψGGAGACCGGCAGCCmψGAAGCmψGGCCAACGGCAGAGmψGAmψCGAGAAGAC | |
| | ACmψGmψACAACAGACGAACCAGACAAGACGAGCCCGCCCmψGmψmψmψGmψGGC | |
| | CCmψGACCmψmψCGAGAGAAGAGAGGmψGCmψGGACAGCAGCAAmψAmψCAAGCC | |
| | mψAmψGAACCmψGAmψCGGCGmψGGACCGGGGCGAGAACAmψCCCmψGCCGmψGA | |
| | mψCGCCCmψmψACCGACCCCGAGGGAmψGCCCmψCmψGAGCCGGmψmψmψAAAGA | |
| | CAGCCmψGGGCAACCCmψACCCACAmψCCmψGAGAAmψmψGGCGAGmψCCmψACA | |
| | AGGAGAAGCAGAGAACCAmψCCAGGCCAAGAAGGAGGmψGGAGCAGCGGCGGGCm | |
| | ψGGCGGCmψACmψCCCGGAAGmψACGCCAGCAAGGCCAAGAACCmψGGCCGACGA | |
| | CAmψGGmψmψAGAAAmψACCGCCAGAGACCmψCCmψGmψACmψACGCmψGmψGAC | |
| | CCAGGACGCCAmψGCmψGAmψCmψmψCGAGAACCmψGAGCAGAGGCmψmψCGGCA | |
| | GACAGGGCAAGAGAACCmψmψCAmψGGCCGAGAGACAGmψACACCCGGAmψGGAG | |
| | GACmψGGCmψGACCGCCAAGCmψGGCCmψACGAGGGCCmψGCCCmψCmψAAGACC | |
| | mψACCmψGmψCCAAGACCmψmψGGCACAGmψACACCAGCAAGACAmψGCmψCmψA | |
| | ACmψGCGGCmψmψCACAAmψCACGAGCGCCGACmψACGACCGGGmψGCmψGGAGA | |
| | AACmψGAAGAAGACCGCCACAGGCmψGGAmψGACCACCAmψmψAACGGCAAGGAG | |
| | CmψGAAGGmψGGAGGGCCAGAmψCACCmψACmψACAACAGGmψACAAACGGCAGA | |
| | ACGmψGGmψGAAGGACCmψGAGCGmψGGAACmψGGAmψAGACmψGAGCGAGGAAA | |
| | GCGmψAAACAAmψGACAmψCAGCAGCmψGGACCAAGGGCCGGAGCGGCGAGGCCC | |
| | mψGAGCCmψGCmψGAAGAAGAGAmψmψCmψCCCACAGACCAGmψGCAGGAGAAGm | |
| | ψmψCGmψGmψGmψCmψGAACmψGCGGCmψmψCGAGACCCACGCCGACGAGCAAGC | |
| | CGCCCmψGAACAmψCGCCCGGmψCmψmψGGCmψmψmψmψCCmψGCGGAGCCAGGA | |
| | GmψACAAGAAGmψACCAGACAAACAAGACCACAGGCAACACAGACAAGAGAGCCm | |
| | ψmψCGmψCGAGACCmψGGCAGAGCmψmψCmψACAGAAAGAAGCmψGAAGGAGGmψ | |
| | GmψGGAAGCCmψGCCGmψGGGAAGCCCCGCmψGCCAAGAGAGmψGAAGCmψGGAC | |
| | mψAAGCmψGCCmψmψCmψGCGGGGCmψmψGCCmψmψCmψGGCCAmψGCCCmψmψC | |
| | mψmψCmψCmψCCCmψmψGCACCmψGmψACCmψCmψmψGGmψCmψmψmψGAAmψAA | |
| | AGCCmψGAGmψAGGAAGmψCmψAGAAAAAAAAAAAAAAAAAAAAAAAAAAAAAAA | |
| | AAAAAAAAAAAAAAAAAAAAAAAAAAAAAAAAAAAAAAAAAAAAAAAA | |
| 19838 | AGGAAATAAGAGAGAAAAGAAGAGTAAGAAGAAATATAAGAGCCACCATGGCCCC | Full-length |
| | TGCTGCCAAGAGAGTGAAGCTGGATAGCAGACAGGAGATCAAGCGGATTAATAAA | DNA |
| | ATTCGGAGAAGACTGGTGAAGGATTCTAACACAAAGAAGGCTGGCAAGACAGGCC | sequence |
| | CTATGAAGACACTGCTGGTGAGAGTGATGACACCCGACCTGAGAGAAAGACTGGA | of mRNA |
| | AAACCTGAGAAAGAAGCCTGAGAATATCCCCCAGCCCATCAGCAACACAAGCCGG | encoding |
| | GCCAACCTGAATAAGCTGCTGACCGACTACACCGAAATGAAGAAGGCCATCCTGC | CasX 491 |
| | ACGTGTATTGGGAAGAGTTCCAGAAAGACCCAGTCGGCCTGATGAGCAGAGTGGC | |
| | TCAGCCTGCCAGCAAGAAGATCGATCAGAACAAGCTGAAGCCCGAAATGGACGAG | |
| | AAGGGGAACCTGACAACCGCCGGCTTTGCCTGTAGCCAGTGCGGCCAGCCCCTGT | |
| | TTGTGTACAAACTGGAACAGGTGAGCGAAAAGGGCAAGGCTTACACGAATTACTT | |
| | CGGCAGATGCAACGTGGCCGAGCACGAGAAGCTGATCCTGCTGGCCCAGCTGAAG | |
| | CCTGAGAAGGATAGCGATGAGGCAGTGACATATTCCCTGGGCAAGTTCGGACAGC | |
| | GGGCCCTGGATTTTTATTCCATTCATGTGACCAAGGAATCCACCCACCCCGTCAA | |
| | GCCTCTTGCCCAAATTGCCGGCAACAGATACGCCAGCGGCCCCGTGGGCAAGGCC | |
| | CTGAGCGACGCCTGTATGGGCACCATCGCCAGCTTCCTGTCTAAGTACCAGGACA | |
| | TTATCATCGAGCACCAGAAGGTGGTGAAGGGCAACCAGAAGAGACTGGAGAGCCT | |
| | GCGCGAGCTGGCCGGCAAGGAAAACCTGGAGTATCCTAGCGTGACCCTGCCTCCT | |
| | CAGCCTCATACAAAGGAGGGCGTGGATGCCTACAACGAAGTGATCGCCCGGGTGC | |
| | GGATGTGGGTGAACCTGAATCTGTGGCAGAAGCTGAAGCTGTCTAGAGACGACGC | |
| | CAAGCCCCTGCTGAGACTGAAGGGCTTCCCCAGCTTCCCTCTGGTGGAGAGACAG | |
| | GCAAATGAAGTGGACTGGTGGGACATGGTGTGTAACGTGAAGAAGCTGATCAATG | |
| | AGAAGAAGGAGGACGGCAAAGTGTTCTGGCAGAATCTGGCCGGCTACAAGCGTCA | |
| | GGAGGCCCTGCGGCCCTACCTGAGCAGCGAGGAAGACAGAAAGAAGGGCAAGAAG | |
| | TTCGCCCGGTATCAGCTGGGGGACCTGCTGCTGCACCTCGAGAAGAAGCACGGCG | |
| | AAGACTGGGGGAAGGTGTACGATGAGGCCTGGGAGCGGATCGATAAGAAGGTGGA | |
| | GGGCCTGAGCAAGCACATCAAGCTGGAGGAGGAACGGAGATCTGAGGACGCCCAG | |
| | AGCAAGGCCGCCCTGACCGACTGGCTGAGAGCCAAGGCCAGCTTCGTCATCGAGG | |
| | GGCTGAAGGAGGCCGACAAGGACGAGTTCTGCCGGTGCGAACTGAAGCTGCAGAA | |
| | GTGGTACGGAGATCTGAGAGGCAAACCTTTCGCCATCGAGGCCGAGAACAGCATC | |
| | CTGGACATCAGCGGCTTCAGCAAGCAGTACAACTGCGCCTTTATTTGGCAGAAGG | |
| | ACGGAGTGAAGAAGCTGAACCTGTACCTGATCATCAACTATTTCAAGGGCGGCAA | |
| | GCTGAGATTCAAGAAGATCAAGCCTGAAGCCTTCGAGGCCAACAGATTCTACACC | |
| | GTGATTAACAAGAAAAGCGGAGAGATCGTGCCAATGGAAGTGAACTTCAACTTCG | |
| | ACGACCCTAACCTGATCATCCTGCCCCTGGCATTTGGCAAGCGGCAGGGCAGAGA | |
| | GTTCATCTGGAACGACCTGCTGTCTCTGGAGACCGGCAGCCTGAAGCTGGCCAAC | |
| | GGCAGAGTGATCGAGAAGACACTGTACAACAGACGAACCAGACAAGACGAGCCCG | |
| | CCCTGTTTGTGGCCCTGACCTTCGAGAGAAGAGAGGTGCTGGACAGCAGCAATAT | |
| | CAAGCCTATGAACCTGATCGGCGTGGACCGGGGCGAGAACATCCCTGCCGTGATC | |
| | GCCCTTACCGACCCCGAGGGATGCCCTCTGAGCCGGTTTAAAGACAGCCTGGGCA | |
| | ACCCTACCCACATCCTGAGAATTGGCGAGTCCTACAAGGAGAAGCAGAGAACCAT | |
| | CCAGGCCAAGAAGGAGGTGGAGCAGCGGCGGGCTGGCGGCTACTCCCGGAAGTAC | |
| | GCCAGCAAGGCCAAGAACCTGGCCGACGACATGGTTAGAAATACCGCCAGAGACC | |
| | TCCTGTACTACGCTGTGACCCAGGACGCCATGCTGATCTTCGAGAACCTGAGCAG | |
| | AGGCTTCGGCAGACAGGGCAAGAGAACCTTCATGGCCGAGAGACAGTACACCCGG | |
| | ATGGAGGACTGGCTGACCGCCAAGCTGGCCTACGAGGGCCTGTCTAAGACCTACC | |
| | TGTCCAAGACCTTGGCACAGTACACCAGCAAGACATGCTCTAACTGCGGCTTCAC | |
| | AATCACGAGCGCCGACTACGACCGGGTGCTGGAGAAACTGAAGAAGACCGCCACA | |
| | GGCTGGATGACCACCATTAACGGCAAGGAGCTGAAGGTGGAGGGCCAGATCACCT | |
| | ACTACAACAGGTACAAACGGCAGAACGTGGTGAAGGACCTGAGCGTGGAACTGGA | |
| | TAGACTGAGCGAGGAAAGCGTAAACAATGACATCAGCAGCTGGACCAAGGGCCGG | |
| | AGCGGCGAGGCCCTGAGCCTGCTGAAGAAGAGATTCTCCCACAGACCAGTGCAGG | |
| | AGAAGTTCGTGTGTCTGAACTGCGGCTTCGAGACCCACGCCGACGAGCAAGCCGC | |
| | CCTGAACATCGCCCGGTCTTGGCTTTTCCTGCGGAGCCAGGAGTACAAGAAGTAC | |
| | CAGACAAACAAGACCACAGGCAACACAGACAAGAGAGCCTTCGTCGAGACCTGGC | |
| | AGAGCTTCTACAGAAAGAAGCTGAAGGAGGTGTGGAAGCCTGCCGTGGGAAGCCC | |
| | CGCTGCCAAGAGAGTGAAGCTGGACTAAGCTGCCTTCTGCGGGGCTTGCCTTCTG | |
| | GCCATGCCCTTCTTCTCTCCCTTGCACCTGTACCTCTTGGTCTTTGAATAAAGCC | |
| | TGAGTAGGAAGTCTAGAAAAAAAAAAAAAAAAAAAAAAAAAAAAAAAAAAAAAAA | |
| | AAAAAAAAAAAAAAAAAAAAAAAAAAAAAAAAAAAAAAAA | |
| 19839 | AGGAAATAAGAGAGAAAAGAAGAGTAAGAAGAAATATAAGAGCCACCATGGCCCC | Full-length |
| | TGCTGCCAAGAGAGTGAAGCTGGATAGCAGACAGGAGATCAAGCGGATTAATAAA | DNA |
| | ATTCGGAGAAGACTGGTGAAGGATTCTAACACAAAGAAGGCTGGCAAGACAGGCC | sequence |
| | CTATGAAGACACTGCTGGTGAGAGTGATGACACCCGACCTGAGAGAAAGACTGGA | encoding |
| | AAACCTGAGAAAGAAGCCTGAGAATATCCCCCAGCCCATCAGCAACACAAGCCGG | CasX 515 |
| | GCCAACCTGAATAAGCTGCTGACCGACTACACCGAAATGAAGAAGGCCATCCTGC | |
| | ACGTGTATTGGGAAGAGTTCCAGAAAGACCCAGTCGGCCTGATGAGCAGAGTGGC | |
| | TCAGCCTGCCAGCAAGAAGATCGATCAGAACAAGCTGAAGCCCGAAATGGACGAG | |
| | AAGGGGAACCTGACAACCGCCGGCTTTGCCTGTAGCCAGTGCGGCCAGCCCCTGT | |
| | TTGTGTACAAACTGGAACAGGTGAGCGAAAAGGGCAAGGCTTACACGAATTACTT | |
| | CGGCAGATGCAACGTGGCCGAGCACGAGAAGCTGATCCTGCTGGCCCAGCTGAAG | |
| | CCTGAGAAGGATAGCGATGAGGCAGTGACATATTCCCTGGGCAAGTTCGGACAGC | |
| | GGGCCCTGGATTTTTATTCCATTCATGTGACCAAGGAATCCACCCACCCCGTCAA | |
| | GCCTCTTGCCCAAATTGCCGGCAACAGATACGCCAGCGGCCCCGTGGGCAAGGCC | |
| | CTGAGCGACGCCTGTATGGGCACCATCGCCAGCTTCCTGTCTAAGTACCAGGACA | |
| | TTATCATCGAGCACCAGAAGGTGGTGAAGGGCAACCAGAAGAGACTGGAGAGCCT | |
| | GCGCGAGCTGGCCGGCAAGGAAAACCTGGAGTATCCTAGCGTGACCCTGCCTCCT | |
| | CAGCCTCATACAAAGGAGGGCGTGGATGCCTACAACGAAGTGATCGCCCGGGTGC | |
| | GGATGTGGGTGAACCTGAATCTGTGGCAGAAGCTGAAGCTGTCTAGAGACGACGC | |
| | CAAGCCCCTGCTGAGACTGAAGGGCTTCCCCAGCTTCCCTCTGGTGGAGAGACAG | |
| | GCAAATGAAGTGGACTGGTGGGACATGGTGTGTAACGTGAAGAAGCTGATCAATG | |
| | AGAAGAAGGAGGACGGCAAAGTGTTCTGGCAGAATCTGGCCGGCTACAAGCGTCA | |
| | GGAGGCCCTGCGGCCCTACCTGAGCAGCGAGGAAGACAGAAAGAAGGGCAAGAAG | |
| | TTCGCCCGGTATCAGCTGGGGGACCTGCTGCTGCACCTCGAGAAGAAGCACGGCG | |
| | AAGACTGGGGGAAGGTGTACGATGAGGCCTGGGAGCGGATCGATAAGAAGGTGGA | |
| | GGGCCTGAGCAAGCACATCAAGCTGGAGGAGGAACGGAGATCTGAGGACGCCCAG | |
| | AGCAAGGCCGCCCTGACCGACTGGCTGAGAGCCAAGGCCAGCTTCGTCATCGAGG | |
| | GGCTGAAGGAGGCCGACAAGGACGAGTTCTGCCGGTGCGAACTGAAGCTGCAGAA | |
| | GTGGTACGGAGATCTGAGAGGCAAACCTTTCGCCATCGAGGCCGAGAACAGCATC | |
| | CTGGACATCAGCGGCTTCAGCAAGCAGTACAACTGCGCCTTTATTTGGCAGAAGG | |
| | ACGGAGTGAAGAAGCTGAACCTGTACCTGATCATCAACTATTTCAAGGGCGGCAA | |
| | GCTGAGATTCAAGAAGATCAAGCCTGAAGCCTTCGAGGCCAACAGATTCTACACC | |
| | GTGATTAACAAGAAAAGCGGAGAGATCGTGCCAATGGAAGTGAACTTCAACTTCG | |
| | ACGACCCTAACCTGATCATCCTGCCCCTGGCATTTGGCAAGCGGCAGGGCAGAGA | |
| | GTTCATCTGGAACGACCTGCTGTCTCTGGAGACCGGCAGCCTGAAGCTGGCCAAC | |
| | GGCAGAGTGATCGAGAAGACACTGTACAACAGACGAACCAGACAAGACGAGCCCG | |
| | CCCTGTTTGTGGCCCTGACCTTCGAGAGAAGAGAGGTGCTGGACAGCAGCAATAT | |
| | CAAGCCTATGAACCTGATCGGCGTGGACCGGGGCGAGAACATCCCTGCCGTGATC | |
| | GCCCTTACCGACCCCGAGGGATGCCCTCTGAGCCGGTTTAAAGACAGCCTGGGCA | |
| | ACCCTACCCACATCCTGAGAATTGGCGAGTCCTACAAGGAGAAGCAGAGAACCAT | |
| | CCAGGCCAAGAAGGAGGTGGAGCAGCGGCGGGCTGGCGGCTACTCCCGGAAGTAC | |
| | GCCAGCAAGGCCAAGAACCTGGCCGACGACATGGTTAGAAATACCGCCAGAGACC | |
| | TCCTGTACTACGCTGTGACCCAGGACGCCATGCTGATCTTCGAGAACCTGAGCAG | |
| | AGGCTTCGGCAGACAGGGCAAGAGAACCTTCATGGCCGAGAGACAGTACACCCGG | |
| | ATGGAGGACTGGCTGACCGCCAAGCTGGCCTACGAGGGCCTGCCCTCTAAGACCT | |
| | ACCTGTCCAAGACCTTGGCACAGTACACCAGCAAGACATGCTCTAACTGCGGCTT | |
| | CACAATCACGAGCGCCGACTACGACCGGGTGCTGGAGAAACTGAAGAAGACCGCC | |
| | ACAGGCTGGATGACCACCATTAACGGCAAGGAGCTGAAGGTGGAGGGCCAGATCA | |
| | CCTACTACAACAGGTACAAACGGCAGAACGTGGTGAAGGACCTGAGCGTGGAACT | |
| | GGATAGACTGAGCGAGGAAAGCGTAAACAATGACATCAGCAGCTGGACCAAGGGC | |
| | CGGAGCGGCGAGGCCCTGAGCCTGCTGAAGAAGAGATTCTCCCACAGACCAGTGC | |
| | AGGAGAAGTTCGTGTGTCTGAACTGCGGCTTCGAGACCCACGCCGACGAGCAAGC | |
| | CGCCCTGAACATCGCCCGGTCTTGGCTTTTCCTGCGGAGCCAGGAGTACAAGAAG | |
| | TACCAGACAAACAAGACCACAGGCAACACAGACAAGAGAGCCTTCGTCGAGACCT | |
| | GGCAGAGCTTCTACAGAAAGAAGCTGAAGGAGGTGTGGAAGCCTGCCGTGGGAAG | |
| | CCCCGCTGCCAAGAGAGTGAAGCTGGACTAAGCTGCCTTCTGCGGGGCTTGCCTT | |
| | CTGGCCATGCCCTTCTTCTCTCCCTTGCACCTGTACCTCTTGGTCTTTGAATAAA | |
| | GCCTGAGTAGGAAGTCTAGAAAAAAAAAAAAAAAAAAAAAAAAAAAAAAAAAAAA | |
| | AAAAAAAAAAAAAAAAAAAAAAAAAAAAAAAAAAAAAAAAAAA | |
| 19840 | AGGAAATAAGAGAGAAAAGAAGAGTAAGAAGAAATATAAGAGCCACCATGGCCCC | Full-length |
| | TGCTGCCAAGAGAGTGAAGCTGGATAGCAGACAGGAGATCAAGCGGATTAATAAA | DNA |
| | ATTCGGAGAAGACTGGTGAAGGATTCTAACACAAAGAAGGCTGGCAAGACACGGG | sequence |
| | GCCCTATGAAGACACTGCTGGTGAGAGTGATGACACCCGACCTGAGAGAAAGACT | encoding |
| | GGAAAACCTGAGAAAGAAGCCTGAGAATATCCCCCAGCCCATCAGCAACACAAGC | CasX 676 |
| | CGGGCCAACCTGAATAAGCTGCTGACCGACTACACCGAAATGAAGAAGGCCATCC | |
| | TGCACGTGTATTGGGAAGAGTTCCAGAAAGACCCAGTCGGCCTGATGAGCAGAGT | |
| | GGCTCAGCCTGCCAGCAAGAAGATCGATCAGAACAAGCTGAAGCCCGAAATGGAC | |
| | GAGAAGGGGAACCTGACAACCGCCGGCTTTGCCTGTAGCCAGTGCGGCCAGCCCC | |
| | TGTTTGTGTACAAACTGGAACAGGTGAGCGAAAAGGGCAAGGCTTACACGAATTA | |
| | CTTCGGCAGATGCAACGTGGCCGAGCACGAGAAGCTGATCAAGCTGGCCCAGCTG | |
| | AAGCCTGAGAAGGATAGCGATGAGGCAGTGACATATTCCCTGGGCAAGTTCGGAC | |
| | AGCGGGCCCTGGATTTTTATTCCATTCATGTGACCAAGGAATCCACCCACCCCGT | |
| | CAAGCCTCTTGCCCAAATTGCCGGCAACAGATACGCCTCCAGCCCCGTGGGCAAG | |
| | GCCCTGAGCGACGCCTGTATGGGCACCATCGCCAGCTTCCTGTCTAAGTACCAGG | |
| | ACATTATCATCGAGCACCAGAAGGTGGTGAAGGGCAACCAGAAGAGACTGGAGAG | |
| | CCTGCGCGAGCTGGCCGGCAAGGAAAACCTGGAGTATCCTAGCGTGACCCTGCCT | |
| | CCTCAGCCTCATACAAAGGAGGGCGTGGATGCCTACAACGAAGTGATCGCCCGGG | |
| | TGCGGATGTGGGTGAACCTGAATCTGTGGCAGAAGCTGAAGCTGTCTAGAGACGA | |
| | CGCCAAGCCCCTGCTGAGACTGAAGGGCTTCCCCAGCTTCCCTCTGGTGGAGAGA | |
| | CAGGCAAATGAAGTGGACTGGTGGGACATGGTGTGTAACGTGAAGAAGCTGATCA | |
| | ATGAGAAGAAGGAGGACGGCAAAGTGTTCTGGCAGAATCTGGCCGGCTACAAGCG | |
| | TCAGGAGGCCCTGCGGCCCTACCTGAGCAGCGAGGAAGACAGAAAGAAGGGCAAG | |
| | AAGTTCGCCCGGTATCAGCTGGGGGACCTGCTGCTGCACCTCGAGAAGAAGCACG | |
| | GCGAAGACTGGGGGAAGGTGTACGATGAGGCCTGGGAGCGGATCGATAAGAAGGT | |
| | GGAGGGCCTGAGCAAGCACATCAAGCTGGAGGAGGAACGGAGATCTGAGGACGCC | |
| | CAGAGCAAGGCCGCCCTGACCGACTGGCTGAGAGCCAAGGCCAGCTTCGTCATCG | |
| | AGGGGCTGAAGGAGGCCGACAAGGACGAGTTCTGCCGGTGCGAACTGAAGCTGCA | |
| | GAAGTGGTACGGAGATCTGAGAGGCAAACCTTTCGCCATCGAGGCCGAGAACAGC | |
| | ATCCTGGACATCAGCGGCTTCAGCAAGCAGTACAACTGCGCCTTTATTTGGCAGA | |
| | AGGACGGAGTGAAGAAGCTGAACCTGTACCTGATCATCAACTATTTCAAGGGCGG | |
| | CAAGCTGAGATTCAAGAAGATCAAGCCTGAAGCCTTCGAGGCCAACAGATTCTAC | |
| | ACCGTGATTAACAAGAAAAGCGGAGAGATCGTGCCAATGGAAGTGAACTTCAACT | |
| | TCGACGACCCTAACCTGATCATCCTGCCCCTGGCATTTGGCAAGCGGCAGGGCAG | |
| | AGAGTTCATCTGGAACGACCTGCTGTCTCTGGAGACCGGCAGCCTGAAGCTGGCC | |
| | AACGGCAGAGTGATCGAGAAGACACTGTACAACAGACGAACCAGACAAGACGAGC | |
| | CCGCCCTGTTTGTGGCCCTGACCTTCGAGAGAAGAGAGGTGCTGGACAGCAGCAA | |
| | TATCAAGCCTATGAACCTGATCGGCGTGGACCGGGGCGAGAACATCCCTGCCGTG | |
| | ATCGCCCTTACCGACCCCGAGGGATGCCCTCTGAGCCGGTTTAAAGACAGCCTGG | |
| | GCAACCCTACCCACATCCTGAGAATTGGCGAGTCCTACAAGGAGAAGCAGAGAAC | |
| | CATCCAGGCCAAGAAGGAGGTGGAGCAGCGGCGGGCTGGCGGCTACTCCCGGAAG | |
| | TACGCCAGCAAGGCCAAGAACCTGGCCGACGACATGGTTAGAAATACCGCCAGAG | |
| | ACCTCCTGTACTACGCTGTGACCCAGGACGCCATGCTGATCTTCGAGAACCTGAG | |
| | CAGAGGCTTCGGCAGACAGGGCAAGAGAACCTTCATGGCCGAGAGACAGTACACC | |
| | CGGATGGAGGACTGGCTGACCGCCAAGCTGGCCTACGAGGGCCTGCCCTCTAAGA | |
| | CCTACCTGTCCAAGACCTTGGCACAGTACACCAGCAAGACATGCTCTAACTGCGG | |
| | CTTCACAATCACGAGCGCCGACTACGACCGGGTGCTGGAGAAACTGAAGAAGACC | |
| | GCCACAGGCTGGATGACCACCATTAACGGCAAGGAGCTGAAGGTGGAGGGCCAGA | |
| | TCACCTACTACAACAGGTACAAACGGCAGAACGTGGTGAAGGACCTGAGCGTGGA | |
| | ACTGGATAGACTGAGCGAGGAAAGCGTAAACAATGACATCAGCAGCTGGACCAAG | |
| | GGCCGGAGCGGCGAGGCCCTGAGCCTGCTGAAGAAGAGATTCTCCCACAGACCAG | |
| | TGCAGGAGAAGTTCGTGTGTCTGAACTGCGGCTTCGAGACCCACGCCGACGAGCA | |
| | AGCCGCCCTGAACATCGCCCGGTCTTGGCTTTTCCTGCGGAGCCAGGAGTACAAG | |
| | AAGTACCAGACAAACAAGACCACAGGCAACACAGACAAGAGAGCCTTCGTCGAGA | |
| | CCTGGCAGAGCTTCTACAGAAAGAAGCTGAAGGAGGTGTGGAAGCCTGCCGTGGG | |
| | AAGCCCCGCTGCCAAGAGAGTGAAGCTGGACTAAGCTGCCTTCTGCGGGGCTTGC | |
| | CTTCTGGCCATGCCCTTCTTCTCTCCCTTGCACCTGTACCTCTTGGTCTTTGAAT | |
| | AAAGCCTGAGTAGGAAGTCTAGAAAAAAAAAAAAAAAAAAAAAAAAAAAAAAAAA | |
| | AAAAAAAAAAAAAAAAAAAAAAAAAAAAAAAAAAAAAAAAAAAAAA | |
| 19841 | AGGAAATAAGAGAGAAAAGAAGAGTAAGAAGAAATATAAGAGCCACCATGGCCCC | Full-length |
| | TGCTGCCAAGAGAGTGAAGCTGGATAGCAGACAGGAGATCAAGCGGATTAATAAA | DNA |
| | ATTCGGAGAAGACTGGTGAAGGATTCTAACACAAAGAAGGCTGGCAAGACAGGCC | sequence |
| | CTATGAAGACACTGCTGGTGAGAGTGATGACACCCGACCTGAGAGAAAGACTGGA | encoding |
| | AAACCTGAGAAAGAAGCCTGAGAATATCCCCCAGCCCATCAGCAACACAAGCCGG | CasX 812 |
| | GCCAACCTGAATAAGCTGCTGACCGACTACACCGAAATGAAGAAGGCCATCCTGC | |
| | ACGTGTATTGGGAAGAGTTCCAGAAAGACCCAGTCGGCCTGATGAGCAGAGTGGC | |
| | TCAGCCTGCCAGCAAGAAGATCGATCAGAACAAGCTGAAGCCCGAAATGGACGAG | |
| | AAGGGGAACCTGACAACCGCCGGCTTTGCCTGTAGCCAGTGCGGCCAGCCCCTGT | |
| | TTGTGTACAAACTGGAACAGGTGAGCGAAAAGGGCAAGGCTTACACGAATTACTT | |
| | CGGCAGATGCAACGTGGCCGAGCACGAGAAGCTGATCCTGCTGGCCCAGCTGAAG | |
| | CCTGAGAAGGATAGCGATGAGGCAGTGACATATTCCCTGGGCAAGTTCGGACAGC | |
| | GGGCCCTGGATTTTTATTCCATTCATGTGACCAAGGAATCCACCCACCCCGTCAA | |
| | GCCTCTTGCCCAAATTGCCGGCAACAGATACGCCAGCGGCCCCGTGGGCAAGGCC | |
| | CTGAGCGACGCCTGTATGGGCACCATCGCCAGCTTCCTGTCTAAGTACCAGGACA | |
| | TTATCATCGAGCACCAGAAGGTGGTGAAGGGCAACCAGAAGAGACTGGAGAGCCT | |
| | GCGCGAGCTGGCCGGCAAGGAAAACCTGGAGTATCCTAGCGTGACCCTGCCTCCT | |
| | CAGCCTCATACAAAGGAGGGCGTGGATGCCTACAACGAAGTGATCGCCCGGGTGC | |
| | GGATGTGGGTGAACCTGAATCTGTGGCAGAAGCTGAAGCTGTCTAGAGACGACGC | |
| | CAAGCCCCTGCTGAGACTGAAGAAGTTCCCCAGCTTCCCTCTGGTGGAGAGACAG | |
| | GCAAATGAAGTGGACTGGTGGGACATGGTGTGTAACGTGAAGAAGCTGATCAATG | |
| | AGAAGAAGGAGGACGGCAAAGTGTTCTGGCAGAATCTGGCCGGCTACAAGCGTCA | |
| | GGAGGCCCTGCGGCCCTACCTGAGCAGCGAGGAAGACAGAAAGAAGGGCAAGAAG | |
| | TTCGCCCGGTATCAGCTGGGGGACCTGCTGCTGCACCTCGAGAAGAAGCACGGCG | |
| | AAGACTGGGGGAAGGTGTACGATGAGGCCTGGGAGCGGATCGATAAGAAGGTGGA | |
| | GGGCCTGAGCAAGCACATCAAGCTGGAGGAGGAACGGAGATCTGAGGACGCCCAG | |
| | AGCAAGGCCGCCCTGACCGACTGGCTGAGAGCCAAGGCCAGCTTCGTCATCGAGG | |
| | GGCTGAAGGAGGCCGACAAGGACGAGTTCTGCCGGTGCGAACTGAAGCTGCAGAA | |
| | GTGGTACGGAGATCTGAGAGGCAAACCTTTCGCCATCGAGGCCGAGAACAGCATC | |
| | CTGGACATCAGCGGCTTCAGCAAGCAGTACAACTGCGCCTTTATTTGGCAGAAGG | |
| | ACGGAGTGAAGAAGCTGAACCTGTACCTGATCATCAACTATTTCAAGGGCGGCAA | |
| | GCTGAGATTCAAGAAGATCAAGCCTGAAGCCTTCGAGGCCAACAGATTCTACACC | |
| | GTGATTAACAAGAAAAGCGGAGAGATCGTGCCAATGGAAGTGAACTTCAACTTCG | |
| | ACGACCCTAACCTGATCATCCTGCCCCTGGCATTTGGCAAGCGGCAGGGCAGAGA | |
| | GTTCATCTGGAACGACCTGCTGTCTCTGGAGACCGGCAGCCTGAAGCTGGCCAAC | |
| | GGCAGAGTGATCGAGAAGACACTGTACAACAGACGAACCAGACAAGACGAGCCCG | |
| | CCCTGTTTGTGGCCCTGACCTTCGAGAGAAGAGAGGTGCTGGACAGCAGCAATAT | |
| | CAAGCCTATGAACCTGATCGGCGTGGACCGGGGCGAGAACATCCCTGCCGTGATC | |
| | GCCCTTACCGACCCCGAGGGATGCCCTCTGAGCCGGTTTAAAGACAGCCTGGGCA | |
| | ACCCTACCCACATCCTGAGAATTGGCGAGTCCTACAAGGAGAAGCAGAGAACCAT | |
| | CCAGGCCAAGAAGGAGGTGGAGCAGCGGCGGGCTGGCGGCTACTCCCGGAAGTAC | |
| | GCCAGCAAGGCCAAGAACCTGGCCGACGACATGGTTAGAAATACCGCCAGAGACC | |
| | TCCTGTACTACGCTGTGACCCAGGACGCCATGCTGATCTTCGAGAACCTGAGCAG | |
| | AGGCTTCGGCAGACAGGGCAAGAGAACCTTCATGGCCGAGAGACAGTACACCCGG | |
| | ATGGAGGACTGGCTGACCGCCAAGCTGGCCTACGAGGGCCTGCCCTCTAAGACCT | |
| | ACCTGTCCAAGACCTTGGCACAGTACACCAGCAAGACATGCTCTAACTGCGGCTT | |
| | CACAATCACGAGCGCCGACTACGACCGGGTGCTGGAGAAACTGAAGAAGACCGCC | |
| | ACAGGCTGGATGACCACCATTAACGGCAAGGAGCTGAAGGTGGAGGGCCAGATCA | |
| | CCTACTACAACAGGTACAAACGGCAGAACGTGGTGAAGGACCTGAGCGTGGAACT | |
| | GGATAGACTGAGCGAGGAAAGCGTAAACAATGACATCAGCAGCTGGACCAAGGGC | |
| | CGGAGCGGCGAGGCCCTGAGCCTGCTGAAGAAGAGATTCTCCCACAGACCAGTGC | |
| | AGGAGAAGTTCGTGTGTCTGAACTGCGGCTTCGAGACCCACGCCGACGAGCAAGC | |
| | CGCCCTGAACATCGCCCGGTCTTGGCTTTTCCTGCGGAGCCAGGAGTACAAGAAG | |
| | TACCAGACAAACAAGACCACAGGCAACACAGACAAGAGAGCCTTCGTCGAGACCT | |
| | GGCAGAGCTTCTACAGAAAGAAGCTGAAGGAGGTGTGGAAGCCTGCCGTGGGAAG | |
| | CCCCGCTGCCAAGAGAGTGAAGCTGGACTAAGCTGCCTTCTGCGGGGCTTGCCTT | |
| | CTGGCCATGCCCTTCTTCTCTCCCTTGCACCTGTACCTCTTGGTCTTTGAATAAA | |
| | GCCTGAGTAGGAAGTCTAGAAAAAAAAAAAAAAAAAAAAAAAAAAAAAAAAAAAA | |
| | AAAAAAAAAAAAAAAAAAAAAAAAAAAAAAAAAAAAAAAAAAA | |

### IV. Guide Nucleic Acids of the Systems for Genetic Editing of Target Nucleic Acid

In another aspect, the present disclosure provides systems comprising a messenger RNA (mRNA) sequence encoding an engineered CasX protein and one or more guide ribonucleic acids (gRNA) for use in modifying an LPA target nucleic acid in a cell. As used herein, the term “gRNA” covers naturally-occurring molecules and gRNA variants, including chimeric gRNA variants comprising domains from different gRNA, gRNAs of the disclosure comprise a scaffold and a targeting sequence complementary to a target nucleic acid of a cell. As used herein, a “system”, used interchangeably with “composition”, can comprise an mRNA encoding an engineered CasX protein and one or more gRNAs of any of the embodiments disclosed herein, which can be utilized as gene editing pairs.

In some embodiments, the disclosure provides systems comprising an mRNA encoding an engineered CasX protein and one or more gRNA as a CasX; gRNA system designed, upon expression of the engineered CasX protein in a transfected cell, to form a ribonucleoprotein (RNP) complex with the gRNA, to target and edit specific locations in the LPA target nucleic acid sequence of the cell. The gRNA provides target specificity to the complex by including a targeting sequence (or “spacer”) having a nucleotide sequence that is complementary to a sequence of the target nucleic acid sequence while the engineered CasX protein of the system provides the site-specific activity such as cleavage or nicking of the target sequence that is guided to a target site (e.g., stabilized at a target site) within a target nucleic acid sequence by virtue of its association with the gRNA. Both single-stranded cleavage and double-stranded cleavage are possible, and double-stranded cleavage can occur as a result of two distinct single-stranded cleavage events by the engineered CasX protein. In some embodiments, indels are introduced in the target nucleic acid by the CasX; gRNA systems of the embodiments described herein and cellular repair systems that can disrupt the protein reading frame of the targeted gene.

Embodiments of gRNAs and formulations of mRNAs and gRNAs for use in the editing of target nucleic acids are described herein, below.

a. Reference gRNA and gRNA Variants

As used herein, a “reference gRNA” refers to a CRISPR guide ribonucleic acid comprising a wild-type sequence of a naturally-occurring gRNA. In some embodiments, a gRNA scaffold of the disclosure may be subjected to one or more mutagenesis methods, such as the mutagenesis methods described in WO2022120095A1 and WO2020247882A1, incorporated by reference herein, which may include Deep Mutational Evolution (DME), deep mutational scanning (DMS), error prone PCR, cassette mutagenesis, random mutagenesis, staggered extension PCR, gene shuffling, domain swapping, or chemical modification to generate one or more gRNA variants with enhanced or varied properties relative to the gRNA scaffold that was modified. The activity of the gRNA scaffold from which a gRNA variant was derived may be used as a benchmark against which the activity of the gRNA variant is compared, thereby measuring improvements in function or other characteristics of the gRNA scaffold.

Table 10 provides the sequences of reference gRNA tracr and scaffold sequences. In some embodiments, the disclosure provides gRNA sequences wherein the gRNA has a scaffold comprising a sequence having one or more nucleotide modifications relative to a reference gRNA sequence of any one of SEQ ID NOS: 451-463 of Table 10.

TABLE 10
Reference gRNA tracr and scaffold sequences

| SEQ ID NO. | Nucleotide Sequence |
|---|---|
| 451 | ACAUCUGGCGCGUUUAUUCCAUUACUUUGGAGCCAGUCCCAGCGACUAUGUCGUAUGGACG |
| | AAGCGCUUAUUUAUCGGAGAGAAACCGAUAAGUAAAACGCAUCAAAG |
| 452 | UACUGGCGCUUUUAUCUCAUUACUUUGAGAGCCAUCACCAGCGACUAUGUCGUAUGGGUAA |
| | AGCGCUUAUUUAUCGGAGAGAAAUCCGAUAAAUAAGAAGCAUCAAAG |
| 453 | ACAUCUGGCGCGUUUAUUCCAUUACUUUGGAGCCAGUCCCAGCGACUAUGUCGUAUGGACG |
| | AAGCGCUUAUUUAUCGGAGA |
| 454 | ACAUCUGGCGCGUUUAUUCCAUUACUUUGGAGCCAGUCCCAGCGACUAUGUCGUAUGGACG |
| | AAGCGCUUAUUUAUCGG |
| 455 | UACUGGCGCUUUUAUCUCAUUACUUUGAGAGCCAUCACCAGCGACUAUGUCGUAUGGGUAA |
| | AGCGCUUAUUUAUCGGAGA |
| 456 | UACUGGCGCUUUUAUCUCAUUACUUUGAGAGCCAUCACCAGCGACUAUGUCGUAUGGGUAA |
| | AGCGCUUAUUUAUCGG |
| 457 | GUUUACACACUCCCUCUCAUAGGGU |
| 458 | GUUUACACACUCCCUCUCAUGAGGU |
| 459 | UUUUACAUACCCCCUCUCAUGGGAU |
| 460 | GUUUACACACUCCCUCUCAUGGGGG |
| 461 | CCAGCGACUAUGUCGUAUGG |
| 462 | GCGCUUAUUUAUCGGAGAGAAAUCCGAUAAAUAAGAAGC |
| 463 | GGCGCUUUUAUCUCAUUACUUUGAGAGCCAUCACCAGCGACUAUGUCGUAUGGGUAAAGCG |
| | CUUAUUUAUCGGA |

b. GRNA Domains and their Function

The gRNAs of the systems of the disclosure comprise two segments: a targeting sequence and a protein-binding segment. The targeting segment of a gRNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a strand of a double stranded target DNA, a target ssRNA, a target ssDNA, etc.), described more fully below. The targeting sequence of a gRNA is capable of binding to a target nucleic acid sequence, including, in the context of the present disclosure, a coding sequence, a complement of a coding sequence, a non-coding sequence, and to accessory elements. The protein-binding segment (or “activator” or “protein-binding sequence”) interacts with (e.g., binds to) a CasX protein as a complex, forming an RNP (described more fully, below). The protein-binding segment is alternatively referred to herein as a “scaffold”, which is comprised of several regions, described more fully, below. The properties and characteristics of CasX gRNA, both wild-type and variants, are described in WO2020247882A1, US20220220508A1, and WO2022120095A1, incorporated by reference herein.

In the case of a reference gRNA, the gRNA occurs naturally as a dual guide RNA (dgRNA), wherein the targeter and the activator portions each have a duplex-forming segment that have complementarity with one another and hybridize to one another to form a double stranded duplex (dsRNA duplex for a gRNA). The term “targeter” or “targeter RNA” is used herein to refer to a crRNA-like molecule (crRNA; “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. In the case of the gRNA for use in the systems of the disclosure, the scaffolds are designed such that the activator and targeter portions are covalently linked to one another (rather than hybridizing to one another) and comprise a single molecule, and can be referred to as a “single-molecule gRNA,” “single guide RNA”, a “single-molecule guide RNA,” a “one-molecule guide RNA”, or a “sgRNA”. The gRNA of the disclosure are all single molecule versions.

Collectively, the assembled gRNAs of the disclosure comprise distinct structured regions, or domains; the RNA triplex, the scaffold stem loop, the extended stem loop, the pseudoknot, and the targeting sequence that, in the embodiments of the disclosure is specific for a target nucleic acid and is located on the 3′ end of the gRNA. The RNA triplex, the scaffold stem loop, the pseudoknot and the extended stem loop, together with the unstructured triplex loop that bridges portions of the triplex, together, are referred to as the “scaffold” of the gRNA. In some cases, the scaffold stem further comprises a bubble. In other cases, the scaffold further comprises a triplex loop region. In still other cases, the scaffold further comprises a 5′ unstructured region. In some embodiments, the gRNA scaffolds of the disclosure for use in the CasX:gRNA systems comprise a scaffold stem loop having the sequence of CCAGCGACUAUGUCGUAGUGG (SEQ ID NO: 542), or a sequence having 1, 2, 3, 4, or 5 mismatches thereto. In some embodiments, the gRNA scaffolds of the disclosure comprise a scaffold stem loop having the sequence of CCAGCGACUAUGUCGUAGUGG (SEQ ID NO: 542) or a sequence having 1, 2, 3, 4, or 5 mismatches thereto.

Each of the structured domains contribute to establish the global RNA fold of the guide and retain functionality of the guide; particularly the ability to properly complex with the CasX protein. For example, the guide scaffold stem interacts with the helical I domain of CasX protein, while residues within the triplex, triplex loop, and pseudoknot stem interact with the OBD of the CasX protein. Together, these interactions confer the ability of the guide to bind and form an RNP with the CasX that retains stability, while the spacer (or targeting sequence) directs and defines the specificity of the RNP for binding a specific sequence of DNA.

Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gRNA and the target nucleic acid sequence. Thus, for example, the gRNA of the disclosure have sequences complementarity to and therefore can hybridize with the target nucleic acid that is adjacent to a sequence complementary to a TC protospacer adjacent motif (PAM) motif or a PAM sequence, such as ATC, CTC, GTC, or TTC. Because the targeting sequence of a guide sequence hybridizes with a sequence of a target nucleic acid sequence, a targeting sequence can be modified by a user to hybridize with a specific target nucleic acid sequence, so long as the location of the PAM sequence is considered. By selection of the targeting sequences of the gRNA, defined regions of the target nucleic acid sequence or sequences bracketing a particular location within the target nucleic acid can be modified or edited using the CasX:gRNA systems described herein. In some embodiments, the targeting sequence of the gRNA has between 15 and 22 consecutive nucleotides. In some embodiments, the targeting sequence has 15, 16, 17, 18, 19, 20, 21, or 22 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides.

By selection of the targeting sequences of the gRNA, defined regions of the target nucleic acid sequence can be modified or edited using the CasX:gRNA systems described herein. In some embodiments, the target nucleic acid comprises a PAM sequence located 5′ of the targeting sequence with at least a single nucleotide separating the PAM from the first nucleotide of the targeting sequence. In some embodiments, the PAM is located on the non-targeted strand of the target region, i.e. the strand that is complementary to the target nucleic acid.

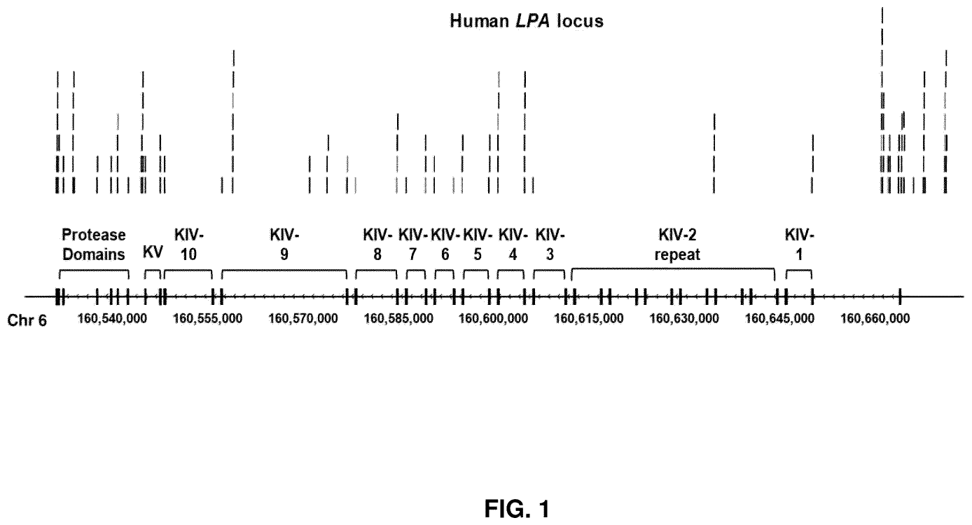

As described herein in Example 1, a computational analysis was performed to identify a set of targeting sequences with desirable characteristics based on their predicted activity and specificity. In some embodiments, the targeting sequence is complementary to a single sequence in the LPA locus; i.e., the sequence is not duplicated in the gene. In some embodiments, the targeting sequence is complementary to a nucleic acid sequence that is unique within the LPA locus. In the foregoing embodiments, the use of a targeting sequence complementary to a single and/or unique sequence in the LPA locus avoids the introduction of multiple breaks in the LPA gene. Sequences within the LPA locus are highly repetitive, with multiple kringle IV (KIV) type 2 domains within the LPA coding sequence (see FIG. 1). Accordingly, without wishing to be bound by theory, it is thought that use of a specific targeting sequence that produces but one break in the LPA gene is important for avoiding chromosomal rearrangements, which may be deleterious. In some embodiments, the targeting sequence for use in the systems of the disclosure is not complementary to a sequence encoding a Kringle domain. The human LPA locus is found on chromosome 6, along with two genes that share a high degree of homologous sequences; the PLG gene, which encoded plasminogen, and the LPA-like pseudogene, LPAL.2. In some embodiments, a targeting sequence is not complementary to the human LPA-like pseudogene, LPAL2. In some embodiments, a targeting sequence is not complementary to human PLG.

In some embodiments, the targeting sequence for use in the systems of the disclosure is complementary to an exon of the LPA gene. In some embodiments, the targeting sequence of the gRNA for use in the systems of the disclosure is complementary to a sequence of an exon selected from the group consisting of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, and exon 39, wherein the identities of the exons correspond to those of human reference genome build hg38. In some embodiments, the targeting sequence of the gRNA for use in the systems of the disclosure is complementary to a sequence of an exon selected from the group consisting of exon 2, exon 17, exon 18, exon 19, exon 20, exon 21, exon 22, exon 23, exon 24, exon 25, exon 26, exon 27, exon 28, exon 29, exon 31, exon 32, exon 33, exon 34, exon 35, exon 36, exon 37, exon 38, and exon 39, wherein the identities of the exons correspond to those of human reference genome build hg38. In some embodiments, the targeting sequence of the gRNA for use in the systems of the disclosure is complementary to a sequence of an exon encoding a Kringle IV (KIV)-1, a KIV-2 I, a KIV-2 II, a KIV-2 III, a KIV-2 IV, a KIV-2 V, a KIV-2 VI, a KIV-3, a KIV-4, a KIV-5, a KIV-6, a KIV-6, a KIV-7, a KIV-8, a KIV-9), a KIV-10, a K V, a Protease 1, a Protease 2, a Protease 3, a Protease 4, a Protease 5, or a Protease 6 domain, wherein the identities of the domains correspond to those of human reference genome build hg38. In some embodiments, the targeting sequence for use in the systems of the disclosure is complementary to an intron of the LPA gene. In some embodiments, the targeting sequence of the gRNA for use in the systems of the disclosure is complementary to a sequence of an intron selected from the group consisting of intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, intron 19, intron 20, intron 21, intron 22, intron 23, intron 24, intron 25, intron 26, intron 27, intron 28, intron 29, intron 30, intron 31, intron 32, intron 33, intron 34, intron 35, intron 36, intron 37, and intron 38, wherein the identities of the introns correspond to those of human reference genome build hg38. In some embodiments, the targeting sequence of the gRNA for use in the systems of the disclosure is complementary to a sequence of an intron selected from the group consisting of intron 1, intron 7, intron 17, intron 18, intron 19, intron 24, intron 26, intron 28, intron 33, intron 37, intron 38, wherein the identities of the introns correspond to those of human reference genome build hg38. In some embodiments, the targeting sequence of the gRNA is complementary to a sequence of an LPA intron-exon junction. In some embodiments, the targeting sequence of the gRNA is complementary to a sequence encoding an untranslated region (UTR) of the LPA gene. In some embodiments, the targeting sequence is complementary to a cis-regulatory element of the LPA gene. In some embodiments, the targeting sequence for use in the systems of the disclosure is complementary to a sequence proximal to the transcription start site of the LPA gene, e.g., within about 300 nucleotides, about 310 nucleotides, about 320 nucleotides, or about 330) nucleotides of the transcription start site. In some embodiments, the targeting sequence for use in the systems of the disclosure is complementary to a sequence that is known to be devoid of a single nucleotide polymorphism (SNP). In some embodiments, the targeting sequence is complementary to a sequence that harbors less than 5% SNP in the LPA gene found in the general human population.

Representative, but non-limiting examples of targeting sequences complementary to LPA target nucleic acids are presented as SEQ ID NOS: 544-19698, and are presented below as Table 11. The targeting sequences for an LPA target nucleic acid presented in Table 11 are suitable for linkage to the gRNA scaffolds of the disclosure; e.g., gRNA 174, 235, or 316, or chemically-modified versions thereof. In some embodiments, the targeting sequence of the gRNA comprises a sequence having at least about 65%, at least about 75%, at least about 85%, or at least about 95% identity to a sequence selected from the group consisting of SEQ ID NOS: 544-19698. In some embodiments, the targeting sequence of the gRNA is selected from the group consisting of SEQ ID NOS: 544-19698, as set forth in Table 11 with 1, 2, 3, 4, or 5 nucleotide(s) removed from the 3′ end of the sequence. In some embodiments, the PAM sequence is ATC. In some embodiments, the targeting sequence for an ATC PAM in the LPA locus comprises SEQ ID NOS: 544-651 and 904-5069, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 544-651 and 904-5069. In some embodiments, the targeting sequence for an ATC PAM in the IPA locus is selected from the group consisting of SEQ ID NOS: 544-651 and 904-5069. In some embodiments, the PAM sequence is CTC. In some embodiments, the targeting sequence for a CTC PAM in the IPA locus comprises SEQ ID NOS: 652-691 and 5070-10230, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 652-691 and 5070-10230. In some embodiments, the targeting sequence for a CTC PAM is selected from the group consisting of SEQ ID NOS: 652-691 and 5070-10230. In some embodiments, the PAM sequence is GTC. In some embodiments, the targeting sequences for a GTC PAM in the LPA locus comprises SEQ ID NOS: 692-748 and 10231-13209 or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 692-748 and 10231-13209. In some embodiments, the targeting sequence for a GTC PAM is selected from the group consisting of SEQ ID NOS: 692-748 and 10231-13209. In some embodiments, the PAM sequence is TTC. In some embodiments, a targeting sequences for a TTC PAM in the LPA locus comprises SEQ ID NOS: 749-903 and 13210-19698, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 749-903 and 13210-19698. In some embodiments, a targeting sequence for a TTC PAM is selected from the group consisting of SEQ ID NOS: 749-903 and 13210-19698.

In some embodiments, the targeting sequence is linked to a gRNA scaffold of the disclosure as described herein. In some embodiments, the gRNA scaffold comprises a sequence of SEQ ID NOS: 464-466, or a chemically modified version thereof.

In some embodiments, the targeting sequence of the gRNA comprises a sequence selected from the group consisting of SEQ ID NOS: 762-903, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical thereto. In some embodiments, a targeting sequence for linkage to a gRNA scaffold is chemically-modified by the addition of a 2′O-methyl group to the last 1, 2, or 3 nucleotides of the 3′ end of the targeting sequence linked to the 3′ end of the scaffold and each of the modified nucleotides is linked to the adjoining nucleotide by a phosphorothioate bond.

In some embodiments, the targeting sequence is complementary to a sequence of an exon, an intron, an exon-intron junction, or a candidate cis-regulatory element (cCRE) of the human LPA gene. In some embodiments, the LPA exon comprises Exon 2, Exon 18, Exon 18, Exon 19, Exon 20, Exon 21, Exon 22, Exon 23, Exon 24, Exon 25, Exon 26, Exon 27, Exon 28, Exon 29, Exon 31, Exon 32, Exon 33, Exon 34, Exon 35, Exon 26, Exon 27, Exon 38, or Exon 39, wherein the identities of the exons correspond to those of human reference genome build hg38. In some embodiments, the targeting sequence complementary to Exon 2 comprises a nucleic acid sequence of SEQ ID NO: 864. In some embodiments, the targeting sequence complementary to Exon 18 comprises a nucleic acid sequence of SEQ ID NOS: 778, 813, 814, 870, or 871. In some embodiments, the targeting sequence complementary to Exon 19 comprises a nucleic acid sequence of SEQ ID NO: 840, 841, 842, 873, or 874. In some embodiments, the targeting sequence complementary to Exon 20 comprises a nucleic acid sequence of SEQ ID NO: 779 or 876. In some embodiments, the targeting sequence complementary to Exon 21 comprises a nucleic acid sequence of SEQ ID NO: 815, 877, or 878. In some embodiments, the targeting sequence complementary to Exon 22 comprises a nucleic acid sequence of SEQ ID NO: 879. In some embodiments, the targeting sequence complementary to Exon 23 comprises a nucleic acid sequence of SEQ ID NO: 780 or 880. In some embodiments, the targeting sequence complementary to Exon 24 comprises a nucleic acid sequence of SEQ ID NO: 881 or 882. In some embodiments, the targeting sequence complementary to Exon 25 comprises a nucleic acid sequence of SEQ ID NO: 816. In some embodiments, the targeting sequence complementary to Exon 26 comprises a nucleic acid sequence of SEQ ID NO: 781, 817, or 884. In some embodiments, the targeting sequence complementary to Exon 27 comprises a nucleic acid sequence of SEQ ID NO: 819. In some embodiments, the targeting sequence complementary to Exon 28 comprises a nucleic acid sequence of SEQ ID NO: 782 or 820. In some embodiments, the targeting sequence complementary to Exon 29 comprises a nucleic acid sequence of SEQ ID NO: 824. In some embodiments, the targeting sequence complementary to Exon 31 comprises a nucleic acid sequence of SEQ ID NO: 787 or 825. In some embodiments, the targeting sequence complementary to Exon 32 comprises a nucleic acid sequence of SEQ ID NO: 788, 789, 826, or 827. In some embodiments, the targeting sequence complementary to Exon 33 comprises a nucleic acid sequence of SEQ ID NO: 890. In some embodiments, the targeting sequence complementary to Exon 35 comprises a nucleic acid sequence of SEQ ID NO: 792, 830, 896, or 897. In some embodiments, the targeting sequence complementary to Exon 36 comprises a nucleic acid sequence of SEQ ID NO: 831 or 898. In some embodiments, the targeting sequence complementary to Exon 37 comprises a nucleic acid sequence of SEQ ID NO: 793 or 832. In some embodiments, the targeting sequence complementary to Exon 38 comprises a nucleic acid sequence of SEQ ID NO: 794 or 901. In some embodiments, the targeting sequence complementary to Exon 38 comprises a nucleic acid sequence of SEQ ID NO: 794 or 901. In some embodiments, the targeting sequence complementary to Exon 39 comprises a nucleic acid of SEQ ID NO: 837.

In some embodiments, the LPA intron comprises Intron 1, Intron 7, Intron 17, intron 18, Intron 19, Intron 24, Intron 26, Intron 28, Intron 33, Intron 37, or Intron 38, wherein the identities of the introns correspond to those of human reference genome build hg38. In some embodiments, the targeting sequence complementary to Intron 1 comprises a nucleic acid sequence of SEQ ID NO: 771, 772, 773, 774, 775, 776, 777, 805, 806, 807, 808, 809, 810, 811, 854, 855, 856, 857, 858, 859, 860, 861, 862, or 863. In some embodiments, the targeting sequence complementary to Intron 7 comprises a nucleic acid sequence oof SEQ ID NOS: 812. 865, 866, or867. In some embodiments, the targeting sequence complementary to Intron 24 comprises a nucleic acid sequence of SEQ ID NO: 883. In some embodiments, the targeting sequence complementary to Intron 28 comprises a nucleic acid sequence of SEQ ID NOS: 783, 784, 785, 786, 821, 822, 823, 885, 886, 887, 888, or 889. In some embodiments, the targeting sequence complementary to intron 33 comprises a nucleic acid sequence of SEQ ID NO: 790, 791 828, 829, 891, 892, 893, 894, or 895. In some embodiments, the targeting sequence complementary to intron 37 comprise a nucleic acid sequence of SEQ ID NO: 833, 834, 835, 843, 844, 889, or 900.

The targeting sequence that may target an exon-intron junction of the human LPA gene and may target one or more of: Exon2-Intron 1, Exon17-Intron 17, Exon 19-Intron 18, Exon20-Intron 19, Exon34-Intron 33, or Exon 39-Intron 38, wherein the identities of the exon-intron junctions correspond to those of human reference genome build hg38. In some embodiments, the targeting sequence complementary to the exon-intron junction of Exon2-Intron 1 comprises a nucleic acid sequence of SEQ ID NO: 862 or 863. In some embodiments, the targeting sequence complementary to the exon-intron junction of Exon17-Intron 17 comprises a nucleic acid sequence of SEQ ID NO: 868. In some embodiments, the targeting sequence complementary to the exon-intron junction of Exon 18-Intron 17 comprises a nucleic acid sequence of SEQ ID NO: 869. In some embodiments, the targeting sequence complementary to the exon-intron junction of Exon 19-Intron 18 comprises a nucleic acid sequence of SEQ ID NO: 872. In some embodiments, the targeting sequence complementary to the exon-intron junction of Exon 20-Intron 19 comprises a nucleic acid sequence of SEQ ID NO: 875. In some embodiments, the targeting sequence complementary to the exon-intron junction of Exon 34-Intron 33 comprises a nucleic acid sequence of SEQ ID NO: 895. In some embodiments, the targeting sequence complementary to the exon-intron junction of Exon 39-Intron 38 comprises a nucleic acid sequence of SEQ ID NO: 836.

In some embodiments, the targeting sequence may target or be complementary to a cCRE of the human LPA gene, wherein the identities of the cCRE correspond to those of human reference genome build hg38 and may comprise a nucleic acid sequence of SEQ ID NOS: 762, 763, 764, 765, 766, 767, 769, 798, 799, 800, 801, 839, 846, 847, 848, 849, 850, 851, 852, or 853. In some embodiments, the targeting sequence may target an untranslated region (UTR) of an exon. In some embodiments the targeting sequence may target or be complementary to the 3′UTR of Exon 39, wherein the identities of exon 39 corresponds to that of human reference genome build hg38 and may comprise a nucleic acid sequence of SEQ ID NO: 795, 796, 797, 838, 845, 902, or 903. In some embodiments, the targeting sequence may comprise a nucleic acid sequence of any nucleic acid sequence of SEQ ID NO: 768, 770, 802, 803, or 804.

In some embodiments, a targeting sequence of the gRNA comprises a sequence selected from the group consisting of SEQ ID NO: 771, SEQ ID NO: 775, SEQ ID NO: 776, SEQ ID NO: 780, SEQ ID NO: 787, SEQ ID NO: 788, SEQ ID NO: 789, SEQ ID NO: 792, SEQ ID NO: 793, SEQ ID NO: 795, SEQ ID NO: 799, SEQ ID NO: 805, SEQ ID NO: 806, SEQ ID NO: 807, SEQ ID NO: 809, SEQ ID NO: 811, SEQ ID NO: 813, SEQ ID NO: 814, SEQ ID NO: 815, SEQ ID NO: 817, SEQ ID NO: 818, SEQ ID NO: 819, SEQ ID NO: 820, SEQ ID NO: 824, SEQ ID NO: 825, SEQ ID NO: 826, SEQ ID NO: 828, SEQ ID NO: 829, SEQ ID NO: 833, SEQ ID NO: 834, SEQ ID NO: 835, SEQ ID NO: 836, SEQ ID NO: 840, SEQ ID NO: 843, SEQ ID NO: 851, SEQ ID NO: 852, SEQ ID NO: 853, SEQ ID NO: 854, SEQ ID NO: 857, SEQ ID NO: 858, SEQ ID NO: 859, SEQ ID NO: 862, SEQ ID NO: 863, SEQ ID NO: 864, SEQ ID NO: 865, SEQ ID NO: 869, SEQ ID NO: 871, SEQ ID NO: 873, SEQ ID NO: 875, SEQ ID NO: 876, SEQ ID NO: 877, SEQ ID NO: 878, SEQ ID NO: 881, SEQ ID NO: 883, SEQ ID NO: 884, SEQ ID NO: 890, SEQ ID NO: 896, SEQ ID NO: 897, SEQ ID NO: 898, and SEQ ID NO: 901, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical thereto. In some embodiments, a targeting sequence for linkage to a gRNA scaffold is chemically-modified by the addition of a 2′O-methyl group to the last 1. 2, or 3 nucleotides of the 3′ end of the targeting sequence linked to the 3′ end of the scaffold and each of the modified nucleotides is linked to the adjoining nucleotide by a phosphorothioate bond.

In some embodiments, a targeting sequence of the gRNA comprises a sequence selected from the group consisting of SEQ ID NO: 776, SEQ ID NO: 792, SEQ ID NO: 817, SEQ ID NO: 826, SEQ ID NO: 878, SEQ ID NO: 896, SEQ ID NO: 898, and SEQ ID NO: 901 or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical thereto.

In some embodiments, the targeting sequence of the gRNA comprises a sequence selected from the group consisting of SEQ ID NO: 817, SEQ ID NO: 826, SEQ ID NO: 898, and SEQ ID NO: 901. In some embodiments, a targeting sequence for linkage to a gRNA scaffold of the disclosure, e.g., gRNA 174, 235, or 316, or chemically-modified version thereof, may comprise the sequence of SEQ ID NO: 901. In some embodiments, a targeting sequence for linkage to a gRNA scaffold of the disclosure, e.g., gRNA 174, 235, or 316, or chemically-modified version thereof, may comprise the sequence of SEQ ID NO: 817. In some embodiments, a targeting sequence for linkage to a gRNA scaffold of the disclosure, e.g., gRNA 174, 235, or 316, or chemically-modified version thereof, may comprise the sequence of SEQ ID NO: 826. In some embodiments, a targeting sequence for linkage to a gRNA scaffold of the disclosure, e.g., gRNA 174, 235, or 316, or chemically-modified version thereof, may comprise the sequence of SEQ ID NO: 898.

In some embodiments, the targeting sequence of the gRNA is chemically-modified by the addition of a 2′O-methyl group to the last 1, 2, or 3 nucleotides of the 3′ end of the targeting sequence linked to the 3′ end of the scaffold and each of the modified nucleotides is linked to the adjoining nucleotide by a phosphorothioate bond. In some embodiments, a chemically modified gRNA with a targeting sequence comprises a sequence selected from the group consisting of SEQ ID NOS: 19855-19862, or a sequence having at least about 70%, at least about 80%, at least about 90%, at least about 95%, at least about 96%, at least about 97%, at least about 98%, at least about 99% sequence identity thereto. In some embodiments, the chemically-modified gRNA targeting an LPA target sequence comprises a sequence selected from the group consisting of SEQ ID NOS: 19855-19862. In some embodiments, the chemically-modified gRNA targeting an LPA target sequence comprises a sequence of SEQ ID NO: 19855. In some embodiments, the chemically-modified gRNA targeting an LPA target sequence comprises a sequence of SEQ ID NO: 19856. In some embodiments, the chemically-modified gRNA targeting an LPA target sequence for use in the LNP comprises a sequence of SEQ ID NO: 19857. In some embodiments, the chemically-modified gRNA targeting an LPA target sequence comprises a sequence of SEQ ID NO: 19858. In some embodiments, the chemically-modified gRNA targeting an LPA target sequence comprises a sequence of SEQ ID NO: 19859. In some embodiments, the chemically-modified gRNA targeting an LPA target sequence comprises a sequence of SEQ ID NO: 19860. In some embodiments, the chemically-modified gRNA targeting an LPA target sequence comprises a sequence of SEQ ID NO: 19861. In some embodiments, the chemically-modified gRNA targeting an LPA target sequence for comprises a sequence of SEQ ID NO: 19862.

In a particular embodiment, the targeting sequence of the gRNA comprises a sequence of SEQ ID NO: 901. In a particular embodiment, the targeting sequence of the gRNA comprises a sequence of SEQ ID NO: 817. In a particular embodiment, the targeting sequence of the gRNA comprises a sequence of SEQ ID NO: 826. In a particular embodiment, the targeting sequence of the gRNA comprises a sequence of SEQ ID NO: 898. In some embodiments, the targeting sequence of SEQ ID NO: 901 is chemically-modified by the addition of a 2′O-methyl group to the last 1, 2, or 3 nucleotides of the 3′ end of the targeting sequence linked to the 3′ end of the scaffold and each of the modified nucleotides is linked to the adjoining nucleotide by a phosphorothioate bond. In some embodiments, a chemically modified gRNA with targeting sequence comprises a sequence of SEQ ID NOS: 19855, or a sequence having at least about 70%, at least about 80%, at least about 90%, at least about 95%, at least about 96%, at least about 97%, at least about 98%, at least about 99% sequence identity thereto.

TABLE 11
RNA Sequences of Targeting Sequences Specific to Human LPA

| SEQ ID NO: | PAM Sequence |
|---|---|
| 544-651, 904-5069 | ATC |
| 749-903, 13210-19698 | TTC |
| 652-691, 5070-10230 | CTC |
| 692-748, 10231-13209 | GTC |

TABLE 12
RNA Sequences of Exemplary Targeting
Sequences Specific to Human LPA

| SEQ ID NO: | PAM Sequence |
|---|---|
| 762-903 | TTC |

In some embodiments, the gRNA and linked targeting sequence used in a system of the disclosure exhibit a low degree of off-target effects to the DNA of a cell. As used herein, “off-target effects” refers to off-target effects of unintended cleavage and mutations at untargeted genomic sites showing a similar but not an identical sequence compared to the target site. In some embodiments, the off-target effects exhibited by a gRNA and linked targeting sequence in a system of the disclosure is less than about 5%, less than about 4%, less than 3%, less than about 2%, less than about 1%, less than about 0.5%, less than 0.1% in cells. In some embodiments the off-target effects are determined in silico. In some embodiments the off-target effects are determined in an in vitro cell-free assay. In some embodiments the off-target effects are determined in a cell-based assay. In some embodiments the off-target effects are determined in a subject administered a therapeutic dose of a CasX:gRNA system of the disclosure.

c, gRNA Modifications

In another aspect, the disclosure relates to gRNA that comprise one or more modifications relative to a gRNA scaffold from which it was derived. In some embodiments, a gRNA variant for use in the systems of the disclosure comprises one or more nucleotide substitutions, insertions, deletions, or swapped or replaced domains relative to a gRNA sequence of the disclosure that improve a characteristic relative to the reference gRNA. Exemplary regions for modifications and swapped regions or domains include the RNA triplex, the pseudoknot, the scaffold stem loop, and the extended stem loop. In some embodiments, the gRNA variant of the disclosure comprises at least a first swapped region from a different gRNA, resulting in a chimeric gRNA. In some embodiments, the gRNA scaffold comprising one or more modifications retains the ability to complex with the engineered CasX described herein as a ribonucleoprotein (RNP). A representative example of such a chimeric gRNA is guide 316 (SEQ ID NO: 466), in which the extended step of gRNA scaffold 235 is replaced with the extended stem of gRNA scaffold 174, wherein the resulting 316 variant retains the ability to form an RNP with a CasX protein and, in some cases, exhibits an improved functional characteristic compared to the parent 235, when assessed in an in vitro or in vivo assay under comparable conditions.

All gRNAs that have one or more improved functions, characteristics, or add one or more new functions when the gRNA scaffold variant is compared to a gRNA scaffold from which it was derived, while retaining the functional properties of being able to complex with the CasX and guide the CasX ribonucleoprotein holo complex to the target nucleic acid are envisaged as within the scope of the disclosure. In some embodiments, the gRNA has an improved characteristic selected from the group consisting of increased editing activity, increased pseudoknot stem stability, increased triplex region stability, increased scaffold stem stability, extended stem stability, reduced off-target folding intermediates, and increased binding affinity to a CasX protein, or any combination thereof. In some cases of the foregoing, the improved characteristic is assessed in an in vitro assay, including the assays of the Examples. In other cases of the foregoing, the improved characteristic is assessed in vivo.

In some embodiments, the gRNA variant scaffold has improved manufacturability compared to the gRNA scaffold from which it was derived. In a particular embodiment, the 316 gRNA scaffold has a shorter sequence compared to the 235 scaffold from which it was derived. In some embodiments, the shorter sequence length of the 316 scaffold confers the improvements of a higher fidelity in the ability to create the guide synthetically with the correct and complete sequence, as well as an enhanced ability to be successfully incorporated into an LNP.

Table 13 provides exemplary gRNA variant scaffold sequences of the disclosure that are utilized as gRNA scaffolds or for the generation of the gRNAs for use in the CasX:gRNA systems of the disclosure. In some embodiments, the gRNA variant scaffold for use in the systems comprises any one of the sequences listed in Table 13 or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto, wherein the gRNA variant retains the ability to form an RNP with a CasX of the disclosure. In other embodiments, the gRNA variant scaffold for use in the CasX:gRNA systems comprises any one of the sequences listed in Table 13 wherein the gRNA variant retains the ability to form an RNP with a CasX of the disclosure. It will be understood that in those embodiments wherein a vector comprises a DNA encoding sequence for a gRNA, that thymine (T) bases can be substituted for the uracil (U) bases of any of the gRNA sequence embodiments described herein.

TABLE 13
Exemplary gRNA Scaffold Sequences

| SEQ ID | Scaffold | |
|---|---|---|
| NO: | variant ID | Nucleotide sequence |
| 464 | 174 | ACUGGCGCUUUUAUCUGAUUACUUUGAGAGCC |
| | | AUCACCAGCGACUAUGUCGUAGUGGGUAAAGCUCCCUCUUCGGAGGGAGCAUCAAAG |
| 465 | 235 | ACUGGCGCUUCUAUCUGAUUACUCUGAGCGCCAUCACCAGCGAC |
| | | UAUGUCGUAGUGGGUAAAGCCGCUUACGGACUUCGGUCCGUAAGAGGCAUCAGAG |
| 466 | 316 | ACUGGCGCUUCUAUCUGAUUACUCUGAGCGCCAUCACCAGCGACUA |
| | | UGUCGUAGUGGGUAAAGCUCCCUCUUCGGAGGGAGCAUCAGAG |

Additional gRNA variants contemplated for use in the systems of the disclosure are selected from the group consisting of SEQ ID NOS: 467-541. Further gRNA variants for use in the systems of the disclosure are described in International Publication Nos. WO2020247882 and WO2022120095, which are hereby incorporated by reference in their entirety.

d, gRNA Scaffold 316

Guide scaffolds can be made by several methods, including recombinantly or by solid-phase RNA synthesis. However, the length of the scaffold can affect the manufacturability when using solid-phase RNA synthesis, with longer lengths resulting in increased manufacturing costs, decreased purity and yield, and higher rates of synthesis failures. For use in lipid nanoparticle (LNP) formulations, solid-phase RNA synthesis of the scaffold is preferred to generate the quantities needed for commercial development. While previous experiments had identified gRNA scaffold 235 as having enhanced properties relative to gRNA scaffold 174, its increased length (in nucleotides) rendered its use for LNP formulations problematic due to synthetic manufacturing constraints. Accordingly, alternative sequences were sought. In some embodiments, the disclosure provides a gRNA wherein the gRNA scaffold and linked targeting sequence has a sequence less than about 115 nucleotides, less than about 110 nucleotides, or less than about 100 nucleotides.

In one embodiment, a gRNA scaffold was designed wherein the scaffold 235 sequence was modified by a domain swap in which the extended stem loop of scaffold 174 replaced the extended stem loop of the 235 scaffold, resulting in the chimeric gRNA scaffold 316, having the sequence ACUGGCGCUUCUAUCUGAUUACUCUGAGCGCCAUCACCAGCGACUAUGUCGUA GUG GGUAAAGCUCCCUCUUCGGAGGGAGCAUCAGAG (SEQ ID NO: 466), having 89 nucleotides, compared with the 99 nucleotides of gRNA scaffold 235. The resulting 316 scaffold had the further advantage in that the extended stem loop does not contain CpG motifs; an enhanced property conferring reduced potential to elicit an immune response. In some embodiments, the disclosure provides gRNA 316 variants that are chemically-modified, described below.

e. Chemically-Modified gRNAs

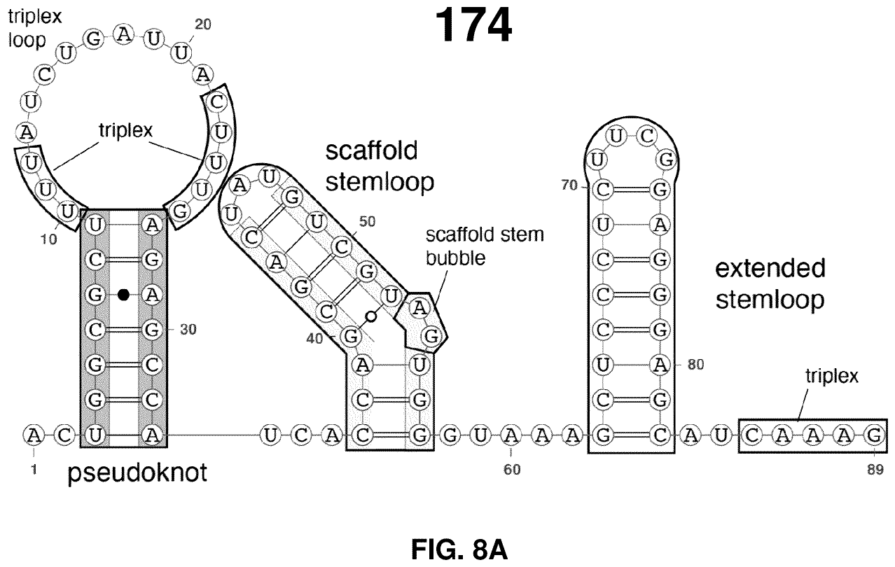

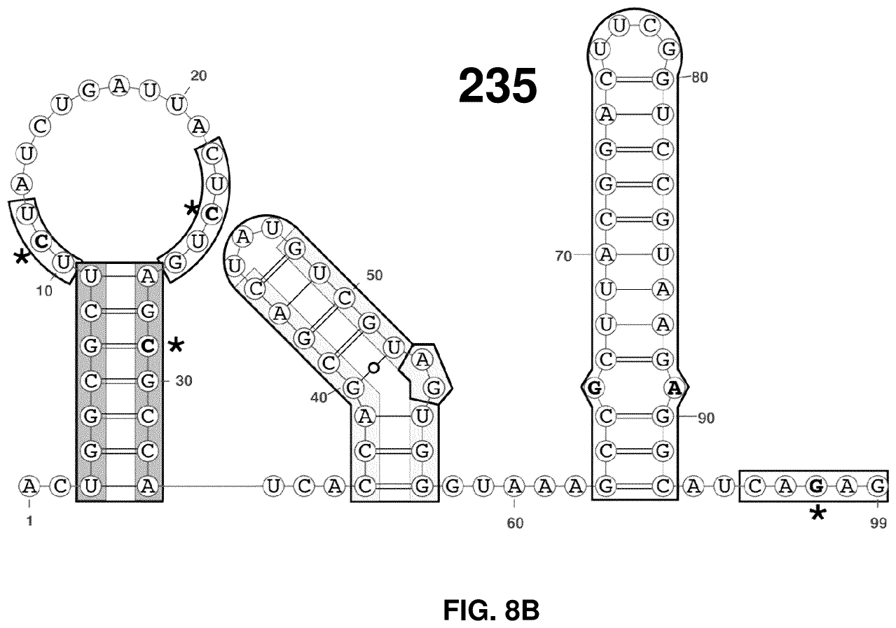

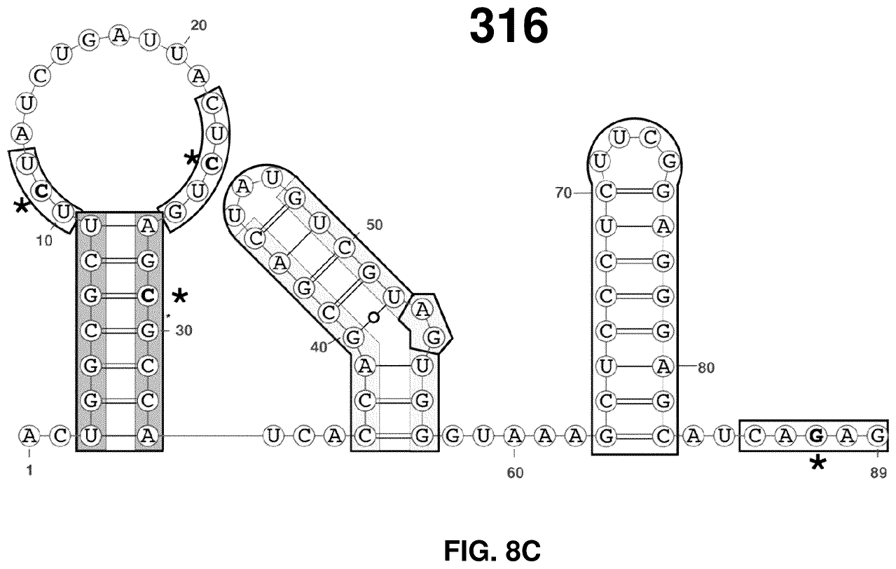

In some embodiments, the gRNAs have one or more chemical modifications. In some embodiments, the chemical modification is the addition of a 2′O-methyl group to one or more nucleotides of the sequence. In some embodiments, the chemical modification is substitution of a phosphorothioate bond between two or more nucleotides of the sequence. In some embodiments, the first 1, 2, or 3 nucleotides of the 5′ end of the scaffold (i.e., A, C, and U in the case of gRNA 174, 235, and 316) are modified by the addition of a 2′O-methyl group and each of the modified nucleotides is linked to the adjoining nucleotide by a phosphorothioate bond. Similarly, the last 1, 2, or 3 nucleotides of the 3′ end of the targeting sequence linked to the 3′ end of the scaffold are similarly modified. In some embodiments, the disclosure provides gRNA with chemical modifications selected from the group consisting of the sequences of SEQ ID NOS: 19704-19712:19714-19722:19724-19732, as set forth in Table 27, or a sequence having at least about 70%, at least about 80%, at least about 90%, at least about 95%, at least about 96%, at least about 97%, at least about 98%, at least about 99% sequence identity thereto. In some embodiments, the gRNA with chemical modifications comprises a scaffold of SEQ ID NOS: 19704-19712:19714-19722:19724-19732, i.e., a sequence of SEQ ID NOS: 19704-19712:19714-19722:19724-19732 without the 20 nucleotides of the 3′ spacer represented in the foregoing sequences as undefined nucleotides. In some embodiments, a gRNA with chemical modifications to the scaffold and a targeting sequence complementary to the LPA gene comprises a sequence selected from the group consisting of SEQ ID NOS: 19855-19862, or a sequence having at least about 70%, at least about 80%, at least about 90%, at least about 95%, at least about 96%, at least about 97%, at least about 98%, at least about 99% sequence identity thereto. In some embodiments, a gRNA with chemical modifications to the scaffold and a targeting sequence complementary to the LPA gene comprises a sequence selected from the group consisting of SEQ ID NOS: 19855-19862. A schematic of the structure of gRNA variants 174, 235, and 316 are shown in FIGS. 8A-8C, respectively. In some embodiments, the gRNA with chemical modifications exhibit improved stability compared to gRNA without chemical modifications.

f. Complex Formation with CasX Protein

Upon delivery or expression of the components of the system in a target cell, the gRNA is capable of complexing as an RNP with a CasX protein and binding to the target nucleic acid of the LPA gene. In some embodiments, a gRNA variant has an improved ability to form an RNP complex with a CasX protein when compared to a reference gRNA or a gRNA variant from which it was derived. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene editing or modification of a target nucleic acid.

### V. Polynucleotides and Vectors

In another aspect, the present disclosure relates to polynucleotides encoding the engineered CasX proteins and gRNAs that have utility in the editing of the LPA gene. Additionally, the disclosure provides vectors comprising polynucleotides encoding the engineered CasX proteins and the gRNAs described herein. In some cases, the vectors are utilized for the expression and recovery of the engineered CasX protein and gRNA components of the gene editing pair. In other cases, the vectors are utilized for the delivery of the encoding polynucleotides to target cells for the editing of the target nucleic acid, as described more fully, below. In some embodiments, sequences encoding the engineered CasX protein and a gRNA are templated on the same vector. In some embodiments, sequences encoding the engineered CasX protein and a gRNA are templated on different vectors. In some embodiments, the gRNA is not encoded by a longer polynucleotide. Suitable vectors are described, for example, in WO2022120095A1 and WO2020247882A1, incorporated by reference herein. As described in WO2022120095A1 and WO2020247882A1, depending on the host/vector system utilized, any of a number of suitable transcription and translation control elements, including constitutive and inducible promoters, transcription enhancer elements, transcription terminators, etc. may be used in the expression vector.

In some embodiments, the disclosure provides polynucleotide sequences encoding the engineered CasX proteins of any of the embodiments described herein, including the engineered CasX proteins of SEQ ID NOS: 4-7 as set forth in Table 1, or sequences having at least about 50%, at least about 60%, at least about 70%, at least about 80%, at least about 90%, at least about 95%, at least about 96%, at least about 97%, at least about 98%, or at least about 99% sequence identity thereto. In some embodiments, the disclosure provides an isolated polynucleotide sequence encoding a gRNA variant of any of the embodiments described herein. In some embodiments, the disclosure provides polynucleotides encoding a gRNA scaffold sequence of SEQ ID NOS: 464-466 as set forth in Table 10, or a sequence having at least about 50%, at least about 60%, at least about 70%, at least about 80%, at least about 90%, at least about 95%, at least about 96%, at least about 97%, at least about 98%, at least about 99% sequence identity thereto, wherein the expressed gRNA variant retains the ability to form an RNP with a CasX. In other embodiments, the disclosure provides polynucleotide sequences encoding gRNA scaffolds of SEQ ID NOS: 464-466 comprising targeting sequences of SEQ ID NOS: 749-903 and 13210-19698, or sequences having at least about 65%, at least about 75%, at least about 85%, or at least about 95% identity thereto.

In some embodiments, the disclosure relates to methods to produce polynucleotide sequences encoding the engineered CasX protein or the gRNA of any of the embodiments described herein, including variants thereof, as well as methods to express the proteins expressed or RNA transcribed by the polynucleotide sequences. In general, the methods include producing a polynucleotide sequence coding for the engineered CasX protein or the gRNA of any of the embodiments described herein and incorporating the encoding gene into an expression vector. In some embodiments, the vector is designed for transduction of cells for modification of the LPA target nucleic acid. Such vectors can include a retroviral vector, a lentiviral vector, an adenoviral vector, an adeno-associated viral (AAV) vector, a herpes simplex virus (HSV) vector, a plasmid, a minicircle, a nanoplasmid, a DNA vector, and an RNA vector. In other embodiments, the expression vector is designed for production of engineered CasX protein, an mRNA encoding CasX, or gRNA in either a cell-free system or in a host cell. For production of the encoded engineered CasX protein or mRNA encoding same, or the gRNA of any of the embodiments described herein in a host cell, the methods include transforming an appropriate host cell with an expression vector comprising the encoding polynucleotide, and culturing the host cell under conditions causing or permitting the resulting engineered CasX protein, mRNA encoding same, or the gRNA of any of the embodiments described herein to be expressed or transcribed in the transformed host cell, thereby producing the engineered CasX protein, mRNA encoding same, or the gRNA, which are recovered by methods described herein (e.g., in the Examples, below) or by standard purification methods known in the art. Standard recombinant techniques in molecular biology are used to make the polynucleotides and expression vectors of the present disclosure.

In accordance with the disclosure, nucleic acid sequences that encode the engineered CasX protein or the gRNA of any of the embodiments described herein are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the engineered CasX protein or the gRNA that is used to transform a host cell for expression of the composition.

In one approach, a construct is first prepared containing the DNA sequence encoding an engineered CasX protein or a gRNA. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the engineered CasX, or the gRNA. Where desired, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NSO cells, SP2/0) cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), yeast cells, or other eukaryotic cells known in the art suitable for the production of recombinant products. Exemplary methods for the creation of expression vectors, the transformation of host cells and the expression and recovery of the engineered CasX protein, mRNA encoding same, or the gRNA are described in the Examples.

The gene encoding the engineered CasX protein or the gRNA construct can be made in one or more steps, either fully synthetically or by synthesis combined with enzymatic processes, such as restriction enzyme-mediated cloning. PCR and overlap extension, including methods more fully described in the Examples. The methods disclosed herein can be used, for example, to ligate sequences of polynucleotides encoding the various components into a gene of a desired sequence. Genes encoding polypeptide compositions are assembled from oligonucleotides using standard techniques of gene synthesis.

In some embodiments, the nucleotide sequence encoding an engineered CasX protein is codon optimized using one or more parameters to enhance expression in the target cell. In some embodiments, the modifications to the mRNA by codon optimization and/or substitution of modified nucleotides in the sequence results in an improved characteristic of at least about 1.1 to about 100,000-fold improved relative to the unmodified mRNA. Non-limiting examples of such parameters include the codon usage in human host cells (e.g., utilizing the codon adaptation index (CAI)), minimizing rare codons, utilizing codon-usage tables derived from biologics intended for use as therapeutics, utilizing an mRNA stability index, or reducing GC content. Methods of codon-optimization, and codon usage in various organisms is known in the art. See, for example, www.genscript.com/tools/codon-frequency-table. This type of optimization can entail a mutation of an encoding nucleotide sequence to mimic the codon preferences of the intended host organism or cell while encoding the same protein. Thus, the codons can be changed, but the encoded protein remains unchanged. For example, if the intended target cell of the engineered CasX protein was a human cell, a human codon-optimized CasX-encoding nucleotide sequence could be used. As another non-limiting example, if the intended host cell were a mouse cell, then a mouse codon-optimized CasX-encoding nucleotide sequence could be generated. The gene design can be performed using algorithms that optimize codon usage and amino acid composition appropriate for the host cell utilized in the production of the engineered CasX protein or the gRNA. In one method of the disclosure, a library of polynucleotides encoding the components of the constructs is created and then assembled, as described above. The resulting genes are then assembled and the resulting genes used to transform a host cell and produce and recover the engineered CasX protein or the gRNA compositions for evaluation of its properties or for use in the modification of the LPA gene, as described herein.

In some embodiments, a nucleotide sequence encoding a gRNA is operably linked to a control element, e.g., a transcriptional control element, such as a promoter. In some embodiments, a nucleotide sequence encoding an engineered CasX protein is operably linked to a control element, e.g., a transcriptional control element, such as a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hepatocytes or a liver sinusoidal endothelial cell.

Non-limiting examples of Pol II promoters operably linked to the polynucleotide encoding the engineered CasX of the disclosure include, but are not limited to EF-1alpha. EF-1 alpha core promoter. Jens Tornoe (JeT), promoters from cytomegalovirus (CMV), CMV immediate early (CMVIE). CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP). CMV promoter full-length promoter, the minimal CMV promoter, the chicken β-actin promoter (CBA). CBA hybrid (CBh), chicken β-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the Ulal small nuclear RNA promoter (226 nt), the Ulal small nuclear RNA promoter (226 nt), the Ulb2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POLI promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In a particular embodiment, the Pol II promoter is EF-1alpha, wherein the promoter enhances transfection efficiency, the transgene transcription or expression of the engineered CasX protein, the proportion of expression-positive clones and the copy number of the episomal vector in long-term culture.

Non-limiting examples of Pol III promoters operably linked to the polynucleotide encoding the gRNA variants of the disclosure include, but are not limited to U6, mini U6. U6 truncated promoters, 7SK, and H1 variants. BiH1 (Bidrectional H1 promoter). BiU6. Bi7SK. BiH1 (Bidirectional U6, 7SK, and H1 promoters), gorilla U6, rhesus U6, human 7SK, human H1 promoters, and truncated versions and sequence variants thereof. In the foregoing embodiment, the pol III promoter enhances the transcription of the gRNA. In a particular embodiment, the Pol III promoter is U6, wherein the promoter enhances expression of the CRISPR gRNA. In another particular embodiment, the promoter linked to the gene encoding the tropism factor is CMV promoter. Experimental details and data for the use of such promoters are provided in the Examples.

Selection of the appropriate vector and promoter is well within the level of ordinary skill in the art, as it related to controlling expression. The expression vector may also contain a ribosome binding site for translation initiation, and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the engineered CasX protein protein that are used for purification or detection.

Recombinant expression vectors of the disclosure can also comprise elements that facilitate robust expression of the proteins and the gRNAs of the disclosure. For example, recombinant expression vectors can include one or more of a polyadenylation signal (poly (A)), an intronic sequence or a post-transcriptional regulatory element such as a woodchuck hepatitis post-transcriptional regulatory element (WPRE). Exemplary poly (A) sequences include hGH poly (A) signal (short), HSV TK poly (A) signal, synthetic polyadenylation signals, SV40 poly (A) signal, β-globin poly (A) signal and the like. A person of ordinary skill in the art will be able to select suitable elements to include in the recombinant expression vectors described herein.

The polynucleotides encoding the engineered CasX proteins or the gRNA sequences can be individually cloned into an expression vector. Selection of the appropriate vector and promoter is well within the level of ordinary skill in the art, as it relates to controlling expression, e.g., for modifying expression of the engineered CasX protein. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, FLAG tag, fluorescent protein, etc.) that can be fused to the CasX protein, thus resulting in a CasX fusion protein that can be used for purification or detection.

The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the engineered CasX protein or an mRNA encoding same can be determined using any nucleic acid or protein assay known in the art. For example, the presence of transcribed mRNA of the engineered CasX protein can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of CasX polynucleotide.

In some embodiments, a vector is created for the transcription of the gene encoding the engineered CasX protein and expression and recovery of the resulting encoding mRNA. In some embodiments, the mRNA is generated by in vitro transcription (IVT) using a PCR product or linearized plasmid DNA template and a T7 RNA polymerase, wherein the plasmid contains a T7 promoter. If using a PCR product, DNA sequences encoding candidate mRNAs will be cloned into a plasmid containing a T7 promoter, wherein the plasmid DNA template will be linearized and then used to perform IVT reactions for expression of the mRNA. Exemplary methods for the generation of such vectors and the production and recovery of the mRNA are provided in the Examples, below.

### VI. Particles for Delivery of CasX:gRNA Systems

In another aspect, the present disclosure provides particle compositions for delivery of the CasX:gRNA systems to cells or to subjects for the modification of the LPA gene. In some embodiments, the disclosure provides synthetic nanoparticles that encapsidate gRNA variants and mRNA encoding engineered CasX proteins of any of the embodiments described herein. In some embodiments, materials for the creation of biodegradable polymeric nanoparticles (PNP) include polylactide, poly (lactic-co-glycolic acid) (PLGA), poly (ethyl cyanoacry late), poly(butyl cyanoacrylate), poly(isobutyl cyanoacrylate), and poly (isohexyl cyanoacry late), polyglutamic acid (PGA), poly (ε-caprolactone) (PCL), cyclodextrin, and natural polymers for instance chitosan, albumin, gelatin, and alginate are the most utilized polymers for the synthesis of PNP (Production and clinical development of nanoparticles for gene delivery. Molecular Therapy-Methods & Clinical Development 3:16023; doi: 10.1038 (2016)). In some embodiments, the disclosure provides virus-like particles for delivery of the engineered CasX protein and gRNA (see, WO2021113772A1, incorporated by reference herein). In other embodiments, the disclosure provides lipid nanoparticles that encapsidate gRNA variants and mRNA encoding engineered CasX proteins of any of the embodiments described herein, described more fully, below. In other embodiments, the particles are lipid nanoparticles that separately encapsulate a gRNA variant and a mRNA encoding engineered CasX proteins of any of the embodiments, and the two types of particles are administered separately.

a. Lipid Nanoparticles (LNP)

In another aspect, the present disclosure provides lipid nanoparticles (LNP) for delivery to cells or to subjects for the modification of the LPA gene. In some embodiments, the LNPs of the disclosure are tissue-specific, have excellent biocompatibility, and can deliver the CasX:gRNA systems with high efficiency, and thus can be used for the modification (e.g. editing) of the target nucleic acid.

In their native forms, nucleic acid polymers are unstable in biological fluids and cannot penetrate into the cytoplasm of target cells, thus requiring delivery systems. Lipid nanoparticles (LNP) have proven useful for both the protection and delivery of nucleic acids to tissues and cells. Furthermore, the use of mRNA in LNPs to encode an engineered CasX protein eliminates the possibility of undesirable genome integration compared to DNA vectors. Moreover. mRNA efficiently transfects both mitotic and non-mitotic cells, as it does not require to enter into the nucleus since it exerts its function in the cytoplasmic compartment. LNPs as a delivery platform offers the additional advantage of being able to co-formulate both the mRNA encoding the nuclease and the gRNA into single LNP particles.

Accordingly, in various embodiments, the disclosure comprises lipid nanoparticles and compositions comprising same that may be used for a variety of purposes, including the delivery of encapsulated or associated (e.g., complexed) therapeutic agents such as nucleic acids to cells, both in vitro and in vivo. In certain embodiments, the disclosure comprises methods of treating or preventing diseases or disorders in a subject in need thereof by contacting the subject with a lipid nanoparticle that encapsulates or is associated with a suitable therapeutic agent, such as the systems, gRNAs and mRNAs described herein.

In certain embodiments, the lipid nanoparticles are useful for the delivery of nucleic acids, including. e.g., the mRNA comprising a sequence encoding the CasX of the disclosure, including the sequences of SEQ ID NOS: 4-7 as set forth in Table 1 and the gRNA embodiments of the disclosure, including the sequences of SEQ ID NOS: 464-466 of Table 13. In some embodiments, the present disclosure provides LNP in which the gRNA and mRNA comprising a sequence encoding the engineered CasX are incorporated into single LNP particles. In other embodiments, the present disclosure provides LNP in which the gRNA and mRNA comprising a sequence encoding the engineered CasX are incorporated into separate LNP particles, which can be formulated together in varying ratios for administration. In some embodiments, the mRNA for incorporation into the LNP of the disclosure comprise a sequence that encodes any of the engineered CasX described herein, including the sequences of SEQ ID NOS: 4-7 as set forth in Table 1. In some embodiments, the mRNA encoding the engineered CasX for incorporation into the LNP of the disclosure comprise the sequences selected from the group consisting of SEQ ID NOS: 19699-19702. 19813-19820, and 19830-19837. In some embodiments, the mRNA encoding the engineered CasX for incorporation into the LNP of the disclosure comprise the sequences selected from the group consisting of SEQ ID NOS: 19700. 19818. 19831 and 19835. In some embodiments, the mRNA further comprises a 5′ cap sequence linked 5′ to the 5′ UTR of the mRNA sequence wherein the 5′ cap has the nucleic acid sequence selected from the group consisting of m7G (5) ppp (5″) mAG, m7G (5′) ppp (5′ (A.G (5′) ppp (5′) A and G (5′) ppp (5′) G. In some embodiments, the gRNA scaffold for use in the LNP comprises the sequence of SEQ ID NO: 466 or a chemically modified version thereof. In some embodiments, the chemically-modified gRNA targeting an LPA target sequence for use in the LNP comprises a sequence selected from the group consisting of SEQ ID NOS: 19855-19862, or a sequence having at least about 70%, at least about 80%, at least about 90%, at least about 95%, at least about 96%, at least about 97%, at least about 98%, at least about 99% sequence identity thereto. In some embodiments, the chemically-modified gRNA targeting an LPA target sequence for use in the LNP comprises a sequence selected from the group consisting of SEQ ID NOS: 19855-19862. In some embodiments, the chemically-modified gRNA targeting an LPA target sequence for use in the LNP comprises a sequence of SEQ ID NO: 19855. In some embodiments, the chemically-modified gRNA targeting an LPA target sequence for use in the LNP comprises a sequence of SEQ ID NO: 19856. In some embodiments, the chemically-modified gRNA targeting an LPA target sequence for use in the LNP comprises a sequence of SEQ ID NO: 19857. In some embodiments, the chemically-modified gRNA targeting an LPA target sequence for use in the LNP comprises a sequence of SEQ ID NO: 19858. In some embodiments, the chemically-modified gRNA targeting an LPA target sequence for use in the LNP comprises a sequence of SEQ ID NO: 19859. In some embodiments, the chemically-modified gRNA targeting an LPA target sequence for use in the LNP comprises a sequence of SEQ ID NO: 19860. In some embodiments, the chemically-modified gRNA targeting an IPA target sequence for use in the LNP comprises a sequence of SEQ ID NO: 19861. In some embodiments, the chemically-modified gRNA targeting an LPA target sequence for use in the LNP comprises a sequence of SEQ ID NO: 19862.

The lipid nanoparticles and compositions of certain embodiments of the disclosure may be used to induce expression of a desired protein both in vitro and in vivo by contacting cells with a lipid nanoparticle comprising one or more novel ionizable lipids described herein, wherein the lipid nanoparticle encapsulates or is associated with a nucleic acid that is expressed to produce the desired protein (e.g., a messenger RNA encoding the engineered CasX protein). In some embodiments, the lipid nanoparticles and compositions may be used to decrease the expression of the LPA target gene both in vitro and in vivo by contacting cells with a lipid nanoparticle comprising one or more novel cationic lipids described herein, wherein the lipid nanoparticle encapsulates or is associated with one or more nucleic acids of the CasX:gRNA systems of the disclosure that modify the target nucleic acid. The lipid nanoparticles and compositions of embodiments of the disclosure may also be used for co-delivery of different nucleic acids (e.g., mRNA and plasmid DNA) separately or in combination, such as may be useful to provide an effect requiring colocalization of different nucleic acids (e.g., mRNA encoding for a suitable gene modifying enzyme and gRNA for targeting of the target nucleic acid).

In some embodiments, the mRNA of the disclosure encoding the engineered CasX protein may be provided in a solution to be mixed with a lipid solution such that the mRNA may be encapsulated in the lipid nanoparticles. A suitable mRNA solution may be any aqueous solution containing mRNA to be encapsulated at various concentrations. For example, a suitable mRNA solution may contain an mRNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.25 mg/ml, 1.5 mg/ml, 1.75 mg/ml, or 2.0 mg/ml. In some embodiments, a suitable mRNA solution may contain an mRNA at a concentration ranging from about 0.01-2.0 mg/ml, 0.01-1.5 mg/ml, 0.01-1.25 mg/ml, 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml.

Early formulations of LNP utilizing permanently cationic lipids resulted in LNPs with positive surface charge that proved toxic in vivo, plus were rapidly cleared by phagocytic cells. By changing to ionizable cationic lipids bearing tertiary or quaternary amines, especially those with pKa <7, resulting LNP achieve efficient encapsulation of nucleic acid polymers at low pH by interacting electrostatically with the negative charges of the phosphate backbone of mRNA, that also result in largely neutral systems at physiological pH values, thus alleviating problems associated with permanently-charged cationic lipids. The lipid composition usually consists of an ionizable amino lipid, a helper lipid (usually a phospholipid), cholesterol, and a polyethylene glycol-lipid conjugate (PEG-lipid) to improve the colloidal stability in biological environments by reducing aspecific absorption of plasma proteins and forming a hydration layer over the nanoparticles, and are formulated at typical mole ratios of 50:10:37-39:1.5-2.5, with variations made to adjust individual properties. Herein, “ionizable lipid” means an amine-containing lipid which can be easily protonated, and for example, it may be a lipid of which charge state changes depending on the surrounding pH. The ionizable lipid may be protonated (positively charged) at a pH below the pKa of a cationic lipid, and it may be substantially neutral at a pH over the pKa. In one example, the LNP may comprise a protonated ionizable lipid and/or an ionizable lipid showing neutrality. In some embodiments, the LNP has a pKa of 5 to 8, 5.5 to 7.5, 6 to 7, or 6.5 to 7. The pKa of the LNP is important for in vivo stability and release of the nucleic acid payload of the LNP. In some embodiments, the LNP having the foregoing pKa ranges may be safely delivered to a target organ (for example, the liver, lung, heart, spleen, as well as to tumors) and/or target cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

The ionizable lipid is an ionizable compound having characteristics similar to lipids generally, and through electrostatic interaction with a nucleic acid (for example, an mRNA of the disclosure), may play a role of encapsulating the nucleic acid within the LNP with high efficiency.

According to the type of the amine comprised in the ionizable lipid, (i) the nucleic acid encapsulation efficiency, (ii) PDI (polydispersity index), and/or (iii) the nucleic acid delivery efficiency to tissue and/or cells constituting an organ (for example, hepatocytes or liver sinusoidal endothelial cells in the liver) of the LNP may be different. In certain embodiments, the ionizable cationic lipid comprises from about 46 mol % to about 66 mol % of the total lipid present in the particle.

The LNP comprising an ionizable lipid comprising an amine may have one or more kinds of the following characteristics: (1) encapsulating a drug with high efficiency; (2) uniform size of prepared particles (or having a low PDI value); and/or (3) excellent nucleic acid delivery efficiency to organs such as liver, lung, heart, spleen, as well as to tumors, and/or cells constituting such organs (for example, hepatocytes, LSEC, cardiac cells, cancer cells, etc.).

The lipid composition usually consists of an ionizable amino lipid, a helper lipid (usually a phospholipid), cholesterol, and a polyethylene glycol-lipid conjugate (PEG-lipid) to improve the colloidal stability in biological environments by reducing aspecific absorption of plasma proteins and forming a hydration layer over the nanoparticles and are formulated at typical mole ratios of 50:10:37-39:1.5-2.5, with variations made to adjust individual properties. As the PEG-lipid forms the surface lipid, the size of the LNP can be readily varied by varying the proportion of surface (PEG) lipid to the core (ionizable cationic) lipids. In some embodiments, the PEG-lipid can be varied from ˜1 to 5 mol % to modify particle properties such as size, stability, and circulation time. In particular, the cationic lipid form plays a crucial role both in nucleic acid encapsulation through electrostatic interactions and intracellular release by disrupting endosomal membranes. The mRNA are encapsulated within the LNP by the ionic interactions they form with the positively charged cationic (or ionizable) lipid. Non-limiting examples of ionizable cationic lipid components utilized in the LNP of the disclosure are selected from DLin-MC3-DMA (heptatriaconta-6,9,28,31-tetraen-19-y14-(dimethylamino) butanoate). DLin-KC2-DMA (2,2-dilinoleyl-4-(2-dimethylaminoethyl)-[1,3]-dioxolane), and TNT (1,3,5-triazinane-2,4,6-trione) and TT (N1,N3,N5-tris(2-aminoethyl)benzene-1,3,5-tricarboxamide). Non-limiting examples of helper lipids utilized in the LNP of the disclosure are selected from DSPC (1,2-distearoyl-sn-glycero-3-phosphocholine). POPC (2-Oleoyl-1-palmitoyl-sn-glycero-3-phosphocholine) and DOPE (1,2-Dioleoyl-sn-glycero-3-phosphoethanolamine). Cholesterol and PEG-DMG ((R)-2,3-bis (octadecyloxy) propyl-1-(methoxy polyethylene glycol 2000) carbamate) or PEG-DSG (1,2-Distearoyl-rac-gly cero-3-methylpolyoxyethylene glycol 2000) are components utilized for the stability, circulation, and size of the LNP.

In other embodiments, the ionizable cationic lipid in the nucleic acid-lipid particles of the disclosure may comprise, for example, one or more ionizable cationic lipids wherein the ionizable cationic lipid is a dialkyl lipid. In another embodiment, the ionizable cationic lipid is a trialkyl lipid. In one particular embodiment, the ionizable cationic lipid is selected from the group consisting of 1,2-dilinoleyloxy-N,N-dimethylaminopropane (DLinDMA), 1,2-dilinolenyloxy-N,N-dimethylaminopropane (DLinDMA), 1,2-di-gamma.-linolenyloxy-N,N-dimethylaminopropane (gamma.-DLinDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), dilinoleylmethyl-3-dimethylaminopropionate (DLin-M-C2-DMA), or salts thereof and mixtures thereof. In a particular embodiment, the ionizable cationic lipid is selected from the group consisting of 1,2-dilinoleyloxy-N,N-dimethylaminopropane (DLinDMA), 1,2-dilinolenyloxy-N,N-dimethylaminopropane (DLenDMA), 1,2-di-, gamma.-linolenyloxy-N, N-dimethylaminopropane (.gamma.-DLenDMA; a salt thereof, or a mixture thereof. In some embodiments, the N/P ratio (nitrogen from the cationic/ionizable lipid and phosphate from the nucleic acid) is in the range of is about 3:1 to 7:1, or about 4:1 to 6:1, or is 3:1, or is 4:1, or is 5:1, or is 6:1, or is 7:1.

The phospholipid of the elements of the LNP according to one example plays a role of covering and protecting a core formed by interaction of the ionizable lipid and nucleic acid in the LNP, and may facilitate cell membrane permeation and endosomal escape during intracellular delivery of the nucleic acid by binding to the phospholipid bilayer of a target cell.

For the phospholipid, a phospholipid which can promote fusion of the LNP according to one example may be used without limitation, and for example, it may be one or more kinds selected from the group consisting of dioleoylphosphatidylethanolamine (DOPE), distearoylphosphatidylcholine (DSPC), palmitoyloleoylphosphatidylcholine (POPC), egg phosphatidylcholine (EPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), phosphatidylethanolamine (PE), dipalmitoylphosphatidylethanolamine, 1,2-dioleoyl-sn-glycero-3-phosphoethanolamine, 1-palmitoyl-2-oleoyl-sn-glycero-3-phosphoethanolamine (POPE), 1-palmitoyl-2-oleoyl-sn-glycero-3-phosphocholine (POPC), 1,2-dioleoyl-sn-glycero-3-[phospho-L-serine] (DOPS), 1,2-dioleoyl-sn-glycero-3-[phospho-L-serine] and the like. In one example, the LNP comprising DOPE may be effective in mRNA delivery (excellent drug delivery efficacy).

The cholesterol of the elements of the LNP according to one example may provide morphological rigidity to lipid filling in the LNP and be dispersed in the core and surface of the nanoparticle to improve the stability of the nanoparticle.

Herein, “lipid-PEG (polyethyleneglycol) conjugate”, “lipid-PEG”, “PEG-lipid”, “PEG-lipid”, or “lipid-PEG” refers to a form in which lipid and PEG are conjugated and means a lipid in which a polyethylene glycol (PEG) polymer which is a hydrophilic polymer is bound to one end. The lipid-PEG conjugate contributes to the particle stability in serum of the nanoparticle within the LNP, and plays a role of preventing aggregation between nanoparticles. In addition, the lipid-PEG conjugate may protect nucleic acids from degrading enzyme during in vivo delivery of the nucleic acids and enhance the stability of nucleic acids in vivo and increase the half-life of the drug encapsulated in the nanoparticle. Examples of PEG-lipid conjugates include, but are not limited to, PEG-DAG conjugates, PEG-DAA conjugates, and mixtures thereof. In certain embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof. In certain embodiments, the PEG-lipid conjugate is a PEG-DAA conjugate. In certain embodiments, the PEG-DAA conjugate in the lipid particle may comprise a PEG-didecyloxypropyl (C10) conjugate, a PEG-dilaury loxypropyl (C12) conjugate, a PEG-dimyristyloxypropyl (C14) conjugate, a PEG-dipalmity loxypropyl (C16) conjugate, a PEG-distearyloxypropyl (C18) conjugate, or mixtures thereof. In certain embodiments, wherein the PEG-DAA conjugate is a PEG-dimyristyloxypropyl (C14) conjugate. In other embodiments, the lipid-PEG conjugate may be PEG bound to phospholipid such as phosphatidylethanolamine (PEG-PE), PEG conjugated to ceramide (PEG-CER, ceramide-PEG conjugate, ceramide-PEG, cholesterol or PEG conjugated to derivative thereof, PEG-c-DOMG, PEG-DMG, PEG-DLPE, PEG-DMPE, PEG-DPPC. PEG-DSPE (DSPE-PEG), and a mixture thereof, and for example, may be C16-PEG2000 ceramide (N-palmitoyl-sphingosine-1-{succinyl[methoxy (polyethylene glycol)2000]}), DMG-PEG 2000, 14:0 PEG2000 PE.

In certain embodiments, the conjugated lipid that inhibits aggregation of particles comprises from about 0).5 mol % to about 3 mol % of the total lipid present in the particle.

In one example, the average molecular weight of the lipid-PEG conjugate may be 100 daltons to 10,000 daltons, 200 daltons to 8,000 daltons, 500 daltons to 5,000 daltons, 1,000 daltons to 3,000 daltons, 1,000 daltons to 2,600 daltons, 1,500 daltons to 2,600 daltons, 1,500 daltons to 2,500 daltons, 2,000 daltons to 2,600 daltons, 2,000 daltons to 2,500 daltons, or 2,000 daltons.

For the lipid in the lipid-PEG conjugate, any lipid capable of binding to polyethyleneglycol may be used without limitation, and the phospholipid and/or cholesterol which are other elements of the LNP may be also used. Specifically, the lipid in the lipid-PEG conjugate may be ceramide, dimyristoylglycerol (DMG), succinoyl-diacylglycerol (s-DAG), distearoylphosphatidylcholine (DSPC), distearoylphosphatidylethanolamine (DSPE), or cholesterol, but not limited thereto.

In the lipid-PEG conjugate, the PEG may be directly conjugated to the lipid or linked to the lipid via a linker moiety. Any linker moiety suitable for binding PEG to the lipid may be used, and for example, includes an ester-free linker moiety and an ester-containing linker moiety. The ester-free linker moiety includes not only amido (—C(O) NH—), amino (—NR—), carbonyl (—C(O)—), carbamate (—NHC (O)O—), urea (—NHC (O) NH—), disulfide (—S—S—), ether (—O—), succinyl (—(O) CCH2CH2C(O)—), succinamidyl (—NHC (O) CH2CH2C (O) NH—), ether, disulfide but also combinations thereof (for example, a linker containing both a carbamate linker moiety and an amido linker moiety), but not limited thereto. The ester-containing linker moiety includes for example, carbonate (—OC (O)O—), succinoyl, phosphate ester (—O—(O) POH—O—), sulfonate ester, and combinations thereof, but not limited thereto.

In certain embodiments, the nucleic acid-lipid particle has a total lipid:mRNA mass ratio of from about 5:1 to about 15:1. In some embodiments, the weight ratio of the ionizable lipid and nucleic acid comprised in the LNP may be 1 to 20:1, 1 to 15:1, 1 to 10:1, 5 to 20:1, 5 to 15:1, 5 to 10:1, 7.5 to 20:1, 7.5 to 15:1, or 7.5 to 10:1.

In one example, the LNP may comprise the ionizable lipid of 20 to 50 parts by weight, phospholipid of 10 to 30 parts by weight, cholesterol of 20 to 60 parts by weight (or 20 to 60 parts by weight), and lipid-PEG conjugate of 0.1 to 10 parts by weight (or 0.25 to 10 parts by weight, 0.5 to 5 parts by weight). The LNP may comprise the ionizable lipid of 20 to 50% by weight, phospholipid of 10 to 30% by weight, cholesterol of 20 to 60% by weight (or 30 to 60% by weight), and lipid-PEG conjugate of 0.1 to 10% by weight (or 0.25 to 10% by weight, 0.5 to 5% by weight) based on the total nanoparticle weight. In other example, the LNP may comprise the ionizable lipid of 25 to 50% by weight, phospholipid of 10 to 20% by weight, cholesterol of 35 to 55% by weight, and lipid-PEG conjugate of 0.1 to 10% by weight (or 0.25 to 10% by weight, 0.5 to 5% by weight), based on the total nanoparticle weight.

In some embodiments, the approach to formulating the LNP of the disclosure (described more fully in the examples) is to dissolve lipids in an organic solvent such as ethanol, which is then mixed through a micromixer with the nucleic acid dissolved in an acidic buffer (usually pH 4). At this pH the ionizable cationic lipid is positively charged and interacts with the negatively-charged nucleic acid polymers. The resulting nanostructures containing the nucleic acids are then converted to neutral LNP when dialyzed against a neutral buffer during the ethanol removal step. The LNP formed by this have a distinct electron-dense nanostructured core where the ionizable cationic lipids are organized into inverted micelles around the encapsulated mRNA molecules, as opposed to the traditional bilayer liposomal structures.

In some embodiments, the LNP may have an average diameter of 20 nm to 200 nm, 20 to 180 nm, 20 nm to 170 nm, 20 nm to 150 nm, 20 nm to 120 nm, 20 nm to 100 nm, 20 nm to 90 nm, 30 nm to 200 nm, 30 to 180 nm, 30 nm to 170 nm, 30 nm to 150 nm, 30 nm to 120 nm, 30 nm to 100 nm, 30 nm to 90 nm, 40 nm to 200 nm, 40 to 180 nm, 40 nm to 170 nm, 40 nm to 150 nm, 40 nm to 120 nm, 40 nm to 100 nm, 40 nm to 90 nm, 40 nm to 80 nm, 40 nm to 70 nm, 50 nm to 200 nm, 50 to 180 nm, 50 nm to 170 nm, 50 nm to 150 nm, 50 nm to 120 nm, 50 nm to 100 nm, 50 nm to 90 nm, 60 nm to 200 nm, 60 to 180 nm, 60 nm to 170 nm, 60 nm to 150 nm, 60 nm to 120 nm, 60 nm to 100 nm, 60 nm to 90 nm, 70 nm to 200 nm, 70 to 180 nm, 70 nm to 170 nm, 70 nm to 150 nm, 70 nm to 120 nm, 70 nm to 100 nm, 70 nm to 90 nm, 80 nm to 200 nm, 80 to 180 nm, 80 nm to 170 nm, 80 nm to 150 nm, 80 nm to 120 nm, 80 nm to 100 nm, 80 nm to 90 nm, 90 nm to 200 nm, 90 to 180 nm, 90 nm to 170 nm, 90 nm to 150 nm, 90 nm to 120 nm, or 90 nm to 100 nm for easy introduction into liver tissue, hepatocytes and/or LSEC (liver sinusoidal endothelial cells). The LNP may be sized for easy introduction into organs or tissues, including but not limited to liver, lung, heart, spleen, as well as to tumors. When the size of the LNP is smaller than the above range, it is difficult to maintain stability as the surface area of the LNP is excessively increased, and thus delivery to the target tissue and/or drug effect may be reduced. The LNP may specifically target liver tissue. The LNP may imitate metabolic behaviors of natural lipoproteins very similarly and may be usefully applied for the lipid metabolism process by the liver and therapeutic mechanism through this. During the drug delivery to hepatocytes or and/or LSEC (liver sinusoidal endothelial cells), the diameter of the fenestrae leading from the sinusoidal lumen to the hepatocytes and LSEC is about 140 nm in mammals and about 100 nm in humans, so the composition for drug delivery having a diameter in the above ranges may have excellent drug delivery efficiency to hepatocytes and LSEC than the LNP having the diameter outside the above range.

According to one example, the LNP comprised in the composition for nucleic acid delivery into target cells may comprise the ionizable lipid: phospholipid: cholesterol: lipid-PEG conjugate in the range described above or at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5. The LNP comprising components at a molar ratio in the above range may have excellent drug delivery efficiency specific to cells of target organs.

The LNP according to one example exhibits a positive charge under the acidic pH condition by showing a pKa of 5 to 8, 5.5 to 7.5, 6 to 7, or 6.5 to 7, and may encapsulate a nucleic acid with high efficiency by easily forming a complex with a nucleic acid through electrostatic interaction with a therapeutic agent such as a nucleic acid showing a negative charge, and it may be usefully used as a composition for intracellular or in vivo drug delivery of a drug (for example, nucleic acid). Herein, “encapsulation” refers to encapsulating a delivery substance for surrounding and embedding it in vivo efficiently, and the drug encapsulation efficiency (encapsulation efficiency) mean the content of the drug encapsulated in the LNP for the total drug content used for preparation.

The encapsulation efficiency of the nucleic acids of the composition in the LNP may be 70% or more, 75% or more, 80% or more, 85% or more, 90% or more, 91% or more, 92% or more, 94% or more, or 95% or more. In other embodiments, the encapsulation efficiency of the nucleic acids of the composition in the LNP is over 80% to 99% or less, over 80% to 97% or less, over 80% to 95% or less, 85% or more to 95% or less, 87% or more to 95% or less, 90% or more to 95% or less, 91% or more to 95% or less, 91% or more to 94% or less, over 91% to 95% or less, 92% or more to 99% or less, 92% or more to 97% or less, or 92% or more to 95% or less. As used herein, “encapsulation efficiency” means the percentage of LNP particles containing the nucleic acids to be incorporated within the LNP. In some embodiments, the mRNA encoding the engineered CasX protein and a gRNA of any of the embodiments of the disclosure are fully encapsulated in the nucleic acid-lipid particle.

The target organs to which a nucleic acid is delivered by the LNP include, but are not limited to the liver, lung, heart, spleen, as well as to tumors. The LNP according to one example is liver tissue-specific and has excellent biocompatibility and can deliver the nucleic acids of a composition with high efficiency, and thus it can be usefully used in related technical fields such as lipid nanoparticle-mediated gene therapy. In a particular embodiment, the target cell to which the nucleic acids are delivered by the LNP according to one example may be a hepatocyte and/or LSEC in vivo. In other embodiments, the disclosure provides LNP formulated for delivery of the nucleic acids of the embodiments to cells ex vivo.

The disclosure also provides a pharmaceutical composition comprising an mRNA encoding an engineered CasX protein and a gRNA variant described herein, and a pharmaceutically acceptable carrier.

In certain embodiments, the nucleic acid-lipid particle has an electron dense core.

In some embodiments, the disclosure provides nucleic acid-lipid particles comprising: (a) an mRNA encoding the engineered CasX and a gRNA variant described herein; (b) one or more ionizable cationic lipids or salts thereof comprising from about 50 mol % to about 85 mol % of the total lipid present in the particle; (c) one or more non-cationic lipids comprising from about 13 mol % to about 49.5 mol % of the total lipid present in the particle; and (d) one or more conjugated lipids that inhibit aggregation of particles comprising from about 0).5 mol % to about 2 mol % of the total lipid present in the particle. In certain embodiments the CasX mRNA and gRNA may be present in the same nucleic acid-lipid particle, or they may be present in different nucleic acid-lipid particles.

In some embodiments, the disclosure provides nucleic acid-lipid particles comprising: (a) an mRNA encoding the CasX described herein; (b) a ionizable cationic lipid or a salt thereof comprising from about 52 mol % to about 62 mol % of the total lipid present in the particle; (c) a mixture of a phospholipid and cholesterol or a derivative thereof comprising from about 36 mol % to about 47 mol % of the total lipid present in the particle; and (d) a PEG-lipid conjugate comprising from about 1 mol % to about 2 mol % of the total lipid present in the particle. In one particular embodiment, the formulation is a four-component system comprising about 1.4 mol % PEG-lipid conjugate (e.g., PEG2000-C-DMA), about 57.1 mol % ionizable cationic lipid (e.g., DLin-K-C2-DMA) or a salt thereof, about 7.1 mol % DPPC (or DSPC), and about 34.3 mol % cholesterol (or derivative thereof).

In another embodiment, the nucleic acid-lipid particle comprises: (a) an mRNA encoding the engineered CasX protein and a gRNA of any of the embodiments described herein; (b) a ionizable cationic lipid or a salt thereof comprising from about 46.5 mol % to about 66.5 mol % of the total lipid present in the particle; (c) cholesterol or a derivative thereof comprising from about 31.5 mol % to about 42.5 mol % of the total lipid present in the particle; and (d) a PEG-lipid conjugate comprising from about 1 mol % to about 2 mol % of the total lipid present in the particle. In one particular embodiment, the formulation is a three-component system which is phospholipid-free and comprises about 1.5 mol % PEG-lipid conjugate (e.g., PEG2000-C-DMA), about 61.5 mol % ionizable cationic lipid (e.g., DLin-K-C2-DMA) or a salt thereof, and about 36.9 mol % cholesterol (or derivative thereof).

Additional formulations are described in PCT Publication No. WO 09/127060 and published U.S. patent application publication numbers US 2011/0071208 A1 and US 2011/0076335 A1, the disclosures of which are herein incorporated by reference in their entirety.

In another embodiment, the nucleic acid-lipid particle comprises: (a) an mRNA encoding the engineered CasX protein and a gRNA of any of the embodiments described herein; (b) one or more ionizable lipids or salts thereof comprising from about 2 mol % to about 50 mol % of the total lipid present in the particle; (c) one or more non-cationic lipids comprising from about 5 mol % to about 90 mol % of the total lipid present in the particle; and (d) one or more conjugated lipids that inhibit aggregation of particles comprising from about 0).5 mol % to about 20 mol % of the total lipid present in the particle.

In another embodiment, the nucleic acid-lipid particle comprises: (a) an mRNA encoding the engineered CasX protein and a gRNA of any of the embodiments described herein; (b) a ionizable cationic lipid or a salt thereof comprising from about 30 mol % to about 50 mol % of the total lipid present in the particle; (c) a mixture of a phospholipid and cholesterol or a derivative thereof comprising from about 47 mol % to about 69 mol % of the total lipid present in the particle; and (d) a PEG-lipid conjugate comprising from about 1 mol % to about 3 mol % of the total lipid present in the particle. In one particular embodiment, the formulation is a four-component system which comprises about 2 mol % PEG-lipid conjugate (e.g., PEG2000-C-DMA), about 40 mol % ionizable cationic lipid (e.g., DLin-K-C2-DMA) or a salt thereof, about 10 mol % DPPC (or DSPC), and about 48 mol % cholesterol (or derivative thereof).

In another embodiment, the nucleic acid-lipid particle comprises: (a) an mRNA encoding the engineered CasX protein and a gRNA of any of the embodiments described herein; (b) one or more ionizable cationic lipids or salts thereof comprising from about 50 mol % to about 65 mol % of the total lipid present in the particle; (c) one or more non-cationic lipids comprising from about 25 mol % to about 45 mol % of the total lipid present in the particle; and (d) one or more conjugated lipids that inhibit aggregation of particles comprising from about 5 mol % to about 10 mol % of the total lipid present in the particle.

In another embodiment, the nucleic acid-lipid particle comprises: (a) an mRNA encoding the engineered CasX protein and a gRNA of any of the embodiments described herein; (b) a ionizable cationic lipid or a salt thereof comprising from about 50 mol % to about 60) mol % of the total lipid present in the particle; (c) a mixture of a phospholipid and cholesterol or a derivative thereof comprising from about 35 mol % to about 45 mol % of the total lipid present in the particle; and (d) a PEG-lipid conjugate comprising from about 5 mol % to about 10 mol % of the total lipid present in the particle.

In certain embodiments, the non-cationic lipid mixture in the formulation comprises: (i) a phospholipid of from about 5 mol % to about 10 mol % of the total lipid present in the particle; and (ii) cholesterol or a derivative thereof of from about 25 mol % to about 35 mol % of the total lipid present in the particle. In one particular embodiment, the formulation is a four-component system which comprises about 7 mol % PEG-lipid conjugate (e.g., PEG750-C-DMA), about 54 mol % ionizable cationic lipid (e.g., DLin-K-C2-DMA) or a salt thereof, about 7 mol % DPPC (or DSPC), and about 32 mol % cholesterol (or derivative thereof).

In another embodiment, the nucleic acid-lipid particle comprises: (a) an mRNA encoding the engineered CasX protein and a gRNA of any of the embodiments described herein; (b) a ionizable cationic lipid or a salt thereof comprising from about 55 mol % to about 65 mol % of the total lipid present in the particle; (c) cholesterol or a derivative thereof comprising from about 30 mol % to about 40 mol % of the total lipid present in the particle; and (d) a PEG-lipid conjugate comprising from about 5 mol % to about 10 mol % of the total lipid present in the particle. In one particular embodiment, the formulation is a three-component system which is phospholipid-free and comprises about 7 mol % PEG-lipid conjugate (e.g., PEG750)—C-DMA), about 58 mol % ionizable cationic lipid (e.g., DLin-K-C2-DMA) or a salt thereof, and about 35 mol % cholesterol (or derivative thereof).

In another embodiment, the nucleic acid-lipid particle comprises: (a) an mRNA encoding the engineered CasX protein and a gRNA of any of the embodiments described herein; (b) a ionizable cationic lipid or a salt thereof comprising from about 48 mol % to about 62 mol % of the total lipid present in the particle; (c) a mixture of a phospholipid and cholesterol or a derivative thereof, wherein the phospholipid comprises about 7 mol % to about 17 mol % of the total lipid present in the particle, and wherein the cholesterol or derivative thereof comprises about 25 mol % to about 40 mol % of the total lipid present in the particle; and (d) a PEG-lipid conjugate comprising from about 0.5 mol % to about 3.0 mol % of the total lipid present in the particle.

### VII. Systems and Methods for Modification of LPA Target Nucleic Acids

The systems encoding or comprising engineered CasX proteins and gRNA of the disclosure have utility in the modification or editing of an LPA target nucleic acid in a population of cells, when used with a guide ribonucleic acid having a targeting sequence complementary to the target nucleic acid to be modified or edited. Such systems are useful for various applications, including as therapeutics, diagnostics, and for research. To effect the methods of the disclosure, resulting in modification of the LPA gene, provided herein are programmable CasX:gRNA systems. The programmable nature of the systems provided herein allows for the precise targeting to achieve the desired effect (nicking, cleaving, modifying, etc.) at one or more regions of predetermined interest in the LPA gene target nucleic acid in a eukaryotic cell. In some embodiments, it may be desirable to knock-down or knock-out expression of the LPA gene in the subject.

A variety of strategies and methods can be employed to modify the target nucleic acid sequence in a cell using the systems provided herein. As used herein “modifying” includes, but is not limited to, cleaving, nicking, editing, deleting, knocking out, knocking down, mutating, exon-skipping and the like. Depending on the system components utilized, the editing event may be a cleavage event followed by introducing random insertions or deletions (indels) or other mutations (e.g., a substitution, duplication, or inversion of one or more nucleotides), for example by utilizing the imprecise non-homologous DNA end joining (NHEJ) repair pathway, which may generate, for example, a frame shift mutation. In some embodiments of the method, the modification comprises introducing an in-frame mutation in the target nucleic acid. In some embodiments of the method, the modification comprises introducing a frame-shifting mutation in the target nucleic acid. In some embodiments of the method, the modification comprises introducing a premature stop codon in the coding sequence in the target nucleic acid. In some embodiments of the method, the modification results in expression of a non-functional protein in the modified cells of the population. As a result of a gene knock-down, the apo (a) protein activity or function may be attenuated or the protein levels may be reduced or eliminated.

In some embodiments, the disclosure provides systems specifically designed for use in the methods to modify the target nucleic acid of an LPA gene in eukaryotic cells; either in vitro, ex vivo, or in vivo in a subject. Generally, any portion of the gene can be targeted using the programmable systems and methods provided herein. In one embodiment, the disclosure provides for a method of modifying a target nucleic acid sequence of a LPA gene in a population of cells, the method comprising introducing into each cell of the population: i) a CasX:gRNA system comprising a CasX and a gRNA of any of the embodiments described herein; ii) a nucleic acid encoding the CasX and gRNA of any of the embodiments described herein; iii) a vector selected from the group consisting of a retroviral vector, a lentiviral vector, an adenoviral vector, an adeno-associated viral (AAV) vector, and a herpes simplex virus (HSV) vector, and comprising the nucleic acid of (iv), above; v) an LNP or a synthetic nanoparticle comprising a gRNA and a mRNA encoding the engineered CasX protein; or vi) combinations of two or more of (i) to (v), wherein the target nucleic acid sequence of the cells targeted by the gRNA is modified by the CasX protein. In some embodiments of the method, the LPA target nucleic acid of at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60% at least about 70%, at least about 80%, or at least about 90% or more of the cells of the population is modified. In some embodiments of the method, the LPA gene in the cells of the population is modified such that expression of the apolipoprotein A is decreased by at least about 10%, at least about 20%, at least about 30%, at least about 40%, at least about 50%, at least about 60%, at least about 70%, at least about 80%, or at least about 90% in comparison to a cell where the LPA gene has not been modified. In some embodiments of the method, the LPA gene of the cells of the population is modified such that at least about 10%, at least about 20%, at least about 30%, at least about 40%, at least about 50%, at least about 60%, at least about 70%, at least about 80%, or at least about 90% of the modified cells do not express a detectable level of apolipoprotein (a). In some embodiments of the method, the LPA gene of the cells of the population is modified such that in the cells of the population such that at least about 10%, at least about 20%, at least about 30%, at least about 40%, at least about 50%, at least about 60%, at least about 70%, at least about 80%, or at least about 90% of the modified cells do not express a detectable level of apolipoprotein (a).

In some embodiments of the method, the modifying of the cell occurs in vitro. In some embodiments of the method, the modifying of the cell occurs ex vivo, wherein the modified cells can be administered to a subject. In some embodiments of the method, the modifying of the cell occurs in vivo. In some embodiments of the method, the cell is a eukaryotic cell. In some embodiments of the method, the eukaryotic cell is selected from the group consisting of a rodent cell, a mouse cell, a rat cell, a primate cell, and a non-human primate cell. In some embodiments of the method, the eukaryotic cell is a human cell. In some embodiments of the method, the cell may be a hepatocyte and/or LSEC (liver sinusoidal endothelial cells).

In some embodiments, the systems provided herein for modification of the target nucleic acid comprise an mRNA encoding an engineered CasX protein comprising a sequence selected from the group consisting of SEQ ID NOS: 19699-19702 and 19830-19837, or a sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto. In some embodiments, the systems provided herein for modification of the target nucleic acid comprise an mRNA encoding an engineered CasX protein comprising a sequence selected from the group consisting of SEQ ID NOS: 19699-19702. In a particular embodiment, the systems are formulated in LNP that encapsulate the mRNA encoding the engineered CasX protein comprising a sequence selected from the group consisting of SEQ ID NOS: 19699-19702 and a gRNA selected from the group consisting of SEQ ID NOS: 19855-19862. In other embodiments, the mRNAs are encoded by DNA that are incorporated into vectors, such as a recombinant Adeno-Associated Viral (AAV) vector, for delivery of the CasX of the disclosure.

In some embodiments of the method, the system is introduced into the cells using LNP comprising mRNA encoding the engineered CasX protein and gRNA of any of the embodiments disclosed herein. LNP components and formulations for use in the system are described herein, below. In some embodiments, the LNP comprises an mRNA encoding the engineered CasX 515 (SEQ ID NO: 5), or a sequence having at least about 70%, at least about 80%, at least about 85%, at least about 90%, at least about 95%, at least about 96%, at least about 97%, at least about 98%, or having at least about 99% sequence identity thereto.

In another embodiment, the LNP comprises an mRNA encoding the engineered CasX 812 (SEQ ID NO: 7), or a sequence having at least about 70%, at least about 80%, at least about 85%, at least about 90%, at least about 95%, at least about 96%, at least about 97%, at least about 98%, or having at least about 99% sequence identity thereto. In another embodiment, the LNP comprises an mRNA encoding the engineered CasX 491 (SEQ ID NO: 4), or a sequence having at least about 70%, at least about 80%, at least about 85%, at least about 90%, at least about 95%, at least about 96%, at least about 97%, at least about 98%, or having at least about 99% sequence identity thereto. In another embodiment, the LNP comprises an mRNA encoding the engineered CasX 676 (SEQ ID NO: 6), or a sequence having at least about 70%, at least about 80%, at least about 85%, at least about 90%, at least about 95%, at least about 96%, at least about 97%, at least about 98%, or having at least about 99% sequence identity thereto. In some embodiments of the foregoing, the LNP further encompass a gRNA variant of the disclosure having a targeting sequence complementary to the target nucleic acid. In some embodiments, the LNP comprises gRNA comprising a sequence of scaffold variant 174 (SEQ ID NO: 464) and a linked targeting sequence complementary to the LPA gene. In some embodiments, the LNP comprises a gRNA comprising a sequence of scaffold variant 235 (SEQ ID NO: 465) and a linked targeting sequence complementary to the LPA gene including a sequence of an exon, an intron, an exon-intron junction, or a cCRE of the human LPA gene, including Exon 2, Exon 18, Exon 18, Exon 19, Exon 20, Exon 21, Exon 22, Exon 23, Exon 24, Exon 25, Exon 26, Exon 27, Exon 28, Exon 29, Exon 31, Exon 32, Exon 33. Exon 34, Exon 35, Exon 26, Exon 27, Exon 38, Exon 39, Intron 1, Intron 7, Intron 17, intron 18, Intron 19, Intron 24, Intron 26, Intron 28, Intron 33, Intron 37, or Intron 38.Exon2-Intron 1, Exon17-Intron 17, Exon 19-Intron 18, Exon20-Intron 19. Exon34-Intron 33, or Exon 39-Intron 38. In some embodiments, the LNP comprises gRNA comprising a sequence of scaffold variant 316 (SEQ ID NO: 466) and a linked targeting sequence complementary to the LPA gene. In some embodiments, the LNP comprises gRNA variant 316 with chemical modifications, including the scaffold portion of the sequences of SEQ ID NOS: 19724-19732 (i.e., the portion without the 20 3′ terminal nucleotides), with a linked targeting sequence complementary to the LPA gene that is also chemically-modified, as described herein. In a particular embodiment, the LNP comprises an mRNA of SEQ ID NO: 19835 encoding the engineered CasX 515 (SEQ ID NO: 5) and gRNA variant 316 with chemical modifications selected from the group consisting of SEQ ID NOS: 19855-19862. In some embodiments of the method, the cells to be modified are selected from the group consisting of rodent cells, mouse cells, rat cells, and non-human primate cells. In other embodiments of the method, the cells to be modified are human cells. In some embodiments of the method, the modification of the population of cells occurs in vivo in a subject, wherein the subject is selected from the group consisting of a rodent, a mouse, a rat, a non-human primate, and a human. In some embodiments of the methods, the modified cell is a hepatocyte, or a cell of the intestine, the kidney, the central nervous system, a smooth muscle cell, a macrophage, a cell of the retina, or a cell of arterial walls such as the endothelium.

The LNP can be administered by a route of administration selected from the group consisting of intravenous, intraarterial, intraportal vein injection, intraperitoneal, intramuscular, intracerebroventricular, intracisternal, intrathecal, intracranial, intralumbar, intraocular, subcutaneous, and oral routes.

The systems and methods described herein can be used to engineer a variety of cells in which expression of the LPA gene is reduced, e.g. knocked-down or knocked-out.

In another aspect, the present disclosure provides particle compositions for delivery of the CasX:gRNA systems described herein, to cells or to subjects for the modification of an LPA gene. Particles envisaged as within the scope of the instant disclosure include, but are not limited to, nanoparticles such as synthetic nanoparticles, polymeric nanoparticles, lipid nanoparticles as described supra, viral particles and virus-like particles. Particles of the disclosure may encapsulate payloads such as gRNA variants, as described herein, optionally in combination with mRNA encoding the CasX proteins of any of the embodiments described herein. Alternatively, or in addition, particles of the disclosure may encapsulate payloads of gRNA and engineered CasX proteins, for example when associated as a ribonucleoprotein (RNP) complex. In some embodiments, the particles are synthetic nanoparticles that encapsulate payloads of gRNA variants and mRNA encoding engineered CasX proteins of any of the embodiments described herein. In some embodiments, the synthetic nanoparticles comprise biodegradable polymeric nanoparticles (PNP). In some embodiments, materials for the creation of biodegradable polymeric nanoparticles (PNP) include polylactide, poly (lactic-co-glycolic acid) (PLGA), poly (ethyl cyanoacrylate), poly(butyl cyanoacrylate), poly (isobutyl cyanoacrylate), and poly (isohexyl cyanoacrylate), polyglutamic acid (PGA), poly (ε-caprolactone) (PCL), cyclodextrin, and natural polymers for instance chitosan, albumin, gelatin, and alginate, which are the most utilized polymers for the synthesis of PNP (Production and clinical development of nanoparticles for gene delivery. Molecular Therapy-Methods & Clinical Development 3:16023; doi: 10.1038 (2016)). In other embodiments, the particles are lipid nanoparticles that encapsulate gRNA variants and mRNA encoding the engineered CasX protein of any of the embodiments described herein, described more fully, below:

a. Lipid Nanoparticle (LNP) Compositions

The present disclosure provides lipid nanoparticles (LNP) for delivery of the CasX:gRNA systems described herein to cells or to subjects for the modification of an LPA gene. In some embodiments, the LNPs of the disclosure are tissue-specific, have excellent biocompatibility, and can deliver the CasX:gRNA systems with high efficiency, and thus can be used for the modification of the targeted gene.

The disclosure further provides LNP compositions and pharmaceutical compositions comprising a plurality of the LNP described herein.

In their native forms, nucleic acid polymers are unstable in biological fluids and cannot penetrate into the cytoplasm of target cells, thus requiring delivery systems. Lipid nanoparticles (LNP) have proven useful for both the protection and delivery of nucleic acids to tissues and cells. Furthermore, the use of mRNA in LNPs to encode the CasX eliminates the possibility of undesirable genome integration, as compared to DNA vectors. Moreover, mRNA efficiently transfects both mitotic and non-mitotic cells, as it does not require entry into the nucleus since it exerts its function in the cytoplasmic compartment. LNPs as a delivery platform thus offer the additional advantage of being able to co-formulate both the mRNA encoding the engineered CasX protein and the gRNA into single LNP particles.

Accordingly, in various embodiments, the disclosure provides lipid nanoparticles and compositions that may be used for a variety of purposes, including the delivery of encapsulated or associated (e.g., complexed) therapeutic agents such as nucleic acids to cells, both in vitro and in vivo. In certain embodiments, the disclosure provides methods of treating or preventing diseases or disorders in a subject in need thereof by contacting the subject with a lipid nanoparticle that encapsulates or is associated with a suitable therapeutic agent complexed through various physical, chemical or electrostatic interactions between one or more of the lipid components used in the compositions to make LNPs. In some embodiments, the suitable therapeutic agent comprises a CasX:gRNA system as described herein.

In certain embodiments, the lipid nanoparticles are useful for the delivery of nucleic acids, including, e.g., the mRNA encoding the engineered CasX proteins of the disclosure, including the sequences of SEQ ID NOS: 19830-19837, and the gRNA variants of the disclosure, including the scaffold sequences of SEQ ID NOS: 464-541, with a linked targeting sequence of the disclosure. In some embodiments, the present disclosure provides LNP in which the gRNA and mRNA encoding the CasX are incorporated into single LNP particles. In other embodiments, the present disclosure provides LNP in which the gRNA and mRNA encoding the engineered CasX protein are incorporated into separate populations of LNPs, which can be formulated together in varying ratios for administration. In some embodiments, the mRNA for incorporation into the LNP comprises a sequence of SEQ ID NOS: 19830-19837. In some embodiments, the gRNA for use in the LNP comprises a scaffold sequence of SEQ ID NOS: 464-541, and the scaffold of SEQ ID NOS: 19703-19732, with a linked targeting sequence of the disclosure. In some embodiments, the gRNA for use in the LNP comprises a sequence of SEQ ID NOS: 19704-19712:19714-19722; or 19724-19732 with a linked targeting sequence of the disclosure. In some embodiments, the gRNA for use in the LNP are chemically-modified. In some embodiments, a chemically-modified gRNA for use in the LNP comprises a sequence selected from the group consisting of SEQ ID NOS: 19855-19862.

The lipid nanoparticles and lipid nanoparticle compositions of certain embodiments of the disclosure may be used to modify expression of a desired protein both in vitro and in vivo by contacting cells with a lipid nanoparticle comprising one or more ionizable lipids described herein, wherein the lipid nanoparticle encapsulates or is associated with a nucleic acid that is expressed to produce the desired protein (e.g., a messenger RNA encoding the CasX protein). In some embodiments, the lipid nanoparticles and compositions may be used to modify the expression of a target gene both in vitro and in vivo by contacting cells with a lipid nanoparticle comprising one or more novel cationic lipids described herein, wherein the lipid nanoparticle encapsulates or is associated with one or more nucleic acids of the CasX:gRNA systems of the disclosure that modify the targeted gene. The lipid nanoparticles and compositions of embodiments of the disclosure may also be used for co-delivery of different nucleic acids (e.g., mRNA and plasmid DNA) separately or in combination, such as may be useful to provide an effect requiring colocalization of different nucleic acids (e.g., mRNA encoding for a suitable gene modifying factor or enzyme and gRNA for targeting of the gene).

In some embodiments, LNPs and LNP compositions described herein include at least one cationic lipid, at least one conjugated lipid, at least one steroid or derivative thereof, at least one additional lipid, or any combination thereof. Alternatively, the lipid compositions of the disclosure can include an ionizable lipid, such as an ionizable cationic lipid, a helper lipid (usually a phospholipid), cholesterol, and a polyethylene glycol-lipid conjugate (PEG-lipid) to improve the colloidal stability in biological environments by, for example, reducing a specific absorption of plasma proteins and forming a hydration layer over the nanoparticles. Such lipid compositions can be formulated at typical mole ratios of 50:10:37-39:1.5-2.5 or 20-50:8-65:25-40:1-2.5, with variations made to adjust individual properties.

The LNPs and LNP compositions of the present disclosure are configured to protect and deliver an encapsulated payload of the systems of the disclosure to tissues and cells, both in vitro and in vivo. Various embodiments of the LNPs and LNP compositions of the present disclosure are described in further detail herein.

### Cationic Lipid

In some aspects, the LNPs and LNP compositions of the present disclosure include at least one cationic lipid. The term “cationic lipid,” refers to a lipid species that has a net positive charge. In some embodiments, the cationic lipid is an ionizable cationic lipid that has a net positive charge at a selected pH, such as physiological pH. In some embodiments, the ionizable cationic lipid has a pKa less than 7 such that the LNPs and LNP compositions achieve efficient encapsulation of the payload at a relatively low pH. In some embodiments, the cationic lipid has a pKa of 5 to 8, 5.5 to 7.5, 6 to 7, or 6.5 to 7. In some embodiments, the cationic lipid may be protonated at a pH below the pKa of the cationic lipid, and it may be substantially neutral at a pH over the pKa. The LNPs and LNP compositions may be safely delivered to a target organ (for example, the liver, lung, heart, spleen, as well as to tumors) and/or cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

Early formulations of LNP utilizing permanently cationic lipids resulted in LNPs with positive surface charge that proved toxic in vivo, plus were rapidly cleared by phagocytic cells. By changing to ionizable cationic lipids bearing tertiary amines, especially those with pKa <7, results in LNP achieving efficient encapsulation of nucleic acid polymers at low pH by interacting electrostatically with the negative charges of the phosphate backbone of mRNA, that also result in largely neutral systems at physiological pH values, thus alleviating problems associated with permanently-charged cationic lipids.

As used herein, “ionizable lipid” means an amine-containing lipid which can be easily protonated, and, for example, it may be a lipid of which charge state changes depending on the surrounding pH. The ionizable lipid may be protonated (positively charged) at a pH below the pKa of a cationic lipid, and it may be substantially neutral at a pH over the pKa. In one example, the LNP may comprise a protonated ionizable lipid and/or an ionizable lipid showing neutrality. In some embodiments, the LNP has a pKa of 5 to 8, 5.5 to 7.5, 6 to 7, or 6.5 to 7. The pKa of the LNP is important for in vivo stability and release of the nucleic acid payload of the LNP in the target cell or organ. In some embodiments, the LNP having the foregoing pKa ranges may be safely delivered to a target organ (for example, the liver, lung, heart, spleen, as well as to tumors) and/or target cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

The ionizable lipid is an ionizable compound having characteristics similar to lipids generally, and when the pH of the formulation buffer is kept below its pKa, the ionizable lipid through electrostatic interaction with a nucleic acid (for example, an mRNA of the disclosure), may play a role of encapsulating the nucleic acid payloads within the LNP with high efficiency.

According to the type of the amine and the tail group comprised in the ionizable lipid, (i) the nucleic acid encapsulation efficiency, (ii) PDI (polydispersity index), and/or (iii) the nucleic acid delivery efficiency to tissue and/or cells constituting an organ (for example, hepatocytes or liver sinusoidal endothelial cells in the liver) of the LNP may be different. In certain embodiments, the ionizable lipid is an ionizable cationic lipid, and comprises from about 46 mol % to about 66 mol % of the total lipid present in the particle.

The LNP comprising an ionizable lipid comprising an amine may have one or more kinds of the following characteristics: (1) the ability to encapsulate a nucleic acid with high efficiency; (2) uniform size of prepared particles (or having a low PDI value); and/or (3) excellent nucleic acid delivery efficiency to organs such as liver, lung, heart, spleen, bone marrow, as well as to tumors, and/or cells constituting such organs (for example, hepatocytes, LSEC, cardiac cells, cancer cells, etc.).

In particular embodiments, the cationic lipid form plays a crucial role both in nucleic acid encapsulation through electrostatic interactions and intracellular release by disrupting endosomal membranes. The nucleic acid payloads are encapsulated within the LNP by the ionic interactions they form with the positively charged cationic lipid. Non-limiting examples of cationic lipid components utilized in the LNP of the disclosure are selected from DLin-MC3-DMA (heptatriaconta-6,9,28,31-tetraen-19-v14-(dimethylamino) butanoate), DLin-KC2-DMA (2,2-dilinoleyl-4-(2-dimethylaminoethyl)-[1,3]-dioxolane), and TNT (1,3,5-triazinane-2,4,6-trione) and TT (N1,N3,N5-tris(2-aminoethyl)benzene-1,3,5-tricarboxamide). Non-limiting examples of helper lipids utilized in the LNP of the disclosure are selected from DSPC (1,2-distearoyl-sn-glycero-3-phosphocholine), POPC (2-Oleoyl-1-palmitoyl-sn-glycero-3-phosphocholine) and DOPE (1,2-Dioleoyl-sn-glycero-3-phosphoethanolamine), 1,2-dioleoyl-sn-glycero-3-phospho-(1′-rac-glycerol) DOPG, 1,2-Dimyristoyl-sn-glycero-3-phosphoethanolamine (DMPE), 1,2-dilauroyl-sn-glycero-3-phosphocholine (DLPC), sphingolipid, and ceramide. Cholesterol and PEG-DMG ((R)-2,3-bis (octadecy loxy) propyl-1-(methoxy polyethylene glycol 2000) carbamate), PEG-DSG (1,2-Distearoyl-rac-glycero-3-methylpolyoxyethylene glycol 2000), or DSPE-PEG2k (1,2-distearoyl-sn-glycero-3-phosphoethanolamine-N-[amino (polyethylene glycol)-2000]), are components utilized in the LNP of the disclosure for the stability, circulation, and size of the LNP.

In some embodiments, the cationic lipid in the LNP of the disclosure comprises a tertiary amine. In some embodiments, the tertiary amine includes alkyl chains connected to N of the tertiary amine with ether linkages. In some embodiments, the alkyl chains comprise C12-C30 alkyl chains having 0 to 3 double bonds. In some embodiments, the alkyl chains comprise C16-C22 alkyl chains. In some embodiments, the alkyl chains comprise C18 alkyl chains. A number of cationic lipids and related analogs have been described in U.S. Patent Publication Nos. 20060083780, 20060240554, 20110117125, 20190336608, 20190381180 and 20200121809; U.S. Pat. Nos. 5,208,036; 5,264,618:5,279,833:5,283,185:5,753,613:5, 785,992:9,738,593:10,106,490; 10,166,298:10,221,127; and 11,219,634; and PCT Publication No. WO 96/10390, the disclosures of which are herein incorporated by reference in their entirety.

In some embodiments, the cationic lipid in the LNP of the disclosure may comprise, for example, one or more ionizable cationic lipids wherein the ionizable cationic lipid is a dialkyl lipid. In other embodiments, the ionizable cationic lipid is a trialkyl lipid.

In some embodiments, the cationic lipid in the LNP of the disclosure is selected from 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N—(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxy butan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzyllamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), and any combination of the forgoing.

In some embodiments, the cationic lipid in the LNP of the disclosure is selected from heptatriaconta-6,9,28,31-tetraen-19-yl4-(dimethylamino) butanoate (DLin-MC3-DMA), 2,2-dilinoleyl-4-(2-dimethylaminoethyl)-[1,3]-dioxolane (DLin-KC2-DMA), (1,3,5-triazinane-2,4,6-trione) (TNT), N1,N3,N5-tris(2-aminoethyl)benzene-1,3,5-tricarboxamide (TT), and any combination of the forgoing.

In some embodiments, the N/P ratio (nitrogen from the cationic/ionizable lipid and phosphate from the nucleic acid) in the LNP of the disclosure is in the range of is about 3:1 to 7:1, or about 4:1 to 6:1, or is 3:1, or is 4:1, or is 5:1, or is 6:1, or is 7:1.

### Conjugated Lipid

In some embodiments, the LNPs and LNP compositions of the present disclosure include at least one conjugated lipid. In some embodiments, the conjugated lipid may be selected from a polyethyleneglycol (PEG)-lipid conjugate, a polyamide (ATTA)-lipid conjugate, a cationic-polymer-lipid conjugate (CPL), and any combination of the foregoing. In some cases, conjugated lipids can inhibit aggregation of the LNPs of the disclosure.

In some embodiments, the conjugated lipid of the LNP of the disclosure comprises a pegylated lipid. The terms “polyethyleneglycol (PEG)-lipid conjugate,” “pegylated lipid” “lipid-PEG conjugate”, “lipid-PEG”, “PEG-lipid”, “PEG-lipid”, or “lipid-PEG” are used interchangeably herein and refer to a lipid attached to a polyethylene glycol (PEG) polymer which is a hydrophilic polymer. The pegylated lipid contributes to the stability of the LNPs and LNP compositions and reduces aggregation of the LNPs.

As the PEG-lipid can form the surface lipid, the size of the LNP can be readily varied by varying the proportion of surface (PEG) lipid to the core (ionizable cationic) lipids. In some embodiments, the PEG-lipid of the LNP of the disclosure can be varied from ˜1 to 5 mol % to modify particle properties such as size, stability, and circulation time.

The lipid-PEG conjugate contributes to the particle stability in serum of the nanoparticle within the LNP and plays a role of preventing aggregation between nanoparticles. In addition, the lipid-PEG conjugate may protect nucleic acids, such as mRNAs encoding the CasX proteins of the disclosure, or gRNAs of the disclosure, from degrading enzymes during in vivo delivery of the nucleic acids and enhance the stability of the nucleic acids in vivo and increase the half-life of the delivered nucleic acids encapsulated in the nanoparticle. Examples of PEG-lipid conjugates include, but are not limited to, PEG-DAG conjugates, PEG-DAA conjugates, and mixtures thereof. In certain embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof.

In some embodiments, the pegylated lipid of the LNP of the disclosure is selected from a PEG-ceramide, a PEG-diacylglycerol, a PEG-dialkyloxypropyl, a PEG-dialkoxypropylcarbamate, a PEG-phosphatidylethanoloamine, a PEG-phospholipid, a PEG-succinate diacylglycerol, and any combination of the foregoing.

In some embodiments, the pegylated lipid of the LNP of the disclosure is a PEG-dialkyloxypropyl. In some embodiments, the pegylated lipid is selected from PEG-didecy loxypropyl (C10), PEG-dilauryloxypropyl (C12), PEG-dimyristyloxypropyl (C14), PEG-dipalmityloxypropyl (C16), PEG-distearyloxypropyl (C18), and any combination of the foregoing.

In other embodiments, the lipid-PEG conjugate of the LNP of the disclosure may be PEG bound to phospholipid such as phosphatidylethanolamine (PEG-PE), PEG conjugated to ceramide (PEG-CER, ceramide-PEG conjugate, ceramide-PEG, cholesterol or PEG conjugated to derivative thereof, PEG-c-DOMG, PEG-DMG, PEG-DLPE, PEG-DMPE, PEG-DPPC, PEG-DSPE (DSPE-PEG), and a mixture thereof, and for example, may be C16-PEG2000 ceramide (N-palmitoyl-sphingosine-1-{succinyl [methoxy (polyethylene glycol) 2000]}), DMG-PEG 2000, 14:0 PEG2000 PE.

In some embodiments, the pegylated lipid of the LNP of the disclosure is selected from 1-(monomethoxy-polyethyleneglycol)-2,3-dimyristoylglycerol, 4-O-(2′,3′-di(tetradecanoyloxy) propyl-1-O-(ω-methoxy (polyethoxy)ethyl) butanedioate (PEG-S-DMG), ω-methoxy (polyethoxy)ethyl-N-(2,3-di(tetradecanoxy) propyl) carbamate, 2,3-di (tetradecanoxy) propyl-N-(ω-methoxy (polyethoxy)ethyl) carbamate, and any combination of the foregoing.

In some embodiments, the pegylated lipid of the LNP of the disclosure is selected from mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-w-methyl-poly (ethylene glycol) (2 KPEG-DMG), and any combination of the foregoing.

In some embodiments, the PEG is directly attached to the lipid of the pegylated lipid. In other embodiments, the PEG is attached to the lipid of the pegylated lipid by a linker moiety selected from an ester-free linker moiety or an ester-containing linker moiety. Non-limiting examples of the ester-free linker moiety include amido (—C(O) NH—), amino (—NR—), carbonyl (—C(O)—), carbamate (—NHC (O)O—), urea (—NHC (O) NH—), disulfide (—S—S—), ether (—O—), succinyl (—(O) CCH2CH2C(O)—), succinamidyl (—NHC (O) CH2CH2C (O) NH—), ether, disulfide and combinations thereof. For example, the linker may contain a carbamate linker moiety and an amido linker moiety. Non-limiting examples of the ester-containing linker moiety include carbonate (—OC (O)O—), succinoyl, phosphate ester (—O—(O) POH—O—), sulfonate ester, and combinations thereof.

The PEG moiety of the pegylated lipid of the LNP of the disclosure described herein may have an average molecular weight ranging from about 550 daltons to about 10,000 daltons. In certain embodiments, the PEG moiety has an average molecular weight of from about 750 daltons to about 5,000 daltons, about 1,000 daltons to about 4,000 daltons, about 1,500 daltons to about 3,000 daltons, about 750 daltons to about 3,000 daltons, or about 1750 daltons to about 2,000 daltons.

In some embodiments, the conjugated lipid (e.g., pegylated lipid) comprises from about 1 mol % to about 60 mol %, from about 2 mol % to about 50 mol %, from about 5 mol % to about 40) mol %, or from about 5 mol % to about 20 mol % of the total lipid present in the LNPs and/or LNP compositions. In certain embodiments, the conjugated lipid comprises from about 0).5 mol % to about 3 mol % of the total lipid present in the particle.

In additional embodiments, the conjugated lipid (e.g., pegylated lipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

For the lipid in the lipid-PEG conjugate of the LNP of the disclosure, any lipid capable of binding to polyethyleneglycol may be used without limitation, and the phospholipid and/or cholesterol which are other elements of the LNP may be also used. In some embodiments, the lipid in the lipid-PEG conjugate may be ceramide, dimyristoylglycerol (DMG), succinoyl-diacylglycerol (s-DAG), distearoylphosphatidylcholine (DSPC), distearoylphosphatidylethanolamine (DSPE), or cholesterol, but not limited thereto.

In the lipid-PEG conjugate of the LNP of the disclosure, the PEG may be directly conjugated to the lipid or linked to the lipid via a linker moiety. Any linker moiety suitable for binding PEG to the lipid may be used, and for example, includes an ester-free linker moiety and an ester-containing linker moiety. The ester-free linker moiety includes not only amido (—C(O) NH—), amino (—NR—), carbonyl (—C(O)—), carbamate (—NHC (O)O—), urea (—NHC (O) NH—), disulfide (—S—S—), ether (—O—), succinyl (—(O) CCH2CH2C(O)—), succinamidyl (—NHC (O) CH2CH2C (O) NH—), ether, disulfide but also combinations thereof (for example, a linker containing both a carbamate linker moiety and an amido linker moiety), but not limited thereto. The ester-containing linker moiety includes for example, carbonate (—OC (O)O—), succinoyl, phosphate ester (—O—(O) POH—O—), sulfonate ester, and combinations thereof, but not limited thereto.

### Steroids

In some embodiments, the LNPs and LNP compositions of the present disclosure include at least one steroid or derivative thereof. In some embodiments, the steroid comprises cholesterol. In some embodiments, the LNPs and LNP compositions comprise a cholesterol derivative selected from cholestanol, cholestanone, cholestenone, coprostanol, cholesteryl-2′-hydroxyethyl ether, cholesteryl-4′-hydroxybutyl ether, and any combination of the foregoing.

In some embodiments, the steroid (e.g., cholesterol) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the steroid (e.g., cholesterol) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

### Additional Lipid

In some embodiments, the LNPs and LNP compositions of the present disclosure include at least one additional lipid. In some embodiments, the additional lipid is non-cationic lipid selected from an anionic lipid, a neutral lipid, or both. In some embodiments, the additional lipid comprises at least one phospholipid. In some embodiments, the phospholipid is selected from an anionic phospholipid, a neutral phospholipid, or both. The phospholipid of the elements of the LNPs and LNP compositions can play a role in covering and protecting a core of the LNP formed by interaction of the cationic lipid and nucleic acid in the LNP and may facilitate cell membrane permeation and endosomal escape during intracellular delivery of the nucleic acid by binding to the phospholipid bilayer of a target cell. A phospholipid which can promote fusion of the LNP to a cell may include without limitation, any of the phospholipids selected from the group described below.

In some embodiments, the LNPs and LNP compositions comprise at least one phospholipid selected from, but not limited to, dipalmitoyl-phosphatidylcholine (DPPC), distearoyl-phosphatidylcholine (DSPC), dioleoyl-phosphatidylethanolamine (DOPE), dioleoyl-phosphatidylcholine (DOPC), dioleoyl-phosphatidylglycerol (DOPG), palmitoyloleovl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dipalmitoyl-phosphatidylglycerol (DPPG), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleovl-phosphatidylethanolamine (SOPE), egg phosphatidylcholine (EPC), phosphatidylethanolamine (PE), 1,2-dioleoyl-sn-glycero-3-phosphoethanolamine, 1-palmitoyl-2-oleoyl-sn-glycero-3-phosphocholine (POPC), 1,2-dioleoyl-sn-glycero-3-[phospho-L-serine] (DOPS), 1,2-dioleoyl-sn-glycero-3-[phospho-L-serine], and any combination of the foregoing. In one example, the LNP comprising DOPE may be effective in mRNA delivery (excellent drug delivery efficacy).

In some embodiments, the additional lipid (e.g., phospholipid) of the LNP of the disclosure comprises from about 1 mol % to about 60) mol %, from about 2 mol % to about 50) mol %, from about 5 mol % to about 40) mol %, or from about 5 mol % to about 20 mol % of the total lipid present in the LNPs and/or LNP compositions. In other embodiments, the additional lipid (e.g., phospholipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

It will be appreciated that the total lipid present in the LNPs and/or LNP compositions comprises the combination of the cationic lipid or ionizable cationic lipid, the conjugated lipid, (e.g., pegylated lipid), the steroid (e.g., cholesterol), and the additional lipid (e.g., phospholipid).

The LNPs and/or LNP compositions may be prepared by dissolving the total lipids (or a portion thereof) in an organic solvent (e.g., ethanol) followed by mixing through a micromixer with the payload (e.g., nucleic acids of the systems) dissolved in an acidic buffer (e.g., pH 4). At this pH the cationic lipid is positively charged and interacts with the negatively-charged nucleic acid polymers. The resulting nanostructures containing the nucleic acids are then converted to neutral LNPs when dialyzed against a neutral buffer which may then be followed by removal of the organic solvent (e.g., ethanol) and exchange the LNPs into physiologically relevant buffer. The LNPs and/or LNP compositions thus formed have a distinct electron-dense nanostructured core where the cationic lipids are organized into inverted micelles around the encapsulated payload, as opposed to traditional bilayer liposomal structures. In another embodiment, the LNP may form a bleb-like structure with nucleic acids in aqueous pockets along the non-electron dense lipid core.

b. Lipid Nanoparticle Properties

In some embodiments, the LNPs and/or LNP compositions comprise from about 50 mol % to about 85 mol % of the cationic lipid or ionizable cationic lipid, from about 0.5 mol % to about 10 mol % of the conjugated lipid, (e.g., pegylated lipid), from about 0.5 mol % to about 10 mol % of the steroid (e.g., cholesterol) and from about 5 mol % to about 50 mol % of the additional lipid (e.g., phospholipid). In some embodiments, the LNPs and/or LNP compositions comprise from about 50 mol % to about 85 mol % of the cationic lipid or ionizable cationic lipid, from about 0.5 mol % to about 5 mol % of the conjugated lipid, (e.g., pegylated lipid), from about 0.5 mol % to about 5 mol % of the steroid (e.g., cholesterol) and from about 5 mol % to about 20 mol % of the additional lipid (e.g., phospholipid).

In some embodiments, the LNPs and/or LNP compositions of the disclosure comprise cationic lipid; additional lipid (e.g., phospholipid):steroid (e.g., cholesterol):conjugated lipid, (e.g., pegylated lipid) at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5.

In some embodiments, the LNPs and/or LNP compositions of the disclosure have a total lipid: payload ratio (mass/mass) of from about 1 to about 100. In some embodiments, the total lipid: payload ratio is about 1 to about 50, from about 2 to about 25, from about 3 to about 20, from about 4 to about 15, or from about 5 to about 10. In some embodiments, the total lipid: payload ratio is about 5 to about 15, e.g., about 5, 6, 7, 8, 9, 10, 11, 12, 13, 14, 15, or an intermediate range of any of the foregoing.

In certain embodiments, the LNPs of the disclosure comprise a total lipid:nucleic acid mass ratio of from about 5:1 to about 15:1. In some embodiments, the weight ratio of the cationic lipid and nucleic acid comprised in the LNP may be 1 to 20:1, 1 to 15:1, 1 to 10:1, 5 to 20:1, 5 to 15:1, 5 to 10:1, 7.5 to 20:1, 7.5 to 15:1, or 7.5 to 10:1.

In some embodiments, the LNP of the disclosure may comprise the cationic lipid of 20 to 50 parts by weight, the phospholipid of 10 to 30 parts by weight, cholesterol of 20 to 60 parts by weight (or 20 to 60 parts by weight), and lipid-PEG conjugate of 0.1 to 10 parts by weight (or 0.25 to 10 parts by weight, 0.5 to 5 parts by weight). Alternatively, the LNP may comprise the cationic lipid of 20 to 50% by weight, phospholipid of 10 to 30% by weight, cholesterol of 20 to 60% by weight (or 30 to 60% by weight), and lipid-PEG conjugate of 0.1 to 10% by weight (or 0.25 to 10% by weight, 0.5 to 5% by weight) based on the total nanoparticle weight. As a further alternative, the LNP may comprise the cationic lipid of 25 to 50% by weight, phospholipid of 10 to 20% by weight, cholesterol of 35 to 55% by weight, and lipid-PEG conjugate of 0.1 to 10% by weight (or 0.25 to 10% by weight, 0.5 to 5% by weight), based on the total nanoparticle weight.

In some embodiments, the LNPs of the present disclosure have a mean diameter of from about 20 to 200 nm, 20 to 180 nm, 20 to 170 nm, 20 to 150 nm, 20 to 120 nm, 20 to 100 nm, 20 to 90 nm, 30 to 200 nm, 30 to 180 nm, 30 to 170 nm, 30 to 150 nm, 30 to 120 nm, 30 to 100 nm, 30 to 90 nm, 40 to 200 nm, 40 to 180 nm, 40 to 170 nm, 40 to 150 nm, 40 to 120 nm, 40 to 100 nm, 40 to 90 nm, 40 to 80 nm, 40 to 70 nm, 50 to 200 nm, 50 to 180 nm, 50 to 170 nm, 50 to 150 nm, 50 to 120 nm, 50 to 100 nm, 50 to 90 nm, 60 to 200 nm, 60 to 180 nm, 60 to 170 nm, 60 to 150 nm, 60 to 120 nm, 60 to 100 nm, 60 to 90 nm, 70 to 200 nm, 70 to 180 nm, 70 to 170 nm, 70 to 150 nm, 70 to 120 nm, 70 to 100 nm, 70 to 90 nm, 80 to 200 nm, 80 to 180 nm, 80 to 170 nm, 80 to 150 nm, 80 to 120 nm, 80 to 100 nm, 80 to 90 nm, 90 to 200 nm, 90 to 180 nm, 90 to 170 nm, 90 to 150 nm, 90 to 120 nm, or 90 to 100 nm, or an intermediate range of any of the foregoing.

In some embodiments, the LNPs and/or LNP compositions of the disclosure have a positive charge at acidic pH and may encapsulate the payload (e.g., therapeutic agent) through electrostatic charges produced by negative charges of the payload (e.g., therapeutic agent). The term “encapsulation,” refers to the mixture of lipids surrounding and embedding the payload (e.g., therapeutic agent) at physiological conditions, forming the LNPs. The term “encapsulation efficiency,” as used herein is the amount of payload (e.g., therapeutic agent) encapsulated by the LNPs divided by the total amount of payload (e.g., therapeutic agent) used to load the payload (e.g., therapeutic agent) into the LNPs. The encapsulation efficiency of the LNPs and/or LNP compositions may be 70% or more, 75% or more, 80% or more, 85% or more, 90% or more, 91% or more, 92% or more, 94% or more, or 95% or more. In other embodiments, the encapsulation efficiency of the LNPs and/or LNP compositions is about 80% to 99%, about 85% to 98%, about 88% to 95%, about 90% to 95%, or the payload (e.g., nucleic acids of the systems) may be fully encapsulated within the lipid portion of the LNPs compositions, and thereby protected from enzymatic degradation. In some embodiments, the payload (e.g., therapeutic agent) is not substantially degraded after exposure of the LNPs and/or LNP compositions to a nuclease at 37° C. for at least about 20, 30, 45, or 60 minutes or at least about 2, 3, 4, 5, 6, 7, 8, 9, 10, 12, 14, 16, 18, 20, 22, 24, 26, 28, 30, 32, 34, or 36 hours. In some embodiments, the payload (e.g., nucleic acids of the systems) is complexed with the lipid portion of the LNPs and/or LNP compositions. The LNPs and/or LNP compositions of the present disclosure are non-toxic to mammals such as humans.

The term “fully encapsulated” indicates that the payload (e.g., the nucleic acids of the system) in the LNPs and/or LNP compositions is not significantly degraded after exposure to conditions that significantly degrade free DNA, RNA, or protein. In a fully encapsulated system, less than about 25%, more preferably less than about 10%, and most preferably less than about 5% of the payload (e.g., nucleic acids of the system) in the LNPs and/or LNP compositions is degraded by conditions that would degrade 100% of a non-encapsulated payload. “Fully encapsulated” also indicates that the LNPs and/or LNP compositions are serum-stable, and do not decompose into their component parts upon in vivo administration.

In some embodiments, the amount of the LNPs and/or LNP compositions having the payload (e.g., therapeutic agent), encapsulated therein is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the amount of the payload (e.g., the nucleic acids), encapsulated within the LNPs and/or LNP compositions is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the nucleic acids of the disclosure, such as the mRNA encoding the CasX fusion protein, and/or the gRNA, may be provided in a solution to be mixed with a lipid solution such that the nucleic acids may be encapsulated in the lipid nanoparticles. A suitable nucleic acid solution may be any aqueous solution containing the nucleic acid to be encapsulated at various concentrations. For example, a suitable nucleic acid solution may contain the nucleic acid (or nucleic acids) at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.25 mg/ml, 1.5 mg/ml, 1.75 mg/ml, or 2.0 mg/ml. In some embodiments, the nucleic acid comprises an mRNA encoding an CasX, and a suitable mRNA solution may contain the mRNA at a concentration ranging from about 0.01-2.0 mg/ml, 0.01-1.5 mg/ml, 0.01-1.25 mg/ml, 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml. In some embodiments, a suitable gRNA solution may contain an gRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0).3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml.

In some embodiments, the LNP may have an average diameter of 20 nm to 200 nm, 20 to 180 nm, 20 nm to 170 nm, 20 nm to 150 nm, 20 nm to 120 nm, 20 nm to 100 nm, 20 nm to 90 nm, 30 nm to 200 nm, 30 to 180 nm, 30 nm to 170 nm, 30 nm to 150 nm, 30 nm to 120 nm, 30 nm to 100 nm, 30 nm to 90 nm, 40 nm to 200 nm, 40 to 180 nm, 40 nm to 170 nm, 40 nm to 150 nm, 40 nm to 120 nm, 40 nm to 100 nm, 40 nm to 90 nm, 40 nm to 80 nm, 40 nm to 70 nm, 50 nm to 200 nm, 50 to 180 nm, 50 nm to 170 nm, 50 nm to 150 nm, 50 nm to 120 nm, 50 nm to 100 nm, 50 nm to 90 nm, 60 nm to 200 nm, 60 to 180 nm, 60 nm to 170 nm, 60 nm to 150 nm, 60 nm to 120 nm, 60 nm to 100 nm, 60 nm to 90 nm, 70 nm to 200 nm, 70 to 180 nm, 70 nm to 170 nm, 70 nm to 150 nm, 70 nm to 120 nm, 70 nm to 100 nm, 70 nm to 90 nm, 80 nm to 200 nm, 80 to 180 nm, 80 nm to 170 nm, 80 nm to 150 nm, 80 nm to 120 nm, 80 nm to 100 nm, 80 nm to 90 nm, 90 nm to 200 nm, 90 to 180 nm, 90 nm to 170 nm, 90 nm to 150 nm, 90 nm to 120 nm, or 90 nm to 100 nm for easy introduction into liver tissue, hepatocytes and/or LSEC (liver sinusoidal endothelial cells). The LNP may be sized for easy introduction into organs or tissues, including but not limited to liver, lung, heart, spleen, as well as to tumors. When the size of the LNP is smaller than the above range, it can be difficult to maintain stability as the surface area of the LNP is excessively increased, and thus delivery to the target tissue and/or drug effect may be reduced. The LNP may specifically target liver tissue. Without wishing to be bound by theory, it is thought that one mechanism by which LNP may be used to deliver therapeutic agents is through the imitation of the metabolic behaviors of natural lipoproteins, and so LNP may be usefully delivered to a subject through the lipid metabolism processes carried out by the liver. During the delivery of therapeutic agents to hepatocytes or and/or LSEC (liver sinusoidal endothelial cells), the diameter of the fenestrae leading from the sinusoidal lumen to the hepatocytes and LSEC is about 140 nm in mammals and about 100 nm in humans, so the LNP composition for therapeutic agent delivery having LNPs with a diameter in the above ranges may have excellent delivery efficiency to hepatocytes and LSEC when compared to LNP having the diameter outside the above range.

According to one example, the LNPs of the LNP composition may comprise the cationic lipid: phospholipid: cholesterol: lipid-PEG conjugate in the range described above or at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5. The LNP comprising components at a molar ratio in the above range may have excellent delivery efficiency of therapeutic agents specific to cells of target organs.

In certain aspects, the LNP exhibit a positive charge under the acidic pH condition by showing a pKa of 5 to 8, 5.5 to 7.5, 6 to 7, or 6.5 to 7, and may encapsulate a nucleic acid with high efficiency by easily forming a complex with a nucleic acid through electrostatic interaction with a therapeutic agent such as a nucleic acid showing a negative charge. In such cases, the LNP may be usefully used as a composition for intracellular or in vivo delivery of a therapeutic agent (for example, nucleic acid).

Herein, “encapsulate” or “encapsulation” refers to incorporation of a therapeutic agent efficient delivery, i.e., by surrounding it by the particle surface and/or embedding it within the particle interior. The encapsulation efficiency means the content of the therapeutic agent encapsulated in the LNP relative the total therapeutic agent content used for preparation of the LNP.

The encapsulation of the nucleic acids of the composition in the LNP may be 70% or more, 75% or more, 80% or more, 85% or more, 90% or more, 91% or more, 92% or more, 94% or more, or 95% or more of LNP in the composition encapsulate nucleic acids. In some embodiments, the encapsulation of the nucleic acids of the composition in the LNP is such that between 80% to 99%, between 80% to 97%, between 80% to 95%, between 85% to 95%, between 87% to 95%, between 90% to 95%, between 91% or more to 95% or less, 91% or more to 94% or less, over 91% to 95% or less, 92% to 99%, between 92% to 97%, or between 92% to 95% of the LNP in the composition encapsulate nucleic acids. In some embodiments, the mRNA encoding the CasX and a gRNA of any of the embodiments of the disclosure are fully encapsulated in the LNP.

The target organs to which a nucleic acid is delivered by the LNP include, but are not limited to the liver, lung, heart, spleen, as well as to tumors. The LNP according to one example is liver tissue-specific and has excellent biocompatibility and can deliver the nucleic acids of a composition with high efficiency, and thus it can be usefully used in related technical fields such as lipid nanoparticle-mediated gene therapy. In a particular embodiment, the target cell to which the nucleic acids are delivered by the LNP according to one example may be a hepatocyte and/or LSEC in vivo. In other embodiments, the disclosure provides LNP formulated for delivery of the nucleic acids of the embodiments to cells ex vivo.

The disclosure provides a pharmaceutical composition comprising a plurality of LNPs comprising nucleic acids, such as mRNA encoding an engineered CasX protein and/or a gRNA variant described herein, and a pharmaceutically acceptable carrier.

In certain embodiments, the LNP comprising the nucleic acid(s) has an electron dense core.

The disclosure provides LNP comprising one or more nucleic acids comprising: (a) an mRNA encoding the CasX, and/or a gRNA variant described herein; (b) one or more cationic lipids or ionizable cationic lipids or salts thereof comprising from about 50 mol % to about 85 mol % of the total lipid present in the LNP; (c) one or more non-cationic lipids comprising from about 13 mol % to about 49.5 mol % of the total lipid present in the LNP; and (d) one or more conjugated lipids that inhibit aggregation of LNPs comprising from about 0).5 mol % to about 2 mol % of the total lipid present in the particle. In another embodiment, the disclosure provides LNP comprising one or more nucleic acids comprising: (a) an mRNA encoding the engineered CasX protein, and/or a gRNA variant described herein; (b) one or more cationic lipids or ionizable cationic lipids or salts thereof comprising from about 22 mol % to about 85 mol % of the total lipid present in the LNP; (c) one or more non-cationic/phospholipids comprising from about 10 mol % to about 70 mol % of the total lipid present in the LNP; (d) 15 mol % to about 50 mol % sterol, and (d) 1 mol % to about 5 mol % lipid-PEG or lipid-PEG-peptide in the particle. In certain embodiments the CasX mRNA and gRNA may be present in the same nucleic acid-lipid particle, or they may be present in different nucleic acid-lipid particles.

The disclosure provides LNP comprising one or more nucleic acids comprising: (a) an mRNA encoding the engineered CasX protein described herein; (b) a cationic lipid or a salt thereof comprising from about 52 mol % to about 62 mol % of the total lipid present in the LNP; (c) a mixture of a phospholipid and cholesterol or a derivative thereof comprising from about 36 mol % to about 47 mol % of the total lipid present in the LNP; and (d) a PEG-lipid conjugate comprising from about 1 mol % to about 2 mol % of the total lipid present in the LNP. In particular embodiments, the formulation is a four-component system comprising about 1.4 mol % PEG-lipid conjugate (e.g., PEG2000-C-DMA), about 57.1 mol % cationic lipid (e.g., DLin-K-C2-DMA) or a salt thereof, about 7.1 mol % DPPC (or DSPC), and about 34.3 mol % cholesterol (or derivative thereof).

In other embodiments, the LNP comprising one or more nucleic acids comprises: (a) an mRNA encoding the engineered CasX protein and/or a gRNA of any of the embodiments described herein; (b) a cationic lipid or a salt thereof comprising from about 46.5 mol % to about 66.5 mol % of the total lipid present in the LNP; (c) cholesterol or a derivative thereof comprising from about 31.5 mol % to about 42.5 mol % of the total lipid present in the LNP; and (d) a PEG-lipid conjugate comprising from about 1 mol % to about 2 mol % of the total lipid present in the LNP. In particular embodiments, the formulation is a three-component system which is phospholipid-free and comprises about 1.5 mol % PEG-lipid conjugate (e.g., PEG2000-C-DMA), about 61.5 mol % cationic lipid (e.g., DLin-K-C2-DMA) or a salt thereof, and about 36.9 mol % cholesterol (or derivative thereof).

Additional formulations are described in PCT Publication No. WO 09/127060 and US patent publication numbers US 2011/0071208 A1 and US 2011/0076335 A1, the disclosures of which are herein incorporated by reference in their entirety.

In other embodiments, the LNP comprising one or more nucleic acids comprises: (a) an mRNA encoding the engineered CasX protein and/or a gRNA of any of the embodiments described herein; (b) one or more cationic lipid or ionizable cationic lipids or salts thereof comprising from about 2 mol % to about 50 mol % of the total lipid present in the LNP; (c) one or more non-cationic lipid or ionizable cationic lipids comprising from about 5 mol % to about 90 mol % of the total lipid present in the LNP; and (d) one or more conjugated lipids that inhibit aggregation of particles comprising from about 0.5 mol % to about 20 mol % of the total lipid present in the LNP.

In other embodiments, the LNP comprising one or more nucleic acids comprises: (a) an mRNA encoding the engineered CasX protein and a gRNA of any of the embodiments described herein; (b) a cationic lipid or a salt thereof comprising from about 30 mol % to about 50 mol % of the total lipid present in the LNP; (c) a mixture of a phospholipid and cholesterol or a derivative thereof comprising from about 47 mol % to about 69 mol % of the total lipid present in the LNP; and (d) a PEG-lipid conjugate comprising from about 1 mol % to about 3 mol % of the total lipid present in the LNP. In particular embodiments, the formulation is a four-component system which comprises about 2 mol % PEG-lipid conjugate (e.g., PEG2000-C-DMA), about 40 mol % cationic lipid (e.g., DLin-K-C2-DMA) or a salt thereof, about 10 mol % DPPC (or DSPC), and about 48 mol % cholesterol (or derivative thereof).

In other embodiments, the LNP comprising one or more nucleic acids comprises: (a) an mRNA encoding the engineered CasX protein and a gRNA of any of the embodiments described herein; (b) one or more cationic lipid or ionizable cationic lipids or salts thereof comprising from about 50 mol % to about 65 mol % of the total lipid present in the LNP; (c) one or more non-cationic lipid or ionizable cationic lipids comprising from about 25 mol % to about 45 mol % of the total lipid present in the LNP; and (d) one or more conjugated lipids that inhibit aggregation of particles comprising from about 5 mol % to about 10 mol % of the total lipid present in the LNP.

In other embodiments, the LNP comprising one or more nucleic acids comprises: (a) an mRNA encoding the engineered CasX protein and a gRNA of any of the embodiments described herein; (b) a cationic lipid or a salt thereof comprising from about 50 mol % to about 60 mol % of the total lipid present in the LNP; (c) a mixture of a phospholipid and cholesterol or a derivative thereof comprising from about 35 mol % to about 45 mol % of the total lipid present in the LNP; and (d) a PEG-lipid conjugate comprising from about 5 mol % to about 10 mol % of the total lipid present in the LNP.

In certain embodiments, the non-cationic lipid mixture in the formulation comprises: (i) a phospholipid of from about 10 mol % to about 70 mol % of the total lipid present in the LNP; (ii) cholesterol or a derivative thereof of from about 15 mol % to about 50 mol % of the total lipid present in the LNP; and 1-5% lipid-PEG or lipid-PEG-peptide. In particular embodiments, the formulation is a four-component system which comprises about 7 mol % PEG-lipid conjugate (e.g., PEG750-C-DMA), about 54 mol % cationic lipid (e.g., DLin-K-C2-DMA) or a salt thereof, about 7 mol % DPPC (or DSPC), and about 32 mol % cholesterol (or derivative thereof).

In other embodiments, the LNP comprising one or more nucleic acids comprises: (a) an mRNA encoding the engineered CasX protein and/or a gRNA of any of the embodiments described herein; (b) a cationic lipid or a salt thereof comprising from about 55 mol % to about 65 mol % of the total lipid present in the LNP; (c) cholesterol or a derivative thereof comprising from about 30 mol % to about 40 mol % of the total lipid present in the LNP; and (d) a PEG-lipid conjugate comprising from about 5 mol % to about 10 mol % of the total lipid present in the LNP. In particular embodiments, the formulation is a three-component system which is phospholipid-free and comprises about 7 mol % PEG-lipid conjugate (e.g., PEG750)—C-DMA), about 58 mol % cationic lipid (e.g., DLin-K-C2-DMA) or a salt thereof, and about 35 mol % cholesterol (or derivative thereof).

In other embodiments, the LNP comprising one or more nucleic acids comprises: (a) an mRNA encoding the engineered CasX protein and/or a gRNA of any of the embodiments described herein; (b) a cationic lipid or a salt thereof comprising from about 48 mol % to about 62 mol % of the total lipid present in the LNP; (c) a mixture of a phospholipid and cholesterol or a derivative thereof, wherein the phospholipid comprises about 7 mol % to about 17 mol % of the total lipid present in the LNP, and wherein the cholesterol or derivative thereof comprises about 25 mol % to about 40 mol % of the total lipid present in the LNP; and (d) a PEG-lipid conjugate comprising from about 0.5 mol % to about 3.0 mol % of the total lipid present in the LNP.

In some embodiments, the CasX:gRNA systems provided herein for a method of modification of an LPA target nucleic acid in a population of cells comprise a sequence encoding a an engineered CasX protein comprising SEQ ID NOS: 19834-19837, or a variant sequence at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gRNA scaffold comprises SEQ ID NOS: 464-541 or a sequence at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gRNA comprises a targeting sequence complementary to an LPA target nucleic acid. In a particular embodiment, wherein the systems are formulated in LNP, the CasX is encoded by an mRNA sequence selected from the group consisting of SEQ ID NOS: 19834-19837, and the gRNA scaffold comprises a sequence of SEQ ID NOS: 464-541, and comprises a targeting sequence complementary to an LPA target nucleic acid. In some embodiments of the method, the cells to be modified are selected from the group consisting of rodent cells, mouse cells, rat cells, and non-human primate cells. In other embodiments of the method, the cells to be modified are human cells. In some embodiments of the method, the modification of the population of cells occurs in vivo in a subject, wherein the subject is selected from the group consisting of a rodent, a mouse, a rat, a non-human primate, and a human.

In some LNP-mediated delivery embodiments, the method of treating an LPA-related disease or disorder in a subject comprises pretreating the subject with a therapeutic agent that increases hepatic LDL receptor (LDLR) expression. In some embodiments, the therapeutic agent is a PCSK9 inhibitor, such as a monoclonal antibody, nucleic acid-based agent, or a small molecule. Exemplary therapeutic agents include, but are not limited to, evolocumab, inclisiran, alirocumab, and MK-0616. Without wishing to be bound by theory or mechanism, it is believed that the pretreatment with an inhibitor of PCSK9, may lead to an increase in hepatic LDL receptor (LDLR) expression that, in turn, may facilitate the uptake of an LNP comprising the CasX:gRNA composition that is subsequently administered to the subject. By increasing the hepatic cell uptake of the LNP, it is expected that editing of the LPA gene will be enhanced such that an improvement in the LPA-related disorder would be attained.

### VIII. Compositions, Pharmaceutical Compositions, Kits, and Articles of Manufacture for Medicaments

In some embodiments, the disclosure provides compositions comprising: i) an engineered CasX protein and a gRNA of any of the embodiments of the disclosure comprising a targeting sequence specific for an LPA gene; ii) one or more nucleic acids encoding the engineered CasX protein and the gRNA of (i); iii) an LNP or synthetic nanoparticle comprising a gRNA and an mRNA encoding the engineered CasX protein.

In some embodiments, the disclosure provides pharmaceutical compositions comprising: i) an engineered CasX protein and a gRNA of any of the embodiments of the disclosure comprising a targeting sequence specific for an LPA gene; ii) one or more nucleic acids encoding the engineered CasX protein and the gRNA of (i); iii) an LNP or synthetic nanoparticle comprising a gRNA and an mRNA encoding an engineered CasX protein, together with one or more pharmaceutically suitable excipients. In some embodiments, the pharmaceutical composition is formulated for a route of administration selected from the group consisting of intravenous, intraportal vein injection, intraperitoneal, intramuscular, subcutaneous, intraocular, and oral routes. In one embodiment, the pharmaceutical composition is in a liquid form or a frozen form. In another embodiment, the pharmaceutical composition is in a pre-filled syringe for a single injection. In another embodiment, the pharmaceutical composition is in solid form, for example the pharmaceutical composition is lyophilized.

Excipients can include a salt, an isotonic agent, a serum protein, a buffer or other pH-controlling agent, an anti-oxidant, a thickener, an uncharged polymer, a preservative or a cryoprotectant. Excipients used in compositions of the disclosure may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. In some embodiments the isotonic agent may be glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples of preservatives include polyhexamethylene-biguanidine, benzalkonium chloride, stabilized oxychloro complexes (such as those known as Purite R), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

Typically, a pharmaceutical formulation is formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring. Additional pharmaceutical formulations appropriate for administration are applicable in the methods and compositions disclosed herein (see. e.g., Remington's Pharmaceutical Sciences (1990) 18th ed., Mack Publishing Co., Easton, Pa.; Remington's Pharmaceutical Sciences (2023) 23rd ed., Elsevier Publishing; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993).

In other embodiments, provided herein are kits comprising an engineered CasX protein and one or a plurality of the gRNAs of any of the embodiments of the disclosure. The gRNAs comprise a targeting sequence specific for an LPA gene target nucleic acid sequence. In some embodiments, the kits comprise a suitable container (for example a tube, vial or plate). In exemplary embodiments, the engineered CasX protein comprises a sequence of any one of SEQ ID NOS: 19699-19702 or 19830-19837. In exemplary embodiments, the gRNA comprises a sequence of any one of SEQ ID NOS: 464-466, or chemically-modified versions of such gRNA, including a sequence selected from the group consisting of SEQ ID NOS: 19855-19862.

In other embodiments, provided herein are kits comprising an LNP formulation encapsulating an mRNA encoding an engineered CasX protein and one or a plurality of gRNA of any of the embodiments of the disclosure. In some embodiments, the kits comprise suitable container (for example a tube, vial or plate). In an exemplary embodiment, the kit comprises the engineered CasX protein comprising a sequence of any one of SEQ ID NOS: 19699-19702 or 19830-19837, and the gRNA comprises a scaffold of any one of SEQ ID NOS: 464-541. In some embodiments of the kit, the gRNA comprises a sequence selected from the group consisting of SEQ ID NOS: 19855-19862. In some embodiments, the kit comprises a first LNP formulation encapsulating the mRNA, and a second LNP formulation encapsulating the gRNA. In some embodiments of the kit, the mRNA and gRNA are encapsulated in a single LNP formulation.

In some embodiments, the kit comprises a gRNA or a vector encoding a gRNA, wherein the gRNA comprises a scaffold sequence selected from the group consisting of SEQ ID NOS: 464-541, and a targeting sequence complementary to an LPA gene target nucleic acid to be modified.

In certain embodiments, provided herein are kits comprising an engineered CasX protein and gRNA, the kit comprising an engineered CasX protein comprising a sequence of SEQ ID NOS: 19699-19702 or 19830-19837, and the gRNA comprising a scaffold comprising a sequence of SEQ ID NOS: 464-541 and a targeting sequence complementary to an LPA target nucleic acid to be modified. In some embodiments, the gRNA is a chemically-modified comprises a sequence selected from the group consisting of SEQ ID NOS: 19855-19862.

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modification applications, and instructions for use.

In some embodiments, the kit comprises a vector comprising a sequence encoding an engineered CasX protein of the disclosure and a CasX gRNA of the disclosure.

In some embodiments, the disclosure provides compositions for use in the treatment a subject having an LPA-related disease. In some embodiments, the disclosure provides compositions for use in the manufacture of a medicament for the treatment a subject having an LPA-related disease. In some embodiments, the composition comprises an engineered CasX protein of any of the embodiments disclosed herein. In some embodiments, the composition comprises a gRNA of any of the embodiments disclosed herein. In some embodiments, the composition comprises a nucleic acid encoding an engineered CasX protein of any of the embodiments disclosed herein. In some embodiments, the composition comprises an mRNA encoding an engineered CasX protein of any of the embodiments disclosed herein. In some embodiments, the composition comprises a gene editing pair of an engineered CasX protein and a gRNA. In some embodiments, the composition comprises a vector of any of the embodiments disclosed herein. In some embodiments, the composition comprises an LNP of any of the embodiments disclosed herein. In some embodiments, the composition comprises a combination of the foregoing.

### IX. Therapeutic Methods

In another aspect, the present disclosure relates to methods of treating a LPA-related disease or disorder in a subject in need thereof, using the systems of the disclosure.

The present disclosure provides methods of treating an LPA-related disorder in a subject in need thereof, including but not limited to cardiovascular disease, metabolic disease, hyperglycemia, prediabetes, diabetes (type I and type II), obesity, insulin resistance, metabolic syndrome and diabetic dyslipidemia, or inflammatory disease. In certain embodiments, the subject has Lp (a) levels ≥70 nmol/L, ≥80 nmol/L, ≥90 nmol/L, ≥100 nmol/L, ≥110 nmol/L, ≥120 nmol/L, ≥130 nmol/L, ≥140 nmol/L, ≥150 nmol/L, ≥160) nmol/L, ≥170) nmol/L, ≥175 nmol/L, ≥180 nmol/L, ≥190 nmol/L, ≥200 nmol/L. ≥250) nmol/L. ≥300) nmol/L. ≥350) nmol/L, or ≥400 nmol/L. In some embodiments of the method of treatment, the subject is selected from the group consisting of a rodent, a mouse, a rat, and a non-human primate. In other embodiments of the method of treatment, the subject is a human.

In certain embodiments, the methods of the disclosure are utilized in the treatment of cardiovascular diseases, disorders or conditions (CVD) including, but not limited to one or more of coronary artery disease, coronary heart disease (CHD), acute myocardial infarction (AMI), chronic CHD, cardiovascular events, arterial hypertension (HT), aortic stenosis (e.g., calcific aortic valve stenosis), aneurysm (e.g., abdominal aortic aneurysm), angina, arrhythmia, atherosclerosis, cerebrovascular disease, cerebrovascular stroke dyslipidemia, hypercholesterolemia, hyperlipidemia, hypertension, hypertriglyceridemia, myocardial infarction, peripheral vascular disease (e.g., peripheral artery disease), stroke, ischemia, elevated lipoprotein (a) levels, elevated apoprotein (a) levels, or combinations thereof.

In certain embodiments, the methods of the disclosure are utilized in the treatment of inflammatory diseases, disorders or conditions including, but not limited to, elevated Lp (a) associated CVD risk, recurrent cardiovascular events with elevated Lp (a), aortic stenosis (e.g., calcific aortic valve stenosis associated with high Lp (a)), coronary artery disease (CAD), Alzheimer's Disease and thromboembolic diseases such as, but not limited to, stroke, thrombosis, myocardial infarction and peripheral vascular disease.

A number of therapeutic strategies have been used to design the compositions for use in the methods of treatment of a subject with an LPA-related disease. Additionally, the methods can be used to treat a subject in advance of any symptom of disease. As described herein, the methods of treatment can prevent, treat and/or ameliorate an LPA-related disease of a subject. In some embodiments, the disclosure provides a method of treating an LPA-related disease in a subject in need thereof, comprising administering a therapeutically effective dose of i) a composition comprising an engineered CasX protein and a gRNA of any of the embodiments described herein; ii) an LNP comprising a gRNA and an mRNA encoding a CasX of any of the embodiments described herein; iii) one or more nucleic acids encoding or comprising the compositions of (i) or (ii); iv) a vector selected from the group consisting of a retroviral vector, a lentiviral vector, an adenoviral vector, an adeno-associated viral (AAV) vector, a herpes simplex virus (HSV) vector and comprising the nucleic acids of (iii); or v) combinations of two or more of (i)-(iv), wherein the LPA gene of the cells is modified by the CasX protein. In some embodiments, the disclosure provides a method of treating an LPA-related disease in a subject in need thereof, comprising administering a therapeutically effective dose of an LNP comprising a gRNA and an mRNA encoding a CasX to the subject, wherein the LPA gene of the cells is modified by the CasX protein. In some embodiments of the method of treatment, the modification comprises introducing a single-stranded break in the LPA target nucleic acid sequence. In some embodiments of the method of treatment, the modification comprises introducing a double-stranded break in the LPA target nucleic acid sequence. In some embodiments of the method of treatment, expression of the LPA gene is reduced or eliminated in the modified cells of the subject.

In some embodiments of the method of treatment, the method comprises administering to the subject one or more therapeutically effective doses of a system of the disclosure, wherein the contacting of the cells of the subject with the engineered CasX and the gRNA results in modification of the target nucleic acid of the cells by the components of the CasX:gRNA system.

In some embodiments of the method of treatment, the method comprises administering to the subject one or more therapeutically effective doses of an mRNA comprising a sequence encoding the engineered CasX of the disclosure, including the sequences of SEQ ID NOS: 4-7 as set forth in Table 1. In some embodiments of the method of treatment, the method comprises administering to the subject one or more therapeutically effective doses of an mRNA comprising a sequence selected from the group consisting of 19830-19841, or a sequence having at least about 70%, at least about 80%, at least about 90%, at least about 95%, at least about 96%, at least about 97%, at least about 98%, at least about 99% sequence identity thereto. In some embodiments of the method of treatment, the method comprises administering to the subject one or more therapeutically effective doses of an mRNA comprising a sequence selected from the group consisting of 19830-19841.

In some embodiments of the method of treatment, the method comprises administering to the subject one or more therapeutically effective doses of a gRNA embodiment of the disclosure, including the sequences of SEQ ID NOS: 464-466 of Table 13, with a linked targeting sequence complementary to a target nucleic acid of the LPA gene. In some embodiments of the method, the targeting sequence of the gRNA is selected from the group consisting of SEQ ID NOS: 749-903 and 13210-19698. In some embodiments of the method, the targeting sequence of the gRNA comprises a sequence selected from the group consisting of SEQ ID NO: 817, SEQ ID NO: 826, SEQ ID NO: 898, and SEQ ID NO: 901. In some embodiments of the method, a targeting sequence for linkage to a gRNA scaffold of the disclosure, e.g., gRNA 174, 235, or 316, or a chemically-modified version thereof, may comprise the sequence of SEQ ID NO: 901. In some embodiments of the method, the method comprises administering to the subject one or more therapeutically effective doses of a chemically modified gRNA with a targeting sequence comprising a sequence selected from the group consisting of SEQ ID NOS: 19855-19862, or a sequence having at least about 70%, at least about 80%, at least about 90%, at least about 95%, at least about 96%, at least about 97%, at least about 98%, at least about 99% sequence identity thereto. In some embodiments, the chemically-modified gRNA targeting an LPA target sequence comprises a sequence selected from the group consisting of SEQ ID NOS: 19855-19862. In some embodiments, the chemically-modified gRNA targeting an LPA target sequence comprises a sequence of SEQ ID NO: 19855. In some embodiments, the chemically-modified gRNA targeting an LPA target sequence comprises a sequence of SEQ ID NO: 19856. In some embodiments, the chemically-modified gRNA targeting an LPA target sequence comprises a sequence of SEQ ID NO: 19857. In some embodiments, the chemically-modified gRNA targeting an LPA target sequence comprises a sequence of SEQ ID NO: 19858. In some embodiments, the chemically-modified gRNA targeting an LPA target sequence comprises a sequence of SEQ ID NO: 19859. In some embodiments, the chemically-modified gRNA targeting an LPA target sequence comprises a sequence of SEQ ID NO: 19860. In some embodiments, the chemically-modified gRNA targeting an LPA target sequence comprises a sequence of SEQ ID NO: 19861. In some embodiments, the chemically-modified gRNA targeting an LPA target sequence for comprises a sequence of SEQ ID NO: 19862.

In some embodiments of the method of treatment, the method comprises administering to the subject one or more therapeutically effective doses of an mRNA comprising a sequence selected from the group consisting of 19830-19841, or a sequence having at least about 70%, at least about 80%, at least about 90%, at least about 95%, at least about 96%, at least about 97%, at least about 98%, at least about 99% sequence identity thereto and a chemically modified gRNA with a targeting sequence comprising a sequence selected from the group consisting of SEQ ID NOS: 19855-19862, or a sequence having at least about 70%, at least about 80%, at least about 90%, at least about 95%, at least about 96%, at least about 97%, at least about 98%, at least about 99% sequence identity thereto. In some embodiments of the method of treatment, the method comprises administering to the subject one or more therapeutically effective doses of an mRNA comprising a sequence selected from the group consisting of 19830-19841 and a chemically modified gRNA with a targeting sequence comprising a sequence selected from the group consisting of SEQ ID NOS: 19855-19862.

In some embodiments of the method of treatment, the method comprises administering to the subject one or more therapeutically effective doses of an LNP comprising a gRNA and an mRNA encoding the engineered CasX protein according to a treatment regimen, wherein the contacting of the cells of the subject with the LNP results in modification of the target nucleic acid of the cells by the components of the CasX:gRNA system. In the foregoing embodiment, the gRNA scaffold is selected from the group consisting of variant 174 (SEQ ID NO: 464), 235 (SEQ ID NO: 465), and 316 (SEQ ID NO: 464), or chemically modified versions thereof, the mRNA encodes an engineered CasX protein selected from the group consisting of 491 (SEQ ID NO: 4), 515 (SEQ ID NO: 5), 676 (SEQ ID NO: 6), and 812 (SEQ ID NO: 7), the targeting sequence of the gRNA is selected from the group consisting of SEQ ID NOS: 749-903 and 13210-19698. The CasX:gRNA components are encapsidated either singly or in combination in an LNP formulation of an embodiment described herein. In some embodiments, the engineered CasX is encoded by an mRNA comprising a sequence selected from the group consisting of SEQ ID NOS: 19830-19837, or a sequence having at least about 70%, at least about 80%, at least about 90%, at least about 95%, at least about 96%, at least about 97%, at least about 98%, at least about 99% sequence identity thereto. In some embodiments, the engineered CasX is encoded by an mRNA comprising a sequence selected from the group consisting of SEQ ID NOS: 19830-19837. In some embodiments, the engineered CasX is encoded by an mRNA comprising a sequence of SEQ ID NO: 19830. In some embodiments, the engineered CasX is encoded by an mRNA comprising a sequence of SEQ ID NO: 19831. In some embodiments, the engineered CasX is encoded by an mRNA comprising a sequence of SEQ ID NO: 19832. In some embodiments, the engineered CasX is encoded by an mRNA comprising a sequence of SEQ ID NO: 19833. In some embodiments, the engineered CasX is encoded by an mRNA comprising a sequence of SEQ ID NO: 19834. In some embodiments, the engineered CasX is encoded by an mRNA comprising a sequence of SEQ ID NO: 19835. In some embodiments, the engineered CasX is encoded by an mRNA comprising a sequence of SEQ ID NO: 19836. In some embodiments, the engineered CasX is encoded by an mRNA comprising a sequence of SEQ ID NO: 19837. In some embodiments, the chemically-modified gRNA targeting LPA for use in the systems of the disclosure comprises a sequence selected from the group consisting of SEQ ID NOS: 19855-19862, or a sequence having at least about 70%, at least about 80%, at least about 90%, at least about 95%, at least about 96%, at least about 97%, at least about 98%, at least about 99% sequence identity thereto. In some embodiments, the chemically-modified gRNA targeting LPA for use in the systems of the disclosure comprises a sequence selected from the group consisting of SEQ ID NOS: 19855-19862. In some embodiments, the chemically-modified gRNA comprises the sequence of SEQ ID NO: 19855. In some embodiments, the chemically-modified gRNA comprises the sequence of SEQ ID NO: 19856. In some embodiments, the chemically-modified gRNA comprises the sequence of SEQ ID NO: 19857. In some embodiments, the chemically-modified gRNA comprises the sequence of SEQ ID NO: 19858. In some embodiments, the chemically-modified gRNA comprises the sequence of SEQ ID NO: 19859. In some embodiments, the chemically-modified gRNA comprises the sequence of SEQ ID NO: 19860. In some embodiments, the chemically-modified gRNA comprises the sequence of SEQ ID NO: 19861. In some embodiments, the chemically-modified gRNA comprises the sequence of SEQ ID NO: 19862.

In some embodiments of the method of treatment, the subject is selected from the group consisting of a rodent, a mouse, a rat, and a non-human primate. In some embodiments of the method, the subject is a human. In some embodiments of the method of treatment, the cell of the subject for modification is a hepatocyte, or a cell of the intestine, the kidney, the central nervous system, a smooth muscle cell, a macrophage, a cell of the retina, or a cell of arterial walls such as the endothelium.

In some embodiments, the method of treatment of the subject results in improvements in quantifiable markers or clinically-relevant endpoints of the disease. Methods of obtaining samples from treated subjects for analysis to determine the effectiveness of the treatment, such as body fluids or tissues, and methods of preparation of the samples to allow for analysis are well known to those skilled in the art. Methods for analysis of RNA and protein levels are discussed above and are well known to those skilled in the art. For example, glucose levels or insulin resistance can be measured and quantified by standard tests known in the art. Other metabolic markers include, but are not limited to cytokine levels associated with inflammatory disease, Lp (a), triglycerides, apo (a), apo (b), very low-density lipoproteins (VLDL), VLDL-remnants, intermediate-density lipoproteins (IDL), low-density lipoproteins (LDL) or cholesterol levels. For such markers, in certain embodiments, the method of treatment results in a decrease in one or more markers by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, or a range defined by any two of these values. In a particular embodiment, the method of treatment results in reduction of Lp (a) expression by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, or a range defined by any two of these values. In certain embodiments, Lp (a) expression is reduced to at least about ≥400 nmol/L, at least about ≥3800 nmol/L, at least about ≥360 nmol/L, at least about ≥350 nmol/L, at least about ≥340 nmol/L, at least about ≥320 nmol/L, at least about ≥300 nmol/L, at least about ≥280 nmol/L, at least about ≥260 nmol/L, at least about ≥240 nmol/L, at least about ≥220) nmol/L, at least about ≥200 nmol/L, at least about ≥180 nmol/L, at least about ≥160 nmol/L, at least about ≥140 nmol/L, at least about ≥120 nmol/L, at least about ≥110 nmol/L, at least about ≥100 nmol/L, at least about ≥90 nmol/L, at least about ≥80 nmol/L, at least about ≥70 nmol/L, or at least about ≥60 nmol/L.

In some embodiments, the method of treatment results in improvement in at least one clinical endpoint selected from the group consisting of change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina, visual acuity, peripheral arterial disease, oxidized phospholipid levels, reduction in aortic valve stenosis, and reduction of thrombosis.

In some embodiments, the method of treatment results in improvement in at least two clinically-relevant endpoints selected from the group consisting of change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina, visual acuity, peripheral arterial disease, oxidized phospholipid levels, reduction in aortic valve stenosis, reduction of thrombosis.

In some embodiments, the method results in improvement in at least two clinically-relevant endpoints selected from the group consisting of change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina, visual acuity, peripheral arterial disease, oxidized phospholipid levels, reduction in aortic valve stenosis, or reduction of thrombosis.

The following Examples are merely illustrative and are not meant to limit any aspects of the present disclosure in any way.

### EXAMPLES

### Example 1: Identification of CasX Spacers Targeting the Human LPA Locus

This example describes the methods and criteria used to identify spacers for targeting the human LPA locus using CasX. Briefly, a pipeline algorithm was designed and used to determine all possible spacer sequences (also referred to herein as “targeting sequences”) with NTC PAMs throughout the human LPA locus, which were then computationally filtered to define an initial group of TTC spacers for experimental screening and assessment.

### Materials and Methods

To identify potential spacers throughout the human LPA locus, the reference transcript NM_005577.4 (Ensembl ID ENST00000316300.10) was aligned with the human reference genome build hg38 (GRCh38) to extract the full sequence of the human LPA locus. The target search region used to determine all potential spacers was defined as starting at 10 KB upstream of the transcription start site (TSS) through 5 KB downstream of the transcription stop site. Spacers were determined based on the availability of NTC PAMs; consequently, a total of 19,155 NTC spacers (RNA sequences provided in SEQ ID NO: 544-19698), which are 20 base pairs in length, were identified throughout the target LPA locus. These spacers were then functionally annotated by overlaying key genomic features based on their positioning, i.e., determining whether the putative spacer targeted an exon, an intron, or a candidate cis-regulatory element (cCRE), was proximal to the transcriptional start site (“TSS-proximal”), and/or overlapped with a common site of genetic variation (e.g., SNPs). Furthermore, a sequence conservation analysis was performed, such that each nucleotide within an edit window for each spacer sequence was evaluated and scored to determine the extent of evolutionary conservation at the individual bases across 100 model organisms (Pollard K S et al., Detection of nonneutral substitution rates on mammalian phylogenies. Genome Res. 20 (1): 110-121 (2010); Kent W J et al., The human genome browser at UCSC. Genome Res. 12 (6): 996-1006 (2002)). The edit window of a spacer was defined as the region of the target DNA that would be subjected to indel formation following CasX-gRNA cleavage. The resulting PhyloP score, which was determined by calculating the mean of conservation scores for each base within the edit window, is believed to provide an indication of the likelihood of the spacer to perturb the target gene function.

To narrow down and determine an initial group of spacers for experimental screening and assessment, the extracted spacers were subjected to a set of filtering criteria. Firstly, non-specific spacers were excluded by performing a computational prediction analysis of off-target sites for each gRNA spacer. Briefly, off-target sites with NTC PAMs were identified and categorized based on the number of mismatched bases from the on-target sequence, and spacers with one or more off-target sites within one mismatch of the on-target sequence were excluded. Next, to select spacers that are expected to achieve a functional knockdown of the LPA mRNA and secreted Lp (a) levels, spacers that met one or more of the following inclusion criteria were selected for inclusion: spacers that were functionally annotated to target an exon or a cCRE of the human LPA locus, spacers that were evolutionarily conserved with a mean PhyloP score >0.8, and any additional spacers that were TSS-proximal. This resulted in a filtered set of 360 NTC spacers (sequences listed in Table 14). Lastly, among the filtered set of 360 NTC spacers, TTC spacers that did not overlap with a SNP having a minor allele frequency (MAF) of >0.05 were selected for experimental screening and evaluation. As a result, a total of 142 TTC spacers were identified, and the sequences and predicted edit windows of these spacers are listed in Table 15. In experiments involving use of lentivirus (Examples 2, 3 and 4), the DNA versions of the listed spacers of Table 15 were created by substituting thymine for uracil in the sequences.

TABLE 14
RNA sequences of 360 NTC spacers
targeting the human LPA locus

| | | SEQ |
|---|---|---|
| | | ID |
| PAM | Spacer sequence (RNA) | NO: |
| ATC | AGAUUUAGCACAAUGGCAAA | 544 |
| ATC | UCAAGAUUUAGCACAAUGGC | 545 |
| ATC | UCAUUUGUAUCCGUAGUCUG | 546 |
| ATC | AAUGGCCAGAAGGUCAUAUG | 547 |
| ATC | AUGGACUGAUCCUGCCGCUG | 548 |
| ATC | UGCCGCUGGACUUACCCUGC | 549 |
| ATC | GGGAGGGCUUCAAUCCUCUU | 550 |
| ATC | UCUUGUCACCUGUGGCCUAC | 551 |
| ATC | CUGAGGGCAGGUAGGCCACA | 552 |
| ATC | AGUGGCCGACAUAGAGAUGU | 553 |
| ATC | CUAUGUCGGCCACUGGAUGG | 554 |
| ATC | ACUUUCCUGGCAACUUGCGG | 555 |
| ATC | UGAUAAUGGUGAAAACCUAC | 556 |
| ATC | GCUAUUUGAGCAAGGCAAAC | 557 |
| ATC | CCUGGACUACAUAGUUGUGU | 558 |
| ATC | GGCAGAGGGUGCAUCACCUG | 559 |
| ATC | GGGCAGAGCUGCAUGAUGGA | 560 |
| ATC | UGCAGCUCUGCCCAGAUUGA | 561 |
| ATC | UUCCAGGAGGGGGUGGUGGC | 562 |
| ATC | AUCCUUCCAGGAGGGGGUGG | 563 |
| ATC | UAUACCAAACUGUGCCGAAA | 564 |
| ATC | CCACAUCCUAUACCAAACUG | 565 |
| ATC | UAAAAGUAGCUCAUAUUUGC | 566 |
| ATC | UUCCUGAUUAGUCUUGUUUC | 567 |
| ATC | GGAAAGAUGAAGGUCUAGGG | 568 |
| ATC | AUUGACAUUGCACUCUCAAA | 569 |
| ATC | UAGUUUCCAUGGGCUGAAAU | 570 |
| ATC | ACAUCCUAGUUUCCAUGGGC | 571 |
| ATC | UUCCAAUGUGUGCUGGAAUG | 572 |
| ATC | UCUAUUCACAAAUUGCUAAG | 573 |
| ATC | GCCAAUGCCUGUUCAAUAAU | 574 |
| ATC | AGUUUUCCAGCAUAGAGCAU | 575 |
| ATC | UCCUCCUUGCCCUGCCCUCA | 576 |
| ATC | AGUAGAAAGGCAGAUGAACA | 577 |
| ATC | GCCUUUCUACUGGAUACUUG | 578 |
| ATC | GCAUACAAACACAUGUUCUC | 579 |
| ATC | GCAUGUGGCCCCCACCACAC | 580 |
| ATC | UGGACCACAUGGCUUUGCUC | 581 |
| ATC | AUGACACCACAUCAACAUAA | 582 |
| ATC | ACAUAAUAGGACCACAGAAA | 583 |
| ATC | UGUGGCAGCCCCUUAUUGUU | 584 |
| ATC | GGGUAAUAGUUGGAGGCGCG | 585 |
| ATC | AGGCACAUACUUCAUUACUG | 586 |
| ATC | GGAUUUCGGCAGUAGUUCUU | 587 |
| ATC | AGAACUACUGCCGAAAUCCA | 588 |
| ATC | UGUGGCAGCCCCUUGGUGUU | 589 |
| ATC | GAGCAUCGUGUCAGGUUGCA | 590 |
| ATC | AGUGUCCUUGCAACUCUCAC | 591 |
| ATC | GGGACCACCGUGAGAGUUGC | 592 |
| ATC | AAGCACAGAGGCUUCUUCUG | 593 |
| ATC | UGGACCCCGGGGCUUUGCUC | 594 |
| ATC | GAGGACAACAGAAUAUUAUC | 595 |
| ATC | AGAUGCUGAGAUUAGUCCUU | 596 |
| ATC | AUGGUAUAACACCAAGGACU | 597 |
| ATC | AGUGUCCUUGCGACGUCCAC | 598 |
| ATC | CUGGACAUUGCGUCAGGUUG | 599 |
| ATC | ACUCUCCUCACAACUCCCAC | 600 |
| ATC | CCUCGGUAGCAGUCCUGGAC | 601 |
| ATC | AGGCACACUCUCCACCACUA | 602 |
| ATC | UCCGAUGCCAAUGUGGUGUC | 603 |
| ATC | GAGGAUCCCAUUAUACUAUC | 604 |
| ATC | CAUUAUACUAUCCAAAUGCG | 605 |
| ATC | AGUGUCCUCACAACUCCCAC | 606 |
| ATC | UGGACCACAGGGCUUUUCUC | 607 |
| ATC | AGGCAUAUCCUCCACCACUG | 608 |
| ATC | UCCACCACUGUCACAGGAAG | 609 |
| ATC | UAGAUGACCAAGAUUGACAG | 610 |
| ATC | UGGUCAUCUAUGAUACCACA | 611 |
| ATC | AUGAUACCACACUGGCAUCA | 612 |
| ATC | GAGUUGUCGGGGAAGUGGAG | 613 |
| ATC | GAGCUCUAGUGCUGUGUUGG | 614 |
| ATC | UCUGAAACCAGGACCCACUU | 615 |
| ATC | AUGCUAUUUAAGAUACUCAG | 616 |
| ATC | UAAAUAGCAUGGAUGGAGCU | 617 |
| ATC | CAUGUAAAUGAACUCAUACA | 618 |
| ATC | UUUACAUUUCCUCCAAAAGA | 619 |
| ATC | GUGCUACCCCCUUACCACUG | 620 |
| ATC | GGAUUCCUGCAGUAGUUCUC | 621 |
| ATC | GACACAAUGCUCAGAAACAG | 622 |
| ATC | GGUGUCCUAGAGACUCCCAC | 623 |
| ATC | GGCCACAGACUUCUUACCUG | 624 |
| ATC | AGGUUCCAAGCCUAGGGCCU | 625 |
| ATC | CACGCUAGACAAAGAAGGGU | 626 |
| ATC | UAGGAAAUCAAUGAAGGGUA | 627 |
| ATC | AUGAAGGGUAGUGCAGCUCU | 628 |
| ATC | UACGAGAAAGGCCAUCUGGG | 629 |
| ATC | AAUGUCAAUUCCCCUUCCCC | 630 |
| ATC | UCAUCUUUUCUAGGUCCUCA | 631 |
| ATC | CAUGUUCAGGAAAUAGAAGU | 632 |
| ATC | GCUUGUGUGGGCUCCAAGAA | 633 |
| ATC | CCAGACUACAUGGUCACCGC | 634 |
| ATC | AUGUAAGUCUCAUCUGCAAU | 635 |
| ATC | GCCCAGUGAUUGCAGAUGAG | 636 |
| ATC | GCAAUCACUGGGCUGAUCCC | 637 |
| ATC | CUGGGCUGAUCCCGUGGCCC | 638 |
| ATC | CGUGGCCCUGUCUCCAGGGC | 639 |
| ATC | UCAGGUUACCUGUGGCCCAC | 640 |
| ATC | CUACAUUGGCCACGAGAUGG | 641 |
| ATC | UGAUAAGAGGGAAAACCCAC | 642 |
| ATC | CCUGGACUGCACAGAUGUGU | 643 |
| ATC | GUGCAGUCCAGGUGAUGCAU | 644 |
| ATC | CAUGCCCAAUGCUCGGUAGU | 645 |
| ATC | UUGUCUUCGGUGUCAGAAAA | 646 |
| ATC | AAGUUUCCAAUGUUGCAAGC | 647 |
| ATC | CUAUAAGUAUAUUUGUGCUG | 648 |
| ATC | AUUGAUCUUUUCUUACCUGG | 649 |
| ATC | ACCUACUUAGAAGCUGAAAC | 650 |
| ATC | UUACCCACGUUUCAGCUUCU | 651 |
| CTC | AUGUCGGCCACUGGAUGGCA | 652 |
| CTC | CCCCUAGACCUUCAUCUUUC | 653 |
| CTC | UUCCCUCACCCCUAGACCUU | 654 |
| CTC | ACAUUGCCUUUAUGUCUCCU | 655 |
| CTC | GCUGCUCCACAUUGCCUUUA | 656 |
| CTC | AACUAUUACCCCGAUUCCAA | 657 |
| CTC | AGGCUUGGAAUCGGGGUAAU | 658 |
| CTC | CACCUGACACUGGGAUCUGU | 659 |
| CTC | GAUGCAGAAUGGACUGCCUU | 660 |
| CTC | GAAUGUUAUUCUGGCUCCAA | 661 |
| CTC | AGGCUUGGAGCCAGAAUAAC | 662 |
| CTC | GUCCAUAAUGGUAGUAGCAG | 663 |
| CTC | GUAACUCUGUCCAUAAUGGU | 664 |
| CTC | CGGUGGUCCCAGAUCCAAGC | 665 |
| CTC | AGGUUGUACCCAUUUGGAUA | 666 |
| CTC | UGGGAGAAAACUCAAAGACA | 667 |
| CTC | CCUCACAACUCCCACGGUGG | 668 |
| CTC | UCACAACUCCCACGGUGGUC | 669 |
| CTC | CAACUCCCACGGUGGUCCCA | 670 |
| CTC | GUAGCAGUCCUGGACCCCAG | 671 |
| CTC | GUCCAUCACCUCGGUAGCAG | 672 |
| CTC | AUAACUCUGUCCAUCACCUC | 673 |
| CTC | CCACCACUAUCACAGGAAGA | 674 |
| CTC | AUUCUGUCACUGGACAUCGU | 675 |
| CTC | CAACUCCCACAGUGGCCCCG | 676 |
| CTC | CACAGUGGCCCCGGUUCCAA | 677 |
| CTC | GAAACAGAAUCAGGUGUCCU | 678 |
| CTC | GCUUGAUUUUGUCUAUUUUC | 679 |
| CTC | CACCUGAUGCUGGGGUCCAU | 680 |
| CTC | GACACAGAAGGGACUGUGGU | 681 |
| CTC | CUGCUUGAAGAAGUACGUUU | 682 |
| CTC | AGGCCUUCAUCCUACAAGGU | 683 |
| CTC | AAUCUCAUGUUCAGGAAAUA | 684 |
| CTC | ACAUUGGCCACGAGAUGGCA | 685 |
| CTC | ACUGCUCUGUUCUUCUGGAA | 686 |
| CTC | AGGAAGCCCAGCUCCUUGUU | 687 |
| CTC | AUAACAAGGAGCUGGGCUUC | 688 |
| CTC | UUGUUAUUGAGAAUGAAGUG | 689 |
| CTC | GCACAAAUAUACUUAUAGUG | 690 |
| CTC | GCACUUAUUUUGAUUUGAAU | 691 |
| GTC | GCGAGUAGGUGCUGCCAUCC | 692 |
| GTC | AUGCCUCCAGCCAAAUGCCC | 693 |
| GTC | AGGGGUGAGGGAAGGAGUAA | 694 |
| GTC | AUAGAUGCUGGGAAGUGGUG | 695 |
| GTC | UAUAAAAUAUUUGAGAGUGC | 696 |
| GTC | UCACGGGGAAAUUAAACUUA | 697 |
| GTC | UUAGUAUCACUGCAAACCUC | 698 |
| GTC | UCUAUGACACCACAUCAACA | 699 |
| GTC | UAUUAUGUUGAUGUGGUGUC | 700 |
| GTC | CGCCUCCAACUAUUACCCCG | 701 |
| GTC | AUUCUGCAUCUGAGCAUCGU | 702 |
| GTC | CUCCGAAUGUUAUUCUGGCU | 703 |
| GTC | GGCACAACUUCUUACCUUGU | 704 |
| GTC | UCUGUUUUAAUUUCAGCACU | 705 |
| GTC | UGUACCCCGGGGGUUUCCUC | 706 |
| GTC | AUAAUGGUAGUAGCAGUCCU | 707 |
| GTC | GACUAUGCUGGUGUGGUGUC | 708 |
| GTC | CCAGGCAUUGUGUCAGGUUG | 709 |
| GTC | UUGCAACUCUCACGGUGGUC | 710 |
| GTC | CAGAUCCAAGCACAGAGGCU | 711 |
| GTC | GGCCAUAGACUUCCUACCUU | 712 |
| GTC | UUGAAGAAAACUCAAGGUUG | 713 |
| GTC | GUCUUUGAAGAAAACUCAAG | 714 |
| GTC | UUGGUGUUAUACCAUGGAUC | 715 |
| GTC | CAAGGACACUUGAUUCUGUC | 716 |
| GTC | UUGCGACGUCCACGGCUGUU | 717 |
| GTC | ACGGCUGUUUCUGAACAAGG | 718 |
| GTC | AGUGAUGGAAUCAACUCUCC | 719 |
| GTC | CAGUUCCAAGCACAGAGCUU | 720 |
| GTC | GACUGCAGGCUUCUUACCUU | 721 |
| GTC | UGGACCCCAGUGCUGUUUUC | 722 |
| GTC | AUCACCUCGGUAGCAGUCCU | 723 |
| GTC | AGGACUGCUACCGAGGUGAU | 724 |
| GTC | AUGACACCACAUUGGCAUCG | 725 |
| GTC | UCACAACUCCCACAGUGGCC | 726 |
| GTC | AUCUUGGUCAUCUAUGAUAC | 727 |
| GTC | CUAGGACACCUGAUUCUGUU | 728 |
| GTC | AUUUUCAGCACCAACUGAGC | 729 |
| GTC | UCCAGGUUCCAAGCCUAGGG | 730 |
| GTC | CUGGAAUGAACGUGCUGUGU | 731 |
| GTC | CUGGUGCUACACAAUGAAUC | 732 |
| GTC | GCACCCACUCUGGGGAUAUU | 733 |
| GTC | GUUUUCCCUUAAACGUACUU | 734 |
| GTC | GUGAUGACGGCAGGCCUGUA | 735 |
| GTC | GGGGAUGGCAGACAAGCUGG | 736 |
| GTC | GCCAUCCCCAGACUACAUGG | 737 |
| GTC | UGGCGGUGACCAUGUAGUCU | 738 |
| GTC | CCGCCAGGACUGAAUGUUAC | 739 |
| GTC | AUGAGUAUGUGCUGCCAUCU | 740 |
| GTC | GUGCCUCUGGCCAAAUGCUC | 741 |
| GTC | AUUAUUUCCAGCAUGCUAAA | 742 |
| GTC | UCGUUUGAUUGCUGUCUAUU | 743 |
| GTC | GACCUUAAAAGCUUAUACAC | 744 |
| GTC | GUCAGACCUUAAAAGCUUAU | 745 |
| GTC | GACUGACAAAUUCUGUAUUA | 746 |
| GTC | UAGCUAUGACACCUUAAUAC | 747 |
| GTC | UAGCUAUGACAUUUGUUAAA | 748 |
| TTC | CGGGGAAAUUAAACUUAUUU | 749 |
| TTC | CAGUGAUCUACAUCCUAGUU | 750 |
| TTC | GAAGGAAGCUCUGUGCUUGG | 751 |
| TTC | CCGUGAAGACUGUCACGGUG | 752 |
| TTC | AGCACACAUUGGAAAGAUAA | 753 |
| TTC | AAUGUGUGCUGGAAUGCCCG | 754 |
| TTC | AGGGUGAUGGGCUUCACAAC | 755 |
| TTC | AAGCACAGAGCUUCCUUCUG | 756 |
| TTC | CUCCACUUCCCCGACAACUC | 757 |
| TTC | CCGACAACUCAGAUGACUCA | 758 |
| TTC | UCAGAAGGAAGCUCUGUGCU | 759 |
| TTC | UACCUUCUUCAGAAGGAAGC | 760 |
| TTC | AUUUCCUGAACAUGAGAUUC | 761 |
| TTC | GACUACGGAUACAAAUGAAG | 762 |
| TTC | CAGGGCCAAGGCCUUGUACA | 763 |
| TTC | UAGUGGGGCAUGUUAGCAAA | 764 |
| TTC | ACAGCAAAACUAUUUUGCUA | 765 |
| TTC | AUCCUCUUGUCACCUGUGGC | 766 |
| TTC | CCAUUAUCAGGAUGUUUGCC | 767 |
| TTC | UUCUCAAUCACAGGGAGCUG | 768 |
| TTC | CUUAUGUUGUCAUUUCGGCA | 769 |
| TTC | UCUUUCCUGAUUAGUCUUGU | 770 |
| TTC | UAUCCCCUGCACUCCCUACA | 771 |
| TTC | GCCCAUGGAAACUAGGAUGU | 772 |
| TTC | CAAAUUGCUAAGUCUUUUAC | 773 |
| TTC | AUAAUCCAGUUUUCCAGCAU | 774 |
| TTC | UGCAAAGUCCUUGGCCACAG | 775 |
| TTC | UCUGCCUUUCUACUGGAUAC | 776 |
| TTC | GAACCUCCCCUGACCCUGUA | 777 |
| TTC | UUACUGUCACAGGAAGAACC | 778 |
| TTC | GCACUGACUGAGGAAACCCC | 779 |
| TTC | GAAACAGCCGUGGACGUCGC | 780 |
| TTC | GCACCAACUGAAAACAGCAC | 781 |
| TTC | GCACCACCUGAGAAAAGCCC | 782 |
| TTC | CCCCCACACCAUACAGAAGC | 783 |
| TTC | AGUGUACCAGUAGGUUGCAA | 784 |
| TTC | GCACUGAGUAUCUUAAAUAG | 785 |
| TTC | UUUACAUGAGAUCAUUUACA | 786 |
| TTC | GAAGGAGGCCCUAGGCUUGG | 787 |
| TTC | UUCCAGGGACAAAUAAAUGG | 788 |
| TTC | UUGUGUAGCACCAGGGACCA | 789 |
| TTC | UGCAUCCCACGCUAGACAAA | 790 |
| TTC | UCAUAAUGACUCUAAAUUUG | 791 |
| TTC | AGCAGUGAGCAGCAGUCAGC | 792 |
| TTC | GUCCUGGCGGUGACCAUGUA | 793 |
| TTC | UUCUCAAUAACAAGGAGCUG | 794 |
| TTC | CUCUGUCUCCCGUCCAAUUA | 795 |
| TTC | GCUUCUAAGUAGGUUGAUGC | 796 |
| TTC | AAUCAAAAUAAGUGCAGAGU | 797 |
| TTC | AACAAGGGUUCUUAACAAGA | 798 |
| TTC | UGGCAACUUGCGGUAGGUUU | 799 |
| TTC | AUCAUGCAGCUCUGCCCAGA | 800 |
| TTC | AGGAGGGGGUGGUGGCUCAU | 801 |
| TTC | UGAUUAGUCUUGUUUCCUCC | 802 |
| TTO | CCCCUCAGCUGCUCCACAUU | 803 |
| TTC | CAGCAUCUAUUGACAUUGCA | 804 |
| TTC | CAGUUCAAAAUCUUGUAUAU | 805 |
| TTC | AGCACCGUGACAGUCUUCAC | 806 |
| TTC | AGCAUAGAGCAUAUUAAAUU | 807 |
| TTC | UCAAUUUAAUAUGCUCUAUG | 808 |
| TTC | CUAGAAGUCUCGCUGCUUUC | 809 |
| TTC | AUGGAAUUAAGCAACAGGAC | 810 |
| TTC | AUGGAAGCUUGGCUAUCUUC | 811 |
| TTC | CCCAACAUGGCAGAACUCCG | 812 |
| TTC | GUGGUAGCACUCCUGCACCC | 813 |
| TTC | UGUGACAGUAAUGAAGUAUG | 814 |
| TTC | UACCUUCUUCAGAAGAAGCC | 815 |
| TTC | AUCACUGGACAUUGCGUCAG | 816 |
| TTC | UGUGAUAGUGGUGGAGAGUG | 817 |
| TTC | UUUUAUGGCUAACAUGAUAG | 818 |
| TTC | AAGCACAGAGGCUCCUUCUG | 819 |
| TTC | UGUGACAGUGGUGGAGGAUA | 820 |
| TTC | CCCCACCUGGAAACAGACUU | 821 |
| TTC | UCUUUUGGAGGAAAUGUAAA | 822 |
| TTC | UCCAAAAGAGGAAAAACUAA | 823 |
| TTC | UGCAGUAGUUCUCGGUCAGG | 824 |
| TTC | AAGCCUAGGGCCUCCUUCUG | 825 |
| TTC | AGGGACAAAUAAAUGGGCAG | 826 |
| TTC | AGACCUGCCCAUUUAUUUGU | 827 |
| TTC | UCAUUGCUCUGAGACUCUUA | 828 |
| TTC | CCUUCCCCUGAUCCAAGCCU | 829 |
| TTC | CUUAAACGUACUUCUUCAAG | 830 |
| TTC | UGAACAUGAGAUUCGAGGUU | 831 |
| TTC | UUACAGGCCUGCCGUCAUCA | 832 |
| TTC | CUCUUAUCAGGAUGUUUGCC | 833 |
| TTC | GCAGAGCUGCAUGGAGGAAG | 834 |
| TTC | UCCAUGCAGCUCUGCGGAAG | 835 |
| TTO | CCUACCUCUGUUUAGGGUGA | 836 |
| TTC | CUCAAUCCAAGUAACAAACC | 837 |
| TTC | CAGCUACCAGCUAUGCCAAA | 838 |
| TTC | GCACAGUUUGGUAUAGGAUG | 839 |
| TTC | GCAGUAGUUCUUGAUCAAGC | 840 |
| TTC | GAGGGACGAAGGCAGUCCAU | 841 |
| TTC | UCCCUCCGAAUGUUAUUCUG | 842 |
| TTC | GUGUCAGAAAACUGACACCG | 843 |
| TTC | GUGUCAGUUUUCUGACACCG | 844 |
| TTC | UUUGAUUGCUGUCUAUUAUU | 845 |
| TTC | GUCAUUUGAGUUAGCUCAGU | 846 |
| TTC | UAACAAGACUGAGCUAACUC | 847 |
| TTC | GGCCAUUUGAUUUAUUGGAG | 848 |
| TTC | UCUUUGGAGGUUUUCACAGG | 849 |
| TTC | UUGGAGGUUUUCACAGGGCC | 850 |
| TTC | CUAGAAUCUGCUAUUUGAGC | 851 |
| TTC | AGAGAACGGUGCUCCCUCAC | 852 |
| TTC | GACUACCAAGCAUCGGGCAG | 853 |
| TTC | GCCCAUUCAUUCCAGCACCG | 854 |
| TTC | AGCCUUUGGGUGUAGCCUGC | 855 |
| TTC | AUUCACAAAUUGCUAAGUCU | 856 |
| TTC | ACUGGAUACUUGUCAUCGGC | 857 |
| TTC | CUGCAGUGUGUCAUCUUUCA | 858 |
| TTC | AGGGAAUACAGGGUCAGGGG | 859 |
| TTC | CUCCUGCUGCCACCCCCACU | 860 |
| TTC | AGUCACUCUUUAACCCACUC | 861 |
| TTC | UCUGUUUUAAUUUUAGCAGC | 862 |
| TTC | GUUUUAAUUUUAGCAGCACC | 863 |
| TTC | GUGGUCCUAUUAUGUUGAUG | 864 |
| TTC | GCCAUGUUGGGGGAAGCCUC | 865 |
| TTC | GGUGCAACUUGGGUGGGCUU | 866 |
| TTC | AAAGACAAAGCCCACCCAAG | 867 |
| TTC | GGCCACAGGCUCCUUACCUU | 868 |
| TTC | UUUCUUUUCAUUUCAGCACC | 869 |
| TTC | UUUCAUUUCAGCACCAACUG | 870 |
| TTC | UCCUGUGACAGUAAUGAAGU | 871 |
| TTC | UUUGGAAUUUCCAGUGGCUU | 872 |
| TTC | GCAUCUGAGCAUCGUGUCAG | 873 |
| TTC | GGCUCCAAGCCUAGAGGCUU | 874 |
| TTC | GUUUUAAUUUCAGCACUGAC | 875 |
| TTC | GGGGUCCGACUAUGCUGGUG | 876 |
| TTC | GUCACCAGGCAUUGUGUCAG | 877 |
| TTC | UCAGAAGAAGCCUCUGUGCU | 878 |
| TTC | GUUGUCCUCUGAUGCCAGUG | 879 |
| TTC | UACCUUGUUCAGAAACAGCC | 880 |
| TTC | CUGAUGCCAGUGUGGUGUCA | 881 |
| TTC | GUGGUUCUCUGAUGCCAGUG | 882 |
| TTC | CUUGGGAGAAAACUCAAAGA | 883 |
| TTC | UCCUGUGAUAGUGGUGGAGA | 884 |
| TTC | GUAUGGUGUGGGGGUGAAGU | 885 |
| TTC | GAAACCAGGACCCACUUGAG | 886 |
| TTC | UAUUAUCUUCUGAAACCAGG | 887 |
| TTC | GUAUGAGUUCAUUUACAUGA | 888 |
| TTC | GUUGAAAGAAACCAAUGUAU | 889 |
| TTC | UGGAUUCAUUGUGUAGCACC | 890 |
| TTC | UUGUCUAGCGUGGGAUGCAU | 891 |
| TTC | CAGCUGCAGAAGAGGACAGU | 892 |
| TTC | UCUCAGCUGCAGAAGAGGAC | 893 |
| TTC | CGUAAGAUUACUACUCAUGA | 894 |
| TTC | UUUCCCACCUUGUGCCACAG | 895 |
| TTC | UUCUAGGUUUGGAAAGCACU | 896 |
| TTC | UCAAGCAGUGAGCAGCAGUC | 897 |
| TTC | UGGAGCCCACACAAGCAGAU | 898 |
| TTC | CAAAAUAUUUUGCUGAAAGU | 899 |
| TTC | GACACCGAAGACAAUGAUCU | 900 |
| TTC | CAAUAACAAGGAGCUGGGCU | 901 |
| TTC | AAGUAGGUUGAUGCUUCACU | 902 |
| TTC | GUAUUAAGGUGUCAUAGCUA | 903 |

TABLE 15
RNA sequences and edit window of 142 TTC spacers targeting the human
LPA locus

| | | | Edit window in human |
|---|---|---|---|
| | | SEQ | reference genome build |
| Spacer ID | Spacer sequence (RNA) | ID NO: | hg38 |
| TG-48-029 | GACUACGGAUACAAAUGAAG | 762 | cCRE |
| TG-48-038 | CAGGGCCAAGGCCUUGUACA | 763 | cCRE |
| TG-48-039 | UAGUGGGGCAUGUUAGCAAA | 764 | cCRE |
| TG-48-040 | ACAGCAAAACUAUUUUGCUA | 765 | cCRE |
| TG-48-018 | AUCCUCUUGUCACCUGUGGC | 766 | cCRE |
| TG-48-052 | CCAUUAUCAGGAUGUUUGCC | 767 | cCRE |
| TG-48-059 | UUCUCAAUCACAGGGAGCUG | 768 | — |
| TG-48-064 | CUUAUGUUGUCAUUUCGGCA | 769 | cCRE |
| TG-48-071 | UCUUUCCUGAUUAGUCUUGU | 770 | — |
| TG-48-009 | UAUCCCCUGCACUCCCUACA | 771 | Intron 1 |
| TG-48-008 | GCCCAUGGAAACUAGGAUGU | 772 | Intron 1 |
| TG-48-098 | CAAAUUGCUAAGUCUUUUAC | 773 | Intron 1 |
| TG-48-100 | AUAAUCCAGUUUUCCAGCAU | 774 | Intron 1 |
| TG-48-104 | UGCAAAGUCCUUGGCCACAG | 775 | Intron 1 |
| TG-48-107 | UCUGCCUUUCUACUGGAUAC | 776 | Intron 1 |
| TG-48-112 | GAACCUCCCCUGACCCUGUA | 777 | Intron 1 |
| TG-48-143 | UUACUGUCACAGGAAGAACC | 778 | Exon 18 |
| TG-48-163 | GCACUGACUGAGGAAACCCC | 779 | Exon 20 |
| TG-48-193 | GAAACAGCCGUGGACGUCGC | 780 | Exon 23 |
| TG-48-213 | GCACCAACUGAAAACAGCAC | 781 | Exon 26 |
| TG-48-236 | GCACCACCUGAGAAAAGCCC | 782 | Exon 28 |
| TG-48-250 | CCCCCACACCAUACAGAAGC | 783 | Intron 28 |
| TG-48-255 | AGUGUACCAGUAGGUUGCAA | 784 | Intron 28 |
| TG-48-256 | GCACUGAGUAUCUUAAAUAG | 785 | Intron 28 |
| TG-48-261 | UUUACAUGAGAUCAUUUACA | 786 | Intron 28 |
| TG-48-280 | GAAGGAGGCCCUAGGCUUGG | 787 | Exon 31 |
| TG-48-283 | UUCCAGGGACAAAUAAAUGG | 788 | Exon 32 |
| TG-48-286 | UUGUGUAGCACCAGGGACCA | 789 | Exon 33 |
| TG-48-290 | UGCAUCCCACGCUAGACAAA | 790 | Intron 33 |
| TG-48-292 | UCAUAAUGACUCUAAAUUUG | 791 | Intron 33 |
| TG-48-305 | AGCAGUGAGCAGCAGUCAGC | 792 | Exon 35 |
| TG-48-324 | GUCCUGGCGGUGACCAUGUA | 793 | Exon 37 |
| TG-48-352 | UUCUCAAUAACAAGGAGCUG | 794 | Exon 38 |
| TG-48-360 | CUCUGUCUCCCGUCCAAUUA | 795 | Exon 39, 3UTR |
| TG-48-362 | GCUUCUAAGUAGGUUGAUGC | 796 | Exon 39, 3UTR |
| TG-48-375 | AAUCAAAAUAAGUGCAGAGU | 797 | Exon 39, 3UTR |
| TG-48-033 | AACAAGGGUUCUUAACAAGA | 798 | cCRE |
| TG-48-017 | UGGCAACUUGCGGUAGGUUU | 799 | cCRE |
| TG-48-014 | AUCAUGCAGCUCUGCCCAGA | 800 | cCRE |
| TG-48-061 | AGGAGGGGGUGGUGGCUCAU | 801 | cCRE |
| TG-48-069 | UGAUUAGUCUUGUUUCCUCC | 802 | — |
| TG-48-078 | CCCCUCAGCUGCUCCACAUU | 803 | — |
| TG-48-080 | CAGCAUCUAUUGACAUUGCA | 804 | — |
| TG-48-083 | CAGUUCAAAAUCUUGUAUAU | 805 | Intron 1 |
| TG-48-087 | AGCACCGUGACAGUCUUCAC | 806 | Intron 1 |
| TG-48-102 | AGCAUAGAGCAUAUUAAAUU | 807 | Intron 1 |
| TG-48-103 | UCAAUUUAAUAUGCUCUAUG | 808 | Intron 1 |
| TG-48-007 | CUAGAAGUCUCGCUGCUUUC | 809 | Intron 1 |
| TG-48-006 | AUGGAAUUAAGCAACAGGAC | 810 | Intron 1 |
| TG-48-005 | AUGGAAGCUUGGCUAUCUUC | 811 | Intron 1 |
| TG-48-126 | CCCAACAUGGCAGAACUCCG | 812 | Intron 7 |
| TG-48-139 | GUGGUAGCACUCCUGCACCC | 813 | Exon 18 |
| TG-48-141 | UGUGACAGUAAUGAAGUAUG | 814 | Exon 18 |
| TG-48-179 | UACCUUCUUCAGAAGAAGCC | 815 | Exon 21 |
| TG-48-201 | AUCACUGGACAUUGCGUCAG | 816 | Exon 25 |
| TG-48-222 | UGUGAUAGUGGUGGAGAGUG | 817 | Exon 26 |
| TG-48-229 | UUUUAUGGCUAACAUGAUAG | 818 | Intron 26 |
| TG-48-235 | AAGCACAGAGGCUCCUUCUG | 819 | Exon 27 |
| TG-48-239 | UGUGACAGUGGUGGAGGAUA | 820 | Exon 28 |
| TG-48-245 | CCCCACCUGGAAACAGACUU | 821 | Intron 28 |
| TG-48-262 | UCUUUUGGAGGAAAUGUAAA | 822 | Intron 28 |
| TG-48-264 | UCCAAAAGAGGAAAAACUAA | 823 | Intron 28 |
| TG-48-267 | UGCAGUAGUUCUCGGUCAGG | 824 | Exon 29 |
| TG-48-281 | AAGCCUAGGGCCUCCUUCUG | 825 | Exon 31 |
| TG-48-284 | AGGGACAAAUAAAUGGGCAG | 826 | Exon 32 |
| TG-48-285 | AGACCUGCCCAUUUAUUUGU | 827 | Exon 32 |
| TG-48-299 | UCAUUGCUCUGAGACUCUUA | 828 | Intron 33 |
| TG-48-300 | CCUUCCCCUGAUCCAAGCCU | 829 | Intron 33 |
| TG-48-308 | CUUAAACGUACUUCUUCAAG | 830 | Exon 35 |
| TG-48-312 | UGAACAUGAGAUUCGAGGUU | 831 | Exon 36 |
| TG-48-318 | UUACAGGCCUGCCGUCAUCA | 832 | Exon 37 |
| TG-48-336 | CUCUUAUCAGGAUGUUUGCC | 833 | Intron 37 |
| TG-48-341 | GCAGAGCUGCAUGGAGGAAG | 834 | Intron 37 |
| TG-48-342 | UCCAUGCAGCUCUGCGGAAG | 835 | Intron 37 |
| TG-48-358 | CCUACCUCUGUUUAGGGUGA | 836 | Exon 39, Intron 38 |
| TG-48-359 | CUCAAUCCAAGUAACAAACC | 837 | Exon 39 |
| TG-48-368 | CAGCUACCAGCUAUGCCAAA | 838 | Exon 39, 3UTR |
| TG-48-066 | GCACAGUUUGGUAUAGGAUG | 839 | cCRE |
| TG-48-145 | GCAGUAGUUCUUGAUCAAGC | 840 | Exon 19 |
| TG-48-154 | GAGGGACGAAGGCAGUCCAU | 841 | Exon 19 |
| TG-48-155 | UCCCUCCGAAUGUUAUUCUG | 842 | Exon 19 |
| TG-48-343 | GUGUCAGAAAACUGACACCG | 843 | Intron 37 |
| TG-48-344 | GUGUCAGUUUUCUGACACCG | 844 | Intron 37 |
| TG-48-366 | UUUGAUUGCUGUCUAUUAUU | 845 | Exon 39, 3UTR |
| TG-48-031 | GUCAUUUGAGUUAGCUCAGU | 846 | cCRE |
| TG-48-032 | UAACAAGACUGAGCUAACUC | 847 | cCRE |
| TG-48-035 | GGCCAUUUGAUUUAUUGGAG | 848 | cCRE |
| TG-48-036 | UCUUUGGAGGUUUUCACAGG | 849 | cCRE |
| TG-48-037 | UUGGAGGUUUUCACAGGGCC | 850 | cCRE |
| TG-48-016 | CUAGAAUCUGCUAUUUGAGC | 851 | cCRE |
| TG-48-015 | AGAGAACGGUGCUCCCUCAC | 852 | cCRE |
| TG-48-056 | GACUACCAAGCAUCGGGCAG | 853 | cCRE |
| TG-48-088 | GCCCAUUCAUUCCAGCACCG | 854 | Intron 1 |
| TG-48-092 | AGCCUUUGGGUGUAGCCUGC | 855 | Intron 1 |
| TG-48-097 | AUUCACAAAUUGCUAAGUCU | 856 | Intron 1 |
| TG-48-109 | ACUGGAUACUUGUCAUCGGC | 857 | Intron 1 |
| TG-48-111 | CUGCAGUGUGUCAUCUUUCA | 858 | Intron 1 |
| TG-48-113 | AGGGAAUACAGGGUCAGGGG | 859 | Intron 1 |
| TG-48-114 | CUCCUGCUGCCACCCCCACU | 860 | Intron 1 |
| TG-48-115 | AGUCACUCUUUAACCCACUC | 861 | Intron 1 |
| TG-48-118 | UCUGUUUUAAUUUUAGCAGC | 862 | Exon 2, Intron 1 |
| TG-48-119 | GUUUUAAUUUUAGCAGCACC | 863 | Exon 2, Intron 1 |
| TG-48-124 | GUGGUCCUAUUAUGUUGAUG | 864 | Exon 2 |
| TG-48-127 | GCCAUGUUGGGGGAAGCCUC | 865 | Intron 7 |
| TG-48-128 | GGUGCAACUUGGGUGGGCUU | 866 | Intron 7 |
| TG-48-129 | AAAGACAAAGCCCACCCAAG | 867 | Intron 7 |
| TG-48-136 | GGCCACAGGCUCCUUACCUU | 868 | Exon 17, Intron 17 |
| TG-48-137 | UUUCUUUUCAUUUCAGCACC | 869 | Exon 18, Intron 17 |
| TG-48-138 | UUUCAUUUCAGCACCAACUG | 870 | Exon 18 |
| TG-48-142 | UCCUGUGACAGUAAUGAAGU | 871 | Exon 18 |
| TG-48-144 | UUUGGAAUUUCCAGUGGCUU | 872 | Exon 19, Intron 18 |
| TG-48-151 | GCAUCUGAGCAUCGUGUCAG | 873 | Exon 19 |
| TG-48-159 | GGCUCCAAGCCUAGAGGCUU | 874 | Exon 19 |
| TG-48-162 | GUUUUAAUUUCAGCACUGAC | 875 | Exon 20, Intron 19 |
| TG-48-169 | GGGGUCCGACUAUGCUGGUG | 876 | Exon 20 |
| TG-48-171 | GUCACCAGGCAUUGUGUCAG | 877 | Exon 21 |
| TG-48-178 | UCAGAAGAAGCCUCUGUGCU | 878 | Exon 21 |
| TG-48-182 | GUUGUCCUCUGAUGCCAGUG | 879 | Exon 22 |
| TG-48-195 | UACCUUGUUCAGAAACAGCC | 880 | Exon 23 |
| TG-48-196 | CUGAUGCCAGUGUGGUGUCA | 881 | Exon 24 |
| TG-48-197 | GUGGUUCUCUGAUGCCAGUG | 882 | Exon 24 |
| TG-48-199 | CUUGGGAGAAAACUCAAAGA | 883 | Intron 24 |
| TG-48-223 | UCCUGUGAUAGUGGUGGAGA | 884 | Exon 26 |
| TG-48-249 | GUAUGGUGUGGGGGUGAAGU | 885 | Intron 28 |
| TG-48-252 | GAAACCAGGACCCACUUGAG | 886 | Intron 28 |
| TG-48-254 | UAUUAUCUUCUGAAACCAGG | 887 | Intron 28 |
| TG-48-260 | GUAUGAGUUCAUUUACAUGA | 888 | Intron 28 |
| TG-48-265 | GUUGAAAGAAACCAAUGUAU | 889 | Intron 28 |
| TG-48-288 | UGGAUUCAUUGUGUAGCACC | 890 | Exon 33 |
| TG-48-291 | UUGUCUAGCGUGGGAUGCAU | 891 | Intron 33 |
| TG-48-293 | CAGCUGCAGAAGAGGACAGU | 892 | Intron 33 |
| TG-48-294 | UCUCAGCUGCAGAAGAGGAC | 893 | Intron 33 |
| TG-48-298 | CGUAAGAUUACUACUCAUGA | 894 | Intron 33 |
| TG-48-302 | UUUCCCACCUUGUGCCACAG | 895 | Exon 34, Intron 33 |
| TG-48-303 | UUCUAGGUUUGGAAAGCACU | 896 | Exon 35 |
| TG-48-306 | UCAAGCAGUGAGCAGCAGUC | 897 | Exon 35 |
| TG-48-317 | UGGAGCCCACACAAGCAGAU | 898 | Exon 36 |
| TG-48-337 | CAAAAUAUUUUGCUGAAAGU | 899 | Intron 37 |
| TG-48-346 | GACACCGAAGACAAUGAUCU | 900 | Intron 37 |
| TG-48-351 | CAAUAACAAGGAGCUGGGCU | 901 | Exon 38 |
| TG-48-361 | AAGUAGGUUGAUGCUUCACU | 902 | Exon 39, 3UTR |
| TG-48-373 | GUAUUAAGGUGUCAUAGCUA | 903 | Exon 39, 3UTR |

### Results

The 142 TTC spacers identified by the in silico filtering criteria (Table 15) were subjected to further experimental assessment in the examples that follow. FIG. 1 illustrates a schematic of the relative locations in the human LPA gene that these 142 spacers target. As depicted in FIG. 1, the majority of the 142 TTC spacers do not target the Kringle IV Type 2 (KIV-2) repeat region, demonstrating that the computational algorithm was able to exclude non-specific spacers with high off-target activity and identified unique spacers for targeting the human LPA locus. Notably, by applying the in silico filtering criteria as described above, these 142 spacers are unique targeting sequences that avoid any repetitive regions and would only induce a single edit within the human genome.

The identification of a highly specific spacer is believed to be particularly important for targeting the LPA locus for several reasons. First, the repetitive Kringle regions within the LPA coding sequence are believed to make the locus prone to chromosomal rearrangements. Specifically, it is possible that introducing a double stranded break within an LPA Kringle repeat would lead the DNA to undergo homology-dependent repair with another Kringle repeat, and thereby introduce a deleterious chromosomal rearrangement. Further, there is a high degree of similarity between LPA and the gene encoding plasminogen (PLG). The LPA and PLG genes are homologs that both contain Kringle repeat regions and share sequence homology in both the untranslated and coding regions (McLean, J., et al., Nature 1987; 330:132-137; Schmidt K., et al., J Lipid Res. 2016; 57 (8): 1339-1359). The human PLG and LPA genes are clustered together on chromosome 6, which also harbors the LPA-like pseudogene, LPAL2, which is yet another potential source of off-target editing. Therefore, the computational process described above is believed to be important for identifying CasX spacers capable of effectively editing the LPA locus in a highly specific manner, without excessive off-target effects.

### Example 2: Editing of Human LPA Locus in Human Liver Cell Line

Experiments were performed to test the ability of a CasX:gRNA system to edit the LPA locus in the HepG2 human liver cell line.

### Materials and Methods

### Lentiviral Plasmid Cloning, Lentivirus Production, and Lentivirus Transduction:

Lentiviral plasmid constructs with sequences coding for CasX protein 515, and guide scaffold variant 235 with LPA-targeting spacers were generated and cloned upstream of a P2A-mScarlet coding region on a lentiviral plasmid using standard molecular cloning techniques. The spacers utilized were a subset of the 142 spacers provided in Table 15 (but with thymine substituted for uracil in the sequences of Table 15), above, or a control spacer targeting the B2M locus. Cloned and sequence-validated constructs were midi-prepped and subjected to quality assessment prior to transfection into HEK293T cells for lentiviral production.

Lentiviral particles were produced by transfecting HEK293T at a confluency of 70%-90% using lipofectamine-based transfection of the CasX and gRNA plasmids, a lentiviral packaging plasmid, and VSV-G envelope plasmids. For particle production, media was changed 12 hours post-transfection, and virus was harvested 36-48 hours post-transfection. Viral supernatants were filtered using 0.45 μm membrane filters, diluted in media if appropriate, and added to HepG2 target cells cultured in HepG2 medium (EMEM with 10% FBS and 1% penicillin-streptomycin) at a multiplicity of infection (MOI) of about 1. Supplemental DEAE-Dextran was added at 8 μg/ml to enhance transduction efficiency, if necessary. Transduced cells were grown for three days in HepG2 medium in a 37° C. incubator with 5% CO₂. Cells were then harvested and genomic DNA was extracted.

### Assessment of Editing of IPA Gene by Next-Generation Sequencing (NGS):

Genomic DNA was amplified via PCR with primers specific to the target genomic location of interest to form a target amplicon. These primers contained additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 program. The percentage of reads modified in a window around the 3′ end of the spacer was determined. The editing activity of the CasX molecule was quantified as the total percent of reads that contained deletions anywhere within this window (Table 16). Each LPA spacer was assessed in 1-3 independent replicates. Table 16, below, provides the mean deletion rate, the standard deviation, and the number of replicates for the tested spacers.

### Results

The ability of CasX 515 and gRNA scaffold 235 to edit the LPA locus when delivered by lentivirus in HepG2 cells was assessed. Table 16, below, provides the level of editing (deletion rate) produced by constructs with each individual spacer, rounded to the nearest hundredth.

TABLE 16
Editing of LPA locus by CasX:gRNA

| | Deletion Rate | Standard | Number of |
|---|---|---|---|
| Spacer ID | (Mean) | Deviation | Replicates |
| B2M spacer (control) | 0.28 | 0.29 | 20 |
| TG-48-005 | 0.28 | 0 | 2 |
| TG-48-006 | 0.14 | 0.02 | 3 |
| TG-48-007 | 0.27 | 0.02 | 3 |
| TG-48-008 | 0.19 | 0.04 | 3 |
| TG-48-009 | 0.14 | 0.24 | 3 |
| TG-48-014 | 0.13 | 0.03 | 3 |
| TG-48-015 | 0.55 | 0.05 | 3 |
| TG-48-016 | 0.51 | 0.04 | 2 |
| TG-48-017 | 0.59 | 0.1 | 3 |
| TG-48-018 | 0.22 | 0.07 | 3 |
| TG-48-029 | 0.17 | 0.03 | 3 |
| TG-48-031 | 0.05 | 0 | 3 |
| TG-48-032 | 0.11 | 0 | 3 |
| TG-48-033 | 0.04 | 0.01 | 3 |
| TG-48-035 | 0 | 0 | 3 |
| TG-48-036 | 0.03 | 0.01 | 3 |
| TG-48-037 | 0.22 | 0.07 | 3 |
| TG-48-038 | 0.26 | 0.03 | 3 |
| TG-48-039 | 0.33 | 0.05 | 3 |
| TG-48-040 | 0.01 | 0 | 3 |
| TG-48-052 | 0.42 | 0.03 | 2 |
| TG-48-056 | 0.22 | 0.04 | 3 |
| TG-48-059 | 0.1 | 0.02 | 3 |
| TG-48-061 | 0.3 | 0.04 | 3 |
| TG-48-064 | 0.03 | 0 | 3 |
| TG-48-066 | 0.12 | 0.02 | 2 |
| TG-48-069 | 0.09 | 0.01 | 3 |
| TG-48-071 | 0.08 | 0.02 | 3 |
| TG-48-078 | 0 | 0 | 2 |
| TG-48-080 | 0.04 | 0.01 | 2 |
| TG-48-083 | 0.2 | 0.01 | 3 |
| TG-48-087 | 0.1 | 0.01 | 3 |
| TG-48-088 | 0.17 | 0.03 | 3 |
| TG-48-092 | 0.25 | 0.05 | 3 |
| TG-48-097 | 0.23 | 0.03 | 3 |
| TG-48-098 | 0.07 | 0.01 | 3 |
| TG-48-100 | 0.01 | 0 | 3 |
| TG-48-102 | 0.19 | 0.01 | 3 |
| TG-48-103 | 0.04 | 0.01 | 3 |
| TG-48-104 | 0.11 | 0.18 | 3 |
| TG-48-107 | 0.3 | 0.02 | 3 |
| TG-48-109 | 0.23 | 0.04 | 3 |
| TG-48-111 | 0.2 | 0.02 | 3 |
| TG-48-112 | 0.07 | 0.02 | 3 |
| TG-48-113 | 0.3 | 0.02 | 3 |
| TG-48-114 | 0.04 | 0.04 | 3 |
| TG-48-115 | 0.04 | 0.01 | 3 |
| TG-48-118 | 0 | 0 | 3 |
| TG-48-119 | 0 | 0 | 3 |
| TG-48-124 | 0.02 | 0.04 | 3 |
| TG-48-126 | 0.1 | 0.03 | 3 |
| TG-48-127 | 0.03 | 0.01 | 3 |
| TG-48-128 | 0.08 | 0.01 | 3 |
| TG-48-129 | 0.05 | 0.01 | 3 |
| TG-48-136 | 0.03 | 0.01 | 3 |
| TG-48-137 | 0 | 0 | 3 |
| TG-48-138 | 0.02 | 0 | 3 |
| TG-48-139 | 0.15 | 0.02 | 3 |
| TG-48-141 | 0.07 | 0.12 | 3 |
| TG-48-142 | 0.12 | 0.03 | 3 |
| TG-48-143 | 0.1 | 0.03 | 3 |
| TG-48-144 | 0.03 | 0.04 | 3 |
| TG-48-145 | 0.13 | 0.05 | 3 |
| TG-48-151 | 0.03 | 0.01 | 2 |
| TG-48-154 | 0.07 | 0.01 | 2 |
| TG-48-155 | 0.03 | 0.01 | 3 |
| TG-48-159 | 0.03 | 0 | 2 |
| TG-48-162 | 0 | 0 | 3 |
| TG-48-163 | 0.04 | 0 | 3 |
| TG-48-171 | 0.3 | 0.07 | 3 |
| TG-48-178 | 0.17 | 0.04 | 3 |
| TG-48-179 | 0.07 | 0.03 | 3 |
| TG-48-182 | 0.01 | 0.01 | 3 |
| TG-48-193 | 0.08 | 0.02 | 3 |
| TG-48-195 | 0.04 | 0.02 | 3 |
| TG-48-196 | 0.02 | 0.03 | 3 |
| TG-48-197 | 0.08 | 0.04 | 3 |
| TG-48-199 | 0.24 | 0.12 | 2 |
| TG-48-201 | 0.04 | NA | 1 |
| TG-48-213 | 0.05 | 0.03 | 3 |
| TG-48-222 | 0.44 | 0.08 | 3 |
| TG-48-223 | 0.07 | 0.08 | 3 |
| TG-48-229 | 0 | 0 | 3 |
| TG-48-235 | 0.05 | 0.09 | 3 |
| TG-48-236 | 0.04 | 0.01 | 3 |
| TG-48-239 | 0.22 | 0.08 | 3 |
| TG-48-249 | 0.2 | 0.01 | 3 |
| TG-48-250 | 0.18 | 0.02 | 3 |
| TG-48-252 | 0.04 | 0.01 | 3 |
| TG-48-254 | 0.01 | 0.01 | 2 |
| TG-48-255 | 0.08 | 0.03 | 3 |
| TG-48-256 | 0.07 | 0.01 | 3 |
| TG-48-260 | 0.06 | 0.08 | 3 |
| TG-48-261 | 0.01 | 0.01 | 3 |
| TG-48-262 | 0 | 0 | 3 |
| TG-48-264 | 0.02 | 0.03 | 3 |
| TG-48-265 | 0.18 | 0.01 | 3 |
| TG-48-267 | 0.14 | 0.03 | 3 |
| TG-48-280 | 0.48 | 0.24 | 3 |
| TG-48-281 | 0.36 | 0.05 | 2 |
| TG-48-283 | 0.14 | NA | 1 |
| TG-48-284 | 0.2 | 0.01 | 3 |
| TG-48-285 | 0.05 | 0.08 | 3 |
| TG-48-286 | 0.17 | 0.02 | 3 |
| TG-48-288 | 0.18 | 0.02 | 3 |
| TG-48-290 | 0.19 | 0.01 | 3 |
| TG-48-291 | 0.13 | 0 | 2 |
| TG-48-292 | 0.13 | 0.03 | 3 |
| TG-48-293 | 0.36 | 0.01 | 2 |
| TG-48-294 | 0.21 | 0.01 | 3 |
| TG-48-298 | 0.25 | 0.04 | 3 |
| TG-48-299 | 0.09 | 0.01 | 3 |
| TG-48-300 | 0.08 | 0.02 | 3 |
| TG-48-302 | 0.04 | 0.01 | 2 |
| TG-48-303 | 0.11 | 0.07 | 3 |
| TG-48-305 | 0.27 | 0.05 | 2 |
| TG-48-306 | 0.15 | 0.01 | 3 |
| TG-48-308 | 0.06 | 0.01 | 3 |
| TG-48-312 | 0.09 | 0.02 | 3 |
| TG-48-317 | 0.05 | 0.09 | 3 |
| TG-48-318 | 0.08 | 0.13 | 3 |
| TG-48-324 | 0.11 | 0.05 | 3 |
| TG-48-336 | 0.44 | 0.04 | 3 |
| TG-48-337 | 0 | 0 | 2 |
| TG-48-341 | 0.5 | 0.01 | 2 |
| TG-48-342 | 0.3 | 0.04 | 3 |
| TG-48-343 | 0.5 | 0.05 | 3 |
| TG-48-344 | 0.12 | 0.01 | 3 |
| TG-48-346 | 0.18 | 0 | 3 |
| TG-48-351 | 0.4 | 0.04 | 3 |
| TG-48-352 | 0.1 | 0.03 | 2 |
| TG-48-358 | 0.1 | 0.02 | 2 |
| TG-48-359 | 0.09 | 0.02 | 3 |
| TG-48-360 | 0.1 | 0.01 | 3 |
| TG-48-361 | 0.1 | 0.01 | 3 |
| TG-48-362 | 0.03 | 0 | 2 |
| TG-48-373 | 0.13 | 0.07 | 3 |
| TG-48-375 | 0.01 | 0 | 2 |

CasX 515 and gRNAs with many of the tested spacers successfully edited the LPA locus in HepG2 cells, with varying levels of activity. Nine of the tested spacers produced deletion rates greater than 40% (TG-48-017, TG-48-015, TG-48-016, TG-48-343, TG-48-341, TG-48-280, TG-48-336, TG-48-222, and TG-48-052). Spacer TG-48-017 produced the highest level of editing, with a deletion rate of approximately 59%.

Unexpectedly, three spacers complementary to LPA exon 2 (TG-48-124, TG-48-119, TG-48-118) showed very low levels of editing. Indeed, TG-48-119 and TG-48-118 showed the lowest levels of editing of the tested spacers. Surprisingly, spacers complementary to LPA exon 38 showed the high levels of editing.

The results demonstrate that CasX 515 was able to edit the LPA locus in HepG2 cells with gRNAs with a variety of spacer sequences.

### Example 3: Editing of Human LPA Locus in Primary Human Hepatocytes

Experiments were performed to test the ability of a CasX:gRNA system to edit the LPA locus in the primary human hepatocytes.

### Materials and Methods

Lentiviral plasmids were cloned and lentiviruses were produced as described in Example 2, above. The spacers used in the constructs were a subset of the spacers tested in Example 2 (with thymine substituted for uracil in the sequences of Table 15 that were utilized), chosen to represent a range of editing levels and positions in the LPA locus, or a control spacer targeting the B2M locus. Cells were transduced as described in Example 2, except that the primary human hepatocytes were transduced at a relatively high dose of lentivirus, with an MOI of approximately 6, and cells were grown for five days before they were harvested and genomic DNA was extracted.

Editing of the LPA gene was measured by NGS as described in Example 2. Each spacer was assessed once. Table 17, below, provides the deletion rate for each of the tested spacers.

### Results:

Table 17, below, provides the level of editing (deletion rate) produced by the constructs for each individual spacer, rounded to the nearest hundredth.

TABLE 17
Editing of LPA locus by CasX:gRNA

| Spacer ID | Deletion Rate |
|---|---|
| B2M spacer (control) | 0 |
| TG-48-222 | 0.24 |
| TG-48-351 | 0.56 |
| TG-48-280 | 0.72 |
| TG-48-080 | 0.17 |
| TG-48-171 | 0.26 |
| TG-48-305 | 0.38 |
| TG-48-239 | 0.23 |
| TG-48-284 | 0.47 |
| TG-48-178 | 0.31 |
| TG-48-118 | 0 |
| TG-48-119 | 0 |
| TG-48-124 | 0.05 |
| TG-48-139 | 0.06 |
| TG-48-145 | 0.14 |

CasX 515 and gRNAs with the tested spacers edited the LPA locus in primary human hepatocytes with varying levels of activity. Spacer TG-48-280 produced the highest level of editing, with a deletion rate of 72%, followed by spacer TG-48-351, with a deletion rate of 56%.

The spacers complementary to exon 2 (TG-48-124, TG-48-119, TG-48-118) produced low levels of editing, consistent with their performance in HepG2 cells as described in Example 2.

The results demonstrate that CasX 515 was able to edit the LPA locus in primary human hepatocytes with gRNAs with a variety of spacer sequences.

### Example 4: Functional Assessments of CasX: GRNA Systems Targeting the Human LPA Locus

Experiments were performed to test the ability of CasX:gRNA systems to edit the LPA locus, reduce levels of secreted apo (a), and reduce levels of LPA mRNA in cell-based assays. CasX protein 515 (SEQ ID NO: 5) and guide scaffold variant 235 (SEQ ID NO: 465) were used, along with the 142 LPA-targeting sequences provided in Table 15, above, with the encoded components delivered in a lentiviral-based system.

### Materials and Methods

LPA-targeting sequences were narrowed to 60 top hits with greatest activity by lentiviral screening in primary human hepatocytes (PHH) for secreted apo (a) knockdown, and in HepG2 cells for editing activity, using the methodology described in Example 2.

### Lentiviral Cloning and Transduction:

Lentiviral plasmids were cloned and lentiviruses were produced as described in Example 2, above. The LPA-targeting sequences used in the constructs were the 142 LPA-targeting sequences listed in Table 15 (but with thymine substituted for uracil in the sequences of Table 15), or a control targeting sequence targeting the B2M locus. Cells were transduced as described in Example 2, except that the primary human hepatocytes were transduced at a target MOI of at least 2, and cells were grown for six days before they were harvested. Briefly, 60,000 PHHs (Lonza lot #HUM183121) were plated onto collagen-coated 96-well plates and cultured in H+ media for the duration of the experiment. The day after plating, LV stocks were thawed and titers were normalized to a final volume of 50 uL. PHHs were then treated with LV and DEAE-Dextran (8 μg/mL) in order to maximize LV transduction efficiency. After 24 hours, a full media change was performed. The final media collection was performed 7 days after plating with the last full media change being performed 72 hours prior to collection. Five replicates were tested for each condition.

Assessment of apo (a) secretion by Western Blot using an automated capillary separation and detection system, Jess (Bio-Techne):

Briefly, primary human hepatocytes were transduced with lentivirus encoding for CasX 515 and an LPA-targeting gRNA. Six days after lentiviral transfection, and 72 hours after a media change, supernatant was harvested and the apo (a) secreted into the media was measured. Total protein was quantified and diluted to 2 μg/uL prior to Western blotting, and secreted apo (a) was detected using a monoclonal apo (a) antibody (Abcam: Ab208184). The amount of protein was determined using Compass software to calculate the area under the curve (AUC) for the apo (a) peak detected at ˜450 kDa. AUC values were compared to a standard curve of fixed apo (a) concentrations to quantify final protein concentrations. While a standard curve was not included for every Jess run, 2 or more calibrator samples were included across multiple Jess runs to 1) calculate protein concentrations based on a previous standard curve and 2) evaluate protein concentrations from multiple Jess runs.

### Results

The ability of CasX 515 and gRNA comprising scaffold 235 and an LPA-targeting sequence to reduce secretion of apo (a) was assessed. Table 18, below, provides the percent of apo (a) produced by primary human hepatocytes with each individual targeting sequence, rounded to the nearest hundredth, and mean deletion rate as determined in Example 2.

Table 19 provides a list of the 60 LPA-targeting sequences that were selected for further evaluation.

TABLE 18
Apo(a) levels relative to non-targeting
gRNA in primary human hepatocytes

| Targeting | | Deletion |
|---|---|---|
| sequence ID | % Apo(a) | Rate (Mean) |
| TG-48-193 | −69.60 | 0.07 |
| TG-48-223 | −64.04 | 0.07 |
| TG-48-239 | −63.83 | 0.22 |
| TG-48-284 | −52.99 | 0.20 |
| TG-48-171 | −50.80 | 0.25 |
| TG-48-169 | −50.60 | 0.38 |
| TG-48-351 | −48.88 | 0.40 |
| TG-48-151 | −46.89 | 0.03 |
| TG-48-358 | −45.83 | 0.10 |
| TG-48-360 | −45.73 | 0.10 |
| TG-48-229 | −44.73 | 0.00 |
| TG-48-087 | −44.62 | 0.10 |
| TG-48-141 | −43.92 | 0.21 |
| TG-48-178 | −42.74 | 0.13 |
| TG-48-107 | −40.99 | 0.30 |
| TG-48-299 | −40.83 | 0.09 |
| TG-48-088 | −40.06 | 0.17 |
| TG-48-127 | −39.30 | 0.04 |
| TG-48-142 | −39.04 | 0.12 |
| TG-48-336 | −38.54 | 0.44 |
| TG-48-009 | −37.70 | 0.42 |
| TG-48-056 | −37.37 | 0.22 |
| TG-48-245 | −35.97 | 0.19 |
| TG-48-317 | −31.65 | 0.15 |
| TG-48-235 | −31.23 | 0.15 |
| TG-48-179 | −30.14 | 0.04 |
| TG-48-196 | −28.93 | 0.06 |
| TG-48-305 | −28.67 | 0.27 |
| TG-48-300 | −27.91 | 0.08 |
| TG-48-288 | −27.16 | 0.18 |
| TG-48-145 | −27.14 | 0.13 |
| TG-48-104 | −26.98 | 0.31 |
| TG-48-083 | −22.55 | 0.20 |
| TG-48-109 | −22.26 | 0.23 |
| TG-48-303 | −21.77 | 0.11 |
| TG-48-324 | −21.61 | 0.11 |
| TG-48-102 | −21.37 | 0.19 |
| TG-48-197 | −19.46 | 0.21 |
| TG-48-236 | −19.40 | 0.04 |
| TG-48-375 | −18.17 | 0.01 |
| TG-48-318 | −17.96 | 0.23 |
| TG-48-071 | −17.82 | 0.08 |
| TG-48-222 | −17.68 | 0.44 |
| TG-48-124 | −17.38 | 0.02 |
| TG-48-312 | −17.28 | 0.09 |
| TG-48-285 | −17.07 | 0.14 |
| TG-48-035 | −17.01 | 0.00 |
| TG-48-280 | −16.73 | 0.34 |
| TG-48-361 | −16.21 | 0.10 |
| TG-48-199 | −15.25 | 0.11 |
| TG-48-252 | −15.15 | 0.03 |
| TG-48-283 | −14.79 | 0.13 |
| TG-48-114 | −14.37 | 0.04 |
| TG-48-112 | −12.97 | 0.07 |
| TG-48-352 | −11.91 | 0.11 |
| TG-48-155 | −11.54 | 0.03 |
| TG-48-136 | −11.27 | 0.03 |
| TG-48-111 | −10.58 | 0.20 |
| TG-48-015 | −10.06 | 0.55 |
| TG-48-286 | −9.36 | 0.17 |
| TG-48-302 | −9.31 | 0.03 |
| TG-48-017 | −8.68 | 0.50 |
| TG-48-159 | −7.55 | 0.03 |
| TG-48-119 | −7.40 | 0.00 |
| TG-48-359 | −5.40 | 0.09 |
| TG-48-143 | −5.25 | 0.10 |
| TG-48-342 | −4.84 | 0.30 |
| TG-48-144 | −4.05 | 0.03 |
| TG-48-341 | −4.00 | 0.49 |
| TG-48-126 | −3.29 | 0.10 |
| TG-48-033 | −2.70 | 0.04 |
| TG-48-346 | −2.13 | 0.18 |
| TG-48-291 | −1.90 | 0.13 |
| TG-48-298 | −1.40 | 0.25 |
| TG-48-007 | −0.30 | 0.27 |
| TG-48-213 | 0.48 | 0.04 |
| TG-48-005 | 0.52 | 0.28 |
| TG-48-306 | 2.37 | 0.15 |
| TG-48-016 | 2.89 | 0.51 |
| TG-48-254 | 3.11 | 0.06 |
| TG-48-267 | 3.44 | 0.14 |
| TG-48-201 | 3.51 | 0.04 |
| TG-48-100 | 3.54 | 0.01 |
| TG-48-290 | 3.92 | 0.19 |
| TG-48-080 | 5.04 | 0.04 |
| TG-48-097 | 7.94 | 0.23 |
| TG-48-182 | 9.22 | 0.04 |
| TG-48-195 | 10.96 | 0.02 |
| TG-48-250 | 11.49 | 0.14 |
| TG-48-139 | 12.28 | 0.15 |
| TG-48-162 | 12.38 | 0.00 |
| TG-48-103 | 13.38 | 0.04 |
| TG-48-113 | 13.58 | 0.30 |
| TG-48-292 | 14.49 | 0.13 |
| TG-48-118 | 14.87 | 0.00 |
| TG-48-129 | 15.33 | 0.05 |
| TG-48-308 | 16.64 | 0.06 |
| TG-48-343 | 18.22 | 0.50 |
| TG-48-294 | 18.83 | 0.21 |
| TG-48-255 | 19.53 | 0.06 |
| TG-48-008 | 21.55 | 0.19 |
| TG-48-154 | 21.70 | 0.07 |
| TG-48-337 | 24.99 | 0.00 |
| TG-48-163 | 25.84 | 0.04 |
| TG-48-115 | 26.45 | 0.04 |
| TG-48-293 | 27.17 | 0.35 |
| TG-48-059 | 27.55 | 0.10 |
| TG-48-032 | 28.34 | 0.11 |
| TG-48-260 | 31.49 | 0.06 |
| TG-48-368 | 33.06 | NA |
| TG-48-128 | 37.75 | 0.08 |
| TG-48-249 | 41.55 | 0.19 |
| TG-48-036 | 43.19 | 0.03 |
| TG-48-373 | 43.58 | 0.13 |
| TG-48-366 | 44.79 | NA |
| TG-48-078 | 46.22 | 0.00 |
| TG-48-256 | 46.40 | 0.07 |
| TG-48-092 | 47.31 | 0.25 |
| TG-48-098 | 52.34 | 0.07 |
| TG-48-037 | 52.56 | 0.22 |
| TG-48-039 | 52.91 | 0.33 |
| TG-48-362 | 57.62 | 0.04 |
| TG-48-344 | 59.46 | 0.12 |
| TG-48-006 | 59.83 | 0.14 |
| TG-48-264 | 63.13 | 0.02 |
| TG-48-014 | 64.08 | 0.13 |
| TG-48-052 | 64.10 | 0.43 |
| TG-48-262 | 64.24 | 0.00 |
| TG-48-261 | 64.54 | 0.01 |
| TG-48-038 | 66.49 | 0.26 |
| TG-48-138 | 75.17 | 0.02 |
| TG-48-029 | 75.22 | 0.17 |
| TG-48-281 | 77.11 | 0.37 |
| TG-48-031 | 79.35 | 0.05 |
| TG-48-137 | 85.10 | 0.01 |
| TG-48-061 | 89.25 | 0.31 |
| TG-48-040 | 92.16 | 0.01 |
| TG-48-066 | 98.11 | 0.12 |
| TG-48-064 | 121.58 | 0.03 |
| TG-48-018 | 188.77 | 0.23 |
| TG-48-265 | 198.86 | 0.19 |
| TG-48-069 | 207.22 | 0.09 |

TABLE 19
Top 60 LPA-targeting sequences

| Targeting sequence ID | SEQ ID NO. |
|---|---|
| TG-48-107 | 776 |
| TG-48-178 | 878 |
| TG-48-222 | 817 |
| TG-48-284 | 826 |
| TG-48-303 | 896 |
| TG-48-305 | 792 |
| TG-48-317 | 898 |
| TG-48-351 | 901 |
| TG-48-005 | 811 |
| TG-48-007 | 809 |
| TG-48-009 | 771 |
| TG-48-015 | 852 |
| TG-48-016 | 851 |
| TG-48-017 | 799 |
| TG-48-056 | 853 |
| TG-48-083 | 805 |
| TG-48-087 | 806 |
| TG-48-088 | 854 |
| TG-48-102 | 807 |
| TG-48-104 | 775 |
| TG-48-109 | 857 |
| TG-48-111 | 858 |
| TG-48-113 | 859 |
| TG-48-118 | 862 |
| TG-48-119 | 863 |
| TG-48-124 | 864 |
| TG-48-127 | 865 |
| TG-48-137 | 869 |
| TG-48-139 | 813 |
| TG-48-141 | 814 |
| TG-48-142 | 871 |
| TG-48-145 | 840 |
| TG-48-151 | 873 |
| TG-48-162 | 875 |
| TG-48-169 | 876 |
| TG-48-171 | 877 |
| TG-48-179 | 815 |
| TG-48-193 | 780 |
| TG-48-196 | 881 |
| TG-48-199 | 883 |
| TG-48-223 | 884 |
| TG-48-229 | 818 |
| TG-48-235 | 819 |
| TG-48-239 | 820 |
| TG-48-267 | 824 |
| TG-48-280 | 787 |
| TG-48-281 | 825 |
| TG-48-283 | 788 |
| TG-48-286 | 789 |
| TG-48-288 | 890 |
| TG-48-299 | 828 |
| TG-48-300 | 829 |
| TG-48-306 | 897 |
| TG-48-324 | 793 |
| TG-48-336 | 833 |
| TG-48-341 | 834 |
| TG-48-342 | 835 |
| TG-48-343 | 843 |
| TG-48-358 | 836 |
| TG-48-360 | 795 |

The results demonstrate that CasX 515 and an LPA-targeting gRNA were able to edit the LPA locus in primary human hepatocytes, thereby functionally reducing apo (a) secretion. The results of assays described herein were used to select LPA-targeting sequences that are highly effective for editing the LPA locus and achieving a marked decrease in secreted apo (a) levels.

### Example 5: Demonstration that Altering the UTR Sequences of the Engineered CasX mRNA can Affect CasX-Mediated Editing

5′ and 3′ UTRs are essential and required for efficient translation of mRNA. Here, experiments were performed to demonstrate that altering the 5′ and 3′ UTR sequences of the engineered CasX mRNA affects CasX-mediated editing at a target locus when CasX mRNA and targeting gRNAs were delivered in vitro via transfection.

### Materials and Methods

In Vitro Transcription (IVT) of CasX mRNA:

CasX 676 mRNA was generated by IVT. Briefly, constructs encoding for a 5′UTR region, a codon-optimized CasX 676 with flanking c-MYC NLSes, and a 3′UTR region were cloned into a plasmid containing a T7 promoter and 80-nucleotide poly (A) tail. The resulting plasmid was linearized prior to use for IVT reactions, which were carried out with CleanCap® AG and NI-methyl-pseudouridine. For the 5′ cap, the CleanCap® AG contains a m7G (5′) ppp (5′) mAG structure, where “m7G” denotes N7-methylguanosine, “mA” denotes 2′O-methyladenosine, and (5′) ppp (5′) denotes a 5′ to 5′ triphosphate bridge. An extra guanine nucleotide was incorporated following the CleanCap® AG to enhance transcription initiation, resulting in the incorporation of m7G (5) ppp (5′) mAGG as the full 5′ cap structure. Meanwhile, the substitution of the uridine ribonucleoside to N1-methyl-pseudouridine improves mRNA performance and reduces mRNA immunogenicity.

IVT reactions were subsequently subjected to DNase digestion to remove template DNA and purification using an oligo-dT column. In this example, two mRNAs encoding CasX 676 with different pairs of 5′ and 3′ UTRs were generated for assessment in vitro. The encoding sequences of the two CasX mRNA configurations are detailed in Table 20. Full-length RNA sequences encoding the CasX mRNA with the chemical modifications are listed in Table 21.

TABLE 20
Encoding sequences of the two CasX mRNA molecules assessed in this
example*

| CasX | Component | | | SEQ ID |
|---|---|---|---|---|
| mRNA ID | (ID) | Description | DNA sequence | NO |
| CasX 676 | 5′UTR | Human | ACTCTTCTGGTCCCCACAGACTCAGAGAGAA | 19733 |
| mRNA #1 | | hemoglobin | CCCGCCACC | |
| | | alpha (HBA) | | |
| | START | | ATGGCCCCTGCTGCCAAGAGAGTGAAGCTGG | 19734 |
| | codon + c- | | ATAGCAGA | |
| | MYC NLS | | | |
| | CasX 676 | | CAGGAGATCAAGCGGATTAATAAAATTCGGA | 19735 |
| | | | GAAGACTGGTGAAGGATTCTAACACAAAGAA | |
| | | | GGCTGGCAAGACACGGGGCCCTATGAAGACA | |
| | | | CTGCTGGTGAGAGTGATGACACCCGACCTGA | |
| | | | GAGAAAGACTGGAAAACCTGAGAAAGAAGCC | |
| | | | TGAGAATATCCCCCAGCCCATCAGCAACACA | |
| | | | AGCCGGGCCAACCTGAATAAGCTGCTGACCG | |
| | | | ACTACACCGAAATGAAGAAGGCCATCCTGCA | |
| | | | CGTGTATTGGGAAGAGTTCCAGAAAGACCCA | |
| | | | GTCGGCCTGATGAGCAGAGTGGCTCAGCCTG | |
| | | | CCAGCAAGAAGATCGATCAGAACAAGCTGAA | |
| | | | GCCCGAAATGGACGAGAAGGGGAACCTGACA | |
| | | | ACCGCCGGCTTTGCCTGTAGCCAGTGCGGCC | |
| | | | AGCCCCTGTTTGTGTACAAACTGGAACAGGT | |
| | | | GAGCGAAAAGGGCAAGGCTTACACGAATTAC | |
| | | | TTCGGCAGATGCAACGTGGCCGAGCACGAGA | |
| | | | AGCTGATCAAGCTGGCCCAGCTGAAGCCTGA | |
| | | | GAAGGATAGCGATGAGGCAGTGACATATTCC | |
| | | | CTGGGCAAGTTCGGACAGCGGGCCCTGGATT | |
| | | | TTTATTCCATTCATGTGACCAAGGAATCCAC | |
| | | | CCACCCCGTCAAGCCTCTTGCCCAAATTGCC | |
| | | | GGCAACAGATACGCCTCCAGCCCCGTGGGCA | |
| | | | AGGCCCTGAGCGACGCCTGTATGGGCACCAT | |
| | | | CGCCAGCTTCCTGTCTAAGTACCAGGACATT | |
| | | | ATCATCGAGCACCAGAAGGTGGTGAAGGGCA | |
| | | | ACCAGAAGAGACTGGAGAGCCTGCGCGAGCT | |
| | | | GGCCGGCAAGGAAAACCTGGAGTATCCTAGC | |
| | | | GTGACCCTGCCTCCTCAGCCTCATACAAAGG | |
| | | | AGGGCGTGGATGCCTACAACGAAGTGATCGC | |
| | | | CCGGGTGCGGATGTGGGTGAACCTGAATCTG | |
| | | | TGGCAGAAGCTGAAGCTGTCTAGAGACGACG | |
| | | | CCAAGCCCCTGCTGAGACTGAAGGGCTTCCC | |
| | | | CAGCTTCCCTCTGGTGGAGAGACAGGCAAAT | |
| | | | GAAGTGGACTGGTGGGACATGGTGTGTAACG | |
| | | | TGAAGAAGCTGATCAATGAGAAGAAGGAGGA | |
| | | | CGGCAAAGTGTTCTGGCAGAATCTGGCCGGC | |
| | | | TACAAGCGTCAGGAGGCCCTGCGGCCCTACC | |
| | | | TGAGCAGCGAGGAAGACAGAAAGAAGGGCAA | |
| | | | GAAGTTCGCCCGGTATCAGCTGGGGGACCTG | |
| | | | CTGCTGCACCTCGAGAAGAAGCACGGCGAAG | |
| | | | ACTGGGGGAAGGTGTACGATGAGGCCTGGGA | |
| | | | GCGGATCGATAAGAAGGTGGAGGGCCTGAGC | |
| | | | AAGCACATCAAGCTGGAGGAGGAACGGAGAT | |
| | | | CTGAGGACGCCCAGAGCAAGGCCGCCCTGAC | |
| | | | CGACTGGCTGAGAGCCAAGGCCAGCTTCGTC | |
| | | | ATCGAGGGGCTGAAGGAGGCCGACAAGGACG | |
| | | | AGTTCTGCCGGTGCGAACTGAAGCTGCAGAA | |
| | | | GTGGTACGGAGATCTGAGAGGCAAACCTTTC | |
| | | | GCCATCGAGGCCGAGAACAGCATCCTGGACA | |
| | | | TCAGCGGCTTCAGCAAGCAGTACAACTGCGC | |
| | | | CTTTATTTGGCAGAAGGACGGAGTGAAGAAG | |
| | | | CTGAACCTGTACCTGATCATCAACTATTTCA | |
| | | | AGGGCGGCAAGCTGAGATTCAAGAAGATCAA | |
| | | | GCCTGAAGCCTTCGAGGCCAACAGATTCTAC | |
| | | | ACCGTGATTAACAAGAAAAGCGGAGAGATCG | |
| | | | TGCCAATGGAAGTGAACTTCAACTTCGACGA | |
| | | | CCCTAACCTGATCATCCTGCCCCTGGCATTT | |
| | | | GGCAAGCGGCAGGGCAGAGAGTTCATCTGGA | |
| | | | ACGACCTGCTGTCTCTGGAGACCGGCAGCCT | |
| | | | GAAGCTGGCCAACGGCAGAGTGATCGAGAAG | |
| | | | ACACTGTACAACAGACGAACCAGACAAGACG | |
| | | | AGCCCGCCCTGTTTGTGGCCCTGACCTTCGA | |
| | | | GAGAAGAGAGGTGCTGGACAGCAGCAATATC | |
| | | | AAGCCTATGAACCTGATCGGCGTGGACCGGG | |
| | | | GCGAGAACATCCCTGCCGTGATCGCCCTTAC | |
| | | | CGACCCCGAGGGATGCCCTCTGAGCCGGTTT | |
| | | | AAAGACAGCCTGGGCAACCCTACCCACATCC | |
| | | | TGAGAATTGGCGAGTCCTACAAGGAGAAGCA | |
| | | | GAGAACCATCCAGGCCAAGAAGGAGGTGGAG | |
| | | | CAGCGGCGGGCTGGCGGCTACTCCCGGAAGT | |
| | | | ACGCCAGCAAGGCCAAGAACCTGGCCGACGA | |
| | | | CATGGTTAGAAATACCGCCAGAGACCTCCTG | |
| | | | TACTACGCTGTGACCCAGGACGCCATGCTGA | |
| | | | TCTTCGAGAACCTGAGCAGAGGCTTCGGCAG | |
| | | | ACAGGGCAAGAGAACCTTCATGGCCGAGAGA | |
| | | | CAGTACACCCGGATGGAGGACTGGCTGACCG | |
| | | | CCAAGCTGGCCTACGAGGGCCTGCCCTCTAA | |
| | | | GACCTACCTGTCCAAGACCTTGGCACAGTAC | |
| | | | ACCAGCAAGACATGCTCTAACTGCGGCTTCA | |
| | | | CAATCACGAGCGCCGACTACGACCGGGTGCT | |
| | | | GGAGAAACTGAAGAAGACCGCCACAGGCTGG | |
| | | | ATGACCACCATTAACGGCAAGGAGCTGAAGG | |
| | | | TGGAGGGCCAGATCACCTACTACAACAGGTA | |
| | | | CAAACGGCAGAACGTGGTGAAGGACCTGAGC | |
| | | | GTGGAACTGGATAGACTGAGCGAGGAAAGCG | |
| | | | TAAACAATGACATCAGCAGCTGGACCAAGGG | |
| | | | CCGGAGCGGCGAGGCCCTGAGCCTGCTGAAG | |
| | | | AAGAGATTCTCCCACAGACCAGTGCAGGAGA | |
| | | | AGTTCGTGTGTCTGAACTGCGGCTTCGAGAC | |
| | | | CCACGCCGACGAGCAAGCCGCCCTGAACATC | |
| | | | GCCCGGTCTTGGCTTTTCCTGCGGAGCCAGG | |
| | | | AGTACAAGAAGTACCAGACAAACAAGACCAC | |
| | | | AGGCAACACAGACAAGAGAGCCTTCGTCGAG | |
| | | | ACCTGGCAGAGCTTCTACAGAAAGAAGCTGA | |
| | | | AGGAGGTGTGGAAGCCTGCCGTG | |
| | c-MYC NLS + | | GGAAGCCCCGCTGCCAAGAGAGTGAAGCTGG | 19736 |
| | STOP | | ACTAATAGATAA | |
| | codons | | | |
| | 3′UTR | Human HBA | GCTGGAGCCTCGGTGGCCATGCTTCTTGCCC | 19737 |
| | | | CTTGGGCCTCCCCCCAGCCCCTCCTCCCCTT | |
| | | | CCTGCACCCGTACCCCCGTGGTCTTTGAATA | |
| | | | AAGTCTGAGTGGGCGGCA | |
| | Poly(A) tail | | AAAAAAAAAAAAAAAAAAAAAAAAAAAAAAA | 19738 |
| | | | AAAAAAAAAAAAAAAAAAAAAAAAAAAAAAA | |
| | | | AAAAAAAAAAAAAAAAAA | |
| CasX 676 | 5′UTR + | Synthetic | AAATAAGAGAGAAAAGAAGAGTAAGAAGAAA | 19739 |
| mRNA #2 | Kozak | | TATAAGAGCCACC | |
| | sequence | | | |
| | (underlined) | | | |
| | START | | ATGGCCCCTGCTGCCAAGAGAGTGAAGCTGG | 19734 |
| | codon + c- | | ATAGCAGA | |
| | MYC NLS | | | |
| | CasX 676 | | CAGGAGATCAAGCGGATTAATAAAATTCGGA | 19735 |
| | | | GAAGACTGGTGAAGGATTCTAACACAAAGAA | |
| | | | GGCTGGCAAGACACGGGGCCCTATGAAGACA | |
| | | | CTGCTGGTGAGAGTGATGACACCCGACCTGA | |
| | | | GAGAAAGACTGGAAAACCTGAGAAAGAAGCC | |
| | | | TGAGAATATCCCCCAGCCCATCAGCAACACA | |
| | | | AGCCGGGCCAACCTGAATAAGCTGCTGACCG | |
| | | | ACTACACCGAAATGAAGAAGGCCATCCTGCA | |
| | | | CGTGTATTGGGAAGAGTTCCAGAAAGACCCA | |
| | | | GTCGGCCTGATGAGCAGAGTGGCTCAGCCTG | |
| | | | CCAGCAAGAAGATCGATCAGAACAAGCTGAA | |
| | | | GCCCGAAATGGACGAGAAGGGGAACCTGACA | |
| | | | ACCGCCGGCTTTGCCTGTAGCCAGTGCGGCC | |
| | | | AGCCCCTGTTTGTGTACAAACTGGAACAGGT | |
| | | | GAGCGAAAAGGGCAAGGCTTACACGAATTAC | |
| | | | TTCGGCAGATGCAACGTGGCCGAGCACGAGA | |
| | | | AGCTGATCAAGCTGGCCCAGCTGAAGCCTGA | |
| | | | GAAGGATAGCGATGAGGCAGTGACATATTCC | |
| | | | CTGGGCAAGTTCGGACAGCGGGCCCTGGATT | |
| | | | TTTATTCCATTCATGTGACCAAGGAATCCAC | |
| | | | CCACCCCGTCAAGCCTCTTGCCCAAATTGCC | |
| | | | GGCAACAGATACGCCTCCAGCCCCGTGGGCA | |
| | | | AGGCCCTGAGCGACGCCTGTATGGGCACCAT | |
| | | | CGCCAGCTTCCTGTCTAAGTACCAGGACATT | |
| | | | ATCATCGAGCACCAGAAGGTGGTGAAGGGCA | |
| | | | ACCAGAAGAGACTGGAGAGCCTGCGCGAGCT | |
| | | | GGCCGGCAAGGAAAACCTGGAGTATCCTAGC | |
| | | | GTGACCCTGCCTCCTCAGCCTCATACAAAGG | |
| | | | AGGGCGTGGATGCCTACAACGAAGTGATCGC | |
| | | | CCGGGTGCGGATGTGGGTGAACCTGAATCTG | |
| | | | TGGCAGAAGCTGAAGCTGTCTAGAGACGACG | |
| | | | CCAAGCCCCTGCTGAGACTGAAGGGCTTCCC | |
| | | | CAGCTTCCCTCTGGTGGAGAGACAGGCAAAT | |
| | | | GAAGTGGACTGGTGGGACATGGTGTGTAACG | |
| | | | TGAAGAAGCTGATCAATGAGAAGAAGGAGGA | |
| | | | CGGCAAAGTGTTCTGGCAGAATCTGGCCGGC | |
| | | | TACAAGCGTCAGGAGGCCCTGCGGCCCTACC | |
| | | | TGAGCAGCGAGGAAGACAGAAAGAAGGGCAA | |
| | | | GAAGTTCGCCCGGTATCAGCTGGGGGACCTG | |
| | | | CTGCTGCACCTCGAGAAGAAGCACGGCGAAG | |
| | | | ACTGGGGGAAGGTGTACGATGAGGCCTGGGA | |
| | | | GCGGATCGATAAGAAGGTGGAGGGCCTGAGC | |
| | | | AAGCACATCAAGCTGGAGGAGGAACGGAGAT | |
| | | | CTGAGGACGCCCAGAGCAAGGCCGCCCTGAC | |
| | | | CGACTGGCTGAGAGCCAAGGCCAGCTTCGTC | |
| | | | ATCGAGGGGCTGAAGGAGGCCGACAAGGACG | |
| | | | AGTTCTGCCGGTGCGAACTGAAGCTGCAGAA | |
| | | | GTGGTACGGAGATCTGAGAGGCAAACCTTTC | |
| | | | GCCATCGAGGCCGAGAACAGCATCCTGGACA | |
| | | | TCAGCGGCTTCAGCAAGCAGTACAACTGCGC | |
| | | | CTTTATTTGGCAGAAGGACGGAGTGAAGAAG | |
| | | | CTGAACCTGTACCTGATCATCAACTATTTCA | |
| | | | AGGGCGGCAAGCTGAGATTCAAGAAGATCAA | |
| | | | GCCTGAAGCCTTCGAGGCCAACAGATTCTAC | |
| | | | ACCGTGATTAACAAGAAAAGCGGAGAGATCG | |
| | | | TGCCAATGGAAGTGAACTTCAACTTCGACGA | |
| | | | CCCTAACCTGATCATCCTGCCCCTGGCATTT | |
| | | | GGCAAGCGGCAGGGCAGAGAGTTCATCTGGA | |
| | | | ACGACCTGCTGTCTCTGGAGACCGGCAGCCT | |
| | | | GAAGCTGGCCAACGGCAGAGTGATCGAGAAG | |
| | | | ACACTGTACAACAGACGAACCAGACAAGACG | |
| | | | AGCCCGCCCTGTTTGTGGCCCTGACCTTCGA | |
| | | | GAGAAGAGAGGTGCTGGACAGCAGCAATATC | |
| | | | AAGCCTATGAACCTGATCGGCGTGGACCGGG | |
| | | | GCGAGAACATCCCTGCCGTGATCGCCCTTAC | |
| | | | CGACCCCGAGGGATGCCCTCTGAGCCGGTTT | |
| | | | AAAGACAGCCTGGGCAACCCTACCCACATCC | |
| | | | TGAGAATTGGCGAGTCCTACAAGGAGAAGCA | |
| | | | GAGAACCATCCAGGCCAAGAAGGAGGTGGAG | |
| | | | CAGCGGCGGGCTGGCGGCTACTCCCGGAAGT | |
| | | | ACGCCAGCAAGGCCAAGAACCTGGCCGACGA | |
| | | | CATGGTTAGAAATACCGCCAGAGACCTCCTG | |
| | | | TACTACGCTGTGACCCAGGACGCCATGCTGA | |
| | | | TCTTCGAGAACCTGAGCAGAGGCTTCGGCAG | |
| | | | ACAGGGCAAGAGAACCTTCATGGCCGAGAGA | |
| | | | CAGTACACCCGGATGGAGGACTGGCTGACCG | |
| | | | CCAAGCTGGCCTACGAGGGCCTGCCCTCTAA | |
| | | | GACCTACCTGTCCAAGACCTTGGCACAGTAC | |
| | | | ACCAGCAAGACATGCTCTAACTGCGGCTTCA | |
| | | | CAATCACGAGCGCCGACTACGACCGGGTGCT | |
| | | | GGAGAAACTGAAGAAGACCGCCACAGGCTGG | |
| | | | ATGACCACCATTAACGGCAAGGAGCTGAAGG | |
| | | | TGGAGGGCCAGATCACCTACTACAACAGGTA | |
| | | | CAAACGGCAGAACGTGGTGAAGGACCTGAGC | |
| | | | GTGGAACTGGATAGACTGAGCGAGGAAAGCG | |
| | | | TAAACAATGACATCAGCAGCTGGACCAAGGG | |
| | | | CCGGAGCGGCGAGGCCCTGAGCCTGCTGAAG | |
| | | | AAGAGATTCTCCCACAGACCAGTGCAGGAGA | |
| | | | AGTTCGTGTGTCTGAACTGCGGCTTCGAGAC | |
| | | | CCACGCCGACGAGCAAGCCGCCCTGAACATC | |
| | | | GCCCGGTCTTGGCTTTTCCTGCGGAGCCAGG | |
| | | | AGTACAAGAAGTACCAGACAAACAAGACCAC | |
| | | | AGGCAACACAGACAAGAGAGCCTTCGTCGAG | |
| | | | ACCTGGCAGAGCTTCTACAGAAAGAAGCTGA | |
| | | | AGGAGGTGTGGAAGCCTGCCGTG | |
| | c-MYC NLS + | | GGAAGCCCCGCTGCCAAGAGAGTGAAGCTGG | 19736 |
| | STOP | | ACTAATAGATAA | |
| | codons | | | |
| | 3′UTR | Mouse HBA | GCTGCCTTCTGCGGGGCTTGCCTTCTGGCCA | 19740 |
| | | | TGCCCTTCTTCTCTCCCTTGCACCTGTACCT | |
| | | | CTTGGTCTTTGAATAAAGCCTGAGTAGGAAG | |
| | XbaI | | TCTAG | 19741 |
| | restriction | | | |
| | site (partial) | | | |
| | Poly(A) tail | | AAAAAAAAAAAAAAAAAAAAAAAAAAAAAAA | 19738 |
| | | | AAAAAAAAAAAAAAAAAAAAAAAAAAAAAAA | |
| | | | AAAAAAAAAAAAAAAAAA | |

*Components are listed in a 5′ to 3′ order within the constructs

TABLE 21
Full-length RNA sequences of CasX mRNA molecules assessed in this
example. The 5′ cap (m7G(5′)ppp(5′)mAG), discussed in the example herein, is not
shown in the table. Modification ‘mψ’ = N1-methyl-pseudouridine

| CasX | | SEQ ID |
|---|---|---|
| mRNA | RNA sequence | NO |
| CasX 676 | ACmψCmψmψCmψGGmψCCCCACAGACmψCAGAGAGAACCCGCCACCAmψGGCCCC | 19742 |
| mRNA #1 | mψGCmψGCCAAGAGAGmψGAAGCmψGGAmψAGCAGACAGGAGAmψCAAGCGGAmψ | |
| | mψAAmψAAAAmψmψCGGAGAAGACmψGGmψGAAGGAmψmψCmψAACACAAAGAAG | |
| | GCmψGGCAAGACACGGGGCCCmψAmψGAAGACACmψGCmψGGmψGAGAGmψGAmψ | |
| | GACACCCGACCmψGAGAGAAAGACmψGGAAAACCmψGAGAAAGAAGCCmψGAGAA | |
| | mψAmψCCCCCAGCCCAmψCAGCAACACAAGCCGGGCCAACCmψGAAmψAAGCmψG | |
| | CmψGACCGACmψACACCGAAAmψGAAGAAGGCCAmψCCmψGCACGmψGmψAmψmψ | |
| | GGGAAGAGmψmψCCAGAAAGACCCAGmψCGGCCmψGAmψGAGCAGAGmψGGCmψC | |
| | AGCCmψGCCAGCAAGAAGAmψCGAmψCAGAACAAGCmψGAAGCCCGAAAmψGGAC | |
| | GAGAAGGGGAACCmψGACAACCGCCGGCmψmψmψGCCmψGmψAGCCAGmψGCGGC | |
| | CAGCCCCmψGmψmψmψGmψGmψACAAACmψGGAACAGGmψGAGCGAAAAGGGCAA | |
| | GGCmψmψACACGAAmψmψACmψmψCGGCAGAmψGCAACGmψGGCCGAGCACGAGA | |
| | AGCmψGAmψCAAGCmψGGCCCAGCmψGAAGCCmψGAGAAGGAmψAGCGAmψGAGG | |
| | CAGmψGACAmψAmψmψCCCmψGGGCAAGmψmψCGGACAGCGGGCCCmψGGAmψmψ | |
| | mψmψmψAmψmψCCAmψmψCAmψGmψGACCAAGGAAmψCCACCCACCCCGmψCAAG | |
| | CCmψCmψmψGCCCAAAmψmψGCCGGCAACAGAmψACGCCmψCCAGCCCCGmψGGG | |
| | CAAGGCCCmψGAGCGACGCCmψGmψAmψGGGCACCAmψCGCCAGCmψmψCCmψGm | |
| | ψCmψAAGmψACCAGGACAmψmψAmψCAmψCGAGCACCAGAAGGmψGGmψGAAGGG | |
| | CAACCAGAAGAGACmψGGAGAGCCmψGCGCGAGCmψGGCCGGCAAGGAAAACCmψ | |
| | GGAGmψAmψCCmψAGCGmψGACCCmψGCCmψCCmψCAGCCmψCAmψACAAAGGAG | |
| | GGCGmψGGAmψGCCmψACAACGAAGmψGAmψCGCCCGGGmψGCGGAmψGmψGGGm | |
| | ψGAACCmψGAAmψCmψGmψGGCAGAAGCmψGAAGCmψGmψCmψAGAGACGACGCC | |
| | AAGCCCCmψGCmψGAGACmψGAAGGGCmψmψCCCCAGCmψmψCCCmψCmψGGmψG | |
| | GAGAGACAGGCAAAmψGAAGmψGGACmψGGmψGGGACAmψGGmψGmψGmψAACGm | |
| | ψGAAGAAGCmψGAmψCAAmψGAGAAGAAGGAGGACGGCAAAGmψGmψmψCmψGGC | |
| | AGAAmψCmψGGCCGGCmψACAAGCGmψCAGGAGGCCCmψGCGGCCCmψACCmψGA | |
| | GCAGCGAGGAAGACAGAAAGAAGGGCAAGAAGmψmψCGCCCGGmψAmψCAGCmψG | |
| | GGGGACCmψGCmψGCmψGCACCmψCGAGAAGAAGCACGGCGAAGACmψGGGGGAA | |
| | GGmψGmψACGAmψGAGGCCmψGGGAGCGGAmψCGAmψAAGAAGGmψGGAGGGCCm | |
| | ψGAGCAAGCACAmψCAAGCmψGGAGGAGGAACGGAGAmψCmψGAGGACGCCCAGA | |
| | GCAAGGCCGCCCmψGACCGACmψGGCmψGAGAGCCAAGGCCAGCmψmψCGmψCAm | |
| | ψCGAGGGGCmψGAAGGAGGCCGACAAGGACGAGmψmψCmψGCCGGmψGCGAACmψ | |
| | GAAGCmψGCAGAAGmψGGmψACGGAGAmψCmψGAGAGGCAAACCmψmψmψCGCCA | |
| | mψCGAGGCCGAGAACAGCAmψCCmψGGACAmψCAGCGGCmψmψCAGCAAGCAGmψ | |
| | ACAACmψGCGCCmψmψmψAmψmψmψGGCAGAAGGACGGAGmψGAAGAAGCmψGAA | |
| | CCmψGmψACCmψGAmψCAmψCAACmψAmψmψmψCAAGGGCGGCAAGCmψGAGAmψ | |
| | mψCAAGAAGAmψCAAGCCmψGAAGCCmψmψCGAGGCCAACAGAmψmψCmψACACC | |
| | GmψGAmψmψAACAAGAAAAGCGGAGAGAmψCGmψGCCAAmψGGAAGmψGAACmψm | |
| | ψCAACmψmψCGACGACCCmψAACCmψGAmψCAmψCCmψGCCCCmψGGCAmψmψmψ | |
| | GGCAAGCGGCAGGGCAGAGAGmψmψCAmψCmψGGAACGACCmψGCmψGmψCmψCm | |
| | ψGGAGACCGGCAGCCmψGAAGCmψGGCCAACGGCAGAGmψGAmψCGAGAAGACAC | |
| | mψGmψACAACAGACGAACCAGACAAGACGAGCCCGCCCmψGmψmψmψGmψGGCCC | |
| | mψGACCmψmψCGAGAGAAGAGAGGmψGCmψGGACAGCAGCAAmψAmψCAAGCCmψ | |
| | AmψGAACCmψGAmψCGGCGmψGGACCGGGGCGAGAACAmψCCCmψGCCGmψGAmψ | |
| | CGCCCmψmψACCGACCCCGAGGGAmψGCCCmψCmψGAGCCGGmψmψmψAAAGACA | |
| | GCCmψGGGCAACCCmψACCCACAmψCCmψGAGAAmψmψGGCGAGmψCCmψACAAG | |
| | GAGAAGCAGAGAACCAmψCCAGGCCAAGAAGGAGGmψGGAGCAGCGGCGGGCmψG | |
| | GCGGCmψACmψCCCGGAAGmψACGCCAGCAAGGCCAAGAACCmψGGCCGACGACA | |
| | mψGGmψmψAGAAAmψACCGCCAGAGACCmψCCmψGmψACmψACGCmψGmψGACCC | |
| | AGGACGCCAmψGCmψGAmψCmψmψCGAGAACCmyGAGCAGAGGCmψmψCGGCAGA | |
| | CAGGGCAAGAGAACCmψmψCAmψGGCCGAGAGACAGmψACACCCGGAmψGGAGGA | |
| | CmψGGCmψGACCGCCAAGCmψGGCCmψACGAGGGCCmψGCCCmψCmψAAGACCmψ | |
| | ACCmψGmψCCAAGACCmψmψGGCACAGmψACACCAGCAAGACAmψGCmψCmψAAC | |
| | mψGCGGCmψmψCACAAmψCACGAGCGCCGACmψACGACCGGGmψGCmψGGAGAAA | |
| | CmψGAAGAAGACCGCCACAGGCmψGGAmψGACCACCAmψmψAACGGCAAGGAGCm | |
| | ψGAAGGmψGGAGGGCCAGAmψCACCmψACmψACAACAGGmψACAAACGGCAGAAC | |
| | GmψGGmψGAAGGACCmψGAGCGmψGGAACmψGGAmψAGACmψGAGCGAGGAAAGC | |
| | GmψAAACAAmψGACAmψCAGCAGCmψGGACCAAGGGCCGGAGCGGCGAGGCCCmψ | |
| | GAGCCmψGCmψGAAGAAGAGAmψmψCmψCCCACAGACCAGmψGCAGGAGAAGmψm | |
| | ψCGmψGmψGmψCmψGAACmψGCGGCmψmψCGAGACCCACGCCGACGAGCAAGCCG | |
| | CCCmψGAACAmψCGCCCGGmψCmψmψGGCmψmψmψmψCCmψGCGGAGCCAGGAGm | |
| | ψACAAGAAGmψACCAGACAAACAAGACCACAGGCAACACAGACAAGAGAGCCmψm | |
| | ψCGmψCGAGACCmψGGCAGAGCmψmψCmψACAGAAAGAAGCmψGAAGGAGGmψGm | |
| | ψGGAAGCCmψGCCGmψGGGAAGCCCCGCmψGCCAAGAGAGmψGAAGCmψGGACmψ | |
| | AAmψAGAmψAAGCmψGGAGCCmψCGGmψGGCCAmψGCmψmψCmψmψGCCCCmψmψ | |
| | GGGCCmψCCCCCCAGCCCCmψCCmψCCCCmψmψCCmψGCACCCGmψACCCCCGmψ | |
| | GGmψCmψmψmψGAAmψAAAGmψCmψGAGmψGGGCGGCAAAAAAAAAAAAAAAAAA | |
| | AAAAAAAAAAAAAAAAAAAAAAAAAAAAAAAAAAAAAAAAAAAAAAAAAAAAAAA | |
| | AAAAAAA | |
| CasX 676 | AAAmψAAGAGAGAAAAGAAGAGmψAAGAAGAAAmψAmψAAGAGCCACCAmψGGCC | 19743 |
| mRNA #2 | CCmψGCmψGCCAAGAGAGmψGAAGCmψGGAmψAGCAGACAGGAGAmψCAAGCGGA | |
| | mψmψAAmψAAAAmψmψCGGAGAAGACmψGGmψGAAGGAmψmψCmψAACACAAAGA | |
| | AGGCmψGGCAAGACACGGGGCCCmψAmψGAAGACACmψGCmψGGmψGAGAGmψGA | |
| | mψGACACCCGACCmψGAGAGAAAGACmψGGAAAACCmψGAGAAAGAAGCCmψGAG | |
| | AAmψAmψCCCCCAGCCCAmψCAGCAACACAAGCCGGGCCAACCmψGAAmψAAGCm | |
| | ψGCmψGACCGACmψACACCGAAAmψGAAGAAGGCCAmψCCmψGCACGmψGmψAmψ | |
| | mψGGGAAGAGmψmψCCAGAAAGACCCAGmψCGGCCmψGAmψGAGCAGAGmψGGCm | |
| | ψCAGCCmψGCCAGCAAGAAGAmψCGAmψCAGAACAAGCmψGAAGCCCGAAAmψGG | |
| | ACGAGAAGGGGAACCmψGACAACCGCCGGCmψmψmψGCCmψGmψAGCCAGmψGCG | |
| | GCCAGCCCCmψGmψmψmψGmψGmψACAAACmψGGAACAGGmψGAGCGAAAAGGGC | |
| | AAGGCmψmψACACGAAmψmψACmψmψCGGCAGAmψGCAACGmψGGCCGAGCACGA | |
| | GAAGCmψGAmψCAAGCmψGGCCCAGCmψGAAGCCmψGAGAAGGAmψAGCGAmψGA | |
| | GGCAGmψGACAmψAmψmψCCCmψGGGCAAGmψmψCGGACAGCGGGCCCmψGGAmψ | |
| | mψmψmψmψAmψmψCCAmψmψCAmψGmψGACCAAGGAAmψCCACCCACCCCGmψCA | |
| | AGCCmψCmψmψGCCCAAAmψmψGCCGGCAACAGAmψACGCCmψCCAGCCCCGmψG | |
| | GGCAAGGCCCmψGAGCGACGCCmψGmψAmψGGGCACCAmψCGCCAGCmψmψCCmψ | |
| | GmψCmψAAGmψACCAGGACAmψmψAmψCAmψCGAGCACCAGAAGGmψGGmψGAAG | |
| | GGCAACCAGAAGAGACmψGGAGAGCCmψGCGCGAGCmψGGCCGGCAAGGAAAACC | |
| | mψGGAGmψAmψCCmψAGCGmψGACCCmψGCCmψCCmψCAGCCmψCAmψACAAAGG | |
| | AGGGCGmψGGAmwGCCmψACAACGAAGmψGAmψCGCCCGGGmψGCGGAmψGmψGG | |
| | GmψGAACCmψGAAmψCmψGmψGGCAGAAGCmψGAAGCmψGmψCmψAGAGACGACG | |
| | CCAAGCCCCmψGCmψGAGACmψGAAGGGCmψmψCCCCAGCmψmψCCCmψCmψGGm | |
| | ψGGAGAGACAGGCAAAmψGAAGmyGGACmψGGmψGGGACAmψGGmψGmψGmψAAC | |
| | GmψGAAGAAGCmψGAmψCAAmψGAGAAGAAGGAGGACGGCAAAGmψGmψmψCmψG | |
| | GCAGAAmψCmψGGCCGGCmψACAAGCGmψCAGGAGGCCCmψGCGGCCCmψACCmψ | |
| | GAGCAGCGAGGAAGACAGAAAGAAGGGCAAGAAGmψmψCGCCCGGmψAmψCAGCm | |
| | ψGGGGGACCmψGCmψGCmψGCACCmψCGAGAAGAAGCACGGCGAAGACmψGGGGG | |
| | AAGGmψGmψACGAmψGAGGCCmψGGGAGCGGAmψCGAmψAAGAAGGmψGGAGGGC | |
| | CmψGAGCAAGCACAmψCAAGCmψGGAGGAGGAACGGAGAmψCmψGAGGACGCCCA | |
| | GAGCAAGGCCGCCCmψGACCGACmψGGCmψGAGAGCCAAGGCCAGCmψmψCGmψC | |
| | AmψCGAGGGGCmψGAAGGAGGCCGACAAGGACGAGmψmψCmψGCCGGmψGCGAAC | |
| | mψGAAGCmψGCAGAAGmψGGmψACGGAGAmψCmψGAGAGGCAAACCmψmψmψCGC | |
| | CAmψCGAGGCCGAGAACAGCAmψCCmψGGACAmψCAGCGGCmψmψCAGCAAGCAG | |
| | mψACAACmψGCGCCmψmψmψAmψmψmψGGCAGAAGGACGGAGmψGAAGAAGCmψG | |
| | AACCmψGmψACCmψGAmψCAmψCAACmψAmψmψmψCAAGGGCGGCAAGCmψGAGA | |
| | mψmψCAAGAAGAmψCAAGCCmψGAAGCCmψmψCGAGGCCAACAGAmψmψCmψACA | |
| | CCGmψGAmψmψAACAAGAAAAGCGGAGAGAmψCGmψGCCAAmψGGAAGmψGAACm | |
| | ψmψCAACmψmψCGACGACCCmψAACCmψGAmψCAmψCCmψGCCCCmψGGCAmψmψ | |
| | mψGGCAAGCGGCAGGGCAGAGAGmψmψCAmψCmψGGAACGACCmψGCmψGmψCmψ | |
| | CmψGGAGACCGGCAGCCmψGAAGCmψGGCCAACGGCAGAGmψGAmψCGAGAAGAC | |
| | ACmψGmψACAACAGACGAACCAGACAAGACGAGCCCGCCCmψGmψmψmψGmψGGC | |
| | CCmψGACCmψmψCGAGAGAAGAGAGGmψGCmψGGACAGCAGCAAmψAmψCAAGCC | |
| | mψAmψGAACCmψGAmψCGGCGmψGGACCGGGGCGAGAACAmψCCCmψGCCGmψGA | |
| | mψCGCCCmψmψACCGACCCCGAGGGAmψGCCCmψCmψGAGCCGGmψmψmψAAAGA | |
| | CAGCCmψGGGCAACCCmψACCCACAmψCCmψGAGAAmψmψGGCGAGmψCCmψACA | |
| | AGGAGAAGCAGAGAACCAmψCCAGGCCAAGAAGGAGGmψGGAGCAGCGGCGGGCm | |
| | ψGGCGGCmψACmψCCCGGAAGmψACGCCAGCAAGGCCAAGAACCmψGGCCGACGA | |
| | CAmψGGmψmψAGAAAmψACCGCCAGAGACCmψCCmψGmψACmψACGCmψGmψGAC | |
| | CCAGGACGCCAmψGCmψGAmψCmψmψCGAGAACCmψGAGCAGAGGCmψmψCGGCA | |
| | GACAGGGCAAGAGAACCmψmψCAmψGGCCGAGAGACAGmψACACCCGGAmψGGAG | |
| | GACmψGGCmψGACCGCCAAGCmψGGCCmψACGAGGGCCmψGCCCmψCmψAAGACC | |
| | mψACCmψGmψCCAAGACCmψmψGGCACAGmψACACCAGCAAGACAmψGCmψCmψA | |
| | ACmψGCGGCmψmψCACAAmψCACGAGCGCCGACmψACGACCGGGmψGCmψGGAGA | |
| | AACmψGAAGAAGACCGCCACAGGCmψGGAmψGACCACCAmψmψAACGGCAAGGAG | |
| | CmψGAAGGmψGGAGGGCCAGAmψCACCmψACmψACAACAGGmψACAAACGGCAGA | |
| | ACGmψGGmwGAAGGACCmψGAGCGmψGGAACmψGGAmψAGACmψGAGCGAGGAAA | |
| | GCGmψAAACAAmψGACAmψCAGCAGCmψGGACCAAGGGCCGGAGCGGCGAGGCCC | |
| | mψGAGCCmψGCmψGAAGAAGAGAmψmψCmψCCCACAGACCAGmψGCAGGAGAAGm | |
| | ψmψCGmψGmψGmψCmψGAACmψGCGGCmψmψCGAGACCCACGCCGACGAGCAAGC | |
| | CGCCCmψGAACAmψCGCCCGGmψCmψmψGGCmψmψmψmψCCmψGCGGAGCCAGGA | |
| | GmψACAAGAAGmψACCAGACAAACAAGACCACAGGCAACACAGACAAGAGAGCCm | |
| | ψmψCGmψCGAGACCmψGGCAGAGCmψmψCmψACAGAAAGAAGCmψGAAGGAGGmψ | |
| | GmψGGAAGCCmψGCCGmψGGGAAGCCCCGCmψGCCAAGAGAGmψGAAGCmψGGAC | |
| | mψGAAmψAAAGCCmψGAGmyAGGAAGmψCmψAGAAAAAAAAAAAAAAAAAAAAAA | |
| | GCCCmψmψCmψmψCmψCmψCCCmψmψGCACCmψGmψACCmψCmψmψGGmψCmψmψ | |
| | mψGAAmψAAAGCCmψGAGmψAGGAAGmψCmψAGAAAAAAAAAAAAAAAAAAAAAA | |
| | AAAAAAAAAAAAAAAAAAAAAAAAAAAAAAAAAAAAAAAAAAAAAAAAAAAAAAA | |
| | AAA | |

Synthesis of gRNAs:

In this example, gRNAs targeting the mouse PCSK9 locus were designed using gRNA scaffold 174 with a v1 modification profile (see Example 5) and chemically synthesized. The sequences of the PCSK9-targeting spacers are listed in Table 22.

TABLE 22
Sequences of spacers targeting the mouse PCSK9 locus assayed in
this example

| Spacer ID | Target | RNA sequence | SEQ ID NO |
|---|---|---|---|
| 27.103 | mouse PCSK9 | UAAUCUCCAUCCUCGUCCUG | 19744 |
| 27.105 | mouse PCSK9 | CCAAGAAGCCAGGGAAGAGG | 19745 |
| 27.106 | mouse PCSK9 | ACAUAUCUUUUAUGACCUCU | 19746 |
| 27.107 | mouse PCSK9 | CUGGCUUCUUGGUGAAGAUG | 19747 |
| 27.108 | mouse PCSK9 | UGGUGAAGAUGAGCAGUGAC | 19748 |
| 27.116 | mouse PCSK9 | GCCGUUGCUCCAAGGUAUGG | 19749 |
| 27.117 | mouse PCSK9 | UUCUUGGGGAUCAGGAGGCC | 19750 |

Transfection of CasX mRNA and gRNA into Mouse Hepal-6 Cells In Vitro:

Editing at the mouse PCSK9 locus was assessed by delivering in vitro transcribed CasX mRNA (CasX mRNA #1 or CasX mRNA #2; see Table 20) and synthesized gRNAs targeting PCSK9 into Hepal-6 cells via transfection. Briefly, each well of 20,000 Hepal-6 cells were lipofected with in vitro transcribed mRNA coding for CasX 676 and a PCSK9-targeting gRNA. After a media change, transfected cells were harvested at 20 hours post-transfection for editing assessment at the PCSK9 locus by next-generation sequencing (NGS). As experimental controls, individual transfections of CasX mRNA #1 and CasX mRNA #2 without gRNAs were performed.

### NGS Processing and Analysis:

Genomic DNA (gDNA) from harvested cells were extracted using the Zymo Quick-DNA Miniprep Plus kit following the manufacturer's instructions. Target amplicons were formed by amplifying regions of interest from 50-100 ng of extracted gDNA with a set of primers targeting the human PCSK9 locus. These gene-specific primers contained an additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 (v 2.0.29) program. The percentage of reads modified in a window around the 3′ end of the spacer was determined. The activity of the CasX molecule was quantified as the total percent of reads that contain insertions, substitutions, and/or deletions anywhere within this window for each.

### Results

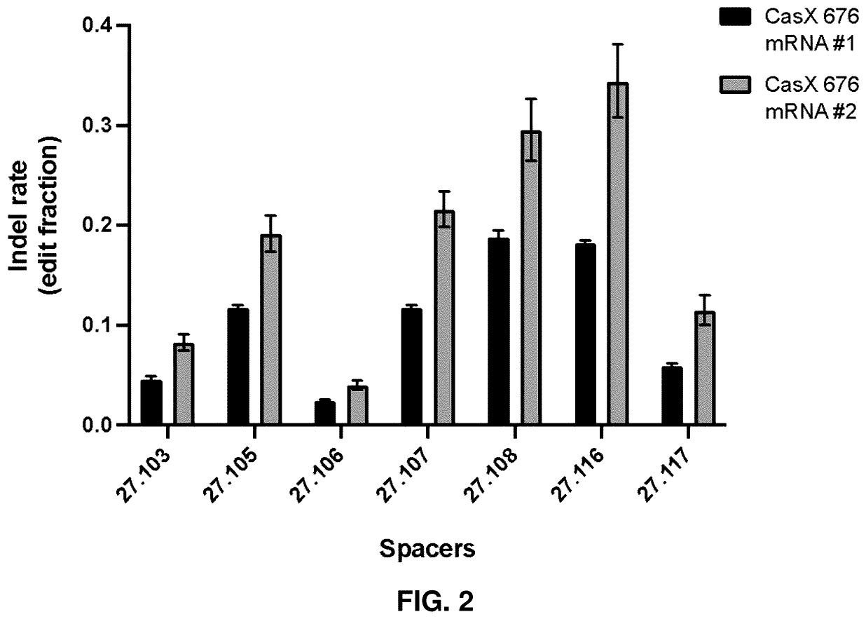

CasX-mediated editing at the mouse PCSK9 locus was used to evaluate the effects of incorporating different 5′ and 3′ UTRs into the engineered CasX mRNA. The plot in FIG. 2 shows the quantification of percent editing measured as indel rate at the PCSK9 locus in mouse Hepal-6 cells transfected with CasX 676 mRNA #1 or CasX 676 mRNA #2 with the indicated PCSK9-targeting gRNAs. The data demonstrate that for all targeting spacers tested in this experiment, CasX mRNA #2, the mRNA with a synthetic 5′ UTR and a mouse HBA 3′ UTR, consistently exhibited higher editing levels at the mouse PCSK9 locus compared to editing levels achieved by CasX mRNA #1, the mRNA with human HBA 5′ and 3′ UTRs. Specifically, the highest level of editing rate achieved was with spacer 27.116, where use of CasX mRNA #2 resulted in ˜35% editing efficiency compared to ˜20% editing level by CasX mRNA #1 (FIG. 2).

The results demonstrate that altering the 5′UTR and 3′UTR sequences of the CasX mRNA can affect the editing activity of CasX at a target locus in a cell-based assay, and that the combination of a synthetic 5′ UTR and mouse HBA 3′ UTR was particularly effective for generating an mRNA encoding CasX that produced a high level of editing.

### Example 6: CasX mRNA and PCSK9-Targeting gRNA can be Delivered Via LNPs to Achieve Editing at the Human PCSK9 Locus In Vitro

Experiments were performed to demonstrate that delivery of LNPs encapsulating CasX mRNA and a PCSK9-targeting gRNA can induce editing at the endogenous human PCSK9 locus in primary human hepatocytes. Here, CasX 515 was selected for assessment given its improvement in specificity while maintaining activity compared to the earlier prototype CasX 491, and CasX 812 was selected given its increased specificity. Materials and Methods:

Generation of CasX mRNAs encoding for CasX 515 and CasX 812 was performed by IVT, following similar methods described earlier in Example 5. Briefly, constructs encoding for a synthetic 5′UTR, a codon-optimized CasX 515 or CasX 812 with flanking c-MYC NLSes, and a 3′UTR derived from the mouse hemoglobin alpha (mHBA) were cloned into a plasmid containing a T7 promoter and 79-nucleotide poly (A) tail. The resulting plasmid was linearized prior to use for IVT reactions, which were carried out with CleanCap® AG and NI-methyl-pseudouridine (as described in Example 5). The DNA sequences encoding the CasX 515 or CasX 812 mRNA molecules are listed in Table 23, with the corresponding mRNA sequences with the chemical modifications listed in Table 24. The protein sequences for CasX 515 and CasX 812 resulting from expression of the IVT mRNA molecules are listed in Table 25.

TABLE 23
Encoding sequences of the two CasX mRNA molecules assessed in this
example*.

| CasX | Component | | | SEQ ID |
|---|---|---|---|---|
| mRNA ID | (ID) | Description | DNA sequence | NO |
| CasX 515 | 5′UTR + | Synthetic | AAATAAGAGAGAAAAGAAGAGTAAGAAGAAATATAA | 19739 |
| & | Kozak | | GAGCCACC | |
| CasX 812 | sequence | | | |
| mRNA | (underlined) | | | |
| | START | | ATGGCCCCTGCTGCCAAGAGAGTGAAGCTGGATAGC | 19734 |
| | codon + c- | | AGA | |
| | MYC NLS | | | |
| CasX 515 | CasX 515 | | CAGGAGATCAAGCGGATTAATAAAATTCGGAGAAGA | 19751 |
| mRNA | | | CTGGTGAAGGATTCTAACACAAAGAAGGCTGGCAAG | |
| | | | ACAGGCCCTATGAAGACACTGCTGGTGAGAGTGATG | |
| | | | ACACCCGACCTGAGAGAAAGACTGGAAAACCTGAGA | |
| | | | AAGAAGCCTGAGAATATCCCCCAGCCCATCAGCAAC | |
| | | | ACAAGCCGGGCCAACCTGAATAAGCTGCTGACCGAC | |
| | | | TACACCGAAATGAAGAAGGCCATCCTGCACGTGTAT | |
| | | | TGGGAAGAGTTCCAGAAAGACCCAGTCGGCCTGATG | |
| | | | AGCAGAGTGGCTCAGCCTGCCAGCAAGAAGATCGAT | |
| | | | CAGAACAAGCTGAAGCCCGAAATGGACGAGAAGGGG | |
| | | | AACCTGACAACCGCCGGCTTTGCCTGTAGCCAGTGC | |
| | | | GGCCAGCCCCTGTTTGTGTACAAACTGGAACAGGTG | |
| | | | AGCGAAAAGGGCAAGGCTTACACGAATTACTTCGGC | |
| | | | AGATGCAACGTGGCCGAGCACGAGAAGCTGATCCTG | |
| | | | CTGGCCCAGCTGAAGCCTGAGAAGGATAGCGATGAG | |
| | | | GCAGTGACATATTCCCTGGGCAAGTTCGGACAGCGG | |
| | | | GCCCTGGATTTTTATTCCATTCATGTGACCAAGGAA | |
| | | | TCCACCCACCCCGTCAAGCCTCTTGCCCAAATTGCC | |
| | | | GGCAACAGATACGCCAGCGGCCCCGTGGGCAAGGCC | |
| | | | CTGAGCGACGCCTGTATGGGCACCATCGCCAGCTTC | |
| | | | CTGTCTAAGTACCAGGACATTATCATCGAGCACCAG | |
| | | | AAGGTGGTGAAGGGCAACCAGAAGAGACTGGAGAGC | |
| | | | CTGCGCGAGCTGGCCGGCAAGGAAAACCTGGAGTAT | |
| | | | CCTAGCGTGACCCTGCCTCCTCAGCCTCATACAAAG | |
| | | | GAGGGCGTGGATGCCTACAACGAAGTGATCGCCCGG | |
| | | | GTGCGGATGTGGGTGAACCTGAATCTGTGGCAGAAG | |
| | | | CTGAAGCTGTCTAGAGACGACGCCAAGCCCCTGCTG | |
| | | | AGACTGAAGGGCTTCCCCAGCTTCCCTCTGGTGGAG | |
| | | | AGACAGGCAAATGAAGTGGACTGGTGGGACATGGTG | |
| | | | TGTAACGTGAAGAAGCTGATCAATGAGAAGAAGGAG | |
| | | | GACGGCAAAGTGTTCTGGCAGAATCTGGCCGGCTAC | |
| | | | AAGCGTCAGGAGGCCCTGCGGCCCTACCTGAGCAGC | |
| | | | GAGGAAGACAGAAAGAAGGGCAAGAAGTTCGCCCGG | |
| | | | TATCAGCTGGGGGACCTGCTGCTGCACCTCGAGAAG | |
| | | | AAGCACGGCGAAGACTGGGGGAAGGTGTACGATGAG | |
| | | | GCCTGGGAGCGGATCGATAAGAAGGTGGAGGGCCTG | |
| | | | AGCAAGCACATCAAGCTGGAGGAGGAACGGAGATCT | |
| | | | GAGGACGCCCAGAGCAAGGCCGCCCTGACCGACTGG | |
| | | | CTGAGAGCCAAGGCCAGCTTCGTCATCGAGGGGCTG | |
| | | | AAGGAGGCCGACAAGGACGAGTTCTGCCGGTGCGAA | |
| | | | CTGAAGCTGCAGAAGTGGTACGGAGATCTGAGAGGC | |
| | | | AAACCTTTCGCCATCGAGGCCGAGAACAGCATCCTG | |
| | | | GACATCAGCGGCTTCAGCAAGCAGTACAACTGCGCC | |
| | | | TTTATTTGGCAGAAGGACGGAGTGAAGAAGCTGAAC | |
| | | | CTGTACCTGATCATCAACTATTTCAAGGGCGGCAAG | |
| | | | CTGAGATTCAAGAAGATCAAGCCTGAAGCCTTCGAG | |
| | | | GCCAACAGATTCTACACCGTGATTAACAAGAAAAGC | |
| | | | GGAGAGATCGTGCCAATGGAAGTGAACTTCAACTTC | |
| | | | GACGACCCTAACCTGATCATCCTGCCCCTGGCATTT | |
| | | | GGCAAGCGGCAGGGCAGAGAGTTCATCTGGAACGAC | |
| | | | CTGCTGTCTCTGGAGACCGGCAGCCTGAAGCTGGCC | |
| | | | AACGGCAGAGTGATCGAGAAGACACTGTACAACAGA | |
| | | | CGAACCAGACAAGACGAGCCCGCCCTGTTTGTGGCC | |
| | | | CTGACCTTCGAGAGAAGAGAGGTGCTGGACAGCAGC | |
| | | | AATATCAAGCCTATGAACCTGATCGGCGTGGACCGG | |
| | | | GGCGAGAACATCCCTGCCGTGATCGCCCTTACCGAC | |
| | | | CCCGAGGGATGCCCTCTGAGCCGGTTTAAAGACAGC | |
| | | | CTGGGCAACCCTACCCACATCCTGAGAATTGGCGAG | |
| | | | TCCTACAAGGAGAAGCAGAGAACCATCCAGGCCAAG | |
| | | | AAGGAGGTGGAGCAGCGGCGGGCTGGCGGCTACTCC | |
| | | | CGGAAGTACGCCAGCAAGGCCAAGAACCTGGCCGAC | |
| | | | GACATGGTTAGAAATACCGCCAGAGACCTCCTGTAC | |
| | | | TACGCTGTGACCCAGGACGCCATGCTGATCTTCGAG | |
| | | | AACCTGAGCAGAGGCTTCGGCAGACAGGGCAAGAGA | |
| | | | ACCTTCATGGCCGAGAGACAGTACACCCGGATGGAG | |
| | | | GACTGGCTGACCGCCAAGCTGGCCTACGAGGGCCTG | |
| | | | CCCTCTAAGACCTACCTGTCCAAGACCTTGGCACAG | |
| | | | TACACCAGCAAGACATGCTCTAACTGCGGCTTCACA | |
| | | | ATCACGAGCGCCGACTACGACCGGGTGCTGGAGAAA | |
| | | | CTGAAGAAGACCGCCACAGGCTGGATGACCACCATT | |
| | | | AACGGCAAGGAGCTGAAGGTGGAGGGCCAGATCACC | |
| | | | TACTACAACAGGTACAAACGGCAGAACGTGGTGAAG | |
| | | | GACCTGAGCGTGGAACTGGATAGACTGAGCGAGGAA | |
| | | | AGCGTAAACAATGACATCAGCAGCTGGACCAAGGGC | |
| | | | CGGAGCGGCGAGGCCCTGAGCCTGCTGAAGAAGAGA | |
| | | | TTCTCCCACAGACCAGTGCAGGAGAAGTTCGTGTGT | |
| | | | CTGAACTGCGGCTTCGAGACCCACGCCGACGAGCAA | |
| | | | GCCGCCCTGAACATCGCCCGGTCTTGGCTTTTCCTG | |
| | | | CGGAGCCAGGAGTACAAGAAGTACCAGACAAACAAG | |
| | | | ACCACAGGCAACACAGACAAGAGAGCCTTCGTCGAG | |
| | | | ACCTGGCAGAGCTTCTACAGAAAGAAGCTGAAGGAG | |
| | | | GTGTGGAAGCCTGCCGTG | |
| CasX 812 | CasX 812 | | CAGGAGATCAAGCGGATTAATAAAATTCGGAGAAGA | 19752 |
| mRNA | | | CTGGTGAAGGATTCTAACACAAAGAAGGCTGGCAAG | |
| | | | ACAGGCCCTATGAAGACACTGCTGGTGAGAGTGATG | |
| | | | ACACCCGACCTGAGAGAAAGACTGGAAAACCTGAGA | |
| | | | AAGAAGCCTGAGAATATCCCCCAGCCCATCAGCAAC | |
| | | | ACAAGCCGGGCCAACCTGAATAAGCTGCTGACCGAC | |
| | | | TACACCGAAATGAAGAAGGCCATCCTGCACGTGTAT | |
| | | | TGGGAAGAGTTCCAGAAAGACCCAGTCGGCCTGATG | |
| | | | AGCAGAGTGGCTCAGCCTGCCAGCAAGAAGATCGAT | |
| | | | CAGAACAAGCTGAAGCCCGAAATGGACGAGAAGGGG | |
| | | | AACCTGACAACCGCCGGCTTTGCCTGTAGCCAGTGC | |
| | | | GGCCAGCCCCTGTTTGTGTACAAACTGGAACAGGTG | |
| | | | AGCGAAAAGGGCAAGGCTTACACGAATTACTTCGGC | |
| | | | AGATGCAACGTGGCCGAGCACGAGAAGCTGATCCTG | |
| | | | CTGGCCCAGCTGAAGCCTGAGAAGGATAGCGATGAG | |
| | | | GCAGTGACATATTCCCTGGGCAAGTTCGGACAGCGG | |
| | | | GCCCTGGATTTTTATTCCATTCATGTGACCAAGGAA | |
| | | | TCCACCCACCCCGTCAAGCCTCTTGCCCAAATTGCC | |
| | | | GGCAACAGATACGCCAGCGGCCCCGTGGGCAAGGCC | |
| | | | CTGAGCGACGCCTGTATGGGCACCATCGCCAGCTTC | |
| | | | CTGTCTAAGTACCAGGACATTATCATCGAGCACCAG | |
| | | | AAGGTGGTGAAGGGCAACCAGAAGAGACTGGAGAGC | |
| | | | CTGCGCGAGCTGGCCGGCAAGGAAAACCTGGAGTAT | |
| | | | CCTAGCGTGACCCTGCCTCCTCAGCCTCATACAAAG | |
| | | | GAGGGCGTGGATGCCTACAACGAAGTGATCGCCCGG | |
| | | | GTGCGGATGTGGGTGAACCTGAATCTGTGGCAGAAG | |
| | | | CTGAAGCTGTCTAGAGACGACGCCAAGCCCCTGCTG | |
| | | | AGACTGAAGAAGTTCCCCAGCTTCCCTCTGGTGGAG | |
| | | | AGACAGGCAAATGAAGTGGACTGGTGGGACATGGTG | |
| | | | TGTAACGTGAAGAAGCTGATCAATGAGAAGAAGGAG | |
| | | | GACGGCAAAGTGTTCTGGCAGAATCTGGCCGGCTAC | |
| | | | AAGCGTCAGGAGGCCCTGCGGCCCTACCTGAGCAGC | |
| | | | GAGGAAGACAGAAAGAAGGGCAAGAAGTTCGCCCGG | |
| | | | TATCAGCTGGGGGACCTGCTGCTGCACCTCGAGAAG | |
| | | | AAGCACGGCGAAGACTGGGGGAAGGTGTACGATGAG | |
| | | | GCCTGGGAGCGGATCGATAAGAAGGTGGAGGGCCTG | |
| | | | AGCAAGCACATCAAGCTGGAGGAGGAACGGAGATCT | |
| | | | GAGGACGCCCAGAGCAAGGCCGCCCTGACCGACTGG | |
| | | | CTGAGAGCCAAGGCCAGCTTCGTCATCGAGGGGCTG | |
| | | | AAGGAGGCCGACAAGGACGAGTTCTGCCGGTGCGAA | |
| | | | CTGAAGCTGCAGAAGTGGTACGGAGATCTGAGAGGC | |
| | | | AAACCTTTCGCCATCGAGGCCGAGAACAGCATCCTG | |
| | | | GACATCAGCGGCTTCAGCAAGCAGTACAACTGCGCC | |
| | | | TTTATTTGGCAGAAGGACGGAGTGAAGAAGCTGAAC | |
| | | | CTGTACCTGATCATCAACTATTTCAAGGGCGGCAAG | |
| | | | CTGAGATTCAAGAAGATCAAGCCTGAAGCCTTCGAG | |
| | | | GCCAACAGATTCTACACCGTGATTAACAAGAAAAGC | |
| | | | GGAGAGATCGTGCCAATGGAAGTGAACTTCAACTTC | |
| | | | GACGACCCTAACCTGATCATCCTGCCCCTGGCATTT | |
| | | | GGCAAGCGGCAGGGCAGAGAGTTCATCTGGAACGAC | |
| | | | CTGCTGTCTCTGGAGACCGGCAGCCTGAAGCTGGCC | |
| | | | AACGGCAGAGTGATCGAGAAGACACTGTACAACAGA | |
| | | | CGAACCAGACAAGACGAGCCCGCCCTGTTTGTGGCC | |
| | | | CTGACCTTCGAGAGAAGAGAGGTGCTGGACAGCAGC | |
| | | | AATATCAAGCCTATGAACCTGATCGGCGTGGACCGG | |
| | | | GGCGAGAACATCCCTGCCGTGATCGCCCTTACCGAC | |
| | | | CCCGAGGGATGCCCTCTGAGCCGGTTTAAAGACAGC | |
| | | | CTGGGCAACCCTACCCACATCCTGAGAATTGGCGAG | |
| | | | TCCTACAAGGAGAAGCAGAGAACCATCCAGGCCAAG | |
| | | | AAGGAGGTGGAGCAGCGGCGGGCTGGCGGCTACTCC | |
| | | | CGGAAGTACGCCAGCAAGGCCAAGAACCTGGCCGAC | |
| | | | GACATGGTTAGAAATACCGCCAGAGACCTCCTGTAC | |
| | | | TACGCTGTGACCCAGGACGCCATGCTGATCTTCGAG | |
| | | | AACCTGAGCAGAGGCTTCGGCAGACAGGGCAAGAGA | |
| | | | ACCTTCATGGCCGAGAGACAGTACACCCGGATGGAG | |
| | | | GACTGGCTGACCGCCAAGCTGGCCTACGAGGGCCTG | |
| | | | CCCTCTAAGACCTACCTGTCCAAGACCTTGGCACAG | |
| | | | TACACCAGCAAGACATGCTCTAACTGCGGCTTCACA | |
| | | | ATCACGAGCGCCGACTACGACCGGGTGCTGGAGAAA | |
| | | | CTGAAGAAGACCGCCACAGGCTGGATGACCACCATT | |
| | | | AACGGCAAGGAGCTGAAGGTGGAGGGCCAGATCACC | |
| | | | TACTACAACAGGTACAAACGGCAGAACGTGGTGAAG | |
| | | | GACCTGAGCGTGGAACTGGATAGACTGAGCGAGGAA | |
| | | | AGCGTAAACAATGACATCAGCAGCTGGACCAAGGGC | |
| | | | CGGAGCGGCGAGGCCCTGAGCCTGCTGAAGAAGAGA | |
| | | | TTCTCCCACAGACCAGTGCAGGAGAAGTTCGTGTGT | |
| | | | CTGAACTGCGGCTTCGAGACCCACGCCGACGAGCAA | |
| | | | GCCGCCCTGAACATCGCCCGGTCTTGGCTTTTCCTG | |
| | | | CGGAGCCAGGAGTACAAGAAGTACCAGACAAACAAG | |
| | | | ACCACAGGCAACACAGACAAGAGAGCCTTCGTCGAG | |
| | | | ACCTGGCAGAGCTTCTACAGAAAGAAGCTGAAGGAG | |
| | | | GTGTGGAAGCCTGCCGTG | |
| CasX 515 | c-MYC NLS + | | GGAAGCCCCGCTGCCAAGAGAGTGAAGCTGGACTAA | 19753 |
| & | STOP codon | | | |
| CasX 812 | 3′UTR | Mouse HBA | GCTGCCTTCTGCGGGGCTTGCCTTCTGGCCATGCCC | 19740 |
| mRNA | | | TTCTTCTCTCCCTTGCACCTGTACCTCTTGGTCTTT | |
| | | | GAATAAAGCCTGAGTAGGAAG | |
| | XbaI | | TCTAG | 19741 |
| | restriction | | | |
| | site (partial) | | | |
| | Poly(A) tail | | AAAAAAAAAAAAAAAAAAAAAAAAAAAAAAAAAAAA | 19826 |
| | | | AAAAAAAAAAAAAAAAAAAAAAAAAAAAAAAAAAAA | |
| | | | AAAAAAA | |

*Components are listed in a 5′ to 3′ order within the constructs

TABLE 24
Full-length RNA sequences of CasX mRNA molecules assessed in this
example. The CleanCap ® AG 5′ cap is not shown in the table. Modification ‘mψ’ = N1-
methyl-pseudouridine

| CasX | | SEQ ID |
|---|---|---|
| mRNA | RNA sequence | NO |
| CasX 515 | AAAmψAAGAGAGAAAAGAAGAGmψAAGAAGAAAmψAmψAAGAGCCACCAmψGGCCCCm | 19755 |
| mRNA | ψGCmψGCCAAGAGAGmψGAAGCmψGGAmψAGCAGACAGGAGAmψCAAGCGGAmψmψAA | |
| | mψAAAAmψmψCGGAGAAGACmψGGmψGAAGGAmψmψCmψAACACAAAGAAGGCmψGGC | |
| | AAGACAGGCCCmψAmψGAAGACACmψGCmψGGmψGAGAGmψGAmψGACACCCGACCmψ | |
| | GAGAGAAAGACmψGGAAAACCmψGAGAAAGAAGCCmψGAGAAmψAmψCCCCCAGCCCA | |
| | mψCAGCAACACAAGCCGGGCCAACCmψGAAmψAAGCmψGCmψGACCGACmψACACCGA | |
| | AAmψGAAGAAGGCCAmψCCmψGCACGmψGmψAmψmψGGGAAGAGmψmψCCAGAAAGAC | |
| | CCAGmψCGGCCmψGAmψGAGCAGAGmψGGCmψCAGCCmψGCCAGCAAGAAGAmψCGAm | |
| | ψCAGAACAAGCmψGAAGCCCGAAAmψGGACGAGAAGGGGAACCmψGACAACCGCCGGC | |
| | mψmψmψGCCmψGmψAGCCAGmψGCGGCCAGCCCCmψGmψmψmψGmψGmψACAAACmψG | |
| | GAACAGGmψGAGCGAAAAGGGCAAGGCmψmψACACGAAmψmψACmψmψCGGCAGAmψG | |
| | CAACGmψGGCCGAGCACGAGAAGCmψGAmψCCmψGCmψGGCCCAGCmψGAAGCCmψGA | |
| | GAAGGAmψAGCGAmψGAGGCAGmψGACAmψAmψmψCCCmψGGGCAAGmψmψCGGACAG | |
| | CGGGCCCmψGGAmψmψmψmψmψAmψmψCCAmψmψCAmψGmψGACCAAGGAAmψCCACC | |
| | CACCCCGmψCAAGCCmψCmψmψGCCCAAAmψmψGCCGGCAACAGAmψACGCCAGCGGC | |
| | CCCGmψGGGCAAGGCCCmψGAGCGACGCCmψGmψAmψGGGCACCAmψCGCCAGCmψmψ | |
| | CCmψGmψCmψAAGmψACCAGGACAmψmψAmψCAmψCGAGCACCAGAAGGmψGGmψGAA | |
| | GGGCAACCAGAAGAGACmψGGAGAGCCmψGCGCGAGCmψGGCCGGCAAGGAAAACCmψ | |
| | GGAGmψAmψCCmψAGCGmψGACCCmψGCCmψCCmψCAGCCmψCAmψACAAAGGAGGGC | |
| | GmψGGAmψGCCmψACAACGAAGmψGAmψCGCCCGGGmψGCGGAmψGmψGGGmψGAACC | |
| | mψGAAmψCmψGmψGGCAGAAGCmψGAAGCmψGmψCmψAGAGACGACGCCAAGCCCCmψ | |
| | GCmψGAGACmψGAAGGGCmψmψCCCCAGCmψmψCCCmψCmψGGmψGGAGAGACAGGCA | |
| | AAmψGAAGmψGGACmψGGmψGGGACAmψGGmψGmψGmψAACGmψGAAGAAGCmψGAmψ | |
| | CAAmψGAGAAGAAGGAGGACGGCAAAGmψGmψmψCmψGGCAGAAmψCmψGGCCGGCmψ | |
| | ACAAGCGmψCAGGAGGCCCmψGCGGCCCmψACCmψGAGCAGCGAGGAAGACAGAAAGA | |
| | AGGGCAAGAAGmψmψCGCCCGGmψAmψCAGCmψGGGGGACCmψGCmψGCmψGCACCmψ | |
| | CGAGAAGAAGCACGGCGAAGACmψGGGGGAAGGmψGmψACGAmψGAGGCCmψGGGAGC | |
| | GGAmψCGAmψAAGAAGGmψGGAGGGCCmψGAGCAAGCACAmψCAAGCmψGGAGGAGGA | |
| | ACGGAGAmψCmψGAGGACGCCCAGAGCAAGGCCGCCCmψGACCGACmψGGCmψGAGAG | |
| | CCAAGGCCAGCmψmψCGmψCAmψCGAGGGGCmψGAAGGAGGCCGACAAGGACGAGmψm | |
| | ψCmψGCCGGmψGCGAACmψGAAGCmψGCAGAAGmψGGmψACGGAGAmψCmψGAGAGGC | |
| | AAACCmψmψmψCGCCAmψCGAGGCCGAGAACAGCAmψCCmyGGACAmψCAGCGGCmψm | |
| | ψCAGCAAGCAGmψACAACmψGCGCCmψmψmψAmψmψmψGGCAGAAGGACGGAGmψGAA | |
| | GAAGCmψGAACCmψGmψACCmψGAmψCAmψCAACmψAmψmψmψCAAGGGCGGCAAGCm | |
| | ψGAGAmψmψCAAGAAGAmψCAAGCCmψGAAGCCmψmψCGAGGCCAACAGAmψmψCmψA | |
| | CACCGmψGAmψmψAACAAGAAAAGCGGAGAGAmψCGmψGCCAAmψGGAAGmψGAACmψ | |
| | mψCAACmψmψCGACGACCCmψAACCmψGAmψCAmψCCmψGCCCCmψGGCAmψmψmψGG | |
| | CAAGCGGCAGGGCAGAGAGmψmψCAmψCmψGGAACGACCmψGCmψGmψCmψCmψGGAG | |
| | ACCGGCAGCCmψGAAGCmψGGCCAACGGCAGAGmψGAmψCGAGAAGACACmψGmψACA | |
| | ACAGACGAACCAGACAAGACGAGCCCGCCCmψGmψmψmψGmψGGCCCmψGACCmψmψC | |
| | GAGAGAAGAGAGGmψGCmψGGACAGCAGCAAmψAmψCAAGCCmψAmψGAACCmψGAmψ | |
| | CGGCGmψGGACCGGGGCGAGAACAmψCCCmψGCCGmψGAmψCGCCCmψmψACCGACCC | |
| | CGAGGGAmψGCCCmψCmψGAGCCGGmψmψmψAAAGACAGCCmψGGGCAACCCmψACCC | |
| | ACAmψCCmyGAGAAmψmψGGCGAGmψCCmψACAAGGAGAAGCAGAGAACCAmψCCAGG | |
| | CCAAGAAGGAGGmψGGAGCAGCGGCGGGCmψGGCGGCmψACmψCCCGGAAGmψACGCC | |
| | AGCAAGGCCAAGAACCmψGGCCGACGACAmψGGmψmψAGAAAmψACCGCCAGAGACCm | |
| | ψCCmψGmψACmψACGCmψGmψGACCCAGGACGCCAmψGCmψGAmψCmψmψCGAGAACC | |
| | mψGAGCAGAGGCmψmψCGGCAGACAGGGCAAGAGAACCmψmψCAmψGGCCGAGAGACA | |
| | GmψACACCCGGAmψGGAGGACmψGGCmψGACCGCCAAGCmψGGCCmψACGAGGGCCmψ | |
| | GCCCmψCmψAAGACCmψACCmψGmψCCAAGACCmψmψGGCACAGmψACACCAGCAAGA | |
| | CAmψGCmψCmψAACmψGCGGCmψmψCACAAmψCACGAGCGCCGACmψACGACCGGGmψ | |
| | GCmψGGAGAAACmψGAAGAAGACCGCCACAGGCmψGGAmψGACCACCAmψmψAACGGC | |
| | AAGGAGCmψGAAGGmψGGAGGGCCAGAmψCACCmψACmψACAACAGGmψACAAACGGC | |
| | AGAACGmψGGmψGAAGGACCmψGAGCGmψGGAACmψGGAmψAGACmψGAGCGAGGAAA | |
| | GCGmψAAACAAmψGACAmψCAGCAGCmψGGACCAAGGGCCGGAGCGGCGAGGCCCmψG | |
| | AGCCmψGCmψGAAGAAGAGAmψmψCmψCCCACAGACCAGmψGCAGGAGAAGmψmψCGm | |
| | ψGmψGmψCmψGAACmψGCGGCmψmψCGAGACCCACGCCGACGAGCAAGCCGCCCmψGA | |
| | ACAmψCGCCCGGmψCmψmψGGCmψmψmψmψCCmψGCGGAGCCAGGAGmψACAAGAAGm | |
| | ψACCAGACAAACAAGACCACAGGCAACACAGACAAGAGAGCCmψmψCGmψCGAGACCm | |
| | ψGGCAGAGCmψmψCmψACAGAAAGAAGCmψGAAGGAGGmψGmψGGAAGCCmψGCCGmψ | |
| | GGGAAGCCCCGCmψGCCAAGAGAGmψGAAGCmψGGACmψAAGCmψGCCmψmψCmψGCG | |
| | GGGCmψmψGCCmψmψCmψGGCCAmψGCCCmψmψCmψmψCmψCmψCCCmψmψGCACCmψ | |
| | GmψACCmψCmψmψGGmψCmψmψmψGAAmψAAAGCCmψGAGmψAGGAAGmψCmψAGAAA | |
| | AAAAAAAAAAAAAAAAAAAAAAAAAAAAAAAAAAAAAAAAAAAAAAAAAAAAAAAAAA | |
| | AAAAAAAAAAAAAAAAAA | |
| CasX 812 | AAAmψAAGAGAGAAAAGAAGAGmψAAGAAGAAAmψAmψAAGAGCCACCAmψGGCCCCm | 19756 |
| mRNA | ψGCmψGCCAAGAGAGmψGAAGCmψGGAmψAGCAGACAGGAGAmψCAAGCGGAmψmψAA | |
| | mψAAAAmψmψCGGAGAAGACmψGGmψGAAGGAmψmψCmψAACACAAAGAAGGCmψGGC | |
| | AAGACAGGCCCmψAmψGAAGACACmψGCmψGGmψGAGAGmψGAmψGACACCCGACCmψ | |
| | GAGAGAAAGACmψGGAAAACCmψGAGAAAGAAGCCmψGAGAAmψAmψCCCCCAGCCCA | |
| | mψCAGCAACACAAGCCGGGCCAACCmψGAAmψAAGCmψGCmψGACCGACmψACACCGA | |
| | AAmψGAAGAAGGCCAmψCCmψGCACGmψGmψAmψmψGGGAAGAGmψmψCCAGAAAGAC | |
| | CCAGmψCGGCCmψGAmψGAGCAGAGmψGGCmψCAGCCmψGCCAGCAAGAAGAmψCGAm | |
| | ψCAGAACAAGCmψGAAGCCCGAAAmψGGACGAGAAGGGGAACCmψGACAACCGCCGGC | |
| | mψmψmψGCCmψGmψAGCCAGmψGCGGCCAGCCCCmψGmψmψmψGmψGmψACAAACmψG | |
| | GAACAGGmψGAGCGAAAAGGGCAAGGCmψmψACACGAAmψmψACmψmψCGGCAGAmψG | |
| | CAACGmψGGCCGAGCACGAGAAGCmψGAmψCCmψGCmψGGCCCAGCmψGAAGCCmψGA | |
| | GAAGGAmψAGCGAmψGAGGCAGmψGACAmψAmψmψCCCmψGGGCAAGmψmψCGGACAG | |
| | CGGGCCCmψGGAmψmψmψmψmψAmψmψCCAmψmψCAmψGmψGACCAAGGAAmψCCACC | |
| | CACCCCGmψCAAGCCmψCmψmψGCCCAAAmψmψGCCGGCAACAGAmψACGCCAGCGGC | |
| | CCCGmψGGGCAAGGCCCmψGAGCGACGCCmψGmψAmψGGGCACCAmψCGCCAGCmψmψ | |
| | CCmψGmψCmψAAGmψACCAGGACAmψmψAmψCAmψCGAGCACCAGAAGGmψGGmψGAA | |
| | GGGCAACCAGAAGAGACmψGGAGAGCCmψGCGCGAGCmψGGCCGGCAAGGAAAACCmψ | |
| | GGAGmψAmψCCmψAGCGmψGACCCmψGCCmψCCmψCAGCCmψCAmψACAAAGGAGGGC | |
| | GmψGGAmψGCCmψACAACGAAGmψGAmψCGCCCGGGmψGCGGAmψGmψGGGmψGAACC | |
| | mψGAAmψCmψGmψGGCAGAAGCmψGAAGCmψGmψCmψAGAGACGACGCCAAGCCCCmψ | |
| | GCmψGAGACmψGAAGAAGmψmψCCCCAGCmψmψCCCmψCmψGGmψGGAGAGACAGGCA | |
| | AAmψGAAGmψGGACmψGGmψGGGACAmψGGmψGmψGmψAACGmψGAAGAAGCmψGAmψ | |
| | CAAmψGAGAAGAAGGAGGACGGCAAAGmψGmψmψCmψGGCAGAAmψCmψGGCCGGCmψ | |
| | ACAAGCGmψCAGGAGGCCCmψGCGGCCCmψACCmψGAGCAGCGAGGAAGACAGAAAGA | |
| | AGGGCAAGAAGmψmψCGCCCGGmψAmψCAGCmψGGGGGACCmψGCmψGCmψGCACCmψ | |
| | CGAGAAGAAGCACGGCGAAGACmψGGGGGAAGGmψGmψACGAmψGAGGCCmψGGGAGC | |
| | GGAmψCGAmψAAGAAGGmψGGAGGGCCmψGAGCAAGCACAmψCAAGCmψGGAGGAGGA | |
| | ACGGAGAmψCmψGAGGACGCCCAGAGCAAGGCCGCCCmψGACCGACmψGGCmψGAGAG | |
| | CCAAGGCCAGCmψmψCGmψCAmψCGAGGGGCmψGAAGGAGGCCGACAAGGACGAGmψm | |
| | ψCmψGCCGGmψGCGAACmψGAAGCmψGCAGAAGmψGGmψACGGAGAmψCmψGAGAGGC | |
| | AAACCmψmψmψCGCCAmψCGAGGCCGAGAACAGCAmψCCmyGGACAmψCAGCGGCmψm | |
| | ψCAGCAAGCAGmψACAACmψGCGCCmψmψmψAmψmψmψGGCAGAAGGACGGAGmψGAA | |
| | GAAGCmψGAACCmψGmψACCmψGAmψCAmψCAACmψAmψmψmψCAAGGGCGGCAAGCm | |
| | ψGAGAmψmψCAAGAAGAmψCAAGCCmψGAAGCCmψmψCGAGGCCAACAGAmψmψCmψA | |
| | CACCGmψGAmψmψAACAAGAAAAGCGGAGAGAmψCGmψGCCAAmψGGAAGmψGAACmψ | |
| | mψCAACmψmψCGACGACCCmψAACCmψGAmψCAmψCCmψGCCCCmψGGCAmψmψmψGG | |
| | CAAGCGGCAGGGCAGAGAGmψmψCAmψCmψGGAACGACCmψGCmψGmψCmψCmψGGAG | |
| | ACCGGCAGCCmψGAAGCmψGGCCAACGGCAGAGmψGAmψCGAGAAGACACmψGmψACA | |
| | ACAGACGAACCAGACAAGACGAGCCCGCCCmψGmψmψmψGmψGGCCCmψGACCmψmψC | |
| | GAGAGAAGAGAGGmψGCmψGGACAGCAGCAAmψAmψCAAGCCmψAmψGAACCmψGAmψ | |
| | CGGCGmψGGACCGGGGCGAGAACAmψCCCmψGCCGmψGAmψCGCCCmψmψACCGACCC | |
| | CGAGGGAmψGCCCmψCmψGAGCCGGmψmψmψAAAGACAGCCmψGGGCAACCCmψAGGG | |
| | ACAmψCCmψGAGAAmψmψGGCGAGmψCCmψACAAGGAGAAGCAGAGAACCAmψCCAGG | |
| | CCAAGAAGGAGGmψGGAGCAGCGGCGGGCmψGGCGGCmψACmψCCCGGAAGmψACGCC | |
| | AGCAAGGCCAAGAACCmψGGCCGACGACAmψGGmψmψAGAAAmψACCGCCAGAGACCm | |
| | ψCCmψGmψACmψACGCmψGmψGACCCAGGACGCCAmψGCmψGAmψCmψmψCGAGAACC | |
| | mψGAGCAGAGGCmψmψCGGCAGACAGGGCAAGAGAACCmψmψCAmψGGCCGAGAGACA | |
| | GmψACACCCGGAmψGGAGGACmψGGCmψGACCGCCAAGCmψGGCCmψACGAGGGCCmψ | |
| | GCCCmψCmψAAGACCmψACCmψGmψCCAAGACCmψmψGGCACAGmψACACCAGCAAGA | |
| | CAmψGCmψCmψAACmψGCGGCmψmψCACAAmψCACGAGCGCCGACmψACGACCGGGmψ | |
| | GCmψGGAGAAACmψGAAGAAGACCGCCACAGGCmψGGAmψGACCACCAmψmψAACGGC | |
| | AAGGAGCmψGAAGGmψGGAGGGCCAGAmψCACCmψACmψACAACAGGmψACAAACGGC | |
| | AGAACGmψGGmψGAAGGACCmψGAGCGmψGGAACmψGGAmψAGACmψGAGCGAGGAAA | |
| | GCGmψAAACAAmψGACAmψCAGCAGCmψGGACCAAGGGCCGGAGCGGCGAGGCCCmψG | |
| | AGCCmψGCmψGAAGAAGAGAmψmψCmψCCCACAGACCAGmψGCAGGAGAAGmψmψCGm | |
| | ψGmψGmψCmψGAACmψGCGGCmψmψCGAGACCCACGCCGACGAGCAAGCCGCCCmψGA | |
| | ACAmψCGCCCGGmψCmymψGGCmψmψmψmψCCmψGCGGAGCCAGGAGmψACAAGAAGm | |
| | ψGGCAGAGCmψmψCmψACAGAAAGAAGCmψGAAGGAGGmψGmψGGAAGCCmψGCCGmψ | |
| | GGGAAGCCCCGCmψGCCAAGAGAGmψGAAGCmψGGACmψAAGCmψGCCmψmψCmψGCG | |
| | GGGCmψmψGCCmψmψCmψGGCCAmψGCCCmψmψCmψmψCmψCmψCCCmψmψGCACCmψ | |
| | GmψACCmψCmψmψGGmψCmψmψmψGAAmψAAAGCCmψGAGmψAGGAAGmψCmψAGAAA | |
| | AAAAAAAAAAAAAAAAAAAAAAAAAAAAAAAAAAAAAAAAAAAAAAAAAAAAAAAAAA | |
| | AAAAAAAAAAAAAAAAAA | |

TABLE 25
Full-length protein sequences of CasX molecules assessed in this example.

| | | SEQ |
|---|---|---|
| CasX | AA sequence | ID NO |
| CasX 515 | MAPAAKRVKLDSRQEIKRINKIRRRLVKDSNTKKAGKTGPMKTLLVRVMTPDLRERLE | 19757 |
| with | NLRKKPENIPQPISNTSRANLNKLLTDYTEMKKAILHVYWEEFQKDPVGLMSRVAQPA | |
| flanking | SKKIDQNKLKPEMDEKGNLTTAGFACSQCGQPLFVYKLEQVSEKGKAYTNYFGRCNVA | |
| c-MYC | EHEKLILLAQLKPEKDSDEAVTYSLGKFGQRALDFYSIHVTKESTHPVKPLAQIAGNR | |
| NLSes | YASGPVGKALSDACMGTIASFLSKYQDIIIEHQKVVKGNQKRLESLRELAGKENLEYP | |
| | SVTLPPQPHTKEGVDAYNEVIARVRMWVNLNLWQKLKLSRDDAKPLLRLKGFPSFPLV | |
| | ERQANEVDWWDMVCNVKKLINEKKEDGKVFWQNLAGYKRQEALRPYLSSEEDRKKGKK | |
| | FARYQLGDLLLHLEKKHGEDWGKVYDEAWERIDKKVEGLSKHIKLEEERRSEDAQSKA | |
| | ALTDWLRAKASFVIEGLKEADKDEFCRCELKLQKWYGDLRGKPFAIEAENSILDISGF | |
| | SKQYNCAFIWQKDGVKKLNLYLIINYFKGGKLRFKKIKPEAFEANRFYTVINKKSGEI | |
| | VPMEVNFNFDDPNLIILPLAFGKRQGREFIWNDLLSLETGSLKLANGRVIEKTLYNRR | |
| | TRQDEPALFVALTFERREVLDSSNIKPMNLIGVDRGENIPAVIALTDPEGCPLSRFKD | |
| | SLGNPTHILRIGESYKEKQRTIQAKKEVEQRRAGGYSRKYASKAKNLADDMVRNTARD | |
| | LLYYAVTQDAMLIFENLSRGFGRQGKRTFMAERQYTRMEDWLTAKLAYEGLPSKTYLS | |
| | KTLAQYTSKTCSNCGFTITSADYDRVLEKLKKTATGWMTTINGKELKVEGQITYYNRY | |
| | KRQNVVKDLSVELDRLSEESVNNDISSWTKGRSGEALSLLKKRFSHRPVQEKFVCLNC | |
| | GFETHADEQAALNIARSWLFLRSQEYKKYQTNKTTGNTDKRAFVETWQSFYRKKLKEV | |
| | WKPAVGSPAAKRVKLD | |
| CasX 812 | MAPAAKRVKLDSRQEIKRINKIRRRLVKDSNTKKAGKTGPMKTLLVRVMTPDLRERLE | 19758 |
| with | NLRKKPENIPQPISNTSRANLNKLLTDYTEMKKAILHVYWEEFQKDPVGLMSRVAQPA | |
| flanking | SKKIDQNKLKPEMDEKGNLTTAGFACSQCGQPLFVYKLEQVSEKGKAYTNYFGRCNVA | |
| c-MYC | EHEKLILLAQLKPEKDSDEAVTYSLGKFGQRALDFYSIHVTKESTHPVKPLAQIAGNR | |
| NLSes | YASGPVGKALSDACMGTIASFLSKYQDIIIEHQKVVKGNQKRLESLRELAGKENLEYP | |
| | SVTLPPQPHTKEGVDAYNEVIARVRMWVNLNLWQKLKLSRDDAKPLLRLKKFPSFPLV | |
| | ERQANEVDWWDMVCNVKKLINEKKEDGKVFWQNLAGYKRQEALRPYLSSEEDRKKGKK | |
| | FARYQLGDLLLHLEKKHGEDWGKVYDEAWERIDKKVEGLSKHIKLEEERRSEDAQSKA | |
| | ALTDWLRAKASFVIEGLKEADKDEFCRCELKLQKWYGDLRGKPFAIEAENSILDISGF | |
| | SKQYNCAFIWQKDGVKKLNLYLIINYFKGGKLRFKKIKPEAFEANRFYTVINKKSGEI | |
| | VPMEVNFNFDDPNLIILPLAFGKRQGREFIWNDLLSLETGSLKLANGRVIEKTLYNRR | |
| | TRQDEPALFVALTFERREVLDSSNIKPMNLIGVDRGENIPAVIALTDPEGCPLSRFKD | |
| | SLGNPTHILRIGESYKEKQRTIQAKKEVEQRRAGGYSRKYASKAKNLADDMVRNTARD | |
| | LLYYAVTQDAMLIFENLSRGFGRQGKRTFMAERQYTRMEDWLTAKLAYEGLPSKTYLS | |
| | KTLAQYTSKTCSNCGFTITSADYDRVLEKLKKTATGWMTTINGKELKVEGQITYYNRY | |
| | KRQNVVKDLSVELDRLSEESVNNDISSWTKGRSGEALSLLKKRFSHRPVQEKFVCLNC | |
| | GFETHADEQAALNIARSWLFLRSQEYKKYQTNKTTGNTDKRAFVETWQSFYRKKLKEV | |
| | WKPAVGSPAAKRVKLD | |

Synthesis of gRNAs:

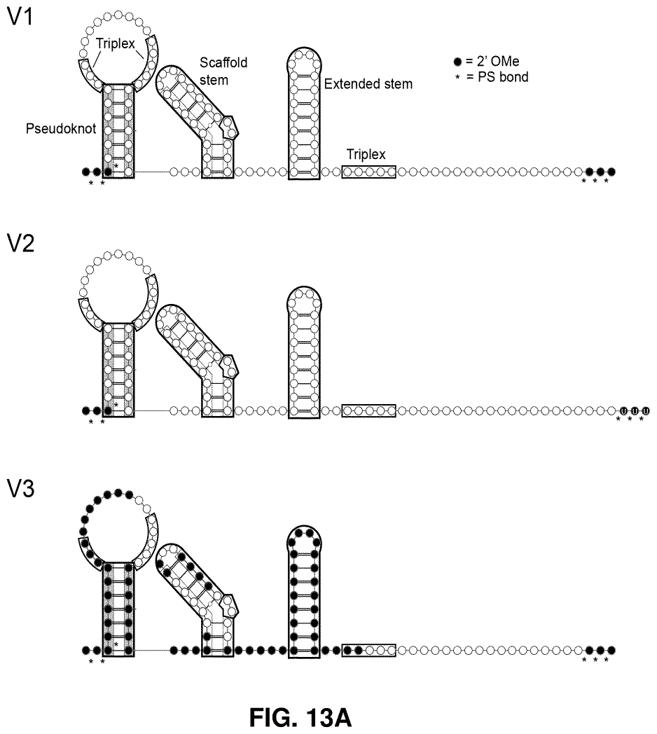

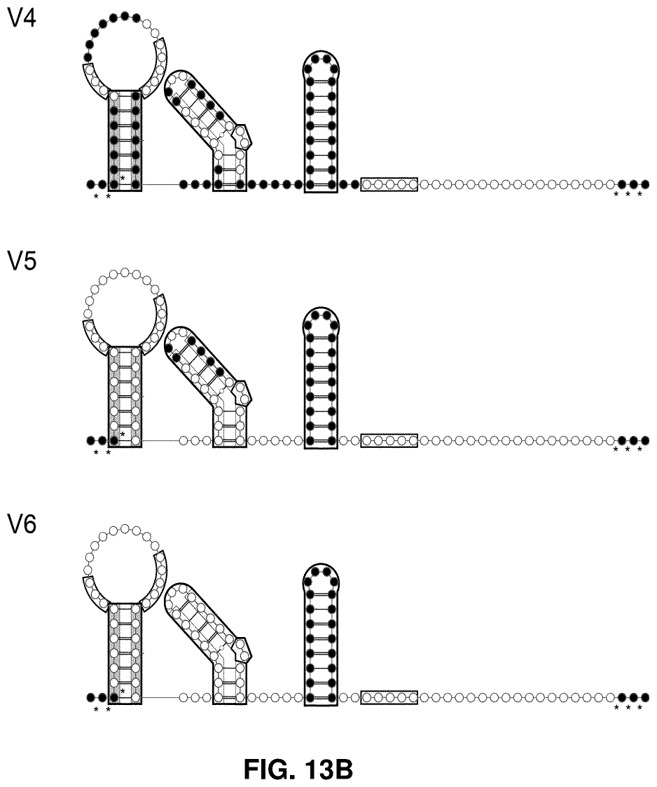

In this example, gRNAs targeting the human PCSK9 locus were designed using gRNA scaffold 316 and chemically synthesized. The sequences of the PCSK9-targeting gRNAs with the v1 modification profile (as described in Example 7, below) are listed in Table 26. Schematics of the sites of chemical modifications for the v1-v6 profiles of the gRNA scaffold variant 316 are shown in FIG. 13A and FIG. 13B.

TABLE 26
Sequences of chemically modified gRNAs targeting the human PCSK9 locus
assayed in this example

| gRNA ID (scaffold | | | SEQ ID |
|---|---|---|---|
| variant-spacer) | Target | gRNA sequence | NO |
| 316-6.1 (v1) | human | mA*mC*mU*GGCGCUUCUAUCUGAUUACUCUGAGCGCCAU | 19759 |
| | PCSK9 | CACCAGCGACUAUGUCGUAGUGGGUAAAGCUCCCUCUUCG | |
| | | GAGGGAGCAUCAGAGGAGGAGGACGGCCUGGC*mC*mG*m | |
| | | A | |
| 316-6.8 (v1) | human | mA*mC*mU*GGCGCUUCUAUCUGAUUACUCUGAGCGCCAU | 19760 |
| | PCSK9 | CACCAGCGACUAUGUCGUAGUGGGUAAAGCUCCCUCUUCG | |
| | | GAGGGAGCAUCAGAGUGGCUUCCUGGUGAAGA*mU*mG*m | |
| | | A | |

### Formulation of Lipid Nanoparticles (LNPs):

CasX mRNA and gRNA were encapsulated into LNPs using GenVoy-ILM™ lipids on the Precision NanoSystems Inc. (PNI) Ignite™ Benchtop System and following the manufacturer's guidelines. GenVoy-ILM™ lipids are manufactured by PNI, with a proprietary composition of ionizable lipid: DSPC: cholesterol: stabilizer at 50:10:37.5:2.5 mol %.

Briefly, to formulate LNPs, equal mass ratios of CasX mRNA and gRNA were diluted in PNI Formulation Buffer, pH 4.0. GenVoy-ILM™ was diluted 1:1 in anhydrous ethanol. mRNA/gRNA co-formulations were performed using a 6:1 N/P ratio. The RNA and lipids were run through a PNI laminar flow cartridge at a predetermined flow rate ratio (RNA: Genvoy-ILM™) on the PNI Ignite™ Benchtop System. After formulation, the LNPs were diluted in PBS, pH 7.4, to decrease the ethanol concentration and increase the pH, which increases the stability of the particles. Buffer exchange of the mRNA/sgRNA-LNPs was achieved by overnight dialysis into PBS, pH 7.4, at 4° C. using 10k Slide-A-Lyzer™ Dialysis Cassettes (Thermo Scientific™). Following dialysis, the mRNA/gRNA-LNPs were concentrated to >0.5 mg/mL using 100 kDa Amicon®-Ultra Centrifugal Filters (Millipore) and then filter-sterilized. Formulated LNPs were analyzed on a Stunner (Unchained Labs) to determine their diameter and polydispersity index (PDI). Encapsulation efficiency and RNA concentration was determined by RiboGreen™ assay using Invitrogen's Quant-iT™ Ribogreen™ RNA assay kit. LNPs were used in various experiments as described herein to deliver CasX mRNA and gRNA to target cells and tissue.

Delivery of LNPs Encapsulating CasX mRNA and Targeting gRNA into Primary Human Hepatocytes:

Two lots (lot #31 and lot #51) of primary human hepatocytes derived from two different donors (Lonza Biologics), were used in these experiments to assess CasX:gRNA-mediated editing at the human PCSK9 locus when delivered by LNPs. For each lot, ˜50,000 cells, cultured in Williams' E media supplemented with FBS, PenStrep, L-glutamine, ITS (insulin, transferrin, sodium selenite), dexamethasone, and Z-VAD-FMK, were seeded per well in a 96-well plate. The next day, seeded cells were treated with varying concentrations of LNPs, which were prepared in five 3-fold serial dilutions starting at 1,200 ng. These LNPs were formulated to encapsulate CasX 515 or CasX 812 mRNA and a PCSK9-targeting gRNA incorporating scaffold variant 316 with either spacer 6.1 or 6.8 (v1; see Table 25). Media was changed two days after LNP treatment, and cells were cultured for three additional days prior to harvesting 1) the media supernatant to measure PCSK9 secretion levels and 2) treated cells for gDNA extraction for editing assessment at the PCSK9 locus by NGS. Briefly, for editing assessment, amplicons were amplified from 200 ng of extracted gDNA with primers targeting the human PCSK9 locus and processed as described in Example 5. PCSK9 secretion levels were measured by ELISA using the BioLegendR ELISA MAX™ kit following the manufacturer's instructions. Treatment with LNPs co-encapsulating a non-targeting gRNA with CasX 515 mRNA served as an experimental control.

### Results

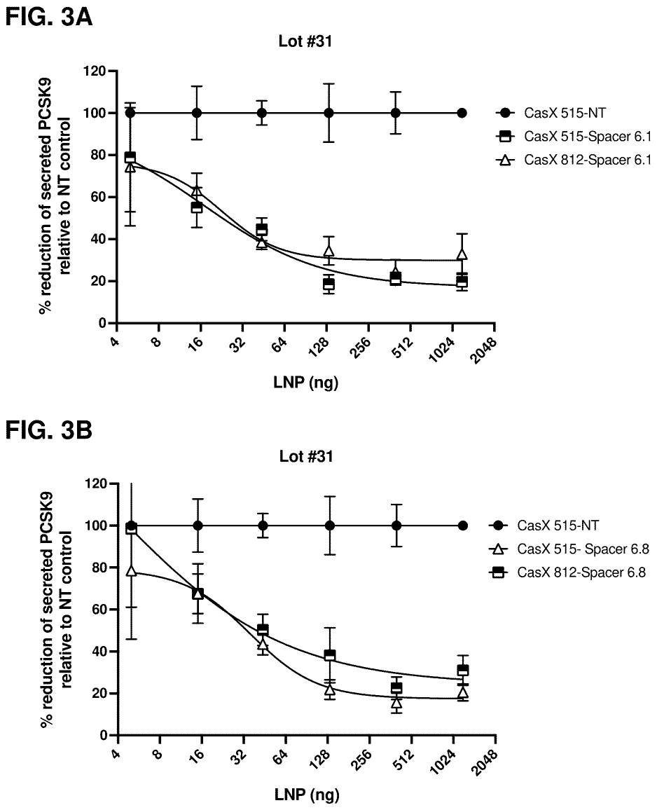

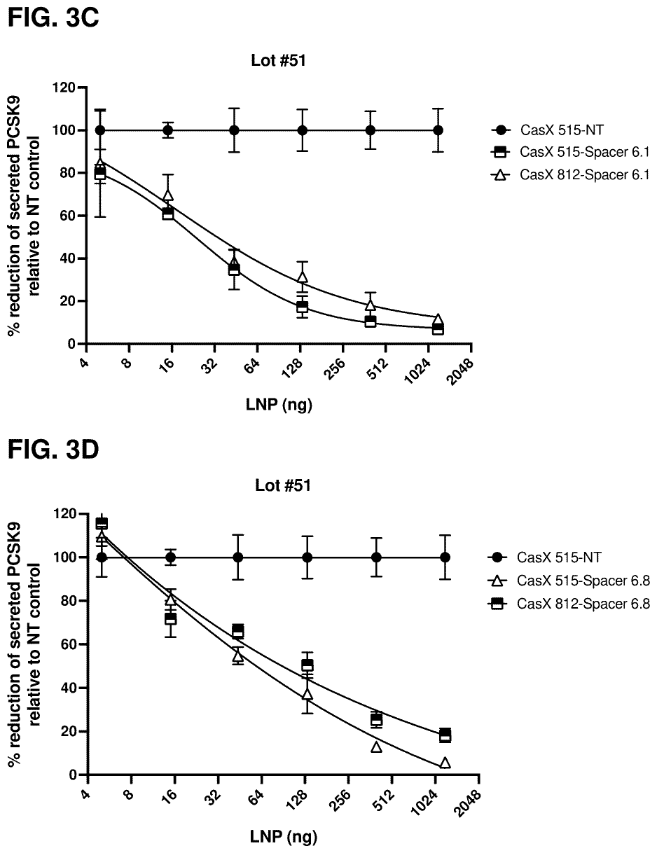

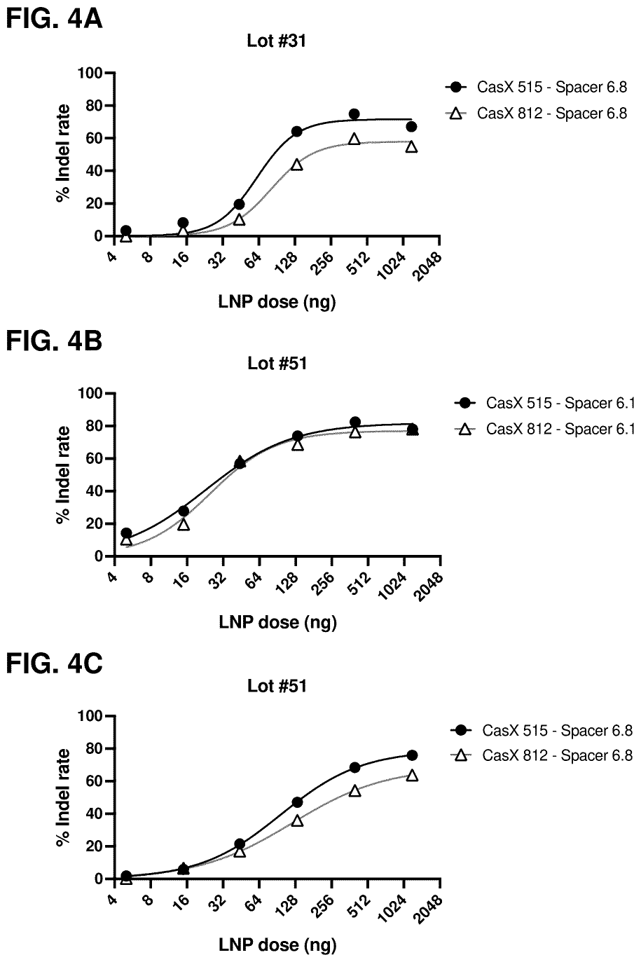

Two lots of primary human hepatocytes were treated with LNPs, which co-encapsulated either CasX 515 or CasX 812 mRNA and a PCSK9-targeting gRNA, at various doses and harvested five days post-treatment to assess effects on PCSK9 secretion (FIGS. 3A-3D) and editing at the PCSK9 locus (FIGS. 4A-4C). The results in FIGS. 3A-3D demonstrate that the effects from treatment with LNPs to deliver either CasX 515 or CasX 812 mRNA were comparable, such that similar levels of reduced PCSK9 secretion were observed in a dose-dependent manner. Furthermore, the data in FIGS. 4A-4C show that use of either CasX 515 or CasX 812 mRNA resulted in similar levels of editing at the PCSK9 locus in primary human hepatocytes in a dose-dependent manner, corroborating findings observed in FIGS. 3A-3D.

Altogether, the results from these experiments demonstrate that delivery of LNPs encapsulating an mRNA encoding CasX and a chemically-modified PCSK9-targeting gRNA was able to induce efficacious editing at the endogenous human PCSK9 locus in primary human hepatocytes, which resulted in substantial reduction in secreted PCSK9 levels.

### Example 7: Design and Assessment of Modified gRNAs in Improving Editing when Delivered Together with CasX mRNA In Vitro and In Vivo

Experiments were performed to identify new gRNA variant sequences and demonstrate that chemical modifications of these gRNA variants enhance the editing efficiency of the CasX:gRNA system when delivered in vitro in conjunction with CasX mRNA.

### Materials and Methods

Synthesis of gRNAs:

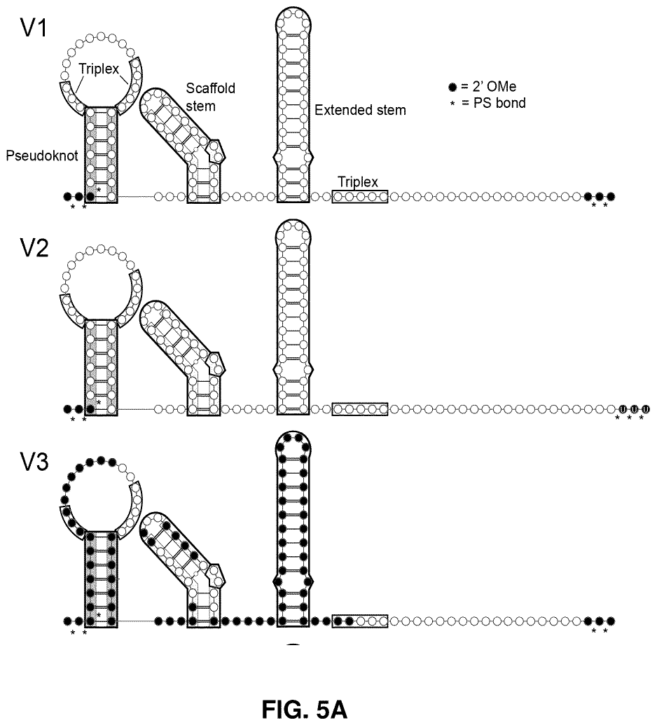

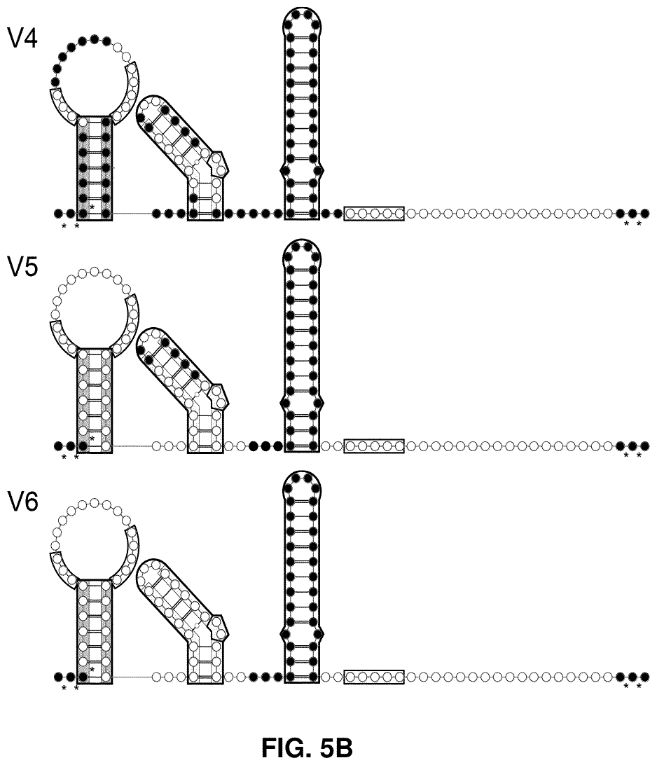

All gRNAs tested in this example were chemically synthesized and were derived from gRNA scaffolds 174, 235, and 316. The sequences of gRNA scaffolds 174, 235, and 316 and their chemical modification profiles are listed in Table 27. The sequences of the resulting gRNAs, including spacers targeting PCSK9, B2M, or ROSA26, and their chemical modification profiles assayed in this example are listed in Table 28. A schematic of the structure of gRNA scaffold variants 174, 235, and 316 are shown in FIGS. 8A-8C, respectively, and the sites of chemical modifications of the gRNA variants are shown schematically in FIGS. 5A, 5B, 7, 13A, and 13B.

TABLE 27
Sequences of gRNA scaffolds with their different chemical modification
profiles (denoted by version number), where “NNNNNNNNNNNNNNNNNNNN” is a
spacer placeholder. Chemical modifications: * = phosphorothioate bond; m = 2′OMe
modification

| gRNA | | SEQ ID |
|---|---|---|
| scaffold | | NO |
| (version) | gRNA sequence | |
| 174 (v0) | ACUGGCGCUUUUAUCUGAUUACUUUGAGAGCCAUCACCAGCGACUAUGUCGUAGUGG | 19703 |
| | GUAAAGCUCCCUCUUCGGAGGGAGCAUCAAAGNNNNNNNNNNNNNNNNNNNN | |
| 174 (v1) | mA*mC*mU*GGCGCUUUUAUCUGAUUACUUUGAGAGCCAUCACCAGCGACUAUGUCG | 19704 |
| | UAGUGGGUAAAGCUCCCUCUUCGGAGGGAGCAUCAAAGNNNNNNNNNNNNNNNNNmN | |
| | *mN*mN | |
| 174 (v2) | mA*mC*mU*GGCGCUUUUAUCUGAUUACUUUGAGAGCCAUCACCAGCGACUAUGUCG | 19705 |
| | UAGUGGGUAAAGCUCCCUCUUCGGAGGGAGCAUCAAAGNNNNNNNNNNNNNNNNNNN | |
| | N*mU*mU*mU | |
| 174 (v3) | mA*mC*mU*mGmGmCmGmCmUmUmUmUmAmUmCmUmGmAmUUACUUUGmAmGmAmGm | 19706 |
| | CmCmAmUmCmAmCmCAGCGAmCmUAUmGmUmCmGUAGUGmGmGmUmAmAmAmGmCmU | |
| | mCmCmCmUmCmUmUmCmGmGmAmGmGmGmAmGmCmAmUmCmAAAGNNNNNNNNNNNN | |
| | NNNNNmN*mN*mN | |
| 174 (v4) | mA*mC*mU*mGmGmCmGmCUUUUmAmUmCmUmGmAmUUACUUUGmAmGmAmGmCmCm | 19707 |
| | AmUmCmAmCmCAGCGAmCmUAUmGmUmCmGUAGUGmGmGmUmAmAmAmGmCmUmCmC | |
| | mCmUmCmUmUmCmGmGmAmGmGmGmAmGmCmAmUCAAAGNNNNNNNNNNNNNNNNNm | |
| | N*mN*mN | |
| 174 (v5) | mA*mC*mU*GGCGCUUUUAUCUGAUUACUUUGAGAGCCAUCACCAGCGAmCmUAUmG | 19708 |
| | mUmCmGUAGUGGGUAAAmGmCmUmCmCmCmUmCmUmUmCmGmGmAmGmGmGmAmGmC | |
| | AUCAAAGNNNNNNNNNNNNNNNNNmN*mN*mN | |
| 174 (v6) | mA*mC*mU*GGCGCUUUUAUCUGAUUACUUUGAGAGCCAUCACCAGCGACUAUGUCG | 19709 |
| | UAGUGGGUAAAmGmCmUmCmCmCmUmCmUmUmCmGmGmAmGmGmGmAmGmCAUCAAA | |
| | GNNNNNNNNNNNNNNNNNmN*mN*mN | |
| 174 (v7) | mA*mC*mU*GGmCGmCmUUUUAmUmCUGAUUACUUUGmAmGAGCCmAmUmCmAmCCA | 19710 |
| | GCmGmAmCmUAUmGmUmCmGUAGUGGmGmUAmAmAmGmCmUmCmCmCmUmCmUmUmC | |
| | mGmGmAmGmGmGmAmGmCmAmUCAAAGNNNNNNNNNNNNNNNNN*mN*mN*mN | |
| 174 (v8) | mA*mC*mU*GGCGCUUUUAUCUGAUUACUUUGAGAGCCmAmUmCmAmCCAGCmGmAm | 19711 |
| | CmUAUmGmUmCmGUAGUGGmGmUmAmAAmGmCmUmCmCmCmUmCmUmUmCmGmGmAm | |
| | GmGmGmAmGmCmAmUCAAAGNNNNNNNNNNNNNNNNN*mN*mN*mN | |
| 174 (v9) | mA*mC*mU*GGmCmGCmUUUUAmUmCUGAUUACUUUGmAmGAGCCAUCACCAGCmGm | 19712 |
| | AmCmUAUmGmUmCmGUAGUGGGUAAAmGmCmUmCmCmCmUmCmUmUmCmGmGmAmGm | |
| | GmGmAmGmCAUCAAAGNNNNNNNNNNNNNNNNN*mN*mN*mN | |
| 235 (v0) | ACUGGCGCUUCUAUCUGAUUACUCUGAGCGCCAUCACCAGCGACUAUGUCGUAGUGG | 19713 |
| | GUAAAGCCGCUUACGGACUUCGGUCCGUAAGAGGCAUCAGAGNNNNNNNNNNNNNNN | |
| | NNNNN | |
| 235 (v1) | mA*mC*mU*GGCGCUUCUAUCUGAUUACUCUGAGCGCCAUCACCAGCGACUAUGUCG | 19714 |
| | UAGUGGGUAAAGCCGCUUACGGACUUCGGUCCGUAAGAGGCAUCAGAGNNNNNNNNN | |
| | NNNNNNNNmN*mN*mN | |
| 235 (v2) | mA*mC*mU*GGCGCUUCUAUCUGAUUACUCUGAGCGCCAUCACCAGCGACUAUGUCG | 19715 |
| | UAGUGGGUAAAGCCGCUUACGGACUUCGGUCCGUAAGAGGCAUCAGAGNNNNNNNNN | |
| | NNNNNNNNNNN*mU*mU*mU | |
| 235 (v3) | mA*mC*mU*mGmGmCmGmCmUmUmCmUmAmUmCmUmGmAmUUACUCUGmAmGmCmGm | 19716 |
| | CmCmAmUmCmAmCmCAGCGAmCmUAUmGmUmCmGUAGUGmGmGmUmAmAmAmGmCmC | |
| | mGmCmUmUmAmCmGmGmAmCmUmUmCmGmGmUmCmCmGmUmAmAmGmAmGmGmCmAm | |
| | UmCmAGAGNNNNNNNNNNNNNNNNNmN*mN*mN | |
| 235 (v4) | mA*mC*mU*mGmGmCmGmCUUCUmAmUmCmUmGmAmUUACUCUGmAmGmCmGmCmCm | 19717 |
| | AmUmCmAmCmCAGCGAmCmUAUmGmUmCmGUAGUGmGmGmUmAmAmAmGmCmCmGmC | |
| | mUmUmAmCmGmGmAmCmUmUmCmGmGmUmCmCmGmUmAmAmGmAmGmGmCmAmUCAG | |
| | AGNNNNNNNNNNNNNNNNNmN*mN*mN | |
| 235 (v5) | mA*mC*mU*GGCGCUUCUAUCUGAUUACUCUGAGCGCCAUCACCAGCGAmCmUAUmG | 19718 |
| | mUmCmGUAGUGGGUmAmAmAmGmCmCmGmCmUmUmAmCmGmGmAmCmUmUmCmGmGm | |
| | UmCmCmGmUmAmAmGmAmGmGmCAUCAGAGNNNNNNNNNNNNNNNNNmN*mN*mN | |
| 235 (v6) | mA*mC*mU*GGCGCUUCUAUCUGAUUACUCUGAGCGCCAUCACCAGCGACUAUGUCG | 19719 |
| | UAGUGGGUmAmAmAmGmCmCmGmCmUmUmAmCmGmGmAmCmUmUmCmGmGmUmCmCm | |
| | GmUmAmAmGmAmGmGmCAUCAGAGNNNNNNNNNNNNNNNNNmN*mN*mN | |
| 235 (v7) | mA*mC*mU*GGmCGmCmUUCUAmUmCUGAUUACUCUGmAmGCGCCmAmUmCmAmCCA | 19720 |
| | GCmGmAmCmUAUmGmUmCmGUAGUGGmGmUmAmAAmGmCmCmGmCmUmUmAmCmGmG | |
| | mAmCmUmUmCmGmGmUmCmCmGmUmAmAmGmAmGmGmCmAmUCAGAGNNNNNNNNNN | |
| | NNNNNNN*mN*mN*mN | |
| 235 (v8) | mA*mC*mU*GGCGCUUCUAUCUGAUUACUCUGAGCGCCmAmUmCmAmCCAGCmGmAm | 19721 |
| | CmUAUmGmUmCmGUAGUGGmGmUmAmAAmGmCmCmGmCmUmUmAmCmGmGmAmCmUm | |
| | UmCmGmGmUmCmCmGmUmAmAmGmAmGmGmCmAmUCAGAGNNNNNNNNNNNNNNNNN | |
| | *mN*mN*mN | |
| 235 (v9) | mA*mC*mU*GGmCGmCmUUCUAmUmCUGAUUACUCUGmAmGCGCCAUCACCAGCmGm | 19722 |
| | AmCmUAUmGmUmCmGUAGUGGGUAAAmGmCmCmGmCmUmUmAmCmGmGmAmCmUmUm | |
| | CmGmGmUmCmCmGmUmAmAmGmAmGmGmCAUCAGAGNNNNNNNNNNNNNNNNN*mN* | |
| | mN*mN | |
| 316 (v0) | ACUGGCGCUUCUAUCUGAUUACUCUGAGCGCCAUCACCAGCGACUAUGUCGUAGUGG | 19723 |
| | GUAAAGCUCCCUCUUCGGAGGGAGCAUCAGAGNNNNNNNNNNNNNNNNNNNN | |
| 316 (v1) | mA*mC*mU*GGCGCUUCUAUCUGAUUACUCUGAGCGCCAUCACCAGCGACUAUGUCG | 19724 |
| | UAGUGGGUAAAGCUCCCUCUUCGGAGGGAGCAUCAGAGNNNNNNNNNNNNNNNNN*m | |
| | N*mN*mN | |
| 316 (v2) | mA*mC*mU*GGCGCUUCUAUCUGAUUACUCUGAGCGCCAUCACCAGCGACUAUGUCG | 19725 |
| | UAGUGGGUAAAGCUCCCUCUUCGGAGGGAGCAUCAGAGNNNNNNNNNNNNNNNNNNN | |
| | N*mU*mU*mU | |
| 316 (v3) | mA*mC*mU*mGmGmCmGmCmUmUmCmUmAmUmCmUmGmAmUUACUCUGmAmGmCmGm | 19726 |
| | CmCmAmUmCmAmCmCAGCGAmCmUAUmGmUmCmGUAGUGmGmGmUmAmAmAmGmCmU | |
| | mCmCmCmUmCmUmUmCmGmGmAmGmGmGmAmGmCmAmUmCmAGAGNNNNNNNNNNNN | |
| | NNNNN*mN*mN*mN | |
| 316 (v4) | mA*mC*mU*mGmGmCmGmCUUCUmAmUmCmUmGmAmUUACUCUGmAmGmCmGmCmCm | 19727 |
| | AmUmCmAmCmCAGCGAmCmUAUmGmUmCmGUAGUGmGmGmUmAmAmAmGmCmUmCmC | |
| | mCmUmCmUmUmCmGmGmAmGmGmGmAmGmCmAmUCAGAGNNNNNNNNNNNNNNNNN* | |
| | mN*mN*mN | |
| 316 (v5) | mA*mC*mU*GGCGCUUCUAUCUGAUUACUCUGAGCGCCAUCACCAGCGAmCmUAUmG | 19728 |
| | mUmCmGUAGUGGGUAAAmGmCmUmCmCmCmUmCmUmUmCmGmGmAmGmGmGmAmGmC | |
| | AUCAGAGNNNNNNNNNNNNNNNNN*mN*mN*mN | |
| 316 (v6) | mA*mC*mU*GGCGCUUCUAUCUGAUUACUCUGAGCGCCAUCACCAGCGACUAUGUCG | 19729 |
| | UAGUGGGUAAAmGmCmUmCmCmCmUmCmUmUmCmGmGmAmGmGmGmAmGmCAUCAGA | |
| | GNNNNNNNNNNNNNNNNN*mN*mN*mN | |
| 316 (v7) | mA*mC*mU*GGmCGmCmUUCUAmUmCUGAUUACUCUGmAmGCGCCmAmUmCmAmCCA | 19730 |
| | GCmGmAmCmUAUmGmUmCmGUAGUGGmGmUmAmAAmGmCmUmCmCmCmUmCmUmUmC | |
| | mGmGmAmGmGmGmAmGmCmAmUCAGAGNNNNNNNNNNNNNNNNN*mN*mN*mN | |
| 316 (v8) | mA*mC*mU*GGCGCUUCUAUCUGAUUACUCUGAGCGCCmAmUmCmAmCCAGCmGmAm | 19731 |
| | CmUAUmGmUmCmGUAGUGGmGmUmAmAAmGmCmUmCmCmCmUmCmUmUmCmGmGmAm | |
| | GmGmGmAmGmCmAmUCAGAGNNNNNNNNNNNNNNNNN*mN*mN*mN | |
| 316 (v9) | mA*mC*mU*GGmCGmCmUUCUAmUmCUGAUUACUCUGmAmGCGCCAUCACCAGCmGm | 19732 |
| | AmCmUAUmGmUmCmGUAGUGGGUAAAmGmCmUmCmCmCmUmCmUmUmCmGmGmAmGm | |
| | GmGmAmGmCAUCAGAGNNNNNNNNNNNNNNNNN*mN*mN*mN | |

TABLE 28
Sequences of gRNAs with their different chemical modification profiles
(denoted by version number) assayed in this example. Chemical modifications: * =
phosphorothioate bond; m = 2′OMe modification

| gRNA ID | | | |
|---|---|---|---|
| (scaffold | | | SEQ |
| variant-spacer) | Target | gRNA sequence | ID NO |
| 174-6.7 | human | ACUGGCGCUUUUAUCUGAUUACUUUGAGAGCCAUCACCAGCGAC | 19761 |
| (v0) | PCSK9 | UAUGUCGUAGUGGGUAAAGCUCCCUCUUCGGAGGGAGCAUCAAA | |
| | | GUCCUGGCUUCCUGGUGAAGA | |
| 174-6.7 | human | mA*mC*mU*GGCGCUUUUAUCUGAUUACUUUGAGAGCCAUCACC | 19762 |
| (v1) | PCSK9 | AGCGACUAUGUCGUAGUGGGUAAAGCUCCCUCUUCGGAGGGAGC | |
| | | AUCAAAGUCCUGGCUUCCUGGUGAmA*mG*mA | |
| 174-6.8 | human | ACUGGCGCUUUUAUCUGAUUACUUUGAGAGCCAUCACCAGCGAC | 19763 |
| (v0) | PCSK9 | UAUGUCGUAGUGGGUAAAGCUCCCUCUUCGGAGGGAGCAUCAAA | |
| | | GUGGCUUCCUGGUGAAGAUGA | |
| 174-6.8 | human | mA*mC*mU*GGCGCUUUUAUCUGAUUACUUUGAGAGCCAUCACC | 19764 |
| (v1) | PCSK9 | AGCGACUAUGUCGUAGUGGGUAAAGCUCCCUCUUCGGAGGGAGC | |
| | | AUCAAAGUGGCUUCCUGGUGAAGAmU*mG*mA | |
| 174-7.9 | human | ACUGGCGCUUUUAUCUGAUUACUUUGAGAGCCAUCACCAGCGAC | 19765 |
| (v0) | B2M | UAUGUCGUAGUGGGUAAAGCUCCCUCUUCGGAGGGAGCAUCAAA | |
| | | GGUGUAGUACAAGAGAUAGAA | |
| 174-7.9 | human | mA*mC*mU*GGCGCUUUUAUCUGAUUACUUUGAGAGCCAUCACC | 19766 |
| (v1) | B2M | AGCGACUAUGUCGUAGUGGGUAAAGCUCCCUCUUCGGAGGGAGC | |
| | | AUCAAAGGUGUAGUACAAGAGAUAmG*mA*mA | |
| 316-6.7 | human | ACUGGCGCUUCUAUCUGAUUACUCUGAGCGCCAUCACCAGCGAC | 19767 |
| (v0) | PCSK9 | UAUGUCGUAGUGGGUAAAGCUCCCUCUUCGGAGGGAGCAUCAGA | |
| | | GUCCUGGCUUCCUGGUGAAGA | |
| 316-6.7 | human | mA*mC*mU*GGCGCUUCUAUCUGAUUACUCUGAGCGCCAUCACC | 19768 |
| (v1′) | PCSK9 | AGCGACUAUGUCGUAGUGGGUAAAGCUCCCUCUUCGGAGGGAGC | |
| | | AUCAGAGUCCUGGCUUCCUGGUGAmA*mG*mA | |
| 316-6.8 | human | ACUGGCGCUUCUAUCUGAUUACUCUGAGCGCCAUCACCAGCGAC | 19769 |
| (v0) | PCSK9 | UAUGUCGUAGUGGGUAAAGCUCCCUCUUCGGAGGGAGCAUCAGA | |
| | | GUGGCUUCCUGGUGAAGAUGA | |
| 316-6.8 | human | mA*mC*mU*GGCGCUUCUAUCUGAUUACUCUGAGCGCCAUCACC | 19770 |
| (v1′) | PCSK9 | AGCGACUAUGUCGUAGUGGGUAAAGCUCCCUCUUCGGAGGGAGC | |
| | | AUCAGAGUGGCUUCCUGGUGAAGAmU*mG*mA | |
| 316-7.9 | human | ACUGGCGCUUCUAUCUGAUUACUCUGAGCGCCAUCACCAGCGAC | 19771 |
| (v0) | B2M | UAUGUCGUAGUGGGUAAAGCUCCCUCUUCGGAGGGAGCAUCAGA | |
| | | GGUGUAGUACAAGAGAUAGAA | |
| 316-7.9 | human | mA*mC*mU*GGCGCUUCUAUCUGAUUACUCUGAGCGCCAUCACC | 19772 |
| (v1′) | B2M | AGCGACUAUGUCGUAGUGGGUAAAGCUCCCUCUUCGGAGGGAGC | |
| | | AUCAGAGGUGUAGUACAAGAGAUAmG*mA*mA | |
| 174-7.37 | human | ACUGGCGCUUUUAUCUgAUUACUUUGAGAGCCAUCACCAGCGAC | 19773 |
| (v0) | B2M | UAUGUCGUAgUGGGUAAAGCUCCCUCUUCGGAGGGAGCAUCAAA | |
| | | GGGCCGAGAUGUCUCGCUC | |
| 174-7.37 | human | mA*mC*mU*GGCGCUUUUAUCUgAUUACUUUGAGAGCCAUCACC | 19774 |
| (v1*) | B2M | AGCGACUAUGUCGUAgUGGGUAAAGCUCCCUCUUCGGAGGGAGC | |
| | | AUCAAAGGGCCGAGAUGUCUCG*mC*mU*mC | |
| 235-6.7 | human | ACUGGCGCUUCUAUCUGAUUACUCUGAGCGCCAUCACCAGCGAC | 19775 |
| (v0) | PCSK9 | UAUGUCGUAGUGGGUAAAGCCGCUUACGGACUUCGGUCCGUAAG | |
| | | AGGCAUCAGAGUCCUGGCUUCCUGGUGAAGA | |
| 235-6.7 | human | mA*mC*mU*GGCGCUUCUAUCUGAUUACUCUGAGCGCCAUCACC | 19776 |
| (v1) | PCSK9 | AGCGACUAUGUCGUAGUGGGUAAAGCCGCUUACGGACUUCGGUC | |
| | | CGUAAGAGGCAUCAGAGUCCUGGCUUCCUGGUGAmA*mG*mA | |
| 235-6.7 | human | mA*mC*mU*GGCGCUUCUAUCUGAUUACUCUGAGCGCCAUCACC | 19777 |
| (v2) | PCSK9 | AGCGACUAUGUCGUAGUGGGUAAAGCCGCUUACGGACUUCGGUC | |
| | | CGUAAGAGGCAUCAGAGUCCUGGCUUCCUGGUGAAGAU*mU*mU | |
| | | *mU | |
| 235-6.7 | human | mA*mC*mU*mGmGmCmGmCmUmUmCmUmAmUmCmUmGmAmUUAC | 19778 |
| (v3) | PCSK9 | UCUGmAmGmCmGmCmCmAmUmCmAmCmCAGCGAmCmUAUmGmUm | |
| | | CmGUAGUGmGmGmUmAmAmAmGmCmCmGmCmUmUmAmCmGmGmA | |
| | | mCmUmUmCmGmGmUmCmCmGmUmAmAmGmAmGmGmCmAmUmCmA | |
| | | GAGUCCUGGCUUCCUGGUGAmA*mG*mA | |
| 235-6.7 | human | mA*mC*mU*mGmGmCmGmCUUCUmAmUmCmUmGmAmUUACUCUG | 19779 |
| (v4) | PCSK9 | mAmGmCmGmCmCmAmUmCmAmCmCAGCGAmCmUAUmGmUmCmGU | |
| | | AGUGmGmGmUmAmAmAmGmCmCmGmCmUmUmAmCmGmGmAmCmU | |
| | | mUmCmGmGmUmCmCmGmUmAmAmGmAmGmGmCmAmUCAGAGUCC | |
| | | UGGCUUCCUGGUGAmA*mG*mA | |
| 235-6.7 | human | mA*mC*mU*GGCGCUUCUAUCUGAUUACUCUGAGCGCCAUCACC | 19780 |
| (v5) | PCSK9 | AGCGAmCmUAUmGmUmCmGUAGUGGGUmAmAmAmGmCmCmGmCm | |
| | | UmUmAmCmGmGmAmCmUmUmCmGmGmUmCmCmGmUmAmAmGmAm | |
| | | GmGmCAUCAGAGUCCUGGCUUCCUGGUGAmA*mG*mA | |
| 235-6.7 | human | mA*mC*mU*GGCGCUUCUAUCUGAUUACUCUGAGCGCCAUCACC | 19781 |
| (v6) | PCSK9 | AGCGACUAUGUCGUAGUGGGUmAmAmAmGmCmCmGmCmUmUmAm | |
| | | CmGmGmAmCmUmUmCmGmGmUmCmCmGmUmAmAmGmAmGmGmCA | |
| | | UCAGAGUCCUGGCUUCCUGGUGAmA*mG*mA | |
| 235-6.8 | human | ACUGGCGCUUCUAUCUGAUUACUCUGAGCGCCAUCACCAGCGAC | 19782 |
| (v0) | PCSK9 | UAUGUCGUAGUGGGUAAAGCCGCUUACGGACUUCGGUCCGUAAG | |
| | | AGGCAUCAGAGUGGCUUCCUGGUGAAGAUGA | |
| 235-6.8 | human | mA*mC*mU*GGCGCUUCUAUCUGAUUACUCUGAGCGCCAUCACC | 19783 |
| (v1) | PCSK9 | AGCGACUAUGUCGUAGUGGGUAAAGCCGCUUACGGACUUCGGUC | |
| | | CGUAAGAGGCAUCAGAGUGGCUUCCUGGUGAAGAmU*mG*mA | |
| 235-6.8 | human | mA*mC*mU*GGCGCUUCUAUCUGAUUACUCUGAGCGCCAUCACC | 19784 |
| (v2) | PCSK9 | AGCGACUAUGUCGUAGUGGGUAAAGCCGCUUACGGACUUCGGUC | |
| | | CGUAAGAGGCAUCAGAGUGGCUUCCUGGUGAAGAUGA*mU*mU* | |
| | | mU | |
| 235-6.8 | human | mA*mC*mU*mGmGmCmGmCmUmUmCmUmAmUmCmUmGmAmUUAC | 19785 |
| (v3) | PCSK9 | UCUGmAmGmCmGmCmCmAmUmCmAmCmCAGCGAmCmUAUmGmUm | |
| | | CmGUAGUGmGmGmUmAmAmAmGmCmCmGmCmUmUmAmCmGmGmA | |
| | | mCmUmUmCmGmGmUmCmCmGmUmAmAmGmAmGmGmCmAmUmCmA | |
| | | GAGUGGCUUCCUGGUGAAGAmU*mG*mA | |
| 235-6.8 | human | mA*mC*mU*mGmGmCmGmCUUCUmAmUmCmUmGmAmUUACUCUG | 19786 |
| (v4) | PCSK9 | mAmGmCmGmCmCmAmUmCmAmCmCAGCGAmCmUAUmGmUmCmGU | |
| | | AGUGmGmGmUmAmAmAmGmCmCmGmCmUmUmAmCmGmGmAmCmU | |
| | | mUmCmGmGmUmCmCmGmUmAmAmGmAmGmGmCmAmUCAGAGUGG | |
| | | CUUCCUGGUGAAGAmU*mG*mA | |
| 235-6.8 | human | mA*mC*mU*GGCGCUUCUAUCUGAUUACUCUGAGCGCCAUCACC | 19787 |
| (v5) | PCSK9 | AGCGAmCmUAUmGmUmCmGUAGUGGGUmAmAmAmGmCmCmGmCm | |
| | | UmUmAmCmGmGmAmCmUmUmCmGmGmUmCmCmGmUmAmAmGmAm | |
| | | GmGmCAUCAGAGUGGCUUCCUGGUGAAGAmU*G*mA | |
| 235-6.8 | human | mA*mC*mU*GGCGCUUCUAUCUGAUUACUCUGAGCGCCAUCACC | 19788 |
| (v6) | PCSK9 | AGCGACUAUGUCGUAGUGGGUmAmAmAmGmCmCmGmCmUmUmAm | |
| | | CmGmGmAmCmUmUmCmGmGmUmCmCmGmUmAmAmGmAmGmGmCA | |
| | | UCAGAGUGGCUUCCUGGUGAAGAmU*mG*mA | |
| 316-27.107 | mouse | ACUGGCGCUUCUAUCUGAUUACUCUGAGCGCCAUCACCAGCGAC | 19789 |
| (v0) | PCSK9 | UAUGUCGUAGUGGGUAAAGCUCCCUCUUCGGAGGGAGCAUCAGA | |
| | | GCUGGCUUCUUGGUGAAGAUG | |
| 316-27.107 | mouse | mA*mC*mU*GGCGCUUCUAUCUGAUUACUCUGAGCGCCAUCACC | 19790 |
| (v1) | PCSK9 | AGCGACUAUGUCGUAGUGGGUAAAGCUCCCUCUUCGGAGGGAGC | |
| | | AUCAGAGCUGGCUUCUUGGUGAAG*mA*mU*mG | |
| 316-27.107 | mouse | mA*mC*mU*GGmCGmCmUUCUAmUmCUGAUUACUCUGmAmGCGC | 19791 |
| (v7) | PCSK9 | CmAmUmCmAmCCAGCmGmAmCmUAUmGmUmCmGUAGUGGmGmUm | |
| | | AmAAmGmCmUmCmCmCmUmCmUmUmCmGmGmAmGmGmGmAmGmC | |
| | | mAmUCAGAGCUGGCUUCUUGGUGAAG*mA*mU*mG | |
| 316-27.107 | mouse | mA*mC*mU*GGCGCUUCUAUCUGAUUACUCUGAGCGCCmAmUmC | 19792 |
| (v8) | PCSK9 | mAmCCAGCmGmAmCmUAUmGmUmCmGUAGUGGmGmUmAmAAmGm | |
| | | CmUmCmCmCmUmCmUmUmCmGmGmAmGmGmGmAmGmCmAmUCAG | |
| | | AGCUGGCUUCUUGGUGAAG*mA*mU*mG | |
| 316-27.107 | mouse | mA*mC*mU*GGmCGmCmUUCUAmUmCUGAUUACUCUGmAmGCGC | 19793 |
| (v9*) | PCSK9 | CAUCACCAGCmGmAmCmUAUmGmUmCmGUAGUGGGUAAAmGmCm | |
| | | UmCmCmCmUmCmUmUmCmGmGmAmGmGmGmAmGmCAUCAGAGCU | |
| | | GGCUUCUUGGUGAA*mG*mA*mU*mG | |
| 174-35.2 (v0) | ROSA26 | ACUGGCGCUUUUAUCUGAUUACUUUGAGAGCCAUCACCAGCGAC | 19794 |
| | | UAUGUCGUAGUGGGUAAAGCUCCCUCUUCGGAGGGAGCAUCAAA | |
| | | GAGAAGAUGGGCGGGAGUCUU | |
| 174-35.2 (v2) | ROSA26 | mA*mC*mU*GGCGCUUUUAUCUGAUUACUUUGAGAGCCAUCACC | 19795 |
| | | AGCGACUAUGUCGUAGUGGGUAAAGCUCCCUCUUCGGAGGGAGC | |
| | | AUCAAAGAGAAGAUGGGGGGGAGUCUU*mU*mU*mU | |
| 316-35.2 (v0) | ROSA26 | ACUGGCGCUUCUAUCUGAUUACUCUGAGCGCCAUCACCAGCGAC | 19796 |
| | | UAUGUCGUAGUGGGUAAAGCUCCCUCUUCGGAGGGAGCAUCAGA | |
| | | GAGAAGAUGGGCGGGAGUCUU | |
| 316-35.2 (v1) | ROSA26 | mA*mC*mU*GGCGCUUCUAUCUGAUUACUCUGAGCGCCAUCACC | 19797 |
| | | AGCGACUAUGUCGUAGUGGGUAAAGCUCCCUCUUCGGAGGGAGC | |
| | | AUCAGAGAGAAGAUGGGGGGAGU*mC*mU*mU | |
| 316-35.2 (v5) | ROSA26 | mA*mC*mU*GGCGCUUCUAUCUGAUUACUCUGAGCGCCAUCACC | 19798 |
| | | AGCGAmCmUAUmGmUmCmGUAGUGGGUAAAmGmCmUmCmCmCmU | |
| | | mCmUmUmCmGmGmAmGmGmGmAmGmCAUCAGAGAGAAGAUGGGC | |
| | | GGGAGU*mC*mU*mU | |

Note that gRNAs annotated with a v1′ design contain one less phosphorothioate bond on the 3′ end of the gRNA. gRNAs annotated with v1* contain one extra phosphorothioate bond on the 3′end of the gRNA. gRNAs annotated with a v9* contain an extra phosphorothioate bond on the 3′ end of the gRNA.

Biochemical Characterization of gRNA Activity:

Target DNA oligonucleotides with fluorescent moieties on the 5′ ends were purchased commercially (sequences listed in Table 29). Double-stranded DNA (dsDNA) targets were formed by mixing the oligos in a 1:1 ratio in 1× cleavage buffer (20 mM Tris HCl pH 7.5, 150 mM NaCl, 1 mM TCEP, 5% glycerol, 10 mM MgCl₂), following by heating to 95° C. for 10 minutes, and then allowing the solution to cool to room temperature. CasX ribonucleoproteins (RNPs) were reconstituted with CasX 491 and the indicated gRNAs at a final concentration of 1 μM with 1,2-fold excess of the indicated gRNA in 1× cleavage buffer. RNPs were allowed to form at 37° C. for 10 minutes.

The effects of various structural and chemical modifications to the gRNA scaffold on the cleavage rate of CasX 491 RNPs were determined. Cleavage reactions were prepared with final RNP concentrations of 200 nM and final target concentrations of 10 nM, and reactions were carried out at 16° C. and initiated by the addition of the labeled target DNA substrate (Table 29). Aliquots of reactions were taken at 0.25, 0.5, 1, 2, 5, and 10 minutes and quenched by adding an equal volume of 95% formamide with 20 mM EDTA. Samples were denatured at 95° C. for 10 minutes and resolved on a 10% urea-PAGE gel. Gels were imaged on a Typhoon™ laser-scanner platform and quantified using ImageQuant™ TL 8.2 image analysis software (Cytiva™). The apparent first-order rate constant of non-target strand cleavage (kcleave-) was determined for each CasX:gRNA combination.

To determine the competent fraction formed by each gRNA, cleavage reactions were prepared with final RNP concentrations of 100 nM and final target concentrations of 100 nM. Reactions were carried out at 37° C. and initiated by the addition of the labeled target substrate (Table 29). Aliquots were taken at 0.5, 1, 2, 5, 10, and 30 minutes and quenched by adding an equal volume of 95% formamide with 25 mM EDTA. Samples were denatured by heating at 95° C. for 10 minutes and resolved on a 10% urea-PAGE gel. Gels were imaged and quantified as above. CasX was assumed to act as a single-turnover enzyme under the assayed conditions, as indicated by the observation that sub-stoichiometric amounts of enzyme would fail to cleave a greater-than-stoichiometric amount of target substrate even under extended time-scales, and instead would approach a plateau that scaled with the amount of enzyme present. Thus, the fraction of target substrate cleaved over long time-scales by an equimolar amount of RNP would be indicative of the fraction of RNP that was properly formed and active for cleavage. The cleavage traces were fitted with a biphasic rate model, as the cleavage reaction clearly deviated from monophasic under this concentration regime. The plateau of each fit was determined and reported as the active fraction for each RNP in Table 32.

TABLE 29
Sequences of target DNA substrate oligonucleotides with fluorescent moieties
on the 5′ ends used for biochemical characterization of gRNA activity./700/ =
IRDye700;/800/ = IRDye800

| DNA substrate | Sequence |
|---|---|
| 6.7/6.8 target top strand | /700/catgtcttccatggccttcttcctggcttcctggtgaagatgagt |
| (SEQ ID NO: 19799) | ggcgacctgctggag |
| 6.7/6.8 target bottom strand | /800/ctccagcaggtcgccactcatcttcaccaggaagccaggaagaag |
| (SEQ ID NO: 19800) | gccatggaagacatg |

In Vitro Transcription of CasX mRNA:

DNA templates encoding for CasX 491 (see Table 30 for encoding sequences) used for in vitro transcription were generated by PCR using forward primers containing a T7 promoter, followed by agarose gel extraction of the appropriately sized DNA, 25 ng/μL final concentration of template DNA was used in each in vitro transcription reaction that was carried out following the manufacturer's recommended protocol with slight modifications. Following in vitro transcription reaction incubation for 2-3 hours at 37° C., which were carried out with CleanCap® AG and NI-methyl-pseudouridine, DNAse digestion of template DNA and column-based purification using the Zymo RNA miniprep kit were performed. The poly (A) tail was added using E. coli Poly A Polymerase following the manufacturer's protocol, followed by column-based purification as stated above. Poly (A) tailed in vitro transcribed RNA was eluted in RNAse free water, analyzed on an Agilent TapeStation for integrity, and flash frozen prior to storage at −80° C.

TABLE 30
Encoding sequences of the CasX mRNA molecules assessed in this example*

| CasX 491 | Component | | SEQ |
|---|---|---|---|
| mRNA ID | (ID) | DNA sequence | ID NO |
| CasX 491 | 5′UTR | GACCGGCCGCCACC | 19801 |
| mRNA #1 | START codon + | ATGGCCCCAGCGGCCAAACGGGTGAAGCTGGACTCTAGA | 19802 |
| | c-MYC NLS + | | |
| | linker | | |
| | CasX 491 | CAAGAGATCAAGAGAATCAACAAGATCAGAAGGAGACTGGT | 19803 |
| | | CAAGGACAGCAACACAAAGAAGGCCGGCAAGACAGGCCCCA | |
| | | TGAAAACCCTGCTCGTCAGAGTGATGACCCCTGACCTGAGA | |
| | | GAGCGGCTGGAAAACCTGAGAAAGAAGCCCGAGAACATCCC | |
| | | TCAGCCTATCAGCAACACCAGCAGGGCCAACCTGAACAAGC | |
| | | TGCTGACCGACTACACCGAGATGAAGAAAGCCATCCTGCAC | |
| | | GTGTACTGGGAAGAGTTCCAGAAAGACCCCGTGGGCCTGAT | |
| | | GAGCAGAGTTGCTCAGCCTGCCAGCAAGAAGATCGACCAGA | |
| | | ACAAGCTGAAGCCCGAGATGGACGAGAAGGGCAATCTGACC | |
| | | ACAGCCGGCTTTGCCTGCTCTCAGTGTGGCCAGCCTCTGTT | |
| | | CGTGTACAAGCTGGAACAGGTGTCCGAGAAAGGCAAGGCCT | |
| | | ACACCAACTACTTCGGCAGATGTAACGTGGCCGAGCACGAG | |
| | | AAGCTGATTCTGCTGGCCCAGCTGAAACCTGAGAAGGACTC | |
| | | TGATGAGGCCGTGACCTACAGCCTGGGCAAGTTTGGACAGA | |
| | | GAGCCCTGGACTTCTACAGCATCCACGTGACCAAAGAAAGC | |
| | | ACACACCCCGTGAAGCCCCTGGCTCAGATCGCCGGCAATAG | |
| | | ATACGCCTCTGGACCTGTGGGCAAAGCCCTGTCCGATGCCT | |
| | | GCATGGGAACAATCGCCAGCTTCCTGAGCAAGTACCAGGAC | |
| | | ATCATCATCGAGCACCAGAAGGTGGTCAAGGGCAACCAGAA | |
| | | GAGACTGGAAAGCCTGAGGGAGCTGGCCGGCAAAGAGAACC | |
| | | TGGAATACCCCAGCGTGACCCTGCCTCCTCAGCCTCACACA | |
| | | AAAGAAGGCGTGGACGCCTACAACGAAGTGATCGCCAGAGT | |
| | | GAGAATGTGGGTCAACCTGAACCTGTGGCAGAAGCTGAAAC | |
| | | TGTCCAGGGACGACGCCAAGCCTCTGCTGAGACTGAAGGGC | |
| | | TTCCCTAGCTTCCCTCTGGTGGAAAGACAGGCCAATGAAGT | |
| | | GGATTGGTGGGACATGGTCTGCAACGTGAAGAAGCTGATCA | |
| | | ACGAGAAGAAAGAGGATGGCAAGGTTTTCTGGCAGAACCTG | |
| | | GCCGGCTACAAGAGACAAGAAGCCCTGAGGCCTTACCTGAG | |
| | | CAGCGAAGAGGACCGGAAGAAGGGCAAGAAGTTCGCCAGAT | |
| | | ACCAGCTGGGCGACCTGCTGCTGCACCTGGAAAAGAAGCAC | |
| | | GGCGAGGACTGGGGCAAAGTGTACGATGAGGCCTGGGAGAG | |
| | | AATCGACAAGAAGGTGGAAGGCCTGAGCAAGCACATTAAGC | |
| | | TGGAAGAGGAAAGAAGGAGCGAGGACGCCCAATCTAAAGCC | |
| | | GCTCTGACCGATTGGCTGAGAGCCAAGGCCAGCTTTGTGAT | |
| | | CGAGGGCCTGAAAGAGGCCGACAAGGACGAGTTCTGCAGAT | |
| | | GCGAGCTGAAGCTGCAGAAGTGGTACGGCGATCTGAGAGGC | |
| | | AAGCCCTTCGCCATTGAGGCCGAGAACAGCATCCTGGACAT | |
| | | CAGCGGCTTCAGCAAGCAGTACAACTGCGCCTTCATTTGGC | |
| | | AGAAAGACGGCGTCAAGAAACTGAACCTGTACCTGATCATC | |
| | | AATTACTTCAAAGGCGGCAAGCTGCGGTTCAAGAAGATCAA | |
| | | ACCCGAGGCCTTCGAGGCTAACAGATTCTACACCGTGATCA | |
| | | ACAAAAAGTCCGGCGAGATCGTGCCCATGGAAGTGAACTTC | |
| | | AACTTCGACGACCCCAACCTGATTATCCTGCCTCTGGCCTT | |
| | | CGGCAAGAGACAGGGCAGAGAGTTCATCTGGAACGATCTGC | |
| | | TGAGCCTGGAAACCGGCTCTCTGAAGCTGGCCAATGGCAGA | |
| | | GTGATCGAGAAAACCCTGTACAACAGGAGAACCAGACAGGA | |
| | | CGAGCCTGCTCTGTTTGTGGCCCTGACCTTCGAGAGAAGAG | |
| | | AGGTGCTGGACAGCAGCAACATCAAGCCCATGAACCTGATC | |
| | | GGCGTGGACCGGGGCGAGAATATCCCTGCTGTGATCGCCCT | |
| | | GACAGACCCTGAAGGATGCCCACTGAGCAGATTCAAGGACT | |
| | | CCCTGGGCAACCCTACACACATCCTGAGAATCGGCGAGAGC | |
| | | TACAAAGAGAAGCAGAGGACAATCCAGGCCAAGAAAGAGGT | |
| | | GGAACAGCGCAGAGCCGGCGGATACTCTAGGAAGTACGCCA | |
| | | GCAAGGCCAAGAATCTGGCCGACGACATGGTCCGAAACACC | |
| | | GCCAGAGATCTGCTGTACTACGCCGTGACACAGGACGCCAT | |
| | | GCTGATCTTCGAGAATCTGAGCAGAGGCTTCGGCCGGCAGG | |
| | | GCAAGAGAACCTTTATGGCCGAGAGGCAGTACACCAGAATG | |
| | | GAAGATTGGCTCACAGCTAAACTGGCCTACGAGGGACTGAG | |
| | | CAAGACCTACCTGTCCAAAACACTGGCCCAGTATACCTCCA | |
| | | AGACCTGCAGCAATTGCGGCTTCACCATCACCAGCGCCGAC | |
| | | TACGACAGAGTGCTGGAAAAGCTCAAGAAAACCGCCACCGG | |
| | | CTGGATGACCACCATCAACGGCAAAGAGCTGAAGGTTGAGG | |
| | | GCCAGATCACCTACTACAACAGGTACAAGAGGCAGAACGTC | |
| | | GTGAAGGATCTGAGCGTGGAACTGGACAGACTGAGCGAAGA | |
| | | GAGCGTGAACAACGACATCAGCAGCTGGACAAAGGGCAGAT | |
| | | CAGGCGAGGCTCTGAGCCTGCTGAAGAAGAGGTTTAGCCAC | |
| | | AGACCTGTGCAAGAGAAGTTCGTGTGCCTGAACTGCGGCTT | |
| | | CGAGACACACGCCGATGAACAGGCTGCCCTGAACATTGCCA | |
| | | GAAGCTGGCTGTTCCTGAGAAGCCAAGAGTACAAGAAGTAC | |
| | | CAGACCAACAAGACCACCGGCAACACCGACAAGAGGGCCTT | |
| | | TGTGGAAACCTGGCAGAGCTTCTACAGAAAAAAGCTGAAAG | |
| | | AAGTCTGGAAGCCCGCCGTG | |
| | Linker + c-MYC | GGCTCCCCCGCCGCGAAGCGAGTGAAACTGGACGGATCCGG | 19804 |
| | NLS | C | |
| | P2A mScarlet + | GCAACAAACTTCTCTCTGCTGAAACAAGCCGGAGATGTCGA | 19805 |
| | STOP codon | AGAGAATCCTGGACCGATGGTATCAAAGGGTGAGGCGGTCA | |
| | | TTAAGGAATTCATGCGGTTCAAAGTTCACATGGAGGGGAGT | |
| | | ATGAATGGTCACGAATTTGAGATCGAGGGTGAGGGAGAGGG | |
| | | TAGACCTTACGAAGGCACCCAGACAGCAAAACTCAAAGTCA | |
| | | CTAAGGGCGGCCCACTCCCTTTCTCATGGGATATACTGAGT | |
| | | CCTCAATTTATGTATGGCTCCCGGGCCTTTATCAAGCATCC | |
| | | AGCCGACATACCCGATTACTATAAACAGTCATTCCCCGAGG | |
| | | GATTCAAATGGGAGAGAGTGATGAATTTTGAAGACGGGGGT | |
| | | GCCGTGACCGTAACTCAAGATACCTCTTTGGAAGATGGTAC | |
| | | CCTCATTTACAAGGTTAAGCTTAGAGGAACTAATTTCCCAC | |
| | | CAGACGGACCCGTTATGCAGAAAAAAACCATGGGTTGGGAG | |
| | | GCTAGCACCGAACGGCTGTATCCAGAGGATGGAGTTTTGAA | |
| | | AGGTGATATTAAGATGGCCCTGCGCCTGAAAGACGGAGGGC | |
| | | GGTACCTCGCAGATTTCAAAACTACCTACAAAGCTAAGAAA | |
| | | CCGGTCCAAATGCCGGGTGCTTATAACGTCGATAGGAAGCT | |
| | | GGATATAACATCACACAACGAAGACTATACGGTTGTCGAAC | |
| | | AGTATGAGCGATCTGAGGGGAGGCACAGCACGGGCGGGATG | |
| | | GATGAGCTTTACAAGTGA | |
| CasX 676 | 5′UTR + Kozak | AAATAAGAGAGAAAAGAAGAGTAAGAAGAAATATAAGAGCC | 19739 |
| mRNA #2 | sequence | ACC | |
| | (underlined) | | |
| | START codon + | ATGGCCCCTGCTGCCAAGAGAGTGAAGCTGGATAGCAGA | 19734 |
| | c-MYC NLS | | |
| | CasX 676 | CAGGAGATCAAGCGGATTAATAAAATTCGGAGAAGACTGGT | 19735 |
| | | GAAGGATTCTAACACAAAGAAGGCTGGCAAGACACGGGGCC | |
| | | CTATGAAGACACTGCTGGTGAGAGTGATGACACCCGACCTG | |
| | | AGAGAAAGACTGGAAAACCTGAGAAAGAAGCCTGAGAATAT | |
| | | CCCCCAGCCCATCAGCAACACAAGCCGGGCCAACCTGAATA | |
| | | AGCTGCTGACCGACTACACCGAAATGAAGAAGGCCATCCTG | |
| | | CACGTGTATTGGGAAGAGTTCCAGAAAGACCCAGTCGGCCT | |
| | | GATGAGCAGAGTGGCTCAGCCTGCCAGCAAGAAGATCGATC | |
| | | AGAACAAGCTGAAGCCCGAAATGGACGAGAAGGGGAACCTG | |
| | | ACAACCGCCGGCTTTGCCTGTAGCCAGTGCGGCCAGCCCCT | |
| | | GTTTGTGTACAAACTGGAACAGGTGAGCGAAAAGGGCAAGG | |
| | | CTTACACGAATTACTTCGGCAGATGCAACGTGGCCGAGCAC | |
| | | GAGAAGCTGATCAAGCTGGCCCAGCTGAAGCCTGAGAAGGA | |
| | | TAGCGATGAGGCAGTGACATATTCCCTGGGCAAGTTCGGAC | |
| | | AGCGGGCCCTGGATTTTTATTCCATTCATGTGACCAAGGAA | |
| | | TCCACCCACCCCGTCAAGCCTCTTGCCCAAATTGCCGGCAA | |
| | | CAGATACGCCTCCAGCCCCGTGGGCAAGGCCCTGAGCGACG | |
| | | CCTGTATGGGCACCATCGCCAGCTTCCTGTCTAAGTACCAG | |
| | | GACATTATCATCGAGCACCAGAAGGTGGTGAAGGGCAACCA | |
| | | GAAGAGACTGGAGAGCCTGCGCGAGCTGGCCGGCAAGGAAA | |
| | | ACCTGGAGTATCCTAGCGTGACCCTGCCTCCTCAGCCTCAT | |
| | | ACAAAGGAGGGCGTGGATGCCTACAACGAAGTGATCGCCCG | |
| | | GGTGCGGATGTGGGTGAACCTGAATCTGTGGCAGAAGCTGA | |
| | | AGCTGTCTAGAGACGACGCCAAGCCCCTGCTGAGACTGAAG | |
| | | GGCTTCCCCAGCTTCCCTCTGGTGGAGAGACAGGCAAATGA | |
| | | AGTGGACTGGTGGGACATGGTGTGTAACGTGAAGAAGCTGA | |
| | | TCAATGAGAAGAAGGAGGACGGCAAAGTGTTCTGGCAGAAT | |
| | | CTGGCCGGCTACAAGCGTCAGGAGGCCCTGCGGCCCTACCT | |
| | | GAGCAGCGAGGAAGACAGAAAGAAGGGCAAGAAGTTCGCCC | |
| | | GGTATCAGCTGGGGGACCTGCTGCTGCACCTCGAGAAGAAG | |
| | | CACGGCGAAGACTGGGGGAAGGTGTACGATGAGGCCTGGGA | |
| | | GCGGATCGATAAGAAGGTGGAGGGCCTGAGCAAGCACATCA | |
| | | AGCTGGAGGAGGAACGGAGATCTGAGGACGCCCAGAGCAAG | |
| | | GCCGCCCTGACCGACTGGCTGAGAGCCAAGGCCAGCTTCGT | |
| | | CATCGAGGGGCTGAAGGAGGCCGACAAGGACGAGTTCTGCC | |
| | | GGTGCGAACTGAAGCTGCAGAAGTGGTACGGAGATCTGAGA | |
| | | GGCAAACCTTTCGCCATCGAGGCCGAGAACAGCATCCTGGA | |
| | | CATCAGCGGCTTCAGCAAGCAGTACAACTGCGCCTTTATTT | |
| | | GGCAGAAGGACGGAGTGAAGAAGCTGAACCTGTACCTGATC | |
| | | ATCAACTATTTCAAGGGCGGCAAGCTGAGATTCAAGAAGAT | |
| | | CAAGCCTGAAGCCTTCGAGGCCAACAGATTCTACACCGTGA | |
| | | TTAACAAGAAAAGCGGAGAGATCGTGCCAATGGAAGTGAAC | |
| | | TTCAACTTCGACGACCCTAACCTGATCATCCTGCCCCTGGC | |
| | | ATTTGGCAAGCGGCAGGGCAGAGAGTTCATCTGGAACGACC | |
| | | TGCTGTCTCTGGAGACCGGCAGCCTGAAGCTGGCCAACGGC | |
| | | AGAGTGATCGAGAAGACACTGTACAACAGACGAACCAGACA | |
| | | AGACGAGCCCGCCCTGTTTGTGGCCCTGACCTTCGAGAGAA | |
| | | GAGAGGTGCTGGACAGCAGCAATATCAAGCCTATGAACCTG | |
| | | ATCGGCGTGGACCGGGGCGAGAACATCCCTGCCGTGATCGC | |
| | | CCTTACCGACCCCGAGGGATGCCCTCTGAGCCGGTTTAAAG | |
| | | ACAGCCTGGGCAACCCTACCCACATCCTGAGAATTGGCGAG | |
| | | TCCTACAAGGAGAAGCAGAGAACCATCCAGGCCAAGAAGGA | |
| | | GGTGGAGCAGCGGCGGGCTGGCGGCTACTCCCGGAAGTACG | |
| | | CCAGCAAGGCCAAGAACCTGGCCGACGACATGGTTAGAAAT | |
| | | ACCGCCAGAGACCTCCTGTACTACGCTGTGACCCAGGACGC | |
| | | CATGCTGATCTTCGAGAACCTGAGCAGAGGCTTCGGCAGAC | |
| | | AGGGCAAGAGAACCTTCATGGCCGAGAGACAGTACACCCGG | |
| | | ATGGAGGACTGGCTGACCGCCAAGCTGGCCTACGAGGGCCT | |
| | | GCCCTCTAAGACCTACCTGTCCAAGACCTTGGCACAGTACA | |
| | | CCAGCAAGACATGCTCTAACTGCGGCTTCACAATCACGAGC | |
| | | GCCGACTACGACCGGGTGCTGGAGAAACTGAAGAAGACCGC | |
| | | CACAGGCTGGATGACCACCATTAACGGCAAGGAGCTGAAGG | |
| | | TGGAGGGCCAGATCACCTACTACAACAGGTACAAACGGCAG | |
| | | AACGTGGTGAAGGACCTGAGCGTGGAACTGGATAGACTGAG | |
| | | CGAGGAAAGCGTAAACAATGACATCAGCAGCTGGACCAAGG | |
| | | GCCGGAGCGGCGAGGCCCTGAGCCTGCTGAAGAAGAGATTC | |
| | | TCCCACAGACCAGTGCAGGAGAAGTTCGTGTGTCTGAACTG | |
| | | CGGCTTCGAGACCCACGCCGACGAGCAAGCCGCCCTGAACA | |
| | | TCGCCCGGTCTTGGCTTTTCCTGCGGAGCCAGGAGTACAAG | |
| | | AAGTACCAGACAAACAAGACCACAGGCAACACAGACAAGAG | |
| | | AGCCTTCGTCGAGACCTGGCAGAGCTTCTACAGAAAGAAGC | |
| | | TGAAGGAGGTGTGGAAGCCTGCCGTG | |
| | c-MYC NLS + | GGAAGCCCCGCTGCCAAGAGAGTGAAGCTGGACTAATAGAT | 19736 |
| | STOP codons | AA | |
| | 3′UTR | GCTGCCTTCTGCGGGGCTTGCCTTCTGGCCATGCCCTTCTT | 19740 |
| | | CTCTCCCTTGCACCTGTACCTCTTGGTCTTTGAATAAAGCC | |
| | | TGAGTAGGAAG | |
| | XbaI restriction | TCTAG | 19741 |
| | site (partial) | | |
| | Poly(A) tail | AAAAAAAAAAAAAAAAAAAAAAAAAAAAAAAAAAAAAAAAA | 19738 |
| | | AAAAAAAAAAAAAAAAAAAAAAAAAAAAAAAAAAAAAAA | |

*Components are listed in a 5′ to 3′ order within the constructs

In Vitro Delivery of gRNA and CasX mRNA Via Transfection:

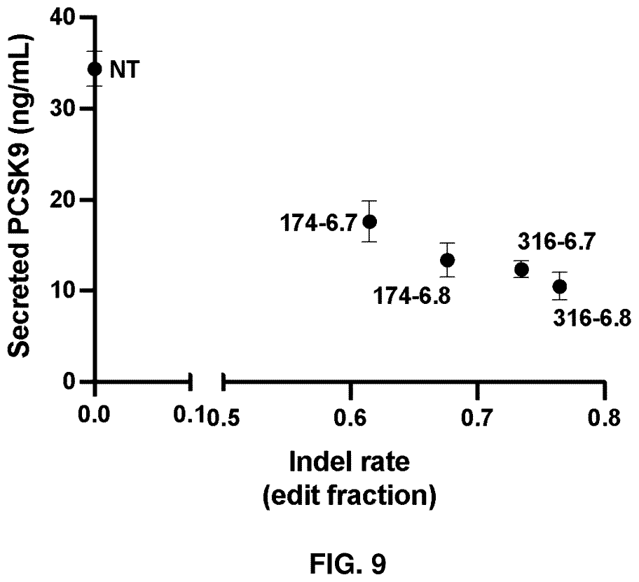

Editing at the PCSK9 locus and consequential effects on secreted PCSK9 levels were assessed for conditions using CasX 491 mRNA co-delivered with a PCSK9-targeting gRNA with scaffold variant 174 compared to conditions where a PCSK9-targeting gRNA with scaffold variant 316 was used, 100 ng of in vitro transcribed mRNA coding for CasX 491 with a P2A and mScarlet fluorescent protein was transfected into HepG2 cells with version 1 (v1) of gRNAs 174-6.7, 174-6.8, 316-6.7, and 316-6.8 (see Table 28) using lipofectamine. After a media change, the following were harvested at 28 hours post-transfection; 1) transfected cells were harvested for editing assessment at the PCSK9 locus by NGS; 2) media supernatant was harvested to measure secreted PCSK9 protein levels by ELISA. For editing analysis by NGS, amplicons were amplified from 200 ng of extracted gDNA with a set of primers targeting the PCSK9 locus and processed as described earlier in Example 5. Secreted PCSK9 levels in the media supernatant were also analyzed using a fluorescence resonance energy transfer-based immunoassay from CISBio following the manufacturer's instructions. Here, a gRNA using scaffold 174 with spacer 7.37 (v0; see Table 28), which targeted the endogenous B2M (beta-2-microglobulin) locus, served as the non-targeting (NT) control. These results are shown in FIG. 9.

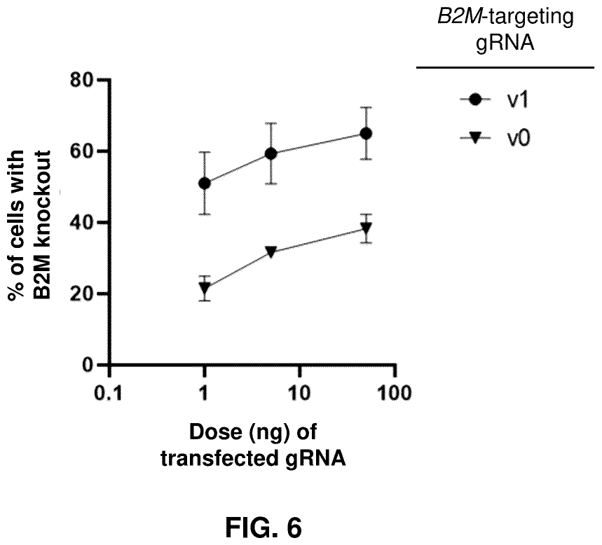

To compare the editing potency of version 0 (v0) and version 1 (v1) of B2M-targeting gRNAs, ˜6E4 HepG2 hepatocytes were seeded per well of a 96-well plate, 24 hours later, seeded cells were co-transfected using lipofectamine with 100 ng of in vitro transcribed mRNA coding for CasX 491 and different doses (1, 5, or 50 ng) of either v0 or v1 of the B2M-targeting gRNA containing scaffold variant 174 and spacer 7.37 (see Table 28). Six days post-transfection, cells were harvested for B2M protein expression analysis via immunostaining of the B2M-dependent HLA protein, followed by flow cytometry using the Attune™ NxT flow cytometer. These results are shown in FIG. 6.

V1 through v6 variants of chemically-modified PCSK9-targeting gRNAs (Table 28) were assessed for their effects on editing potency and consequential effects on secreted PCSK9 levels in vitro. Briefly, 100 ng of in vitro transcribed mRNA coding for CasX variant 491 and a P2A and mScarlet fluorescent protein was transfected into HepG2 cells with 50 ng of the indicated chemically-modified gRNA using lipofectamine. After a media change, the following were harvested at 28 hours post-transfection: 1) transfected cells for editing assessment at the PC′SK9 locus by NGS as described above: 2) media supernatant to measure secreted PCSK9 protein levels by ELISA, as described above. Here, a B2M-targeting gRNA was used as a non-targeting control. These results are shown in Table 33.

LNP co-formulations were generated as described in Example 6.

Delivery of LNPs Encapsulating CasX mRNA and Targeting gRNAs In Vitro:

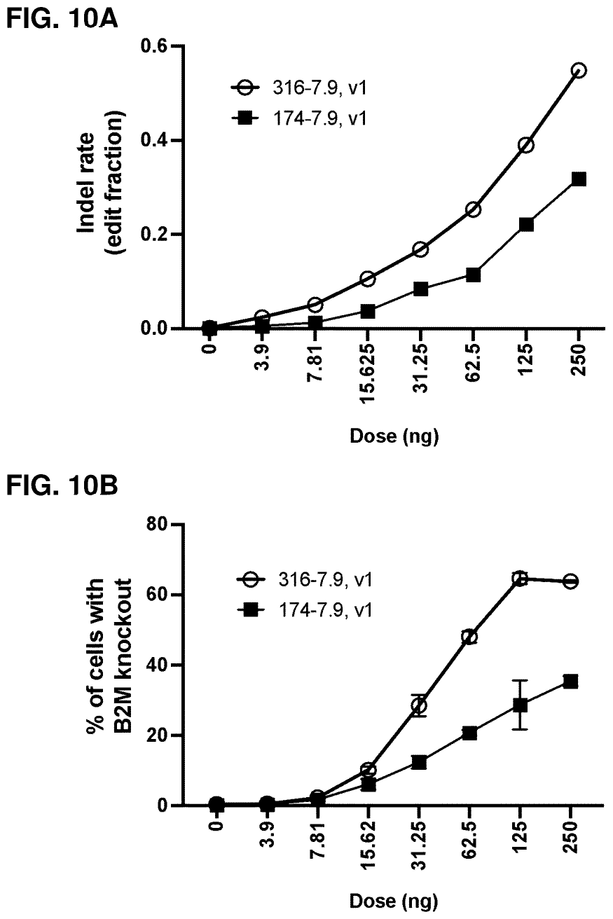

˜50,000 HepG2 cells, cultured in DMEM/F-12 media containing 10% FBS and 1% PenStrep, were seeded per well in a 96-well plate. The next day, seeded cells were treated with varying concentrations of LNPs, which were prepared in six 2-fold serial dilutions starting at 250) ng. These LNPs were formulated to encapsulate CasX 491 mRNA and a B2M-targeting gRNA incorporating either scaffold variant 174 or 316 with spacer 7.9 (v1; see Table 28). Media was changed 24 hours after LNP treatment, and cells were cultured for six additional days prior to harvesting for gDNA extraction for editing assessment at the B2M locus by NGS and B2M protein expression analysis via HLA immunostaining, followed by flow cytometry using the Attune NxT flow cytometer. Briefly, for editing assessment, amplicons were amplified from 200 ng of extracted gDNA with primers targeting the human B2M locus and processed as described in Example 5. The results of these assays are shown in FIGS. 10A and 10B.

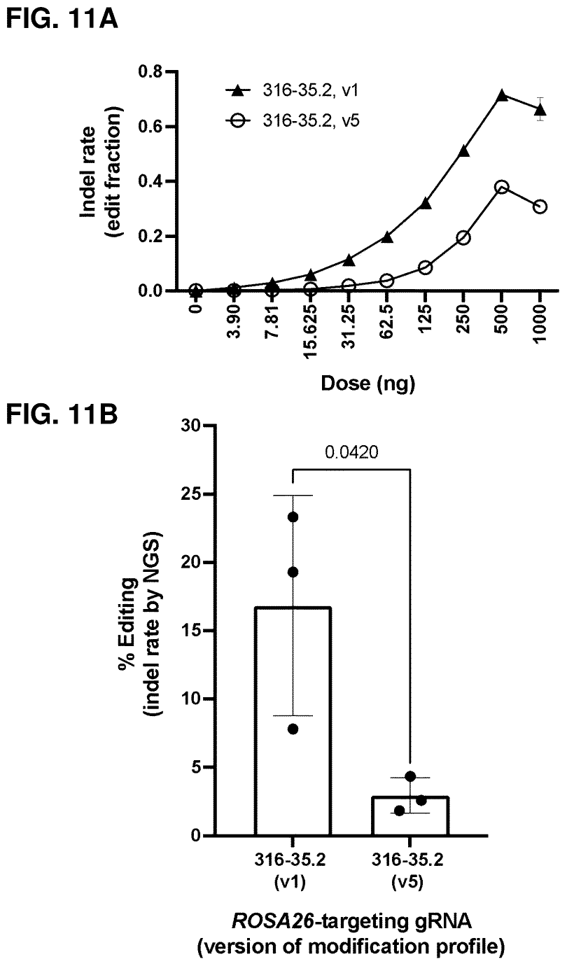

˜20,000 mouse Hepal-6 hepatocytes were seeded per well in a 96-well plate. The following day, seeded cells were treated with varying concentrations of LNPs, which were prepared in eight 2-fold serial dilutions starting at 1000 ng. These LNPs were formulated to encapsulate CasX 676 mRNA #2 (see Table 30) and a ROSA26-targeting gRNA incorporating scaffold variant 316 with spacer 35.2 (v1 or 5; see Table 28). Media was changed 24 hours post-treatment with LNPs, and cells were cultured for seven additional days prior to harvesting for gDNA extraction for editing assessment at the ROSA26 locus by NGS. Briefly, amplicons were amplified from extracted gDNA with primers targeting the mouse ROSA26 locus and processed as described in Example 5. The results of this experiment are shown in FIG. 11A.

Delivery of LNPs Encapsulating CasX mRNA and Targeting gRNA In Vivo:

To assess the effects of using v1 and v5 of scaffold 316 in vivo, CasX 676 mRNA #2 (see Table 30) and a ROSA26-targeting gRNA using scaffold 316 with spacer 35.2 (v1 or v5; see Table 28) were encapsulated within the same LNP using a 1:1 mass ratio for mRNA:gRNA. LNP co-formulations were performed as described in Example 6. Formulated LNPs were buffer-exchanged to PBS for in vivo injection. LNPs were administered intravenously through the retro-orbital sinus into 4-week old C57BL/6 mice. Mice were observed for five minutes after injection to ensure recovery from anesthesia before being placed into their home cage. Naïve, uninjected animals served as experimental controls. Six days post-administration, mice were euthanized, and the liver tissue was harvested for gDNA extraction using the Zymo Research Quick DNA/RNA Miniprep kit following the manufacturer's instructions. Target amplicons were then amplified from the extracted gDNA with a set of primers targeting the mouse ROSA26 locus and processed for editing assessment by NGS as described earlier in Example 5. The results of this experiment are shown in FIG. 11B.

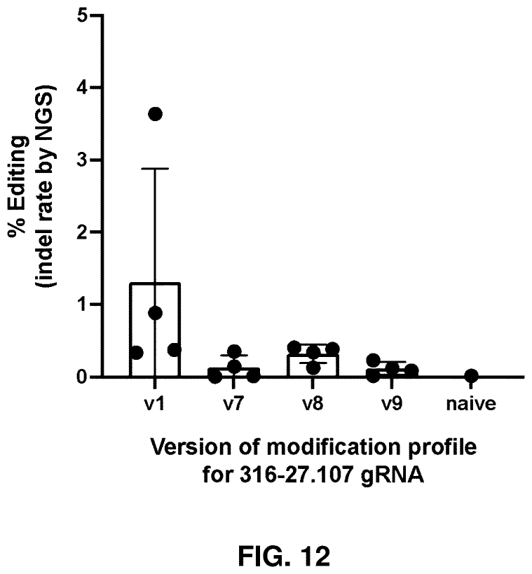

To compare the effects of using v7, v8, and v9 of scaffold 316 on editing at the PCSK9 locus in vivo, CasX 676 mRNA #1 (see Table 31 for sequences) and a PCSK9-targeting gRNA using scaffold 316 with spacer 27.107 (v1, v7, v8, or v9; see Table 28), were encapsulated within the same LNP using a 1:1 mass ratio for mRNA:gRNA for each gRNA. LNPs were administered retro-orbitally into 6-week old C57BL/6 mice, as described above, and mice were euthanized seven days post-injection to harvest liver tissue for gDNA extraction for editing assessment by NGS at the PCSK9 locus. The results of this experiment are shown in FIG. 12.

TABLE 31
Encoding sequences of CasX 676 mRNA #1 molecule

| CasX | Component | | | SEQ ID |
|---|---|---|---|---|
| ID | (ID) | Description | DNA sequence | NO |
| CasX 676 | 5′UTR | Human HBA | ACTCTTCTGGTCCCCACAGACTCAGAGAGAAC | 19733 |
| mRNA #1 | | | CCGCCACC | |
| | START | | ATGGCCCCTGCTGCCAAGAGAGTGAAGCTGGA | 19734 |
| | codon + c- | | TAGCAGA | |
| | MYC NLS | | | |
| | CasX 676 | | CAGGAGATCAAGCGGATTAATAAAATTCGGAG | 19735 |
| | | | AAGACTGGTGAAGGATTCTAACACAAAGAAGG | |
| | | | CTGGCAAGACACGGGGCCCTATGAAGACACTG | |
| | | | CTGGTGAGAGTGATGACACCCGACCTGAGAGA | |
| | | | AAGACTGGAAAACCTGAGAAAGAAGCCTGAGA | |
| | | | ATATCCCCCAGCCCATCAGCAACACAAGCCGG | |
| | | | GCCAACCTGAATAAGCTGCTGACCGACTACAC | |
| | | | CGAAATGAAGAAGGCCATCCTGCACGTGTATT | |
| | | | GGGAAGAGTTCCAGAAAGACCCAGTCGGCCTG | |
| | | | ATGAGCAGAGTGGCTCAGCCTGCCAGCAAGAA | |
| | | | GATCGATCAGAACAAGCTGAAGCCCGAAATGG | |
| | | | ACGAGAAGGGGAACCTGACAACCGCCGGCTTT | |
| | | | GCCTGTAGCCAGTGCGGCCAGCCCCTGTTTGT | |
| | | | GTACAAACTGGAACAGGTGAGCGAAAAGGGCA | |
| | | | AGGCTTACACGAATTACTTCGGCAGATGCAAC | |
| | | | GTGGCCGAGCACGAGAAGCTGATCAAGCTGGC | |
| | | | CCAGCTGAAGCCTGAGAAGGATAGCGATGAGG | |
| | | | CAGTGACATATTCCCTGGGCAAGTTCGGACAG | |
| | | | CGGGCCCTGGATTTTTATTCCATTCATGTGAC | |
| | | | CAAGGAATCCACCCACCCCGTCAAGCCTCTTG | |
| | | | CCCAAATTGCCGGCAACAGATACGCCTCCAGC | |
| | | | CCCGTGGGCAAGGCCCTGAGCGACGCCTGTAT | |
| | | | GGGCACCATCGCCAGCTTCCTGTCTAAGTACC | |
| | | | AGGACATTATCATCGAGCACCAGAAGGTGGTG | |
| | | | AAGGGCAACCAGAAGAGACTGGAGAGCCTGCG | |
| | | | CGAGCTGGCCGGCAAGGAAAACCTGGAGTATC | |
| | | | CTAGCGTGACCCTGCCTCCTCAGCCTCATACA | |
| | | | AAGGAGGGCGTGGATGCCTACAACGAAGTGAT | |
| | | | CGCCCGGGTGCGGATGTGGGTGAACCTGAATC | |
| | | | TGTGGCAGAAGCTGAAGCTGTCTAGAGACGAC | |
| | | | GCCAAGCCCCTGCTGAGACTGAAGGGCTTCCC | |
| | | | CAGCTTCCCTCTGGTGGAGAGACAGGCAAATG | |
| | | | AAGTGGACTGGTGGGACATGGTGTGTAACGTG | |
| | | | AAGAAGCTGATCAATGAGAAGAAGGAGGACGG | |
| | | | CAAAGTGTTCTGGCAGAATCTGGCCGGCTACA | |
| | | | AGCGTCAGGAGGCCCTGCGGCCCTACCTGAGC | |
| | | | AGCGAGGAAGACAGAAAGAAGGGCAAGAAGTT | |
| | | | CGCCCGGTATCAGCTGGGGGACCTGCTGCTGC | |
| | | | ACCTCGAGAAGAAGCACGGCGAAGACTGGGGG | |
| | | | AAGGTGTACGATGAGGCCTGGGAGCGGATCGA | |
| | | | TAAGAAGGTGGAGGGCCTGAGCAAGCACATCA | |
| | | | AGCTGGAGGAGGAACGGAGATCTGAGGACGCC | |
| | | | CAGAGCAAGGCCGCCCTGACCGACTGGCTGAG | |
| | | | AGCCAAGGCCAGCTTCGTCATCGAGGGGCTGA | |
| | | | AGGAGGCCGACAAGGACGAGTTCTGCCGGTGC | |
| | | | GAACTGAAGCTGCAGAAGTGGTACGGAGATCT | |
| | | | GAGAGGCAAACCTTTCGCCATCGAGGCCGAGA | |
| | | | ACAGCATCCTGGACATCAGCGGCTTCAGCAAG | |
| | | | CAGTACAACTGCGCCTTTATTTGGCAGAAGGA | |
| | | | CGGAGTGAAGAAGCTGAACCTGTACCTGATCA | |
| | | | TCAACTATTTCAAGGGCGGCAAGCTGAGATTC | |
| | | | AAGAAGATCAAGCCTGAAGCCTTCGAGGCCAA | |
| | | | CAGATTCTACACCGTGATTAACAAGAAAAGCG | |
| | | | GAGAGATCGTGCCAATGGAAGTGAACTTCAAC | |
| | | | TTCGACGACCCTAACCTGATCATCCTGCCCCT | |
| | | | GGCATTTGGCAAGCGGCAGGGCAGAGAGTTCA | |
| | | | TCTGGAACGACCTGCTGTCTCTGGAGACCGGC | |
| | | | AGCCTGAAGCTGGCCAACGGCAGAGTGATCGA | |
| | | | GAAGACACTGTACAACAGACGAACCAGACAAG | |
| | | | ACGAGCCCGCCCTGTTTGTGGCCCTGACCTTC | |
| | | | GAGAGAAGAGAGGTGCTGGACAGCAGCAATAT | |
| | | | CAAGCCTATGAACCTGATCGGCGTGGACCGGG | |
| | | | GCGAGAACATCCCTGCCGTGATCGCCCTTACC | |
| | | | GACCCCGAGGGATGCCCTCTGAGCCGGTTTAA | |
| | | | AGACAGCCTGGGCAACCCTACCCACATCCTGA | |
| | | | GAATTGGCGAGTCCTACAAGGAGAAGCAGAGA | |
| | | | ACCATCCAGGCCAAGAAGGAGGTGGAGCAGCG | |
| | | | GCGGGCTGGCGGCTACTCCCGGAAGTACGCCA | |
| | | | GCAAGGCCAAGAACCTGGCCGACGACATGGTT | |
| | | | AGAAATACCGCCAGAGACCTCCTGTACTACGC | |
| | | | TGTGACCCAGGACGCCATGCTGATCTTCGAGA | |
| | | | ACCTGAGCAGAGGCTTCGGCAGACAGGGCAAG | |
| | | | AGAACCTTCATGGCCGAGAGACAGTACACCCG | |
| | | | GATGGAGGACTGGCTGACCGCCAAGCTGGCCT | |
| | | | ACGAGGGCCTGCCCTCTAAGACCTACCTGTCC | |
| | | | AAGACCTTGGCACAGTACACCAGCAAGACATG | |
| | | | CTCTAACTGCGGCTTCACAATCACGAGCGCCG | |
| | | | ACTACGACCGGGTGCTGGAGAAACTGAAGAAG | |
| | | | ACCGCCACAGGCTGGATGACCACCATTAACGG | |
| | | | CAAGGAGCTGAAGGTGGAGGGCCAGATCACCT | |
| | | | ACTACAACAGGTACAAACGGCAGAACGTGGTG | |
| | | | AAGGACCTGAGCGTGGAACTGGATAGACTGAG | |
| | | | CGAGGAAAGCGTAAACAATGACATCAGCAGCT | |
| | | | GGACCAAGGGCCGGAGCGGCGAGGCCCTGAGC | |
| | | | CTGCTGAAGAAGAGATTCTCCCACAGACCAGT | |
| | | | GCAGGAGAAGTTCGTGTGTCTGAACTGCGGCT | |
| | | | TCGAGACCCACGCCGACGAGCAAGCCGCCCTG | |
| | | | AACATCGCCCGGTCTTGGCTTTTCCTGCGGAG | |
| | | | CCAGGAGTACAAGAAGTACCAGACAAACAAGA | |
| | | | CCACAGGCAACACAGACAAGAGAGCCTTCGTC | |
| | | | GAGACCTGGCAGAGCTTCTACAGAAAGAAGCT | |
| | | | GAAGGAGGTGTGGAAGCCTGCCGTG | |
| | c-MYC | | GGAAGCCCCGCTGCCAAGAGAGTGAAGCTGGA | 19736 |
| | NLS + | | CTAATAGATAA | |
| | STOP codons | | | |
| | 3′UTR | Human HBA | GCTGGAGCCTCGGTGGCCATGCTTCTTGCCCC | 19737 |
| | | | TTGGGCCTCCCCCCAGCCCCTCCTCCCCTTCC | |
| | | | TGCACCCGTACCCCCGTGGTCTTTGAATAAAG | |
| | | | TCTGAGTGGGCGGCA | |
| | Poly(A) tail | | AAAAAAAAAAAAAAAAAAAAAAAAAAAAAAAA | 19738 |
| | | | AAAAAAAAAAAAAAAAAAAAAAAAAAAAAAAA | |
| | | | AAAAAAAAAAAAAAAA | |

*Components are listed in a 5′ to 3′ order within the constructs

### Results

Assessing the Effects of Various Chemical Modifications on gRNA Activity:

Several studies involving Cas9 have demonstrated that chemical modifications of the gRNA resulted in significantly improved editing activity when delivered with Cas9 mRNA. Following delivery of Cas9 mRNA and gRNA into target cells, unprotected gRNA is susceptible to degradation during the mRNA translation process. Addition of chemical modifications such as 2′O-methyl (2′OMe) groups and phosphorothioate bonds can reduce the susceptibility of the gRNA to cellular RNases, but also have the potential to disrupt folding of the gRNA and its interactions with the CRISPR-Cas protein. Given the lack of structural similarity between CasX and Cas9, as well as their respective gRNAs, appropriate chemical modification profiles must be designed and validated de novo. Using published structures of wild-type CasX from Deltaproteobacteria (PDB codes 6NY1, 6NY2, and 6NY3) as reference, residues that appeared potentially amenable to modification were selected. However, the published structures were of a wild type CasX ortholog and gRNA distinct from the species used as the basis for the engineered variants presented here, and they also lacked the resolution to confidently determine interactions between protein side-chains and the RNA backbone. These limitations introduced a significant amount of ambiguity into determining which nucleotides might be safely modified. As a result, six profiles of chemical modifications (denoted as versions) were designed for initial testing, and these six profiles are illustrated in FIGS. 5A and 5B. The v1 profile was designed as a simple end-protected structure, where the first and last three nucleotides were modified with 2′OMe and phosphorothioate bonds. In the v2 profile, 3′UUU tail was added to mimic the termination sequence used in cellular transcription systems and to move the modified nucleotides outside of the region of the spacer involved in target recognition. The v3 profile included the end protection as in v1, as well as the addition of 2′OMe modifications at all nucleotides identified to be potentially modifiable based on structural analysis. The v4 profile was modeled based on v3, but with all the modifications in the triplex region removed, as this structure was predicted to be more sensitive to any perturbation of the RNA helical structure and backbone flexibility. The v5 profile maintained chemical modifications in the scaffold stem and extended stem regions, while the v6 profile harbored modifications only in the extended stem. The extended stem is a region that would become fully exposed to solvent in the RNP and is amenable to replacement by other hairpin structures, and therefore presumably relatively insensitive to chemical modifications.

The minimally modified v1 gRNA was initially assessed against an unmodified gRNA (v0) to determine the potential benefit of such chemical modifications on editing when the gRNA was co-delivered with CasX mRNA to target cells. Modified (v1) and unmodified (v0) B2M-targeting gRNAs with spacer 7.37 were co-transfected with CasX mRNA into HepG2 cells, and editing at the B2M locus was measured by loss of surface presentation of the B2M-dependent HLA complex, as detected by flow cytometry (FIG. 6). The data demonstrate that use of the v1 gRNA resulted in substantially greater loss of B2M expression compared to the levels seen with v0 gRNA across the various doses, thereby confirming that end modifications of the gRNA increased CasX-mediated editing activity upon delivery of the CasX mRNA and gRNA.

The broader set of gRNA chemical modification profiles were assessed using PCSK9-targeting gRNAs using scaffold variant 235 and spacers 6.7 and 6.8 to determine whether the additional chemical modifications would be able to support the formation of active RNPs. In vitro cleavage assays described above were performed to determine keleave and fraction competence for these engineered gRNAs harboring the various chemical modification profiles. The results from these in vitro cleavage assays are shown in Table 32. The data demonstrate that gRNAs with the v3 profiles exhibited no activity, an indication that the addition of some chemical modifications significantly interfered with RNP formation or activity. Adding v4 chemical modifications resulted in a reasonable cleavage rate in the excess RNP condition, but exhibited very low fraction competence. The difference between v3 and v4 modifications confirmed that modifications in the triplex region prevented the formation of any active RNP, either due to the inability of the gRNA to fold properly or a disruption in the gRNA-protein interactions. The reduced fraction competence resulting from appending v4 modifications suggest that while the gRNA was able to successfully assemble with the CasX protein to form a cleavage-competent RNP, a large majority of the gRNA was misfolded, or that the appended chemical modifications reduced the affinity of the gRNA for the CasX protein and impeded the efficiency of RNP formation. Application of the v5 or v6 profiles resulted in competent fractions that were comparable to, but slightly lower than, those obtained for reactions using the v1 and v2 modifications. While the kcleave values were relatively consistent between v5 and v6 gRNAs, both v5 and v6 gRNAs achieved nearly half of the keleave values for v1 and v2 gRNAs. The reduced keleave value for v6 gRNA was particularly surprising, given the lack of expected interaction between the gRNA and CasX protein in the modified extended stem. However, for both v5 and v6 gRNAs, it is possible that the reduced flexibility of the gRNA, resulting from the 2′OMe modifications, inhibited structural changes in the RNP required for efficient cleavage, or that the modified initial base-pairs of the hairpin involved in CasX protein interaction had been negatively impacted by the inclusion of the 2′OMe groups.

TABLE 32
Parameters of cleavage activity assessed for CasX
RNPs with the various PCSK9-targeting gRNAs using
scaffold 235 and harboring the indicated chemical
modification profile, denoted by version number.

| gRNA | | |
|---|---|---|
| (scaffold variant-spacer, | k_{cleave} | Fraction |
| version no.) | (min⁻¹) | competence |
| 235-6.7, v1 | 0.901 | 0.398 |
| 235-6.8, v1 | 1.36 | 0.398 |
| 235-6.7, v2 | 0.454 | 0.386 |
| 235-6.8, v2 | 2.03 | 0.361 |
| 235-6.7, v3 | 0 | 0 |
| 235-6.8, v3 | 0 | 0 |
| 235-6.7, v4 | 0.434 | 0.031 |
| 235-6.8, v4 | 0.257 | 0.005 |
| 235-6.7, v5 | 0.506 | 0.313 |
| 235-6.8, v5 | 0.680 | 0.388 |
| 235-6.7, v6 | 0.462 | 0.346 |
| 235-6.8, v6 | 0.715 | 0.325 |

The chemically-modified PCSK9-targeting gRNAs based on scaffold 235 were subsequently assessed for editing in a cell-based assay. CasX mRNA and chemically modified PCSK9-targeting gRNAs were co-transfected into HepG2 cells using lipofectamine. Editing levels were measured by indel rate at the PCSK9 locus by NGS and secreted PCSK9 levels by ELISA, and the data are displayed in Table 33. The data demonstrate that use of v3 and v4 gRNAs resulted in minimal editing activity at the PCSK9 locus, consistent with findings from the biochemical in vitro cleavage assays shown in Table 32. Meanwhile, use of v5 and v6 gRNAs resulted in editing levels, measured by indel rate and PCSK9 secretion, that were slightly lower than the levels attained with use of v1 and v2 gRNAs (Table 33). Specifically, the results show that use of v1 and v2 gRNAs, which harbored end modifications, resulted in ˜80-85% editing at the PCSK9 locus, indicating that adding chemical modifications to the gRNA ends was sufficient to achieve efficient editing with CasX. While the data demonstrate that use of v5 and v6 gRNAs resulted in efficient editing in vitro, near-saturating levels of editing were observed with use of the v1 gRNA in this experiment where a single dose of the gRNA was transfected. As a result, the use of a single dose rendered it challenging to assess clearly the effects of the chemical modifications on editing under guide-limiting conditions. Therefore, profiles v1 and v5 were chosen for further testing, as v1 contains the simplest modification profile, and v5 is the most heavily modified profile whose application demonstrated robust activity in vitro (Tables 7F and 7G).

TABLE 33
Editing levels measured by indel rate at PCSK9 locus by NGS and
secreted PCSK9 levels by ELISA in HepG2 cells co-transfected
with CasX 491 mRNA and various chemically-modified PCSK9-targeting
gRNAs using scaffold 235 and either spacer 6.7 or 6.8.

| Experimental | Indel rate (edit fraction) | | Secreted PCSK9 (ng/mL) | |
|---|---|---|---|---|
| condition | Mean | Stdev | Mean | Stdev |
| CasX mRNA only | 0.0021 | 0.003 | 52 | 14 |
| 235-6.7, v1 | 0.83 | 0.0058 | 18 | 5.7 |
| 235-6.7, v2 | 0.79 | 0.0071 | 21 | 4 |
| 235-6.7, v3 | 0.024 | 0.02 | 48 | 19 |
| 235-6.7, v4 | 0.12 | 0.006 | 34 | 5.5 |
| 235-6.7, v5 | 0.73 | 0.023 | 21 | 9 |
| 235-6.7, v6 | 0.75 | 0.0069 | 22 | 8.8 |
| 235-6.8, v1 | 0.85 | 0.017 | 16 | 4.4 |
| 235-6.8, v2 | 0.83 | 0.0028 | 20 | 1.5 |
| 235-6.8, v3 | 0.023 | 0.0027 | 39 | 2.7 |
| 235-6.8, v4 | 0.088 | 0.0086 | 42 | 10 |
| 235-6.8, v5 | 0.77 | 0.017 | 19 | 1.6 |
| 235-6.8, v6 | 0.78 | 0.014 | 24 | 6.9 |
| Non-targeting ctrl | 0.0019 | 0.0026 | 42 | 12 |

The v1 and v5 profiles were further tested in another cell-based assay to assess their effects on editing efficiency. LNPs were formulated to co-encapsulate CasX 676 mRNA #2 and v1 and v5 chemically-modified ROSA26-targeting gRNAs using the newly-designed gRNA scaffold 316 (described further in the following sub-section). The “v5” profile was modified slightly for application to the 316 scaffold. Three 2′ OMe modifications in the non-base-paired region immediately 5′ of the extended stem were removed to restrict modifications to the two stemloop regions. Hepal-6 hepatocytes were treated with the resulting LNPs at various doses and harvested eight days post-treatment to assess editing at the ROSA26 locus, measured as indel rate detected by NGS (FIG. 11A). The data demonstrate that treatment with LNPs delivering the v5 ROSA26-targeting gRNA resulted in markedly lower editing levels across the range of doses compared to the levels achieved with the v1 counterpart (FIG. 11A). There are several possible explanations for the differences in relative activity observed with use of v5 gRNA in FIG. 11A relative to that observed in Table 33. The first and most likely possible explanation is that the single dose used to achieve editing shown in Table 33 was too high to measure differences in activity accurately between use of v5 gRNA and v1 gRNA. It is also possible that the removal of the modifications outside the stemloop motifs in the 316 version of v5 negatively impacted guide activity. While it is possible that these modifications provide stability benefits that outweigh an activity cost imparted by the stemloop modifications, this seems unlikely given that increasing levels of modification have so far resulted in decreased activity. A final possible explanation is that the modifications in the v5 profile might negatively impact LNP formulation or behavior through differential interactions between the modified nucleotide backbone and the ionizable lipid of the LNP, potentially resulting in less efficient gRNA encapsulation or in less efficient gRNA release following internalization.

LNPs co-encapsulating the CasX mRNA #2 and v1 and v5 chemically-modified ROSA26-targeting gRNAs based on scaffold 316 were further tested in vivo. FIG. 11B shows the results of the editing assay as percent editing measured as indel rate at the ROSA26 locus. The data demonstrate that use of the v5 gRNA resulted in ˜5-fold lower editing compared to that achieved with use of the v1 gRNA, under more relevant testing conditions of in vivo LNP delivery. These findings support the reduced cleavage rate observed biochemically for the v5 gRNA in Table 32, an indication that the v5 modifications have interfered with some aspect of CasX activity. Given the consistent decrease in activity detected in v5 and v6 profiles (Table 32), the reduced editing may be attributed to modifications in the extended stem region. Although the extended stem of the gRNA has minimal interactions with the CasX protein, it is possible that addition of 2 OMe groups at the first base-pair disrupted either the CasX protein-gRNA interactions or the complex RNA fold where the extended stem meets the pseudoknot and triplex regions. More specifically, inclusion of the 2′OMe groups might have adversely affected the basal base-pairs of the gRNA extended stem and residues R49, K50, and K51 of the CasX protein. Finally, structural studies of CasX have suggested that flexibility of the gRNA is required for efficient DNA cleavage (Liu J, et al, CasX enzymes comprise a distinct family of RNA-guided genome editors. Nature 566:218-223 (2019); Tsuchida C A, et al, Chimeric CRISPR-CasX enzymes and guide RNAs for improved genome editing activity. Mol Cell 82 (6): 1199-1209 (2022)). Thus, the addition of the 2′OMe groups throughout the extended stem might have enforced a more rigid A-form helical structure and prevented the needed flexibility for the gRNA for efficient cleavage. Furthermore, it is possible that the additional modifications in the scaffold stem in the v5 and v6 profiles might be detrimental to activity, though this is currently unclear given the limited comparisons between the v5 and v6 profiles.

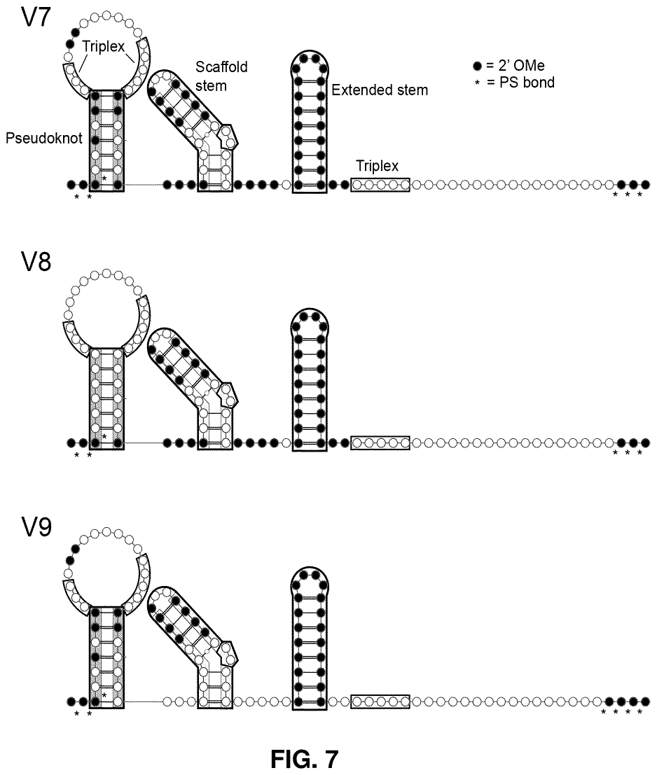

Additional modification profiles were designed with the goal of enhancing gRNA stability while mitigating the adverse effects on RNP cleavage activity. Using recently published structures of wild-type CasX from Planctomycetes (PDB codes 7WAY, 7WAZ, 7WB0, 7WB1), which has a higher homology to the engineered CasX variants being assessed, additional chemical modification profiles for gRNAs were designed and are illustrated in FIG. 7. These profiles illustrate the addition of 2′OMe groups and phosphorothioate bonds to a newly-designed gRNA scaffold variant, which is described in the ensuing sub-section. These new gRNA chemical modification profiles were designed based on the initial data demonstrating sufficient editing activity observed in Table 33 with use of the v5 gRNA that suggested that modifications to the extended stem and scaffold stem regions would not negatively impact activity. The v7 profile was designed to include 2′OMe at residues likely to be modifiable throughout the gRNA structure, which excluded the triplex region, given the dramatic negative effects of adding such modifications observed earlier with the v3 profile. More conservative profiles, v8 and v9, were also designed, as illustrated in FIG. 7. For the v8 construct, modifications were removed in the pseudoknot and triplex loop region, but were retained in the scaffold stem, extended stem, and their flanking single-stranded regions, in addition to the 5′ and 3′ termini. For the v9 profile, modifications were removed in the single-stranded regions flanking the stemloops, but were retained in the stemloops themselves, in addition to the pseudoknot, triplex loop, and 5′ and 3′ termini. The additional chemical modification profiles v7, v8, and v9 of the newly designed gRNA scaffold variant 316 (discussed further below) were assessed in vivo at the PCSK9 locus. The results of the editing assay in vivo quantified as percent editing at the PC′SK9 locus measured as indel rate as detected NGS are illustrated in FIG. 12. Despite the fact that low editing efficiency was detected overall, the data demonstrate that use of v7, v8, and v9 gRNAs resulted in lower editing levels at the PCSK9 locus compared to the indel rate achieved with use of the v1 gRNA (FIG. 12). Given the findings in FIGS. 11A-11B showing inferior editing activity attained with the v5 gRNA, it is unsurprising that v7, v8, and v9 profiles similarly demonstrated comparatively lower editing activity. As illustrated in FIG. 7, the v7, v8, and v9 profiles include modifications throughout the extended stem region, which might have interfered with RNP activity.

Comparison of gRNA Scaffold Variant 174 and 316 Using an In Vitro Cleavage Assay:

Previous work had established gRNA scaffold variant 235 as the top-performing scaffold variant across multiple delivery conditions. However, the longer length of scaffold 235 (119 bp, when using a 20 bp spacer) relative to gRNAs including scaffold 174 (109 bp, when using a 20 bp spacer) increased the difficulty of solid-phase RNA synthesis, which would result in increased manufacturing costs, decreased purity and vield, and higher rates of synthesis failures. To address these issues but retain the improved activity of using scaffold variant 235, a chimeric gRNA scaffold was designed primarily on the basis of the scaffold 235 sequence, but the extended stemloop of scaffold 235 was replaced with the shorter extended stemloop of scaffold variant 174 (FIGS. 8A-8C). The resulting chimeric scaffold, named scaffold 316, was synthesized in parallel with scaffold 174 and PCSK9-targeting spacers 6.7 and 6.8, and B2M-targeting spacer 7.9 harboring the v1 chemical modification profile, with 2′OMe and phosphorothioate bonds on the first and last three nucleotides of all gRNAs (see Table 28). Scaffold variant 174 was chosen as the comparator rather than variant 235 because variant 174 was the best previously characterized scaffold with the same length as variant 316.

In vitro cleavage activity was assessed for gRNAs with scaffold 174 and 316 and spacers 6.7 and 6.8. Cleavage assays were carried out with 20-fold excess RNP over a matching dsDNA target. Cleavage rates were quantified for all four guides, and the results are shown in Table 34. The data demonstrate that in the context of spacer 6.7, use of either scaffold 174 or 316 resulted in similar cleavage rates, with scaffold 316 resulting in marginally faster cleavage than that achieved with scaffold 174. In the context of spacer 6.8, the difference in cleavage activity was more pronounced: CasX RNPs using scaffold 316 were able to cleave DNA nearly twice as quickly as CasX RNPs using scaffold 174 (Table 34).

Assays were also performed with equimolar amounts of RNP and DNA target over a longer time course to assess the fraction of expected RNP active for cleavage. As the CasX RNP is essentially single-turnover over the tested timescale, and the concentrations used are expected to be substantially higher than the Kp of the DNA-binding reaction, the amount of cleaved DNA should approximate the amount of active RNP. For either spacer 6.7 or 6.8, the active fraction of CasX RNPs incorporating scaffold 316 was 25-30% higher than for CasX RNPs using scaffold 174 (Table 34). These data suggest that a higher fraction of gRNA using scaffold 316 was properly folded for association with the CasX protein, or that the gRNA using scaffold 316 was able to associate more strongly with the CasX protein. Compared to scaffold 174, scaffold 316 harbors mutations expected to stabilize the pseudoknot and triplex structures required for proper gRNA folding. The increased stability of these motifs in particular, which were more likely to misfold than the simple hairpins found elsewhere in the gRNA structure, might result in a slightly higher fraction of the gRNAs folding into an active conformation.

TABLE 34
Parameters of cleavage activity assessed for CasX RNPs
with gRNAs containing scaffold variant 174 or 316 with
the version 1 (v1) chemical modification profile.

| gRNA | k_{cleave} | Fraction |
|---|---|---|
| (scaffold variant-spacer) | (min⁻¹) | competence |
| 174-6.7, v1 | 0.236 | 0.194 |
| 174-6.8, v1 | 0.142 | 0.165 |
| 316-6.7, v1 | 0.264 | 0.244 |
| 316-6.8, v1 | 0.272 | 0.213 |

Comparison of gRNA Scaffold Variant 174 and 316 in a Cell-Based Assay:

An editing assessment using gRNA scaffold variant 174 compared to variant 316 was performed in a cell-based assay. CasX 491 mRNA and the version 1 (v1) of PCSK9-targeting gRNAs using spacers 6.7 and 6.8 were lipofected into HepG2 cells. Treated cells were harvested 28 hours post-transfection for analysis of editing levels at the PCSK9 locus by NGS and secreted PCSK9 levels by ELISA, and the data are presented in FIG. 9. The data demonstrate that use of any of the PCSK9-targeting gRNA tested resulted in efficient editing at the PCSK9 locus and substantial reduction in PCSK9 secretion compared to the non-targeting control using the B2M-targeting gRNA. The results also show that use of scaffold 316 resulted in more effective editing at the PCSK9 locus than that observed with use of scaffold 174 (˜10 percentage point increase in editing rate achieved with scaffold 316 over scaffold 174). This finding is further supported by the ELISA results, such that use of scaffold 316 resulted in more effective reduction of PCSK9 secretion compared to that achieved with use of scaffold 174.

Scaffold variants 174 and 316 were also assessed in an editing assay where LNPs were formulated to co-encapsulate CasX 491 mRNA and B2M-targeting gRNA harboring either scaffold variant. HepG2 cells were treated with the resulting LNPs at various doses and harvested seven days post-treatment to assess editing at the B2M locus, measured as indel rate detected by NGS (FIG. 10A) and loss of surface presentation of the B2M-dependent HLA complex, as detected by flow cytometry (FIG. 10B). The results from both assays demonstrate that treatment with LNPs to deliver the B2M-targeting gRNA using scaffold 316 resulted in higher editing potency at the B2M locus compared to LNPs delivering the gRNA using scaffold 174 at each dose (FIGS. 10A and 10B). Specifically, at the highest dose of 250 ng, use of scaffold 316 resulted in an editing level that was nearly two-fold higher than the level attained with using scaffold 174. This substantial increase in editing efficacy when using scaffold 316 versus scaffold 174, compared to the comparatively modest difference in activity observed from the in vitro cleavage assays, might be attributed to the destabilization of gRNA structure and folding during LNP formulation. The low pH conditions and association of cationic lipids during LNP formulation could adversely affect parts of the gRNA structure and result in unfolding. Consequently, it would be necessary for the gRNA to refold quickly in the cytoplasm upon delivery, both to bind the CasX protein to form the RNP and to evade RNase degradation. The stability-increasing mutations in scaffold 316 compared to scaffold 174 might provide a substantial benefit in supporting proper gRNA refolding in the cytoplasm after LNP delivery, while the deliberate folding protocol carried out for the gRNA prior to biochemical experiments likely reduced the impact of these mutations.

### Example 8: CasX:gRNA In Vitro Cleavage Assays

Experiments were performed to assess in vitro DNA cleavage by CasX:gRNA ribonucleoproteins (RNPs).

### Materials and Methods

RNPs of either CasX 119, CasX 491 (SEQ ID NO: 4), CasX 515 (SEQ ID NO: 5), or CasX 812 (SEQ ID NO: 7) were assembled with single guide RNAs (sgRNA) with scaffold 316 (ACUGGCGCUUCUAUCUGAUUACUCUGAGCGCCAUCACCAGCGACUAUGUCGU AGUGGGUAAAGCUCCCUCUUCGGAGGGAGCAUCAGAG; SEQ ID NO: 466) and one of two spacers, as described in detail below. The amino acid sequences of CasX 119 and CasX 491 are disclosed in International Publication No. WO2020247882A1, and the amino acid sequence of CasX 515 is disclosed in International Publication No. WO2022120095A1. Separately, RNPs of CasX 515 were assembled with sgRNA with either scaffold 2, 174, 235, or 316 and one of two spacers. The RNA sequences of scaffolds 2 and 174 are disclosed in International Publication No. WO2020247882A1, and the RNA sequence of scaffold 235 is disclosed in International Publication No. WO2022120095A1.

Purified RNP of CasX and sgRNA were prepared same-day prior to experiments. For experiments where protein variants were being compared, the CasX protein was incubated with sgRNA at 1:1.2 molar ratio. When scaffolds were compared, the protein was added in 1.2:1 ratio to guide. Briefly, sgRNA was added to Buffer #1 (20 mM Tris HCl pH 7.5, 150 mM NaCl, 1 mM TCEP, 5% glycerol, 10 mM MgCl₂) on ice, then CasX was added to the sgRNA solution, slowly with swirling, and immediately incubated at 37° C. for 20 minutes to form RNP complexes. RNP complexes were centrifuged at 4° C. for 5 minutes at 16,000×g to remove any precipitate. Formation of competent (active) RNP was assessed as described below.

### In Vitro Cleavage Assays:

The ability of CasX variants to form active RNP compared to reference CasX was determined using an in vitro cleavage assay. The beta-2 microglobulin (B2M) 7.9 and 7.37 target for the cleavage assay was created as follows. DNA oligos (sequences in Table 35) were generated with 5′ terminal amino modification for conjugation to Cy-dyes with an amino-reactive handle (N-hydroxysuccinimide). Oligo-dye conjugation reactions of 100 uM oligo and 1 mM dye were performed in 100 mM sodium borate pH 8.3 at 4° C. for 16 h. Target strands (TS) were labeled with Cy5.5 and non-targeting strands (NTS) were labeled with Cy7.5. After quenching the reactions with 1 mM Tris pH 7.5, the conjugated oligos were purified via ethanol precipitation. Double-stranded DNA (dsDNA) targets were formed by mixing the oligos in a 1:1 ratio in 1× hybridization buffer (20 mM Tris HCl pH 7.5, 100 mM KCl, 5 mM MgCl₂), heating to 95° C. for 10 minutes, and allowing the solution to cool to room temperature.

TABLE 35
DNA sequences and descriptions of target DNAs

| DNA Sequence (5′-3′)* | SEQ ID NO | Description |
|---|---|---|
| /5AmMC6/TGAAGCTGACAGCATTCGGGCCGAGA | 20025 | 7.37 NTS with 5′ amine for NHS |
| TGTCTCGCTCCGTGGCCTTAGCTGTGCTCGCGCT | | ester linkages** |
| /5AmMC6/AGCGCGAGCACAGCTAAGGCCACGGA | 20026 | 7.37 TS with 5′ amine* |
| GCGAGACATCTCGGCCCGAATGCTGTCAGCTTCA | | |
| /5AmMC6/TGAAGCTGACAGCATTCGGGCCTAGA | 20027 | 7.37 gel probe NTS, mismatch at 5 |
| TGTCTCGCTCCGTGGCCTTAGCTGTGCTCGCGCT | | |
| /5AmMC6/AGCGCGAGCACAGCTAAGGCCACGGA | 20028 | 7.37 gel probe TS, mismatch at 5 |
| GCGAGACATCTAGGCCCGAATGCTGTCAGCTTCA | | |
| /5AmMC6/TGAAGCTGACAGCATTCGGGCCGAGA | 20029 | 7.37 gel probe NTS, mismatch at 10 |
| TATCTCGCTCCGTGGCCTTAGCTGTGCTCGCGCT | | |
| /5AmMC6/AGCGCGAGCACAGCTAAGGCCACGGA | 20030 | 7.37 gel probe TS, mismatch at 10 |
| GCGAGATATCTCGGCCCGAATGCTGTCAGCTTCA | | |
| /5AmMC6/TGAAGCTGACAGCATTCGGGCCGAGA | 20031 | 7.37 gel probe NTS, mismatch at 15 |
| TGTCTCGATCCGTGGCCTTAGCTGTGCTCGCGCT | | |
| /5AmMC6/AGCGCGAGCACAGCTAAGGCCACGGA | 20032 | 7.37 gel probe TS, mismatch at 15 |
| TCGAGACATCTCGGCCCGAATGCTGTCAGCTTCA | | |
| /5AmMC6/CTTTCAGCAAGGACTGGTCTTTCTAT | 20033 | 7.9 target TS |
| CTCTTGTACTACACTGAATTCACCCCCACTGAAA | | |
| /5AmMC6/TTTCAGTGGGGGTGAATTCAGTGTAG | 20034 | 7.9 target NTS |
| TACAAGAGATAGAAAGACCAGTCCTTGCTGAAAG | | |
| /5AmMC6/CTTTCAGCAAGGACTGGTCTTTCTAT | 20035 | 7.9 gel probe TS, mismatch at 5 |
| CTCTTGTACGACACTGAATTCACCCCCACTGAAA | | |
| /5AmMC6/TTTCAGTGGGGGTGAATTCAGTGTCG | 20036 | 7.9 gel probe NTS, mismatch at 5 |
| TACAAGAGATAGAAAGACCAGTCCTTGCTGAAAG | | |
| /5AmMC6/CTTTCAGCAAGGACTGGTCTTTCTAT | 20037 | 7.9 gel probe TS, mismatch at 10 |
| CTCTCGTACTACACTGAATTCACCCCCACTGAAA | | |
| /5AmMC6/TTTCAGTGGGGGTGAATTCAGTGTAG | 20038 | 7.9 gel probe NTS, mismatch at 10 |
| TACGAGAGATAGAAAGACCAGTCCTTGCTGAAAG | | |
| /5AmMC6/CTTTCAGCAAGGACTGGTCTTTCTGT | 20039 | 7.9 gel probe TS, mismatch at 15 |
| CTCTTGTACTACACTGAATTCACCCCCACTGAAA | | |
| /5AmMC6/TTTCAGTGGGGGTGAATTCAGTGTAG | 20040 | 7.9 gel probe NTS, mismatch at 15 |
| TACAAGAGACAGAAAGACCAGTCCTTGCTGAAAG | | |

*5AmMC6 indicates the 5′ Amino Modifier C6 and is not included in as a part of the identified sequence in the sequence listing. The target sequences are underlined.
**The Kcleave assay using the mismatched position 5 dsDNA target was run at 37° C.

### Determining Cleavage-Competent Fractions for RNPs

Cleavage reactions were prepared with final RNP concentrations of 100 nM and final target concentration of 100 nM. Reactions were carried out at 37° C. and initiated by the addition of the dye-labeled dsDNA target. Aliquots were taken at 5, 30, and 60 minutes and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software.

### Kcleave Assay:

Cleavage reactions were set up with a final RNP concentration of 200 nM and a final target concentration of 10 nM. Reactions were carried out at 16° C., except where otherwise noted, and initiated by the addition of the target DNA. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX: sgRNA combination replicate individually.

To test the relative specificities of engineered proteins in vitro, apparent cleavage rate constants were compared for targets with mismatched bases at various positions (5, 10, and 15 nt downstream of PAM, Table 35). Cleavage assays were performed in large excess of RNP (200 nM RNP and 1 nM target dsDNA) at 16° C., with the exception of assays measuring cleavage of the target with a mismatch at 5 nt, which were conducted at 37° C. in order to observe measurable cleavage rates. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX: sgRNA combination replicate individually.

### Results:

### Determining Cleavage-Competent Fractions for Protein Variants Compared to Reference CasX 119

To determine the cleavage-competent fraction for the tested CasX proteins, it was assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. Thus, the active (competent) fraction for each RNP was derived from the cleaved fraction over the total signal at the 60-minute timepoint, upon confirming an increase in cleaved fraction from the 5-minute timepoint, and relative plateau in cleaved fraction from the 30-minute timepoint.

Apparent competent fractions were determined for the RNPs with various CasX proteins, and are provided in Table 36.

TABLE 36
Protein variant RNP comparison of fraction competence and Kcleave rates

| | Fraction | Kcleave (sec⁻¹) | | | |
|---|---|---|---|---|---|
| | competence * | On- | Position 15 | Position 10 | Position 5 |
| RNP | (mean ± | target | mismatch | mismatch | mismatch |
| (CasX.Scaffold.Spacer) | stdev) | spacer | spacer | spacer | spacer** |
| 119.174.7.9 | 26.71 ± | 0.0292 | 0.0158 | 0.0047 | 0.0127 |
| | 9.65% | | | | |
| 491.174.7.9 | 49.23 ± | 0.0966 | 0.0552 | 0.03 | 0.0616 |
| | 6.37% | | | | |
| 515.174.7.9 | 30.15 ± | 0.0782 | 0.0625 | 0.0324 | 0.0392 |
| | 3.58% | | | | |
| 812.174.7.9 | 53.28 ± | 0.0909 | 0.0512 | 0.0142 | 0.01 |
| | 5.14% | | | | |
| 119.174.7.37 | 29.01 ± | 0.0022 | 0.0061 | 0.0028 | 0.0034 |
| | 2.67% | | | | |
| 491.174.7.37 | 48.73 ± | 0.0425 | 0.015 | 0.0297 | 0.0301 |
| | 5.44% | | | | |
| 515.174.7.37 | 43.54 ± | 0.0396 | 0.0127 | 0.0297 | 0.0347 |
| | 16.19% | | | | |
| 812.174.7.37 | 38.23 ± | 0.027 | 0.0111 | 0.0119 | 0.014 |
| | 2.49% | | | | |

* Active fraction was calculated by averaging three experimental replicates.
* The Kcleave assay using the mismatched position 5 dsDNA target was run at 37° C.

For protein variant comparison, the following CasX proteins were used with guide scaffold 316 and spacer 7.9 or guide 316 and spacer 7.37; CasX 119, CasX 491, CasX 515, and CasX 812. CasX 119 had the lowest active fraction for both spacers, indicating that CasX 491, CasX 515, and CasX 812 form more active and stable RNP with the identical guides under the tested conditions as compared to CasX 119. CasX proteins 491, 515, and 812 did not show consistent trends in their competent fractions across the two spacers, consistent with the expectation that the additional engineering following CasX 491 primarily affects target engagement and cleavage, rather than guide binding or stability.

Kcleave Assay to Understand Specificity of RNPs Formed from Protein Variants

Assays were performed to measure the apparent first-order rate constant of non-target strand cleavage (k_{cleave}), and the results are presented in Table 36, above. A drastic effect on the kinetics of CasX 812 RNP cleavage was observed for on-target versus the mismatched dsDNA target for both spacers. CasX 812 had comparable on-target cleavage rates to CasX 491 and CasX 515 for both spacers, with a slightly higher cleavage rate than 515 on spacer 7.9, which might be explained by the lower competent fraction observed for the 515 RNP with that spacer, and a lower cleavage rate on 7.37.

The off-target rates for CasX 812 were much more substantially reduced for most of the mismatched substrates. The difference in kcleave rates was readily apparent for the target with a mismatch at position 10, with 812 having a roughly 6-fold (7.9) and 2-fold (7.37) reduction in cleavage rate, as compared to its on-target rate. CasX 515, by comparison, exhibited a 2.4-fold and a 25% reduction on the same targets. A substantial difference was also observed for the position 5 mismatch targets. Even though the assay was run at 37° C. to enable measurable cleavage rates, as the position 5 mismatch targets were essentially uncleaved by the CasX RNPs at the lower temperature used for the other targets, CasX 812 against spacer 7.9 exhibited a 9-fold reduction in cleavage rate from on-target rate run at 16° C. and a 2-fold reduction for the 7.37 spacer with a position 5 mismatch. CasX 515 showed a 2-fold reduction for mismatched 7.9 and a nearly equivalent cleavage rate for 7.37 with the position 5 mismatch (note that the “equivalent” cleavage rate is due to the increased temperature).

For the position 15 mismatch substrate, CasX 812 exhibited modest reductions in cleavage rates relative to on-target rates, comparable to the reduction observed for 515. This suggests that the increased sensitivity of CasX 812 to mismatches declines by the PAM distal region, at least for the specific mismatches and spacers tested here. The increased sensitivity at positions 5 and 10 in particular correlates with the position of the G329K mutation present in CasX 812. This mutation introduces a positive charge near the RNA spacer around position 8 and may help CasX to better read out distortions caused by mismatches. Mismatches closer to this new site of contact would be more likely to significantly disrupt either R-loop propagation or allosteric activation of the RuvC (depending on the precise mechanism of increased specificity), while mismatches farther away (as in the position 15 mismatch) might have more variable effects depending on the nature of the mismatch and its effects on the broader heteroduplex structure. Taken together, these data confirm that CasX 812 is inherently more sensitive to mismatches between the RNA spacer and the DNA target and is not simply a less active enzyme, as the decrease in cleavage rate at mismatched targets is in excess of the decrease in cleavage rate at properly matched targets. This is consistent with the results presented in Examples B and C that indicate that CasX 812 is a highly specific enzyme, with lower off-target editing compared to the other nucleases tested.

### Determining Cleavage-Competent Fractions for Single Guide Variants Relative to Reference Single Guide 2

RNPs were complexed using the aforementioned methods. To isolate the effect of sgRNA identity on RNP formation, guide-limiting conditions were employed. sgRNAs with scaffolds 2, 174, 235, or 316 with spacers 7.9 or 7.37 were mixed with CasX 515 at final concentrations of 1 μM for the guide and 1.2 μM for the protein. Fraction competence was calculated as described above, and the results are provided in Table 37.

TABLE 37
Guide variant RNP comparison of fraction
competence and Kcleave assay

| RNP construct | Competent fraction | Kcleave |
|---|---|---|
| (CasX.Scaffold.Spacer) | (mean ± stdev) | (sec⁻¹) |
| 515.2.7.9 | 28.146.87% | 0.1346 ± 0.0118 |
| 515.174.7.9 | 42.779.62% | 0.1723 ± 0.0046 |
| 515.235.7.9 | 34.111.15% | 0.1696 ± 0.0571 |
| 515.316.7.9 | 30.896.87% | 0.1413 ± 0.0301 |
| 515.2.7.37 | 10.422.24% | 0.0204 ± 0.0002 |
| 515.174.7.37 | 19.962.88% | 0.0534 ± 0.0200 |
| 515.235.7.37 | 32.6411.60% | 0.0647 ± 0.0163 |
| 515.316.7.37 | 26.9811.08% | 0.0851 ± 0.0071 |

* active fraction was calculated by averaging two experimental replicates

Given the complex folding structure of the CasX guide, fraction competence is expected to largely be determined by how much of the guide is properly folded for interaction with the protein. All guides with engineered scaffolds showed improvements over scaffold 2, but guides with scaffold 235 or 316 showed improvements relative to 174 for spacer 7.37. This is consistent with the introduction of mutations in the pseudoknot and triplex that are expected to stabilize the properly folded form.

Higher competent fractions of all guides were observed for spacer 7.9. For this spacer, scaffold 174 had the highest competent fraction, followed by scaffolds 316, 235, and 2. Proper guide folding is expected to be highly dependent on the potential for undesired interactions between the scaffold and spacer sequences, so the observed differences may be attributable to differential sequence-specific interactions, variations in prep quality, or noise in the assay.

Determining k_{cleave} for Single Guide Variants Compared to Reference Scaffold 2

Cleavage assays were performed with CasX 515 and guides with reference scaffold 2 compared to guides with scaffolds 174, 235, or 316 with spacer 7.9 or 7.37 to determine relative cleavage rates. The mean and standard deviation of three replicates with independent fits are presented in Table 37, above.

To reduce cleavage kinetics to a range measurable with the assay, the cleavage reactions were incubated at 16° C. Under these conditions, all guides supported faster cleavage rates as compared to scaffold 2. For spacer 7.37, the cleavage kinetics aligned with those guides that contributed to the highest fraction competence, with the highest cleavage rate being sg174 (0.1723 s⁻¹), followed by scaffold 235 (0.1696 s⁻¹) and scaffold 316 (0.1413 s⁻¹), versus scaffold 2 (0.1346 s⁻¹). For spacer 7.9, scaffold 316 yielded the highest cleavage rate (0.0851 s⁻¹), followed by scaffold 235 (0.0647 s⁻¹) and sg174 (0.0534 s⁻¹), versus scaffold 2 (0.0204 s⁻¹). The fraction competence and kcleave data did not demonstrate differences across the engineered variants that were consistent across both spacers, although all are consistently better than scaffold 2. This suggests that the improvements seen for scaffold 235 and 316 over 174 are primarily due to behavior in the cell, whether it be stability in the cytoplasm, folding in the cytoplasm, transcription when delivered via plasmid or AAV, or refolding ability when delivered via LNP, that are not captured by guides that have been in vitro transcribed, refolded, and tested for cleavage biochemically.

### Example 9: Suppression of Apo (a) Secretion in Primary Human Hepatocytes

Experiments were conducted to demonstrate the ability of a CasX:gRNA system to reduce apo (a) secretion in primary human hepatocytes.

### Materials and Methods

In Vitro Delivery of gRNA and CasX mRNA Via Lipofection:

Editing at the LPA locus and consequential effects on secreted apo (a) levels were assessed using CasX 515 mRNA (SEQ ID NO: 19755) co-delivered with gRNA comprising scaffold 316 (SEQ ID NO: 466) and LPA-targeting sequences (see Table 38) that, in each case, were converted into the chemically-modified v1 versions (detailed in Example 7) for delivery, into primary human hepatocytes by lipofection. As experimental control, CasX 515 mRNA was co-delivered with a non-targeting gRNA.

TABLE 38
LPA-targeting targeting sequences used in this
example.

| Targeting | | SEQ ID |
|---|---|---|
| sequence ID | Targeting sequence (RNA) | NO: |
| TG-48-239 | UGUGACAGUGGUGGAGGAUA | 820 |
| TG-48-351 | CAAUAACAAGGAGCUGGGCU | 901 |

### Assessment of Apo (a) Secretion by Western Blot Using an Automated Capillary Separation and Detection System, Jess (Bio-Techne):

Briefly, primary human hepatocytes were lipofected with an in vitro transcribed mRNA coding for CasX 515 and an LPA-targeting gRNA using lipofectamine. Six days after lipofection, and 72 hours after a media change, supernatant was harvested and the apo (a) secreted into the media was measured. Total protein was quantified and diluted to 2 ug/μL prior to Western blotting, and secreted apo (a) was detected using a monoclonal apo (a) antibody (Abcam: Ab208184). The amount of protein was determined using Compass software to calculate the area under the curve (AUC) for the apo (a) peak detected at ˜450 kDa. AUC values were compared to a standard curve of fixed apo (a) concentrations to quantify final protein concentrations. While a standard curve was not included for every Jess run, 2 or more calibrator samples were included across multiple Jess runs to 1) calculate protein concentrations based on a previous standard curve and 2) evaluate protein concentrations from multiple Jess runs.

### Results

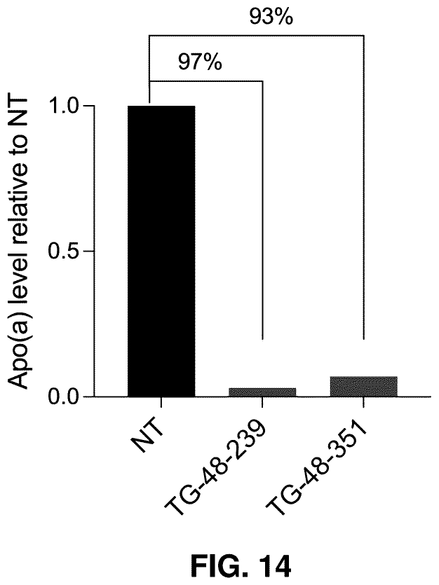

The ability of CasX 515 and gRNA comprising scaffold 235 (SEQ ID NO: 19713) and an LPA-targeting sequence to reduce secretion of apo (a) was assessed. FIG. 14 and Table 39, below, provide the relative level of apo (a) produced by primary human hepatocytes with each individual targeting sequence, rounded to the nearest hundredth.

TABLE 39
Apo(a) levels relative to non-targeting
gRNA in primary human hepatocytes

| Targeting sequence ID | Apo(a) level | Percent Reduction |
|---|---|---|
| TG-48-239 | 0.03 | 97% |
| TG-48-351 | 0.07 | 93% |

The results demonstrate that CasX 515 and an LPA-targeting gRNA were able to edit the LPA locus in primary human hepatocytes with gRNAs, markedly reducing apo (a) secretion.

### Example 10: Assessment of CasX Variant 515 and gRNAs Using Scaffold Variant 235 with LPA-Targeting Targeting Sequences to Induce Off-Target Editing

Experiments were performed to assess the potential off-target editing of various CasX proteins and gRNAs with targeting sequences designed to target the human LPA locus. Here, in silico analysis was performed to determine and assess the predicted off-target sites for the various LPA-targeting targeting sequences. Furthermore, cut site incorporation and sequencing (CSI-seq) was performed in cells, experimentally assessing the off-target activity of gRNAs containing human LPA-targeting targeting sequences in complex with CasX variant 515.

### Materials and Methods

### CSI-Seq Assay:

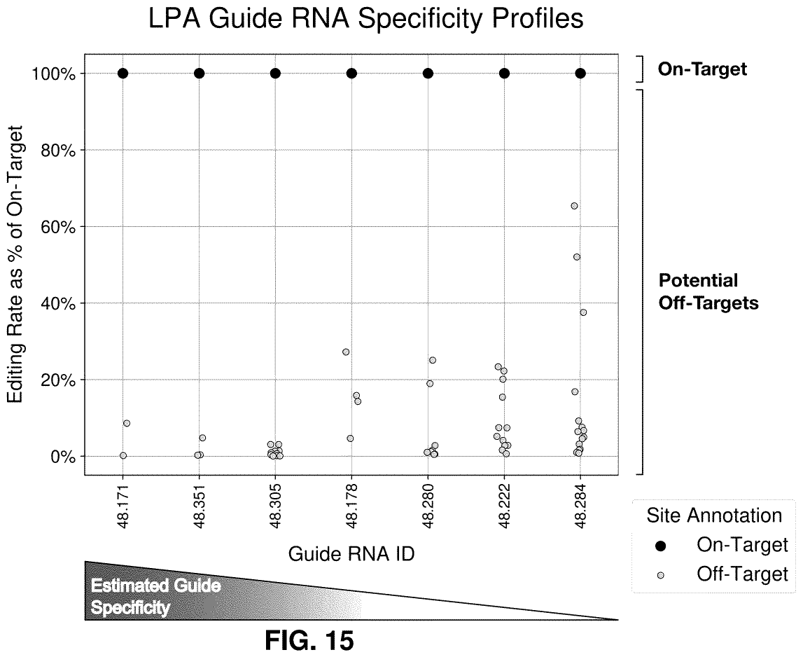

˜700,000 HEK293 cells were nucleofected using a Lonza nucleofector with 500 ng of plasmid DNA encoding CasX 515, gRNA with scaffold 235 and LPA-targeting targeting sequences (sequences listed in Table 40), and 100 pmol of a double stranded DNA fragment to be incorporated at cut-sites. Nucleofected cells were then plated onto a 6-well plate and grown for five days, after which they were harvested for genomic DNA (gDNA) extraction using a Zymo QuickDNA™ miniprep kit. Samples were normalized and sequenced on an Illumina NextSeq™. Subsequently, samples were analyzed to identify off-target editing events. Briefly, reads were filtered to eliminate off-target priming sites from analysis. These filtered reads are referred to herein as “CSI-seq reads.” Genomic sites with multiple mapped reads were required to have aligned reads in both orientations to further reduce false positives. Sites with a nearby sequence containing seven or fewer mismatches or RNA/DNA bulges away from the search targeting sequence were identified as potential off-target sites. The number of CSI-seq reads at each site relative to the number of CSI-seq reads at the on-target site was expected to be roughly proportional to the relative amount of off-target editing at that site. FIG. 15 presents the level of off-target editing of the tested targeting sequences at the given off-target sites normalized to the level of on-target editing for each targeting sequence, calculated as the number of off-target CSI-seq reads for a targeting sequence at a particular site divided by the number of on-target CSI-reads for the targeting sequence, multiplied by 100.

### Results

Potential off-target sites were identified using a CSI-seq assay for each LPA-targeting sequence. The results are depicted as the number of off-target CSI-seq reads at a particular off-target site, normalized to the number of on-target CSI-seq reads obtained for that targeting sequence, as described above. Targeting sequences with few off-target sites and relatively few CSI-seq reads at each site were considered to have minimal off-target risk, while targeting sequences with many off-target sites with moderate numbers of CSI-seq reads or a top off-target site with a high number of reads were considered to have a high off-target risk.

While several targeting sequences show minimal off-target activity, others had a top off-target site with reads >25% of on-target, with one with a top off-target with 65% of on-target CSI-seq reads (FIG. 15). The top off-target site was very often not the identified site with the fewest mismatches, and in many cases sites with fewer mismatches could be identified computationally but did not appear in the CSI-seq hits. This observation, consistent with published data for other CRISPR nucleases, demonstrates the importance of experimentally determining the potential off-target editing locations for targeting sequences. These results show that two targeting sequences, 48.305 and 48.351, with minimal off-target editing effects have been identified for LPA, enabling the safe and efficacious targeting of the locus.

TABLE 40
Sequences of human LPA-targeting targeting sequences tested in a CSI-seq
assay

| Targeting | | SEQ | | SEQ |
|---|---|---|---|---|
| sequence | Targeting sequence DNA | ID | Targeting sequence RNA | ID |
| ID | sequence | NO: | sequence | NO: |
| 48.171 | GTCACCAGGCATTGTGTCAG | 19806 | GUCACCAGGCAUUGUGUC | 877 |
| | | | AG | |
| 48.178 | TCAGAAGAAGCCTCTGTGCT | 19807 | UCAGAAGAAGCCUCUGUG | 878 |
| | | | CU | |
| 48.222 | TGTGATAGTGGTGGAGAGTG | 19808 | UGUGAUAGUGGUGGAGA | 817 |
| | | | GUG | |
| 48.280 | GAAGGAGGCCCTAGGCTTG | 19809 | GAAGGAGGCCCUAGGCUU | 787 |
| | G | | GG | |
| 48.284 | AGGGACAAATAAATGGGCA | 19810 | AGGGACAAAUAAAUGGGC | 826 |
| | G | | AG | |
| 48.305 | AGCAGTGAGCAGCAGTCAG | 19811 | AGCAGUGAGCAGCAGUCA | 792 |
| | C | | GC | |
| 48.351 | CAATAACAAGGAGCTGGGC | 19812 | CAAUAACAAGGAGCUGGG | 901 |
| | T | | CU | |

### Example 11: Functional Assessments of CasX: GRNA Systems Targeting the Human LPA Locus in Primary Human Hepatocytes

Experiments were performed to assess the ability of a CasX:gRNA system to reduce apo (a) secretion in primary human hepatocytes. This set of experiments was used to narrow the selection of LPA-targeting sequences from 60 to the 8 LPA-targeting sequences resulting in the greatest editing.

### Materials and Methods

In Vitro Delivery of gRNA and CasX mRNA Via Lipofection:

Editing at the LPA locus and consequential effects on secreted apo (a) levels were assessed using CasX 515 mRNA (SEQ ID NO: 19755, Table 42) co-delivered with the 316 gRNA scaffold (SEQ ID NO: 466) with LPA-targeting sequences (see Table 41) that, in each case, were converted into the chemically-modified v1 versions (detailed in Example 7) for delivery, into primary human hepatocytes by lipofection. As experimental control, CasX 515 mRNA was co-delivered with a non-targeting gRNA.

### Assessment of Apo (a) Secretion by Western Blot Using an Automated Capillary Separation and Detection System, Jess (Bio-Techne):

Briefly, primary human hepatocytes were lipofected with an in vitro transcribed mRNA coding for CasX 515 and an LPA-targeting gRNA using lipofectamine. Six days after lipofection, and 72 hours after a media change, supernatant was harvested and the apo (a) secreted into the media was measured. Total protein was quantified and diluted to 2 ug/μL prior to Western blotting, and secreted apo (a) was detected using a monoclonal apo (a) antibody (Abcam: Ab208184). The amount of protein was determined using Compass software to calculate the area under the curve (AUC) for the apo (a) peak detected at ˜450 kDa. AUC values were compared to a standard curve of fixed apo (a) concentrations to quantify final protein concentrations. While a standard curve was not included for every Jess run, 2 or more calibrator samples were included across multiple Jess runs to 1) calculate protein concentrations based on a previous standard curve and 2) evaluate protein concentrations from multiple Jess runs. mRNA level measurement by qPCR:

Measurement of LPA mRNA expression was performed using a single-step RT-qPCR approach, 25-50 uL of sample lysate was used for RNA extraction. GAPDH primers/probe were used for normalizing transcript levels (IDT; Hs.PT.39a.22214836). Percent change in RNA was calculated by comparing to samples treated with a non-targeting gRNA (Table 43).

### Assessment of Editing of LPA Gene by Next-Generation Sequencing (NGS):

Genomic DNA was amplified via PCR with primers specific to the target genomic location of interest to form a target amplicon. These primers contained additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 program. The percentage of reads modified in a window around the 3′ end of the targeting sequence was determined. The editing activity of the CasX molecule was quantified as the total percent of reads that contained deletions anywhere within this window (Table 43). Each LPA-targeting sequence was assessed in 1-3 independent replicates. Table 43, below, provides the mean deletion rate for the tested LPA-targeting sequences.

### Results

The ability of CasX 515 and gRNA comprising scaffold 316 and an LPA-targeting sequence to reduce secretion of apo (a) was assessed. Table 43, below, provides the relative level of apo (a) produced by primary human hepatocytes with each individual targeting sequence, rounded to the nearest hundredth, the percent change in mRNA by qPCR, and the editing rate measured by NGS as percent indel. These results were used to select the top ten LPA-targeting sequences, based on combined editing rate, mRNA reduction, and protein reduction. Three LPA-targeting sequences (TG-48-280, TG-48-223, TG-48-239) were excluded from this set due to cutting the target gene in multiple locations (data not shown), resulting in the selection of eight LPA-targeting sequences for further evaluation (Table 44).

The results demonstrate that CasX 515 and an LPA-targeting gRNA were able to edit the LPA locus, reduce mRNA, and reduce apo (a) secretion in primary human hepatocytes.

TABLE 41
LPA-targeting targeting sequences used in this
example.

| Targeting | | SEQ |
|---|---|---|
| sequence ID | Targeting sequence (RNA) | ID NO: |
| TG-48-107 | UCUGCCUUUCUACUGGAUAC | 776 |
| TG-48-178 | UCAGAAGAAGCCUCUGUGCU | 878 |
| TG-48-222 | UGUGAUAGUGGUGGAGAGUG | 817 |
| TG-48-284 | AGGGACAAAUAAAUGGGCAG | 826 |
| TG-48-303 | UUCUAGGUUUGGAAAGCACU | 896 |
| TG-48-305 | AGCAGUGAGCAGCAGUCAGC | 792 |
| TG-48-317 | UGGAGCCCACACAAGCAGAU | 898 |
| TG-48-351 | CAAUAACAAGGAGCUGGGCU | 901 |
| TG-48-005 | AUGGAAGCUUGGCUAUCUUC | 811 |
| TG-48-007 | CUAGAAGUCUCGCUGCUUUC | 809 |
| TG-48-009 | UAUCCCCUGCACUCCCUACA | 771 |
| TG-48-015 | AGAGAACGGUGCUCCCUCAC | 852 |
| TG-48-016 | CUAGAAUCUGCUAUUUGAGC | 851 |
| TG-48-017 | UGGCAACUUGCGGUAGGUUU | 799 |
| TG-48-056 | GACUACCAAGCAUCGGGCAG | 853 |
| TG-48-083 | CAGUUCAAAAUCUUGUAUAU | 805 |
| TG-48-087 | AGCACCGUGACAGUCUUCAC | 806 |
| TG-48-088 | GCCCAUUCAUUCCAGCACCG | 854 |
| TG-48-102 | AGCAUAGAGCAUAUUAAAUU | 807 |
| TG-48-104 | UGCAAAGUCCUUGGCCACAG | 775 |
| TG-48-109 | ACUGGAUACUUGUCAUCGGC | 857 |
| TG-48-111 | CUGCAGUGUGUCAUCUUUCA | 858 |
| TG-48-113 | AGGGAAUACAGGGUCAGGGG | 859 |
| TG-48-118 | UCUGUUUUAAUUUUAGCAGC | 862 |
| TG-48-119 | GUUUUAAUUUUAGCAGCACC | 863 |
| TG-48-124 | GUGGUCCUAUUAUGUUGAUG | 864 |
| TG-48-127 | GCCAUGUUGGGGGAAGCCUC | 865 |
| TG-48-137 | UUUCUUUUCAUUUCAGCACC | 869 |
| TG-48-139 | GUGGUAGCACUCCUGCACCC | 813 |
| TG-48-141 | UGUGACAGUAAUGAAGUAUG | 814 |
| TG-48-142 | UCCUGUGACAGUAAUGAAGU | 871 |
| TG-48-145 | GCAGUAGUUCUUGAUCAAGC | 840 |
| TG-48-151 | GCAUCUGAGCAUCGUGUCAG | 873 |
| TG-48-162 | GUUUUAAUUUCAGCACUGAC | 875 |
| TG-48-169 | GGGGUCCGACUAUGCUGGUG | 876 |
| TG-48-171 | GUCACCAGGCAUUGUGUCAG | 877 |
| TG-48-179 | UACCUUCUUCAGAAGAAGCC | 815 |
| TG-48-193 | GAAACAGCCGUGGACGUCGC | 780 |
| TG-48-196 | CUGAUGCCAGUGUGGUGUCA | 881 |
| TG-48-199 | CUUGGGAGAAAACUCAAAGA | 883 |
| TG-48-223 | UCCUGUGAUAGUGGUGGAGA | 884 |
| TG-48-229 | UUUUAUGGCUAACAUGAUAG | 818 |
| TG-48-235 | AAGCACAGAGGCUCCUUCUG | 819 |
| TG-48-239 | UGUGACAGUGGUGGAGGAUA | 820 |
| TG-48-267 | UGCAGUAGUUCUCGGUCAGG | 824 |
| TG-48-280 | GAAGGAGGCCCUAGGCUUGG | 787 |
| TG-48-281 | AAGCCUAGGGCCUCCUUCUG | 825 |
| TG-48-283 | UUCCAGGGACAAAUAAAUGG | 788 |
| TG-48-286 | UUGUGUAGCACCAGGGACCA | 789 |
| TG-48-288 | UGGAUUCAUUGUGUAGCACC | 890 |
| TG-48-299 | UCAUUGCUCUGAGACUCUUA | 828 |
| TG-48-300 | CCUUCCCCUGAUCCAAGCCU | 829 |
| TG-48-306 | UCAAGCAGUGAGCAGCAGUC | 897 |
| TG-48-324 | GUCCUGGCGGUGACCAUGUA | 793 |
| TG-48-336 | CUCUUAUCAGGAUGUUUGCC | 833 |
| TG-48-341 | GCAGAGCUGCAUGGAGGAAG | 834 |
| TG-48-342 | UCCAUGCAGCUCUGCGGAAG | 835 |
| TG-48-343 | GUGUCAGAAAACUGACACCG | 843 |
| TG-48-358 | CCUACCUCUGUUUAGGGUGA | 836 |
| TG-48-360 | CUCUGUCUCCCGUCCAAUUA | 795 |

TABLE 42
Full-length protein sequence of CasX 515 molecule with NLS.

| | | SEQ ID |
|---|---|---|
| CasX | AA sequence | NO |
| CasX 515 | MAPAAKRVKLDSRQEIKRINKIRRRLVKDSNTKKAGKTGPMKTLLVRVMT | 19757 |
| with | PDLRERLENLRKKPENIPQPISNTSRANLNKLLTDYTEMKKAILHVYWEEF | |
| flanking | QKDPVGLMSRVAQPASKKIDQNKLKPEMDEKGNLTTAGFACSQCGQPLFV | |
| c-MYC | YKLEQVSEKGKAYTNYFGRCNVAEHEKLILLAQLKPEKDSDEAVTYSLGK | |
| NLSes | FGQRALDFYSIHVTKESTHPVKPLAQIAGNRYASGPVGKALSDACMGTIAS | |
| | FLSKYQDIIIEHQKVVKGNQKRLESLRELAGKENLEYPSVTLPPQPHTKEGV | |
| | DAYNEVIARVRMWVNLNLWQKLKLSRDDAKPLLRLKGFPSFPLVERQAN | |
| | EVDWWDMVCNVKKLINEKKEDGKVFWQNLAGYKRQEALRPYLSSEEDR | |
| | KKGKKFARYQLGDLLLHLEKKHGEDWGKVYDEAWERIDKKVEGLSKHIK | |
| | LEEERRSEDAQSKAALTDWLRAKASFVIEGLKEADKDEFCRCELKLQKWY | |
| | GDLRGKPFAIEAENSILDISGFSKQYNCAFIWQKDGVKKLNLYLIINYFKGG | |
| | KLRFKKIKPEAFEANRFYTVINKKSGEIVPMEVNFNFDDPNLIILPLAFGKRQ | |
| | GREFIWNDLLSLETGSLKLANGRVIEKTLYNRRTRQDEPALFVALTFERRE | |
| | VLDSSNIKPMNLIGVDRGENIPAVIALTDPEGCPLSRFKDSLGNPTHILRIGES | |
| | YKEKQRTIQAKKEVEQRRAGGYSRKYASKAKNLADDMVRNTARDLLYY | |
| | AVTQDAMLIFENLSRGFGRQGKRTFMAERQYTRMEDWLTAKLAYEGLPS | |
| | KTYLSKTLAQYTSKTCSNCGFTITSADYDRVLEKLKKTATGWMTTINGKEL | |
| | KVEGQITYYNRYKRQNVVKDLSVELDRLSEESVNNDISSWTKGRSGEALSL | |
| | LKKRFSHRPVQEKFVCLNCGFETHADEQAALNIARSWLFLRSQEYKKYQT | |
| | NKTTGNTDKRAFVETWQSFYRKKLKEVWKPAVGSPAAKRVKLD | |

TABLE 43
Apo(a) levels relative to non-targeting
gRNA in primary human hepatocytes

| | Jess % KD | qPCR % KD | % Indel Adjusted |
|---|---|---|---|
| Targeting sequence ID | (average) | (average) | (Max) |
| TG-48-005 | −13.08 | 6.85 | 0 |
| TG-48-007 | −35.25 | −13.49 | 77.702 |
| TG-48-009 | −36.66 | −12 | 55.442 |
| TG-48-015 | −15.53 | −23.6 | 84.959 |
| TG-48-016 | −22.43 | 10.7 | 63.353 |
| TG-48-017 | −32.23 | −15.72 | 81.131 |
| TG-48-056 | −25.38 | −11.28 | 0 |
| TG-48-083 | −40.17 | −13.27 | 76.422 |
| TG-48-087 | −13.71 | −16.78 | 0 |
| TG-48-088 | −34.15 | −34.43 | 0 |
| TG-48-102 | −29.18 | −18.31 | 37.547 |
| TG-48-104 | −18.48 | −26.3 | 39.935 |
| TG-48-107 | −46.37 | −22.3 | 77.172 |
| TG-48-109 | −34.45 | −18.54 | 48.609 |
| TG-48-111 | −4.66 | 19.2 | 56.251 |
| TG-48-113 | −21.4 | NA | 70.91 |
| TG-48-118 | −7.49 | 7.43 | 2.548 |
| TG-48-119 | 14.72 | −12.09 | 0.742 |
| TG-48-124 | −16.73 | 18.04 | 34.449 |
| TG-48-127 | 0.89 | −3.37 | 0 |
| TG-48-137 | 22.08 | 4.92 | 0.367 |
| TG-48-139 | 3.95 | −13.57 | 41.115 |
| TG-48-141 | −7.48 | −27.86 | 50.719 |
| TG-48-142 | −14.04 | −16.71 | 47.697 |
| TG-48-145 | −9.39 | −8.69 | 22.584 |
| TG-48-151 | 1.29 | −1.81 | 23.147 |
| TG-48-162 | 11.67 | −1.45 | 1.746 |
| TG-48-169 | −27.44 | NA | 35.291 |
| TG-48-171 | −19.71 | −6.65 | 20.113 |
| TG-48-178 | −43.2 | −16.52 | 55.591 |
| TG-48-179 | 4.62 | 8.81 | 32.168 |
| TG-48-193 | −5.54 | 1.18 | 19.604 |
| TG-48-196 | −8.9 | −14.98 | 0.397 |
| TG-48-199 | −12.96 | −36.22 | 43.481 |
| TG-48-222 | −72.66 | −33.87 | 71.82 |
| TG-48-223 | −74.07 | −37.61 | 59.204 |
| TG-48-229 | −11.43 | −9.4 | 29.387 |
| TG-48-235 | −11.05 | −21.96 | 27.207 |
| TG-48-239 | −86.37 | −32.34 | 23.567 |
| TG-48-267 | −5.02 | 14.91 | 0.794 |
| TG-48-280 | −53.24 | −5.95 | 67.553 |
| TG-48-281 | −8.65 | −1.93 | 9.604 |
| TG-48-283 | −36.6 | −11.71 | 27.07 |
| TG-48-284 | −80.34 | −44.9 | 69.564 |
| TG-48-286 | −28.79 | −19.42 | 43.675 |
| TG-48-288 | −35.22 | −14.82 | 23.7 |
| TG-48-299 | −11.38 | −7.09 | 15.948 |
| TG-48-300 | −5.86 | 16.3 | 42.249 |
| TG-48-303 | −66.92 | −10.32 | 66.48 |
| TG-48-305 | −70.13 | −19.84 | 63.031 |
| TG-48-306 | −21.44 | −3.72 | 36.276 |
| TG-48-317 | −78.83 | −19.47 | 76.382 |
| TG-48-324 | −27.78 | −2.5 | 38.185 |
| TG-48-336 | −26.62 | −31.17 | 79.414 |
| TG-48-341 | −20.88 | −16.87 | 80.807 |
| TG-48-342 | −25.89 | −15.4 | 36.205 |
| TG-48-343 | −9.99 | −2.21 | 36.766 |
| TG-48-351 | −79.47 | −11.22 | 77.612 |
| TG-48-358 | −33.46 | −28.15 | 41.522 |
| TG-48-360 | −43.88 | NA | 41.39 |

TABLE 44
LPA-Targeting sequences for further evaluation

| Targeting | | SEQ |
|---|---|---|
| sequence ID | Targeting sequence (RNA) | ID NO: |
| TG-48-107 | UCUGCCUUUCUACUGGAUAC | 776 |
| TG-48-178 | UCAGAAGAAGCCUCUGUGCU | 878 |
| TG-48-222 | UGUGAUAGUGGUGGAGAGUG | 817 |
| TG-48-284 | AGGGACAAAUAAAUGGGCAG | 826 |
| TG-48-303 | UUCUAGGUUUGGAAAGCACU | 896 |
| TG-48-305 | AGCAGUGAGCAGCAGUCAGC | 792 |
| TG-48-317 | UGGAGCCCACACAAGCAGAU | 898 |
| TG-48-351 | CAAUAACAAGGAGCUGGGCU | 901 |

### Example 12: Functional Assessments of CasX: GRNA Systems Targeting the Human LPA Locus

Experiments were performed to demonstrate that delivery of LNPs encapsulating CasX mRNA and an LPA-targeting gRNA induces editing at the endogenous human LPA locus in primary human hepatocytes (PHH) and thus, induce a reduction of secreted apo (a). CasX 515 (SEQ ID NO: 19755) and guide scaffold 316 (SEQ ID NO: 466) were selected for assessment in this Example.

### Materials and Methods:

Apo (a) knockdown in primary human hepatocytes, as demonstrated in Example 9, provided an experimental basis for lipofection of eight top editing targeting sequences (Table 48) into PHH cells for assessment of apo (a) secretion reduction. mRNA production:

Generation of CasX mRNAs encoding CasX 515 was performed by IVT, as disclosed in Example 5. Briefly, constructs encoding a synthetic 5′UTR, a codon-optimized CasX 515 with flanking c-MYC NLSes, and a 3′UTR derived from the mouse hemoglobin alpha (mHBA) were cloned into a plasmid comprising a T7 promoter and 79-nucleotide poly (A) tail. The resulting plasmid was linearized prior to use for IVT reactions, which were carried out with CleanCap® AG and N1-methyl-pseudouridine (as described in Example 5). The DNA sequence encoding the CasX 515 mRNA molecule is provided in Table 45, and the corresponding mRNA sequence showing the chemical modifications is provided in Table 46. The protein sequence for CasX 515 resulting from expression of the IVT mRNA molecule is provided in Table 47.

Synthesis of gRNAs:

gRNAs targeting the human LPA locus were chemically synthesized by methods known in the art) that, in each case, were converted into the chemically-modified v1 versions (detailed in Example 7) for delivery. The sequences of the 8 LPA-targeting sequences are listed in Table 44 in Example 11.

### Formulation of Lipid Nanoparticles (LNPs):

CasX 515 mRNA and targeting gRNAs were encapsulated into LNPs made from Ionizable lipid mix containing ALC0315 Ionizable lipid: 18:0 PC (DSPC):Cholesterol:DMG-PEG2000 using an in-house custom-made T-mixer at N/P 6.

Briefly, to formulate LNPs, as a coformulation (containing both mRNA and sgRNA in a single formulation) or split formulations (containing either CasX mRNA or only sgRNA) were diluted at a fixed ratio for coformulation or separately for split formulation in a 25 mM sodium acetate buffer, pH 4.0. ALC315 Ionizable lipid mix at a molar ratio of 50:10:38.5:1.5% of above-mentioned lipids using anhydrous ethanol. The RNA and lipid phases were run through a custom-made T-mixer at a flow rate ratio of 3:1 and a flow rate of 20 mL/min. After formulation, the LNPs were transferred and dialyzed using a 10 KDa membrane dialysis cassette (Thermo Scientific™) and buffer exchanged into 1×PBS to decrease the ethanol concentration and increase the pH to 7.4, leading to formation of a mature and stabilized particles. Following dialysis, the RNA-LNPs were buffer exchanged into 300 mM sucrose in PBS at pH 7.4 storage buffer and concentrated to appropriate concentration using 100 kDa Amicon®-Ultra Centrifugal Filters (Millipore) and sterile-filtered. Formulated LNPs were then subjected to one free-thaw cycle at −80° C. and analyzed on a Stunner (Unchained Labs) to determine their average particle size (d. nm.) and polydispersity index (PDI). Encapsulation efficiency and RNA concentration was determined by RiboGreen™ assay using Invitrogen's Quant-iT™ Ribogreen™ RNA assay kit. LNPs were used in various experiments as described herein to deliver CasX mRNA and gRNA to target cells and tissue by mixing a 1:1 mass ratio of mRNA containing LNP and gRNA containing LNP.
Delivery of LNPs Encapsulating CasX mRNA and Targeting gRNA into Primary Human Hepatocytes:

A single lot (lot #271) of primary human hepatocytes (Lonza Biologics), was used in these experiments to assess CasX:gRNA-mediated editing at the human LPA locus when delivered by LNPs. Approximately 60,000 cells/well in a collagen coated 96-well plate, cultured in Williams' E media supplemented with FBS, PenStrep, L-glutamine, ITS (insulin, transferrin, sodium selenite), dexamethasone, and Z-VAD-FMK. The next day seeded cells were treated with LNPs, pre-incubated with 10% Human Serum overnight. These LNPs were formulated using a split formulation to encapsulate CasX 515 mRNA and an LPA-targeting gRNA at various doses (7.33e-004 to 19.5 pmol mRNA+gRNA). Media was changed 24 h after LNP treatment, and cells were cultured in a Geltrex-sandwich for five additional days prior to harvesting 1) the media supernatant to measure LPA secretion levels and 2) treated cells for gDNA extraction for editing assessment at the LPA locus by NGS. Briefly, for editing assessment, amplicons were amplified from 200 ng of extracted gDNA with primers targeting the human LPA locus and processed as described in Example 5. Treatment with LNPs encapsulating a non-targeting gRNA and CasX 515 mRNA served as an experimental control.

Primary human hepatocytes were treated with CasX 515 and an LPA-targeting gRNA comprising a targeting sequence selected from those listed in Table 44 that, in each case, were converted into the chemically-modified v1 versions (detailed in Example 7) for delivery. A non-targeting gRNA was used as a control for these experiments. Apo (a) secretion levels were measured by Western Blot using an automated capillary separation and detection system, Jess (Bio-Techne, “Jess run”).

### Western Blot by Automated Capillary Separation and Detection System, Jess (Bio-Techne):

Briefly, apo (a) secreted into conditioned media was collected 6 days after treatment, and 72 hours following a media change for a given cell line/sample. Total protein was quantified and diluted to 2 μg/uL prior to Western blotting. Apo (a) was detected using a monoclonal apo (a) antibody (Abcam: Ab208184). Compass software was used to calculate the amount of protein detected, by measuring the area under the curve (AUC) for the apo (a) peak detected at ˜450 kDa. AUC values were compared to a standard curve of fixed apo (a) concentrations to quantify final protein concentrations. While a standard curve was not included for every Jess run, 2 or more calibrator samples were included across multiple Jess runs to 1) calculate protein concentrations based on a previous standard curve and 2) evaluate protein concentrations from multiple Jess runs.

### Assessment of Editing of LPA Gene by Next-Generation Sequencing (NGS):

Genomic DNA was amplified via PCR with primers specific to the target genomic location of interest to form a target amplicon. These primers contained additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 program. The percentage of reads modified in a window around the 3′ end of the targeting sequence was determined. The editing activity of the CasX molecule was quantified as the total percent of reads that contained deletions anywhere within this window. Maximum response (Emax) and the concentration producing 50% of Emax (EC₅₀) are reported in Table 49. Each LPA-targeting sequence was assessed in 2-5 independent replicates.

### Results

The ability of CasX 515 and gRNA comprising scaffold 316 and an LPA-targeting sequence to reduce secretion of apo (a) was assessed. Table 49, below, provides the Maximum response (Emax) and the concentration producing 50% of Emax (EC₅₀) in primary human hepatocytes with each individual LPA-targeting sequence, rounded to the nearest hundredth, for protein reduction by Jess, and editing rate measured by NGS. These results were used to select the top four LPA-targeting sequences (Table 50).

The results from these experiments demonstrate that delivery of LNPs encapsulating a CasX mRNA and LPA-targeting gRNA induced editing at the endogenous human LPA locus in primary human hepatocytes that resulted in substantial reduction in secreted apo (a) levels.

TABLE 45
DNA sequence encoding the CasX 515 mRNA molecule assessed in this
example*.

| Component | | | SEQ ID |
|---|---|---|---|
| (ID) | Description | DNA sequence | NO |
| 5′UTR + | Synthetic | AAATAAGAGAGAAAAGAAGAGTAAGAAGAAATATA | 19739 |
| Kozak | | AGAGCCACC | |
| sequence | | | |
| (underlined) | | | |
| START | | ATGGCCCCTGCTGCCAAGAGAGTGAAGCTGGATAGC | 19734 |
| codon + c- | | AGA | |
| MYC NLS | | | |
| CasX 515 | | CAGGAGATCAAGCGGATTAATAAAATTCGGAGAAGA | 19751 |
| | | CTGGTGAAGGATTCTAACACAAAGAAGGCTGGCAAG | |
| | | ACAGGCCCTATGAAGACACTGCTGGTGAGAGTGATG | |
| | | ACACCCGACCTGAGAGAAAGACTGGAAAACCTGAGA | |
| | | AAGAAGCCTGAGAATATCCCCCAGCCCATCAGCAAC | |
| | | ACAAGCCGGGCCAACCTGAATAAGCTGCTGACCGAC | |
| | | TACACCGAAATGAAGAAGGCCATCCTGCACGTGTATT | |
| | | GGGAAGAGTTCCAGAAAGACCCAGTCGGCCTGATGA | |
| | | GCAGAGTGGCTCAGCCTGCCAGCAAGAAGATCGATC | |
| | | AGAACAAGCTGAAGCCCGAAATGGACGAGAAGGGGA | |
| | | ACCTGACAACCGCCGGCTTTGCCTGTAGCCAGTGCGG | |
| | | CCAGCCCCTGTTTGTGTACAAACTGGAACAGGTGAGC | |
| | | GAAAAGGGCAAGGCTTACACGAATTACTTCGGCAGA | |
| | | TGCAACGTGGCCGAGCACGAGAAGCTGATCCTGCTG | |
| | | GCCCAGCTGAAGCCTGAGAAGGATAGCGATGAGGCA | |
| | | GTGACATATTCCCTGGGCAAGTTCGGACAGCGGGCCC | |
| | | TGGATTTTTATTCCATTCATGTGACCAAGGAATCCAC | |
| | | CCACCCCGTCAAGCCTCTTGCCCAAATTGCCGGCAAC | |
| | | AGATACGCCAGCGGCCCCGTGGGCAAGGCCCTGAGC | |
| | | GACGCCTGTATGGGCACCATCGCCAGCTTCCTGTCTA | |
| | | AGTACCAGGACATTATCATCGAGCACCAGAAGGTGG | |
| | | TGAAGGGCAACCAGAAGAGACTGGAGAGCCTGCGCG | |
| | | AGCTGGCCGGCAAGGAAAACCTGGAGTATCCTAGCG | |
| | | TGACCCTGCCTCCTCAGCCTCATACAAAGGAGGGCGT | |
| | | GGATGCCTACAACGAAGTGATCGCCCGGGTGCGGAT | |
| | | GTGGGTGAACCTGAATCTGTGGCAGAAGCTGAAGCT | |
| | | GTCTAGAGACGACGCCAAGCCCCTGCTGAGACTGAA | |
| | | GGGCTTCCCCAGCTTCCCTCTGGTGGAGAGACAGGCA | |
| | | AATGAAGTGGACTGGTGGGACATGGTGTGTAACGTG | |
| | | AAGAAGCTGATCAATGAGAAGAAGGAGGACGGCAAA | |
| | | GTGTTCTGGCAGAATCTGGCCGGCTACAAGCGTCAGG | |
| | | AGGCCCTGCGGCCCTACCTGAGCAGCGAGGAAGACA | |
| | | GAAAGAAGGGCAAGAAGTTCGCCCGGTATCAGCTGG | |
| | | GGGACCTGCTGCTGCACCTCGAGAAGAAGCACGGCG | |
| | | AAGACTGGGGGAAGGTGTACGATGAGGCCTGGGAGC | |
| | | GGATCGATAAGAAGGTGGAGGGCCTGAGCAAGCACA | |
| | | TCAAGCTGGAGGAGGAACGGAGATCTGAGGACGCCC | |
| | | AGAGCAAGGCCGCCCTGACCGACTGGCTGAGAGCCA | |
| | | AGGCCAGCTTCGTCATCGAGGGGCTGAAGGAGGCCG | |
| | | ACAAGGACGAGTTCTGCCGGTGCGAACTGAAGCTGC | |
| | | AGAAGTGGTACGGAGATCTGAGAGGCAAACCTTTCG | |
| | | CCATCGAGGCCGAGAACAGCATCCTGGACATCAGCG | |
| | | GCTTCAGCAAGCAGTACAACTGCGCCTTTATTTGGCA | |
| | | GAAGGACGGAGTGAAGAAGCTGAACCTGTACCTGAT | |
| | | CATCAACTATTTCAAGGGCGGCAAGCTGAGATTCAAG | |
| | | AAGATCAAGCCTGAAGCCTTCGAGGCCAACAGATTCT | |
| | | ACACCGTGATTAACAAGAAAAGCGGAGAGATCGTGC | |
| | | CAATGGAAGTGAACTTCAACTTCGACGACCCTAACCT | |
| | | GATCATCCTGCCCCTGGCATTTGGCAAGCGGCAGGGC | |
| | | AGAGAGTTCATCTGGAACGACCTGCTGTCTCTGGAGA | |
| | | CCGGCAGCCTGAAGCTGGCCAACGGCAGAGTGATCG | |
| | | AGAAGACACTGTACAACAGACGAACCAGACAAGACG | |
| | | AGCCCGCCCTGTTTGTGGCCCTGACCTTCGAGAGAAG | |
| | | AGAGGTGCTGGACAGCAGCAATATCAAGCCTATGAA | |
| | | CCTGATCGGCGTGGACCGGGGCGAGAACATCCCTGC | |
| | | CGTGATCGCCCTTACCGACCCCGAGGGATGCCCTCTG | |
| | | AGCCGGTTTAAAGACAGCCTGGGCAACCCTACCCAC | |
| | | ATCCTGAGAATTGGCGAGTCCTACAAGGAGAAGCAG | |
| | | AGAACCATCCAGGCCAAGAAGGAGGTGGAGCAGCGG | |
| | | CGGGCTGGCGGCTACTCCCGGAAGTACGCCAGCAAG | |
| | | GCCAAGAACCTGGCCGACGACATGGTTAGAAATACC | |
| | | GCCAGAGACCTCCTGTACTACGCTGTGACCCAGGACG | |
| | | CCATGCTGATCTTCGAGAACCTGAGCAGAGGCTTCGG | |
| | | CAGACAGGGCAAGAGAACCTTCATGGCCGAGAGACA | |
| | | GTACACCCGGATGGAGGACTGGCTGACCGCCAAGCT | |
| | | GGCCTACGAGGGCCTGCCCTCTAAGACCTACCTGTCC | |
| | | AAGACCTTGGCACAGTACACCAGCAAGACATGCTCT | |
| | | AACTGCGGCTTCACAATCACGAGCGCCGACTACGACC | |
| | | GGGTGCTGGAGAAACTGAAGAAGACCGCCACAGGCT | |
| | | GGATGACCACCATTAACGGCAAGGAGCTGAAGGTGG | |
| | | AGGGCCAGATCACCTACTACAACAGGTACAAACGGC | |
| | | AGAACGTGGTGAAGGACCTGAGCGTGGAACTGGATA | |
| | | GACTGAGCGAGGAAAGCGTAAACAATGACATCAGCA | |
| | | GCTGGACCAAGGGCCGGAGCGGCGAGGCCCTGAGCC | |
| | | TGCTGAAGAAGAGATTCTCCCACAGACCAGTGCAGG | |
| | | AGAAGTTCGTGTGTCTGAACTGCGGCTTCGAGACCCA | |
| | | CGCCGACGAGCAAGCCGCCCTGAACATCGCCCGGTCT | |
| | | TGGCTTTTCCTGCGGAGCCAGGAGTACAAGAAGTACC | |
| | | AGACAAACAAGACCACAGGCAACACAGACAAGAGA | |
| | | GCCTTCGTCGAGACCTGGCAGAGCTTCTACAGAAAGA | |
| | | AGCTGAAGGAGGTGTGGAAGCCTGCCGTG | |
| c-MYC | | GGAAGCCCCGCTGCCAAGAGAGTGAAGCTGGACTAA | 19753 |
| NLS + | | | |
| STOP codon | | | |
| 3′UTR | Mouse HBA | GCTGCCTTCTGCGGGGCTTGCCTTCTGGCCATGCCCTT | 19740 |
| | | CTTCTCTCCCTTGCACCTGTACCTCTTGGTCTTTGAAT | |
| | | AAAGCCTGAGTAGGAAG | |
| XbaI | | TCTAG | 19741 |
| restriction | | | |
| site (partial) | | | |
| Poly(A) tail | | AAAAAAAAAAAAAAAAAAAAAAAAAAAAAAAAAAA | 19826 |
| (79) | | AAAAAAAAAAAAAAAAAAAAAAAAAAAAAAAAAAA | |
| | | AAAAAAAAA | |

*Components are listed in a 5′ to 3′ order within the constructs

TABLE 46
Full-length RNA sequence of chemically modified CasX 515 mRNA molecule
assessed in this example.*

| CasX | | SEQ ID |
|---|---|---|
| mRNA | RNA sequence | NO |
| CasX 515 | AAAmψAAGAGAGAAAAGAAGAGmψAAGAAGAAAmψAmψAAGAGCCACC | 19755 |
| mRNA | AmψGGCCCCmψGCmψGCCAAGAGAGmψGAAGCmψGGAmψAGCAGACAG | |
| | GAGAmψCAAGCGGAmψmψAAmψAAAAmψmψCGGAGAAGACmψGGmψGA | |
| | AGGAmψmψCmψAACACAAAGAAGGCmψGGCAAGACAGGCCCmψAmψGA | |
| | AGACACmψGCmψGGmψGAGAGmψGAmψGACACCCGACCmψGAGAGAAA | |
| | GACmψGGAAAACCmψGAGAAAGAAGCCmψGAGAAmψAmψCCCCCAGCCC | |
| | AmψCAGCAACACAAGCCGGGCCAACCmψGAAmψAAGCmψGCmψGACCGA | |
| | CmψACACCGAAAmψGAAGAAGGCCAmψCCmψGCACGmψGmψAmψmψGG | |
| | GAAGAGmψmψCCAGAAAGACCCAGmψCGGCCmψGAmψGAGCAGAGmψG | |
| | GCmψCAGCCmψGCCAGCAAGAAGAmψCGAmψCAGAACAAGCmψGAAGCC | |
| | CGAAAmψGGACGAGAAGGGGAACCmψGACAACCGCCGGCmψmψmψGCC | |
| | mψGmψAGCCAGmψGCGGCCAGCCCCmψGmψmψmψGmψGmψACAAACmψG | |
| | GAACAGGmψGAGCGAAAAGGGCAAGGCmψmψACACGAAmψmψACmψmψ | |
| | CGGCAGAmψGCAACGmψGGCCGAGCACGAGAAGCmψGAmψCCmψGCmψ | |
| | GGCCCAGCmψGAAGCCmψGAGAAGGAmψAGCGAmψGAGGCAGmψGACA | |
| | mψAmψmψCCCmψGGGCAAGmψmψCGGACAGCGGGCCCmψGGAmψmψmψ | |
| | mψmψAmψmψCCAmψmψCAmψGmψGACCAAGGAAmψCCACCCACCCCGmψ | |
| | CAAGCCmψCmψmψGCCCAAAmψmψGCCGGCAACAGAmψACGCCAGCGGC | |
| | CCCGmψGGGCAAGGCCCmψGAGCGACGCCmψGmψAmψGGGCACCAmψCG | |
| | CCAGCmψmψCCmψGmψCmAAGmψACCAGGACAmψmψAmψCAmψCGAGC | |
| | ACCAGAAGGmψGGmψGAAGGGCAACCAGAAGAGACmψGGAGAGCCmψG | |
| | CGCGAGCmψGGCCGGCAAGGAAAACCmψGGAGmψAmψCCmψAGCGmψG | |
| | ACCCmψGCCmψCCmψCAGCCmψCAmψACAAAGGAGGGCGmψGGAmψGCC | |
| | mψACAACGAAGmψGAmψCGCCCGGGmψGCGGAmψGmψGGGmψGAACCm | |
| | ψGAAmψCmψGmψGGCAGAAGCmψGAAGCmψGmψCmψAGAGACGACGCC | |
| | AAGCCCCmψGCmψGAGACmψGAAGGGCmψmψCCCCAGCmψmψCCCmψCm | |
| | ψGGmψGGAGAGACAGGCAAAmGAAGmψGGACmψGGmψGGGACAmψGG | |
| | mψGmψGmψAACGmψGAAGAAGCmψGAmψCAAmψGAGAAGAAGGAGGAC | |
| | GGCAAAGmψGmψmψCmψGGCAGAAmψCmψGGCCGGCmψACAAGCGmψC | |
| | AGGAGGCCCmψGCGGCCCmψACCmψGAGCAGCGAGGAAGACAGAAAGA | |
| | AGGGCAAGAAGmψmψCGCCCGGmψAmψCAGCmψGGGGGACCmψGCmG | |
| | CmψGCACCmψCGAGAAGAAGCACGGCGAAGACmψGGGGGAAGGmψGmψ | |
| | ACGAmψGAGGCCmψGGGAGCGGAmψCGAmψAAGAAGGmψGGAGGGCCm | |
| | ψGAGCAAGCACAmψCAAGCmψGGAGGAGGAACGGAGAmψCmψGAGGAC | |
| | GCCCAGAGCAAGGCCGCCCmψGACCGACmψGGCmψGAGAGCCAAGGCCA | |
| | GCmψmψCGmψCAmψCGAGGGGCmψGAAGGAGGCCGACAAGGACGAGmψ | |
| | mψCmψGCCGGmψGCGAACmψGAAGCmψGCAGAAGmψGGmψACGGAGAm | |
| | ψCmψGAGAGGCAAACCmψmψmψCGCCAmψCGAGGCCGAGAACAGCAmψC | |
| | CmψGGACAmψCAGCGGCmψmψCAGCAAGCAGmψACAACmψGCGCCmψmψ | |
| | mψAmψmψmψGGCAGAAGGACGGAGmψGAAGAAGCmψGAACCmψGmψAC | |
| | CmψGAmψCAmψCAACmψAmψmψmψCAAGGGCGGCAAGCmψGAGAmψmψ | |
| | CAAGAAGAmψCAAGCCmψGAAGCCmψmψCGAGGCCAACAGAmψmψCmψ | |
| | ACACCGmψGAmψmψAACAAGAAAAGCGGAGAGAmψCGmψGCCAAmψGG | |
| | AAGmψGAACmψmψCAACmψmψCGACGACCCmψAACCmψGAmψCAmψCCm | |
| | ψGCCCCmψGGCAmψmψmψGGCAAGCGGCAGGGCAGAGAGmψmψCAmψCm | |
| | ψGGAACGACCmψGCmψGmψCmψCmψGGAGACCGGCAGCCmψGAAGCmψG | |
| | GCCAACGGCAGAGmψGAmψCGAGAAGACACmψGmψACAACAGACGAAC | |
| | CAGACAAGACGAGCCCGCCCmGmψmψmψGmψGGCCCmψGACCmψmψCG | |
| | AGAGAAGAGAGGmψGCmψGGACAGCAGCAAmψAmψCAAGCCmψAmψGA | |
| | ACCmψGAmψCGGCGmψGGACCGGGGCGAGAACAmψCCCmψGCCGmψGA | |
| | mψCGCCCmψmψACCGACCCCGAGGGAmψGCCCmψCmψGAGCCGGmψmψm | |
| | ψAAAGACAGCCmψGGGCAACCCmψACCCACAmψCCmψGAGAAmψmψGGC | |
| | GAGmψCCmψACAAGGAGAAGCAGAGAACCAmψCCAGGCCAAGAAGGAG | |
| | GmψGGAGCAGCGGCGGGCmψGGCGGCmψACmψCCCGGAAGmψACGCCAG | |
| | CAAGGCCAAGAACCmGGCCGACGACAmGGmψmψAGAAAmψACCGCCA | |
| | GAGACCmψCCmψGmψACmψACGCmψGmψGACCCAGGACGCCAmψGCmψG | |
| | AmψCmψmψCGAGAACCmψGAGCAGAGGCmψmψCGGCAGACAGGGCAAG | |
| | AGAACCmψmψCAmψGGCCGAGAGACAGmψACACCCGGAmψGGAGGACm | |
| | ψmψGGCmψGACCGCCAAGCmψGGCCmψACGAGGGCCmψGCCCmψCmψAAGA | |
| | CCmψACCmψGmψCCAAGACCmψmψGGCACAGmψACACCAGCAAGACAmψ | |
| | GCmψCmAACmψGCGGCmψmψCACAAmψCACGAGCGCCGACmψACGACC | |
| | GGGmψGCmψGGAGAAACmψGAAGAAGACCGCCACAGGCmψGGAmψGAC | |
| | CACCAmψmψAACGGCAAGGAGCmψGAAGGmψGGAGGGCCAGAmψCACC | |
| | mψACmψACAACAGGmψACAAACGGCAGAACGmψGGmψGAAGGACCmψG | |
| | AGCGmψGGAACmψGGAmψAGACmψGAGCGAGGAAAGCGmψAAACAAmψ | |
| | GACAmψCAGCAGCmψGGACCAAGGGCCGGAGCGGCGAGGCCCmψGAGCC | |
| | mψGCmψGAAGAAGAGAmψmψCmmψCCCACAGACCAGmψGCAGGAGAAGm | |
| | ψmψCGmψGmψGmψCmψGAACmψGCGGCmψmψCGAGACCCACGCCGACGA | |
| | GCAAGCCGCCCmψGAACAmψCGCCCGGmψCmψmψGGCmψmψmψmψCCmψ | |
| | GCGGAGCCAGGAGmψACAAGAAGmψACCAGACAAACAAGACCACAGGC | |
| | AACACAGACAAGAGAGCCmψmψCGmψCGAGACCmψGGCAGAGCmψmψC | |
| | mψACAGAAAGAAGCmψGAAGGAGGmψGmψGGAAGCCmψGCCGmψGGGA | |
| | AGCCCCGCmψGCCAAGAGAGmψGAAGCmψGGACmψAAGCmψGCCmψmψC | |
| | mψGCGGGGCmψmGCCmψmψCmψGGCCAmψGCCCmψmψCmψmψCmψCmψ | |
| | CCCmψmψGCACCmψGmψACCmψCmψmψGGmψCmψmψmψGAAmψAAAGCC | |
| | mψGAGmψAGGAAGmψCmψAGAAAAAAAAAAAAAAAAAAAAAAAAAAAA | |
| | AAAAAAAAAAAAAAAAAAAAAAAAAAAAAAAAAAAAAAAAAAAAAAA | |
| | AAAAA | |

The CleanCap ® AG 5′ cap is not shown in the table. Modification ‘mψ’ = N1-methyl-pseudouridine

TABLE 47
Full-length protein sequence of CasX 515 + NLS molecule.

| | | SEQ ID |
|---|---|---|
| CasX | AA sequence | NO |
| CasX 515 | MAPAAKRVKLDSRQEIKRINKIRRRLVKDSNTKKAGKTGPMKTLLVRVMTP | 19757 |
| with | DLRERLENLRKKPENIPQPISNTSRANLNKLLTDYTEMKKAILHVYWEEFQK | |
| flanking | DPVGLMSRVAQPASKKIDQNKLKPEMDEKGNLTTAGFACSQCGQPLFVYK | |
| c-MYC | LEQVSEKGKAYTNYFGRCNVAEHEKLILLAQLKPEKDSDEAVTYSLGKFGQ | |
| NLSes | RALDFYSIHVTKESTHPVKPLAQIAGNRYASGPVGKALSDACMGTIASFLSK | |
| | YQDIIIEHQKVVKGNQKRLESLRELAGKENLEYPSVTLPPQPHTKEGVDAYN | |
| | EVIARVRMWVNLNLWQKLKLSRDDAKPLLRLKGFPSFPLVERQANEVDW | |
| | WDMVCNVKKLINEKKEDGKVFWQNLAGYKRQEALRPYLSSEEDRKKGKK | |
| | FARYQLGDLLLHLEKKHGEDWGKVYDEAWERIDKKVEGLSKHIKLEEERR | |
| | SEDAQSKAALTDWLRAKASFVIEGLKEADKDEFCRCELKLQKWYGDLRGK | |
| | PFAIEAENSILDISGFSKQYNCAFIWQKDGVKKLNLYLIINYFKGGKLRFKKI | |
| | KPEAFEANRFYTVINKKSGEIVPMEVNFNFDDPNLIILPLAFGKRQGREFIWN | |
| | DLLSLETGSLKLANGRVIEKTLYNRRTRQDEPALFVALTFERREVLDSSNIKP | |
| | MNLIGVDRGENIPAVIALTDPEGCPLSRFKDSLGNPTHILRIGESYKEKQRTIQ | |
| | AKKEVEQRRAGGYSRKYASKAKNLADDMVRNTARDLLYYAVTQDAMLIF | |
| | ENLSRGFGRQGKRTFMAERQYTRMEDWLTAKLAYEGLPSKTYLSKTLAQY | |
| | TSKTCSNCGFTITSADYDRVLEKLKKTATGWMTTINGKELKVEGQITYYNR | |
| | YKRQNVVKDLSVELDRLSEESVNNDISSWTKGRSGEALSLLKKRFSHRPVQ | |
| | EKFVCLNCGFETHADEQAALNIARSWLFLRSQEYKKYQTNKTTGNTDKRAF | |
| | VETWQSFYRKKLKEVWKPAVGSPAAKRVKLD | |

TABLE 48
gRNAs with LPA-targeting targeting sequences used in this example*

| | | SEQ ID |
|---|---|---|
| gRNA ID | RNA Sequence | NO. |
| 316-TG-48- | mA*mC*mU*GGCGCUUCUAUCUGAUUACUCUGAGCGCCAUCACCAGCG | 19855 |
| 351 (v1) | ACUAUGUCGUAGUGGGUAAAGCUCCCUCUUCGGAGGGAGCAUCAGAGC | |
| | AAUAACAAGGAGCUGG*mG*mC*mU | |
| 316-TG-48- | mA*mC*mU*GGCGCUUCUAUCUGAUUACUCUGAGCGCCAUCACCAGCG | 19856 |
| 222 (v1) | ACUAUGUCGUAGUGGGUAAAGCUCCCUCUUCGGAGGGAGCAUCAGAGU | |
| | GUGAUAGUGGUGGAGA*mG*mU*mG | |
| 316-TG-48- | mA*mC*mU*GGCGCUUCUAUCUGAUUACUCUGAGCGCCAUCACCAGCG | 19857 |
| 317 (v1) | ACUAUGUCGUAGUGGGUAAAGCUCCCUCUUCGGAGGGAGCAUCAGAGU | |
| | GGAGCCCACACAAGCA*mG*mA*mU | |
| 316-TG-48- | mA*mC*mU*GGCGCUUCUAUCUGAUUACUCUGAGCGCCAUCACCAGCG | 19858 |
| 284 (v1) | ACUAUGUCGUAGUGGGUAAAGCUCCCUCUUCGGAGGGAGCAUCAGAGA | |
| | GGGACAAAUAAAUGGG*mC*mA*mG | |
| 316-TG-48- | mA*mC*mU*GGCGCUUCUAUCUGAUUACUCUGAGCGCCAUCACCAGCG | 19859 |
| 107 (v1) | ACUAUGUCGUAGUGGGUAAAGCUCCCUCUUCGGAGGGAGCAUCAGAGU | |
| | CUGCCUUUCUACUGGA*mU*mA*mC | |
| 316-TG-48- | mA*mC*mU*GGCGCUUCUAUCUGAUUACUCUGAGCGCCAUCACCAGCG | 19860 |
| 178 (v1) | ACUAUGUCGUAGUGGGUAAAGCUCCCUCUUCGGAGGGAGCAUCAGAGU | |
| | CAGAAGAAGCCUCUGU*mG*mC*mU | |
| 316-TG-48- | mA*mC*mU*GGCGCUUCUAUCUGAUUACUCUGAGCGCCAUCACCAGCG | 19861 |
| 303 (v1) | ACUAUGUCGUAGUGGGUAAAGCUCCCUCUUCGGAGGGAGCAUCAGAGU | |
| | UCUAGGUUUGGAAAGC*mA*mC*mU | |
| 316-TG-48- | mA*mC*mU*GGCGCUUCUAUCUGAUUACUCUGAGCGCCAUCACCAGCG | 19862 |
| 305 (v1) | ACUAUGUCGUAGUGGGUAAAGCUCCCUCUUCGGAGGGAGCAUCAGAGA | |
| | GCAGUGAGCAGCAGUC*mA*mG*mC | |

*each targeting sequence was modified at the 3′ terminal end with phosphorothioate bonds and 2′OMe modification of the 3 terminal nucleotides

TABLE 49
Apo(a) levels relative to non-targeting
gRNA in primary human hepatocytes

| Targeting | Emax | Emax Protein | EC₅₀ Protein | EC₅₀ |
|---|---|---|---|---|
| sequence ID | Editing | (Jess) | (Jess) | Editing |
| TG-48-351 | 90.96 | 87.75 | 0.072 | 0.053 |
| TG-48-317 | 80.86 | 73.36 | 0.065 | 0.062 |
| TG-48-284 | 80.54 | 82.38 | 0.106 | 0.109 |
| TG-48-222 | 79.29 | 81.31 | 0.143 | 0.122 |
| TG-48-239 | 67.21 | 95.4 | 0.045 | 0.117 |
| TG-48-303 | 77.82 | 82.34 | 3.510 | 1.976 |
| TG-48-178 | 55.98 | 43.66 | 0.081 | 0.161 |
| TG-48-107 | 89.51 | 55.76 | 0.062 | 0.024 |

TABLE 50
Top 4 LPA-targeting sequences

| Targeting | | SEQ |
|---|---|---|
| sequence ID | Targeting sequence (RNA) | ID NO: |
| TG-48-351 | CAAUAACAAGGAGCUGGGCU | 901 |
| TG-48-222 | UGUGAUAGUGGUGGAGAGUG | 817 |
| TG-48-317 | UGGAGCCCACACAAGCAGAU | 898 |
| TG-48-284 | AGGGACAAAUAAAUGGGCAG | 826 |

### Example 13: Evaluation of LPA Targeting in a Mouse Model

Experiments are performed to assess editing rates and protein production upon treatment with a CasX:gRNA system when delivered in vivo in a mouse model. Four LPA-targeting spacers are evaluated for editing rate at the LPA locus, LPA transcript knockdown, and apo (a) protein production using the CasX 515 protein.

### Materials and Methods

### Lnp Preparation:

CasX 515 mRNA and gRNA is encapsulated into LNPs using ALC-0315 based lipid mix. After formulation, the LNPs are dialyzed into PBS, pH 7.4, to decrease the ethanol concentration and increase the pH, which increases the stability of the particles. Buffer exchange of the mRNA/gRNA-LNPs is achieved by overnight dialysis into PBS, pH 7.4, at 4° C. using 10k Slide-A-Lyzer™ Dialysis or Cassettes (Thermo Scientific™) or 12-14 kDa dialysis tubing (Repligen). Formulated LNPs are analyzed on a Malvern Zetasizer to determine their diameter and polydispersity index (PDI). Encapsulation efficiency and RNA concentration is determined by RiboGreen™ assay using Invitrogen's Quant-iT™ RiboGreen™ RNA assay kit. The LNPs described above are used to deliver CasX 515 mRNA and gRNA for delivery to target tissues in vivo.

Delivery of LNPs Encapsulating CasX mRNA and Targeting gRNA In Vivo:

To assess the effects of LPA targeting in vivo, LPA-targeting gRNA using scaffold 316 with spacers from Table 50 are encapsulated within the same LNP using a 1:1 mass ratio for mRNA:gRNA. Formulated LNPs are buffer-exchanged to PBS for in vivo injection. Briefly, LNPs are administered intravenously through the tail-vein into 20-week-old Pxb mice at 3 mg/Kg. Naïve, uninjected animals serve as negative experimental controls. Fourteen days post-administration, mice are euthanized, and the blood and liver tissue are harvested. Blood serum is collected for apo (a) Western blot via Jess, liver tissue is homogenized for apo (a) protein Western blot, mRNA extraction and for gDNA extraction using the Zymo Research Quick DNA/RNA Miniprep kit following the manufacturer's instructions.

### NGS Processing and Analysis:

Extracted gDNA is processed and analyzed as described in Example 12. qPCR assay for LPA mRNA knockdown:

Extracted mRNA is processed and analyzed RT-qPCR. Measurement of LPA mRNA expression is performed using a single-step RT-qPCR approach. 25-50 uL of sample lysate is used for RNA extraction. GAPDH primers/probe are used for normalizing transcript levels (IDT; Hs.PT.39a.22214836). Percent change in mRNA is calculated by comparing to samples treated with a non-targeting sequence.

### Western Blot by Automated Capillary Separation and Detection System, Jess (Bio-Techne):

Serum and liver tissue from animal samples is assayed as described in Example 12.

The experiments are expected to provide results quantifying editing, mRNA knockdown, and therapeutic levels of apo (a) reduction in a mouse model.

1. A system comprising a chimeric engineered CasX protein and a guide ribonucleic acid (gRNA), wherein the gRNA comprises a scaffold sequence comprising a sequence selected from the group consisting of SEQ ID NOS: 464-466, or a sequence having at least about 90% sequence identity thereto, and a targeting sequence complementary to a lipoprotein (a) (LPA) gene target nucleic acid sequence.

2-5. (canceled)

6. The system of claim 1, wherein the targeting sequence of the gRNA comprises a sequence selected from the group consisting of the sequences of SEQ ID NOS: 762-903 or a sequence having at least about 80% identity thereto.

7-33. (canceled)

34. The system of claim 1, wherein the gRNA is chemically modified.

35. The system of claim 34, wherein the chemical modification to the gRNA is an addition of a 2′O-methyl group to one or more nucleotides of the gRNA.

36. The system of claim 34, wherein the chemical modification to the gRNA is a substitution of a phosphorothioate bond between two or more nucleotides of the gRNA.

37. The system of claim 1, wherein the chemical modification is an addition of a 2′O-methyl group to the 3 nucleotides of the 5′ and 3′ ends of the gRNA and wherein each of the modified nucleotides is linked to the adjoining nucleotide by a phosphorothioate bond.

38. The system of claim 34, wherein the chemically modified gRNA comprises a sequence selected from the group consisting of SEQ ID NOS: 19855-19862, or a sequence having at least about 80% sequence identity thereto.

39. (canceled)

40. The system of claim 34, wherein the chemically modified gRNA comprises a sequence of SEQ ID NO: 19855.

41. The system of claim 1, wherein the gRNA is capable of forming a ribonucleoprotein (RNP) with the chimeric engineered CasX protein and binding the target nucleic acid.

42. (canceled)

43. The system of claim 1, wherein the chimeric engineered CasX protein comprises a sequence selected from the group consisting of SEQ ID NOS: 4-7, or a sequence having at least about 90% sequence identity thereto.

44-47. (canceled)

48. The system of claim 1, wherein the chimeric engineered CasX protein comprises one or more nuclear localization signals (NLS).

49. The system of claim 48, wherein the NLS is a simian virus 40 (SV40) NLS or a c-MYC NLS.

50. A nucleic acid comprising a sequence that encodes the gRNA of claim 1.

51-68. (canceled)

69. A lipid nanoparticle (LNP) comprising:
a. a chimeric engineered CasX variant protein or a sequence encoding the chimeric engineered CasX variant protein; and
b. a gRNA comprising a targeting sequence complementary to an LPA gene target nucleic acid sequence or a sequence encoding the gRNA.

70. A lipid nanoparticle (LNP) comprising the nucleic acid of claim 50.

71. (canceled)

72. The LNP of claim 69, wherein the LNP comprises a component selected from the group consisting of one or more ionizable lipids, one or more helper phospholipids, one or more polyethylene glycol (PEG)-modified lipids, and/or cholesterol or a derivative thereof.

73. A vector comprising the nucleic acid of claim 50, wherein the vector is selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, a DNA vector, and an RNA vector.

74-76. (canceled)

77. A pharmaceutical composition comprising
the system of claim 1.

78. (canceled)

79. A method of modifying an LPA target nucleic acid sequence in a population of cells, the method comprising introducing into cells of the population:
a. system comprising a chimeric engineered CasX protein and a guide ribonucleic acid (gRNA); or
b. a lipid nanoparticle (LNP) comprising a chimeric engineered CasX variant protein or a sequence encoding the chimeric engineered CasX variant protein; or a sequence encoding the gRNA;
wherein the gRNA comprises a scaffold sequence comprising a sequence selected from the group consisting of SEQ ID NOS: 464-466, or a sequence having at least about 90% sequence identity thereto, and a targeting sequence complementary to a lipoprotein (a) (LPA) gene target nucleic acid sequence.

80-96. (canceled)

97. A method of treating an LPA-related disease in a subject in need thereof, comprising administering to the subject a therapeutically effective dose of:
a. a system comprising a chimeric engineered CasX protein and a guide ribonucleic acid (gRNA); or
b. a lipid nanoparticle (LNP) comprising a chimeric engineered CasX variant protein or a sequence encoding the chimeric engineered CasX variant protein; or a sequence encoding the gRNA;
wherein the gRNA comprises a scaffold sequence comprising a sequence selected from the group consisting of SEQ ID NOS: 464-466, or a sequence having at least about 90% sequence identity thereto, and a targeting sequence complementary to a lipoprotein (a) (LPA) gene target nucleic acid sequence.

98-121. (canceled)
